(19) United States
(12) Patent Application Publication
(10) Pub. No.: US 2022/0347173 A1
(43) Pub. Date: Nov. 3, 2022

(54) METHODS OF TREATING LIVER FIBROSIS USING CALPAIN INHIBITORS

(71) Applicant: Blade Therapeutics, Inc., South San Francisco, CA (US)

(72) Inventors: Sharlene Lim, San Mateo, CA (US); Prabha Ibrahim, Mountain View, CA (US); Maria Fuentes, San Francisco, CA (US)

(21) Appl. No.: 17/255,168

(22) PCT Filed: Jun. 27, 2019
(86) PCT No.: PCT/US2019/039597

§ 371 Date: Dec. 22, 2020

Related U.S. Application Data
(60) Provisional application No. 62/691,510, filed on Jun. 28, 2018.

(51) Int. Cl. A61K 31/498; A61K 31/421; A61K 31/433; A61K 31/4439; A61K 31/166; A61K 31/415; A61P 1/16; A61K 45/06

(52) U.S. Cl. CPC A61K 31/498; A61K 31/421; A61K 31/433; A61K 45/06; A61K 31/166; A61K 31/415; A61P 1/16; A61K 31/4439

(57) ABSTRACT

Disclosed herein are methods of treating liver fibrosis by administering calpain inhibitors to subjects in need thereof.

### BACKGROUND

### Field

The present application relates to the fields of pharmaceutical chemistry, biochemistry, and medicine. More particularly, the present invention relates to calpain inhibitors and their use as therapeutic agents.

### Description

Fibroproliferative disorders are main contributors to organ impairment resulting in substantial morbidity and mortality. Liver fibrosis is a result of acute or chronic liver injury. It could be the response to metabolic, viral, or toxic stimuli, among others. Calpain inhibition can potentially be beneficial in multiple hepatic fibrotic disease.

Primary sclerosing cholangitis (PSC) is a rare, chronic, progressive disease characterized by inflammation and subsequent destruction of intra- and extrahepatic bile ducts. Over time, patients develop liver fibrosis and cirrhosis, which ultimately can lead to liver failure. PSC is also associated with increased rates of colorectal, hepatobiliary, and gallbladder cancer (Kumar et al., 2016, Clin Med Insights Gastroenterol. 9:25-29). Epidemiology studies indicate that there may be up to 50,000 individuals afflicted with PSC in the US (Ali et al., 2015. Intractable Rare Dis Res. 4(1):1-6), although prevalence rates are generally presumed to be underestimated due to the difficulty of correctly diagnosing asymptomatic patients (Eksteen, 2014. Br Med Bull. 110(1):89-98). Disease management primarily entails symptomatic treatment (for example, of pruritus and fatigue), but there are no FDA-approved agents to treat PSC, and no therapies have been shown to consistently slow disease progression. An anti-fibrotic agent that effectively delays disease progression would be of tremendous benefit to individuals with PSC.

Primary biliary cholangitis (PBC; formerly known as primary biliary cirrhosis) is an autoimmune disease characterized by the gradual destruction of interlobular bile ducts. While etiology is unknown, bile duct degradation leads to accumulation of toxic bile acids, eventually leading to fibrosis, cirrhosis, and ultimately liver failure. PBC disproportionately afflicts females (at a gender ratio of ˜10:1), with diagnosis typically made between 40-60 years of age when patients are asymptomatic (Selmi et al. 2004. J Clin Gastroenterol. 38:264-271). Disease progression is highly variable and difficult to predict, although untreated early stage disease may progress to cirrhosis within 4-6 years (Washington 2007, Modern Pathol. 20:S15-S30). Epidemiology varies by geography, and a frequently cited Mayo study estimates a US prevalence rate of 40 PBC patients/100 K, translating to a US prevalence of 120-130 K (Kim et al. 2000. Gastroenterol. 119:1631-1636). Ursodeoxycholic acid (UDCA) is generic and is the standard first-line treatment for PBC, resulting in disease stabilization for ˜50% of patients. Obeticholic acid (OCA), a bile acid FXR agonist developed by Intercept Pharmaceuticals, was approved in the US in 2016 in patients with inadequate response or intolerant to UDCA. While OCA is efficacious at improving liver histology in many of these patients, use is associated with increased LDL levels and pruritus (Neuschwander-Tetri et al. 2015, Lancet. 385:956-65), leaving opportunity for future products in development.

Liver cirrhosis is a late stage of hepatic fibrosis characterized by diffuse nodular regeneration, collapse of liver structures, and substantial hepatic vasculature architectural distortion. This loss of functional architecture leads directly to increased portal hypertension, which itself is the primary driver of complications including ascites, hepatic encephalopathy, and variceal formation (Tsochatzis et al. 2014, Lancet. 383:1749-1761; Goldberg and Chopra, 2017, UpToDate Cirrhosis in adults: Overview of complications, general management, and prognosis). Once patients develop major complications, they are considered decompensated, after which the only treatment for many patients is liver transplant. In addition to NASH, PBC, and PSC, there are a large number of causes of cirrhosis, including alcoholic liver disease, alpha-1 antitrypsin deficiency, autoimmune hepatitis, celiac disease, chronic viral hepatitis, hemochromatosis, idiopathic portal fibrosis, and Wilson disease (Goldberg and Chopra, 2016, UpToDate Cirrhosis in adults: Etiologies, clinical manifestations, and diagnosis). There are currently a lack of effective treatments for cirrhotic patients, but opportunity exists to both delay progression and to reverse liver degeneration/fibrosis. This could be approached in both patients in which the driver of liver disease has been removed and in which the driver remains active. The first group includes viral patients (HCV, HBV) that have exhibited a sustained virologic response or alcoholic hepatitis patients that remain abstinent. These patients would theoretically be best-positioned to exhibit improved liver fibrosis after a treatment period due to the absence of an ongoing insult. Patients with etiologies continuing to actively drive liver degeneration stand to benefit from a therapy that delays disease progression, development of complications, transition to decompensation, and end stage liver failure.

It is estimated that up to one-third of the populations in the US and Europe have a condition termed non-alcoholic fatty liver disease (NAFLD), which is characterized by steatosis, or excessive accumulation of fat in the liver (Wree et al. 2013. Nat. Rev. Gastroenterol. Hepatol. 10: 627-636; Blachier et al. 2012. J. Hepatol. 58: 593-608). Many of these individuals, for reasons not totally understood, subsequently develop liver inflammation, or steatohepatitis. This condition, called non-alcoholic steatohepatitis, or NASH, develops in roughly 10-20% of NAFLD patients, accounting for approximately 10-20 million individuals in the US (Schattenburg et al. 2011. Curr Opin Lipidol. 22:479-488). Individuals experiencing chronic liver inflammation often develop liver fibrosis, with eventual risks of cirrhosis, hepatocellular carcinoma, and liver failure. Based on current projections, NASH is predicted to become the leading cause of liver transplantation by 2020. (Wree, 2013). Unfortunately, there are no therapies available to prevent or treat liver fibrosis.

### SUMMARY

Disclosed herein is a method of treating a disease or disorder selected from the group consisting of primary sclerosing cholangitis, primary biliary cholangitis, non-alcoholic fatty liver disease, non-alcoholic steatohepatitis, and liver cirrhosis; the method comprising administering one or more calpain inhibitors, either as a single agent or in combination with other agents, to a subject in need thereof. Examples of agents for combination include, but are not limited to, a VAP-1 inhibitor, an ASBT inhibitor, a dual CCR2/5 antagonist, an anti-cholestatic bile acid, a FXR agonist, a FGFR1c/4 agonist, a CCL24 inhibitor, obeticholic acid, elafibranor, cenicriviroc, selonsertib, a niacin receptor agonist, a SGLT2 inhibitor, and a FGF21 mimetic.

In some embodiments, the liver cirrhosis may be caused by one or more of the conditions selected from the group consisting of alcoholic liver disease, alpha-1 antitrypsin deficiency, autoimmune hepatitis, celiac disease, chronic viral hepatitis, hemochromatosis, idiopathic portal fibrosis, and Wilson disease.

In some embodiments, the calpain inhibitor may be a compound as described herein. In some embodiments, the calpain inhibitor may be a compound of any one of Formula I, II, III, IV, V, VI, VII, VIII, or IX. In some embodiments, the calpain inhibitor is a compound listed in Table 1a, 1b, 2, 3, or 4.

### BRIEF DESCRIPTION OF THE DRAWINGS

FIG. 1 shows mouse liver sections stained with Picrosirius Red (PSR) and viewed using polarized light microscopy.

FIG. 2 summarizes the Fibrosis scores from Picrosirius Red-stained liver sections.

FIG. 3 shows the immunohistochemical evaluation of CAPN1 in normal human liver and in diseased human liver (NASH, cirrhosis, fatty liver disease).

FIG. 4 shows the immunohistochemical evaluation of CAPN1 in normal human liver and in diseased human liver (PSC and PBC).

FIG. 5 shows the immunohistochemical evalatuation of CAP2 in normal human liver and in diseased human liver (NASH, cirrhosis, fatty liver disease).

FIG. 6 shows the immunohistochemical evaluation of CAPN2 in normal human liver and in diseased human liver (PSC and PBC).

FIG. 7 shows the immunohistochemical evaluation of CAPN9 in normal human liver and in diseased human liver (NASH, cirrhosis, fatty liver disease).

FIG. 8 shows the immunohistochemical evaluation of CAPN9 in normal human liver and in diseased human liver (PSC and PBC).

FIG. 9A shows Hematoxylin and eosin (H&E)-stained and Sirius Red-stained liver fed a choline-deficient, amino acid-defined high fat diet (CDAHFD), FIGS. 9B-9E shows the expression of smooth musle actin (SMA), collagen (Col1a1), Calpain1, and Calpain 2, respectively, in CDAHFD rats.

FIGS. 10A-10C show the effects of administering Compound 405 once daily in CDAFHD rats on body weight, liver/body weight ration and spleen to body weight ratio, respectively.

FIGS. 11A-1E show the effects of administering Compound 405 once daily in CDAFHD rats on alanaine transfersase (ALT) levels (FIG. 11A), alkaline phosphatase (ALP) levels (FIG. 11B), aspartate transaminase (AST) levels (FIG. 11C), total bilirubin levels (FIG. 11D), and total Albumin levels (FIG. 11E).

FIGS. 12A-12C show the effects of administering Compound 405 twice daily in CDAFHD rats on body weight, liver/body weight ration and spleen to body weight ratio, respectively.

FIGS. 13A-13E show the effects of administering Compound 405 once daily in CDAFHD rats on ALT levels (FIG. 13A), ALP levels (FIG. 13B), AST levels (FIG. 13C), total bilirubin levels (FIG. 13D), and total Albumin levels (FIG. 13E).

FIG. 14A shows H&E-stained, Sirius Red-stained and alpha smooth muscle actin (α-SMA)-stained liver from CDAHFD rats treated with Compound 405 once daily at 200 mg/kg and 60 mg/kg. FIGS. 14B-14E show collagen proportional area (CPA %), hydroxyproline levels, α-SMA levels and percent steatosis, respectively, in CDAHFD rats treated with Compound 405 once daily at 200 mg/kg and 60 mg/kg.

FIG. 15A shows H&E-stained, Sirius Red-stained and α-SMA-stained liver from CDAHFD rats treated with Compound 405 twice daily at 100 mg/kg and 30 mg/kg. FIGS. 15B-15E show collagen CPA %, hydroxyproline levels, α-SMA levels and percent steatosis, respectively, in CDAHFD rats with Compound 405 twice daily at 100 mg/kg and 30 mg/kg.

FIGS. 16A-16F show the levels of profibrotic gene expression in CDAHFD rats treated with Compound 405 once daily at 200 mg/kg and 60 mg/kg.

FIGS. 17A-17F show the levels of profibrotic gene expression in CDAHFD rats treated with Compound 405 twice daily at 100 mg/kg and 30 mg/kg.

### DETAILED DESCRIPTION

### Definitions

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

“Subject” as used herein, means a human or a non-human mammal, e.g., a dog, a cat, a mouse, a rat, a cow, a sheep, a pig, a goat, a non-human primate or a bird, e.g., a chicken, as well as any other vertebrate or invertebrate.

The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rats and mice but also includes many other species.

An “effective amount” or a “therapeutically effective amount” as used herein refers to an amount of a therapeutic agent that is effective to relieve, to some extent, or to reduce the likelihood of onset of, one or more of the symptoms of a disease or condition, and includes curing a disease or condition. “Curing” means that the symptoms of a disease or condition are eliminated; however, certain long-term or permanent effects may exist even after a cure is obtained (such as extensive tissue damage).

“Treat,” “treatment,” or “treating,” as used herein refers to administering a pharmaceutical composition for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from a disease or condition, and may include inhibiting the disease or disorder or arresting its development, or ameliorating or alleviating the cause of the disease or disorder.

As used herein, the term “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. Conventional procedures for the selection and preparation of suitable prodrug derivatives are described, for example, in Design of Prodrugs, (ed. H. Bundgaard, Elsevier, 1985), which is hereby incorporated herein by reference in its entirety.

As used herein, the term “pro-drug ester” refers to derivatives of the compounds disclosed herein formed by the addition of any of several ester-forming groups that are hydrolyzed under physiological conditions. Examples of pro-drug ester groups include pivoyloxymethyl, acetoxymethyl, phthalidyl, indanyl and methoxymethyl, as well as other such groups known in the art, including a (5-R-2-oxo-1,3-dioxolen-4-yl)methyl group. Other examples of pro-drug ester groups can be found in, for example, T. Higuchi and V. Stella, in “Pro-drugs as Novel Delivery Systems”, Vol. 14, A.C.S. Symposium Series, American Chemical Society (1975); and “Bioreversible Carriers in Drug Design: Theory and Application”, edited by E. B. Roche, Pergamon Press: New York, 14-21 (1987) (providing examples of esters useful as prodrugs for compounds containing carboxyl groups). Each of the above-mentioned references is herein incorporated by reference in their entirety.

“Metabolites” of the compounds disclosed herein include active species that are produced upon introduction of the compounds into the biological milieu.

“Solvate” refers to the compound formed by the interaction of a solvent and a compound described herein, a metabolite, or salt thereof. Suitable solvates are pharmaceutically acceptable solvates including hydrates.

The term “pharmaceutically acceptable salt” refers to salts that retain the biological effectiveness and properties of a compound, which are not biologically or otherwise undesirable for use in a pharmaceutical. In many cases, the compounds herein are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in WO 87/05297, Johnston et al., published Sep. 11, 1987 (incorporated by reference herein in its entirety).

As used herein, “Cₐ to C_{b}” or “C_{a-b}” in which “a” and “b” are integers refer to the number of carbon atoms in the specified group. That is, the group can contain from “a” to “b”, inclusive, carbon atoms. Thus, for example, a “C₁ to C₄ alkyl” or “C₁₋₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—.

The term “halogen” or “halo,” as used herein, means any one of the radio-stable atoms of column 7 of the Periodic Table of the Elements, e.g., fluorine, chlorine, bromine, or iodine, with fluorine and chlorine being preferred.

As used herein, “alkyl” refers to a straight or branched hydrocarbon chain that is fully saturated (i.e., contains no double or triple bonds). The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 4 carbon atoms. The alkyl group of the compounds may be designated as “C_{1_4} alkyl” or similar designations. By way of example only, “C₁₋₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

As used herein, “haloalkyl” refers to a straight- or branched-chain alkyl group having from 1 to 12 carbon atoms in the chain, substituting one or more hydrogens with halogens. Examples of haloalkyl groups include, but are not limited to, —CF₃, —CHF₂, —CH₂F, —CH₂CF₃, —CH₂CHF₂, —CH₂CH₂F, —CH₂CH₂Cl, —CH₂CF₂CF₃ and other groups that in light of the ordinary skill in the art and the teachings provided herein, would be considered equivalent to any one of the foregoing examples.

As used herein, “alkoxy” refers to the formula —OR wherein R is an alkyl as is defined above, such as “C₁₋₉ alkoxy”, including but not limited to methoxy, ethoxy, n-propoxy, 1-methylethoxy (isopropoxy), n-butoxy, iso-butoxy, sec-butoxy, and tert-butoxy, and the like.

As used herein, “polyethylene glycol” refers to the formula

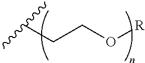

wherein n is an integer greater than one and R is a hydrogen or alkyl. The number of repeat units “n” may be indicated by referring to a number of members. Thus, for example, “2- to 5-membered polyethylene glycol” refers to n being an integer selected from two to five. In some embodiments, R is selected from methoxy, ethoxy, n-propoxy, 1-methylethoxy (isopropoxy), n-butoxy, iso-butoxy, sec-butoxy, and tert-butoxy.

As used herein, “heteroalkyl” refers to a straight or branched hydrocarbon chain containing one or more heteroatoms, that is, an element other than carbon, including but not limited to, nitrogen, oxygen and sulfur, in the chain backbone. The heteroalkyl group may have 1 to 20 carbon atoms although the present definition also covers the occurrence of the term “heteroalkyl” where no numerical range is designated. The heteroalkyl group may also be a medium size heteroalkyl having 1 to 9 carbon atoms. The heteroalkyl group could also be a lower heteroalkyl having 1 to 4 carbon atoms. In various embodiments, the heteroalkyl may have from 1 to 4 heteroatoms, from 1 to 3 heteroatoms, 1 or 2 heteroatoms, or 1 heteroatom. The heteroalkyl group of the compounds may be designated as “C₁₋₄ heteroalkyl” or similar designations. The heteroalkyl group may contain one or more heteroatoms. By way of example only, “C₁₋₄ heteroalkyl” indicates that there are one to four carbon atoms in the heteroalkyl chain and additionally one or more heteroatoms in the backbone of the chain.

The term “aromatic” refers to a ring or ring system having a conjugated pi electron system and includes both carbocyclic aromatic (e.g., phenyl) and heterocyclic aromatic groups (e.g., pyridine). The term includes monocyclic or fused-ring polycyclic (i.e., rings which share adjacent pairs of atoms) groups provided that the entire ring system is aromatic.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms, although the present definition also covers the occurrence of the term “aryl” where no numerical range is designated. In some embodiments, the aryl group has 6 to 10 carbon atoms. The aryl group may be designated as “C₆₋₁₀ aryl,” “C₆ or C₁₀ aryl,” or similar designations. Examples of aryl groups include, but are not limited to, phenyl, naphthyl, azulenyl, and anthracenyl.

As used herein, “aryloxy” and “arylthio” refers to RO— and RS—, in which R is an aryl as is defined above, such as “C₆₋₁₀ aryloxy” or “C₆₋₁₀ arylthio” and the like, including but not limited to phenyloxy.

An “aralkyl” or “arylalkyl” is an aryl group connected, as a substituent, via an alkylene group, such “C₇₋₁₄ aralkyl” and the like, including but not limited to benzyl, 2-phenylethyl, 3-phenylpropyl, and naphthylalkyl. In some cases, the alkylene group is a lower alkylene group (i.e., a C₁₋₄ alkylene group).

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. In some embodiments, the heteroaryl group has 5 to 10 ring members or 5 to 7 ring members. The heteroaryl group may be designated as “5-7 membered heteroaryl,” “5-10 membered heteroaryl,” or similar designations. In various embodiments, a heteroaryl contains from 1 to 4 heteroatoms, from 1 to 3 heteroatoms, from 1 to 2 heteroatoms, or 1 heteroatom. For example, in various embodiments, a heteroaryl contains 1 to 4 nitrogen atoms, 1 to 3 nitrogen atoms, 1 to 2 nitrogen atoms, 2 nitrogen atoms and 1 sulfur or oxygen atom, 1 nitrogen atom and 1 sulfur or oxygen atom, or 1 sulfur or oxygen atom. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, triazinyl, quinolinyl, isoquinlinyl, benzimidazolyl, benzoxazolyl, benzothiazolyl, indolyl, isoindolyl, and benzothienyl.

A “heteroaralkyl” or “heteroarylalkyl” is heteroaryl group connected, as a substituent, via an alkylene group. Examples include but are not limited to 2-thienylmethyl, 3-thienylmethyl, furylmethyl, thienylethyl, pyrrolylalkyl, pyridylalkyl, isoxazollylalkyl, and imidazolylalkyl. In some cases, the alkylene group is a lower alkylene group (i.e., a C₁₋₄ alkylene group).

As used herein, “carbocyclyl” means a non-aromatic cyclic ring or ring system containing only carbon atoms in the ring system backbone. When the carbocyclyl is a ring system, two or more rings may be joined together in a fused, bridged or spiro-connected fashion. Carbocyclyls may have any degree of saturation provided that at least one ring in a ring system is not aromatic. Thus, carbocyclyls include cycloalkyls, cycloalkenyls, and cycloalkynyls. The carbocyclyl group may have 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group could also be a carbocyclyl having 3 to 6 carbon atoms. The carbocyclyl group may be designated as “C₃₋₆ carbocyclyl” or similar designations. Examples of carbocyclyl rings include, but are not limited to, cyclopropyl, cyclobutyl, cyclopentyl, cyclohexyl, cyclohexenyl, 2,3-dihydro-indene, bicycle[2.2.2]octanyl, adamantyl, and spiro[4.4]nonanyl.

A “(carbocyclyl)alkyl” is a carbocyclyl group connected, as a substituent, via an alkylene group, such as “C₄₋₁₀ (carbocyclyl)alkyl” and the like, including but not limited to, cyclopropylmethyl, cyclobutylmethyl, cyclopropylethyl, cyclopropylbutyl, cyclobutylethyl, cyclopropylisopropyl, cyclopentylmethyl, cyclopentylethyl, cyclohexylmethyl, cyclohexylethyl, cycloheptylmethyl, and the like. In some cases, the alkylene group is a lower alkylene group.

As used herein, “cycloalkyl” means a fully saturated carbocyclyl ring or ring system. Examples include cyclopropyl, cyclobutyl, cyclopentyl, and cyclohexyl.

As used herein, “cycloalkenyl” means a carbocyclyl ring or ring system having at least one double bond, wherein no ring in the ring system is aromatic. An example is cyclohexenyl.

As used herein, “heterocyclyl” means a non-aromatic cyclic ring or ring system containing at least one heteroatom in the ring backbone. Heterocyclyls may be joined together in a fused, bridged or spiro-connected fashion. Heterocyclyls may have any degree of saturation provided that at least one ring in the ring system is not aromatic. The heteroatom(s) may be present in either a non-aromatic or aromatic ring in the ring system. The heterocyclyl group may have 3 to 20 ring members (i.e., the number of atoms making up the ring backbone, including carbon atoms and heteroatoms), although the present definition also covers the occurrence of the term “heterocyclyl” where no numerical range is designated. The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations.

In various embodiments, a heterocyclyl contains from 1 to 4 heteroatoms, from 1 to 3 heteroatoms, from 1 to 2 heteroatoms, or 1 heteroatom. For example, in various embodiments, a heterocyclyl contains 1 to 4 nitrogen atoms, 1 to 3 nitrogen atoms, 1 to 2 nitrogen atoms, 2 nitrogen atoms and 1 sulfur or oxygen atom, 1 nitrogen atom and 1 sulfur or oxygen atom, or 1 sulfur or oxygen atom. In preferred six membered monocyclic heterocyclyls, the heteroatom(s) are selected from one up to three of O, N or S, and in preferred five membered monocyclic heterocyclyls, the heteroatom(s) are selected from one or two heteroatoms selected from O, N, or S. Examples of heterocyclyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline.

A “(heterocyclyl)alkyl” is a heterocyclyl group connected, as a substituent, via an alkylene group. Examples include, but are not limited to, imidazolinylmethyl and indolinylethyl.

As used herein, “acyl” refers to —C(═O)R, wherein R is hydrogen, C₁₋₆ alkyl, C₂₋₆ alkenyl, C₂₋₆ alkynyl, C₃₋₇ carbocyclyl, aryl, 5-10 membered heteroaryl, and 5-10 membered heterocyclyl, as defined herein. Non-limiting examples include formyl, acetyl, propanoyl, benzoyl, and acryl.

An “O-carboxy” group refers to a “—OC(═O)R” group in which R is selected from hydrogen, C₁₋₆ alkyl, C₂₋₆ alkenyl, C₂₋₆ alkynyl, C₃₋₇ carbocyclyl, aryl, 5-10 membered heteroaryl, and 5-10 membered heterocyclyl, as defined herein.

A “C-carboxy” group refers to a “—C(═O)OR” group in which R is selected from hydrogen, C₁₋₆ alkyl, C₂₋₆ alkenyl, C₂₋₆ alkynyl, C₃₋₇ carbocyclyl, aryl, 5-10 membered heteroaryl, and 5-10 membered heterocyclyl, as defined herein. A non-limiting example includes carboxyl (i.e., —C(═O)OH).

A “cyano” group refers to a “—CN” group.

A “cyanato” group refers to an “—OCN” group.

An “isocyanato” group refers to a “—NCO” group.

A “thiocyanato” group refers to a “—SCN” group.

An “isothiocyanato” group refers to an “—NCS” group.

A “sulfinyl” group refers to an “—S(═O)R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

A “sulfonyl” group refers to an “—SO₂R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “S-sulfonamido” group refers to a “—SO₂NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-sulfonamido” group refers to a “—N(R_{A})SO₂R_{B}” group in which R_{A} and R_{b} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “O-carbamyl” group refers to a “—OC(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-carbamyl” group refers to an “—N(R_{A})OC(═O)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “O-thiocarbamyl” group refers to a “—OC(═S)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-thiocarbamyl” group refers to an “—N(R_{A})OC(═S)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

A “C-amido” group refers to a “—C(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-amido” group refers to a “—N(R_{A})C(═O)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “amino” group refers to a “—NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “aminoalkyl” group refers to an amino group connected via an alkylene group.

An “alkoxyalkyl” group refers to an alkoxy group connected via an alkylene group, such as a “C₂₋₈ alkoxyalkyl” and the like.

As used herein, a “natural amino acid side chain” refers to the side-chain substituent of a naturally occurring amino acid. Naturally occurring amino acids have a substituent attached to the α-carbon. Naturally occurring amino acids include Arginine, Lysine, Aspartic acid, Glutamic acid, Glutamine, Asparagine, Histidine, Serine, Threonine, Tyrosine, Cysteine, Methionine, Tryptophan, Alanine, Isoleucine, Leucine, Phenylalanine, Valine, Proline, and Glycine.

As used herein, a “non-natural amino acid side chain” refers to the side-chain substituent of a non-naturally occurring amino acid. Non-natural amino acids include β-amino acids (β³ and β²), Homo-amino acids, Proline and Pyruvic acid derivatives, 3-substituted Alanine derivatives, Glycine derivatives, Ring-substituted Phenylalanine and Tyrosine Derivatives, Linear core amino acids and N-methyl amino acids. Exemplary non-natural amino acids are available from Sigma-Aldridge, listed under “unnatural amino acids & derivatives.” See also, Travis S. Young and Peter G. Schultz, “Beyond the Canonical 20 Amino Acids: Expanding the Genetic Lexicon,” J. Biol. Chem. 2010 285: 11039-11044, which is incorporated by reference in its entirety.

As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more substitutents independently selected from C₁-C₆ alkyl, C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 5-10 membered heterocyclyl (optionally substituted with halo, C₁-C₆ alkyl, C₁-C₆ alkoxy, C₁-C₆ haloalkyl, and C₁-C₆ haloalkoxy), 5-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), aryl(C₁-C₆)alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with halo, C₁-C₆ alkyl, C₁-C₆ alkoxy, C₁-C₆ haloalkyl, and C₁-C₆ haloalkoxy), halo, cyano, hydroxy, C₁-C₆ alkoxy, C₁-C₆ alkoxy(C₁-C₆)alkyl (i.e., ether), aryloxy, sulfhydryl (mercapto), halo(C₁-C₆)alkyl (e.g., —CF₃), halo(C₁-C₆)alkoxy (e.g., —OCF₃), C₁-C₆ alkylthio, arylthio, amino, amino(C₁-C₆)alkyl, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, and oxo (═O). Wherever a group is described as “optionally substituted” that group can be substituted with the above substituents.

In some embodiments, substituted group(s) is (are) substituted with one or more substituent(s) individually and independently selected from C₁-C₄ alkyl, amino, hydroxy, and halogen.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene” or “alkenylene.”

When two R groups are said to form a ring (e.g., a carbocyclyl, heterocyclyl, aryl, or heteroaryl ring) “together with the atom to which they are attached,” it is meant that the collective unit of the atom and the two R groups are the recited ring. The ring is not otherwise limited by the definition of each R group when taken individually. For example, when the following substructure is present:

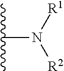

and R¹ and R² are defined as selected from the group consisting of hydrogen and alkyl, or R¹ and R² together with the nitrogen to which they are attached form a heterocyclyl, it is meant that R¹ and R² can be selected from hydrogen or alkyl, or alternatively, the substructure has structure:

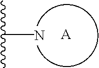

where ring A is a heterocyclyl ring containing the depicted nitrogen.

Similarly, when two “adjacent” R groups are said to form a ring “together with the atoms to which they are attached,” it is meant that the collective unit of the atoms, intervening bonds, and the two R groups are the recited ring. For example, when the following substructure is present:

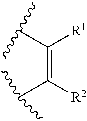

and R¹ and R² are defined as selected from the group consisting of hydrogen and alkyl, or R¹ and R² together with the atoms to which they are attached form an aryl or carbocyclyl, it is meant that R¹ and R² can be selected from hydrogen or alkyl, or alternatively, the substructure has structure:

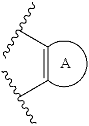

where A is an aryl ring or a carbocyclyl containing the depicted double bond.

Wherever a substituent is depicted as a di-radical (i.e., has two points of attachment to the rest of the molecule), it is to be understood that the substituent can be attached in any directional configuration unless otherwise indicated. Thus, for example, a substituent depicted as -AE- or

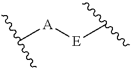

includes the substituent being oriented such that the A is attached at the leftmost attachment point of the molecule as well as the case in which A is attached at the rightmost attachment point of the molecule.

As used herein, the substructure:

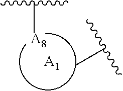

means that the A₈ atom can be in any ring atom position within the ring or ring system A₁. The substructure:

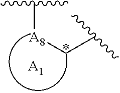

means that the A₈ atom is in the ring atom position immediately adjacent (i.e., alpha) to the point of attachment indicated by *.

As used herein, “isosteres” of a chemical group are other chemical groups that exhibit the same or similar properties. For example, tetrazole is an isostere of carboxylic acid because it mimics the properties of carboxylic acid even though they both have very different molecular formulae. Tetrazole is one of many possible isosteric replacements for carboxylic acid. Other carboxylic acid isosteres contemplated include —SO₃H, —SO₂HNR, —PO₂(R)₂, —PO₃(R)₂, —CONHNHSO₂R, —COHNSO₂R, and —CONRCN, where R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein. In addition, carboxylic acid isosteres can include 5-7 membered carbocycles or heterocycles containing any combination of CH₂, O, S, or N in any chemically stable oxidation state, where any of the atoms of said ring structure are optionally substituted in one or more positions. The following structures are non-limiting examples of carbocyclic and heterocyclic isosteres contemplated. The atoms of said ring structure may be optionally substituted at one or more positions with R as defined above.

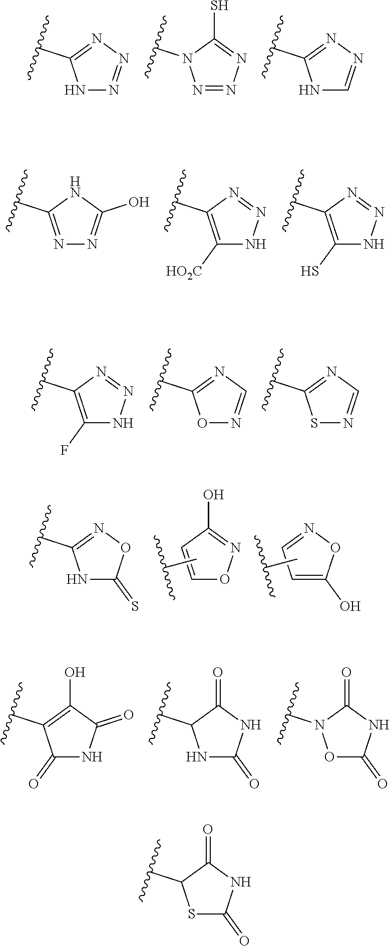

It is also contemplated that when chemical substituents are added to a carboxylic isostere, the compound retains the properties of a carboxylic isostere. It is contemplated that when a carboxylic isostere is optionally substituted with one or more moieties selected from R as defined above, then the substitution and substitution position is selected such that it does not eliminate the carboxylic acid isosteric properties of the compound. Similarly, it is also contemplated that the placement of one or more R substituents upon a carbocyclic or heterocyclic carboxylic acid isostere is not a substitution at one or more atom(s) that maintain(s) or is/are integral to the carboxylic acid isosteric properties of the compound, if such substituent(s) would destroy the carboxylic acid isosteric properties of the compound.

Other carboxylic acid isosteres not specifically exemplified in this specification are also contemplated.

The term “agent” or “test agent” includes any substance, molecule, element, compound, entity, or a combination thereof. It includes, but is not limited to, e.g., protein, polypeptide, peptide or mimetic, small organic molecule, polysaccharide, polynucleotide, and the like. It can be a natural product, a synthetic compound, or a chemical compound, or a combination of two or more substances. Unless otherwise specified, the terms “agent”, “substance”, and “compound” are used interchangeably herein.

The term “analog” is used herein to refer to a molecule that structurally resembles a reference molecule but which has been modified in a targeted and controlled manner, by replacing a specific substituent of the reference molecule with an alternate substituent. Compared to the reference molecule, an analog would be expected, by one skilled in the art, to exhibit the same, similar, or improved utility. Synthesis and screening of analogs, to identify variants of known compounds having improved characteristics (such as higher binding affinity for a target molecule) is an approach that is well known in pharmaceutical chemistry.

### Methods of Treatment

In some embodiments, the compounds disclosed herein are calpain inhibitors. In some embodiments, the compounds can effectively act as CAPN1, CAPN2, and/or CAPN9 inhibitors. Some embodiments provide pharmaceutical compositions comprising one or more compounds disclosed herein and a pharmaceutically acceptable excipient.

Some embodiments provide a method for treating liver fibrosis with an effective amount of one or more compounds as disclosed herein.

Some embodiments provide a method for treating primary sclerosing cholangitis with an effective amount of one or more compounds as disclosed herein. Some embodiments provide a method for treating primary biliary cholangitis with an effective amount of one or more compounds as disclosed herein. Some embodiments provide a method for treating non-alcoholic fatty liver disease with an effective amount of one or more compounds as disclosed herein. Some embodiments provide a method for treating non-alcoholic steatohepatitis with an effective amount of one or more compounds as disclosed herein.

Some embodiments provide a method for treating, liver cirrhosis with an effective amount of one or more compounds as disclosed herein. In some embodiments, the liver cirrhosis is caused by one or more of the conditions selected from the group consisting of alcoholic liver disease, alpha-1 antitrypsin deficiency, autoimmune hepatitis, celiac disease, chronic viral hepatitis, hemochromatosis, idiopathic portal fibrosis, and Wilson disease.

In some embodiments, the subject is a mammal. In some specific embodiments, the subject is a human.

Further embodiments include administering a combination of compounds to a subject in need thereof. A combination can include a compound, composition, pharmaceutical composition described herein with an additional medicament.

Some embodiments include co-administering a compound, composition, and/or pharmaceutical composition described herein, with an additional medicament. By “co-administration,” it is meant that the two or more agents may be found in the patient's bloodstream at the same time, regardless of when or how they are actually administered. In one embodiment, the agents are administered simultaneously. In one such embodiment, administration in combination is accomplished by combining the agents in a single dosage form. In another embodiment, the agents are administered sequentially. In one embodiment the agents are administered through the same route, such as orally. In another embodiment, the agents are administered through different routes, such as one being administered orally and another being administered i.v.

Some embodiments include a combination of the compounds, compositions and/or pharmaceutical compositions described herein with an additional agent, such as anti-inflammatories including glucocorticoids, analgesics (e.g. ibuprofen), aspirin, and agents that modulate a Th2-immune response, immunosuppressants including methotrexate, mycophenolate, cyclophosphamide, cyclosporine, thalidomide, pomalidomide, leflunomide, hydroxychloroquine, azathioprine, soluble bovine cartilage, vasodilators including endothelin receptor antagonists, prostacyclin analogues, nifedipine, and sildenafil, IL-6 receptor antagonists, selective and non-selective tyrosine kinase inhibitors, Wnt-pathway modulators, PPAR activators, caspase-3 inhibitors, LPA receptor antagonists, B cell depleting agents, CCR2 antagonists, pirfenidone, cannabinoid receptor agonists, ROCK inhibitors, miRNA-targeting agents, toll-like receptor antagonists, CTGF-targeting agents, NADPH oxidase inhibitors, tryptase inhibitors, TGFD inhibitors, relaxin receptor agonists, and autologous adipose derived regenerative cells.

In some embodiments provided herein, the calpain inhibitor described herein may be administered in combination with one or more additional agents selected from the group consisting of a VAP-1 inhibitor, an ASBT Inhibitor, a dual CCR2/5 antagonist, an anti-cholestatic bile acid, a FXR agonist, a FGFR1c/4 agonist, mesenchymal stem cell (MSC) cell therapy, a CCL24 Inhibitor, and a CCL11 inhibitor. In some embodiments, the calpain inhibitor may be used in combination with one or more additional aforementioned agents in a method of treating primary sclerosing cholangitis (PSC), the method comprising administering the calpain inhibitor in combination with one or more additional aforementioned agents to a subject in need thereof.

In some embodiments provided herein, the calpain inhibitor described herein may be administered in combination with one or more additional agents selected from the group consisting of obeticholic acid, elafibranor, cenicriviroc, selonsertib, a niacin receptor agonist, a SGLT2 inhibitor, a VAP-1 inhibitor, a FGF21 mimetic, a adenosine A3 receptor agonist, a mTOT modulator, a FXR agonist, a galectin-3 inhibitor, an ABCA1 activator, a SCD1 inhibitor, an ACC inhibitor, a Type I NK T-cell inhibitor, a pan-PPAR agonist, a DGAT2 inhibitor, a PPARalpha agonist, a thyroid hormone R-b agonist, a 5-LO/LT inhibitor, a mineralocorticoid receptor antagonist, a FGF19 mimic, a caspase inhibitor, a GLP-1R agonist, a SIRT1/AMP agonist, an ACC inhibitor, a ketohexokinase inhibitor, a GLP-1R agonist, an ASBT inhibitor, a DGAT2/CYP2E1 inhibitor, a TLR4 antagonist, a thyroid hormone R-b agonist, a IFN-gamma receptor antagonism, a CB1 antagonist, a FGF21 ligand, a P2Y13 receptor agonist, a CCL24 inhibitor, a MCH receptor-1 antagonist, aPPARalpha, delta agonist, a DPP-4 inhibitor, aLXR antagonist, a GLP1R agonist, an eotaxin-1 inhibitor, a beta-klotho/FGFR1c agonist, a LOXL2 Inhibitor, an AMPK activator, a miR-103/107 inhibitor, an inflammasome inhibitor, a CD3 antagonist, and a cathepsin B inhibitor. In some embodiments, the calpain inhibitor may be used in combination with one or more additional aforementioned agents in a method of treating non-alcoholic steatohepatitis (NASH), the method comprising administering the calpain inhibitor in combination with one or more additional aforementioned agents to a subject in need thereof.

### Administration and Pharmaceutical Compositions

The compounds are administered at a therapeutically effective dosage. While human dosage levels have yet to be optimized for the compounds described herein, generally, a daily dose may be from about 0.25 mg/kg to about 120 mg/kg or more of body weight, from about 0.5 mg/kg or less to about 70 mg/kg, from about 1.0 mg/kg to about 50 mg/kg of body weight, or from about 1.5 mg/kg to about 10 mg/kg of body weight. Thus, for administration to a 70 kg person, the dosage range would be from about 17 mg per day to about 8000 mg per day, from about 35 mg per day or less to about 7000 mg per day or more, from about 70 mg per day to about 6000 mg per day, from about 100 mg per day to about 5000 mg per day, or from about 200 mg to about 3000 mg per day. The amount of active compound administered will, of course, be dependent on the subject and disease state being treated, the severity of the affliction, the manner and schedule of administration and the judgment of the prescribing physician.

Administration of the compounds disclosed herein or the pharmaceutically acceptable salts thereof can be via any of the accepted modes of administration for agents that serve similar utilities including, but not limited to, orally, subcutaneously, intravenously, intranasally, topically, transdermally, intraperitoneally, intramuscularly, intrapulmonarilly, vaginally, rectally, or intraocularly. Oral and parenteral administrations are customary in treating the indications that are the subject of the preferred embodiments.

The compounds useful as described above can be formulated into pharmaceutical compositions for use in treatment of these conditions. Standard pharmaceutical formulation techniques are used, such as those disclosed in Remington's The Science and Practice of Pharmacy, 21st Ed., Lippincott Williams & Wilkins (2005), incorporated by reference in its entirety. Accordingly, some embodiments include pharmaceutical compositions comprising: (a) a safe and therapeutically effective amount of a compound described herein (including enantiomers, diastereoisomers, tautomers, polymorphs, and solvates thereof), or pharmaceutically acceptable salts thereof; and (b) a pharmaceutically acceptable carrier, diluent, excipient or combination thereof.

In addition to the selected compound useful as described above, come embodiments include compositions containing a pharmaceutically-acceptable carrier. The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the TWEENS; wetting agents, such sodium lauryl sulfate; coloring agents; flavoring agents; tableting agents, stabilizers; antioxidants; preservatives; pyrogen-free water; isotonic saline; and phosphate buffer solutions.

The choice of a pharmaceutically-acceptable carrier to be used in conjunction with the subject compound is basically determined by the way the compound is to be administered.

The compositions described herein are preferably provided in unit dosage form. As used herein, a “unit dosage form” is a composition containing an amount of a compound that is suitable for administration to an animal, preferably mammal subject, in a single dose, according to good medical practice. The preparation of a single or unit dosage form however, does not imply that the dosage form is administered once per day or once per course of therapy. Such dosage forms are contemplated to be administered once, twice, thrice or more per day and may be administered as infusion over a period of time (e.g., from about 30 minutes to about 2-6 hours), or administered as a continuous infusion, and may be given more than once during a course of therapy, though a single administration is not specifically excluded. The skilled artisan will recognize that the formulation does not specifically contemplate the entire course of therapy and such decisions are left for those skilled in the art of treatment rather than formulation.

The compositions useful as described above may be in any of a variety of suitable forms for a variety of routes for administration, for example, for oral, nasal, rectal, topical (including transdermal), ocular, intracerebral, intracranial, intrathecal, intra-arterial, intravenous, intramuscular, or other parental routes of administration. The skilled artisan will appreciate that oral and nasal compositions comprise compositions that are administered by inhalation, and made using available methodologies. Depending upon the particular route of administration desired, a variety of pharmaceutically-acceptable carriers well-known in the art may be used. Pharmaceutically-acceptable carriers include, for example, solid or liquid fillers, diluents, hydrotropies, surface-active agents, and encapsulating substances. Optional pharmaceutically-active materials may be included, which do not substantially interfere with the inhibitory activity of the compound. The amount of carrier employed in conjunction with the compound is sufficient to provide a practical quantity of material for administration per unit dose of the compound. Techniques and compositions for making dosage forms useful in the methods described herein are described in the following references, all incorporated by reference herein: Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

Various oral dosage forms can be used, including such solid forms as tablets, capsules, granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically-acceptable carrier suitable for the preparation of unit dosage forms for peroral administration is well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical, and can be readily made by a person skilled in the art.

Peroral compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes and shellac.

Compositions described herein may optionally include other drug actives.

Other compositions useful for attaining systemic delivery of the subject compounds include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

A liquid composition, which is formulated for topical ophthalmic use, is formulated such that it can be administered topically to the eye. The comfort should be maximized as much as possible, although sometimes formulation considerations (e.g. drug stability) may necessitate less than optimal comfort. In the case that comfort cannot be maximized, the liquid should be formulated such that the liquid is tolerable to the patient for topical ophthalmic use. Additionally, an ophthalmically acceptable liquid should either be packaged for single use, or contain a preservative to prevent contamination over multiple uses.

For ophthalmic application, solutions or medicaments are often prepared using a physiological saline solution as a major vehicle. Ophthalmic solutions should preferably be maintained at a comfortable pH with an appropriate buffer system. The formulations may also contain conventional, pharmaceutically acceptable preservatives, stabilizers and surfactants.

Preservatives that may be used in the pharmaceutical compositions disclosed herein include, but are not limited to, benzalkonium chloride, PHMB, chlorobutanol, thimerosal, phenylmercuric, acetate and phenylmercuric nitrate. A useful surfactant is, for example, Tween 80. Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. For many compositions, the pH will be between 4 and 9. Accordingly, buffers include acetate buffers, citrate buffers, phosphate buffers and borate buffers. Acids or bases may be used to adjust the pH of these formulations as needed.

In a similar vein, an ophthalmically acceptable antioxidant includes, but is not limited to, sodium metabisulfite, sodium thiosulfate, acetylcysteine, butylated hydroxyanisole and butylated hydroxytoluene.

Other excipient components, which may be included in the ophthalmic preparations, are chelating agents. A useful chelating agent is edetate disodium, although other chelating agents may also be used in place or in conjunction with it.

For topical use, creams, ointments, gels, solutions or suspensions, etc., containing the compound disclosed herein are employed. Topical formulations may generally be comprised of a pharmaceutical carrier, co-solvent, emulsifier, penetration enhancer, preservative system, and emollient.

For intravenous administration, the compounds and compositions described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HCl, and citric acid. In various embodiments, the pH of the final composition ranges from 2 to 8, or preferably from 4 to 7. Antioxidant excipients may include sodium bisulfite, acetone sodium bisulfite, sodium formaldehyde, sulfoxylate, thiourea, and EDTA. Other non-limiting examples of suitable excipients found in the final intravenous composition may include sodium or potassium phosphates, citric acid, tartaric acid, gelatin, and carbohydrates such as dextrose, mannitol, and dextran. Further acceptable excipients are described in Powell, et al., Compendium of Excipients for Parenteral Formulations, PDA J Pharm Sci and Tech 1998, 52 238-311 and Nema et al., Excipients and Their Role in Approved Injectable Products: Current Usage and Future Directions, PDA J Pharm Sci and Tech 2011, 65 287-332, both of which are incorporated herein by reference in their entirety. Antimicrobial agents may also be included to achieve a bacteriostatic or fungistatic solution, including but not limited to phenylmercuric nitrate, thimerosal, benzethonium chloride, benzalkonium chloride, phenol, cresol, and chlorobutanol.

The compositions for intravenous administration may be provided to caregivers in the form of one more solids that are reconstituted with a suitable diluent such as sterile water, saline or dextrose in water shortly prior to administration. In other embodiments, the compositions are provided in solution ready to administer parenterally. In still other embodiments, the compositions are provided in a solution that is further diluted prior to administration. In embodiments that include administering a combination of a compound described herein and another agent, the combination may be provided to caregivers as a mixture, or the caregivers may mix the two agents prior to administration, or the two agents may be administered separately.

The actual dose of the active compounds described herein depends on the specific compound, and on the condition to be treated; the selection of the appropriate dose is well within the knowledge of the skilled artisan.

The compounds and compositions described herein, if desired, may be presented in a pack or dispenser device containing one or more unit dosage forms containing the active ingredient. Such a pack or device may, for example, comprise metal or plastic foil, such as a blister pack, or glass, and rubber stoppers such as in vials. The pack or dispenser device may be accompanied by instructions for administration. Compounds and compositions described herein are formulated in a compatible pharmaceutical carrier may also be prepared, placed in an appropriate container, and labeled for treatment of an indicated condition.

The amount of the compound in a formulation can vary within the full range employed by those skilled in the art. Typically, the formulation will contain, on a weight percent (wt %) basis, from about 0.01 99.99 wt % of a compound of the present technology based on the total formulation, with the balance being one or more suitable pharmaceutical excipients. Preferably, the compound is present at a level of about 1 80 wt %. Representative pharmaceutical formulations are described below.

### FORMULATION EXAMPLES

The following are representative pharmaceutical formulations containing a compound of Formula I.

### Formulation Example 1—Tablet formulation

The following ingredients are mixed intimately and pressed into single scored tablets.

| Ingredient | Quantity per tablet, mg |
|---|---|
| Compounds disclosed herein | 400 |
| cornstarch | 50 |
| croscarmellose sodium | 25 |
| lactose | 120 |
| magnesium stearate | 5 |

### Formulation Example 2—Capsule formulation

The following ingredients are mixed intimately and loaded into a hard-shell gelatin capsule.

| Ingredient | Quantity per capsule, mg |
|---|---|
| Compounds disclosed herein | 200 |
| lactose, spray-dried | 148 |
| magnesium stearate | 2 |

### Formulation Example 3—Suspension formulation

The following ingredients are mixed to form a suspension for oral administration.

| Ingredient | Amount | |
|---|---|---|
| Compounds disclosed herein | 1.0 | g |
| fumaric acid | 0.5 | g |
| sodium chloride | 2.0 | g |
| methyl paraben | 0.15 | g |
| propyl paraben | 0.05 | g |
| granulated sugar | 25.0 | g |
| sorbitol (70% solution) | 13.00 | g |
| Veegum K (Vanderbilt Co.) | 1.0 | g |
| flavoring | 0.035 | mL |
| colorings | 0.5 | mg |

| | |
|---|---|
| distilled water | q.s. to 100 mL |

### Formulation Example 4—Injectable formulation

The following ingredients are mixed to form an injectable formulation.

| Ingredient | Amount |
|---|---|
| Compounds disclosed herein | 0.2 mg-20 mg |
| sodium acetate buffer solution, 0.4M | 2.0 mL |
| HCl (1N) or NaOH (1N) | q.s. to suitable pH |
| water (distilled, sterile) | q.s. to 20 mL |

### Formulation Example 5—Suppository Formulation

A suppository of total weight 2.5 g is prepared by mixing the compound of the present technology with Witepsol® H-15 (triglycerides of saturated vegetable fatty acid; Riches-Nelson, Inc., New York), and has the following composition:

| Ingredient | Amount |
|---|---|
| Compounds disclosed herein | 500 mg |
| Witepsol ® H-15 | balance |

### Compounds

In some embodiments, the calpain inhibitor may be selected from a compound having the structure of the Formula I:

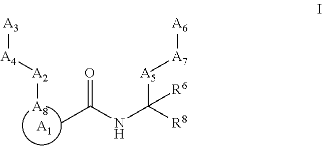

or a pharmaceutically acceptable salt thereof, wherein:

A₁ is selected from the group consisting of optionally substituted 5-10 membered heterocyclyl provided the 5-10 membered heterocyclyl is not substituted with oxo, optionally substituted 5-, 8-, or 9-membered heteroaryl, and optionally substituted C₃₋₁₀ carbocyclyl;

A₂ is selected from the group consisting of optionally substituted 3-10 membered heterocyclyl, optionally substituted C₆₋₁₀ aryl, optionally substituted 5-10 membered heteroaryl, and optionally substituted C₃₋₁₀ carbocyclyl, —CR₂—, —S—, —S(═O)—, —SO₂—, —O—, —C(═S)—, —C(═O)—, —NR—, —CH═CH—, —C≡C—, —OC(O)NH—, —NHC(O)NH—, —NHC(O)O—, —NHC(O)—, —NHC(S)NH—, —NHC(S)O—, —NHC(S)—, and single bond;

A₄ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₄ alkyl, —(CR₂)ₙ—S—(CR₂)ₙ—, —(CR₂)ₙ—S(═O)—(CR₂)ₙ—, —(CR₂)ₙ—SO₂—(CR₂)ₙ—, —(CR₂)ₙ—O—(CR₂)ₙ—, —(CR₂)ₙ—C(═S)—(CR₂)ₙ—, —(CR₂)ₙ—C(═O)—(CR₂)ₙ—, —(CR₂)ₙ—NR—(CR₂)ₙ—, —(CR₂)ₙ—CH═CH—(CR₂)ₙ—, —(CR₂)ₙ—OC(O)NH—(CR₂)ₙ—, —(CR₂)ₙ—NHC(O)NH—(CR₂)ₙ—, —(CR₂)ₙ—NHC(O)O—(CR₂)ₙ—, —(CR₂)ₙ—NHC(O)—(CR₂)ₙ—, —(CR₂)ₙ—NHC(S)NH—(CR₂)ₙ—, —(CR₂)ₙ—NHC(S)O—(CR₂)ₙ—, —(CR₂)ₙ—NHC(S)—(CR₂)ₙ—, and single bond;

when A₂ and A₄ are single bond, A₃ is directly attached to A₈;

A₃ is selected from the group consisting of optionally substituted C₆₋₁₀ aryl, optionally substituted 5-10 membered heteroaryl, optionally substituted 3-10 membered heterocyclyl, and optionally substituted C₃₋₁₀ carbocyclyl, or if A₂ is selected from optionally substituted 3-10 membered heterocyclyl, optionally substituted C₆₋₁₀ aryl, optionally substituted 5-10 membered heteroaryl, and optionally substituted C₃₋₁₀ carbocyclyl, then A₃ is selected from the group consisting of hydrogen, optionally substituted C₆₋₁₀ aryl, optionally substituted 5-10 membered heteroaryl, optionally substituted 3-10 membered heterocyclyl, optionally substituted C₃₋₁₀ carbocyclyl, —C≡CH, and optionally substituted 2- to 5-membered polyethylene glycol;

A₅ is selected from the group consisting of optionally substituted 3-10 membered heterocyclyl, optionally substituted C₆₋₁₀ aryl, optionally substituted 5-10 membered heteroaryl, optionally substituted C₃₋₁₀ carbocyclyl, optionally substituted C₁₋₈ alkyl, —S—, —S(═O)—, —SO₂—, —O—, —C(═S)—, —C(═O)—, —NR—, —CH═CH—, —OC(O)NH—, —NHC(O)NH—, —NHC(O)O—, —NHC(O)—, —NHC(S)NH—, —NHC(S)O—, —NHC(S)—, and single bond;

A₆ is selected from the group consisting of optionally substituted C₆₋₁₀ aryl, optionally substituted 5-10 membered heteroaryl, optionally substituted 3-10 membered heterocyclyl, and optionally substituted C₃₋₁₀ carbocyclyl, optionally substituted C₁₋₈ alkyl, optionally substituted C₂₋₈ alkenyl, optionally substituted —O—C₁₋₆ alkyl, optionally substituted —O C₂₋₆ alkenyl, —OSO₂CF₃, and any natural or non-natural amino acid side chain;

A₇ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₈ alkyl, —S—, S(═O)—, —SO₂—, —O—, —C(═S)—, —C(═O)—, —NR—, —CH═CH—, —OC(O)NH—, —NHC(O)NH—, —NHC(O)O—, —NHC(O)—, —NHC(S)NH—, —NHC(S)O—, —NHC(S)—, and single bond;

when A₅ and A₇ are single bond, A₆ is directly attached to the carbon to which R⁸ is attached;

A₈ is a ring member of A₁ and selected from the group consisting of C, CH, and N;

R⁸ is selected from the group consisting of —COR¹, —CN, —CH═CHSO₂R, and —CH₂NO₂;

R¹ is selected from the group consisting of H, —OH, C₁₋₄ haloalkyl, —COOH, —CH₂NO₂, —C(═O)NOR, —NH₂, —CONR²R³, —CH(CH₃)═CH₂, —CH(CF₃)NR²R³, —C(F)═CHCH₂CH₃,

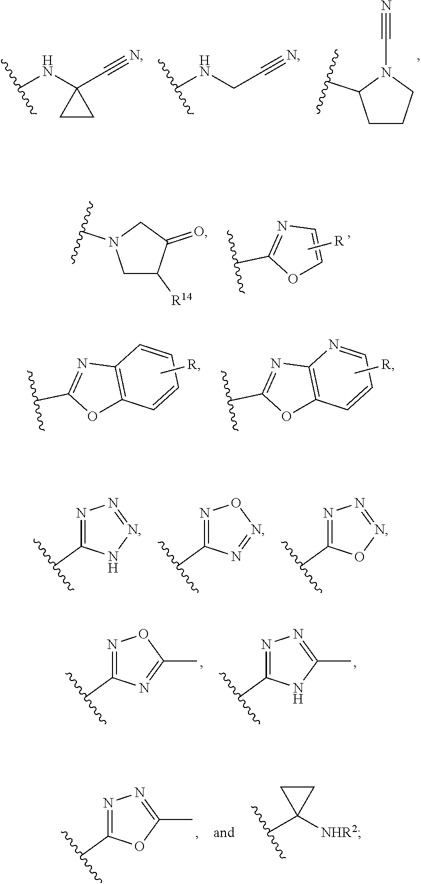

R¹⁴ is halo;

each R, R², and R³ are independently selected from —H, optionally substituted

C₁₋₄ alkyl, optionally substituted C₁₋₈ alkoxyalkyl, optionally substituted 2- to 5-membered polyethylene glycol, optionally substituted C₃₋₇ carbocyclyl, optionally substituted 5-10 membered heterocyclyl, optionally substituted C₆₋₁₀ aryl, and optionally substituted 5-10 membered heteroaryl; and

R⁶ is independently selected from —H and optionally substituted C₁₋₄ alkyl.

In some embodiments, the calpain inhibitor may be selected from a compound having the structure of Formula II:

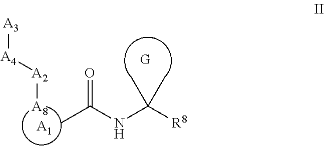

or a pharmaceutically acceptable salt thereof, wherein:

A₁ is selected from the group consisting of optionally substituted 5-10 membered heterocyclyl provided the 6-10-membered heterocyclyl is not substituted with oxo; optionally substituted 5-, 8-, or 9-membered heteroaryl; and optionally substituted C₃₋₁₀ carbocyclyl;

A₂ is selected from the group consisting of optionally substituted 3-10 membered heterocyclyl, optionally substituted C₆₋₁₀ aryl, optionally substituted 5-10 membered heteroaryl, optionally substituted C₃₋₁₀ carbocyclyl, —CR₂—, —S—, —S(═O)—, —SO₂—, —O—, —C(═S)—, —C(═O)—, —NR—, —CH═CH—, —C≡C—, —OC(O)NH—, —NHC(O)NH—, —NHC(O)O—, —NHC(O)—, —NHC(S)NH—, —NHC(S)O—, —NHC(S)—, and single bond;

A₄ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₄ alkyl, —(CR₂)ₙ—S—(CR₂)ₙ—, —(CR₂)ₙ—S(═O)—(CR₂)ₙ—, —(CR₂)ₙ—SO₂—(CR₂)ₙ—, —(CR₂)ₙ—O—(CR₂)ₙ—, —(CR₂)ₙ—C(═S)—(CR₂)ₙ—, —(CR₂)ₙ—C(═O)—(CR₂)ₙ—, —(CR₂)ₙ—NR—(CR₂)ₙ—, —(CR₂)ₙ—CH═CH—(CR₂)ₙ—, —(CR₂)ₙ—OC(O)NH—(CR₂)ₙ—, —(CR₂)ₙ—NHC(O)NH—(CR₂)ₙ—, —(CR₂)ₙ—NHC(O)O—(CR₂)ₙ—, —(CR₂)ₙ—NHC(O)—(CR₂)ₙ—, —(CR₂)ₙ—NHC(S)NH—(CR₂)ₙ—, —(CR₂)ₙ—NHC(S)O—(CR₂)ₙ—, —(CR₂)ₙ—NHC(S)—(CR₂)ₙ—, and single bond;

when A₂ and A₄ are single bond, A₃ is directly attached to A₈;

A₃ is selected from the group consisting of optionally substituted C₆₋₁₀ aryl, optionally substituted 5-10 membered heteroaryl, optionally substituted 3-10 membered heterocyclyl, and optionally substituted C₃₋₁₀ carbocyclyl, or if A₂ is selected from optionally substituted 3-10 membered heterocyclyl, optionally substituted C₆₋₁₀ aryl, optionally substituted 5-10 membered heteroaryl, and optionally substituted C₃₋₁₀ carbocyclyl, then A₃ is selected from the group consisting of hydrogen, optionally substituted C₆₋₁₀ aryl, optionally substituted 5-10 membered heteroaryl, optionally substituted 3-10 membered heterocyclyl, optionally substituted C₃₋₁₀ carbocyclyl, —C≡CH, and optionally substituted 2- to 5-membered polyethylene glycol;

G is an optionally substituted C₃ to C₇ carbocyclyl or an optionally substituted 4- to 7-membered heterocyclyl;

A₈ is a ring member of A₁ and is selected from the group consisting of C and N;

R⁸ is selected from the group consisting of —COR¹, —CN, —CH═CHSO₂R, —CH₂NO₂;

R¹ is selected from the group consisting of H, —OH, C₁₋₄ haloalkyl, —COOH, —CH₂NO₂, —C(═O)NOR, —NH₂, —CONR²R³, —CH(CH₃)═CH₂, —CH(CF₃)NR²R³, —C(F)═CHCH₂CH₃,

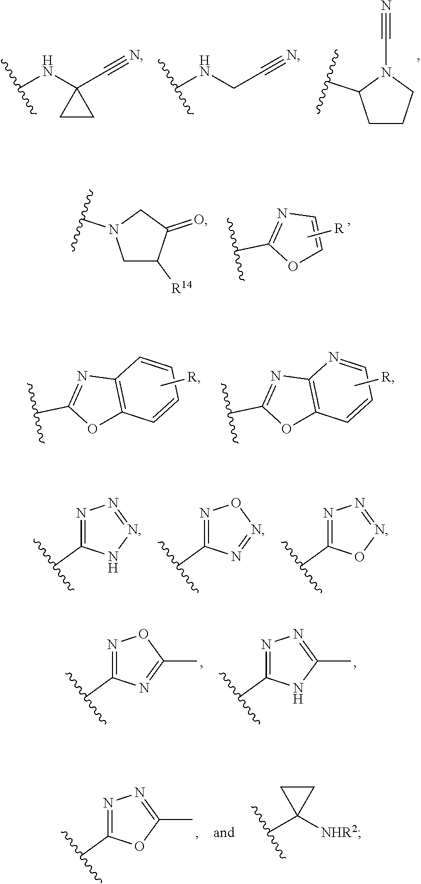

R¹⁴ is halo; and

each R, R², and R³ are independently selected from —H, optionally substituted C₁₋₄ alkyl, optionally substituted C₁₋₈ alkoxyalkyl, optionally substituted 2- to 5-membered polyethylene glycol, optionally substituted C₃₋₇ carbocyclyl, optionally substituted 5-10 membered heterocyclyl, optionally substituted C₆₋₁₀ aryl, optionally substituted C₆₋₁₀ aryl(C₁-C₆)alkyl, and optionally substituted 5-10 membered heteroaryl; R⁶ is independently selected from —H and optionally substituted C₁₋₄ alkyl; and each n is independently selected to be an integer from 0 to 3.

In some embodiments, the calpain inhibitor may be a compound having the structure of Formula III:

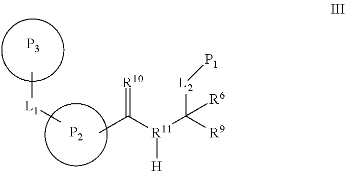

or a pharmaceutically acceptable salt thereof, wherein:

P₂ is an optionally substituted cyclic moiety having a size and configuration such that, upon binding of the compound to calpain 9, at least one atom of P₂ forms a non-polar interaction with, and is within 5 Å or less of, at least one calpain 9 P2 pocket moiety selected from the group consisting of Gly190, Phe233, Gly253, His254, and Ala255;

L₁ is a bond or a moiety consisting of from 1 to 25 atoms selected from the group consisting of carbon, oxygen, nitrogen, hydrogen, and sulfur;

P₃ is an optionally substituted cyclic moiety positioned by L₁ and having a size and configuration such that, upon binding of the compound to calpain 9, at least one atom of P₃ forms a non-polar interaction with, and is within 5 Å or less of, at least one calpain 9 P3 pocket moiety selected from the group consisting of Gly189, Gly190, Ser191, Thr236, and Gly253;

R¹⁰ is oxo and is positioned by P₂ such that, upon binding of the compound to calpain 9, R¹⁰ forms a polar interaction with, and is within 4 Å or less of, calpain 9 Gly190 amide;

R¹¹ is nitrogen and is positioned by the carbons to which it is bonded such that, upon binding of the compound to calpain 9, R¹¹ forms a polar interaction with, and is within 4 Å or less of, calpain 9 Gly253 carbonyl;

L₂ is a bond or a moiety consisting of from 1 to 25 atoms selected from the group consisting of carbon, oxygen, nitrogen, hydrogen, and sulfur;

P₁ is a moiety positioned by L₂ and having a size and configuration such that, upon binding of the compound to calpain 9, at least one atom of P₁ forms a non-polar interaction with, and is within 5 Å or less of, at least one calpain 9 P1 pocket moiety selected from the group consisting of Gly95, Lys188, Gly189, and Ser242;

R⁹ is a moiety positioned by the carbon to which it is attached such that, upon binding of the compound to calpain 9, at least one atom of R⁹ forms a polar interaction with, and is within 4 Å or less of, at least one calpain 9 moiety selected from the group consisting of Gln91, Cys97, and His254; and

R⁶ is selected from —H and optionally substituted C₁₋₄ alkyl.

In some embodiments, the calpain inhibitor can be selected from the group consisting of the compounds listed in Tables 1a and 1b below, or pharmaceutically acceptable salts thereof.

TABLE 1a

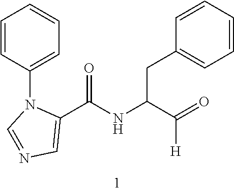 [SMILES: O=CC(Cc1ccccc1)NC(=O)c1cncn1-c1ccccc1]

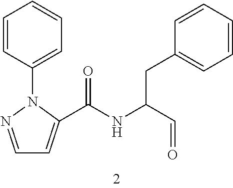 [SMILES: O=CC(Cc1ccccc1)NC(=O)c1ccnn1-c1ccccc1]

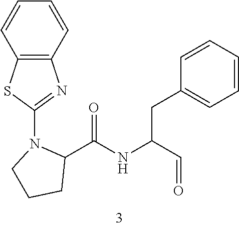 [SMILES: O=CC(Cc1ccccc1)NC(=O)C1CCCN1c1nc2ccccc2s1]

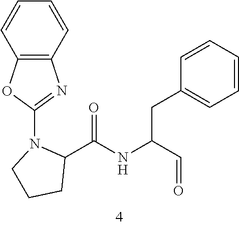 [SMILES: O=CC(Cc1ccccc1)NC(=O)C1CCCN1c1nc2ccccc2o1]

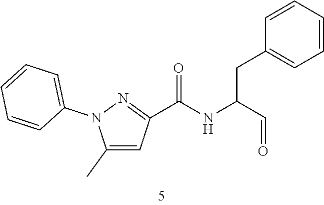 [SMILES: Cc1cc(C(=O)NC(C=O)Cc2ccccc2)nn1-c1ccccc1]

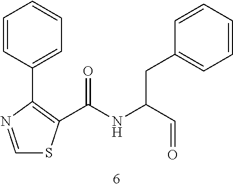 [SMILES: O=CC(Cc1ccccc1)NC(=O)c1scnc1-c1ccccc1]

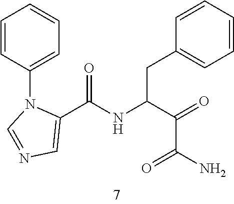 [SMILES: NC(=O)C(=O)C(Cc1ccccc1)NC(=O)c1cncn1-c1ccccc1]

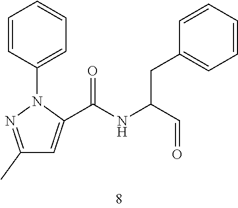 [SMILES: Cc1cc(C(=O)NC(C=O)Cc2ccccc2)n(-c2ccccc2)n1]

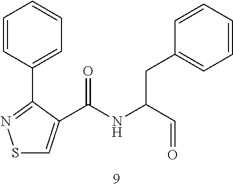 [SMILES: O=CC(Cc1ccccc1)NC(=O)c1csnc1-c1ccccc1]

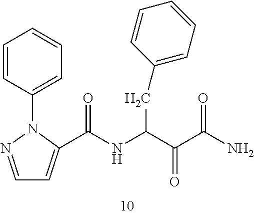 [SMILES: NC(=O)C(=O)C(Cc1ccccc1)NC(=O)c1ccnn1-c1ccccc1]

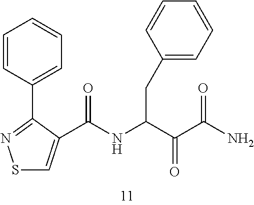 [SMILES: NC(=O)C(=O)C(Cc1ccccc1)NC(=O)c1csnc1-c1ccccc1]

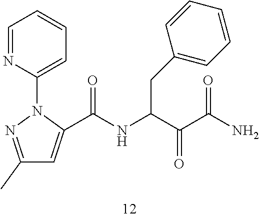 [SMILES: Cc1cc(C(=O)NC(Cc2ccccc2)C(=O)C(N)=O)n(-c2ccccn2)n1]

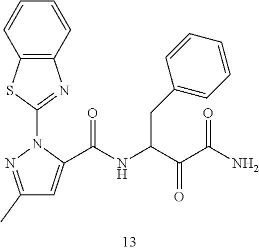 [SMILES: Cc1cc(C(=O)NC(Cc2ccccc2)C(=O)C(N)=O)n(-c2nc3ccccc3s2)n1]

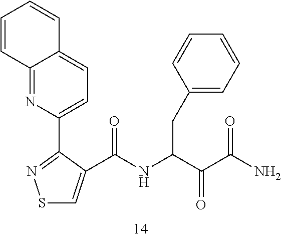 [SMILES: NC(=O)C(=O)C(Cc1ccccc1)NC(=O)c1csnc1-c1ccc2ccccc2n1]

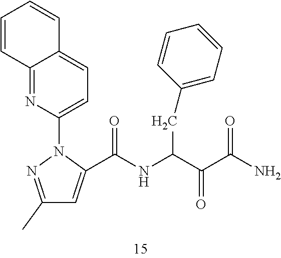 [SMILES: Cc1cc(C(=O)NC(Cc2ccccc2)C(=O)C(N)=O)n(-c2ccc3ccccc3n2)n1]

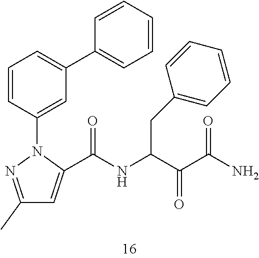 [SMILES: Cc1cc(C(=O)NC(Cc2ccccc2)C(=O)C(N)=O)n(-c2cccc(-c3ccccc3)c2)n1]

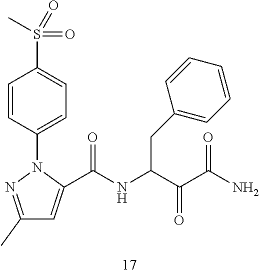 [SMILES: Cc1cc(C(=O)NC(Cc2ccccc2)C(=O)C(N)=O)n(-c2ccc(S(C)(=O)=O)cc2)n1]

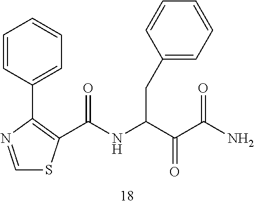 [SMILES: NC(=O)C(=O)C(Cc1ccccc1)NC(=O)c1scnc1-c1ccccc1]

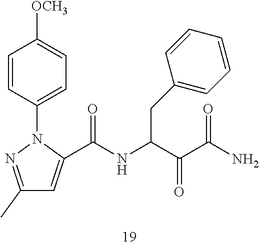 [SMILES: COc1ccc(-n2nc(C)cc2C(=O)NC(Cc2ccccc2)C(=O)C(N)=O)cc1]

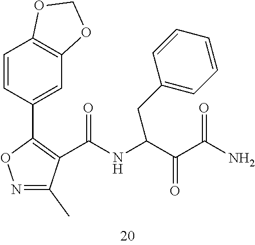 [SMILES: Cc1noc(-c2ccc3c(c2)OCO3)c1C(=O)NC(Cc1ccccc1)C(=O)C(N)=O]

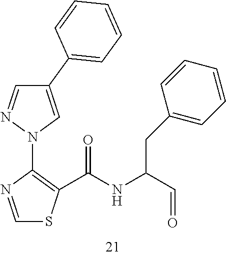 [SMILES: O=CC(Cc1ccccc1)NC(=O)c1scnc1-n1cc(-c2ccccc2)cn1]

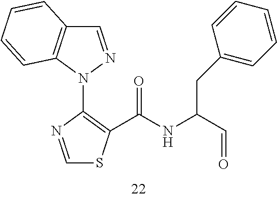 [SMILES: O=CC(Cc1ccccc1)NC(=O)c1scnc1-n1ncc2ccccc21]

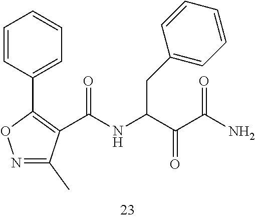 [SMILES: Cc1noc(-c2ccccc2)c1C(=O)NC(Cc1ccccc1)C(=O)C(N)=O]

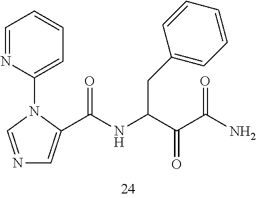 [SMILES: NC(=O)C(=O)C(Cc1ccccc1)NC(=O)c1cncn1-c1ccccn1]

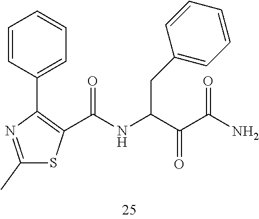 [SMILES: Cc1nc(-c2ccccc2)c(C(=O)NC(Cc2ccccc2)C(=O)C(N)=O)s1]

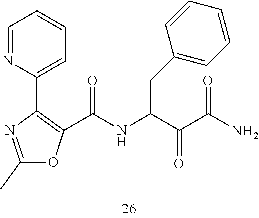 [SMILES: Cc1nc(-c2ccccn2)c(C(=O)NC(Cc2ccccc2)C(=O)C(N)=O)o1]

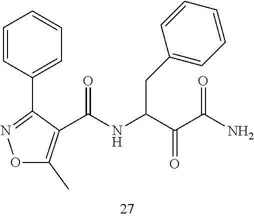 [SMILES: Cc1onc(-c2ccccc2)c1C(=O)NC(Cc1ccccc1)C(=O)C(N)=O]

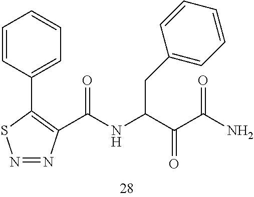 [SMILES: NC(=O)C(=O)C(Cc1ccccc1)NC(=O)c1nnsc1-c1ccccc1]

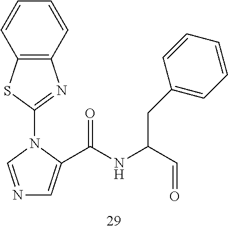 [SMILES: O=CC(Cc1ccccc1)NC(=O)c1cncn1-c1nc2ccccc2s1]

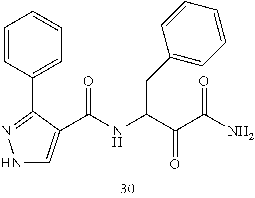 [SMILES: NC(=O)C(=O)C(Cc1ccccc1)NC(=O)c1c[nH]nc1-c1ccccc1]

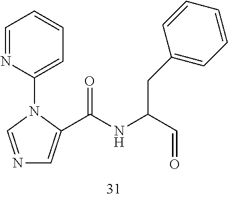 [SMILES: O=CC(Cc1ccccc1)NC(=O)c1cncn1-c1ccccn1]

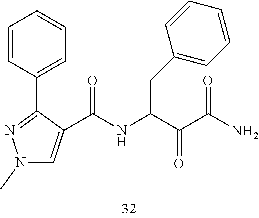 [SMILES: Cn1cc(C(=O)NC(Cc2ccccc2)C(=O)C(N)=O)c(-c2ccccc2)n1]

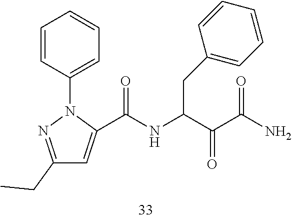 [SMILES: CCc1cc(C(=O)NC(Cc2ccccc2)C(=O)C(N)=O)n(-c2ccccc2)n1]

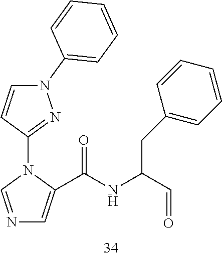 [SMILES: O=CC(Cc1ccccc1)NC(=O)c1cncn1-c1ccn(-c2ccccc2)n1]

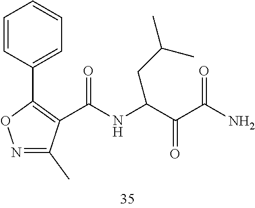 [SMILES: Cc1noc(-c2ccccc2)c1C(=O)NC(CC(C)C)C(=O)C(N)=O]

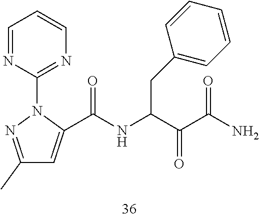 [SMILES: Cc1cc(C(=O)NC(Cc2ccccc2)C(=O)C(N)=O)n(-c2ncccn2)n1]

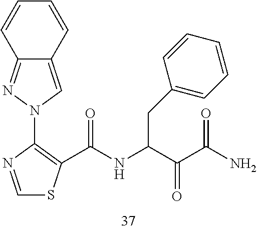 [SMILES: NC(=O)C(=O)C(Cc1ccccc1)NC(=O)c1scnc1-n1cc2ccccc2n1]

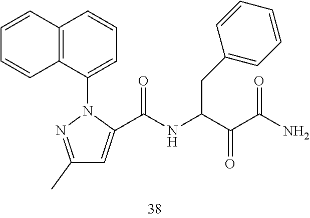 [SMILES: Cc1cc(C(=O)NC(Cc2ccccc2)C(=O)C(N)=O)n(-c2cccc3ccccc23)n1]

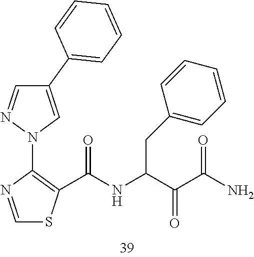 [SMILES: NC(=O)C(=O)C(Cc1ccccc1)NC(=O)c1scnc1-n1cc(-c2ccccc2)cn1]

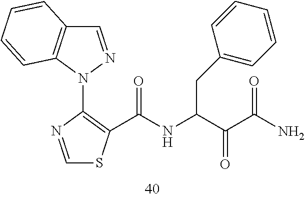 [SMILES: NC(=O)C(=O)C(Cc1ccccc1)NC(=O)c1scnc1-n1ncc2ccccc21]

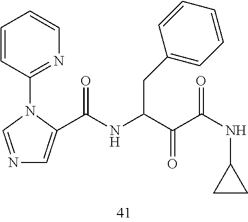 [SMILES: O=C(NC1CC1)C(=O)C(Cc1ccccc1)NC(=O)c1cncn1-c1ccccn1]

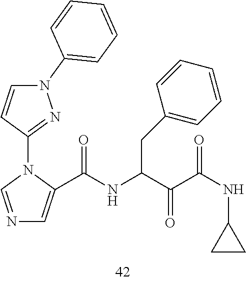 [SMILES: O=C(NC1CC1)C(=O)C(Cc1ccccc1)NC(=O)c1cncn1-c1ccn(-c2ccccc2)n1]

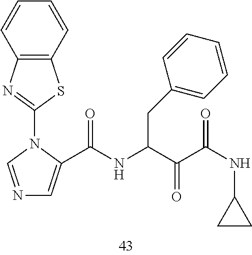 [SMILES: O=C(NC1CC1)C(=O)C(Cc1ccccc1)NC(=O)c1cncn1-c1nc2ccccc2s1]

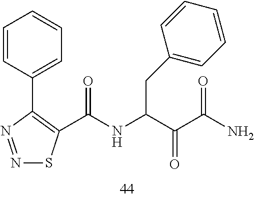 [SMILES: NC(=O)C(=O)C(Cc1ccccc1)NC(=O)c1snnc1-c1ccccc1]

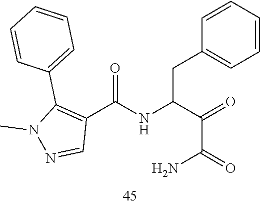 [SMILES: Cn1ncc(C(=O)NC(Cc2ccccc2)C(=O)C(N)=O)c1-c1ccccc1]

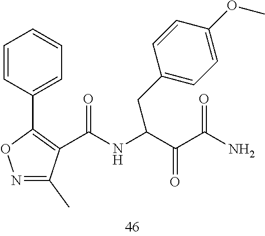 [SMILES: COc1ccc(CC(NC(=O)c2c(C)noc2-c2ccccc2)C(=O)C(N)=O)cc1]

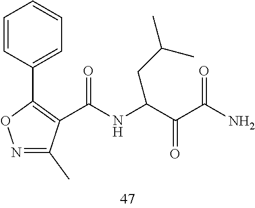 [SMILES: Cc1noc(-c2ccccc2)c1C(=O)NC(CC(C)C)C(=O)C(N)=O]

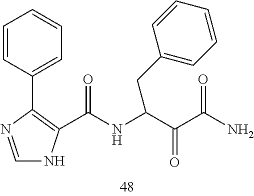 [SMILES: NC(=O)C(=O)C(Cc1ccccc1)NC(=O)c1[nH]cnc1-c1ccccc1]

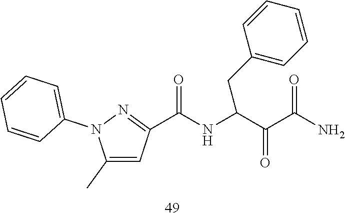

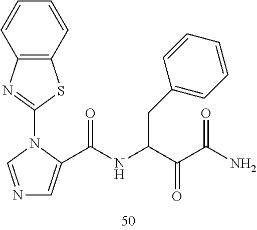 [SMILES: NC(=O)C(=O)C(Cc1ccccc1)NC(=O)c1cncn1-c1nc2ccccc2s1]

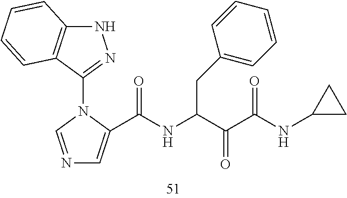 [SMILES: O=C(NC1CC1)C(=O)C(Cc1ccccc1)NC(=O)c1cncn1-c1n[nH]c2ccccc12]

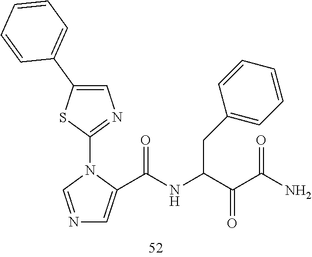 [SMILES: NC(=O)C(=O)C(Cc1ccccc1)NC(=O)c1cncn1-c1ncc(-c2ccccc2)s1]

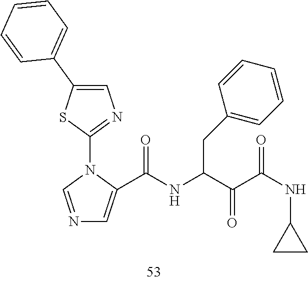 [SMILES: O=C(NC1CC1)C(=O)C(Cc1ccccc1)NC(=O)c1cncn1-c1ncc(-c2ccccc2)s1]

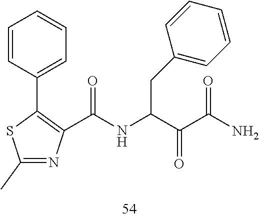 [SMILES: Cc1nc(C(=O)NC(Cc2ccccc2)C(=O)C(N)=O)c(-c2ccccc2)s1]

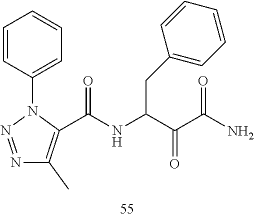 [SMILES: Cc1nnn(-c2ccccc2)c1C(=O)NC(Cc1ccccc1)C(=O)C(N)=O]

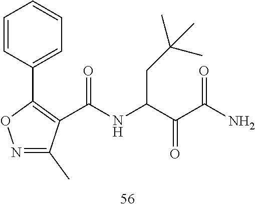 [SMILES: Cc1noc(-c2ccccc2)c1C(=O)NC(CC(C)(C)C)C(=O)C(N)=O]

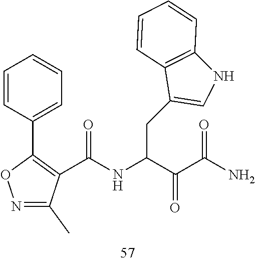 [SMILES: Cc1noc(-c2ccccc2)c1C(=O)NC(Cc1c[nH]c2ccccc12)C(=O)C(N)=O]

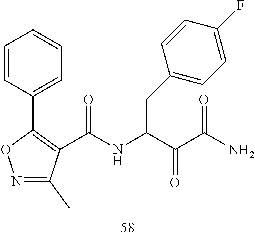 [SMILES: Cc1noc(-c2ccccc2)c1C(=O)NC(Cc1ccc(F)cc1)C(=O)C(N)=O]

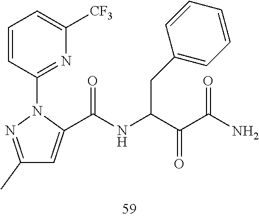 [SMILES: Cc1cc(C(=O)NC(Cc2ccccc2)C(=O)C(N)=O)n(-c2cccc(C(F)(F)F)n2)n1]

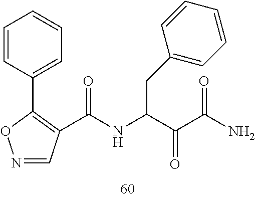 [SMILES: NC(=O)C(=O)C(Cc1ccccc1)NC(=O)c1cnoc1-c1ccccc1]

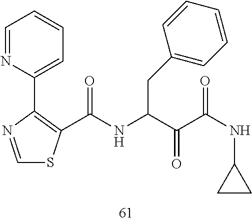 [SMILES: O=C(NC1CC1)C(=O)C(Cc1ccccc1)NC(=O)c1scnc1-c1ccccn1]

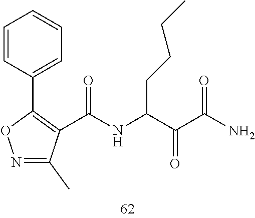 [SMILES: CCCCC(NC(=O)c1c(C)noc1-c1ccccc1)C(=O)C(N)=O]

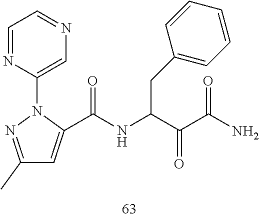 [SMILES: Cc1cc(C(=O)NC(Cc2ccccc2)C(=O)C(N)=O)n(-c2cnccn2)n1]

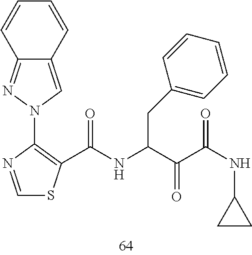 [SMILES: O=C(NC1CC1)C(=O)C(Cc1ccccc1)NC(=O)c1scnc1-n1cc2ccccc2n1]

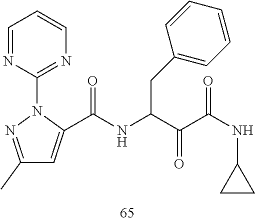 [SMILES: Cc1cc(C(=O)NC(Cc2ccccc2)C(=O)C(=O)NC2CC2)n(-c2ncccn2)n1]

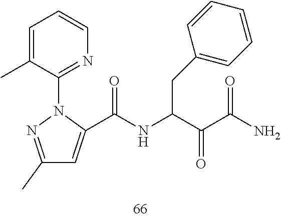 [SMILES: Cc1cc(C(=O)NC(Cc2ccccc2)C(=O)C(N)=O)n(-c2ncccc2C)n1]

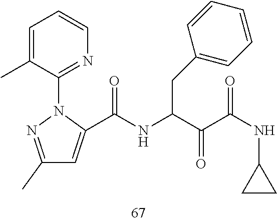 [SMILES: Cc1cc(C(=O)NC(Cc2ccccc2)C(=O)C(=O)NC2CC2)n(-c2ncccc2C)n1]

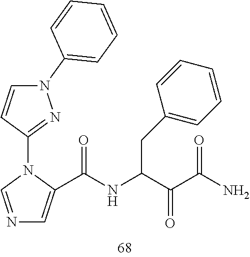 [SMILES: NC(=O)C(=O)C(Cc1ccccc1)NC(=O)c1cncn1-c1ccn(-c2ccccc2)n1]

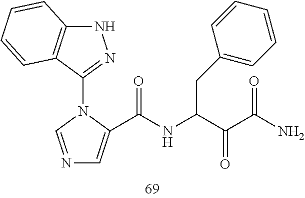 [SMILES: NC(=O)C(=O)C(Cc1ccccc1)NC(=O)c1cncn1-c1n[nH]c2ccccc12]

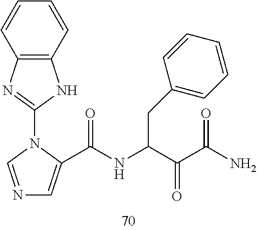 [SMILES: NC(=O)C(=O)C(Cc1ccccc1)NC(=O)c1cncn1-c1nc2ccccc2[nH]1]

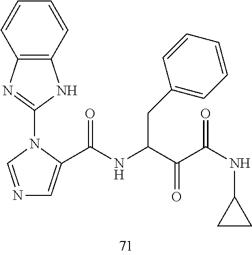 [SMILES: O=C(NC1CC1)C(=O)C(Cc1ccccc1)NC(=O)c1cncn1-c1nc2ccccc2[nH]1]

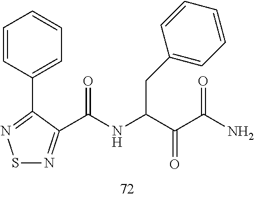 [SMILES: NC(=O)C(=O)C(Cc1ccccc1)NC(=O)c1nsnc1-c1ccccc1]

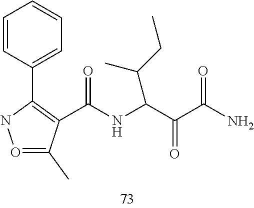

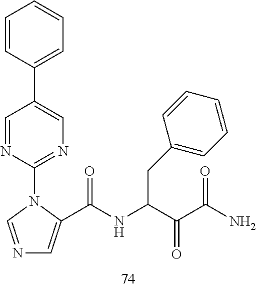 [SMILES: NC(=O)C(=O)C(Cc1ccccc1)NC(=O)c1cncn1-c1ncc(-c2ccccc2)cn1]

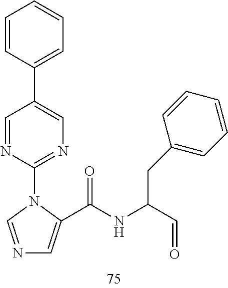 [SMILES: O=CC(Cc1ccccc1)NC(=O)c1cncn1-c1ncc(-c2ccccc2)cn1]

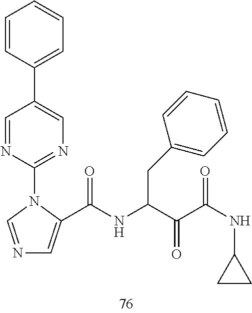 [SMILES: O=C(NC1CC1)C(=O)C(Cc1ccccc1)NC(=O)c1cncn1-c1ncc(-c2ccccc2)cn1]

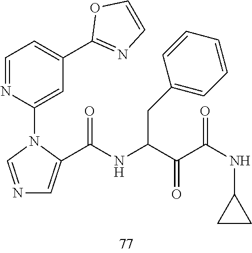 [SMILES: O=C(NC1CC1)C(=O)C(Cc1ccccc1)NC(=O)c1cncn1-c1cc(-c2ncco2)ccn1]

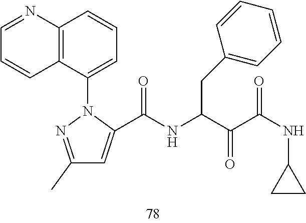 [SMILES: Cc1cc(C(=O)NC(Cc2ccccc2)C(=O)C(=O)NC2CC2)n(-c2cccc3ncccc23)n1]

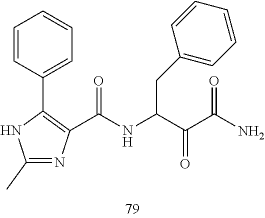 [SMILES: Cc1nc(C(=O)NC(Cc2ccccc2)C(=O)C(N)=O)c(-c2ccccc2)[nH]1]

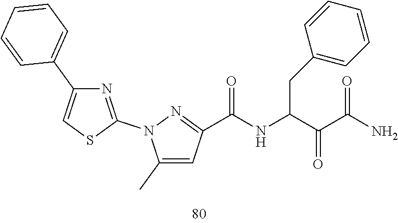 [SMILES: Cc1cc(C(=O)NC(Cc2ccccc2)C(=O)C(N)=O)nn1-c1nc(-c2ccccc2)cs1]

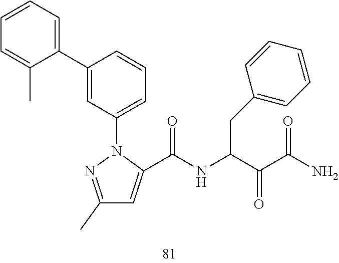 [SMILES: Cc1cc(C(=O)NC(Cc2ccccc2)C(=O)C(N)=O)n(-c2cccc(-c3ccccc3C)c2)n1]

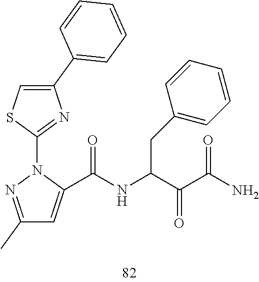 [SMILES: Cc1cc(C(=O)NC(Cc2ccccc2)C(=O)C(N)=O)n(-c2nc(-c3ccccc3)cs2)n1]

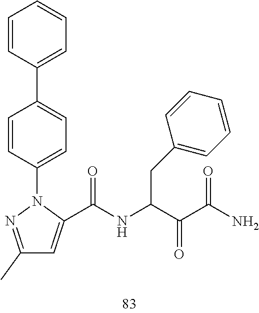 [SMILES: Cc1cc(C(=O)NC(Cc2ccccc2)C(=O)C(N)=O)n(-c2ccc(-c3ccccc3)cc2)n1]

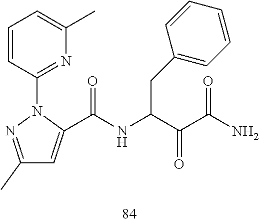 [SMILES: Cc1cccc(-n2nc(C)cc2C(=O)NC(Cc2ccccc2)C(=O)C(N)=O)n1]

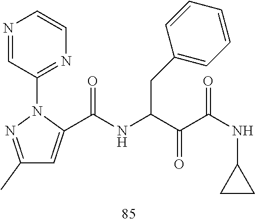 [SMILES: Cc1cc(C(=O)NC(Cc2ccccc2)C(=O)C(=O)NC2CC2)n(-c2cnccn2)n1]

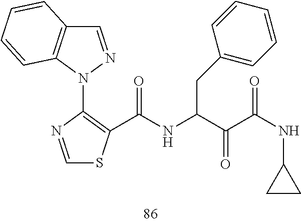 [SMILES: O=C(NC1CC1)C(=O)C(Cc1ccccc1)NC(=O)c1scnc1-n1ncc2ccccc21]

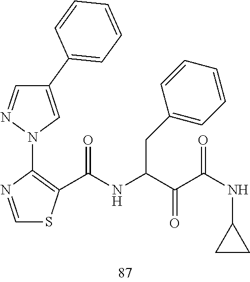 [SMILES: O=C(NC1CC1)C(=O)C(Cc1ccccc1)NC(=O)c1scnc1-n1cc(-c2ccccc2)cn1]

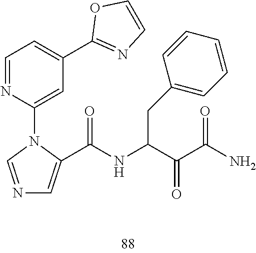 [SMILES: NC(=O)C(=O)C(Cc1ccccc1)NC(=O)c1cncn1-c1cc(-c2ncco2)ccn1]

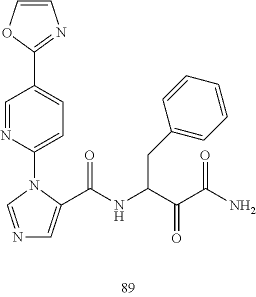 [SMILES: NC(=O)C(=O)C(Cc1ccccc1)NC(=O)c1cncn1-c1ccc(-c2ncco2)cn1]

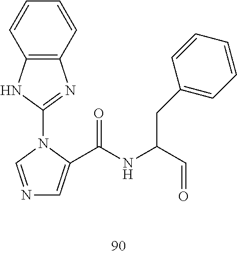 [SMILES: O=CC(Cc1ccccc1)NC(=O)c1cncn1-c1nc2ccccc2[nH]1]

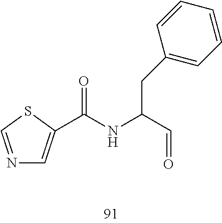 [SMILES: O=CC(Cc1ccccc1)NC(=O)c1cncs1]

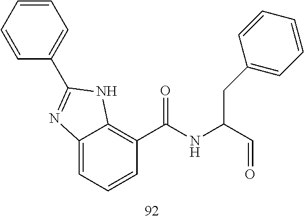 [SMILES: O=CC(Cc1ccccc1)NC(=O)c1cccc2nc(-c3ccccc3)[nH]c12]

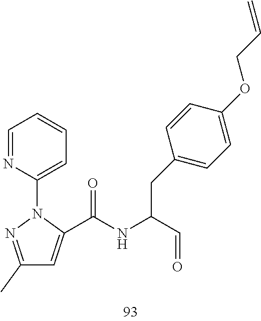 [SMILES: C=CCOc1ccc(CC(C=O)NC(=O)c2cc(C)nn2-c2ccccn2)cc1]

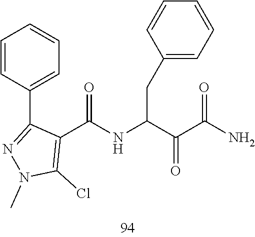 [SMILES: Cn1nc(-c2ccccc2)c(C(=O)NC(Cc2ccccc2)C(=O)C(N)=O)c1Cl]

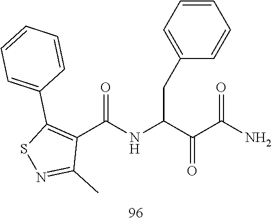 [SMILES: Cc1nsc(-c2ccccc2)c1C(=O)NC(Cc1ccccc1)C(=O)C(N)=O]

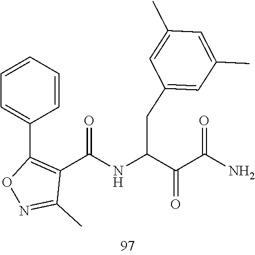 [SMILES: Cc1cc(C)cc(CC(NC(=O)c2c(C)noc2-c2ccccc2)C(=O)C(N)=O)c1]

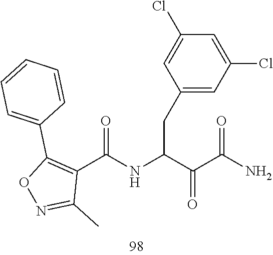 [SMILES: Cc1noc(-c2ccccc2)c1C(=O)NC(Cc1cc(Cl)cc(Cl)c1)C(=O)C(N)=O]

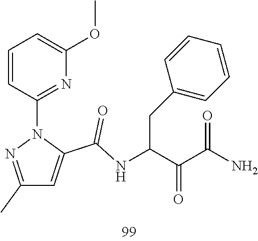 [SMILES: COc1cccc(-n2nc(C)cc2C(=O)NC(Cc2ccccc2)C(=O)C(N)=O)n1]

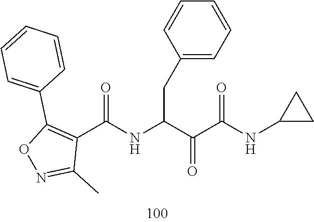 [SMILES: Cc1noc(-c2ccccc2)c1C(=O)NC(Cc1ccccc1)C(=O)C(=O)NC1CC1]

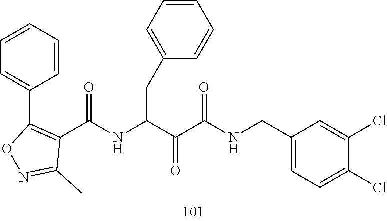 [SMILES: Cc1noc(-c2ccccc2)c1C(=O)NC(Cc1ccccc1)C(=O)C(=O)NCc1ccc(Cl)c(Cl)c1]

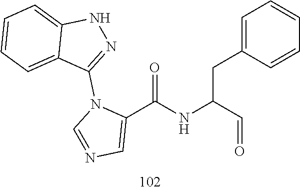 [SMILES: O=CC(Cc1ccccc1)NC(=O)c1cncn1-c1n[nH]c2ccccc12]

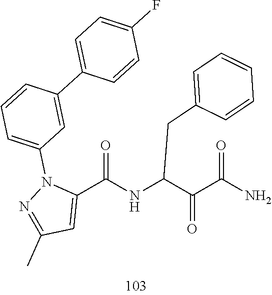 [SMILES: Cc1cc(C(=O)NC(Cc2ccccc2)C(=O)C(N)=O)n(-c2cccc(-c3ccc(F)cc3)c2)n1]

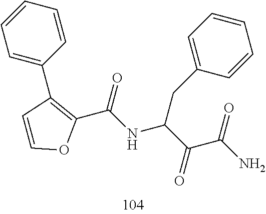 [SMILES: NC(=O)C(=O)C(Cc1ccccc1)NC(=O)c1occc1-c1ccccc1]

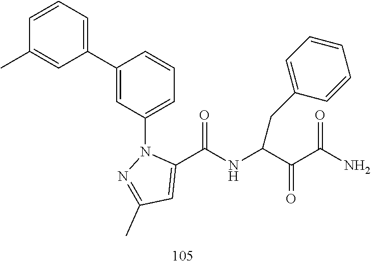 [SMILES: Cc1cccc(-c2cccc(-n3nc(C)cc3C(=O)NC(Cc3ccccc3)C(=O)C(N)=O)c2)c1]

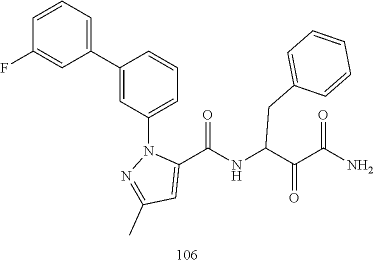 [SMILES: Cc1cc(C(=O)NC(Cc2ccccc2)C(=O)C(N)=O)n(-c2cccc(-c3cccc(F)c3)c2)n1]

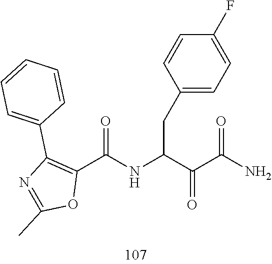 [SMILES: Cc1nc(-c2ccccc2)c(C(=O)NC(Cc2ccc(F)cc2)C(=O)C(N)=O)o1]

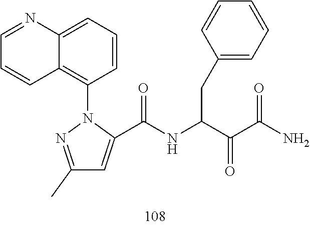 [SMILES: Cc1cc(C(=O)NC(Cc2ccccc2)C(=O)C(N)=O)n(-c2cccc3ncccc23)n1]

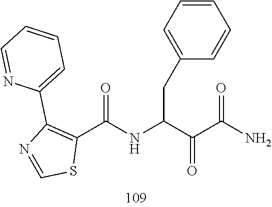 [SMILES: NC(=O)C(=O)C(Cc1ccccc1)NC(=O)c1scnc1-c1ccccn1]

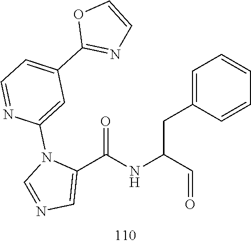 [SMILES: O=CC(Cc1ccccc1)NC(=O)c1cncn1-c1cc(-c2ncco2)ccn1]

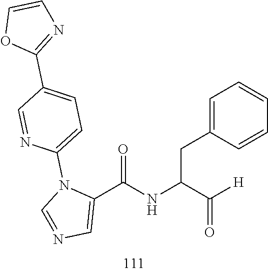 [SMILES: O=CC(Cc1ccccc1)NC(=O)c1cncn1-c1ccc(-c2ncco2)cn1]

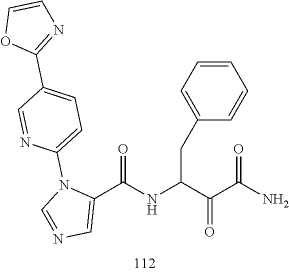 [SMILES: NC(=O)C(=O)C(Cc1ccccc1)NC(=O)c1cncn1-c1ccc(-c2ncco2)cn1]

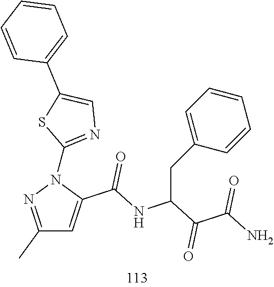 [SMILES: Cc1cc(C(=O)NC(Cc2ccccc2)C(=O)C(N)=O)n(-c2ncc(-c3ccccc3)s2)n1]

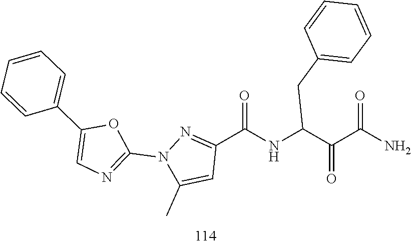 [SMILES: Cc1cc(C(=O)NC(Cc2ccccc2)C(=O)C(N)=O)nn1-c1ncc(-c2ccccc2)o1]

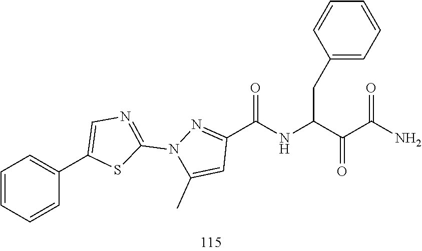 [SMILES: Cc1cc(C(=O)NC(Cc2ccccc2)C(=O)C(N)=O)nn1-c1ncc(-c2ccccc2)s1]

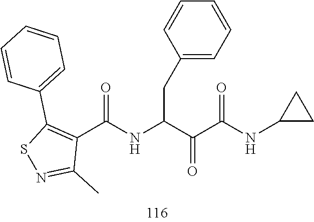 [SMILES: Cc1nsc(-c2ccccc2)c1C(=O)NC(Cc1ccccc1)C(=O)C(=O)NC1CC1]

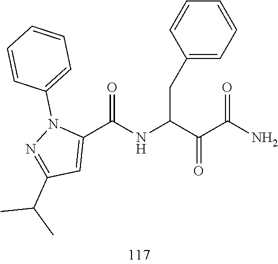 [SMILES: CC(C)c1cc(C(=O)NC(Cc2ccccc2)C(=O)C(N)=O)n(-c2ccccc2)n1]

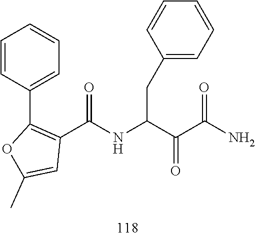 [SMILES: Cc1cc(C(=O)NC(Cc2ccccc2)C(=O)C(N)=O)c(-c2ccccc2)o1]

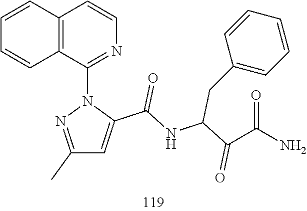 [SMILES: Cc1cc(C(=O)NC(Cc2ccccc2)C(=O)C(N)=O)n(-c2nccc3ccccc23)n1]

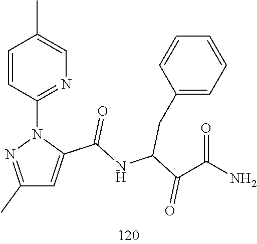 [SMILES: Cc1ccc(-n2nc(C)cc2C(=O)NC(Cc2ccccc2)C(=O)C(N)=O)nc1]

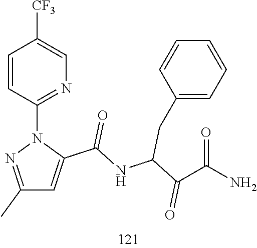 [SMILES: Cc1cc(C(=O)NC(Cc2ccccc2)C(=O)C(N)=O)n(-c2ccc(C(F)(F)F)cn2)n1]

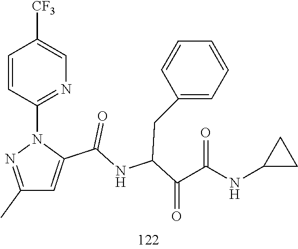 [SMILES: Cc1cc(C(=O)NC(Cc2ccccc2)C(=O)C(=O)NC2CC2)n(-c2ccc(C(F)(F)F)cn2)n1]

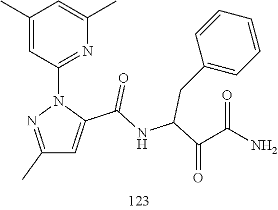 [SMILES: Cc1cc(C)nc(-n2nc(C)cc2C(=O)NC(Cc2ccccc2)C(=O)C(N)=O)c1]

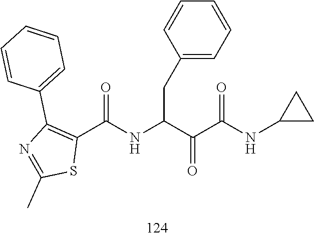 [SMILES: Cc1nc(-c2ccccc2)c(C(=O)NC(Cc2ccccc2)C(=O)C(=O)NC2CC2)s1]

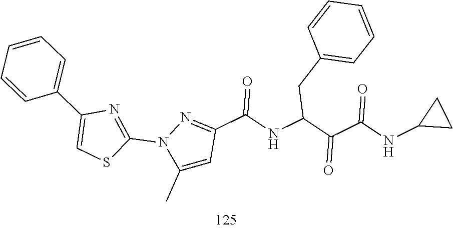 [SMILES: Cc1cc(C(=O)NC(Cc2ccccc2)C(=O)C(=O)NC2CC2)nn1-c1nc(-c2ccccc2)cs1]

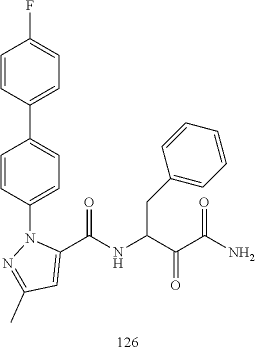 [SMILES: Cc1cc(C(=O)NC(Cc2ccccc2)C(=O)C(N)=O)n(-c2ccc(-c3ccc(F)cc3)cc2)n1]

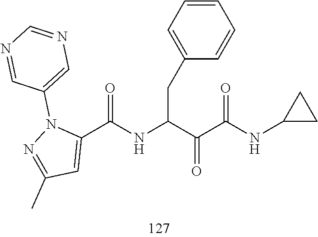 [SMILES: Cc1cc(C(=O)NC(Cc2ccccc2)C(=O)C(=O)NC2CC2)n(-c2cncnc2)n1]

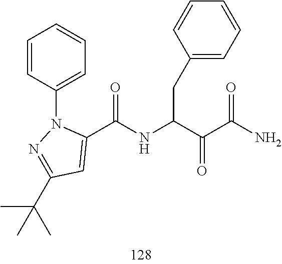 [SMILES: CC(C)(C)c1cc(C(=O)NC(Cc2ccccc2)C(=O)C(N)=O)n(-c2ccccc2)n1]

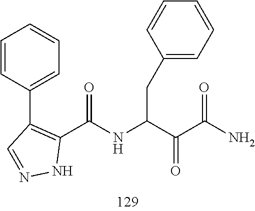 [SMILES: NC(=O)C(=O)C(Cc1ccccc1)NC(=O)c1[nH]ncc1-c1ccccc1]

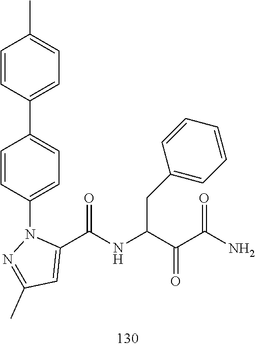 [SMILES: Cc1ccc(-c2ccc(-n3nc(C)cc3C(=O)NC(Cc3ccccc3)C(=O)C(N)=O)cc2)cc1]

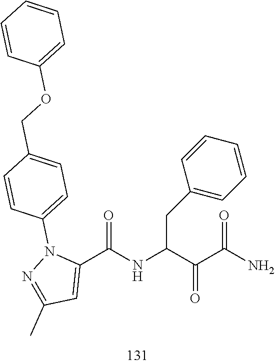 [SMILES: Cc1cc(C(=O)NC(Cc2ccccc2)C(=O)C(N)=O)n(-c2ccc(COc3ccccc3)cc2)n1]

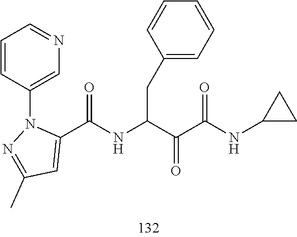 [SMILES: Cc1cc(C(=O)NC(Cc2ccccc2)C(=O)C(=O)NC2CC2)n(-c2cccnc2)n1]

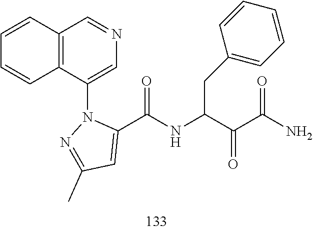 [SMILES: Cc1cc(C(=O)NC(Cc2ccccc2)C(=O)C(N)=O)n(-c2cncc3ccccc23)n1]

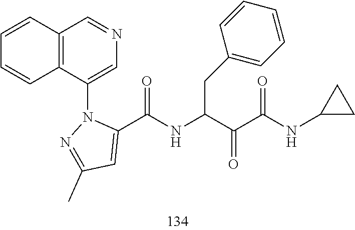 [SMILES: Cc1cc(C(=O)NC(Cc2ccccc2)C(=O)C(=O)NC2CC2)n(-c2cncc3ccccc23)n1]

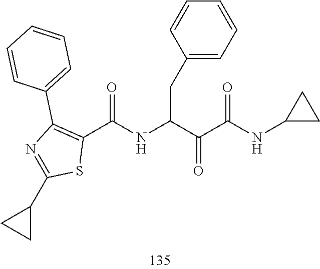 [SMILES: O=C(NC1CC1)C(=O)C(Cc1ccccc1)NC(=O)c1sc(C2CC2)nc1-c1ccccc1]

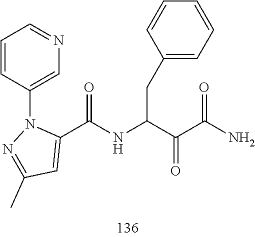 [SMILES: Cc1cc(C(=O)NC(Cc2ccccc2)C(=O)C(N)=O)n(-c2cccnc2)n1]

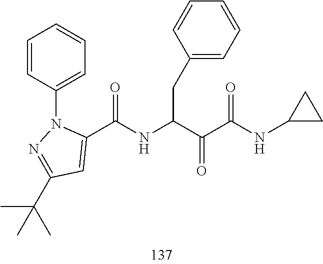 [SMILES: CC(C)(C)c1cc(C(=O)NC(Cc2ccccc2)C(=O)C(=O)NC2CC2)n(-c2ccccc2)n1]

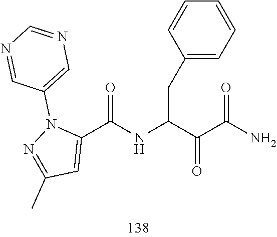 [SMILES: Cc1cc(C(=O)NC(Cc2ccccc2)C(=O)C(N)=O)n(-c2cncnc2)n1]

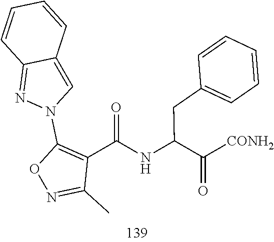 [SMILES: Cc1noc(-n2cc3ccccc3n2)c1C(=O)NC(Cc1ccccc1)C(=O)C(N)=O]

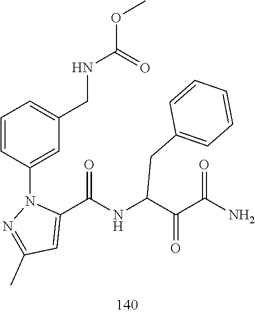 [SMILES: COC(=O)NCc1cccc(-n2nc(C)cc2C(=O)NC(Cc2ccccc2)C(=O)C(N)=O)c1]

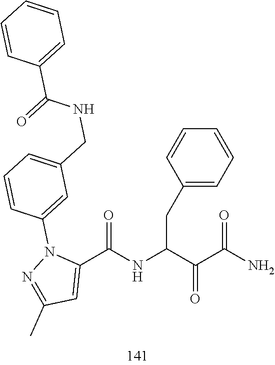 [SMILES: Cc1cc(C(=O)NC(Cc2ccccc2)C(=O)C(N)=O)n(-c2cccc(CNC(=O)c3ccccc3)c2)n1]

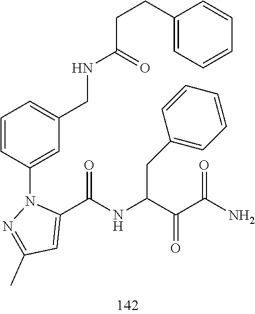 [SMILES: Cc1cc(C(=O)NC(Cc2ccccc2)C(=O)C(N)=O)n(-c2cccc(CNC(=O)CCc3ccccc3)c2)n1]

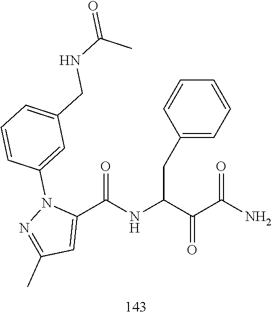 [SMILES: CC(=O)NCc1cccc(-n2nc(C)cc2C(=O)NC(Cc2ccccc2)C(=O)C(N)=O)c1]

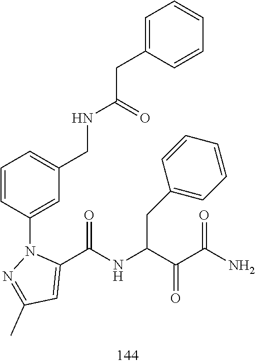 [SMILES: Cc1cc(C(=O)NC(Cc2ccccc2)C(=O)C(N)=O)n(-c2cccc(CNC(=O)Cc3ccccc3)c2)n1]

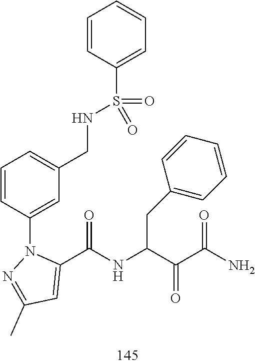 [SMILES: Cc1cc(C(=O)NC(Cc2ccccc2)C(=O)C(N)=O)n(-c2cccc(CNS(=O)(=O)c3ccccc3)c2)n1]

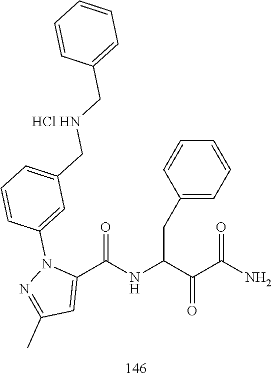

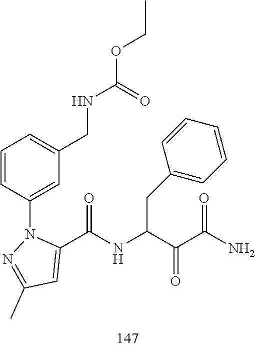 [SMILES: CCOC(=O)NCc1cccc(-n2nc(C)cc2C(=O)NC(Cc2ccccc2)C(=O)C(N)=O)c1]

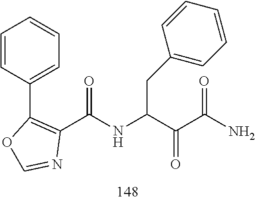 [SMILES: NC(=O)C(=O)C(Cc1ccccc1)NC(=O)c1ncoc1-c1ccccc1]

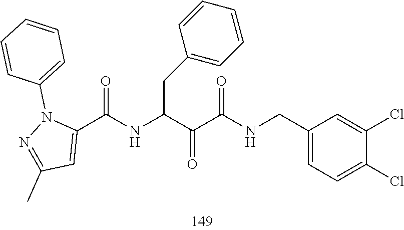 [SMILES: Cc1cc(C(=O)NC(Cc2ccccc2)C(=O)C(=O)NCc2ccc(Cl)c(Cl)c2)n(-c2ccccc2)n1]

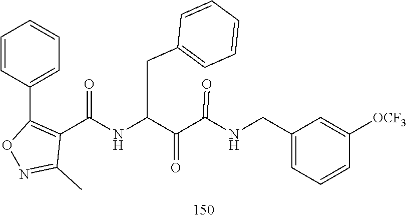 [SMILES: Cc1noc(-c2ccccc2)c1C(=O)NC(Cc1ccccc1)C(=O)C(=O)NCc1cccc(OC(F)(F)F)c1]

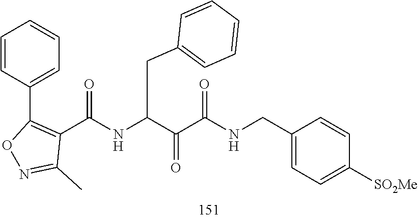 [SMILES: Cc1noc(-c2ccccc2)c1C(=O)NC(Cc1ccccc1)C(=O)C(=O)NCc1ccc(S(C)(=O)=O)cc1]

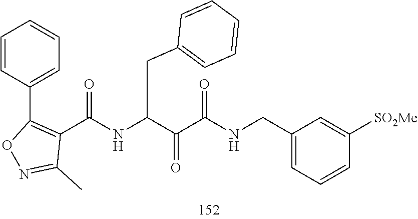 [SMILES: Cc1noc(-c2ccccc2)c1C(=O)NC(Cc1ccccc1)C(=O)C(=O)NCc1cccc(S(C)(=O)=O)c1]

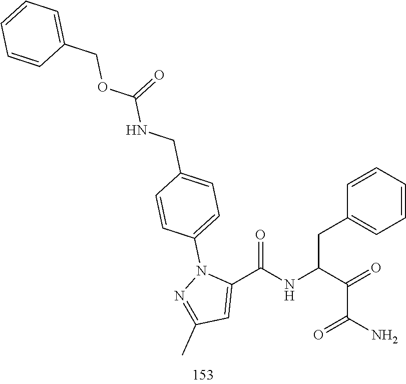 [SMILES: Cc1cc(C(=O)NC(Cc2ccccc2)C(=O)C(N)=O)n(-c2ccc(CNC(=O)OCc3ccccc3)cc2)n1]

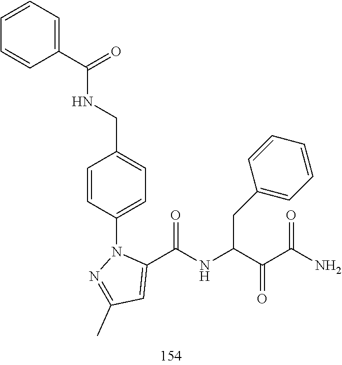 [SMILES: Cc1cc(C(=O)NC(Cc2ccccc2)C(=O)C(N)=O)n(-c2ccc(CNC(=O)c3ccccc3)cc2)n1]

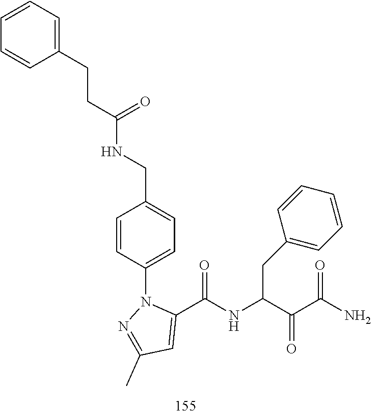 [SMILES: Cc1cc(C(=O)NC(Cc2ccccc2)C(=O)C(N)=O)n(-c2ccc(CNC(=O)CCc3ccccc3)cc2)n1]

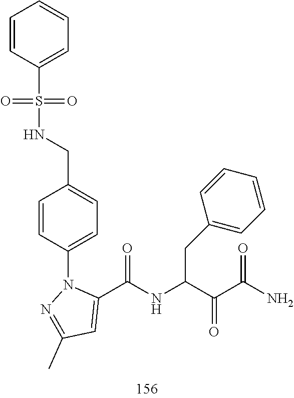 [SMILES: Cc1cc(C(=O)NC(Cc2ccccc2)C(=O)C(N)=O)n(-c2ccc(CNS(=O)(=O)c3ccccc3)cc2)n1]

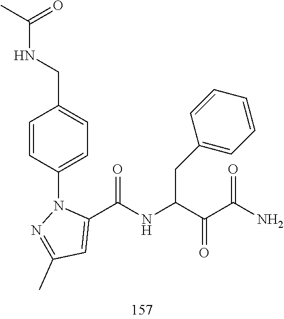 [SMILES: CC(=O)NCc1ccc(-n2nc(C)cc2C(=O)NC(Cc2ccccc2)C(=O)C(N)=O)cc1]

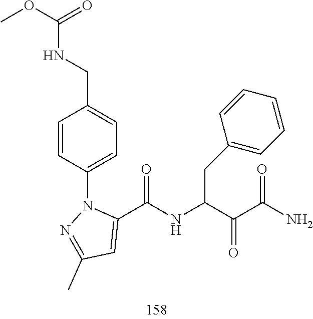 [SMILES: COC(=O)NCc1ccc(-n2nc(C)cc2C(=O)NC(Cc2ccccc2)C(=O)C(N)=O)cc1]

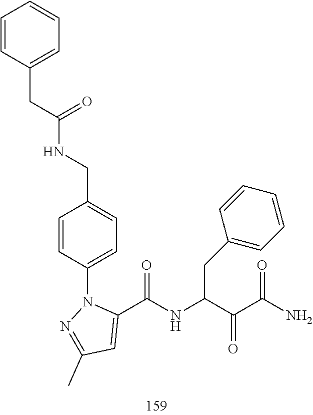 [SMILES: Cc1cc(C(=O)NC(Cc2ccccc2)C(=O)C(N)=O)n(-c2ccc(CNC(=O)Cc3ccccc3)cc2)n1]

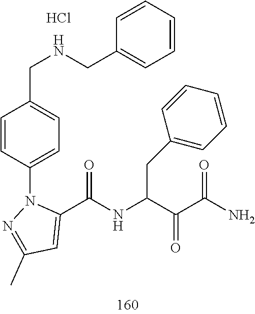 [SMILES: Cc1cc(C(=O)NC(Cc2ccccc2)C(=O)C(N)=O)n(-c2ccc(CNCc3ccccc3)cc2)n1 ; Cl]

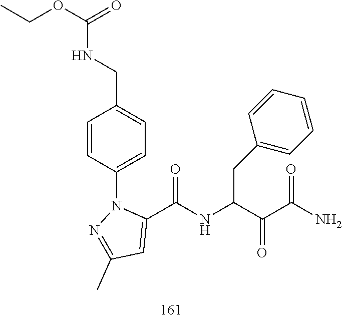 [SMILES: CCOC(=O)NCc1ccc(-n2nc(C)cc2C(=O)NC(Cc2ccccc2)C(=O)C(N)=O)cc1]

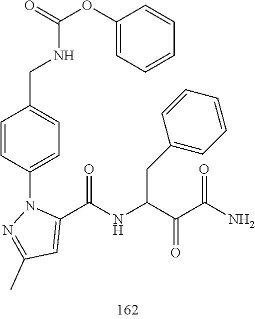 [SMILES: Cc1cc(C(=O)NC(Cc2ccccc2)C(=O)C(N)=O)n(-c2ccc(CNC(=O)Oc3ccccc3)cc2)n1]

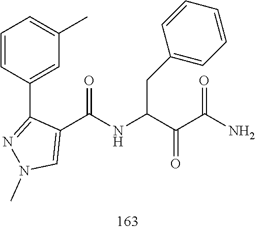 [SMILES: Cc1cccc(-c2nn(C)cc2C(=O)NC(Cc2ccccc2)C(=O)C(N)=O)c1]

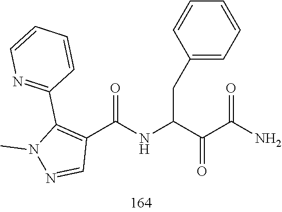 [SMILES: Cn1ncc(C(=O)NC(Cc2ccccc2)C(=O)C(N)=O)c1-c1ccccn1]

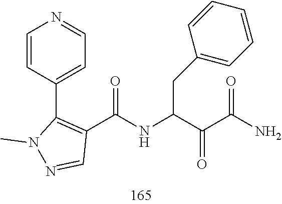 [SMILES: Cn1ncc(C(=O)NC(Cc2ccccc2)C(=O)C(N)=O)c1-c1ccncc1]

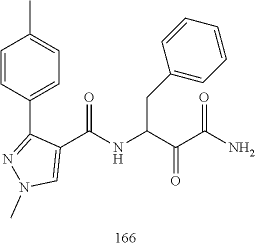 [SMILES: Cc1ccc(-c2nn(C)cc2C(=O)NC(Cc2ccccc2)C(=O)C(N)=O)cc1]

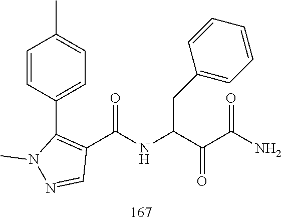 [SMILES: Cc1ccc(-c2c(C(=O)NC(Cc3ccccc3)C(=O)C(N)=O)cnn2C)cc1]

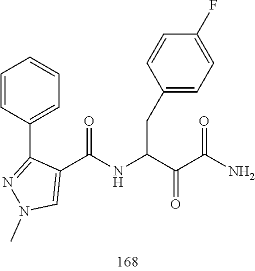 [SMILES: Cn1cc(C(=O)NC(Cc2ccc(F)cc2)C(=O)C(N)=O)c(-c2ccccc2)n1]

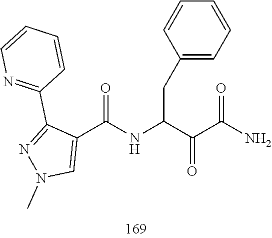 [SMILES: Cn1cc(C(=O)NC(Cc2ccccc2)C(=O)C(N)=O)c(-c2ccccn2)n1]

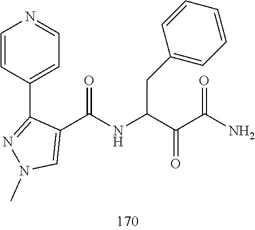 [SMILES: Cn1cc(C(=O)NC(Cc2ccccc2)C(=O)C(N)=O)c(-c2ccncc2)n1]

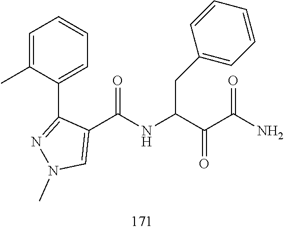 [SMILES: Cc1ccccc1-c1nn(C)cc1C(=O)NC(Cc1ccccc1)C(=O)C(N)=O]

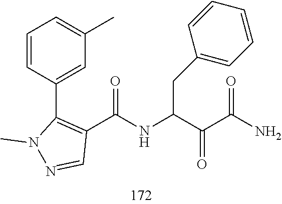 [SMILES: Cc1cccc(-c2c(C(=O)NC(Cc3ccccc3)C(=O)C(N)=O)cnn2C)c1]

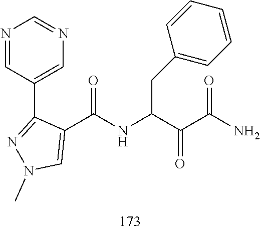 [SMILES: Cn1cc(C(=O)NC(Cc2ccccc2)C(=O)C(N)=O)c(-c2cncnc2)n1]

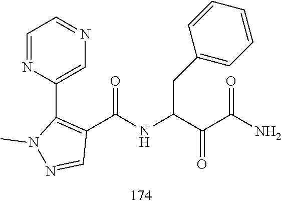 [SMILES: Cn1ncc(C(=O)NC(Cc2ccccc2)C(=O)C(N)=O)c1-c1cnccn1]

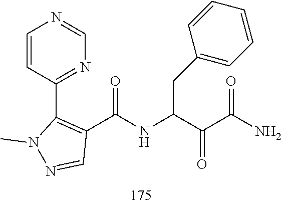 [SMILES: Cn1ncc(C(=O)NC(Cc2ccccc2)C(=O)C(N)=O)c1-c1ccncn1]

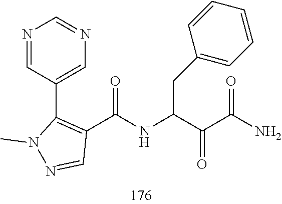 [SMILES: Cn1ncc(C(=O)NC(Cc2ccccc2)C(=O)C(N)=O)c1-c1cncnc1]

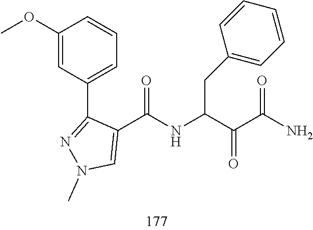 [SMILES: COc1cccc(-c2nn(C)cc2C(=O)NC(Cc2ccccc2)C(=O)C(N)=O)c1]

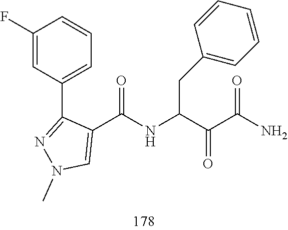 [SMILES: Cn1cc(C(=O)NC(Cc2ccccc2)C(=O)C(N)=O)c(-c2cccc(F)c2)n1]

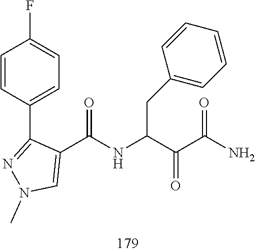 [SMILES: Cn1cc(C(=O)NC(Cc2ccccc2)C(=O)C(N)=O)c(-c2ccc(F)cc2)n1]

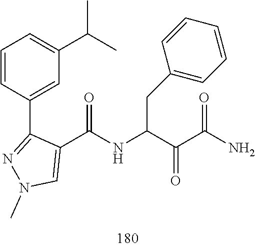 [SMILES: CC(C)c1cccc(-c2nn(C)cc2C(=O)NC(Cc2ccccc2)C(=O)C(N)=O)c1]

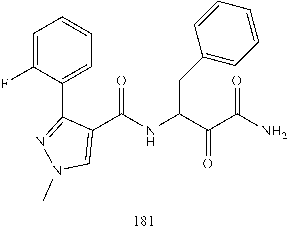 [SMILES: Cn1cc(C(=O)NC(Cc2ccccc2)C(=O)C(N)=O)c(-c2ccccc2F)n1]

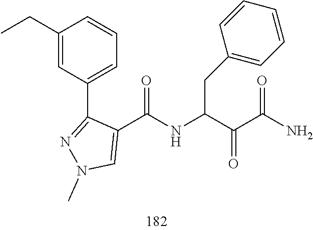 [SMILES: CCc1cccc(-c2nn(C)cc2C(=O)NC(Cc2ccccc2)C(=O)C(N)=O)c1]

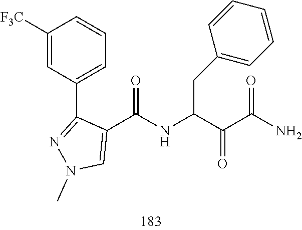 [SMILES: Cn1cc(C(=O)NC(Cc2ccccc2)C(=O)C(N)=O)c(-c2cccc(C(F)(F)F)c2)n1]

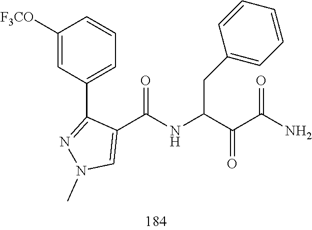 [SMILES: Cn1cc(C(=O)NC(Cc2ccccc2)C(=O)C(N)=O)c(-c2cccc(OC(F)(F)F)c2)n1]

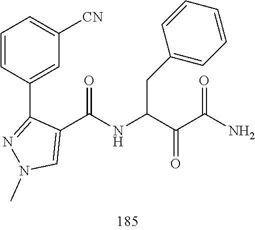 [SMILES: Cn1cc(C(=O)NC(Cc2ccccc2)C(=O)C(N)=O)c(-c2cccc(C#N)c2)n1]

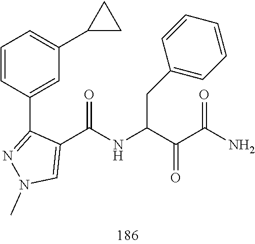 [SMILES: Cn1cc(C(=O)NC(Cc2ccccc2)C(=O)C(N)=O)c(-c2cccc(C3CC3)c2)n1]

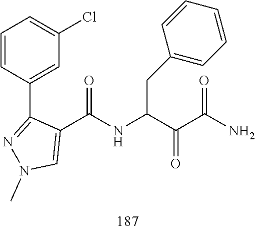 [SMILES: Cn1cc(C(=O)NC(Cc2ccccc2)C(=O)C(N)=O)c(-c2cccc(Cl)c2)n1]

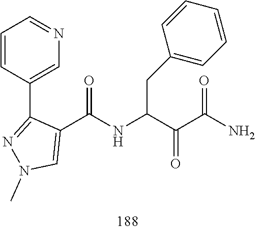 [SMILES: Cn1cc(C(=O)NC(Cc2ccccc2)C(=O)C(N)=O)c(-c2cccnc2)n1]

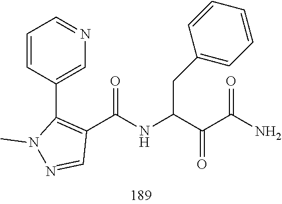 [SMILES: Cn1ncc(C(=O)NC(Cc2ccccc2)C(=O)C(N)=O)c1-c1cccnc1]

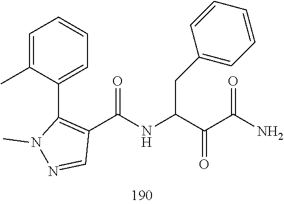 [SMILES: Cc1ccccc1-c1c(C(=O)NC(Cc2ccccc2)C(=O)C(N)=O)cnn1C]

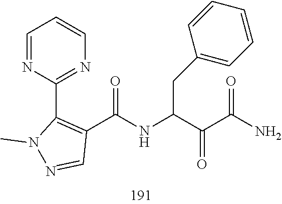 [SMILES: Cn1ncc(C(=O)NC(Cc2ccccc2)C(=O)C(N)=O)c1-c1ncccn1]

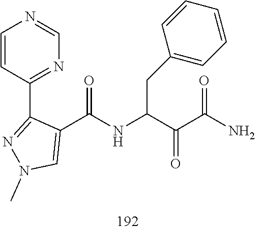 [SMILES: Cn1cc(C(=O)NC(Cc2ccccc2)C(=O)C(N)=O)c(-c2ccncn2)n1]

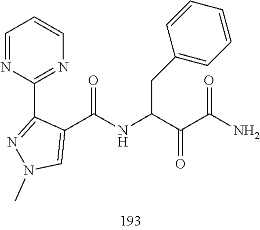 [SMILES: Cn1cc(C(=O)NC(Cc2ccccc2)C(=O)C(N)=O)c(-c2ncccn2)n1]

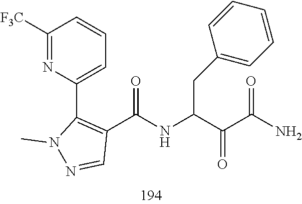 [SMILES: Cn1ncc(C(=O)NC(Cc2ccccc2)C(=O)C(N)=O)c1-c1cccc(C(F)(F)F)n1]

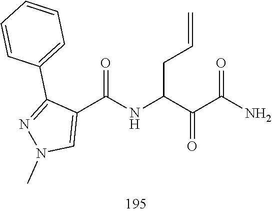 [SMILES: C=CCC(NC(=O)c1cn(C)nc1-c1ccccc1)C(=O)C(N)=O]

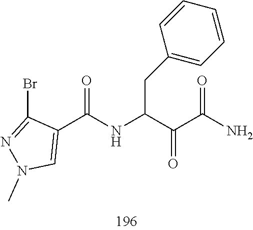 [SMILES: Cn1cc(C(=O)NC(Cc2ccccc2)C(=O)C(N)=O)c(Br)n1]

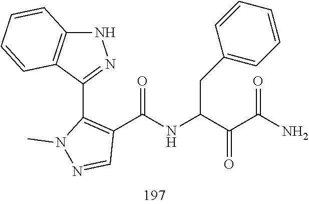 [SMILES: Cn1ncc(C(=O)NC(Cc2ccccc2)C(=O)C(N)=O)c1-c1n[nH]c2ccccc12]

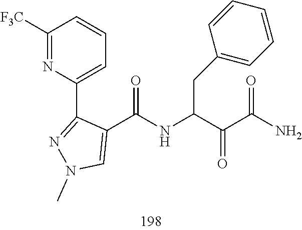 [SMILES: Cn1cc(C(=O)NC(Cc2ccccc2)C(=O)C(N)=O)c(-c2cccc(C(F)(F)F)n2)n1]

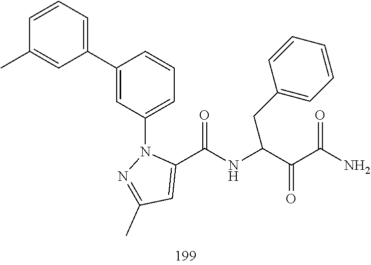

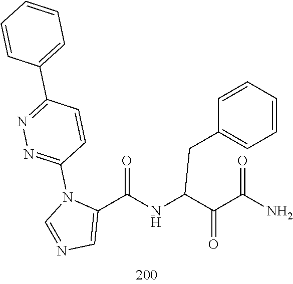 [SMILES: NC(=O)C(=O)C(Cc1ccccc1)NC(=O)c1cncn1-c1ccc(-c2ccccc2)nn1]

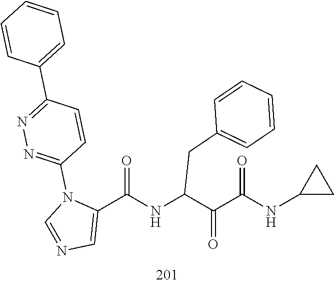 [SMILES: O=C(NC1CC1)C(=O)C(Cc1ccccc1)NC(=O)c1cncn1-c1ccc(-c2ccccc2)nn1]

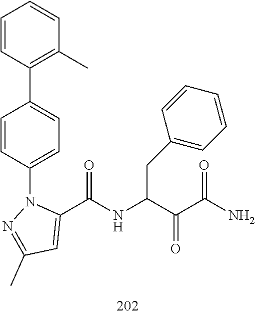 [SMILES: Cc1cc(C(=O)NC(Cc2ccccc2)C(=O)C(N)=O)n(-c2ccc(-c3ccccc3C)cc2)n1]

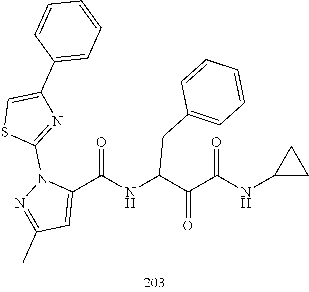 [SMILES: Cc1cc(C(=O)NC(Cc2ccccc2)C(=O)C(=O)NC2CC2)n(-c2nc(-c3ccccc3)cs2)n1]

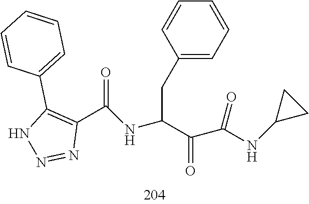 [SMILES: O=C(NC1CC1)C(=O)C(Cc1ccccc1)NC(=O)c1nn[nH]c1-c1ccccc1]

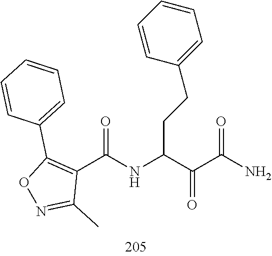 [SMILES: Cc1noc(-c2ccccc2)c1C(=O)NC(CCc1ccccc1)C(=O)C(N)=O]

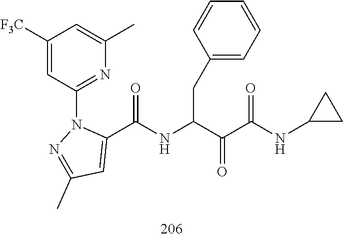 [SMILES: Cc1cc(C(F)(F)F)cc(-n2nc(C)cc2C(=O)NC(Cc2ccccc2)C(=O)C(=O)NC2CC2)n1]

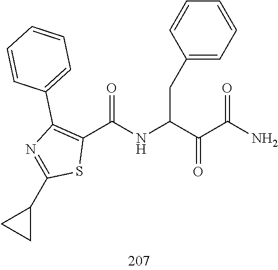 [SMILES: NC(=O)C(=O)C(Cc1ccccc1)NC(=O)c1sc(C2CC2)nc1-c1ccccc1]

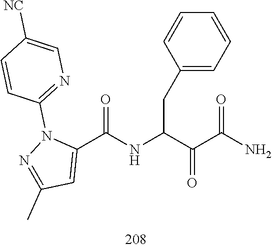 [SMILES: Cc1cc(C(=O)NC(Cc2ccccc2)C(=O)C(N)=O)n(-c2ccc(C#N)cn2)n1]

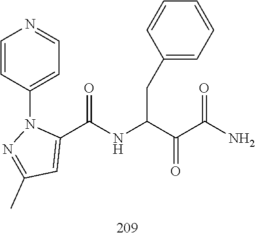 [SMILES: Cc1cc(C(=O)NC(Cc2ccccc2)C(=O)C(N)=O)n(-c2ccncc2)n1]

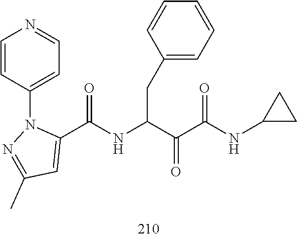 [SMILES: Cc1cc(C(=O)NC(Cc2ccccc2)C(=O)C(=O)NC2CC2)n(-c2ccncc2)n1]

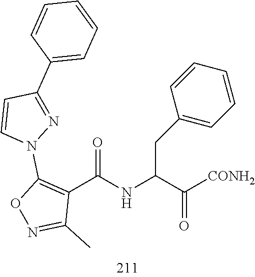 [SMILES: Cc1noc(-n2ccc(-c3ccccc3)n2)c1C(=O)NC(Cc1ccccc1)C(=O)C(N)=O]

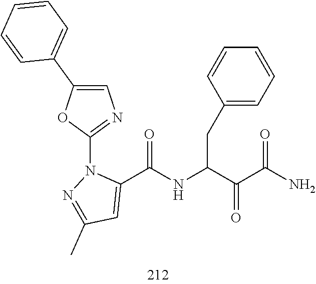 [SMILES: Cc1cc(C(=O)NC(Cc2ccccc2)C(=O)C(N)=O)n(-c2ncc(-c3ccccc3)o2)n1]

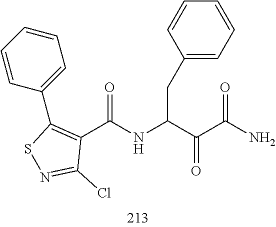 [SMILES: NC(=O)C(=O)C(Cc1ccccc1)NC(=O)c1c(Cl)nsc1-c1ccccc1]

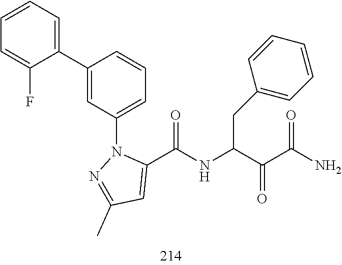 [SMILES: Cc1cc(C(=O)NC(Cc2ccccc2)C(=O)C(N)=O)n(-c2cccc(-c3ccccc3F)c2)n1]

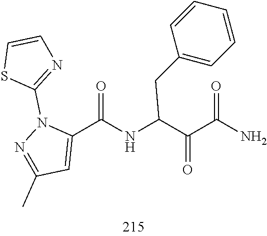 [SMILES: Cc1cc(C(=O)NC(Cc2ccccc2)C(=O)C(N)=O)n(-c2nccs2)n1]

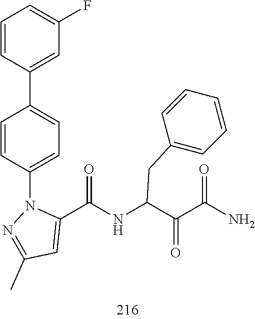 [SMILES: Cc1cc(C(=O)NC(Cc2ccccc2)C(=O)C(N)=O)n(-c2ccc(-c3cccc(F)c3)cc2)n1]

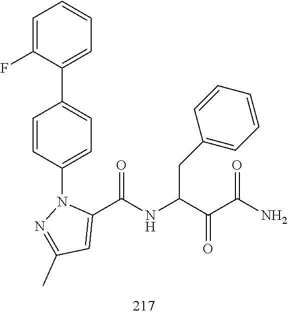 [SMILES: Cc1cc(C(=O)NC(Cc2ccccc2)C(=O)C(N)=O)n(-c2ccc(-c3ccccc3F)cc2)n1]

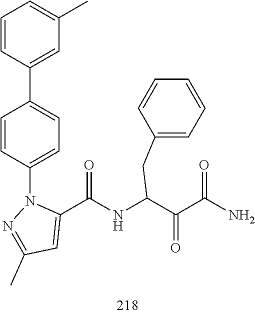 [SMILES: Cc1cccc(-c2ccc(-n3nc(C)cc3C(=O)NC(Cc3ccccc3)C(=O)C(N)=O)cc2)c1]

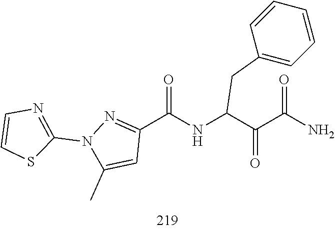 [SMILES: Cc1cc(C(=O)NC(Cc2ccccc2)C(=O)C(N)=O)nn1-c1nccs1]

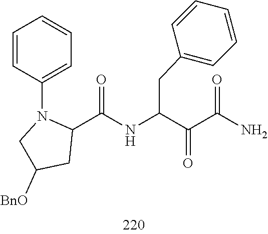 [SMILES: NC(=O)C(=O)C(Cc1ccccc1)NC(=O)C1CC(OCc2ccccc2)CN1c1ccccc1]

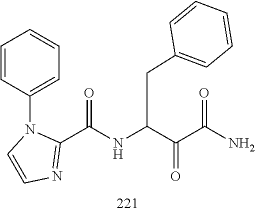 [SMILES: NC(=O)C(=O)C(Cc1ccccc1)NC(=O)c1nccn1-c1ccccc1]

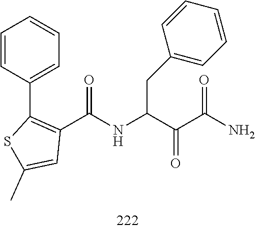 [SMILES: Cc1cc(C(=O)NC(Cc2ccccc2)C(=O)C(N)=O)c(-c2ccccc2)s1]

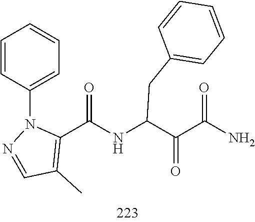 [SMILES: Cc1cnn(-c2ccccc2)c1C(=O)NC(Cc1ccccc1)C(=O)C(N)=O]

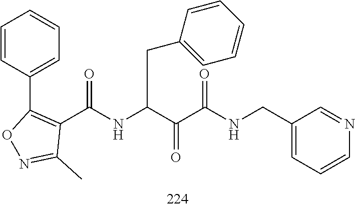 [SMILES: Cc1noc(-c2ccccc2)c1C(=O)NC(Cc1ccccc1)C(=O)C(=O)NCc1cccnc1]

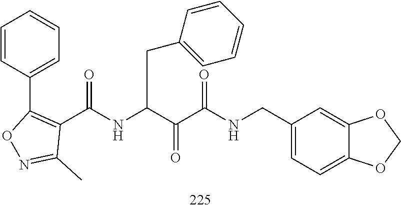 [SMILES: Cc1noc(-c2ccccc2)c1C(=O)NC(Cc1ccccc1)C(=O)C(=O)NCc1ccc2c(c1)OCO2]

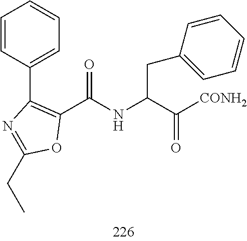 [SMILES: CCc1nc(-c2ccccc2)c(C(=O)NC(Cc2ccccc2)C(=O)C(N)=O)o1]

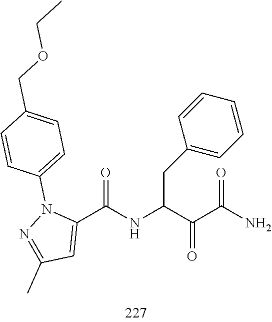 [SMILES: CCOCc1ccc(-n2nc(C)cc2C(=O)NC(Cc2ccccc2)C(=O)C(N)=O)cc1]

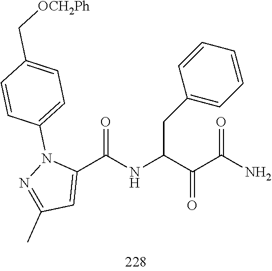

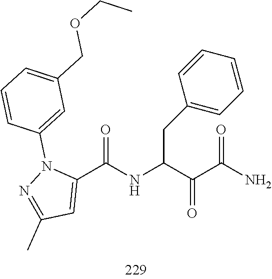 [SMILES: CCOCc1cccc(-n2nc(C)cc2C(=O)NC(Cc2ccccc2)C(=O)C(N)=O)c1]

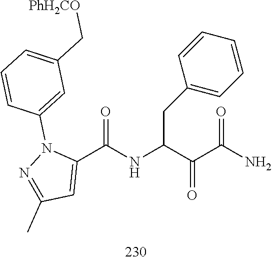

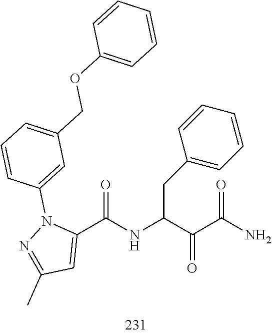 [SMILES: Cc1cc(C(=O)NC(Cc2ccccc2)C(=O)C(N)=O)n(-c2cccc(COc3ccccc3)c2)n1]

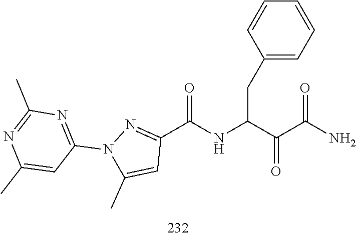 [SMILES: Cc1cc(-n2nc(C(=O)NC(Cc3ccccc3)C(=O)C(N)=O)cc2C)nc(C)n1]

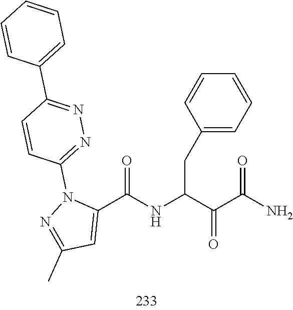 [SMILES: Cc1cc(C(=O)NC(Cc2ccccc2)C(=O)C(N)=O)n(-c2ccc(-c3ccccc3)nn2)n1]

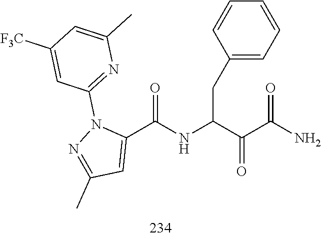 [SMILES: Cc1cc(C(F)(F)F)cc(-n2nc(C)cc2C(=O)NC(Cc2ccccc2)C(=O)C(N)=O)n1]

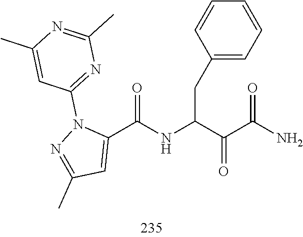 [SMILES: Cc1cc(-n2nc(C)cc2C(=O)NC(Cc2ccccc2)C(=O)C(N)=O)nc(C)n1]

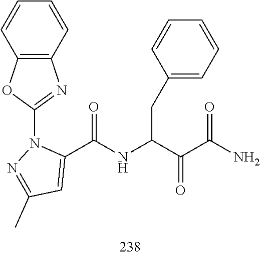 [SMILES: Cc1cc(C(=O)NC(Cc2ccccc2)C(=O)C(N)=O)n(-c2nc3ccccc3o2)n1]

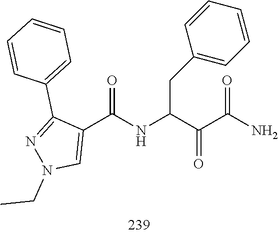 [SMILES: CCn1cc(C(=O)NC(Cc2ccccc2)C(=O)C(N)=O)c(-c2ccccc2)n1]

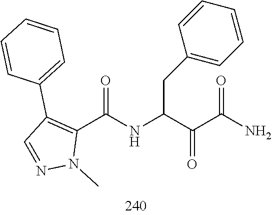 [SMILES: Cn1ncc(-c2ccccc2)c1C(=O)NC(Cc1ccccc1)C(=O)C(N)=O]

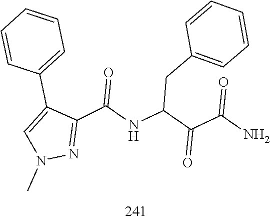 [SMILES: Cn1cc(-c2ccccc2)c(C(=O)NC(Cc2ccccc2)C(=O)C(N)=O)n1]

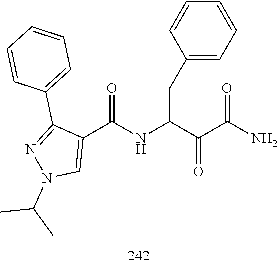 [SMILES: CC(C)n1cc(C(=O)NC(Cc2ccccc2)C(=O)C(N)=O)c(-c2ccccc2)n1]

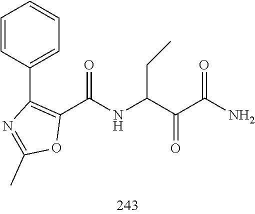 [SMILES: CCC(NC(=O)c1oc(C)nc1-c1ccccc1)C(=O)C(N)=O]

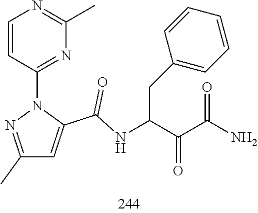 [SMILES: Cc1cc(C(=O)NC(Cc2ccccc2)C(=O)C(N)=O)n(-c2ccnc(C)n2)n1]

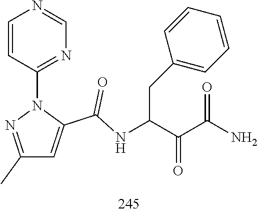 [SMILES: Cc1cc(C(=O)NC(Cc2ccccc2)C(=O)C(N)=O)n(-c2ccncn2)n1]

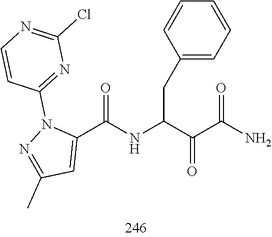 [SMILES: Cc1cc(C(=O)NC(Cc2ccccc2)C(=O)C(N)=O)n(-c2ccnc(Cl)n2)n1]

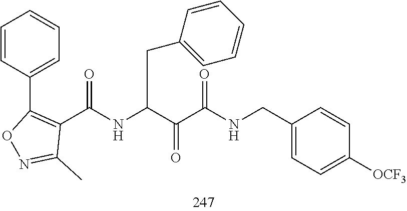 [SMILES: Cc1noc(-c2ccccc2)c1C(=O)NC(Cc1ccccc1)C(=O)C(=O)NCc1ccc(OC(F)(F)F)cc1]

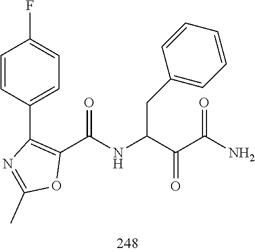 [SMILES: Cc1nc(-c2ccc(F)cc2)c(C(=O)NC(Cc2ccccc2)C(=O)C(N)=O)o1]

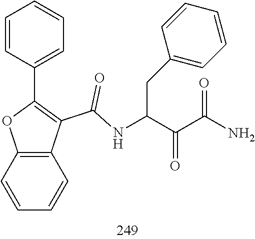 [SMILES: NC(=O)C(=O)C(Cc1ccccc1)NC(=O)c1c(-c2ccccc2)oc2ccccc12]

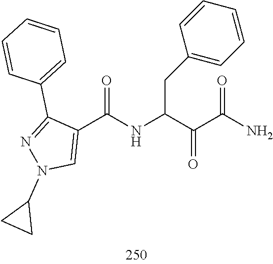 [SMILES: NC(=O)C(=O)C(Cc1ccccc1)NC(=O)c1cn(C2CC2)nc1-c1ccccc1]

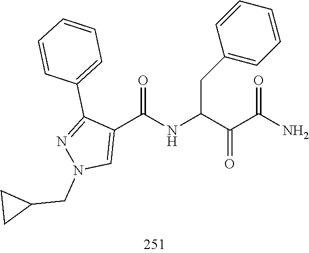 [SMILES: NC(=O)C(=O)C(Cc1ccccc1)NC(=O)c1cn(CC2CC2)nc1-c1ccccc1]

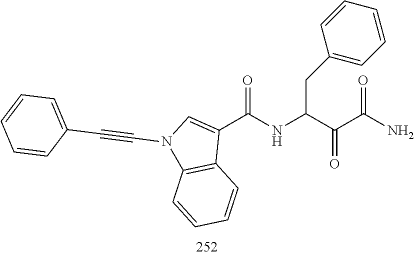 [SMILES: NC(=O)C(=O)C(Cc1ccccc1)NC(=O)c1cn(C#Cc2ccccc2)c2ccccc12]

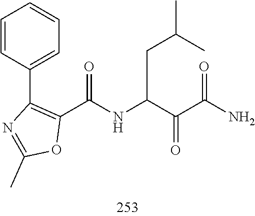 [SMILES: Cc1nc(-c2ccccc2)c(C(=O)NC(CC(C)C)C(=O)C(N)=O)o1]

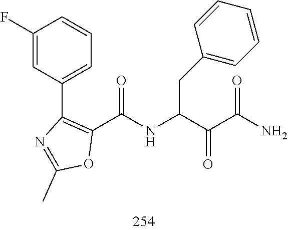 [SMILES: Cc1nc(-c2cccc(F)c2)c(C(=O)NC(Cc2ccccc2)C(=O)C(N)=O)o1]

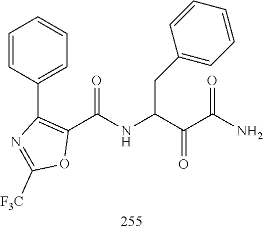 [SMILES: NC(=O)C(=O)C(Cc1ccccc1)NC(=O)c1oc(C(F)(F)F)nc1-c1ccccc1]

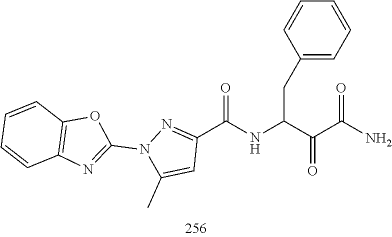 [SMILES: Cc1cc(C(=O)NC(Cc2ccccc2)C(=O)C(N)=O)nn1-c1nc2ccccc2o1]

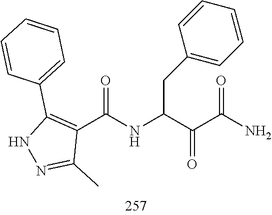 [SMILES: Cc1n[nH]c(-c2ccccc2)c1C(=O)NC(Cc1ccccc1)C(=O)C(N)=O]

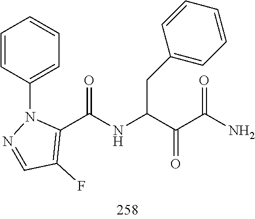 [SMILES: NC(=O)C(=O)C(Cc1ccccc1)NC(=O)c1c(F)cnn1-c1ccccc1]

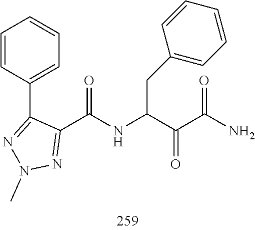 [SMILES: Cn1nc(C(=O)NC(Cc2ccccc2)C(=O)C(N)=O)c(-c2ccccc2)n1]

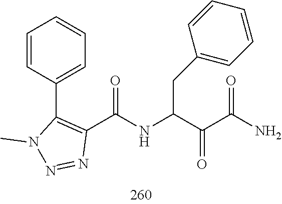 [SMILES: Cn1nnc(C(=O)NC(Cc2ccccc2)C(=O)C(N)=O)c1-c1ccccc1]

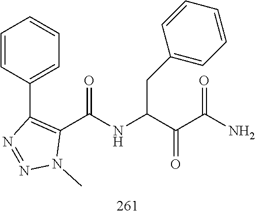 [SMILES: Cn1nnc(-c2ccccc2)c1C(=O)NC(Cc1ccccc1)C(=O)C(N)=O]

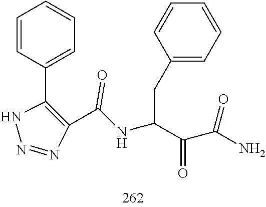 [SMILES: NC(=O)C(=O)C(Cc1ccccc1)NC(=O)c1nn[nH]c1-c1ccccc1]

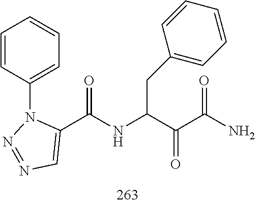 [SMILES: NC(=O)C(=O)C(Cc1ccccc1)NC(=O)c1cnnn1-c1ccccc1]

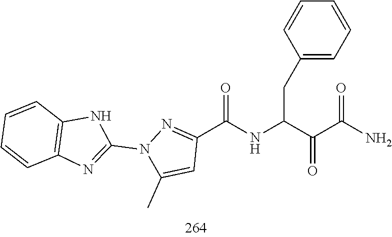 [SMILES: Cc1cc(C(=O)NC(Cc2ccccc2)C(=O)C(N)=O)nn1-c1nc2ccccc2[nH]1]

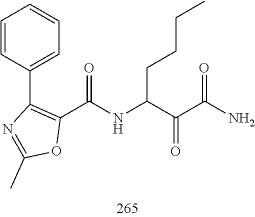 [SMILES: CCCCC(NC(=O)c1oc(C)nc1-c1ccccc1)C(=O)C(N)=O]

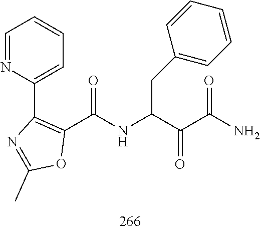 [SMILES: Cc1nc(-c2ccccn2)c(C(=O)NC(Cc2ccccc2)C(=O)C(N)=O)o1]

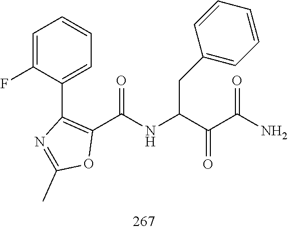 [SMILES: Cc1nc(-c2ccccc2F)c(C(=O)NC(Cc2ccccc2)C(=O)C(N)=O)o1]

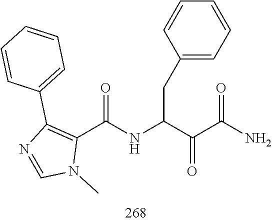 [SMILES: Cn1cnc(-c2ccccc2)c1C(=O)NC(Cc1ccccc1)C(=O)C(N)=O]

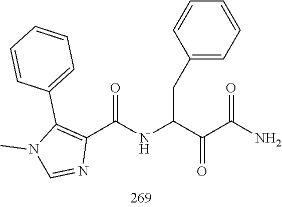 [SMILES: Cn1cnc(C(=O)NC(Cc2ccccc2)C(=O)C(N)=O)c1-c1ccccc1]

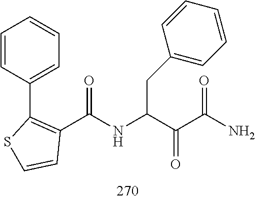 [SMILES: NC(=O)C(=O)C(Cc1ccccc1)NC(=O)c1ccsc1-c1ccccc1]

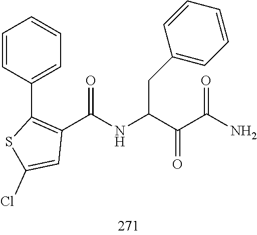 [SMILES: NC(=O)C(=O)C(Cc1ccccc1)NC(=O)c1cc(Cl)sc1-c1ccccc1]

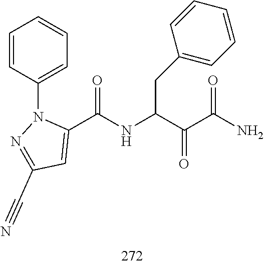 [SMILES: N#Cc1cc(C(=O)NC(Cc2ccccc2)C(=O)C(N)=O)n(-c2ccccc2)n1]

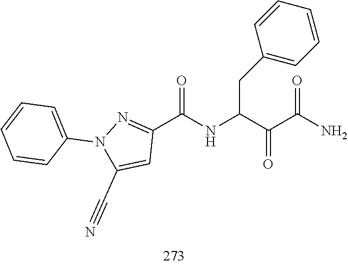 [SMILES: N#Cc1cc(C(=O)NC(Cc2ccccc2)C(=O)C(N)=O)nn1-c1ccccc1]

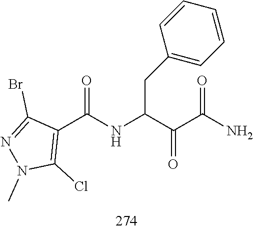 [SMILES: Cn1nc(Br)c(C(=O)NC(Cc2ccccc2)C(=O)C(N)=O)c1Cl]

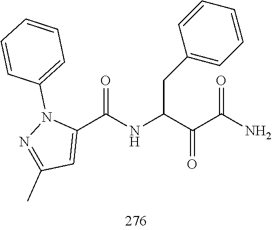 [SMILES: Cc1cc(C(=O)NC(Cc2ccccc2)C(=O)C(N)=O)n(-c2ccccc2)n1]

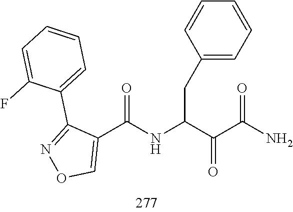 [SMILES: NC(=O)C(=O)C(Cc1ccccc1)NC(=O)c1conc1-c1ccccc1F]

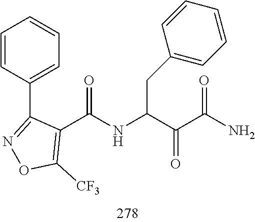 [SMILES: NC(=O)C(=O)C(Cc1ccccc1)NC(=O)c1c(-c2ccccc2)noc1C(F)(F)F]

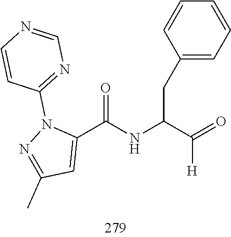 [SMILES: Cc1cc(C(=O)NC(C=O)Cc2ccccc2)n(-c2ccncn2)n1]

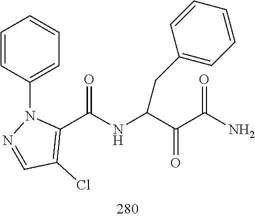 [SMILES: NC(=O)C(=O)C(Cc1ccccc1)NC(=O)c1c(Cl)cnn1-c1ccccc1]

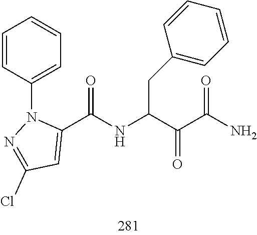 [SMILES: NC(=O)C(=O)C(Cc1ccccc1)NC(=O)c1cc(Cl)nn1-c1ccccc1]

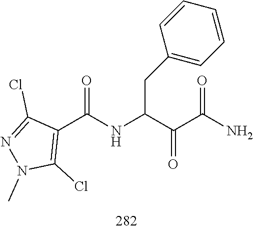 [SMILES: Cn1nc(Cl)c(C(=O)NC(Cc2ccccc2)C(=O)C(N)=O)c1Cl]

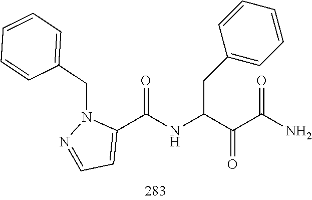 [SMILES: NC(=O)C(=O)C(Cc1ccccc1)NC(=O)c1ccnn1Cc1ccccc1]

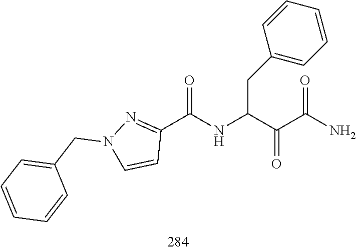 [SMILES: NC(=O)C(=O)C(Cc1ccccc1)NC(=O)c1ccn(Cc2ccccc2)n1]

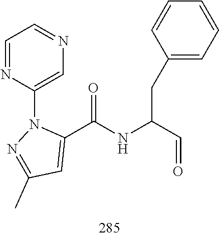 [SMILES: Cc1cc(C(=O)NC(C=O)Cc2ccccc2)n(-c2cnccn2)n1]

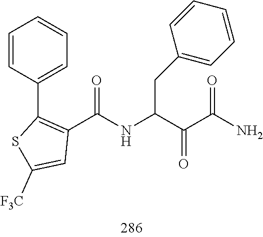 [SMILES: NC(=O)C(=O)C(Cc1ccccc1)NC(=O)c1cc(C(F)(F)F)sc1-c1ccccc1]

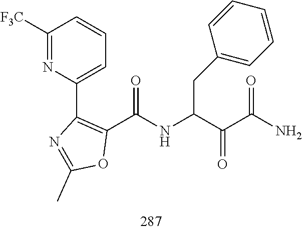 [SMILES: Cc1nc(-c2cccc(C(F)(F)F)n2)c(C(=O)NC(Cc2ccccc2)C(=O)C(N)=O)o1]

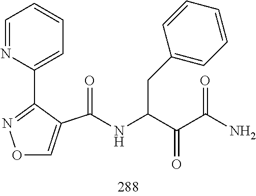 [SMILES: NC(=O)C(=O)C(Cc1ccccc1)NC(=O)c1conc1-c1ccccn1]

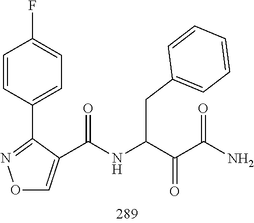 [SMILES: NC(=O)C(=O)C(Cc1ccccc1)NC(=O)c1conc1-c1ccc(F)cc1]

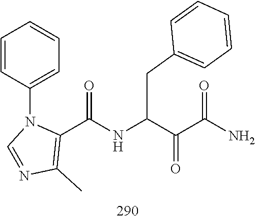 [SMILES: Cc1ncn(-c2ccccc2)c1C(=O)NC(Cc1ccccc1)C(=O)C(N)=O]

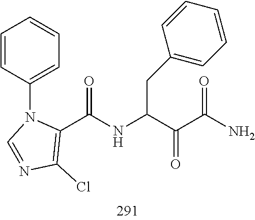 [SMILES: NC(=O)C(=O)C(Cc1ccccc1)NC(=O)c1c(Cl)ncn1-c1ccccc1]

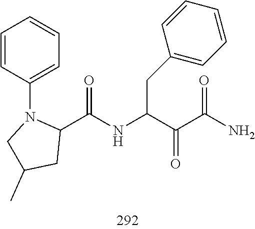 [SMILES: CC1CC(C(=O)NC(Cc2ccccc2)C(=O)C(N)=O)N(c2ccccc2)C1]

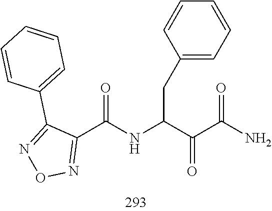 [SMILES: NC(=O)C(=O)C(Cc1ccccc1)NC(=O)c1nonc1-c1ccccc1]

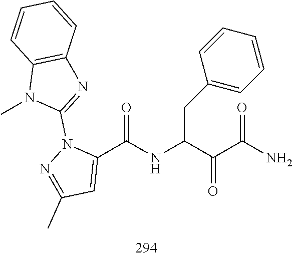 [SMILES: Cc1cc(C(=O)NC(Cc2ccccc2)C(=O)C(N)=O)n(-c2nc3ccccc3n2C)n1]

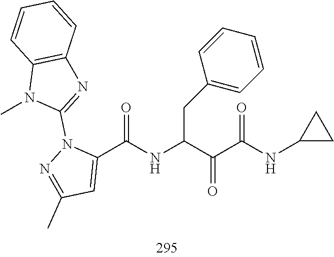 [SMILES: Cc1cc(C(=O)NC(Cc2ccccc2)C(=O)C(=O)NC2CC2)n(-c2nc3ccccc3n2C)n1]

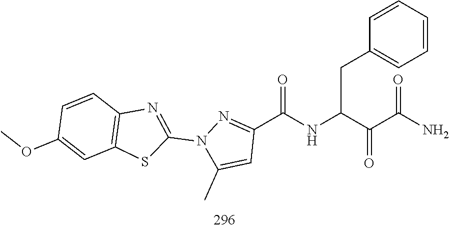 [SMILES: COc1ccc2nc(-n3nc(C(=O)NC(Cc4ccccc4)C(=O)C(N)=O)cc3C)sc2c1]

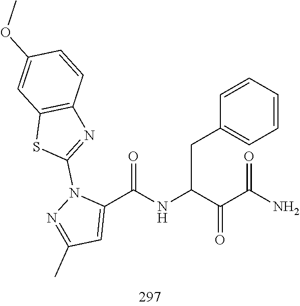 [SMILES: COc1ccc2nc(-n3nc(C)cc3C(=O)NC(Cc3ccccc3)C(=O)C(N)=O)sc2c1]

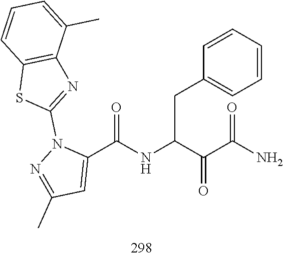 [SMILES: Cc1cc(C(=O)NC(Cc2ccccc2)C(=O)C(N)=O)n(-c2nc3c(C)cccc3s2)n1]

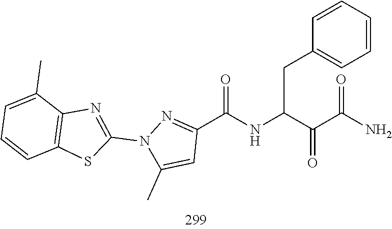 [SMILES: Cc1cccc2sc(-n3nc(C(=O)NC(Cc4ccccc4)C(=O)C(N)=O)cc3C)nc12]

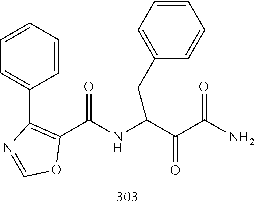 [SMILES: NC(=O)C(=O)C(Cc1ccccc1)NC(=O)c1ocnc1-c1ccccc1]

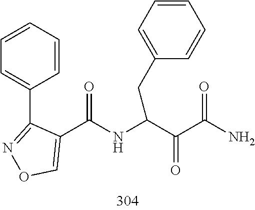 [SMILES: NC(=O)C(=O)C(Cc1ccccc1)NC(=O)c1conc1-c1ccccc1]

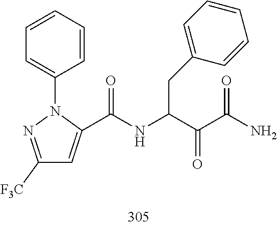 [SMILES: NC(=O)C(=O)C(Cc1ccccc1)NC(=O)c1cc(C(F)(F)F)nn1-c1ccccc1]

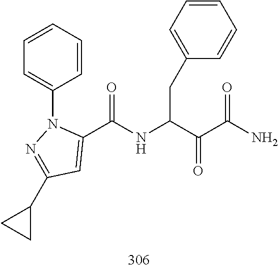 [SMILES: NC(=O)C(=O)C(Cc1ccccc1)NC(=O)c1cc(C2CC2)nn1-c1ccccc1]

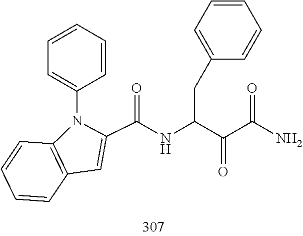 [SMILES: NC(=O)C(=O)C(Cc1ccccc1)NC(=O)c1cc2ccccc2n1-c1ccccc1]

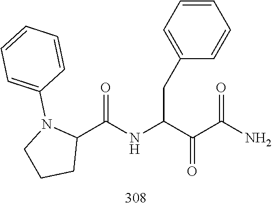 [SMILES: NC(=O)C(=O)C(Cc1ccccc1)NC(=O)C1CCCN1c1ccccc1]

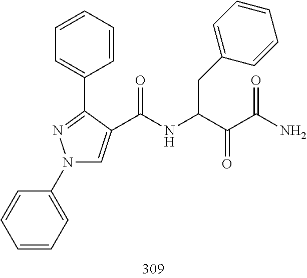 [SMILES: NC(=O)C(=O)C(Cc1ccccc1)NC(=O)c1cn(-c2ccccc2)nc1-c1ccccc1]

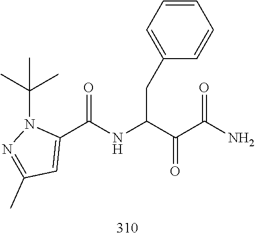 [SMILES: Cc1cc(C(=O)NC(Cc2ccccc2)C(=O)C(N)=O)n(C(C)(C)C)n1]

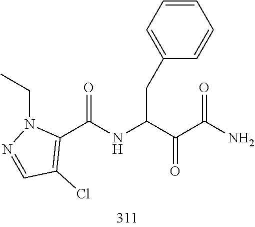 [SMILES: CCn1ncc(Cl)c1C(=O)NC(Cc1ccccc1)C(=O)C(N)=O]

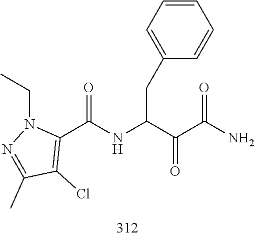 [SMILES: CCn1nc(C)c(Cl)c1C(=O)NC(Cc1ccccc1)C(=O)C(N)=O]

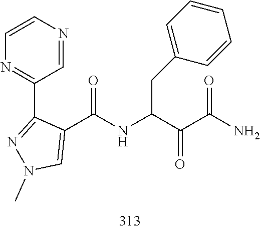 [SMILES: Cn1cc(C(=O)NC(Cc2ccccc2)C(=O)C(N)=O)c(-c2cnccn2)n1]

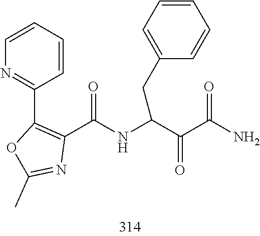 [SMILES: Cc1nc(C(=O)NC(Cc2ccccc2)C(=O)C(N)=O)c(-c2ccccn2)o1]

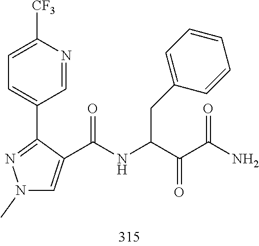 [SMILES: Cn1cc(C(=O)NC(Cc2ccccc2)C(=O)C(N)=O)c(-c2ccc(C(F)(F)F)nc2)n1]

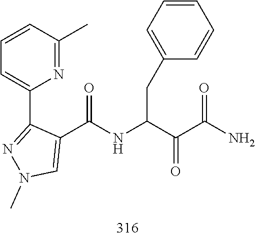 [SMILES: Cc1cccc(-c2nn(C)cc2C(=O)NC(Cc2ccccc2)C(=O)C(N)=O)n1]

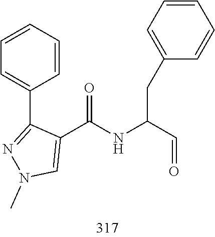 [SMILES: Cn1cc(C(=O)NC(C=O)Cc2ccccc2)c(-c2ccccc2)n1]

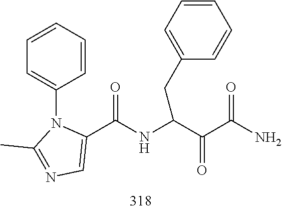 [SMILES: Cc1ncc(C(=O)NC(Cc2ccccc2)C(=O)C(N)=O)n1-c1ccccc1]

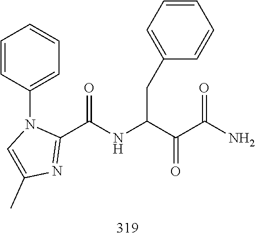 [SMILES: Cc1cn(-c2ccccc2)c(C(=O)NC(Cc2ccccc2)C(=O)C(N)=O)n1]

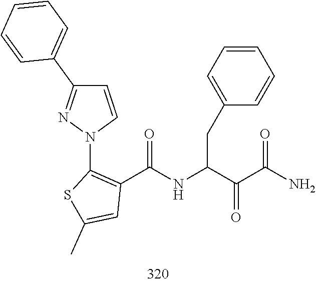 [SMILES: Cc1cc(C(=O)NC(Cc2ccccc2)C(=O)C(N)=O)c(-n2ccc(-c3ccccc3)n2)s1]

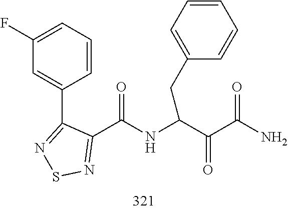 [SMILES: NC(=O)C(=O)C(Cc1ccccc1)NC(=O)c1nsnc1-c1cccc(F)c1]

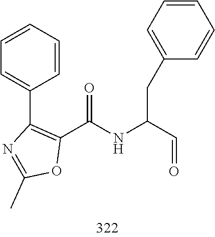 [SMILES: Cc1nc(-c2ccccc2)c(C(=O)NC(C=O)Cc2ccccc2)o1]

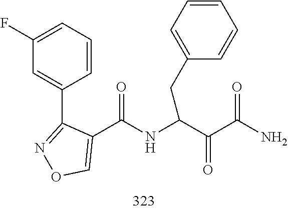 [SMILES: NC(=O)C(=O)C(Cc1ccccc1)NC(=O)c1conc1-c1cccc(F)c1]

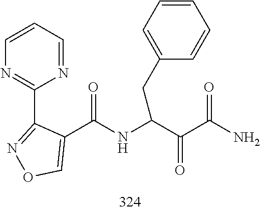 [SMILES: NC(=O)C(=O)C(Cc1ccccc1)NC(=O)c1conc1-c1ncccn1]

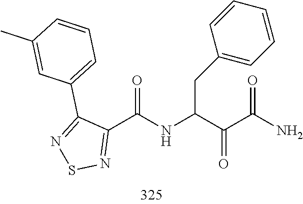 [SMILES: Cc1cccc(-c2nsnc2C(=O)NC(Cc2ccccc2)C(=O)C(N)=O)c1]

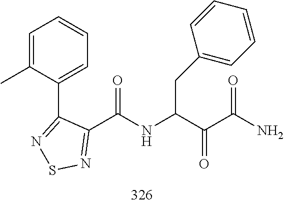 [SMILES: Cc1ccccc1-c1nsnc1C(=O)NC(Cc1ccccc1)C(=O)C(N)=O]

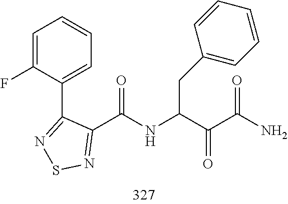 [SMILES: NC(=O)C(=O)C(Cc1ccccc1)NC(=O)c1nsnc1-c1ccccc1F]

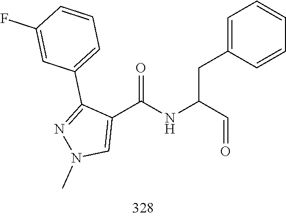 [SMILES: Cn1cc(C(=O)NC(C=O)Cc2ccccc2)c(-c2cccc(F)c2)n1]

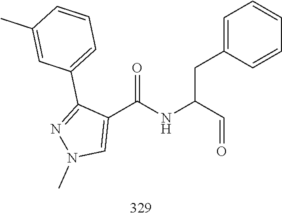 [SMILES: Cc1cccc(-c2nn(C)cc2C(=O)NC(C=O)Cc2ccccc2)c1]

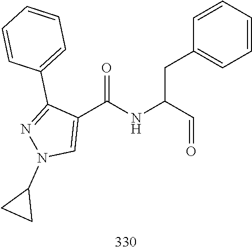 [SMILES: O=CC(Cc1ccccc1)NC(=O)c1cn(C2CC2)nc1-c1ccccc1]

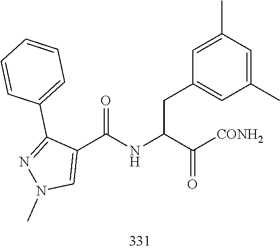 [SMILES: Cc1cc(C)cc(CC(NC(=O)c2cn(C)nc2-c2ccccc2)C(=O)C(N)=O)c1]

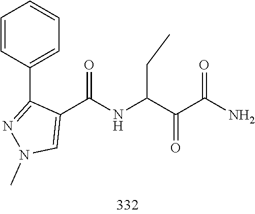 [SMILES: CCC(NC(=O)c1cn(C)nc1-c1ccccc1)C(=O)C(N)=O]

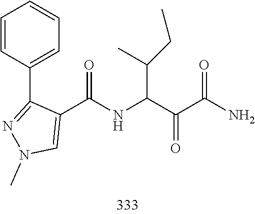 [SMILES: CCC(C)C(NC(=O)c1cn(C)nc1-c1ccccc1)C(=O)C(N)=O]

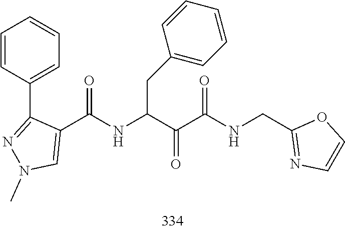 [SMILES: Cn1cc(C(=O)NC(Cc2ccccc2)C(=O)C(=O)NCc2ncco2)c(-c2ccccc2)n1]

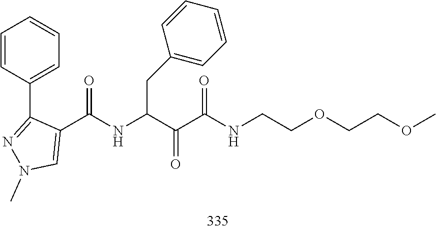 [SMILES: COCCOCCNC(=O)C(=O)C(Cc1ccccc1)NC(=O)c1cn(C)nc1-c1ccccc1]

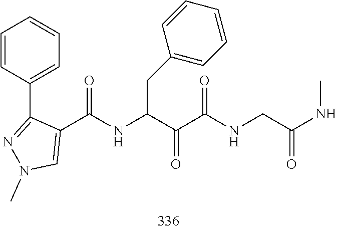 [SMILES: CNC(=O)CNC(=O)C(=O)C(Cc1ccccc1)NC(=O)c1cn(C)nc1-c1ccccc1]

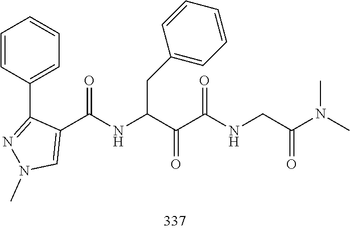 [SMILES: CN(C)C(=O)CNC(=O)C(=O)C(Cc1ccccc1)NC(=O)c1cn(C)nc1-c1ccccc1]

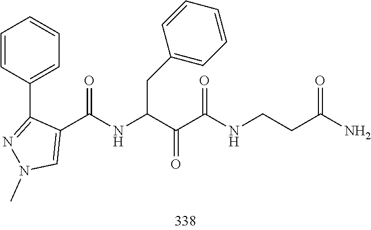 [SMILES: Cn1cc(C(=O)NC(Cc2ccccc2)C(=O)C(=O)NCCC(N)=O)c(-c2ccccc2)n1]

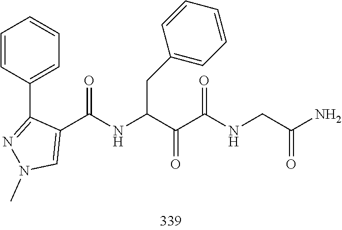 [SMILES: Cn1cc(C(=O)NC(Cc2ccccc2)C(=O)C(=O)NCC(N)=O)c(-c2ccccc2)n1]

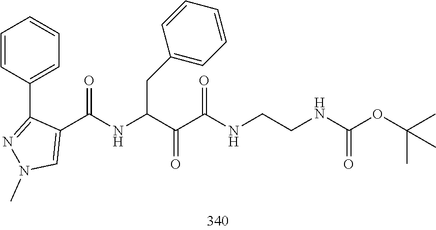 [SMILES: Cn1cc(C(=O)NC(Cc2ccccc2)C(=O)C(=O)NCCNC(=O)OC(C)(C)C)c(-c2ccccc2)n1]

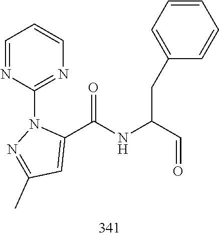 [SMILES: Cc1cc(C(=O)NC(C=O)Cc2ccccc2)n(-c2ncccn2)n1]

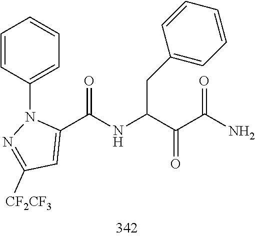

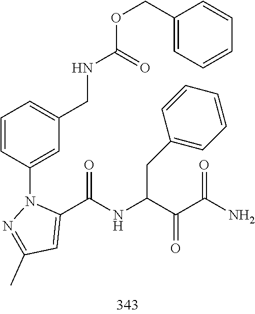 [SMILES: Cc1cc(C(=O)NC(Cc2ccccc2)C(=O)C(N)=O)n(-c2cccc(CNC(=O)OCc3ccccc3)c2)n1]

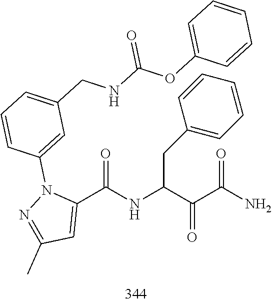 [SMILES: Cc1cc(C(=O)NC(Cc2ccccc2)C(=O)C(N)=O)n(-c2cccc(CNC(=O)Oc3ccccc3)c2)n1]

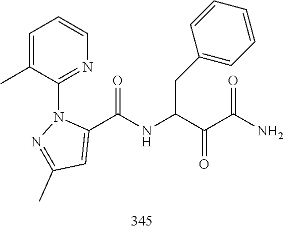 [SMILES: Cc1cc(C(=O)NC(Cc2ccccc2)C(=O)C(N)=O)n(-c2ncccc2C)n1]

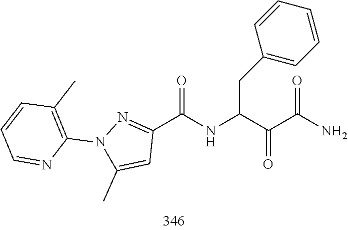 [SMILES: Cc1cccnc1-n1nc(C(=O)NC(Cc2ccccc2)C(=O)C(N)=O)cc1C]

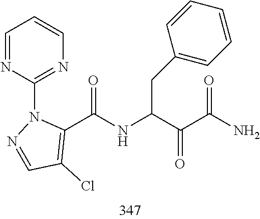 [SMILES: NC(=O)C(=O)C(Cc1ccccc1)NC(=O)c1c(Cl)cnn1-c1ncccn1]

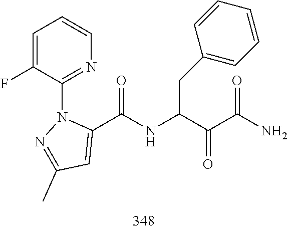 [SMILES: Cc1cc(C(=O)NC(Cc2ccccc2)C(=O)C(N)=O)n(-c2ncccc2F)n1]

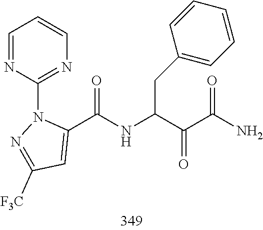 [SMILES: NC(=O)C(=O)C(Cc1ccccc1)NC(=O)c1cc(C(F)(F)F)nn1-c1ncccn1]

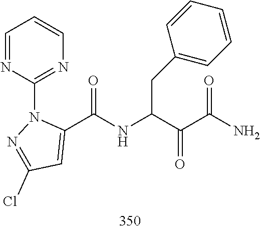 [SMILES: NC(=O)C(=O)C(Cc1ccccc1)NC(=O)c1cc(Cl)nn1-c1ncccn1]

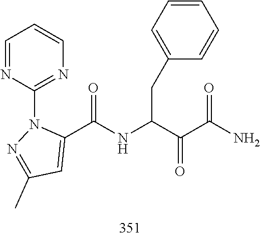 [SMILES: Cc1cc(C(=O)NC(Cc2ccccc2)C(=O)C(N)=O)n(-c2ncccn2)n1]

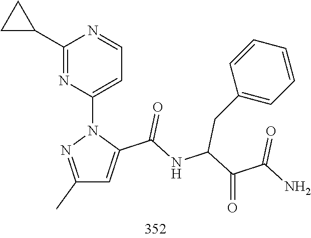 [SMILES: Cc1cc(C(=O)NC(Cc2ccccc2)C(=O)C(N)=O)n(-c2ccnc(C3CC3)n2)n1]

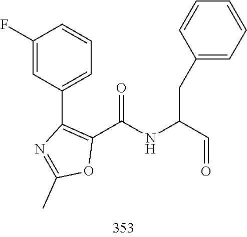 [SMILES: Cc1nc(-c2cccc(F)c2)c(C(=O)NC(C=O)Cc2ccccc2)o1]

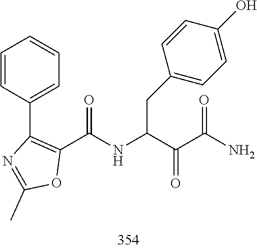 [SMILES: Cc1nc(-c2ccccc2)c(C(=O)NC(Cc2ccc(O)cc2)C(=O)C(N)=O)o1]

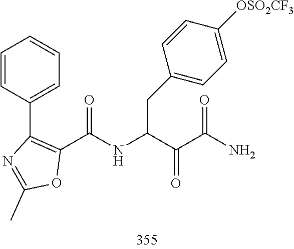

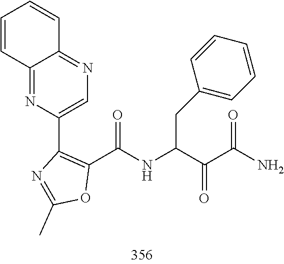 [SMILES: Cc1nc(-c2cnc3ccccc3n2)c(C(=O)NC(Cc2ccccc2)C(=O)C(N)=O)o1]

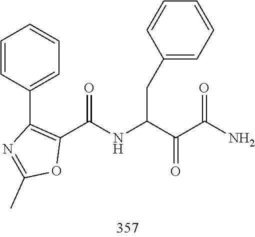 [SMILES: Cc1nc(-c2ccccc2)c(C(=O)NC(Cc2ccccc2)C(=O)C(N)=O)o1]

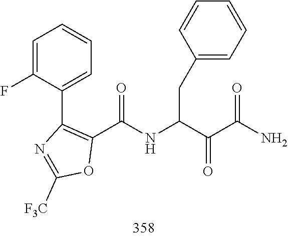 [SMILES: NC(=O)C(=O)C(Cc1ccccc1)NC(=O)c1oc(C(F)(F)F)nc1-c1ccccc1F]

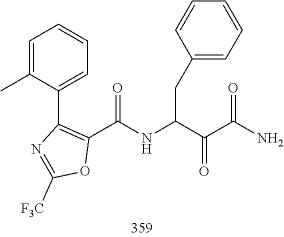 [SMILES: Cc1ccccc1-c1nc(C(F)(F)F)oc1C(=O)NC(Cc1ccccc1)C(=O)C(N)=O]

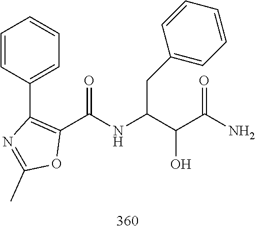 [SMILES: Cc1nc(-c2ccccc2)c(C(=O)NC(Cc2ccccc2)C(O)C(N)=O)o1]

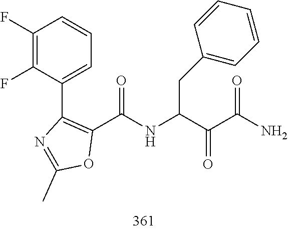 [SMILES: Cc1nc(-c2cccc(F)c2F)c(C(=O)NC(Cc2ccccc2)C(=O)C(N)=O)o1]

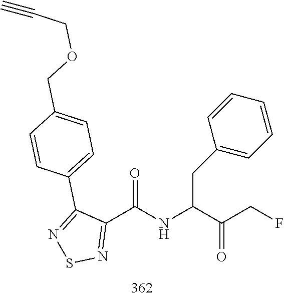 [SMILES: C#CCOCc1ccc(-c2nsnc2C(=O)NC(Cc2ccccc2)C(=O)CF)cc1]

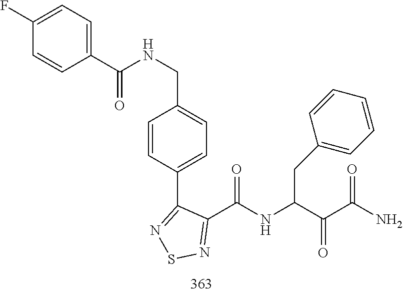 [SMILES: NC(=O)C(=O)C(Cc1ccccc1)NC(=O)c1nsnc1-c1ccc(CNC(=O)c2ccc(F)cc2)cc1]

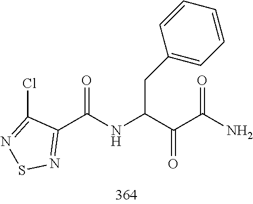 [SMILES: NC(=O)C(=O)C(Cc1ccccc1)NC(=O)c1nsnc1Cl]

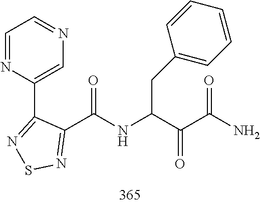 [SMILES: NC(=O)C(=O)C(Cc1ccccc1)NC(=O)c1nsnc1-c1cnccn1]

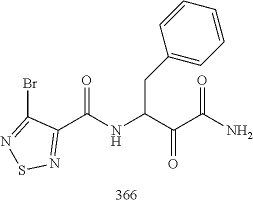 [SMILES: NC(=O)C(=O)C(Cc1ccccc1)NC(=O)c1nsnc1Br]

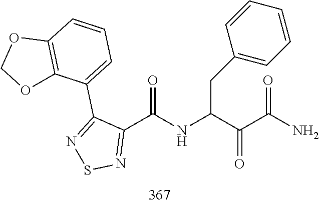 [SMILES: NC(=O)C(=O)C(Cc1ccccc1)NC(=O)c1nsnc1-c1cccc2c1OCO2]

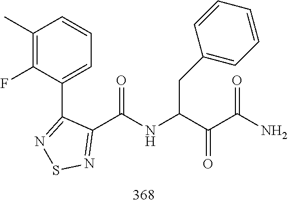 [SMILES: Cc1cccc(-c2nsnc2C(=O)NC(Cc2ccccc2)C(=O)C(N)=O)c1F]

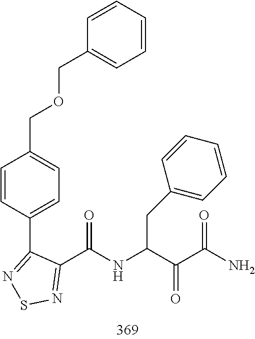 [SMILES: NC(=O)C(=O)C(Cc1ccccc1)NC(=O)c1nsnc1-c1ccc(COCc2ccccc2)cc1]

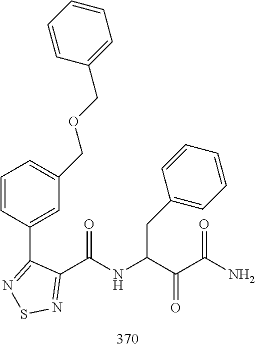 [SMILES: NC(=O)C(=O)C(Cc1ccccc1)NC(=O)c1nsnc1-c1cccc(COCc2ccccc2)c1]

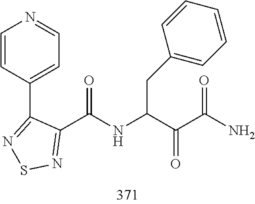 [SMILES: NC(=O)C(=O)C(Cc1ccccc1)NC(=O)c1nsnc1-c1ccncc1]

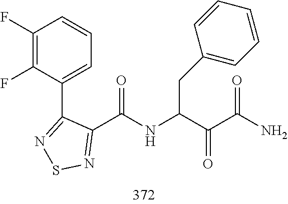 [SMILES: NC(=O)C(=O)C(Cc1ccccc1)NC(=O)c1nsnc1-c1cccc(F)c1F]

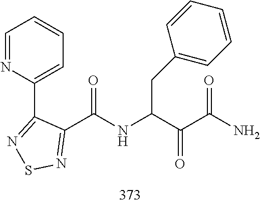 [SMILES: NC(=O)C(=O)C(Cc1ccccc1)NC(=O)c1nsnc1-c1ccccn1]

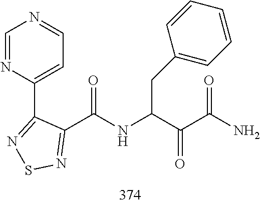 [SMILES: NC(=O)C(=O)C(Cc1ccccc1)NC(=O)c1nsnc1-c1ccncn1]

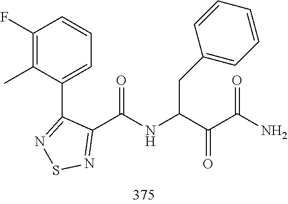 [SMILES: Cc1c(F)cccc1-c1nsnc1C(=O)NC(Cc1ccccc1)C(=O)C(N)=O]

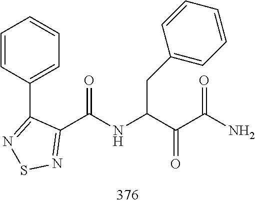 [SMILES: NC(=O)C(=O)C(Cc1ccccc1)NC(=O)c1nsnc1-c1ccccc1]

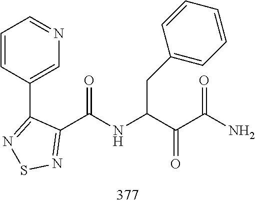 [SMILES: NC(=O)C(=O)C(Cc1ccccc1)NC(=O)c1nsnc1-c1cccnc1]

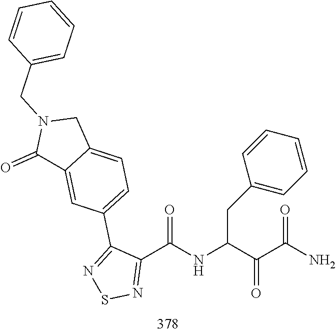 [SMILES: NC(=O)C(=O)C(Cc1ccccc1)NC(=O)c1nsnc1-c1ccc2c(c1)C(=O)N(Cc1ccccc1)C2]

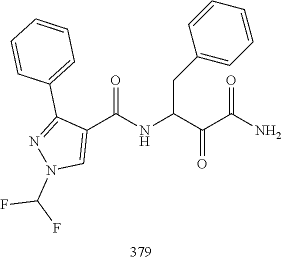 [SMILES: NC(=O)C(=O)C(Cc1ccccc1)NC(=O)c1cn(C(F)F)nc1-c1ccccc1]

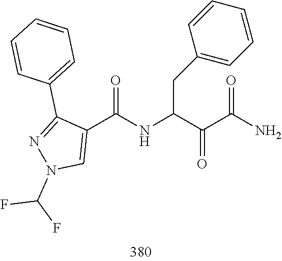 [SMILES: NC(=O)C(=O)C(Cc1ccccc1)NC(=O)c1cn(C(F)F)nc1-c1ccccc1]

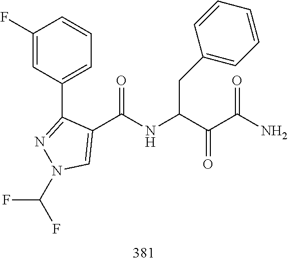 [SMILES: NC(=O)C(=O)C(Cc1ccccc1)NC(=O)c1cn(C(F)F)nc1-c1cccc(F)c1]

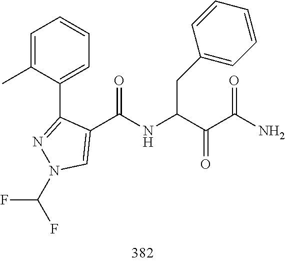 [SMILES: Cc1ccccc1-c1nn(C(F)F)cc1C(=O)NC(Cc1ccccc1)C(=O)C(N)=O]

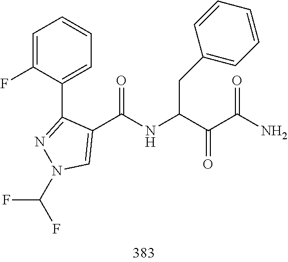 [SMILES: NC(=O)C(=O)C(Cc1ccccc1)NC(=O)c1cn(C(F)F)nc1-c1ccccc1F]

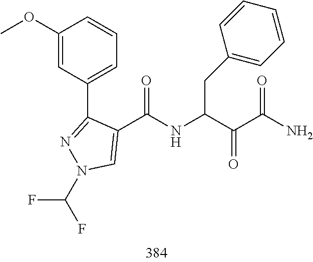 [SMILES: COc1cccc(-c2nn(C(F)F)cc2C(=O)NC(Cc2ccccc2)C(=O)C(N)=O)c1]

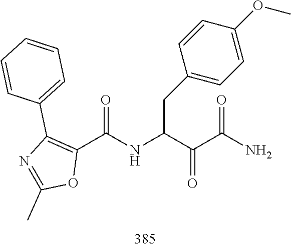 [SMILES: COc1ccc(CC(NC(=O)c2oc(C)nc2-c2ccccc2)C(=O)C(N)=O)cc1]

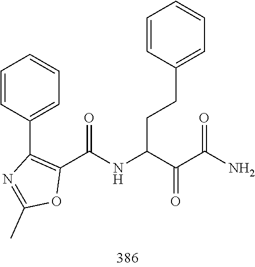 [SMILES: Cc1nc(-c2ccccc2)c(C(=O)NC(CCc2ccccc2)C(=O)C(N)=O)o1]

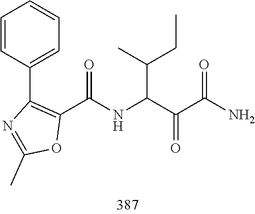 [SMILES: CCC(C)C(NC(=O)c1oc(C)nc1-c1ccccc1)C(=O)C(N)=O]

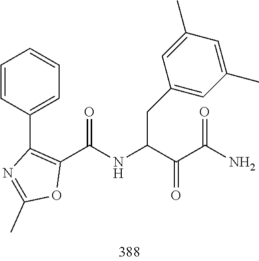 [SMILES: Cc1cc(C)cc(CC(NC(=O)c2oc(C)nc2-c2ccccc2)C(=O)C(N)=O)c1]

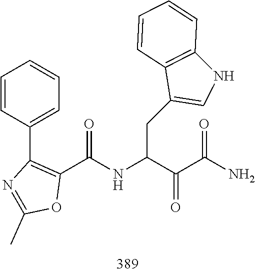 [SMILES: Cc1nc(-c2ccccc2)c(C(=O)NC(Cc2c[nH]c3ccccc23)C(=O)C(N)=O)o1]

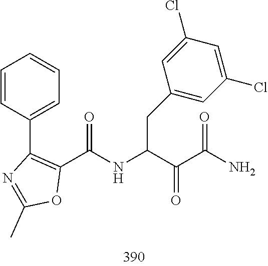 [SMILES: Cc1nc(-c2ccccc2)c(C(=O)NC(Cc2cc(Cl)cc(Cl)c2)C(=O)C(N)=O)o1]

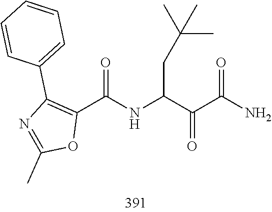 [SMILES: Cc1nc(-c2ccccc2)c(C(=O)NC(CC(C)(C)C)C(=O)C(N)=O)o1]

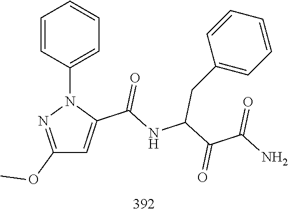 [SMILES: COc1cc(C(=O)NC(Cc2ccccc2)C(=O)C(N)=O)n(-c2ccccc2)n1]

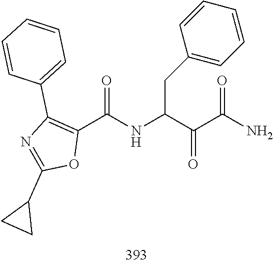 [SMILES: NC(=O)C(=O)C(Cc1ccccc1)NC(=O)c1oc(C2CC2)nc1-c1ccccc1]

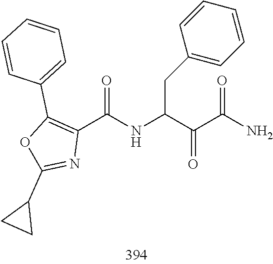 [SMILES: NC(=O)C(=O)C(Cc1ccccc1)NC(=O)c1nc(C2CC2)oc1-c1ccccc1]

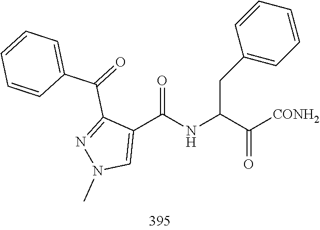 [SMILES: Cn1cc(C(=O)NC(Cc2ccccc2)C(=O)C(N)=O)c(C(=O)c2ccccc2)n1]

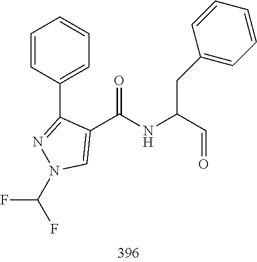 [SMILES: O=CC(Cc1ccccc1)NC(=O)c1cn(C(F)F)nc1-c1ccccc1]

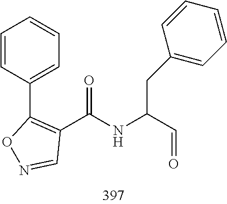 [SMILES: O=CC(Cc1ccccc1)NC(=O)c1cnoc1-c1ccccc1]

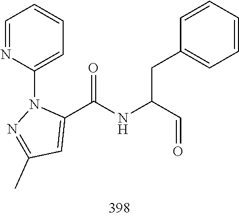 [SMILES: Cc1cc(C(=O)NC(C=O)Cc2ccccc2)n(-c2ccccn2)n1]

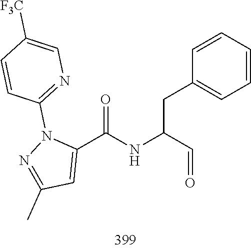 [SMILES: Cc1cc(C(=O)NC(C=O)Cc2ccccc2)n(-c2ccc(C(F)(F)F)cn2)n1]

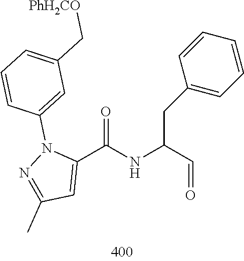

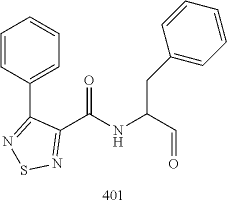 [SMILES: O=CC(Cc1ccccc1)NC(=O)c1nsnc1-c1ccccc1]

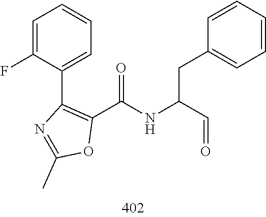 [SMILES: Cc1nc(-c2ccccc2F)c(C(=O)NC(C=O)Cc2ccccc2)o1]

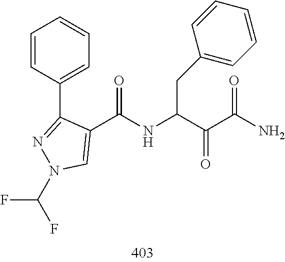 [SMILES: NC(=O)C(=O)C(Cc1ccccc1)NC(=O)c1cn(C(F)F)nc1-c1ccccc1]

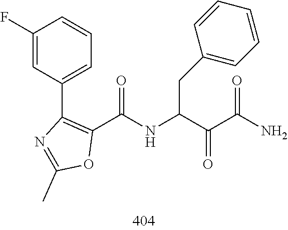 [SMILES: Cc1nc(-c2cccc(F)c2)c(C(=O)NC(Cc2ccccc2)C(=O)C(N)=O)o1]

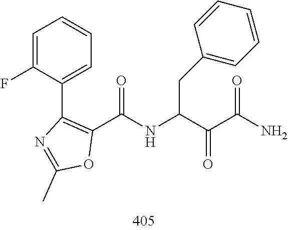 [SMILES: Cc1nc(-c2ccccc2F)c(C(=O)NC(Cc2ccccc2)C(=O)C(N)=O)o1]

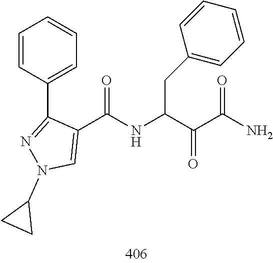 [SMILES: NC(=O)C(=O)C(Cc1ccccc1)NC(=O)c1cn(C2CC2)nc1-c1ccccc1]

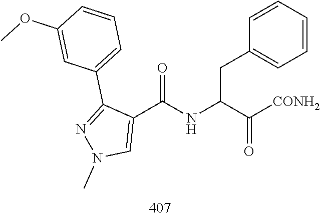 [SMILES: COc1cccc(-c2nn(C)cc2C(=O)NC(Cc2ccccc2)C(=O)C(N)=O)c1]

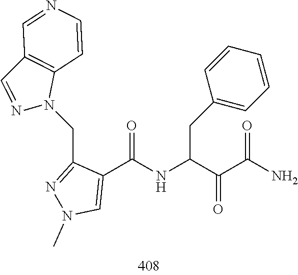 [SMILES: Cn1cc(C(=O)NC(Cc2ccccc2)C(=O)C(N)=O)c(Cn2ncc3cnccc32)n1]

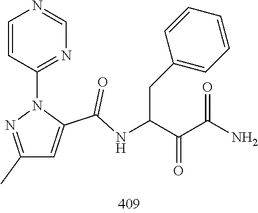 [SMILES: Cc1cc(C(=O)NC(Cc2ccccc2)C(=O)C(N)=O)n(-c2ccncn2)n1]

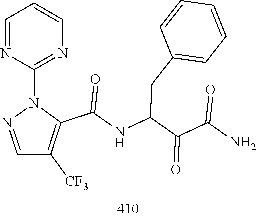 [SMILES: NC(=O)C(=O)C(Cc1ccccc1)NC(=O)c1c(C(F)(F)F)cnn1-c1ncccn1]

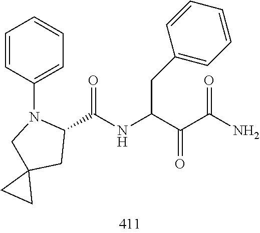 [SMILES: NC(=O)C(=O)C(Cc1ccccc1)NC(=O)[C@@H]1CC2(CC2)CN1c1ccccc1]

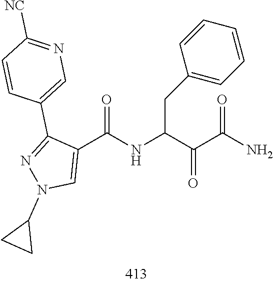 [SMILES: N#Cc1ccc(-c2nn(C3CC3)cc2C(=O)NC(Cc2ccccc2)C(=O)C(N)=O)cn1]

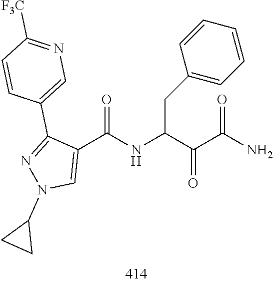 [SMILES: NC(=O)C(=O)C(Cc1ccccc1)NC(=O)c1cn(C2CC2)nc1-c1ccc(C(F)(F)F)nc1]

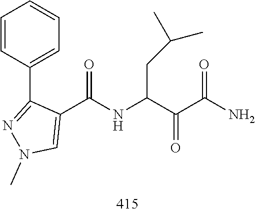 [SMILES: CC(C)CC(NC(=O)c1cn(C)nc1-c1ccccc1)C(=O)C(N)=O]

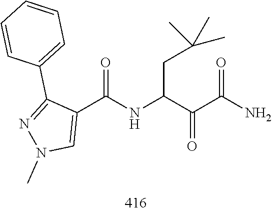 [SMILES: Cn1cc(C(=O)NC(CC(C)(C)C)C(=O)C(N)=O)c(-c2ccccc2)n1]

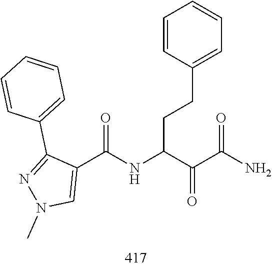 [SMILES: Cn1cc(C(=O)NC(CCc2ccccc2)C(=O)C(N)=O)c(-c2ccccc2)n1]

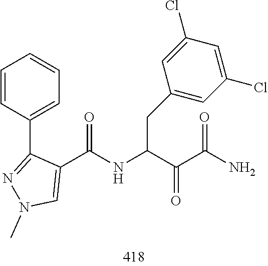 [SMILES: Cn1cc(C(=O)NC(Cc2cc(Cl)cc(Cl)c2)C(=O)C(N)=O)c(-c2ccccc2)n1]

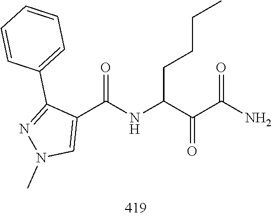 [SMILES: CCCCC(NC(=O)c1cn(C)nc1-c1ccccc1)C(=O)C(N)=O]

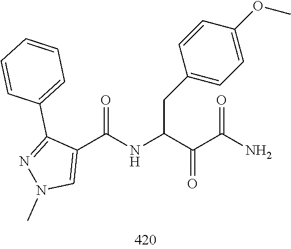 [SMILES: COc1ccc(CC(NC(=O)c2cn(C)nc2-c2ccccc2)C(=O)C(N)=O)cc1]

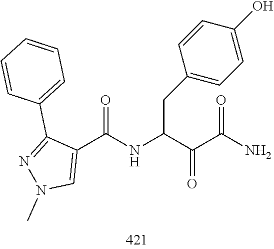 [SMILES: Cn1cc(C(=O)NC(Cc2ccc(O)cc2)C(=O)C(N)=O)c(-c2ccccc2)n1]

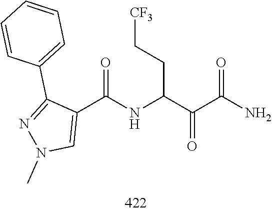 [SMILES: Cn1cc(C(=O)NC(CCC(F)(F)F)C(=O)C(N)=O)c(-c2ccccc2)n1]

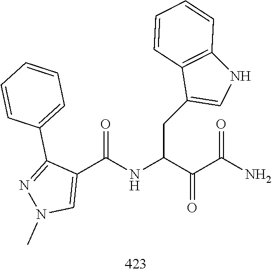 [SMILES: Cn1cc(C(=O)NC(Cc2c[nH]c3ccccc23)C(=O)C(N)=O)c(-c2ccccc2)n1]

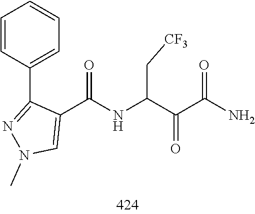 [SMILES: Cn1cc(C(=O)NC(CC(F)(F)F)C(=O)C(N)=O)c(-c2ccccc2)n1]

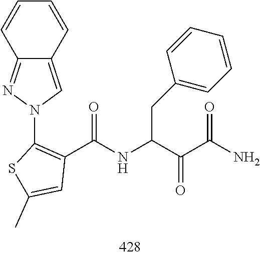 [SMILES: Cc1cc(C(=O)NC(Cc2ccccc2)C(=O)C(N)=O)c(-n2cc3ccccc3n2)s1]

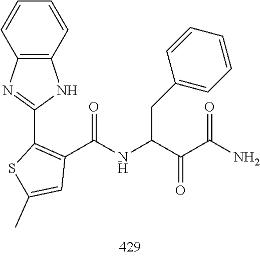 [SMILES: Cc1cc(C(=O)NC(Cc2ccccc2)C(=O)C(N)=O)c(-c2nc3ccccc3[nH]2)s1]

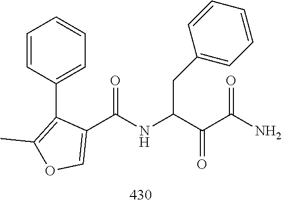 [SMILES: Cc1occ(C(=O)NC(Cc2ccccc2)C(=O)C(N)=O)c1-c1ccccc1]

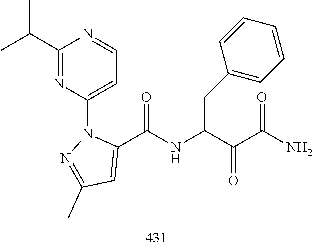 [SMILES: Cc1cc(C(=O)NC(Cc2ccccc2)C(=O)C(N)=O)n(-c2ccnc(C(C)C)n2)n1]

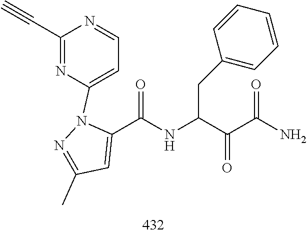 [SMILES: C#Cc1nccc(-n2nc(C)cc2C(=O)NC(Cc2ccccc2)C(=O)C(N)=O)n1]

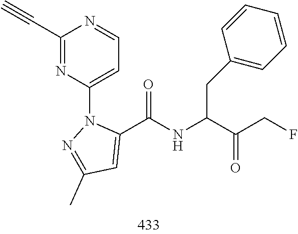 [SMILES: C#Cc1nccc(-n2nc(C)cc2C(=O)NC(Cc2ccccc2)C(=O)CF)n1]

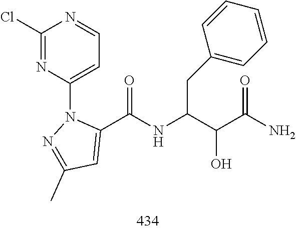 [SMILES: Cc1cc(C(=O)NC(Cc2ccccc2)C(O)C(N)=O)n(-c2ccnc(Cl)n2)n1]

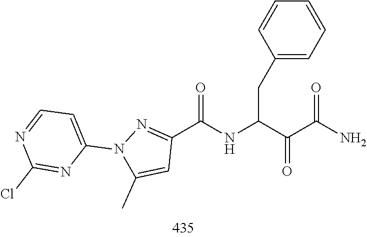 [SMILES: Cc1cc(C(=O)NC(Cc2ccccc2)C(=O)C(N)=O)nn1-c1ccnc(Cl)n1]

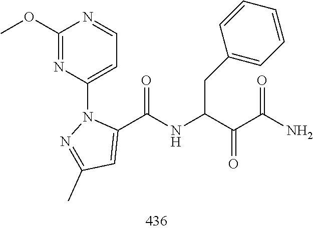 [SMILES: COc1nccc(-n2nc(C)cc2C(=O)NC(Cc2ccccc2)C(=O)C(N)=O)n1]

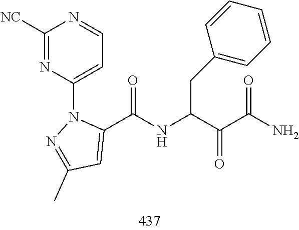 [SMILES: Cc1cc(C(=O)NC(Cc2ccccc2)C(=O)C(N)=O)n(-c2ccnc(C#N)n2)n1]

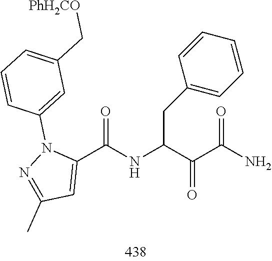

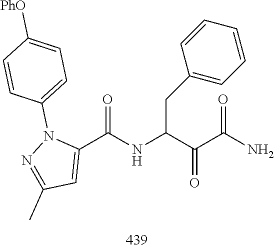 [SMILES: Cc1cc(C(=O)NC(Cc2ccccc2)C(=O)C(N)=O)n(-c2ccc(Oc3ccccc3)cc2)n1]

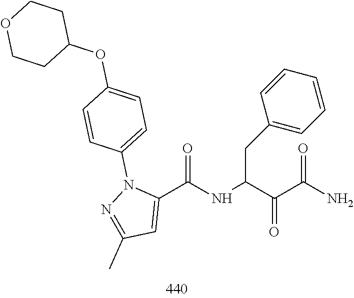 [SMILES: Cc1cc(C(=O)NC(Cc2ccccc2)C(=O)C(N)=O)n(-c2ccc(OC3CCOCC3)cc2)n1]

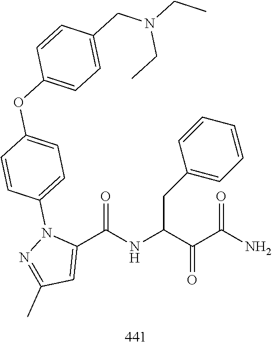 [SMILES: CCN(CC)Cc1ccc(Oc2ccc(-n3nc(C)cc3C(=O)NC(Cc3ccccc3)C(=O)C(N)=O)cc2)cc1]

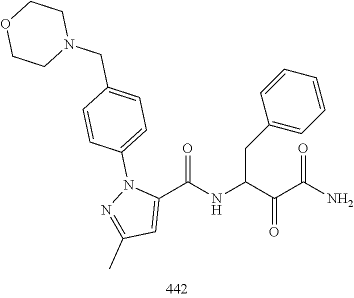 [SMILES: Cc1cc(C(=O)NC(Cc2ccccc2)C(=O)C(N)=O)n(-c2ccc(CN3CCOCC3)cc2)n1]

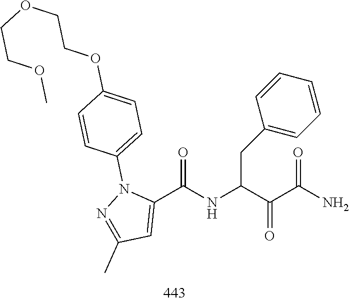 [SMILES: COCCOCCOc1ccc(-n2nc(C)cc2C(=O)NC(Cc2ccccc2)C(=O)C(N)=O)cc1]

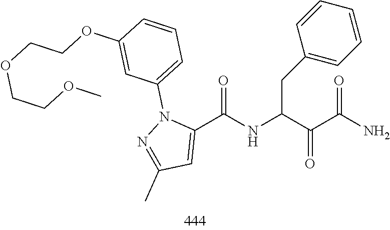 [SMILES: COCCOCCOc1cccc(-n2nc(C)cc2C(=O)NC(Cc2ccccc2)C(=O)C(N)=O)c1]

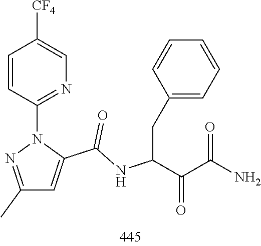

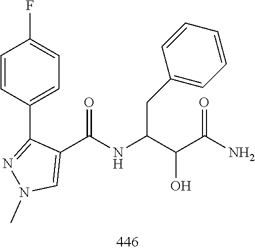 [SMILES: Cn1cc(C(=O)NC(Cc2ccccc2)C(O)C(N)=O)c(-c2ccc(F)cc2)n1]

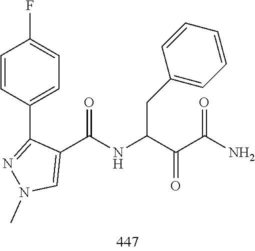 [SMILES: Cn1cc(C(=O)NC(Cc2ccccc2)C(=O)C(N)=O)c(-c2ccc(F)cc2)n1]

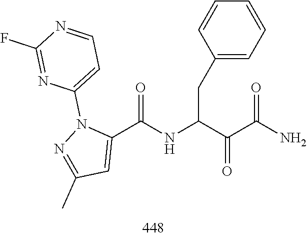 [SMILES: Cc1cc(C(=O)NC(Cc2ccccc2)C(=O)C(N)=O)n(-c2ccnc(F)n2)n1]

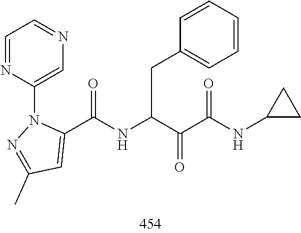 [SMILES: Cc1cc(C(=O)NC(Cc2ccccc2)C(=O)C(=O)NC2CC2)n(-c2cnccn2)n1]

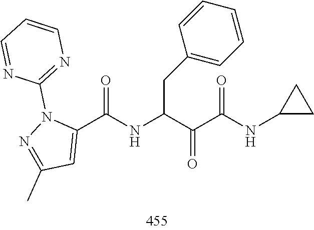 [SMILES: Cc1cc(C(=O)NC(Cc2ccccc2)C(=O)C(=O)NC2CC2)n(-c2ncccn2)n1]

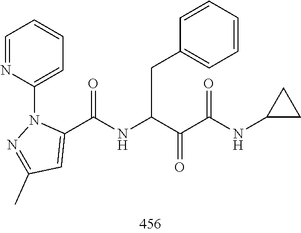 [SMILES: Cc1cc(C(=O)NC(Cc2ccccc2)C(=O)C(=O)NC2CC2)n(-c2ccccn2)n1]

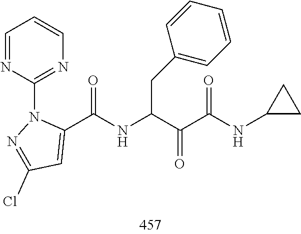 [SMILES: O=C(NC1CC1)C(=O)C(Cc1ccccc1)NC(=O)c1cc(Cl)nn1-c1ncccn1]

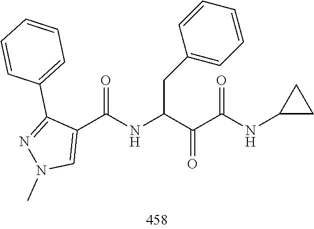 [SMILES: Cn1cc(C(=O)NC(Cc2ccccc2)C(=O)C(=O)NC2CC2)c(-c2ccccc2)n1]

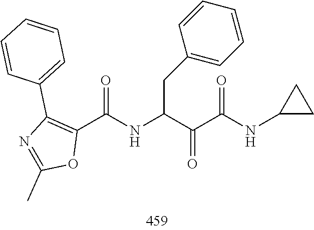 [SMILES: Cc1nc(-c2ccccc2)c(C(=O)NC(Cc2ccccc2)C(=O)C(=O)NC2CC2)o1]

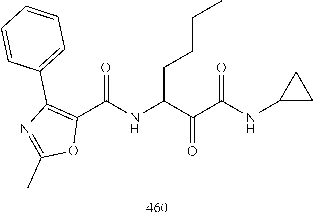 [SMILES: CCCCC(NC(=O)c1oc(C)nc1-c1ccccc1)C(=O)C(=O)NC1CC1]

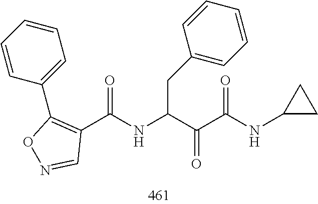 [SMILES: O=C(NC1CC1)C(=O)C(Cc1ccccc1)NC(=O)c1cnoc1-c1ccccc1]

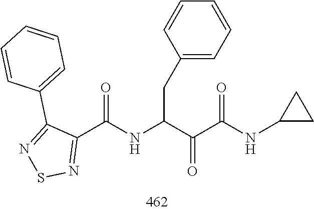 [SMILES: O=C(NC1CC1)C(=O)C(Cc1ccccc1)NC(=O)c1nsnc1-c1ccccc1]

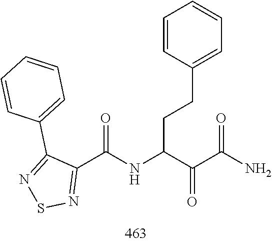 [SMILES: NC(=O)C(=O)C(CCc1ccccc1)NC(=O)c1nsnc1-c1ccccc1]

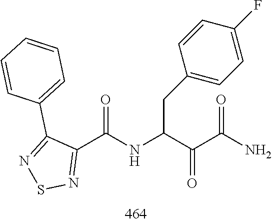 [SMILES: NC(=O)C(=O)C(Cc1ccc(F)cc1)NC(=O)c1nsnc1-c1ccccc1]

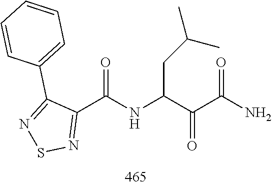 [SMILES: CC(C)CC(NC(=O)c1nsnc1-c1ccccc1)C(=O)C(N)=O]

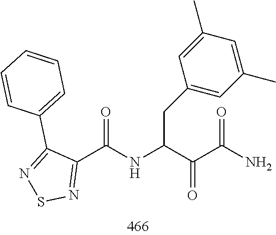 [SMILES: Cc1cc(C)cc(CC(NC(=O)c2nsnc2-c2ccccc2)C(=O)C(N)=O)c1]

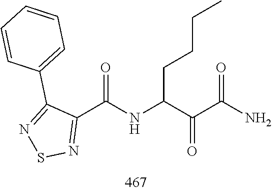 [SMILES: CCCCC(NC(=O)c1nsnc1-c1ccccc1)C(=O)C(N)=O]

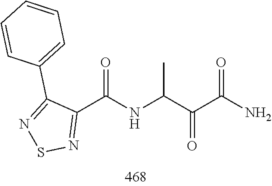 [SMILES: CC(NC(=O)c1nsnc1-c1ccccc1)C(=O)C(N)=O]

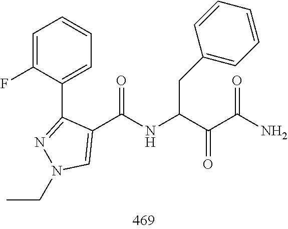 [SMILES: CCn1cc(C(=O)NC(Cc2ccccc2)C(=O)C(N)=O)c(-c2ccccc2F)n1]

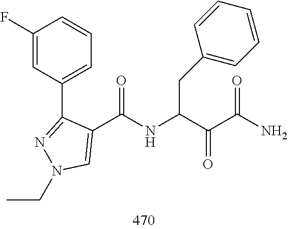 [SMILES: CCn1cc(C(=O)NC(Cc2ccccc2)C(=O)C(N)=O)c(-c2cccc(F)c2)n1]

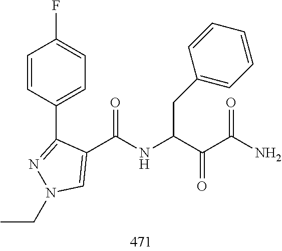 [SMILES: CCn1cc(C(=O)NC(Cc2ccccc2)C(=O)C(N)=O)c(-c2ccc(F)cc2)n1]

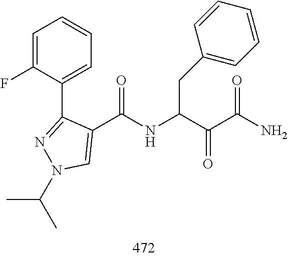 [SMILES: CC(C)n1cc(C(=O)NC(Cc2ccccc2)C(=O)C(N)=O)c(-c2ccccc2F)n1]

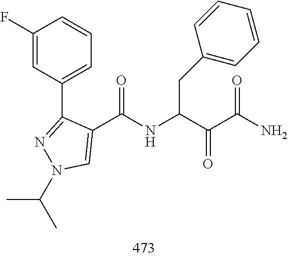 [SMILES: CC(C)n1cc(C(=O)NC(Cc2ccccc2)C(=O)C(N)=O)c(-c2cccc(F)c2)n1]

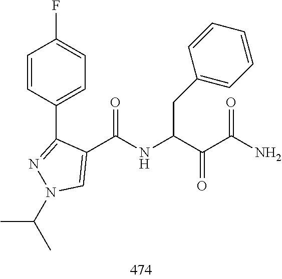 [SMILES: CC(C)n1cc(C(=O)NC(Cc2ccccc2)C(=O)C(N)=O)c(-c2ccc(F)cc2)n1]

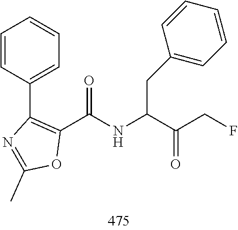 [SMILES: Cc1nc(-c2ccccc2)c(C(=O)NC(Cc2ccccc2)C(=O)CF)o1]

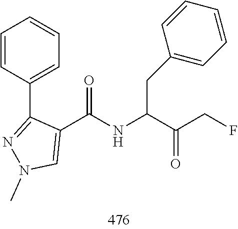 [SMILES: Cn1cc(C(=O)NC(Cc2ccccc2)C(=O)CF)c(-c2ccccc2)n1]

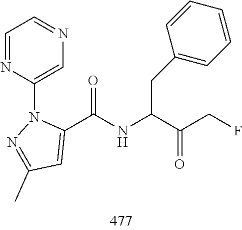 [SMILES: Cc1cc(C(=O)NC(Cc2ccccc2)C(=O)CF)n(-c2cnccn2)n1]

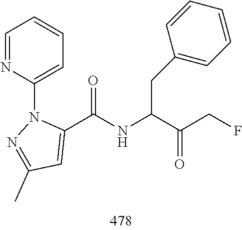 [SMILES: Cc1cc(C(=O)NC(Cc2ccccc2)C(=O)CF)n(-c2ccccn2)n1]

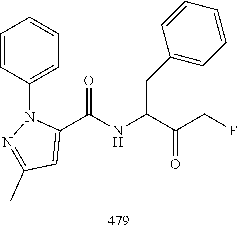 [SMILES: Cc1cc(C(=O)NC(Cc2ccccc2)C(=O)CF)n(-c2ccccc2)n1]

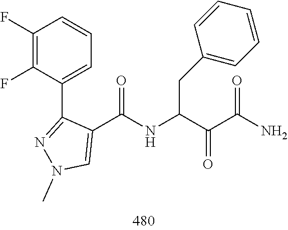 [SMILES: Cn1cc(C(=O)NC(Cc2ccccc2)C(=O)C(N)=O)c(-c2cccc(F)c2F)n1]

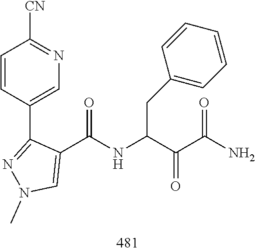 [SMILES: Cn1cc(C(=O)NC(Cc2ccccc2)C(=O)C(N)=O)c(-c2ccc(C#N)nc2)n1]

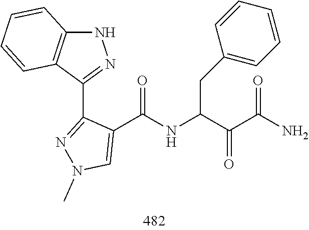 [SMILES: Cn1cc(C(=O)NC(Cc2ccccc2)C(=O)C(N)=O)c(-c2n[nH]c3ccccc23)n1]

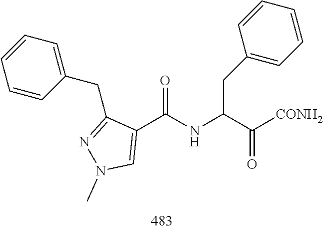 [SMILES: Cn1cc(C(=O)NC(Cc2ccccc2)C(=O)C(N)=O)c(Cc2ccccc2)n1]

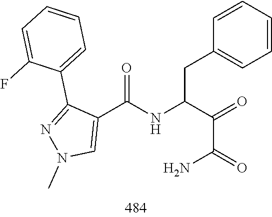 [SMILES: Cn1cc(C(=O)NC(Cc2ccccc2)C(=O)C(N)=O)c(-c2ccccc2F)n1]

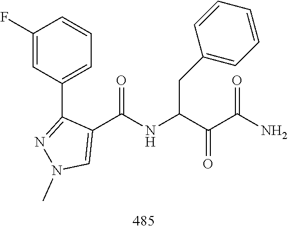 [SMILES: Cn1cc(C(=O)NC(Cc2ccccc2)C(=O)C(N)=O)c(-c2cccc(F)c2)n1]

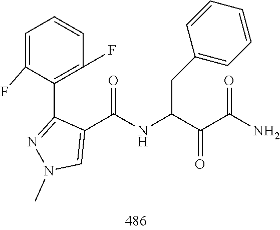 [SMILES: Cn1cc(C(=O)NC(Cc2ccccc2)C(=O)C(N)=O)c(-c2c(F)cccc2F)n1]

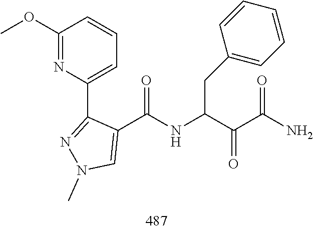 [SMILES: COc1cccc(-c2nn(C)cc2C(=O)NC(Cc2ccccc2)C(=O)C(N)=O)n1]

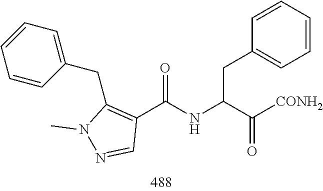 [SMILES: Cn1ncc(C(=O)NC(Cc2ccccc2)C(=O)C(N)=O)c1Cc1ccccc1]

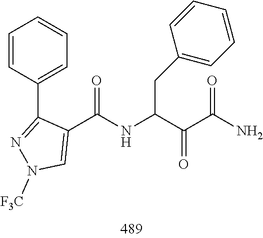 [SMILES: NC(=O)C(=O)C(Cc1ccccc1)NC(=O)c1cn(C(F)(F)F)nc1-c1ccccc1]

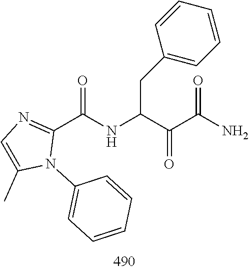 [SMILES: Cc1cnc(C(=O)NC(Cc2ccccc2)C(=O)C(N)=O)n1-c1ccccc1]

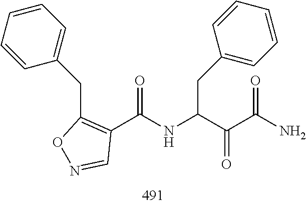 [SMILES: NC(=O)C(=O)C(Cc1ccccc1)NC(=O)c1cnoc1Cc1ccccc1]

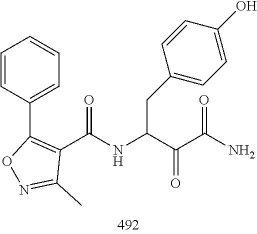 [SMILES: Cc1noc(-c2ccccc2)c1C(=O)NC(Cc1ccc(O)cc1)C(=O)C(N)=O]

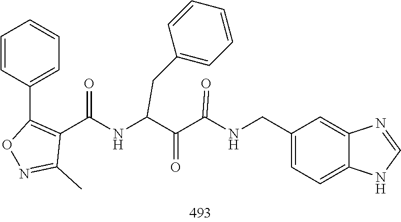 [SMILES: Cc1noc(-c2ccccc2)c1C(=O)NC(Cc1ccccc1)C(=O)C(=O)NCc1ccc2[nH]cnc2c1]

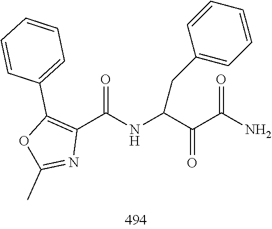 [SMILES: Cc1nc(C(=O)NC(Cc2ccccc2)C(=O)C(N)=O)c(-c2ccccc2)o1]

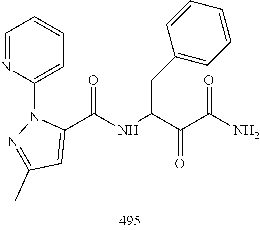 [SMILES: Cc1cc(C(=O)NC(Cc2ccccc2)C(=O)C(N)=O)n(-c2ccccn2)n1]

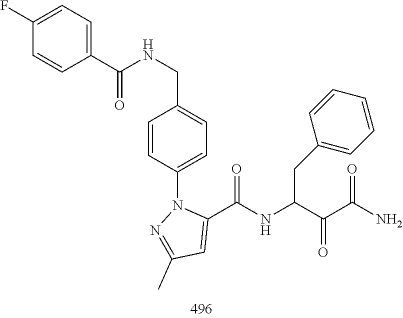 [SMILES: Cc1cc(C(=O)NC(Cc2ccccc2)C(=O)C(N)=O)n(-c2ccc(CNC(=O)c3ccc(F)cc3)cc2)n1]

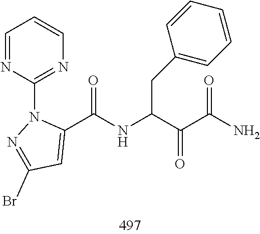 [SMILES: NC(=O)C(=O)C(Cc1ccccc1)NC(=O)c1cc(Br)nn1-c1ncccn1]

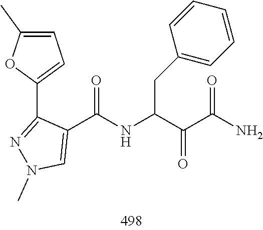 [SMILES: Cc1ccc(-c2nn(C)cc2C(=O)NC(Cc2ccccc2)C(=O)C(N)=O)o1]

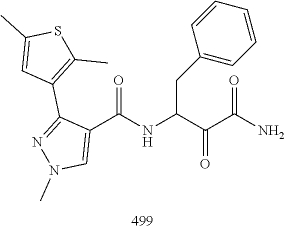 [SMILES: Cc1cc(-c2nn(C)cc2C(=O)NC(Cc2ccccc2)C(=O)C(N)=O)c(C)s1]

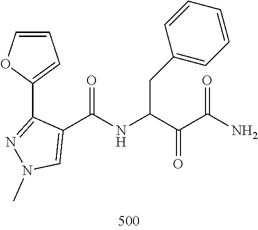 [SMILES: Cn1cc(C(=O)NC(Cc2ccccc2)C(=O)C(N)=O)c(-c2ccco2)n1]

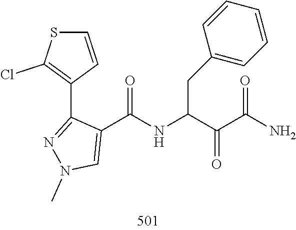 [SMILES: Cn1cc(C(=O)NC(Cc2ccccc2)C(=O)C(N)=O)c(-c2ccsc2Cl)n1]

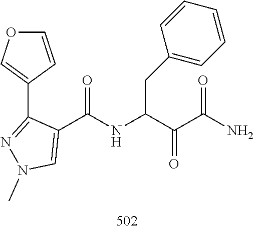 [SMILES: Cn1cc(C(=O)NC(Cc2ccccc2)C(=O)C(N)=O)c(-c2ccoc2)n1]

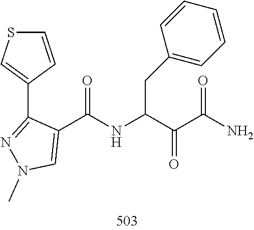 [SMILES: Cn1cc(C(=O)NC(Cc2ccccc2)C(=O)C(N)=O)c(-c2ccsc2)n1]

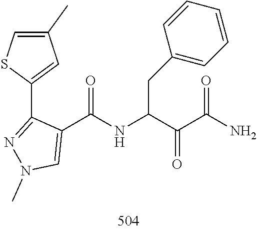 [SMILES: Cc1csc(-c2nn(C)cc2C(=O)NC(Cc2ccccc2)C(=O)C(N)=O)c1]

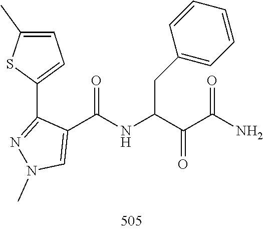 [SMILES: Cc1ccc(-c2nn(C)cc2C(=O)NC(Cc2ccccc2)C(=O)C(N)=O)s1]

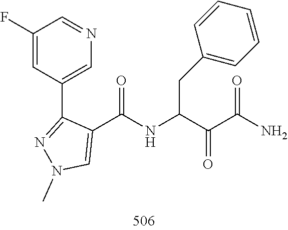 [SMILES: Cn1cc(C(=O)NC(Cc2ccccc2)C(=O)C(N)=O)c(-c2cncc(F)c2)n1]

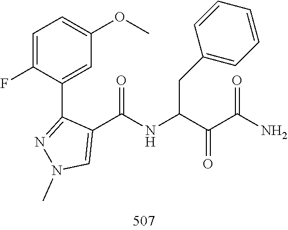 [SMILES: COc1ccc(F)c(-c2nn(C)cc2C(=O)NC(Cc2ccccc2)C(=O)C(N)=O)c1]

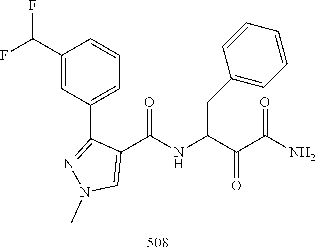 [SMILES: Cn1cc(C(=O)NC(Cc2ccccc2)C(=O)C(N)=O)c(-c2cccc(C(F)F)c2)n1]

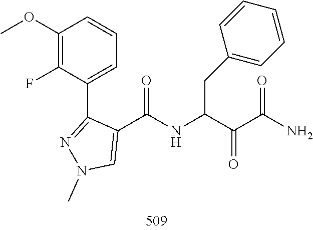 [SMILES: COc1cccc(-c2nn(C)cc2C(=O)NC(Cc2ccccc2)C(=O)C(N)=O)c1F]

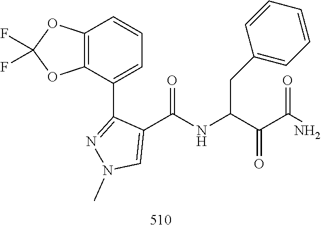 [SMILES: Cn1cc(C(=O)NC(Cc2ccccc2)C(=O)C(N)=O)c(-c2cccc3c2OC(F)(F)O3)n1]

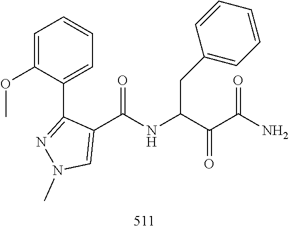 [SMILES: COc1ccccc1-c1nn(C)cc1C(=O)NC(Cc1ccccc1)C(=O)C(N)=O]

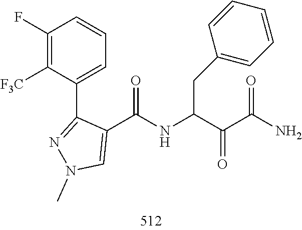 [SMILES: Cn1cc(C(=O)NC(Cc2ccccc2)C(=O)C(N)=O)c(-c2cccc(F)c2C(F)(F)F)n1]

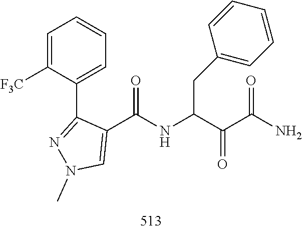 [SMILES: Cn1cc(C(=O)NC(Cc2ccccc2)C(=O)C(N)=O)c(-c2ccccc2C(F)(F)F)n1]

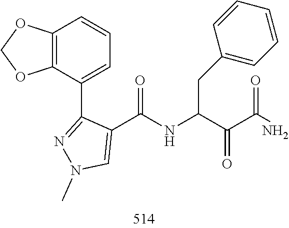 [SMILES: Cn1cc(C(=O)NC(Cc2ccccc2)C(=O)C(N)=O)c(-c2cccc3c2OCO3)n1]

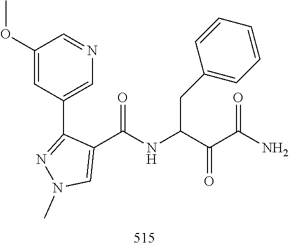 [SMILES: COc1cncc(-c2nn(C)cc2C(=O)NC(Cc2ccccc2)C(=O)C(N)=O)c1]

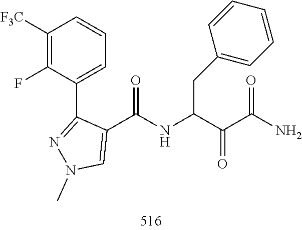 [SMILES: Cn1cc(C(=O)NC(Cc2ccccc2)C(=O)C(N)=O)c(-c2cccc(C(F)(F)F)c2F)n1]

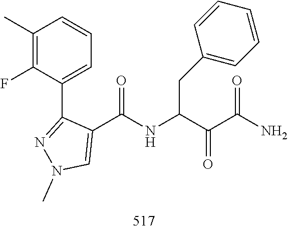 [SMILES: Cc1cccc(-c2nn(C)cc2C(=O)NC(Cc2ccccc2)C(=O)C(N)=O)c1F]

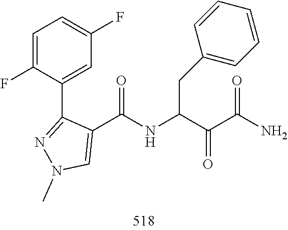 [SMILES: Cn1cc(C(=O)NC(Cc2ccccc2)C(=O)C(N)=O)c(-c2cc(F)ccc2F)n1]

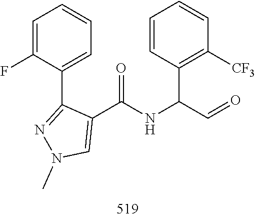 [SMILES: Cn1cc(C(=O)NC(C=O)c2ccccc2C(F)(F)F)c(-c2ccccc2F)n1]

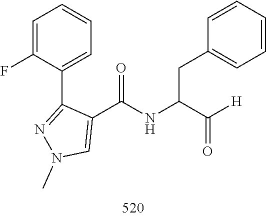 [SMILES: Cn1cc(C(=O)NC(C=O)Cc2ccccc2)c(-c2ccccc2F)n1]

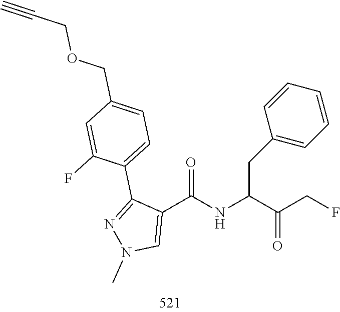 [SMILES: C#CCOCc1ccc(-c2nn(C)cc2C(=O)NC(Cc2ccccc2)C(=O)CF)c(F)c1]

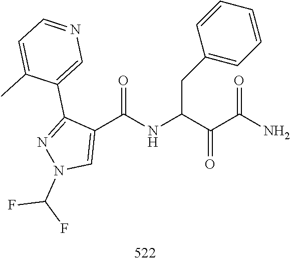 [SMILES: Cc1ccncc1-c1nn(C(F)F)cc1C(=O)NC(Cc1ccccc1)C(=O)C(N)=O]

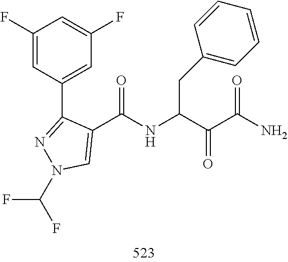 [SMILES: NC(=O)C(=O)C(Cc1ccccc1)NC(=O)c1cn(C(F)F)nc1-c1cc(F)cc(F)c1]

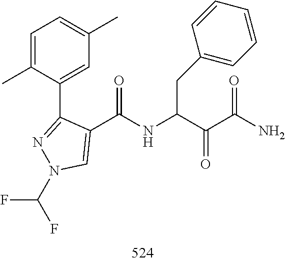 [SMILES: Cc1ccc(C)c(-c2nn(C(F)F)cc2C(=O)NC(Cc2ccccc2)C(=O)C(N)=O)c1]

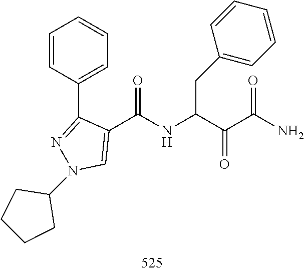 [SMILES: NC(=O)C(=O)C(Cc1ccccc1)NC(=O)c1cn(C2CCCC2)nc1-c1ccccc1]

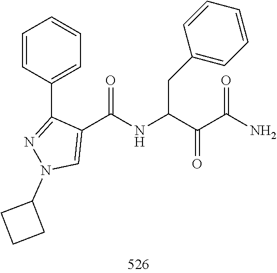 [SMILES: NC(=O)C(=O)C(Cc1ccccc1)NC(=O)c1cn(C2CCC2)nc1-c1ccccc1]

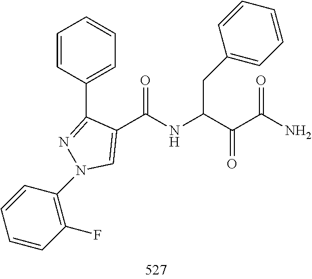 [SMILES: NC(=O)C(=O)C(Cc1ccccc1)NC(=O)c1cn(-c2ccccc2F)nc1-c1ccccc1]

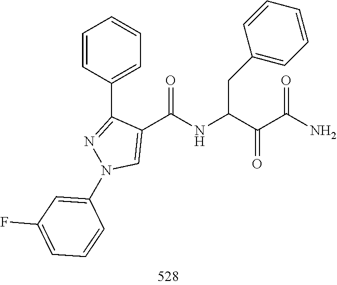 [SMILES: NC(=O)C(=O)C(Cc1ccccc1)NC(=O)c1cn(-c2cccc(F)c2)nc1-c1ccccc1]

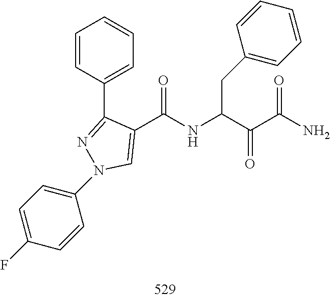 [SMILES: NC(=O)C(=O)C(Cc1ccccc1)NC(=O)c1cn(-c2ccc(F)cc2)nc1-c1ccccc1]

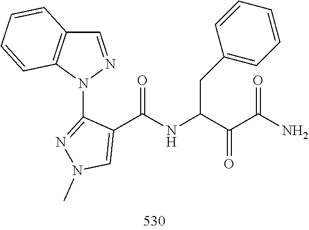 [SMILES: Cn1cc(C(=O)NC(Cc2ccccc2)C(=O)C(N)=O)c(-n2ncc3ccccc32)n1]

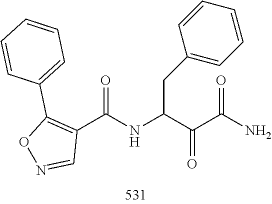 [SMILES: NC(=O)C(=O)C(Cc1ccccc1)NC(=O)c1cnoc1-c1ccccc1]

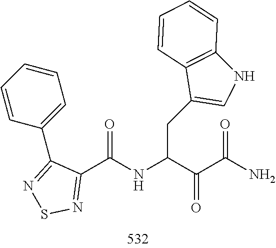 [SMILES: NC(=O)C(=O)C(Cc1c[nH]c2ccccc12)NC(=O)c1nsnc1-c1ccccc1]

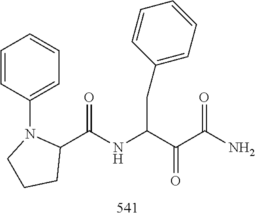 [SMILES: NC(=O)C(=O)C(Cc1ccccc1)NC(=O)C1CCCN1c1ccccc1]

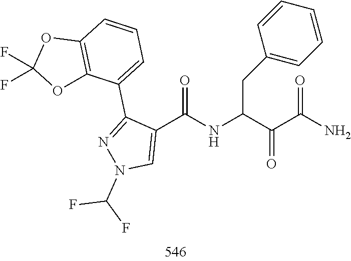 [SMILES: NC(=O)C(=O)C(Cc1ccccc1)NC(=O)c1cn(C(F)F)nc1-c1cccc2c1OC(F)(F)O2]

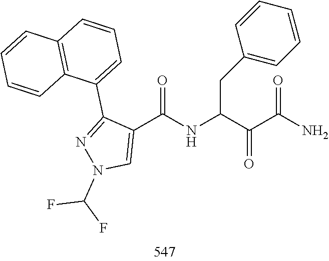 [SMILES: NC(=O)C(=O)C(Cc1ccccc1)NC(=O)c1cn(C(F)F)nc1-c1cccc2ccccc12]

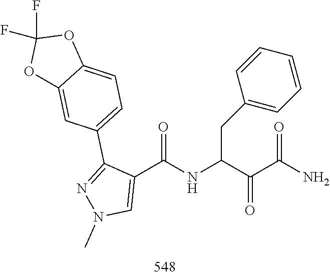 [SMILES: Cn1cc(C(=O)NC(Cc2ccccc2)C(=O)C(N)=O)c(-c2ccc3c(c2)OC(F)(F)O3)n1]

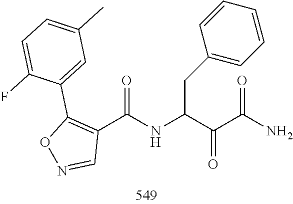 [SMILES: Cc1ccc(F)c(-c2oncc2C(=O)NC(Cc2ccccc2)C(=O)C(N)=O)c1]

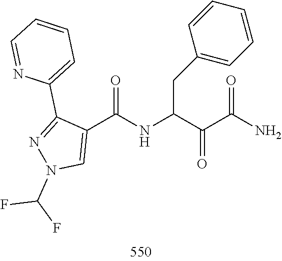 [SMILES: NC(=O)C(=O)C(Cc1ccccc1)NC(=O)c1cn(C(F)F)nc1-c1ccccn1]

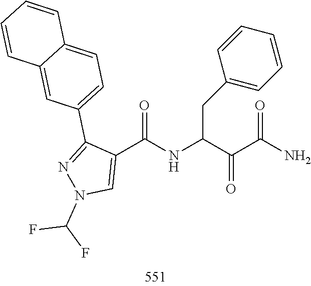 [SMILES: NC(=O)C(=O)C(Cc1ccccc1)NC(=O)c1cn(C(F)F)nc1-c1ccc2ccccc2c1]

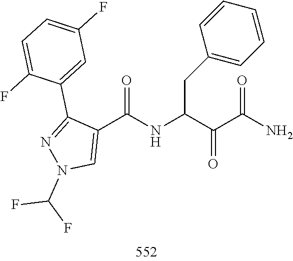 [SMILES: NC(=O)C(=O)C(Cc1ccccc1)NC(=O)c1cn(C(F)F)nc1-c1cc(F)ccc1F]

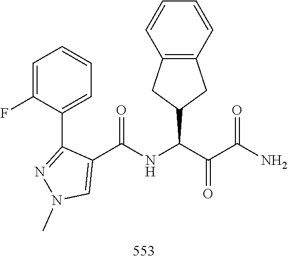 [SMILES: Cn1cc(C(=O)N[C@H](C(=O)C(N)=O)C2Cc3ccccc3C2)c(-c2ccccc2F)n1]

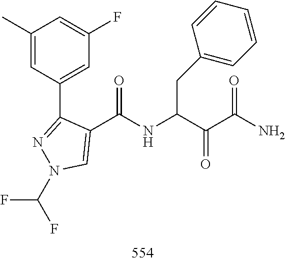 [SMILES: Cc1cc(F)cc(-c2nn(C(F)F)cc2C(=O)NC(Cc2ccccc2)C(=O)C(N)=O)c1]

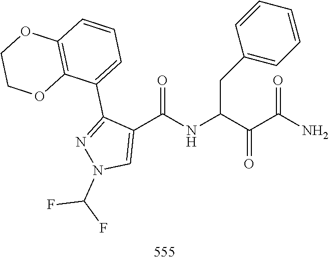 [SMILES: NC(=O)C(=O)C(Cc1ccccc1)NC(=O)c1cn(C(F)F)nc1-c1cccc2c1OCCO2]

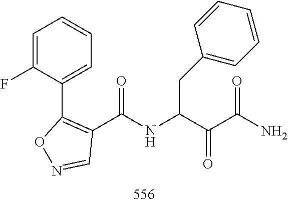 [SMILES: NC(=O)C(=O)C(Cc1ccccc1)NC(=O)c1cnoc1-c1ccccc1F]

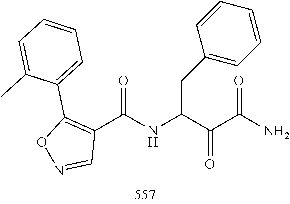 [SMILES: Cc1ccccc1-c1oncc1C(=O)NC(Cc1ccccc1)C(=O)C(N)=O]

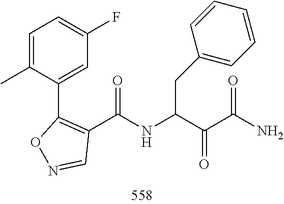 [SMILES: Cc1ccc(F)cc1-c1oncc1C(=O)NC(Cc1ccccc1)C(=O)C(N)=O]

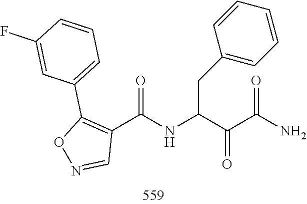 [SMILES: NC(=O)C(=O)C(Cc1ccccc1)NC(=O)c1cnoc1-c1cccc(F)c1]

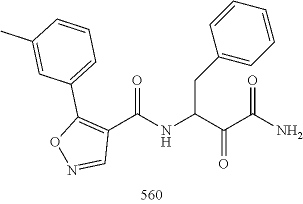 [SMILES: Cc1cccc(-c2oncc2C(=O)NC(Cc2ccccc2)C(=O)C(N)=O)c1]

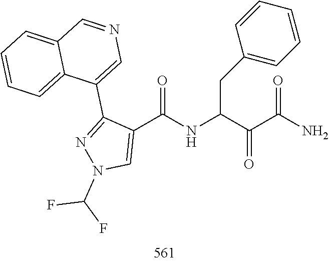 [SMILES: NC(=O)C(=O)C(Cc1ccccc1)NC(=O)c1cn(C(F)F)nc1-c1cncc2ccccc12]

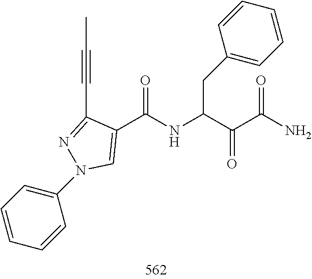 [SMILES: CC#Cc1nn(-c2ccccc2)cc1C(=O)NC(Cc1ccccc1)C(=O)C(N)=O]

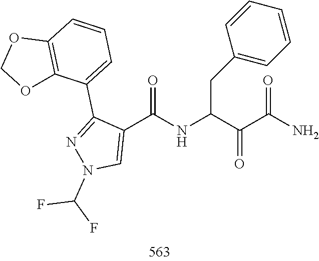 [SMILES: NC(=O)C(=O)C(Cc1ccccc1)NC(=O)c1cn(C(F)F)nc1-c1cccc2c1OCO2]

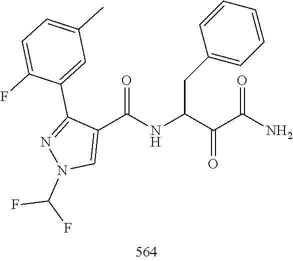 [SMILES: Cc1ccc(F)c(-c2nn(C(F)F)cc2C(=O)NC(Cc2ccccc2)C(=O)C(N)=O)c1]

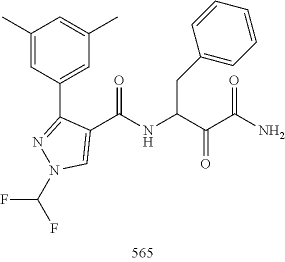 [SMILES: Cc1cc(C)cc(-c2nn(C(F)F)cc2C(=O)NC(Cc2ccccc2)C(=O)C(N)=O)c1]

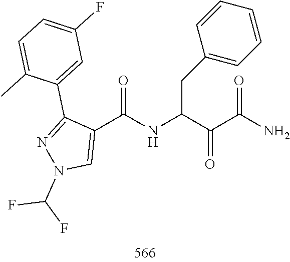 [SMILES: Cc1ccc(F)cc1-c1nn(C(F)F)cc1C(=O)NC(Cc1ccccc1)C(=O)C(N)=O]

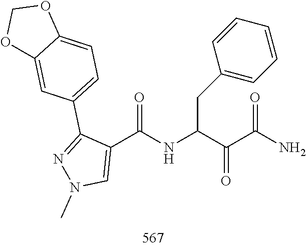 [SMILES: Cn1cc(C(=O)NC(Cc2ccccc2)C(=O)C(N)=O)c(-c2ccc3c(c2)OCO3)n1]

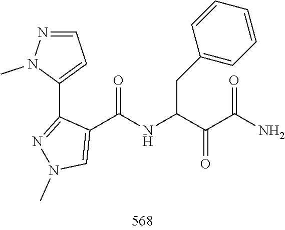 [SMILES: Cn1cc(C(=O)NC(Cc2ccccc2)C(=O)C(N)=O)c(-c2ccnn2C)n1]

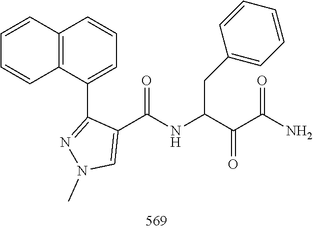 [SMILES: Cn1cc(C(=O)NC(Cc2ccccc2)C(=O)C(N)=O)c(-c2cccc3ccccc23)n1]

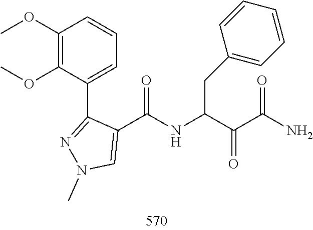 [SMILES: COc1cccc(-c2nn(C)cc2C(=O)NC(Cc2ccccc2)C(=O)C(N)=O)c1OC]

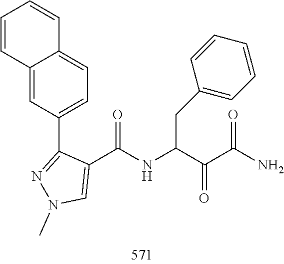 [SMILES: Cn1cc(C(=O)NC(Cc2ccccc2)C(=O)C(N)=O)c(-c2ccc3ccccc3c2)n1]

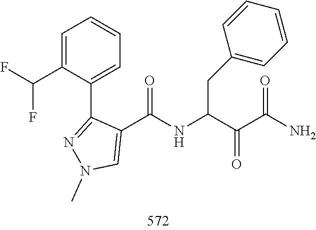 [SMILES: Cn1cc(C(=O)NC(Cc2ccccc2)C(=O)C(N)=O)c(-c2ccccc2C(F)F)n1]

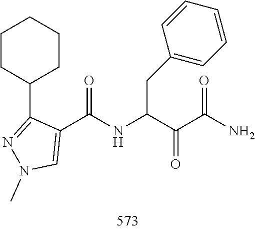 [SMILES: Cn1cc(C(=O)NC(Cc2ccccc2)C(=O)C(N)=O)c(C2CCCCC2)n1]

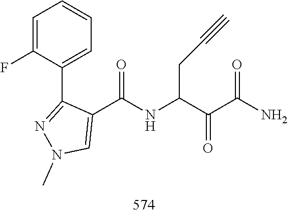 [SMILES: C#CCC(NC(=O)c1cn(C)nc1-c1ccccc1F)C(=O)C(N)=O]

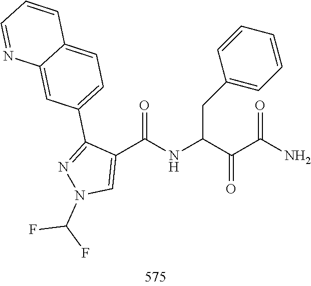 [SMILES: NC(=O)C(=O)C(Cc1ccccc1)NC(=O)c1cn(C(F)F)nc1-c1ccc2cccnc2c1]

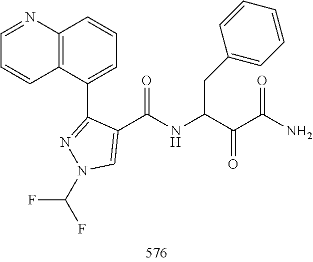 [SMILES: NC(=O)C(=O)C(Cc1ccccc1)NC(=O)c1cn(C(F)F)nc1-c1cccc2ncccc12]

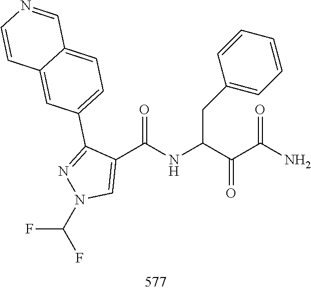 [SMILES: NC(=O)C(=O)C(Cc1ccccc1)NC(=O)c1cn(C(F)F)nc1-c1ccc2cnccc2c1]

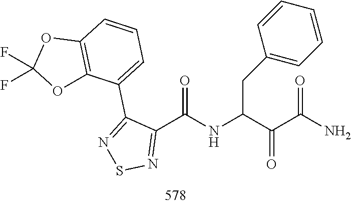 [SMILES: NC(=O)C(=O)C(Cc1ccccc1)NC(=O)c1nsnc1-c1cccc2c1OC(F)(F)O2]

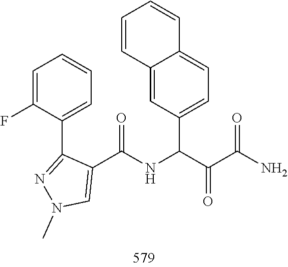 [SMILES: Cn1cc(C(=O)NC(C(=O)C(N)=O)c2ccc3ccccc3c2)c(-c2ccccc2F)n1]

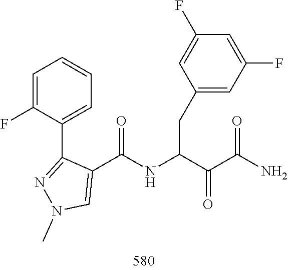 [SMILES: Cn1cc(C(=O)NC(Cc2cc(F)cc(F)c2)C(=O)C(N)=O)c(-c2ccccc2F)n1]

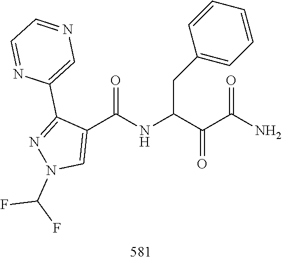 [SMILES: NC(=O)C(=O)C(Cc1ccccc1)NC(=O)c1cn(C(F)F)nc1-c1cnccn1]

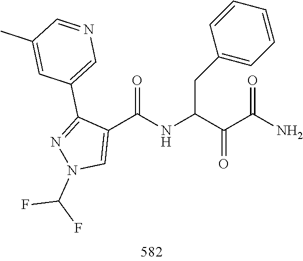 [SMILES: Cc1cncc(-c2nn(C(F)F)cc2C(=O)NC(Cc2ccccc2)C(=O)C(N)=O)c1]

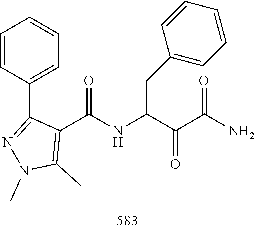 [SMILES: Cc1c(C(=O)NC(Cc2ccccc2)C(=O)C(N)=O)c(-c2ccccc2)nn1C]

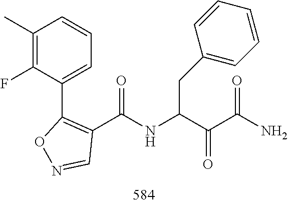 [SMILES: Cc1cccc(-c2oncc2C(=O)NC(Cc2ccccc2)C(=O)C(N)=O)c1F]

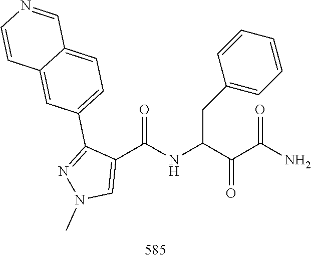 [SMILES: Cn1cc(C(=O)NC(Cc2ccccc2)C(=O)C(N)=O)c(-c2ccc3cnccc3c2)n1]

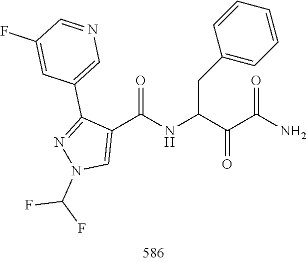 [SMILES: NC(=O)C(=O)C(Cc1ccccc1)NC(=O)c1cn(C(F)F)nc1-c1cncc(F)c1]

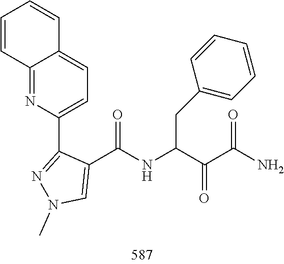 [SMILES: Cn1cc(C(=O)NC(Cc2ccccc2)C(=O)C(N)=O)c(-c2ccc3ccccc3n2)n1]

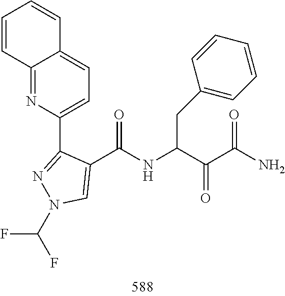 [SMILES: NC(=O)C(=O)C(Cc1ccccc1)NC(=O)c1cn(C(F)F)nc1-c1ccc2ccccc2n1]

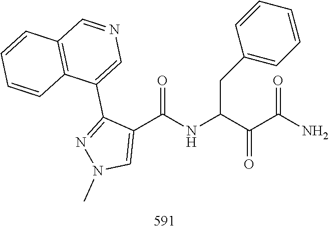 [SMILES: Cn1cc(C(=O)NC(Cc2ccccc2)C(=O)C(N)=O)c(-c2cncc3ccccc23)n1]

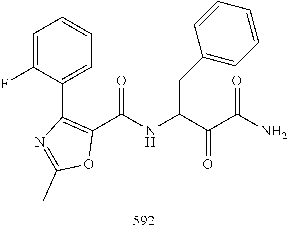 [SMILES: Cc1nc(-c2ccccc2F)c(C(=O)NC(Cc2ccccc2)C(=O)C(N)=O)o1]

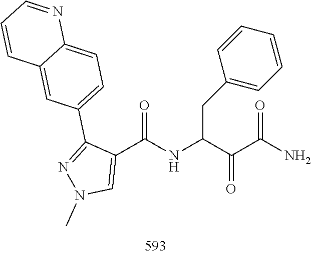 [SMILES: Cn1cc(C(=O)NC(Cc2ccccc2)C(=O)C(N)=O)c(-c2ccc3ncccc3c2)n1]

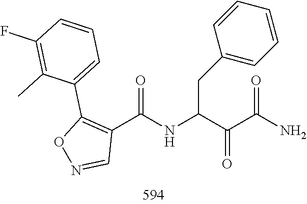 [SMILES: Cc1c(F)cccc1-c1oncc1C(=O)NC(Cc1ccccc1)C(=O)C(N)=O]

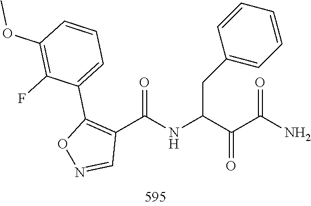 [SMILES: COc1cccc(-c2oncc2C(=O)NC(Cc2ccccc2)C(=O)C(N)=O)c1F]

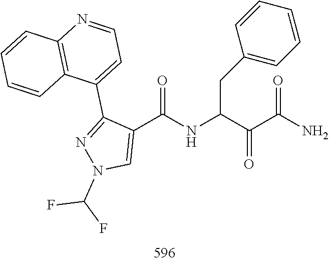 [SMILES: NC(=O)C(=O)C(Cc1ccccc1)NC(=O)c1cn(C(F)F)nc1-c1ccnc2ccccc12]

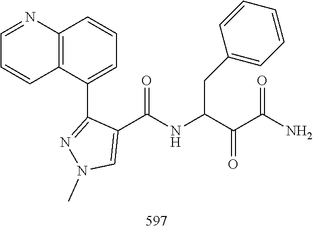 [SMILES: Cn1cc(C(=O)NC(Cc2ccccc2)C(=O)C(N)=O)c(-c2cccc3ncccc23)n1]

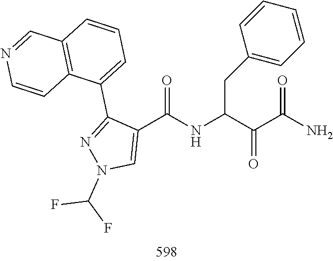 [SMILES: NC(=O)C(=O)C(Cc1ccccc1)NC(=O)c1cn(C(F)F)nc1-c1cccc2cnccc12]

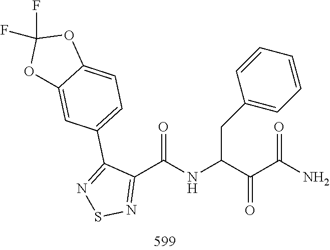 [SMILES: NC(=O)C(=O)C(Cc1ccccc1)NC(=O)c1nsnc1-c1ccc2c(c1)OC(F)(F)O2]

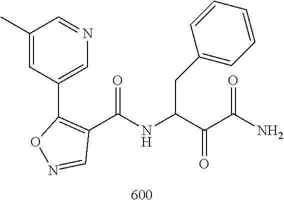 [SMILES: Cc1cncc(-c2oncc2C(=O)NC(Cc2ccccc2)C(=O)C(N)=O)c1]

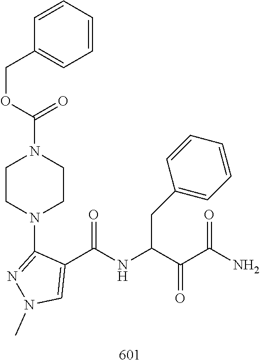 [SMILES: Cn1cc(C(=O)NC(Cc2ccccc2)C(=O)C(N)=O)c(N2CCN(C(=O)OCc3ccccc3)CC2)n1]

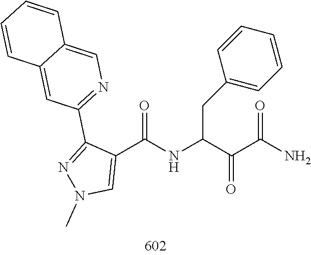 [SMILES: Cn1cc(C(=O)NC(Cc2ccccc2)C(=O)C(N)=O)c(-c2cc3ccccc3cn2)n1]

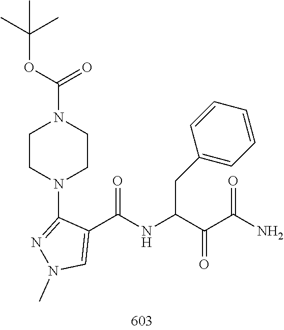 [SMILES: Cn1cc(C(=O)NC(Cc2ccccc2)C(=O)C(N)=O)c(N2CCN(C(=O)OC(C)(C)C)CC2)n1]

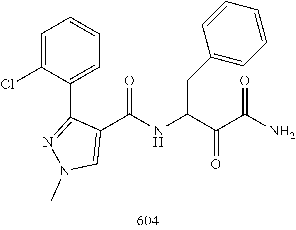 [SMILES: Cn1cc(C(=O)NC(Cc2ccccc2)C(=O)C(N)=O)c(-c2ccccc2Cl)n1]

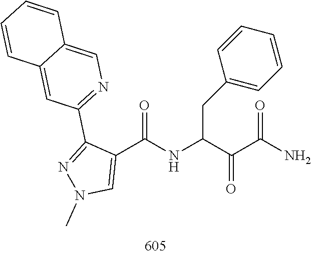 [SMILES: Cn1cc(C(=O)NC(Cc2ccccc2)C(=O)C(N)=O)c(-c2cc3ccccc3cn2)n1]

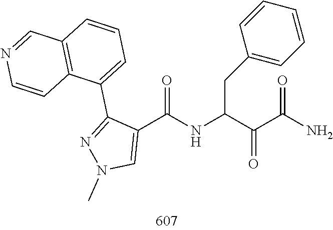 [SMILES: Cn1cc(C(=O)NC(Cc2ccccc2)C(=O)C(N)=O)c(-c2cccc3cnccc23)n1]

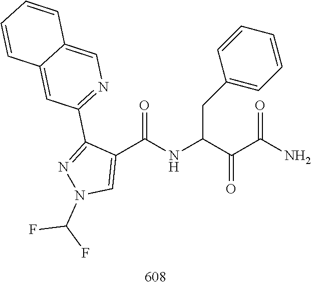 [SMILES: NC(=O)C(=O)C(Cc1ccccc1)NC(=O)c1cn(C(F)F)nc1-c1cc2ccccc2cn1]

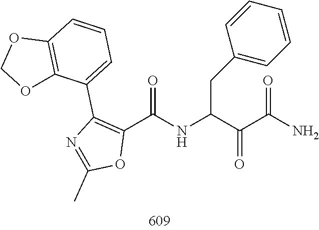 [SMILES: Cc1nc(-c2cccc3c2OCO3)c(C(=O)NC(Cc2ccccc2)C(=O)C(N)=O)o1]

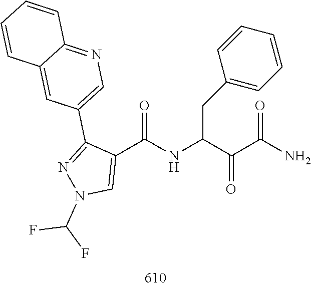 [SMILES: NC(=O)C(=O)C(Cc1ccccc1)NC(=O)c1cn(C(F)F)nc1-c1cnc2ccccc2c1]

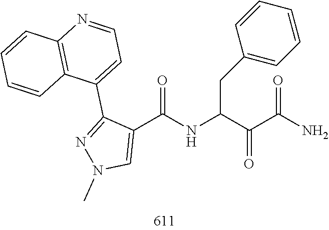 [SMILES: Cn1cc(C(=O)NC(Cc2ccccc2)C(=O)C(N)=O)c(-c2ccnc3ccccc23)n1]

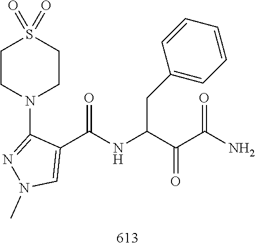 [SMILES: Cn1cc(C(=O)NC(Cc2ccccc2)C(=O)C(N)=O)c(N2CCS(=O)(=O)CC2)n1]

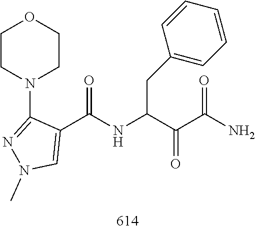 [SMILES: Cn1cc(C(=O)NC(Cc2ccccc2)C(=O)C(N)=O)c(N2CCOCC2)n1]

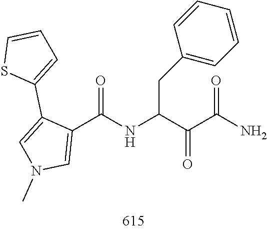 [SMILES: Cn1cc(C(=O)NC(Cc2ccccc2)C(=O)C(N)=O)c(-c2cccs2)c1]

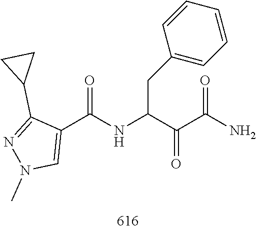 [SMILES: Cn1cc(C(=O)NC(Cc2ccccc2)C(=O)C(N)=O)c(C2CC2)n1]

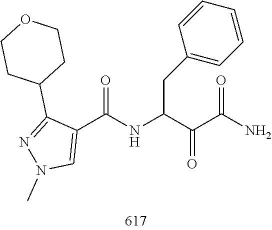 [SMILES: Cn1cc(C(=O)NC(Cc2ccccc2)C(=O)C(N)=O)c(C2CCOCC2)n1]

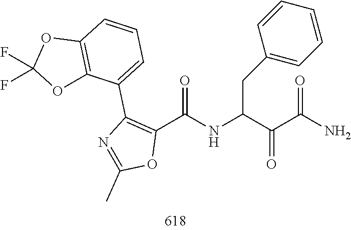 [SMILES: Cc1nc(-c2cccc3c2OC(F)(F)O3)c(C(=O)NC(Cc2ccccc2)C(=O)C(N)=O)o1]

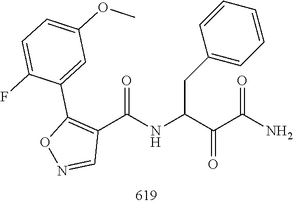 [SMILES: COc1ccc(F)c(-c2oncc2C(=O)NC(Cc2ccccc2)C(=O)C(N)=O)c1]

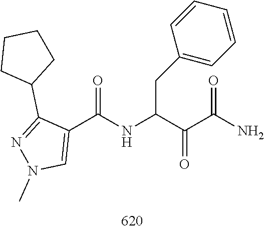 [SMILES: Cn1cc(C(=O)NC(Cc2ccccc2)C(=O)C(N)=O)c(C2CCCC2)n1]

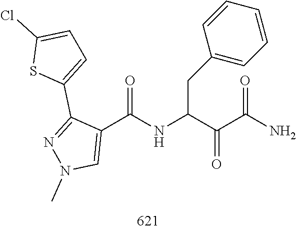 [SMILES: Cn1cc(C(=O)NC(Cc2ccccc2)C(=O)C(N)=O)c(-c2ccc(Cl)s2)n1]

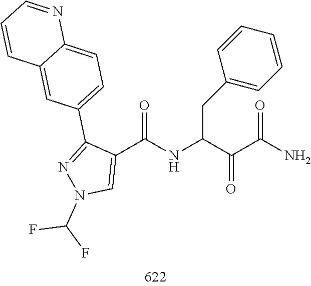 [SMILES: NC(=O)C(=O)C(Cc1ccccc1)NC(=O)c1cn(C(F)F)nc1-c1ccc2ncccc2c1]

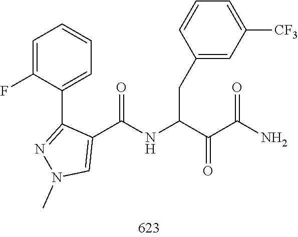 [SMILES: Cn1cc(C(=O)NC(Cc2cccc(C(F)(F)F)c2)C(=O)C(N)=O)c(-c2ccccc2F)n1]

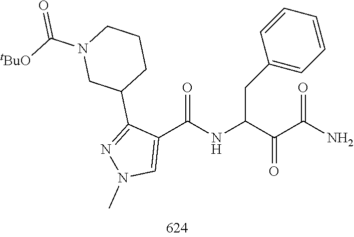 [SMILES: Cn1cc(C(=O)NC(Cc2ccccc2)C(=O)C(N)=O)c(C2CCCN(C(=O)OC(C)(C)C)C2)n1]

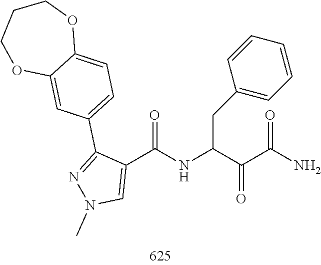 [SMILES: Cn1cc(C(=O)NC(Cc2ccccc2)C(=O)C(N)=O)c(-c2ccc3c(c2)OCCCO3)n1]

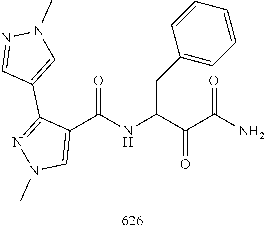 [SMILES: Cn1cc(-c2nn(C)cc2C(=O)NC(Cc2ccccc2)C(=O)C(N)=O)cn1]

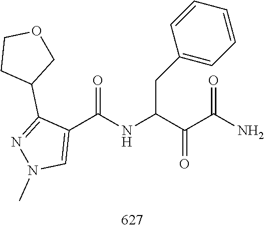 [SMILES: Cn1cc(C(=O)NC(Cc2ccccc2)C(=O)C(N)=O)c(C2CCOC2)n1]

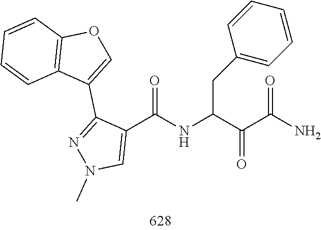 [SMILES: Cn1cc(C(=O)NC(Cc2ccccc2)C(=O)C(N)=O)c(-c2coc3ccccc23)n1]

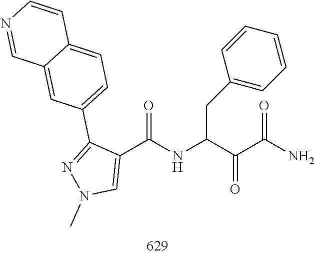 [SMILES: Cn1cc(C(=O)NC(Cc2ccccc2)C(=O)C(N)=O)c(-c2ccc3ccncc3c2)n1]

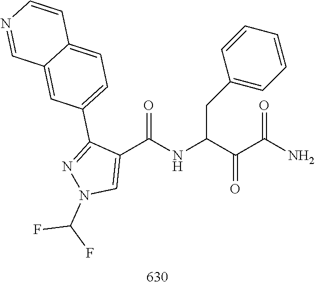 [SMILES: NC(=O)C(=O)C(Cc1ccccc1)NC(=O)c1cn(C(F)F)nc1-c1ccc2ccncc2c1]

TABLE 1b

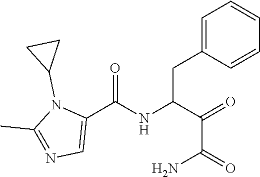 [SMILES: Cc1ncc(C(=O)NC(Cc2ccccc2)C(=O)C(N)=O)n1C1CC1]

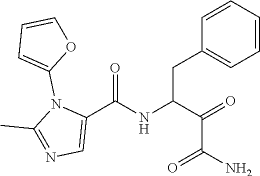 [SMILES: Cc1ncc(C(=O)NC(Cc2ccccc2)C(=O)C(N)=O)n1-c1ccco1]

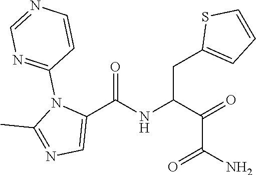 [SMILES: Cc1ncc(C(=O)NC(Cc2cccs2)C(=O)C(N)=O)n1-c1ccncn1]

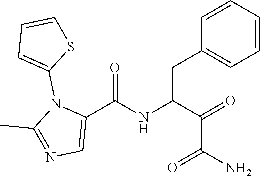 [SMILES: Cc1ncc(C(=O)NC(Cc2ccccc2)C(=O)C(N)=O)n1-c1cccs1]

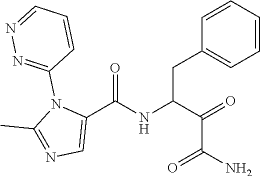 [SMILES: Cc1ncc(C(=O)NC(Cc2ccccc2)C(=O)C(N)=O)n1-c1cccnn1]

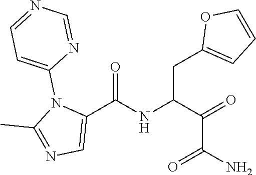 [SMILES: Cc1ncc(C(=O)NC(Cc2ccco2)C(=O)C(N)=O)n1-c1ccncn1]

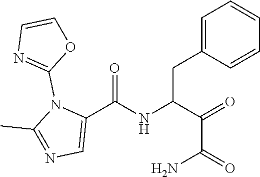 [SMILES: Cc1ncc(C(=O)NC(Cc2ccccc2)C(=O)C(N)=O)n1-c1ncco1]

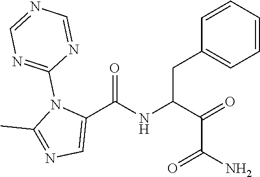 [SMILES: Cc1ncc(C(=O)NC(Cc2ccccc2)C(=O)C(N)=O)n1-c1ncncn1]

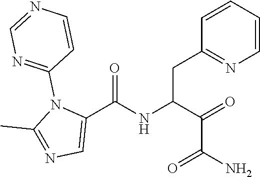 [SMILES: Cc1ncc(C(=O)NC(Cc2ccccn2)C(=O)C(N)=O)n1-c1ccncn1]

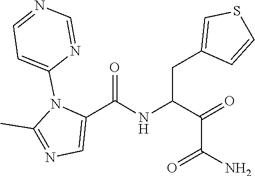 [SMILES: Cc1ncc(C(=O)NC(Cc2ccsc2)C(=O)C(N)=O)n1-c1ccncn1]

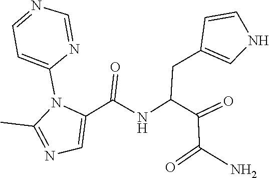 [SMILES: Cc1ncc(C(=O)NC(Cc2cc[nH]c2)C(=O)C(N)=O)n1-c1ccncn1]

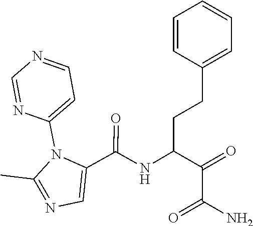 [SMILES: Cc1ncc(C(=O)NC(CCc2ccccc2)C(=O)C(N)=O)n1-c1ccncn1]

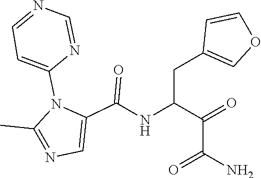 [SMILES: Cc1ncc(C(=O)NC(Cc2ccoc2)C(=O)C(N)=O)n1-c1ccncn1]

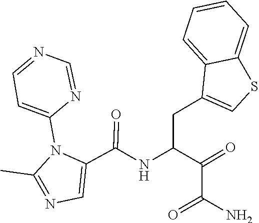 [SMILES: Cc1ncc(C(=O)NC(Cc2csc3ccccc23)C(=O)C(N)=O)n1-c1ccncn1]

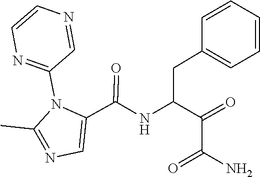 [SMILES: Cc1ncc(C(=O)NC(Cc2ccccc2)C(=O)C(N)=O)n1-c1cnccn1]

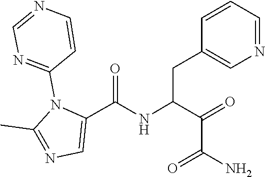 [SMILES: Cc1ncc(C(=O)NC(Cc2cccnc2)C(=O)C(N)=O)n1-c1ccncn1]

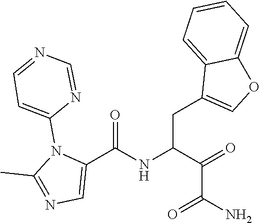 [SMILES: Cc1ncc(C(=O)NC(Cc2coc3ccccc23)C(=O)C(N)=O)n1-c1ccncn1]

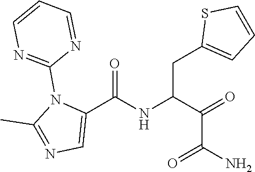 [SMILES: Cc1ncc(C(=O)NC(Cc2cccs2)C(=O)C(N)=O)n1-c1ncccn1]

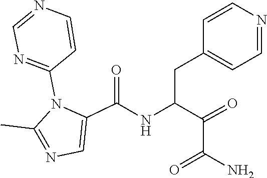 [SMILES: Cc1ncc(C(=O)NC(Cc2ccncc2)C(=O)C(N)=O)n1-c1ccncn1]

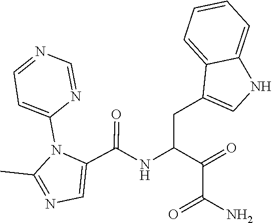 [SMILES: Cc1ncc(C(=O)NC(Cc2c[nH]c3ccccc23)C(=O)C(N)=O)n1-c1ccncn1]

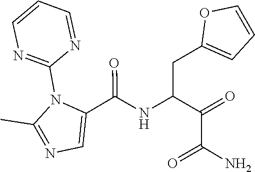 [SMILES: Cc1ncc(C(=O)NC(Cc2ccco2)C(=O)C(N)=O)n1-c1ncccn1]

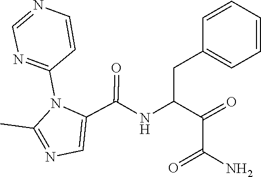 [SMILES: Cc1ncc(C(=O)NC(Cc2ccccc2)C(=O)C(N)=O)n1-c1ccncn1]

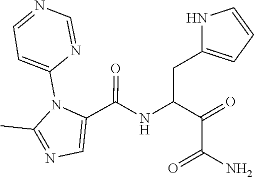 [SMILES: Cc1ncc(C(=O)NC(Cc2ccc[nH]2)C(=O)C(N)=O)n1-c1ccncn1]

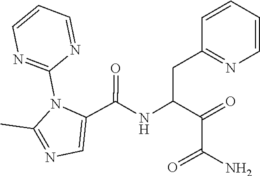 [SMILES: Cc1ncc(C(=O)NC(Cc2ccccn2)C(=O)C(N)=O)n1-c1ncccn1]

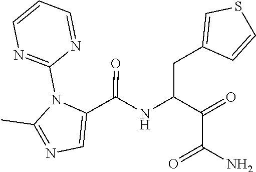 [SMILES: Cc1ncc(C(=O)NC(Cc2ccsc2)C(=O)C(N)=O)n1-c1ncccn1]

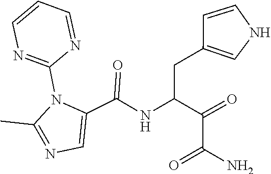 [SMILES: Cc1ncc(C(=O)NC(Cc2cc[nH]c2)C(=O)C(N)=O)n1-c1ncccn1]

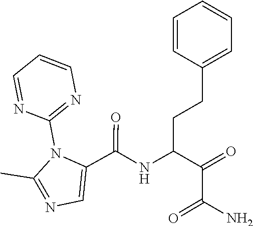 [SMILES: Cc1ncc(C(=O)NC(CCc2ccccc2)C(=O)C(N)=O)n1-c1ncccn1]

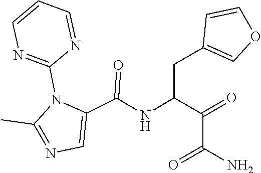 [SMILES: Cc1ncc(C(=O)NC(Cc2ccoc2)C(=O)C(N)=O)n1-c1ncccn1]

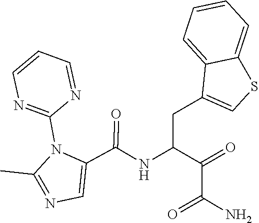 [SMILES: Cc1ncc(C(=O)NC(Cc2csc3ccccc23)C(=O)C(N)=O)n1-c1ncccn1]

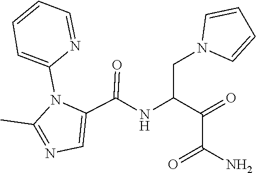 [SMILES: Cc1ncc(C(=O)NC(Cn2cccc2)C(=O)C(N)=O)n1-c1ccccn1]

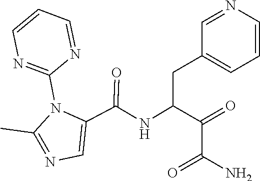 [SMILES: Cc1ncc(C(=O)NC(Cc2cccnc2)C(=O)C(N)=O)n1-c1ncccn1]

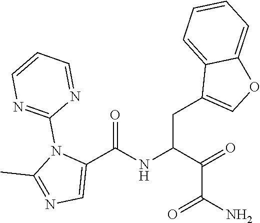 [SMILES: Cc1ncc(C(=O)NC(Cc2coc3ccccc23)C(=O)C(N)=O)n1-c1ncccn1]

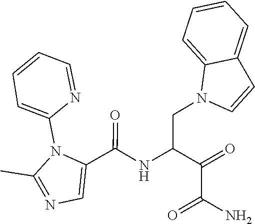 [SMILES: Cc1ncc(C(=O)NC(Cn2ccc3ccccc32)C(=O)C(N)=O)n1-c1ccccn1]

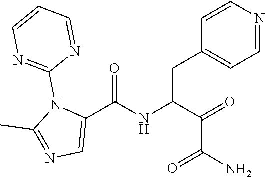 [SMILES: Cc1ncc(C(=O)NC(Cc2ccncc2)C(=O)C(N)=O)n1-c1ncccn1]

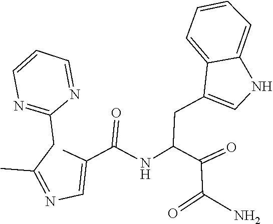 [SMILES: C/C(Cc1ncccn1)=N/C=C(\C)C(=O)NC(Cc1c[nH]c2ccccc12)C(=O)C(N)=O]

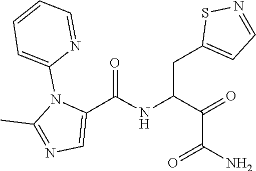 [SMILES: Cc1ncc(C(=O)NC(Cc2ccns2)C(=O)C(N)=O)n1-c1ccccn1]

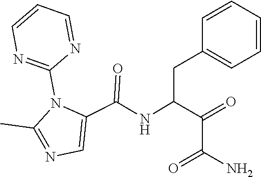 [SMILES: Cc1ncc(C(=O)NC(Cc2ccccc2)C(=O)C(N)=O)n1-c1ncccn1]

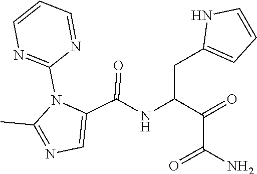 [SMILES: Cc1ncc(C(=O)NC(Cc2ccc[nH]2)C(=O)C(N)=O)n1-c1ncccn1]

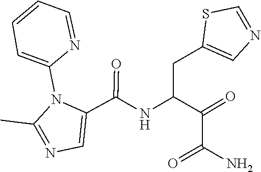 [SMILES: Cc1ncc(C(=O)NC(Cc2cncs2)C(=O)C(N)=O)n1-c1ccccn1]

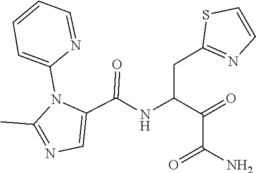 [SMILES: Cc1ncc(C(=O)NC(Cc2nccs2)C(=O)C(N)=O)n1-c1ccccn1]

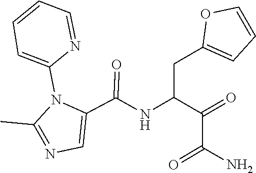 [SMILES: Cc1ncc(C(=O)NC(Cc2ccco2)C(=O)C(N)=O)n1-c1ccccn1]

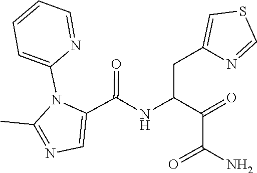 [SMILES: Cc1ncc(C(=O)NC(Cc2cscn2)C(=O)C(N)=O)n1-c1ccccn1]

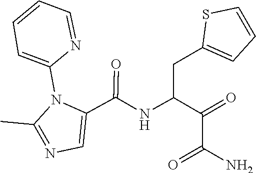 [SMILES: Cc1ncc(C(=O)NC(Cc2cccs2)C(=O)C(N)=O)n1-c1ccccn1]

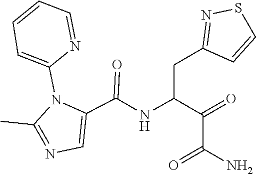 [SMILES: Cc1ncc(C(=O)NC(Cc2ccsn2)C(=O)C(N)=O)n1-c1ccccn1]

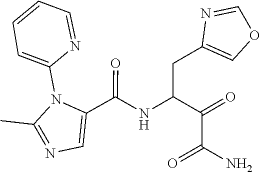 [SMILES: Cc1ncc(C(=O)NC(Cc2cocn2)C(=O)C(N)=O)n1-c1ccccn1]

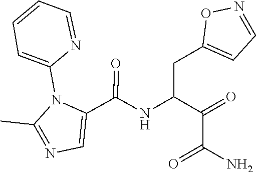 [SMILES: Cc1ncc(C(=O)NC(Cc2ccno2)C(=O)C(N)=O)n1-c1ccccn1]

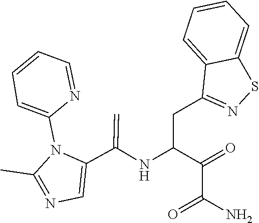 [SMILES: C=C(NC(Cc1nsc2ccccc12)C(=O)C(N)=O)c1cnc(C)n1-c1ccccn1]

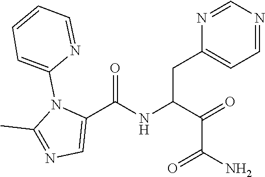 [SMILES: Cc1ncc(C(=O)NC(Cc2ccncn2)C(=O)C(N)=O)n1-c1ccccn1]

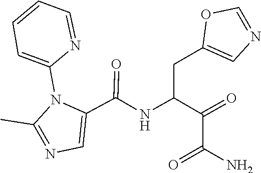 [SMILES: Cc1ncc(C(=O)NC(Cc2cnco2)C(=O)C(N)=O)n1-c1ccccn1]

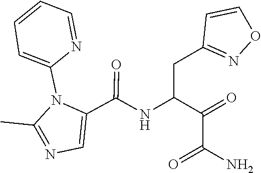 [SMILES: Cc1ncc(C(=O)NC(Cc2ccon2)C(=O)C(N)=O)n1-c1ccccn1]

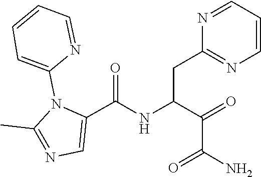 [SMILES: Cc1ncc(C(=O)NC(Cc2ncccn2)C(=O)C(N)=O)n1-c1ccccn1]

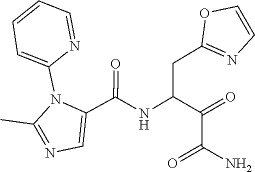 [SMILES: Cc1ncc(C(=O)NC(Cc2ncco2)C(=O)C(N)=O)n1-c1ccccn1]

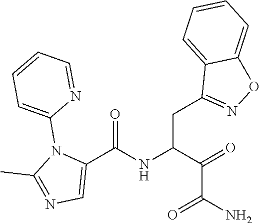 [SMILES: Cc1ncc(C(=O)NC(Cc2noc3ccccc23)C(=O)C(N)=O)n1-c1ccccn1]

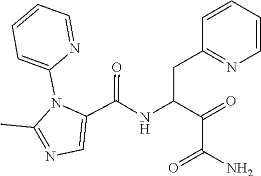 [SMILES: Cc1ncc(C(=O)NC(Cc2ccccn2)C(=O)C(N)=O)n1-c1ccccn1]

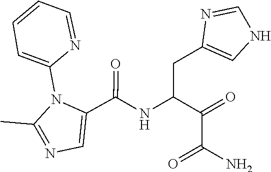 [SMILES: Cc1ncc(C(=O)NC(Cc2c[nH]cn2)C(=O)C(N)=O)n1-c1ccccn1]

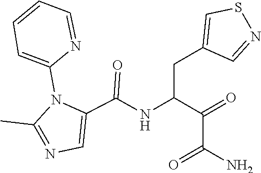 [SMILES: Cc1ncc(C(=O)NC(Cc2cnsc2)C(=O)C(N)=O)n1-c1ccccn1]

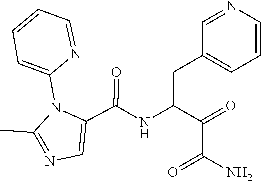 [SMILES: Cc1ncc(C(=O)NC(Cc2cccnc2)C(=O)C(N)=O)n1-c1ccccn1]

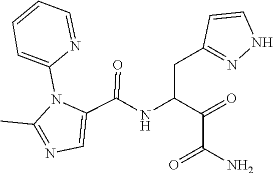 [SMILES: Cc1ncc(C(=O)NC(Cc2cc[nH]n2)C(=O)C(N)=O)n1-c1ccccn1]

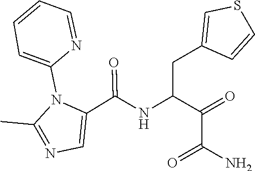 [SMILES: Cc1ncc(C(=O)NC(Cc2ccsc2)C(=O)C(N)=O)n1-c1ccccn1]

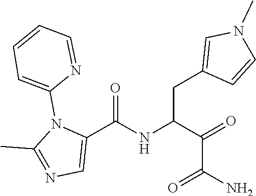 [SMILES: Cc1ncc(C(=O)NC(Cc2ccn(C)c2)C(=O)C(N)=O)n1-c1ccccn1]

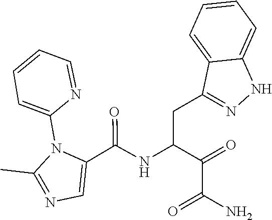 [SMILES: Cc1ncc(C(=O)NC(Cc2n[nH]c3ccccc23)C(=O)C(N)=O)n1-c1ccccn1]

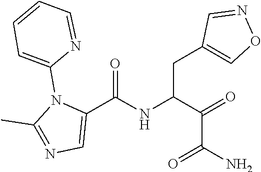 [SMILES: Cc1ncc(C(=O)NC(Cc2cnoc2)C(=O)C(N)=O)n1-c1ccccn1]

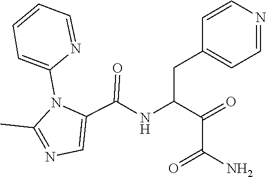 [SMILES: Cc1ncc(C(=O)NC(Cc2ccncc2)C(=O)C(N)=O)n1-c1ccccn1]

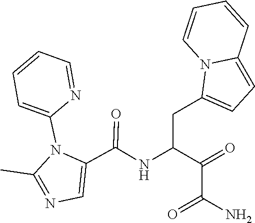 [SMILES: Cc1ncc(C(=O)NC(Cc2ccc3ccccn23)C(=O)C(N)=O)n1-c1ccccn1]

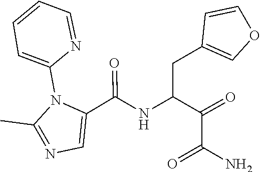 [SMILES: Cc1ncc(C(=O)NC(Cc2ccoc2)C(=O)C(N)=O)n1-c1ccccn1]

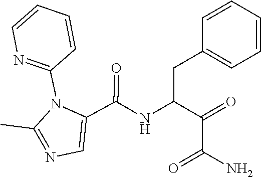 [SMILES: Cc1ncc(C(=O)NC(Cc2ccccc2)C(=O)C(N)=O)n1-c1ccccn1]

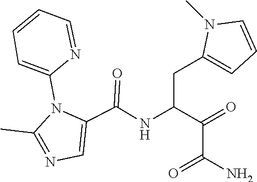 [SMILES: Cc1ncc(C(=O)NC(Cc2cccn2C)C(=O)C(N)=O)n1-c1ccccn1]

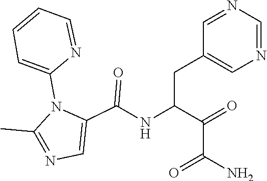 [SMILES: Cc1ncc(C(=O)NC(Cc2cncnc2)C(=O)C(N)=O)n1-c1ccccn1]

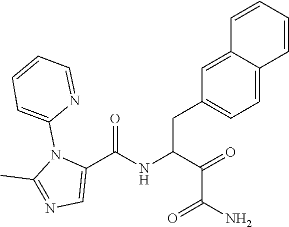 [SMILES: Cc1ncc(C(=O)NC(Cc2ccc3ccccc3c2)C(=O)C(N)=O)n1-c1ccccn1]

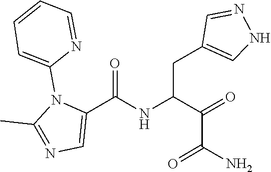 [SMILES: Cc1ncc(C(=O)NC(Cc2cn[nH]c2)C(=O)C(N)=O)n1-c1ccccn1]

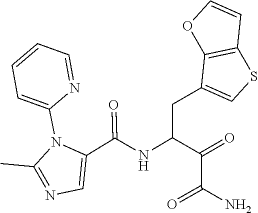 [SMILES: Cc1ncc(C(=O)NC(Cc2csc3ccoc23)C(=O)C(N)=O)n1-c1ccccn1]

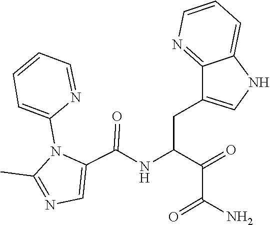 [SMILES: Cc1ncc(C(=O)NC(Cc2c[nH]c3cccnc23)C(=O)C(N)=O)n1-c1ccccn1]

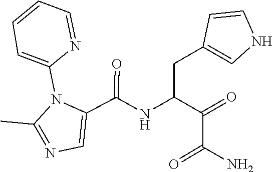 [SMILES: Cc1ncc(C(=O)NC(Cc2cc[nH]c2)C(=O)C(N)=O)n1-c1ccccn1]

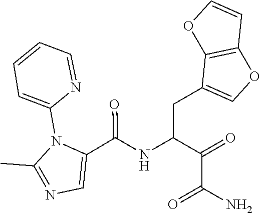 [SMILES: Cc1ncc(C(=O)NC(Cc2coc3ccoc23)C(=O)C(N)=O)n1-c1ccccn1]

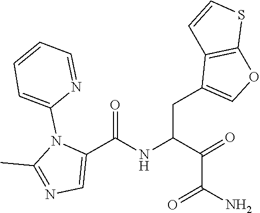 [SMILES: Cc1ncc(C(=O)NC(Cc2coc3sccc23)C(=O)C(N)=O)n1-c1ccccn1]

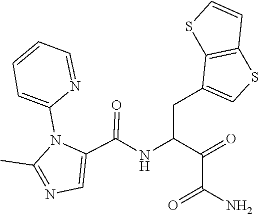 [SMILES: Cc1ncc(C(=O)NC(Cc2csc3ccsc23)C(=O)C(N)=O)n1-c1ccccn1]

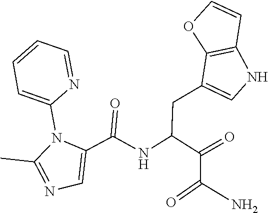 [SMILES: Cc1ncc(C(=O)NC(Cc2c[nH]c3ccoc23)C(=O)C(N)=O)n1-c1ccccn1]

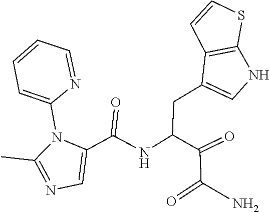 [SMILES: Cc1ncc(C(=O)NC(Cc2c[nH]c3sccc23)C(=O)C(N)=O)n1-c1ccccn1]

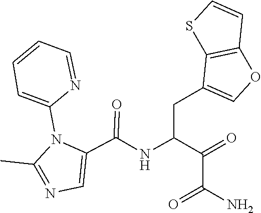 [SMILES: Cc1ncc(C(=O)NC(Cc2coc3ccsc23)C(=O)C(N)=O)n1-c1ccccn1]

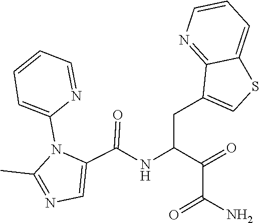 [SMILES: Cc1ncc(C(=O)NC(Cc2csc3cccnc23)C(=O)C(N)=O)n1-c1ccccn1]

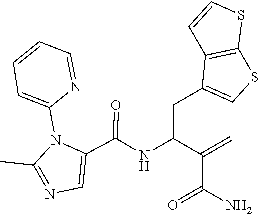 [SMILES: C=C(C(N)=O)C(Cc1csc2sccc12)NC(=O)c1cnc(C)n1-c1ccccn1]

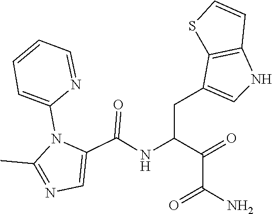 [SMILES: Cc1ncc(C(=O)NC(Cc2c[nH]c3ccsc23)C(=O)C(N)=O)n1-c1ccccn1]

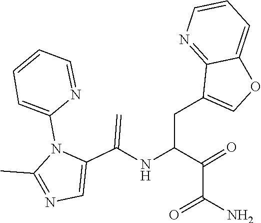 [SMILES: C=C(NC(Cc1coc2cccnc12)C(=O)C(N)=O)c1cnc(C)n1-c1ccccn1]

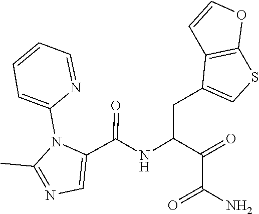 [SMILES: Cc1ncc(C(=O)NC(Cc2csc3occc23)C(=O)C(N)=O)n1-c1ccccn1]

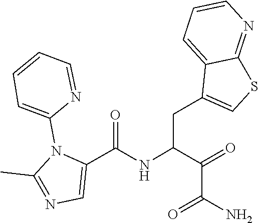 [SMILES: Cc1ncc(C(=O)NC(Cc2csc3ncccc23)C(=O)C(N)=O)n1-c1ccccn1]

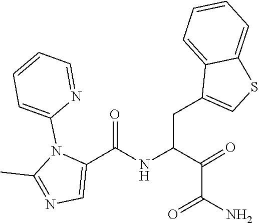 [SMILES: Cc1ncc(C(=O)NC(Cc2csc3ccccc23)C(=O)C(N)=O)n1-c1ccccn1]

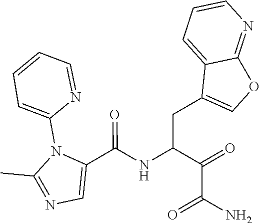 [SMILES: Cc1ncc(C(=O)NC(Cc2coc3ncccc23)C(=O)C(N)=O)n1-c1ccccn1]

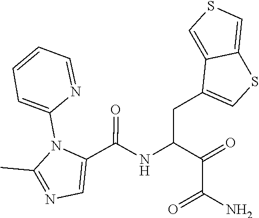 [SMILES: Cc1ncc(C(=O)NC(Cc2csc3cscc23)C(=O)C(N)=O)n1-c1ccccn1]

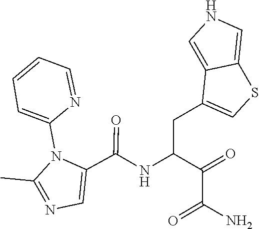 [SMILES: Cc1ncc(C(=O)NC(Cc2csc3c[nH]cc23)C(=O)C(N)=O)n1-c1ccccn1]

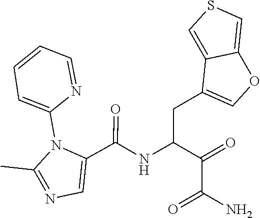 [SMILES: Cc1ncc(C(=O)NC(Cc2coc3cscc23)C(=O)C(N)=O)n1-c1ccccn1]

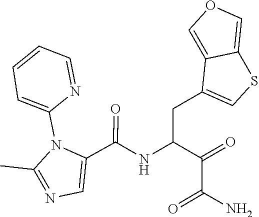 [SMILES: Cc1ncc(C(=O)NC(Cc2csc3cocc23)C(=O)C(N)=O)n1-c1ccccn1]

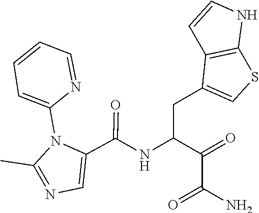 [SMILES: Cc1ncc(C(=O)NC(Cc2csc3[nH]ccc23)C(=O)C(N)=O)n1-c1ccccn1]

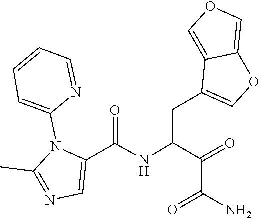 [SMILES: Cc1ncc(C(=O)NC(Cc2coc3cocc23)C(=O)C(N)=O)n1-c1ccccn1]

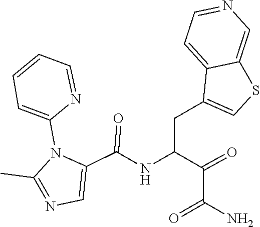 [SMILES: Cc1ncc(C(=O)NC(Cc2csc3cnccc23)C(=O)C(N)=O)n1-c1ccccn1]

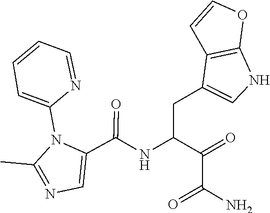 [SMILES: Cc1ncc(C(=O)NC(Cc2c[nH]c3occc23)C(=O)C(N)=O)n1-c1ccccn1]

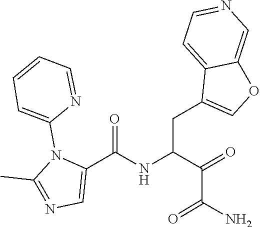 [SMILES: Cc1ncc(C(=O)NC(Cc2coc3cnccc23)C(=O)C(N)=O)n1-c1ccccn1]

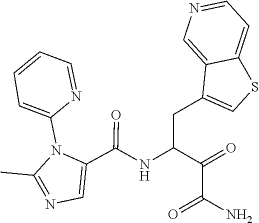 [SMILES: Cc1ncc(C(=O)NC(Cc2csc3ccncc23)C(=O)C(N)=O)n1-c1ccccn1]

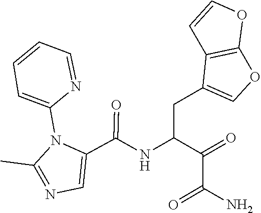 [SMILES: Cc1ncc(C(=O)NC(Cc2coc3occc23)C(=O)C(N)=O)n1-c1ccccn1]

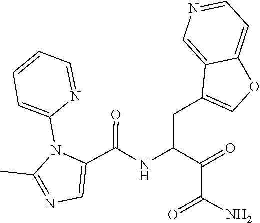 [SMILES: Cc1ncc(C(=O)NC(Cc2coc3ccncc23)C(=O)C(N)=O)n1-c1ccccn1]

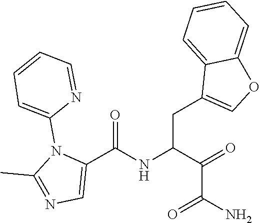 [SMILES: Cc1ncc(C(=O)NC(Cc2coc3ccccc23)C(=O)C(N)=O)n1-c1ccccn1]

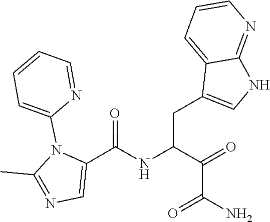 [SMILES: Cc1ncc(C(=O)NC(Cc2c[nH]c3ncccc23)C(=O)C(N)=O)n1-c1ccccn1]

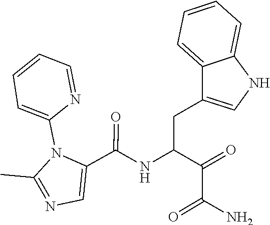 [SMILES: Cc1ncc(C(=O)NC(Cc2c[nH]c3ccccc23)C(=O)C(N)=O)n1-c1ccccn1]

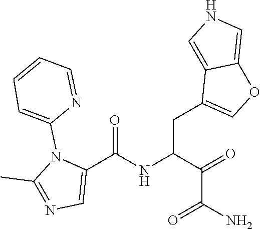 [SMILES: Cc1ncc(C(=O)NC(Cc2coc3c[nH]cc23)C(=O)C(N)=O)n1-c1ccccn1]

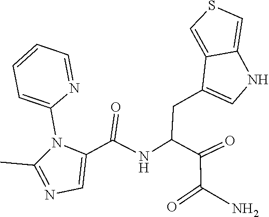 [SMILES: Cc1ncc(C(=O)NC(Cc2c[nH]c3cscc23)C(=O)C(N)=O)n1-c1ccccn1]

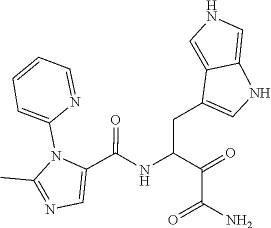 [SMILES: Cc1ncc(C(=O)NC(Cc2c[nH]c3c[nH]cc23)C(=O)C(N)=O)n1-c1ccccn1]

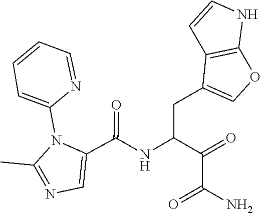 [SMILES: Cc1ncc(C(=O)NC(Cc2coc3[nH]ccc23)C(=O)C(N)=O)n1-c1ccccn1]

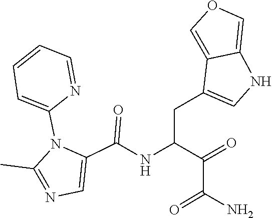 [SMILES: Cc1ncc(C(=O)NC(Cc2c[nH]c3cocc23)C(=O)C(N)=O)n1-c1ccccn1]

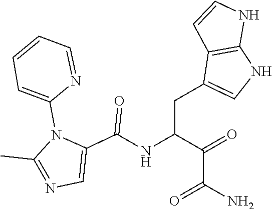 [SMILES: Cc1ncc(C(=O)NC(Cc2c[nH]c3[nH]ccc23)C(=O)C(N)=O)n1-c1ccccn1]

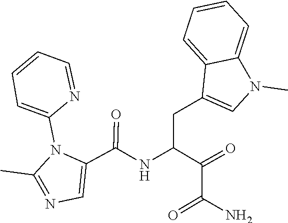

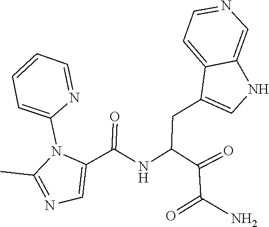 [SMILES: Cc1ncc(C(=O)NC(Cc2c[nH]c3cnccc23)C(=O)C(N)=O)n1-c1ccccn1]

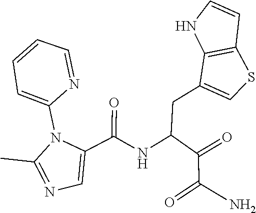 [SMILES: Cc1ncc(C(=O)NC(Cc2csc3cc[nH]c23)C(=O)C(N)=O)n1-c1ccccn1]

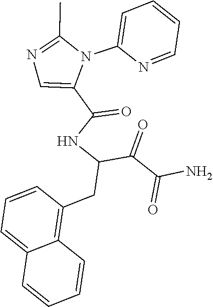 [SMILES: Cc1ncc(C(=O)NC(Cc2cccc3ccccc23)C(=O)C(N)=O)n1-c1ccccn1]

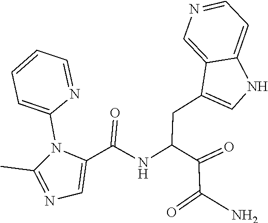 [SMILES: Cc1ncc(C(=O)NC(Cc2c[nH]c3ccncc23)C(=O)C(N)=O)n1-c1ccccn1]

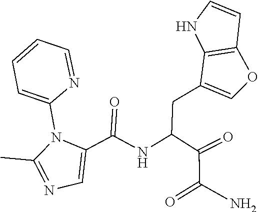 [SMILES: Cc1ncc(C(=O)NC(Cc2coc3cc[nH]c23)C(=O)C(N)=O)n1-c1ccccn1]

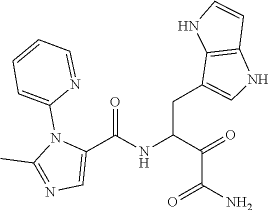 [SMILES: Cc1ncc(C(=O)NC(Cc2c[nH]c3cc[nH]c23)C(=O)C(N)=O)n1-c1ccccn1]

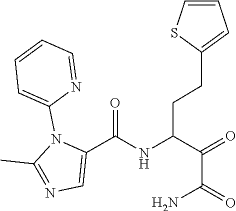 [SMILES: Cc1ncc(C(=O)NC(CCc2cccs2)C(=O)C(N)=O)n1-c1ccccn1]

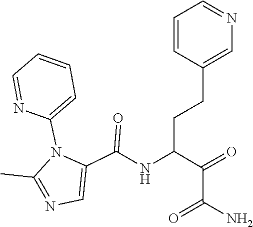 [SMILES: Cc1ncc(C(=O)NC(CCc2cccnc2)C(=O)C(N)=O)n1-c1ccccn1]

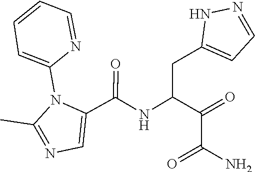 [SMILES: Cc1ncc(C(=O)NC(Cc2ccn[nH]2)C(=O)C(N)=O)n1-c1ccccn1]

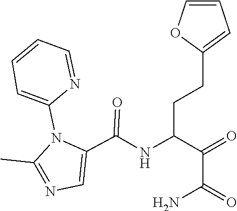 [SMILES: Cc1ncc(C(=O)NC(CCc2ccco2)C(=O)C(N)=O)n1-c1ccccn1]

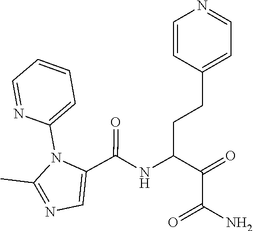 [SMILES: Cc1ncc(C(=O)NC(CCc2ccncc2)C(=O)C(N)=O)n1-c1ccccn1]

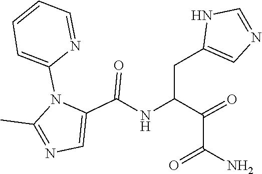 [SMILES: Cc1ncc(C(=O)NC(Cc2cnc[nH]2)C(=O)C(N)=O)n1-c1ccccn1]

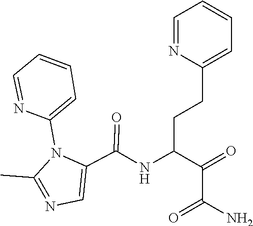 [SMILES: Cc1ncc(C(=O)NC(CCc2ccccn2)C(=O)C(N)=O)n1-c1ccccn1]

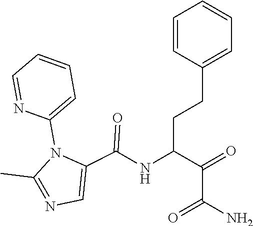 [SMILES: Cc1ncc(C(=O)NC(CCc2ccccc2)C(=O)C(N)=O)n1-c1ccccn1]

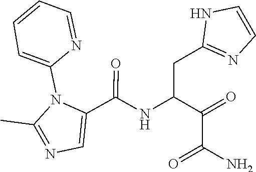 [SMILES: Cc1ncc(C(=O)NC(Cc2ncc[nH]2)C(=O)C(N)=O)n1-c1ccccn1]

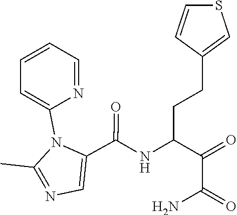 [SMILES: Cc1ncc(C(=O)NC(CCc2ccsc2)C(=O)C(N)=O)n1-c1ccccn1]

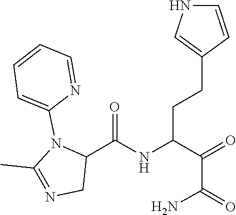 [SMILES: CC1=NCC(C(=O)NC(CCc2cc[nH]c2)C(=O)C(N)=O)N1c1ccccn1]

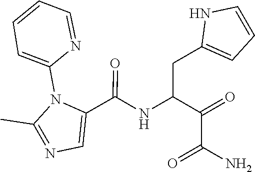 [SMILES: Cc1ncc(C(=O)NC(Cc2ccc[nH]2)C(=O)C(N)=O)n1-c1ccccn1]

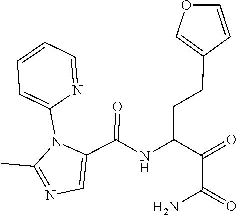 [SMILES: Cc1ncc(C(=O)NC(CCc2ccoc2)C(=O)C(N)=O)n1-c1ccccn1]

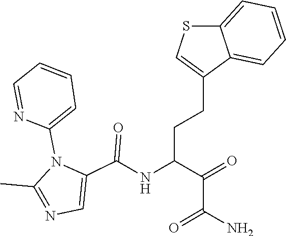 [SMILES: Cc1ncc(C(=O)NC(CCc2csc3ccccc23)C(=O)C(N)=O)n1-c1ccccn1]

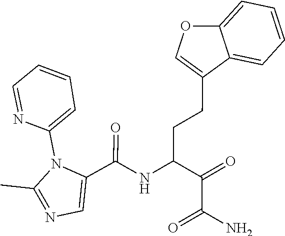 [SMILES: Cc1ncc(C(=O)NC(CCc2coc3ccccc23)C(=O)C(N)=O)n1-c1ccccn1]

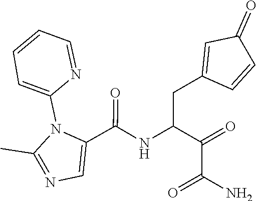 [SMILES: Cc1ncc(C(=O)NC(CC2=CC(=O)C=C2)C(=O)C(N)=O)n1-c1ccccn1]

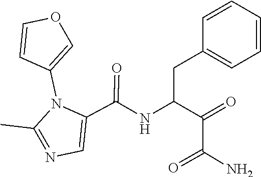 [SMILES: Cc1ncc(C(=O)NC(Cc2ccccc2)C(=O)C(N)=O)n1-c1ccoc1]

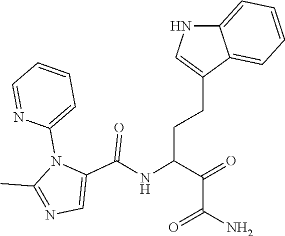 [SMILES: Cc1ncc(C(=O)NC(CCc2c[nH]c3ccccc23)C(=O)C(N)=O)n1-c1ccccn1]

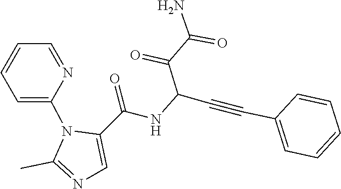 [SMILES: Cc1ncc(C(=O)NC(C#Cc2ccccc2)C(=O)C(N)=O)n1-c1ccccn1]

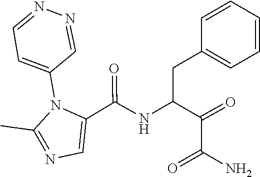 [SMILES: Cc1ncc(C(=O)NC(Cc2ccccc2)C(=O)C(N)=O)n1-c1ccnnc1]

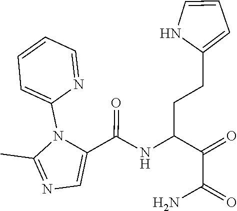 [SMILES: Cc1ncc(C(=O)NC(CCc2ccc[nH]2)C(=O)C(N)=O)n1-c1ccccn1]

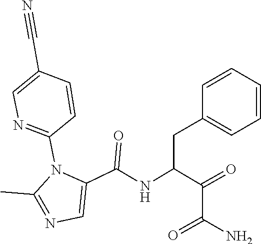 [SMILES: Cc1ncc(C(=O)NC(Cc2ccccc2)C(=O)C(N)=O)n1-c1ccc(C#N)cn1]

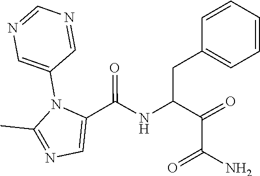 [SMILES: Cc1ncc(C(=O)NC(Cc2ccccc2)C(=O)C(N)=O)n1-c1cncnc1]

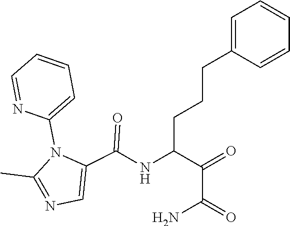 [SMILES: Cc1ncc(C(=O)NC(CCCc2ccccc2)C(=O)C(N)=O)n1-c1ccccn1]

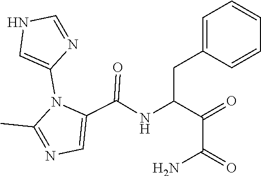 [SMILES: Cc1ncc(C(=O)NC(Cc2ccccc2)C(=O)C(N)=O)n1-c1c[nH]cn1]

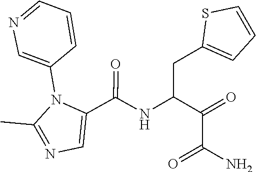 [SMILES: Cc1ncc(C(=O)NC(Cc2cccs2)C(=O)C(N)=O)n1-c1cccnc1]

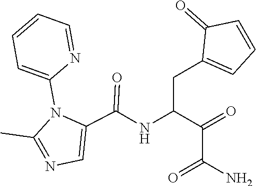 [SMILES: Cc1ncc(C(=O)NC(CC2=CC=CC2=O)C(=O)C(N)=O)n1-c1ccccn1]

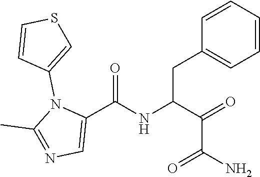 [SMILES: Cc1ncc(C(=O)NC(Cc2ccccc2)C(=O)C(N)=O)n1-c1ccsc1]

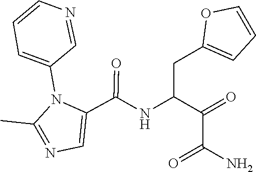 [SMILES: Cc1ncc(C(=O)NC(Cc2ccco2)C(=O)C(N)=O)n1-c1cccnc1]

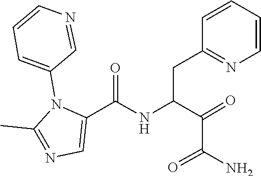 [SMILES: Cc1ncc(C(=O)NC(Cc2ccccn2)C(=O)C(N)=O)n1-c1cccnc1]

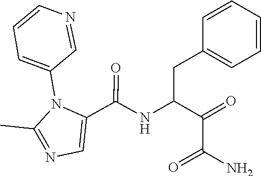 [SMILES: Cc1ncc(C(=O)NC(Cc2ccccc2)C(=O)C(N)=O)n1-c1cccnc1]

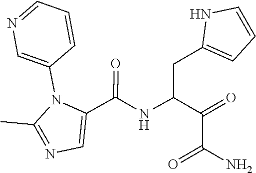 [SMILES: Cc1ncc(C(=O)NC(Cc2ccc[nH]2)C(=O)C(N)=O)n1-c1cccnc1]

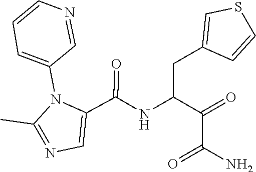 [SMILES: Cc1ncc(C(=O)NC(Cc2ccsc2)C(=O)C(N)=O)n1-c1cccnc1]

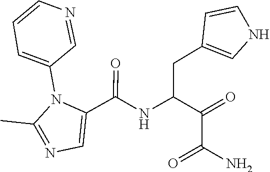 [SMILES: Cc1ncc(C(=O)NC(Cc2cc[nH]c2)C(=O)C(N)=O)n1-c1cccnc1]

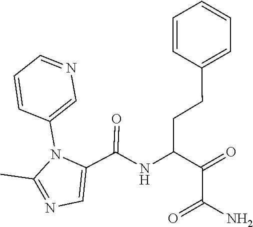 [SMILES: Cc1ncc(C(=O)NC(CCc2ccccc2)C(=O)C(N)=O)n1-c1cccnc1]

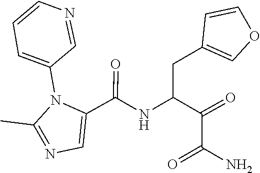 [SMILES: Cc1ncc(C(=O)NC(Cc2ccoc2)C(=O)C(N)=O)n1-c1cccnc1]

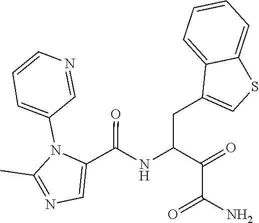 [SMILES: Cc1ncc(C(=O)NC(Cc2csc3ccccc23)C(=O)C(N)=O)n1-c1cccnc1]

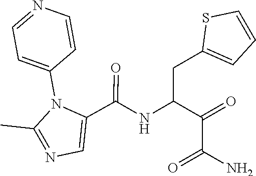 [SMILES: Cc1ncc(C(=O)NC(Cc2cccs2)C(=O)C(N)=O)n1-c1ccncc1]

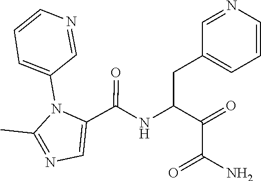 [SMILES: Cc1ncc(C(=O)NC(Cc2cccnc2)C(=O)C(N)=O)n1-c1cccnc1]

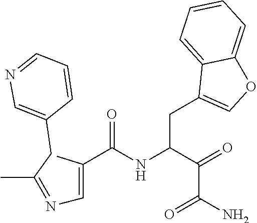 [SMILES: C/C(Cc1cccnc1)=N/C=C(\C)C(=O)NC(Cc1coc2ccccc12)C(=O)C(N)=O]

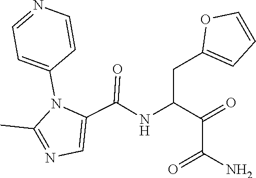 [SMILES: Cc1ncc(C(=O)NC(Cc2ccco2)C(=O)C(N)=O)n1-c1ccncc1]

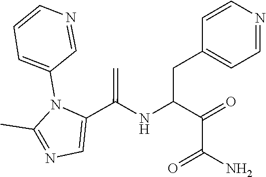 [SMILES: C=C(NC(Cc1ccncc1)C(=O)C(N)=O)c1cnc(C)n1-c1cccnc1]

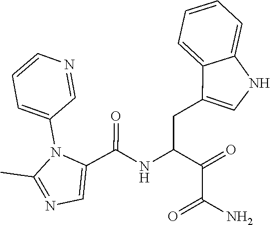 [SMILES: Cc1ncc(C(=O)NC(Cc2c[nH]c3ccccc23)C(=O)C(N)=O)n1-c1cccnc1]

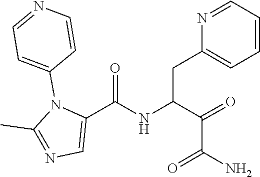 [SMILES: Cc1ncc(C(=O)NC(Cc2ccccn2)C(=O)C(N)=O)n1-c1ccncc1]

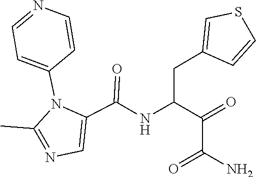 [SMILES: Cc1ncc(C(=O)NC(Cc2ccsc2)C(=O)C(N)=O)n1-c1ccncc1]

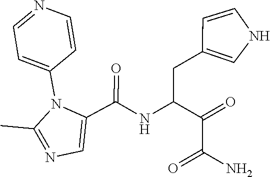 [SMILES: Cc1ncc(C(=O)NC(Cc2cc[nH]c2)C(=O)C(N)=O)n1-c1ccncc1]

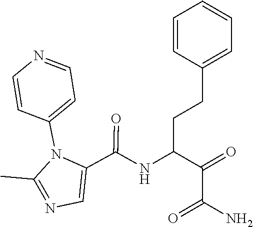 [SMILES: Cc1ncc(C(=O)NC(CCc2ccccc2)C(=O)C(N)=O)n1-c1ccncc1]

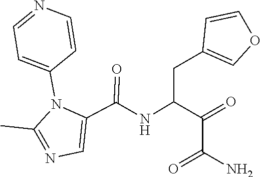 [SMILES: Cc1ncc(C(=O)NC(Cc2ccoc2)C(=O)C(N)=O)n1-c1ccncc1]

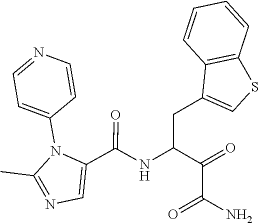 [SMILES: Cc1ncc(C(=O)NC(Cc2csc3ccccc23)C(=O)C(N)=O)n1-c1ccncc1]

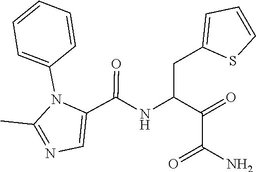 [SMILES: Cc1ncc(C(=O)NC(Cc2cccs2)C(=O)C(N)=O)n1-c1ccccc1]

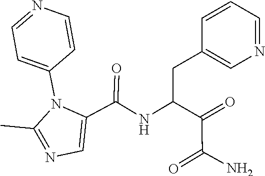 [SMILES: Cc1ncc(C(=O)NC(Cc2cccnc2)C(=O)C(N)=O)n1-c1ccncc1]

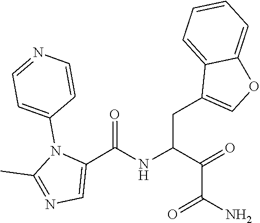 [SMILES: Cc1ncc(C(=O)NC(Cc2coc3ccccc23)C(=O)C(N)=O)n1-c1ccncc1]

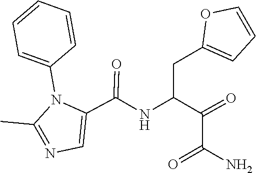 [SMILES: Cc1ncc(C(=O)NC(Cc2ccco2)C(=O)C(N)=O)n1-c1ccccc1]

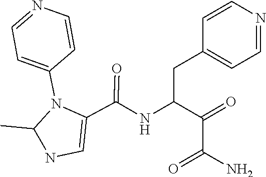

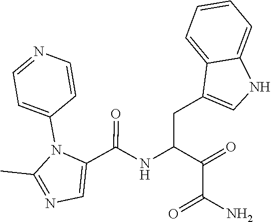 [SMILES: Cc1ncc(C(=O)NC(Cc2c[nH]c3ccccc23)C(=O)C(N)=O)n1-c1ccncc1]

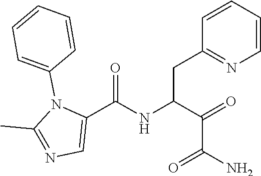 [SMILES: Cc1ncc(C(=O)NC(Cc2ccccn2)C(=O)C(N)=O)n1-c1ccccc1]

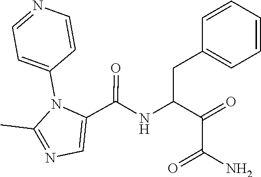 [SMILES: Cc1ncc(C(=O)NC(Cc2ccccc2)C(=O)C(N)=O)n1-c1ccncc1]

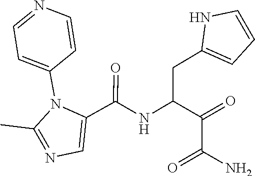 [SMILES: Cc1ncc(C(=O)NC(Cc2ccc[nH]2)C(=O)C(N)=O)n1-c1ccncc1]

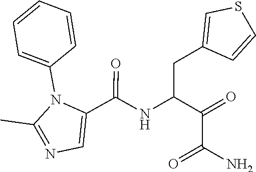 [SMILES: Cc1ncc(C(=O)NC(Cc2ccsc2)C(=O)C(N)=O)n1-c1ccccc1]

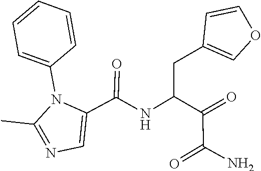 [SMILES: Cc1ncc(C(=O)NC(Cc2ccoc2)C(=O)C(N)=O)n1-c1ccccc1]

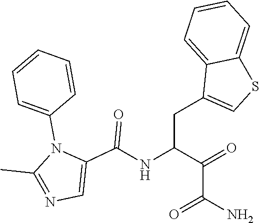 [SMILES: Cc1ncc(C(=O)NC(Cc2csc3ccccc23)C(=O)C(N)=O)n1-c1ccccc1]

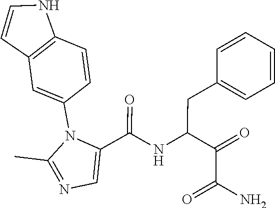 [SMILES: Cc1ncc(C(=O)NC(Cc2ccccc2)C(=O)C(N)=O)n1-c1ccc2[nH]ccc2c1]

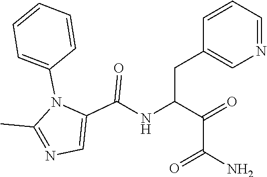 [SMILES: Cc1ncc(C(=O)NC(Cc2cccnc2)C(=O)C(N)=O)n1-c1ccccc1]

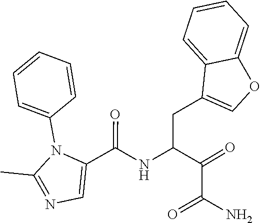 [SMILES: Cc1ncc(C(=O)NC(Cc2coc3ccccc23)C(=O)C(N)=O)n1-c1ccccc1]

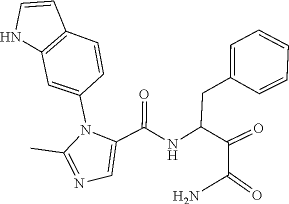 [SMILES: Cc1ncc(C(=O)NC(Cc2ccccc2)C(=O)C(N)=O)n1-c1ccc2cc[nH]c2c1]

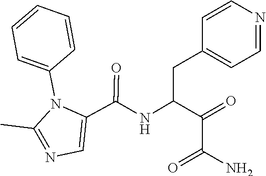 [SMILES: Cc1ncc(C(=O)NC(Cc2ccncc2)C(=O)C(N)=O)n1-c1ccccc1]

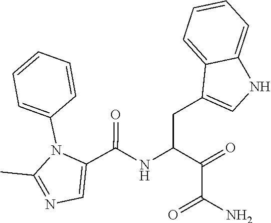 [SMILES: Cc1ncc(C(=O)NC(Cc2c[nH]c3ccccc23)C(=O)C(N)=O)n1-c1ccccc1]

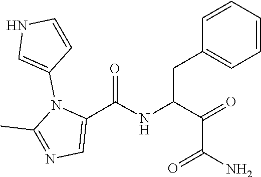 [SMILES: Cc1ncc(C(=O)NC(Cc2ccccc2)C(=O)C(N)=O)n1-c1cc[nH]c1]

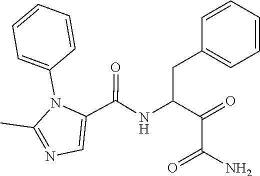 [SMILES: Cc1ncc(C(=O)NC(Cc2ccccc2)C(=O)C(N)=O)n1-c1ccccc1]

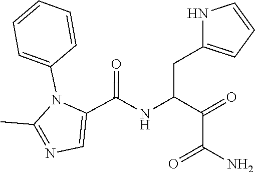 [SMILES: Cc1ncc(C(=O)NC(Cc2ccc[nH]2)C(=O)C(N)=O)n1-c1ccccc1]

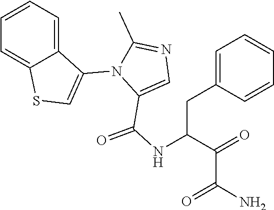 [SMILES: Cc1ncc(C(=O)NC(Cc2ccccc2)C(=O)C(N)=O)n1-c1csc2ccccc12]

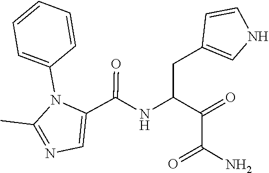 [SMILES: Cc1ncc(C(=O)NC(Cc2cc[nH]c2)C(=O)C(N)=O)n1-c1ccccc1]

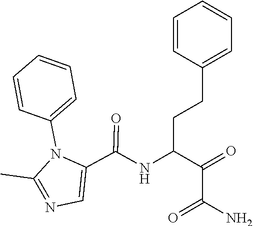 [SMILES: Cc1ncc(C(=O)NC(CCc2ccccc2)C(=O)C(N)=O)n1-c1ccccc1]

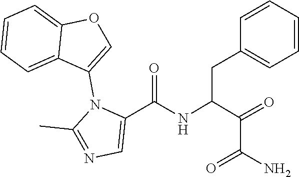 [SMILES: Cc1ncc(C(=O)NC(Cc2ccccc2)C(=O)C(N)=O)n1-c1coc2ccccc12]

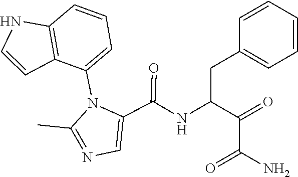 [SMILES: Cc1ncc(C(=O)NC(Cc2ccccc2)C(=O)C(N)=O)n1-c1cccc2[nH]ccc12]

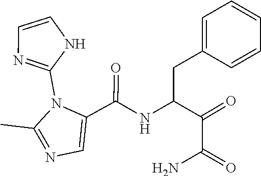 [SMILES: Cc1ncc(C(=O)NC(Cc2ccccc2)C(=O)C(N)=O)n1-c1ncc[nH]1]

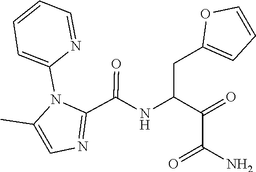 [SMILES: Cc1cnc(C(=O)NC(Cc2ccco2)C(=O)C(N)=O)n1-c1ccccn1]

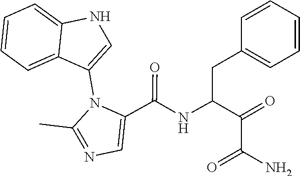 [SMILES: Cc1ncc(C(=O)NC(Cc2ccccc2)C(=O)C(N)=O)n1-c1c[nH]c2ccccc12]

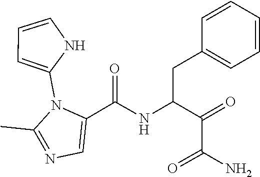 [SMILES: Cc1ncc(C(=O)NC(Cc2ccccc2)C(=O)C(N)=O)n1-c1ccc[nH]1]

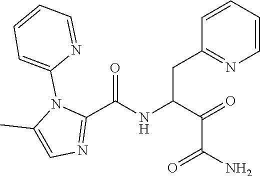 [SMILES: Cc1cnc(C(=O)NC(Cc2ccccn2)C(=O)C(N)=O)n1-c1ccccn1]

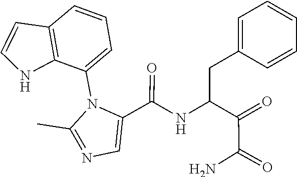 [SMILES: Cc1ncc(C(=O)NC(Cc2ccccc2)C(=O)C(N)=O)n1-c1cccc2cc[nH]c12]

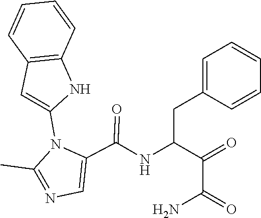 [SMILES: Cc1ncc(C(=O)NC(Cc2ccccc2)C(=O)C(N)=O)n1-c1cc2ccccc2[nH]1]

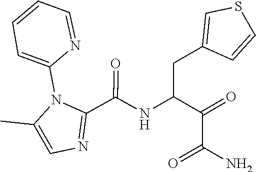 [SMILES: Cc1cnc(C(=O)NC(Cc2ccsc2)C(=O)C(N)=O)n1-c1ccccn1]

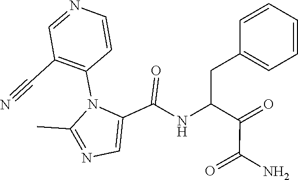 [SMILES: Cc1ncc(C(=O)NC(Cc2ccccc2)C(=O)C(N)=O)n1-c1ccncc1C#N]

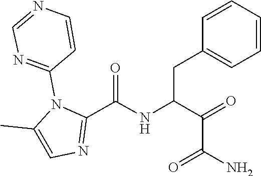 [SMILES: Cc1cnc(C(=O)NC(Cc2ccccc2)C(=O)C(N)=O)n1-c1ccncn1]

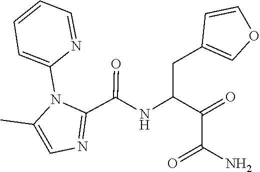 [SMILES: Cc1cnc(C(=O)NC(Cc2ccoc2)C(=O)C(N)=O)n1-c1ccccn1]

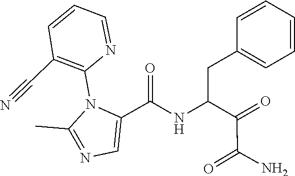 [SMILES: Cc1ncc(C(=O)NC(Cc2ccccc2)C(=O)C(N)=O)n1-c1ncccc1C#N]

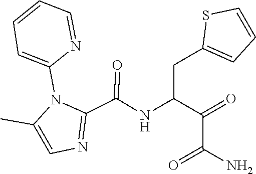 [SMILES: Cc1cnc(C(=O)NC(Cc2cccs2)C(=O)C(N)=O)n1-c1ccccn1]

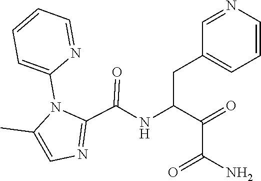 [SMILES: Cc1cnc(C(=O)NC(Cc2cccnc2)C(=O)C(N)=O)n1-c1ccccn1]

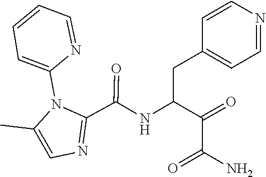 [SMILES: Cc1cnc(C(=O)NC(Cc2ccncc2)C(=O)C(N)=O)n1-c1ccccn1]

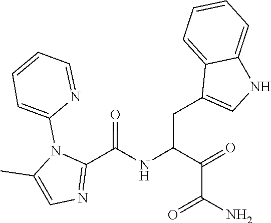 [SMILES: Cc1cnc(C(=O)NC(Cc2c[nH]c3ccccc23)C(=O)C(N)=O)n1-c1ccccn1]

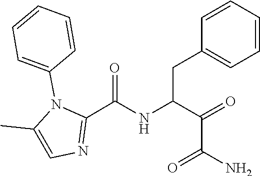 [SMILES: Cc1cnc(C(=O)NC(Cc2ccccc2)C(=O)C(N)=O)n1-c1ccccc1]

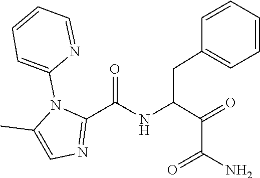 [SMILES: Cc1cnc(C(=O)NC(Cc2ccccc2)C(=O)C(N)=O)n1-c1ccccn1]

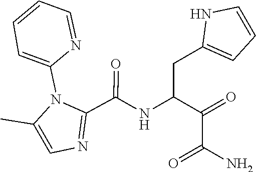 [SMILES: Cc1cnc(C(=O)NC(Cc2ccc[nH]2)C(=O)C(N)=O)n1-c1ccccn1]

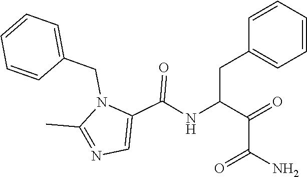 [SMILES: Cc1ncc(C(=O)NC(Cc2ccccc2)C(=O)C(N)=O)n1Cc1ccccc1]

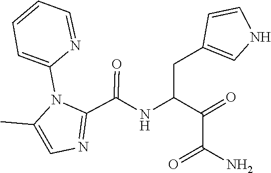 [SMILES: Cc1cnc(C(=O)NC(Cc2cc[nH]c2)C(=O)C(N)=O)n1-c1ccccn1]

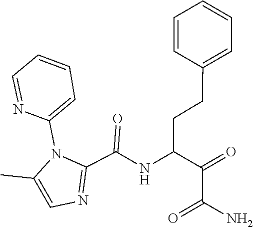 [SMILES: Cc1cnc(C(=O)NC(CCc2ccccc2)C(=O)C(N)=O)n1-c1ccccn1]

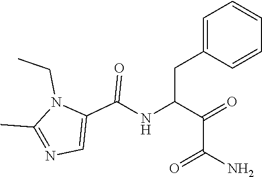 [SMILES: CCn1c(C(=O)NC(Cc2ccccc2)C(=O)C(N)=O)cnc1C]

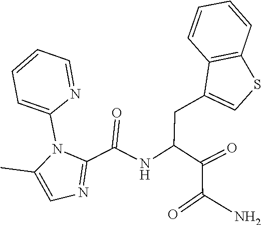 [SMILES: Cc1cnc(C(=O)NC(Cc2csc3ccccc23)C(=O)C(N)=O)n1-c1ccccn1]

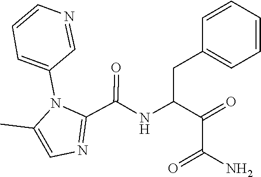 [SMILES: Cc1cnc(C(=O)NC(Cc2ccccc2)C(=O)C(N)=O)n1-c1cccnc1]

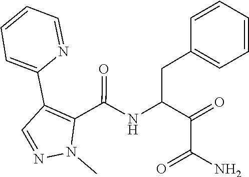 [SMILES: Cn1ncc(-c2ccccn2)c1C(=O)NC(Cc1ccccc1)C(=O)C(N)=O]

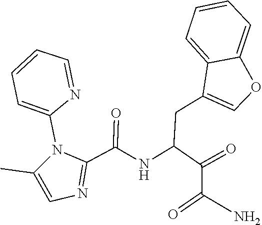 [SMILES: Cc1cnc(C(=O)NC(Cc2coc3ccccc23)C(=O)C(N)=O)n1-c1ccccn1]

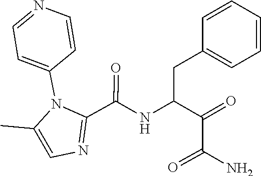 [SMILES: Cc1cnc(C(=O)NC(Cc2ccccc2)C(=O)C(N)=O)n1-c1ccncc1]

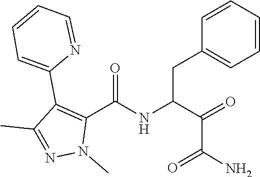 [SMILES: Cc1nn(C)c(C(=O)NC(Cc2ccccc2)C(=O)C(N)=O)c1-c1ccccn1]

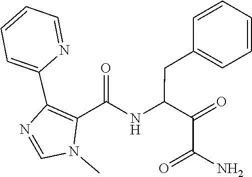 [SMILES: Cn1cnc(-c2ccccn2)c1C(=O)NC(Cc1ccccc1)C(=O)C(N)=O]

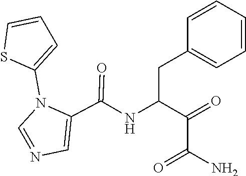 [SMILES: NC(=O)C(=O)C(Cc1ccccc1)NC(=O)c1cncn1-c1cccs1]

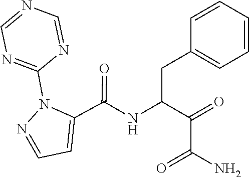 [SMILES: NC(=O)C(=O)C(Cc1ccccc1)NC(=O)c1ccnn1-c1ncncn1]

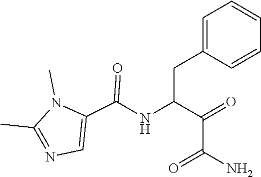 [SMILES: Cc1ncc(C(=O)NC(Cc2ccccc2)C(=O)C(N)=O)n1C]

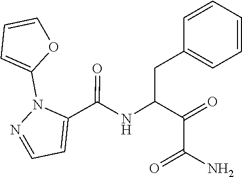 [SMILES: NC(=O)C(=O)C(Cc1ccccc1)NC(=O)c1ccnn1-c1ccco1]

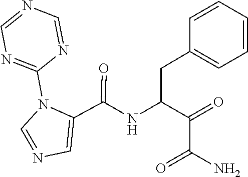 [SMILES: NC(=O)C(=O)C(Cc1ccccc1)NC(=O)c1cncn1-c1ncncn1]

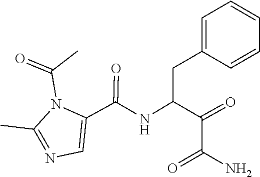 [SMILES: CC(=O)n1c(C(=O)NC(Cc2ccccc2)C(=O)C(N)=O)cnc1C]

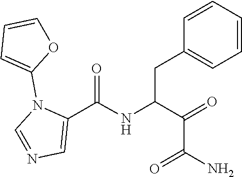 [SMILES: NC(=O)C(=O)C(Cc1ccccc1)NC(=O)c1cncn1-c1ccco1]

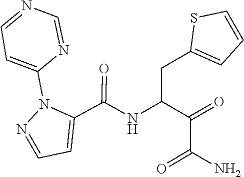 [SMILES: NC(=O)C(=O)C(Cc1cccs1)NC(=O)c1ccnn1-c1ccncn1]

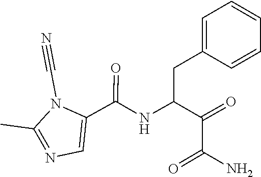 [SMILES: Cc1ncc(C(=O)NC(Cc2ccccc2)C(=O)C(N)=O)n1C#N]

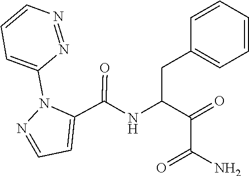 [SMILES: NC(=O)C(=O)C(Cc1ccccc1)NC(=O)c1ccnn1-c1cccnn1]

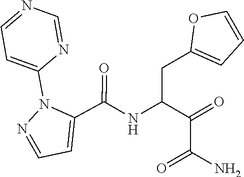 [SMILES: NC(=O)C(=O)C(Cc1ccco1)NC(=O)c1ccnn1-c1ccncn1]

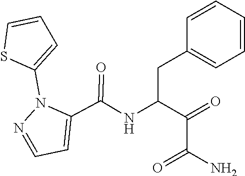 [SMILES: NC(=O)C(=O)C(Cc1ccccc1)NC(=O)c1ccnn1-c1cccs1]

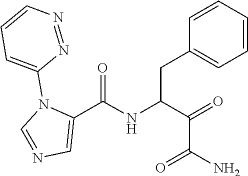 [SMILES: NC(=O)C(=O)C(Cc1ccccc1)NC(=O)c1cncn1-c1cccnn1]

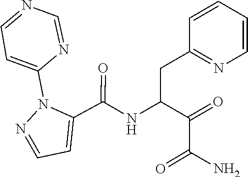 [SMILES: NC(=O)C(=O)C(Cc1ccccn1)NC(=O)c1ccnn1-c1ccncn1]

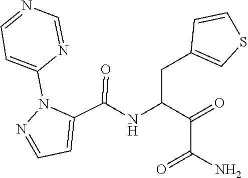 [SMILES: NC(=O)C(=O)C(Cc1ccsc1)NC(=O)c1ccnn1-c1ccncn1]

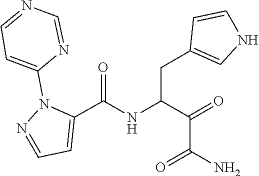 [SMILES: NC(=O)C(=O)C(Cc1cc[nH]c1)NC(=O)c1ccnn1-c1ccncn1]

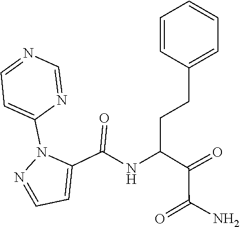 [SMILES: NC(=O)C(=O)C(CCc1ccccc1)NC(=O)c1ccnn1-c1ccncn1]

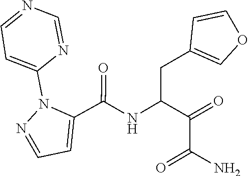 [SMILES: NC(=O)C(=O)C(Cc1ccoc1)NC(=O)c1ccnn1-c1ccncn1]

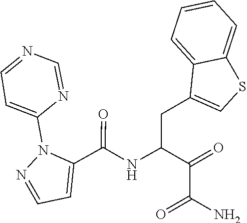 [SMILES: NC(=O)C(=O)C(Cc1csc2ccccc12)NC(=O)c1ccnn1-c1ccncn1]

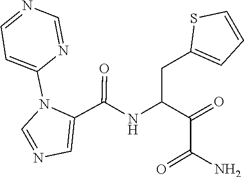 [SMILES: NC(=O)C(=O)C(Cc1cccs1)NC(=O)c1cncn1-c1ccncn1]

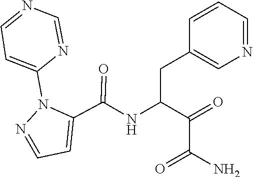 [SMILES: NC(=O)C(=O)C(Cc1cccnc1)NC(=O)c1ccnn1-c1ccncn1]

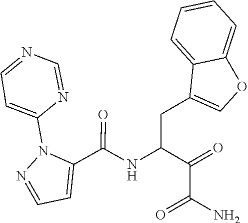 [SMILES: NC(=O)C(=O)C(Cc1coc2ccccc12)NC(=O)c1ccnn1-c1ccncn1]

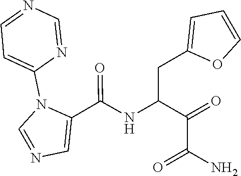 [SMILES: NC(=O)C(=O)C(Cc1ccco1)NC(=O)c1cncn1-c1ccncn1]

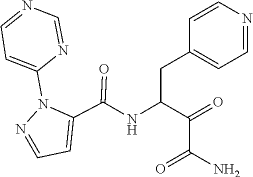 [SMILES: NC(=O)C(=O)C(Cc1ccncc1)NC(=O)c1ccnn1-c1ccncn1]

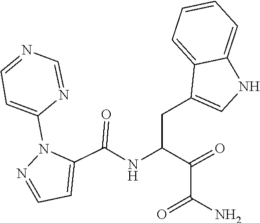 [SMILES: NC(=O)C(=O)C(Cc1c[nH]c2ccccc12)NC(=O)c1ccnn1-c1ccncn1]

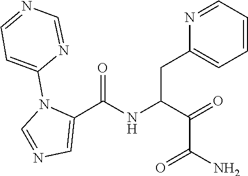 [SMILES: NC(=O)C(=O)C(Cc1ccccn1)NC(=O)c1cncn1-c1ccncn1]

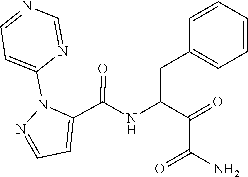 [SMILES: NC(=O)C(=O)C(Cc1ccccc1)NC(=O)c1ccnn1-c1ccncn1]

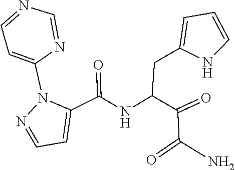 [SMILES: NC(=O)C(=O)C(Cc1ccc[nH]1)NC(=O)c1ccnn1-c1ccncn1]

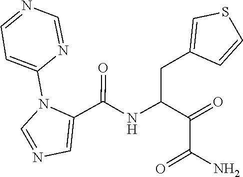 [SMILES: NC(=O)C(=O)C(Cc1ccsc1)NC(=O)c1cncn1-c1ccncn1]

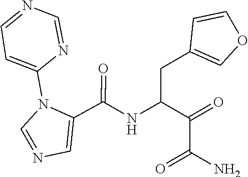 [SMILES: NC(=O)C(=O)C(Cc1ccoc1)NC(=O)c1cncn1-c1ccncn1]

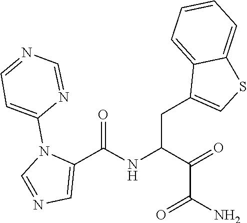 [SMILES: NC(=O)C(=O)C(Cc1csc2ccccc12)NC(=O)c1cncn1-c1ccncn1]

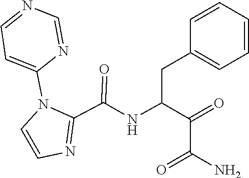 [SMILES: NC(=O)C(=O)C(Cc1ccccc1)NC(=O)c1nccn1-c1ccncn1]

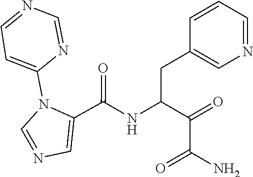 [SMILES: NC(=O)C(=O)C(Cc1cccnc1)NC(=O)c1cncn1-c1ccncn1]

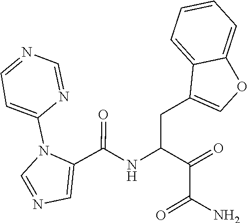 [SMILES: NC(=O)C(=O)C(Cc1coc2ccccc12)NC(=O)c1cncn1-c1ccncn1]

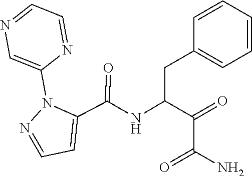 [SMILES: NC(=O)C(=O)C(Cc1ccccc1)NC(=O)c1ccnn1-c1cnccn1]

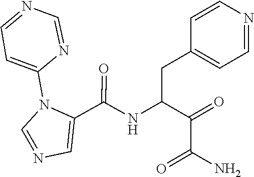 [SMILES: NC(=O)C(=O)C(Cc1ccncc1)NC(=O)c1cncn1-c1ccncn1]

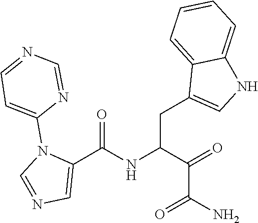 [SMILES: NC(=O)C(=O)C(Cc1c[nH]c2ccccc12)NC(=O)c1cncn1-c1ccncn1]

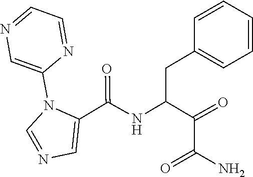 [SMILES: NC(=O)C(=O)C(Cc1ccccc1)NC(=O)c1cncn1-c1cnccn1]

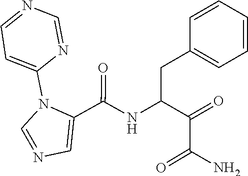 [SMILES: NC(=O)C(=O)C(Cc1ccccc1)NC(=O)c1cncn1-c1ccncn1]

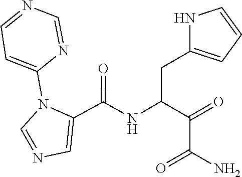 [SMILES: NC(=O)C(=O)C(Cc1ccc[nH]1)NC(=O)c1cncn1-c1ccncn1]

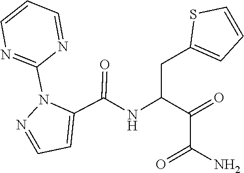 [SMILES: NC(=O)C(=O)C(Cc1cccs1)NC(=O)c1ccnn1-c1ncccn1]

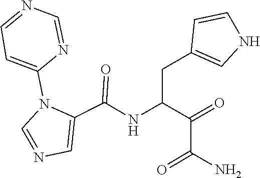 [SMILES: NC(=O)C(=O)C(Cc1cc[nH]c1)NC(=O)c1cncn1-c1ccncn1]

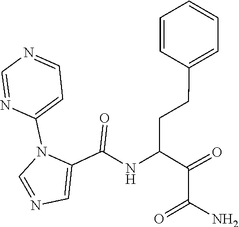 [SMILES: NC(=O)C(=O)C(CCc1ccccc1)NC(=O)c1cncn1-c1ccncn1]

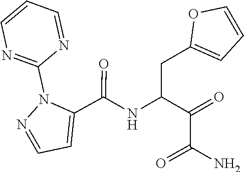 [SMILES: NC(=O)C(=O)C(Cc1ccco1)NC(=O)c1ccnn1-c1ncccn1]

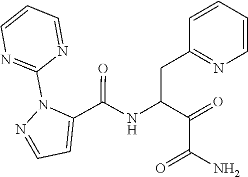 [SMILES: NC(=O)C(=O)C(Cc1ccccn1)NC(=O)c1ccnn1-c1ncccn1]

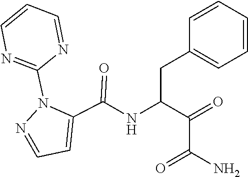 [SMILES: NC(=O)C(=O)C(Cc1ccccc1)NC(=O)c1ccnn1-c1ncccn1]

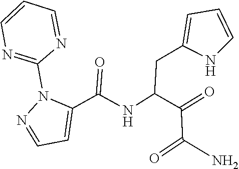 [SMILES: NC(=O)C(=O)C(Cc1ccc[nH]1)NC(=O)c1ccnn1-c1ncccn1]

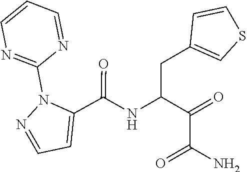 [SMILES: NC(=O)C(=O)C(Cc1ccsc1)NC(=O)c1ccnn1-c1ncccn1]

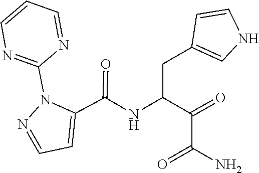 [SMILES: NC(=O)C(=O)C(Cc1cc[nH]c1)NC(=O)c1ccnn1-c1ncccn1]

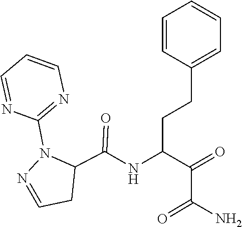 [SMILES: NC(=O)C(=O)C(CCc1ccccc1)NC(=O)C1CC=NN1c1ncccn1]

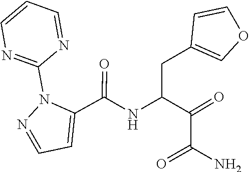 [SMILES: NC(=O)C(=O)C(Cc1ccoc1)NC(=O)c1ccnn1-c1ncccn1]

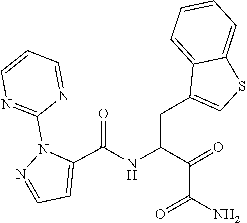 [SMILES: NC(=O)C(=O)C(Cc1csc2ccccc12)NC(=O)c1ccnn1-c1ncccn1]

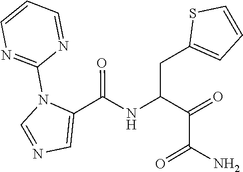 [SMILES: NC(=O)C(=O)C(Cc1cccs1)NC(=O)c1cncn1-c1ncccn1]

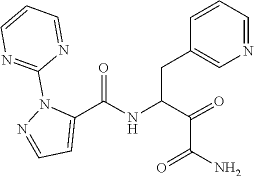 [SMILES: NC(=O)C(=O)C(Cc1cccnc1)NC(=O)c1ccnn1-c1ncccn1]

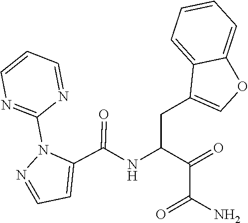 [SMILES: NC(=O)C(=O)C(Cc1coc2ccccc12)NC(=O)c1ccnn1-c1ncccn1]

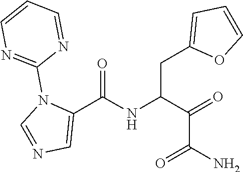 [SMILES: NC(=O)C(=O)C(Cc1ccco1)NC(=O)c1cncn1-c1ncccn1]

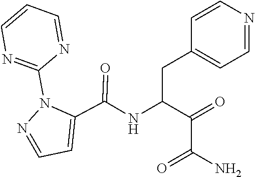 [SMILES: NC(=O)C(=O)C(Cc1ccncc1)NC(=O)c1ccnn1-c1ncccn1]

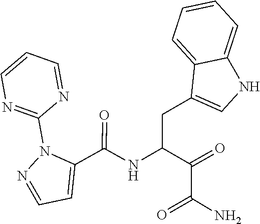 [SMILES: NC(=O)C(=O)C(Cc1c[nH]c2ccccc12)NC(=O)c1ccnn1-c1ncccn1]

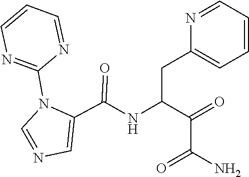 [SMILES: NC(=O)C(=O)C(Cc1ccccn1)NC(=O)c1cncn1-c1ncccn1]

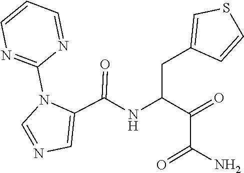 [SMILES: NC(=O)C(=O)C(Cc1ccsc1)NC(=O)c1cncn1-c1ncccn1]

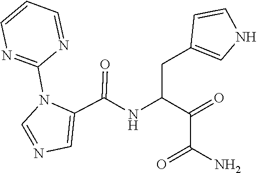 [SMILES: NC(=O)C(=O)C(Cc1cc[nH]c1)NC(=O)c1cncn1-c1ncccn1]

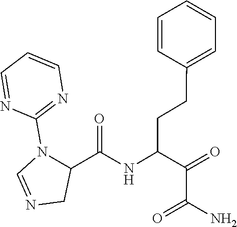 [SMILES: NC(=O)C(=O)C(CCc1ccccc1)NC(=O)C1CN=CN1c1ncccn1]

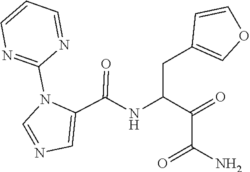 [SMILES: NC(=O)C(=O)C(Cc1ccoc1)NC(=O)c1cncn1-c1ncccn1]

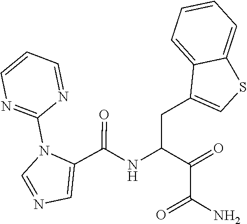 [SMILES: NC(=O)C(=O)C(Cc1csc2ccccc12)NC(=O)c1cncn1-c1ncccn1]

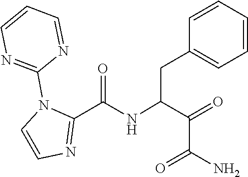 [SMILES: NC(=O)C(=O)C(Cc1ccccc1)NC(=O)c1nccn1-c1ncccn1]

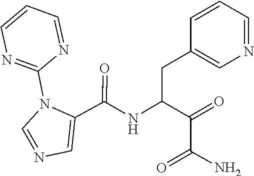 [SMILES: NC(=O)C(=O)C(Cc1cccnc1)NC(=O)c1cncn1-c1ncccn1]

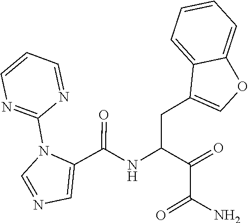 [SMILES: NC(=O)C(=O)C(Cc1coc2ccccc12)NC(=O)c1cncn1-c1ncccn1]

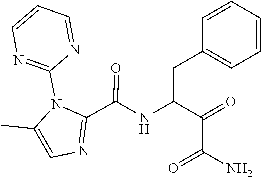 [SMILES: Cc1cnc(C(=O)NC(Cc2ccccc2)C(=O)C(N)=O)n1-c1ncccn1]

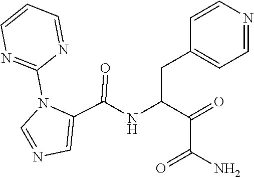 [SMILES: NC(=O)C(=O)C(Cc1ccncc1)NC(=O)c1cncn1-c1ncccn1]

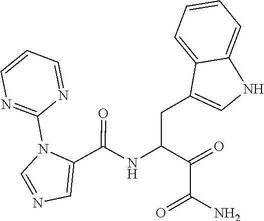 [SMILES: NC(=O)C(=O)C(Cc1c[nH]c2ccccc12)NC(=O)c1cncn1-c1ncccn1]

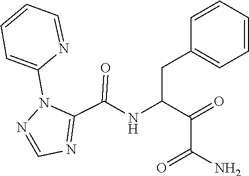 [SMILES: NC(=O)C(=O)C(Cc1ccccc1)NC(=O)c1ncnn1-c1ccccn1]

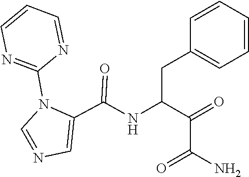 [SMILES: NC(=O)C(=O)C(Cc1ccccc1)NC(=O)c1cncn1-c1ncccn1]

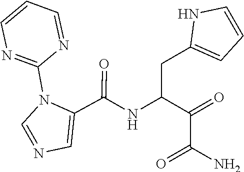 [SMILES: NC(=O)C(=O)C(Cc1ccc[nH]1)NC(=O)c1cncn1-c1ncccn1]

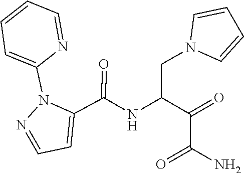 [SMILES: NC(=O)C(=O)C(Cn1cccc1)NC(=O)c1ccnn1-c1ccccn1]

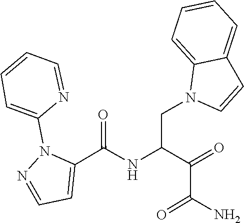 [SMILES: NC(=O)C(=O)C(Cn1ccc2ccccc21)NC(=O)c1ccnn1-c1ccccn1]

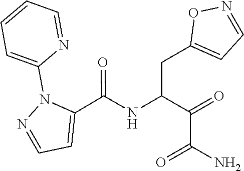 [SMILES: NC(=O)C(=O)C(Cc1ccno1)NC(=O)c1ccnn1-c1ccccn1]

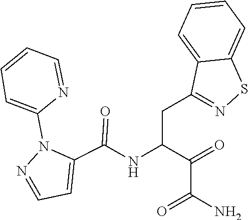 [SMILES: NC(=O)C(=O)C(Cc1nsc2ccccc12)NC(=O)c1ccnn1-c1ccccn1]

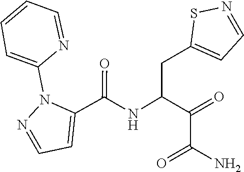 [SMILES: NC(=O)C(=O)C(Cc1ccns1)NC(=O)c1ccnn1-c1ccccn1]

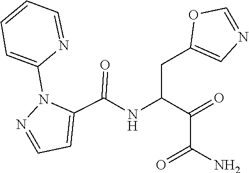 [SMILES: NC(=O)C(=O)C(Cc1cnco1)NC(=O)c1ccnn1-c1ccccn1]

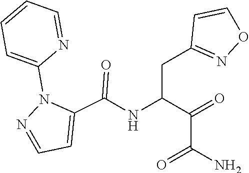 [SMILES: NC(=O)C(=O)C(Cc1ccon1)NC(=O)c1ccnn1-c1ccccn1]

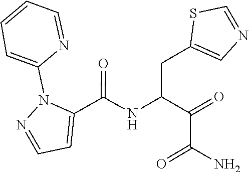 [SMILES: NC(=O)C(=O)C(Cc1cncs1)NC(=O)c1ccnn1-c1ccccn1]

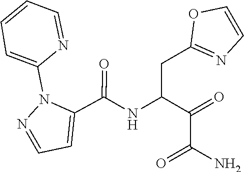 [SMILES: NC(=O)C(=O)C(Cc1ncco1)NC(=O)c1ccnn1-c1ccccn1]

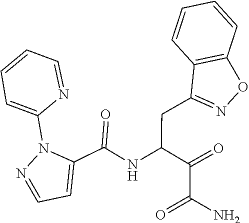 [SMILES: NC(=O)C(=O)C(Cc1noc2ccccc12)NC(=O)c1ccnn1-c1ccccn1]

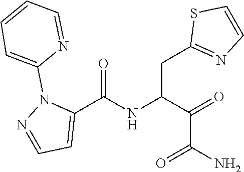 [SMILES: NC(=O)C(=O)C(Cc1nccs1)NC(=O)c1ccnn1-c1ccccn1]

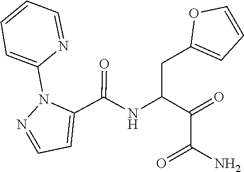 [SMILES: NC(=O)C(=O)C(Cc1ccco1)NC(=O)c1ccnn1-c1ccccn1]

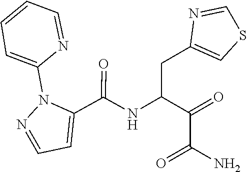 [SMILES: NC(=O)C(=O)C(Cc1cscn1)NC(=O)c1ccnn1-c1ccccn1]

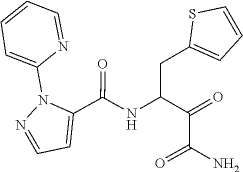 [SMILES: NC(=O)C(=O)C(Cc1cccs1)NC(=O)c1ccnn1-c1ccccn1]

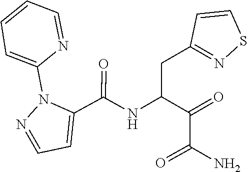 [SMILES: NC(=O)C(=O)C(Cc1ccsn1)NC(=O)c1ccnn1-c1ccccn1]

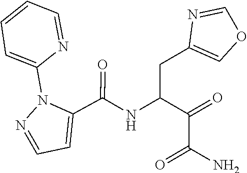 [SMILES: NC(=O)C(=O)C(Cc1cocn1)NC(=O)c1ccnn1-c1ccccn1]

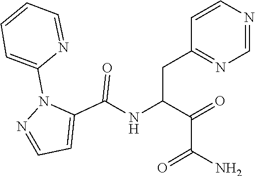 [SMILES: NC(=O)C(=O)C(Cc1ccncn1)NC(=O)c1ccnn1-c1ccccn1]

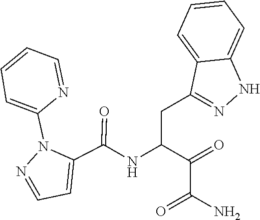 [SMILES: NC(=O)C(=O)C(Cc1n[nH]c2ccccc12)NC(=O)c1ccnn1-c1ccccn1]

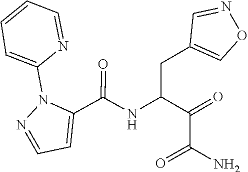 [SMILES: NC(=O)C(=O)C(Cc1cnoc1)NC(=O)c1ccnn1-c1ccccn1]

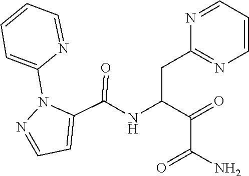 [SMILES: NC(=O)C(=O)C(Cc1ncccn1)NC(=O)c1ccnn1-c1ccccn1]

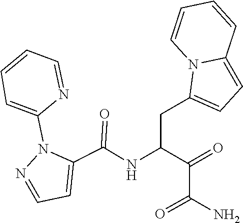 [SMILES: NC(=O)C(=O)C(Cc1ccc2ccccn12)NC(=O)c1ccnn1-c1ccccn1]

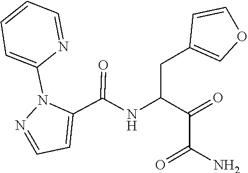 [SMILES: NC(=O)C(=O)C(Cc1ccoc1)NC(=O)c1ccnn1-c1ccccn1]

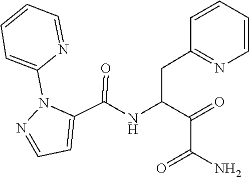 [SMILES: NC(=O)C(=O)C(Cc1ccccn1)NC(=O)c1ccnn1-c1ccccn1]

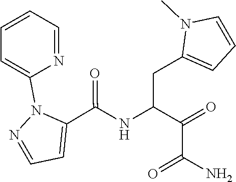 [SMILES: Cn1cccc1CC(NC(=O)c1ccnn1-c1ccccn1)C(=O)C(N)=O]

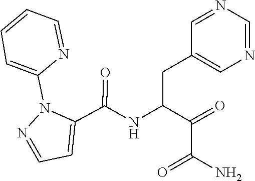 [SMILES: NC(=O)C(=O)C(Cc1cncnc1)NC(=O)c1ccnn1-c1ccccn1]

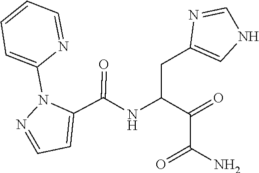 [SMILES: NC(=O)C(=O)C(Cc1c[nH]cn1)NC(=O)c1ccnn1-c1ccccn1]

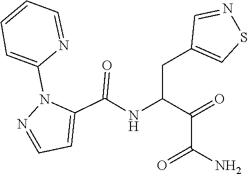 [SMILES: NC(=O)C(=O)C(Cc1cnsc1)NC(=O)c1ccnn1-c1ccccn1]

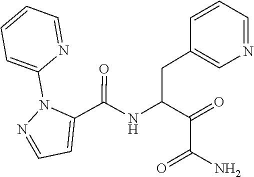 [SMILES: NC(=O)C(=O)C(Cc1cccnc1)NC(=O)c1ccnn1-c1ccccn1]

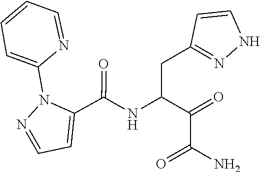 [SMILES: NC(=O)C(=O)C(Cc1cc[nH]n1)NC(=O)c1ccnn1-c1ccccn1]

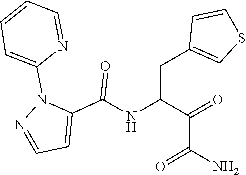 [SMILES: NC(=O)C(=O)C(Cc1ccsc1)NC(=O)c1ccnn1-c1ccccn1]

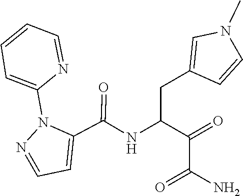 [SMILES: Cn1ccc(CC(NC(=O)c2ccnn2-c2ccccn2)C(=O)C(N)=O)c1]

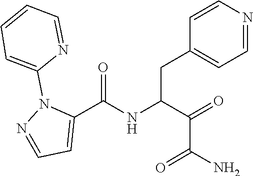 [SMILES: NC(=O)C(=O)C(Cc1ccncc1)NC(=O)c1ccnn1-c1ccccn1]

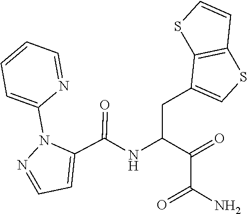 [SMILES: NC(=O)C(=O)C(Cc1csc2ccsc12)NC(=O)c1ccnn1-c1ccccn1]

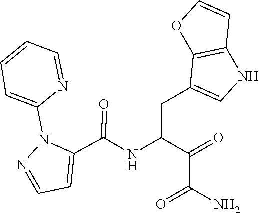 [SMILES: NC(=O)C(=O)C(Cc1c[nH]c2ccoc12)NC(=O)c1ccnn1-c1ccccn1]

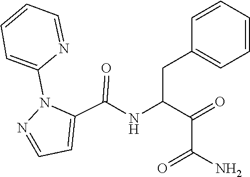 [SMILES: NC(=O)C(=O)C(Cc1ccccc1)NC(=O)c1ccnn1-c1ccccn1]

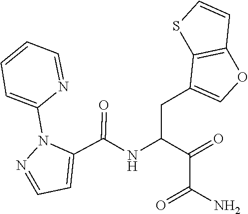 [SMILES: NC(=O)C(=O)C(Cc1coc2ccsc12)NC(=O)c1ccnn1-c1ccccn1]

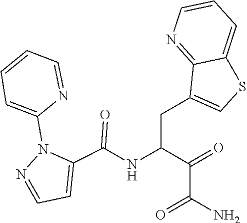 [SMILES: NC(=O)C(=O)C(Cc1csc2cccnc12)NC(=O)c1ccnn1-c1ccccn1]

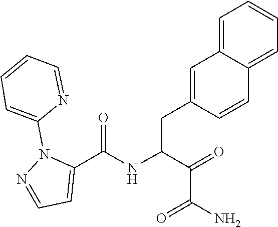 [SMILES: NC(=O)C(=O)C(Cc1ccc2ccccc2c1)NC(=O)c1ccnn1-c1ccccn1]

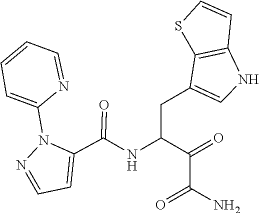 [SMILES: NC(=O)C(=O)C(Cc1c[nH]c2ccsc12)NC(=O)c1ccnn1-c1ccccn1]

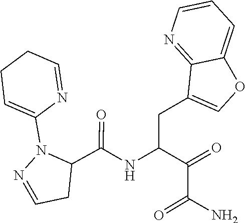 [SMILES: NC(=O)C(=O)C(Cc1coc2cccnc12)NC(=O)C1CC=NN1C1=CCCC=N1]

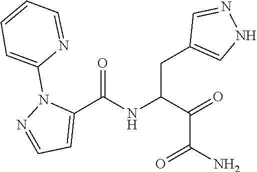 [SMILES: NC(=O)C(=O)C(Cc1cn[nH]c1)NC(=O)c1ccnn1-c1ccccn1]

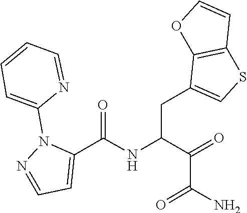 [SMILES: NC(=O)C(=O)C(Cc1csc2ccoc12)NC(=O)c1ccnn1-c1ccccn1]

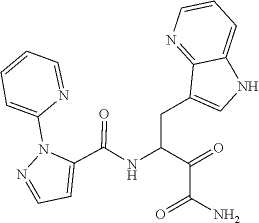 [SMILES: NC(=O)C(=O)C(Cc1c[nH]c2cccnc12)NC(=O)c1ccnn1-c1ccccn1]

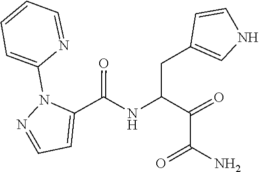 [SMILES: NC(=O)C(=O)C(Cc1cc[nH]c1)NC(=O)c1ccnn1-c1ccccn1]

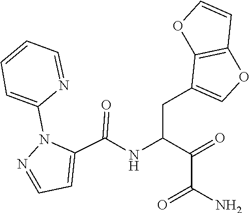 [SMILES: NC(=O)C(=O)C(Cc1coc2ccoc12)NC(=O)c1ccnn1-c1ccccn1]

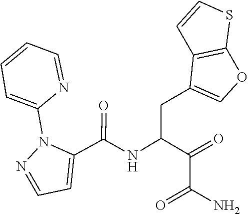 [SMILES: NC(=O)C(=O)C(Cc1coc2sccc12)NC(=O)c1ccnn1-c1ccccn1]

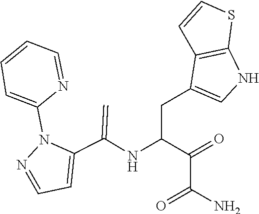 [SMILES: C=C(NC(Cc1c[nH]c2sccc12)C(=O)C(N)=O)c1ccnn1-c1ccccn1]

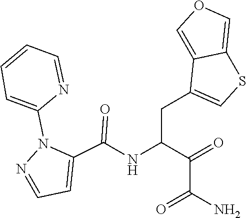 [SMILES: NC(=O)C(=O)C(Cc1csc2cocc12)NC(=O)c1ccnn1-c1ccccn1]

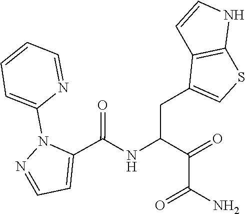 [SMILES: NC(=O)C(=O)C(Cc1csc2[nH]ccc12)NC(=O)c1ccnn1-c1ccccn1]

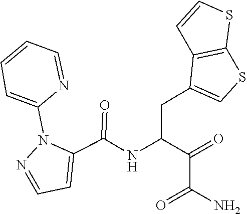 [SMILES: NC(=O)C(=O)C(Cc1csc2sccc12)NC(=O)c1ccnn1-c1ccccn1]

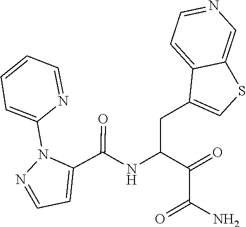 [SMILES: NC(=O)C(=O)C(Cc1csc2cnccc12)NC(=O)c1ccnn1-c1ccccn1]

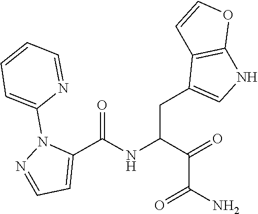 [SMILES: NC(=O)C(=O)C(Cc1c[nH]c2occc12)NC(=O)c1ccnn1-c1ccccn1]

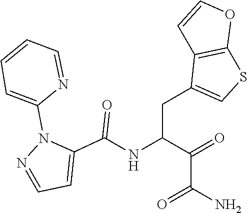 [SMILES: NC(=O)C(=O)C(Cc1csc2occc12)NC(=O)c1ccnn1-c1ccccn1]

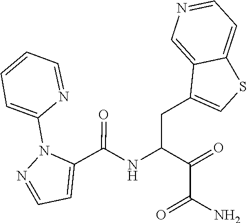 [SMILES: NC(=O)C(=O)C(Cc1csc2ccncc12)NC(=O)c1ccnn1-c1ccccn1]

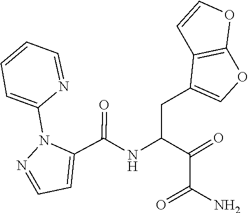 [SMILES: NC(=O)C(=O)C(Cc1coc2occc12)NC(=O)c1ccnn1-c1ccccn1]

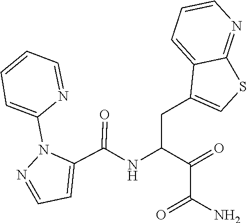 [SMILES: NC(=O)C(=O)C(Cc1csc2ncccc12)NC(=O)c1ccnn1-c1ccccn1]

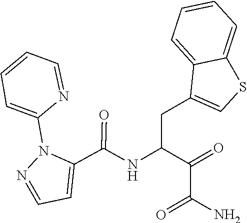 [SMILES: NC(=O)C(=O)C(Cc1csc2ccccc12)NC(=O)c1ccnn1-c1ccccn1]

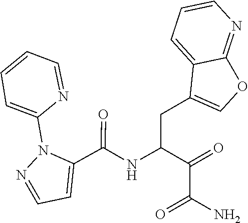 [SMILES: NC(=O)C(=O)C(Cc1coc2ncccc12)NC(=O)c1ccnn1-c1ccccn1]

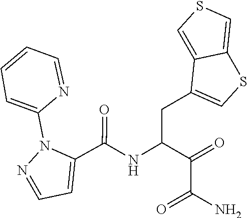 [SMILES: NC(=O)C(=O)C(Cc1csc2cscc12)NC(=O)c1ccnn1-c1ccccn1]

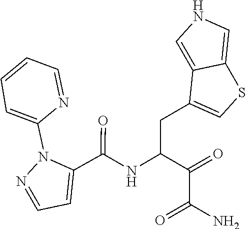 [SMILES: NC(=O)C(=O)C(Cc1csc2c[nH]cc12)NC(=O)c1ccnn1-c1ccccn1]

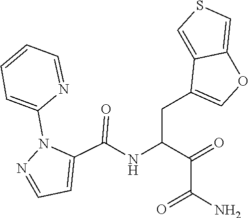 [SMILES: NC(=O)C(=O)C(Cc1coc2cscc12)NC(=O)c1ccnn1-c1ccccn1]

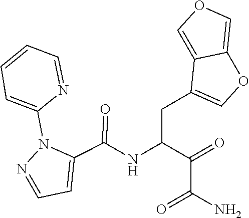 [SMILES: NC(=O)C(=O)C(Cc1coc2cocc12)NC(=O)c1ccnn1-c1ccccn1]

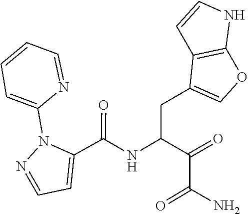 [SMILES: NC(=O)C(=O)C(Cc1coc2[nH]ccc12)NC(=O)c1ccnn1-c1ccccn1]

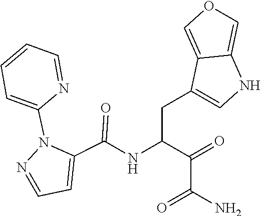 [SMILES: NC(=O)C(=O)C(Cc1c[nH]c2cocc12)NC(=O)c1ccnn1-c1ccccn1]

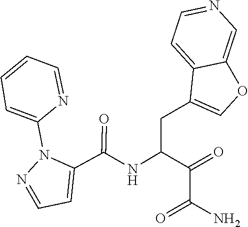 [SMILES: NC(=O)C(=O)C(Cc1coc2cnccc12)NC(=O)c1ccnn1-c1ccccn1]

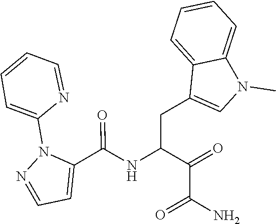 [SMILES: Cn1cc(CC(NC(=O)c2ccnn2-c2ccccn2)C(=O)C(N)=O)c2ccccc21]

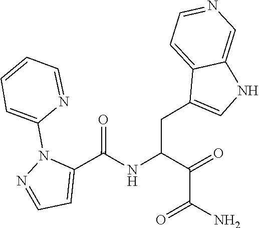 [SMILES: NC(=O)C(=O)C(Cc1c[nH]c2cnccc12)NC(=O)c1ccnn1-c1ccccn1]

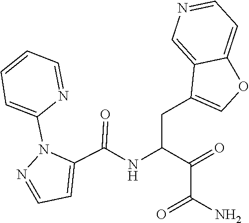 [SMILES: NC(=O)C(=O)C(Cc1coc2ccncc12)NC(=O)c1ccnn1-c1ccccn1]

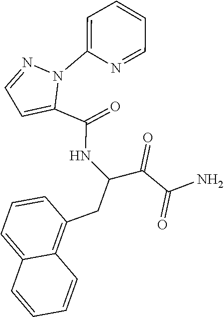 [SMILES: NC(=O)C(=O)C(Cc1cccc2ccccc12)NC(=O)c1ccnn1-c1ccccn1]

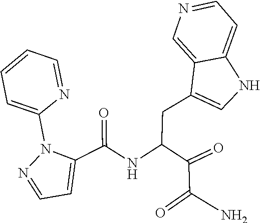 [SMILES: NC(=O)C(=O)C(Cc1c[nH]c2ccncc12)NC(=O)c1ccnn1-c1ccccn1]

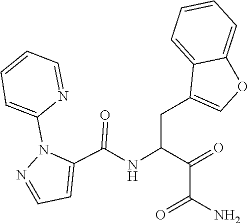 [SMILES: NC(=O)C(=O)C(Cc1coc2ccccc12)NC(=O)c1ccnn1-c1ccccn1]

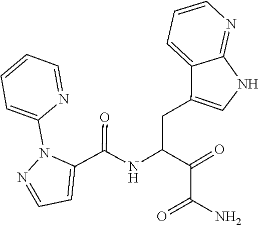 [SMILES: NC(=O)C(=O)C(Cc1c[nH]c2ncccc12)NC(=O)c1ccnn1-c1ccccn1]

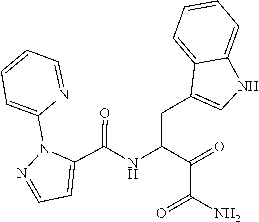 [SMILES: NC(=O)C(=O)C(Cc1c[nH]c2ccccc12)NC(=O)c1ccnn1-c1ccccn1]

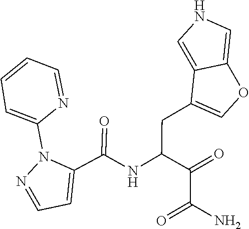 [SMILES: NC(=O)C(=O)C(Cc1coc2c[nH]cc12)NC(=O)c1ccnn1-c1ccccn1]

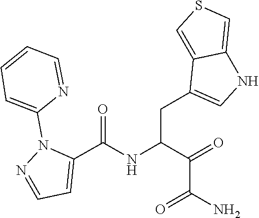 [SMILES: NC(=O)C(=O)C(Cc1c[nH]c2cscc12)NC(=O)c1ccnn1-c1ccccn1]

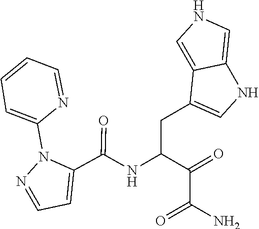 [SMILES: NC(=O)C(=O)C(Cc1c[nH]c2c[nH]cc12)NC(=O)c1ccnn1-c1ccccn1]

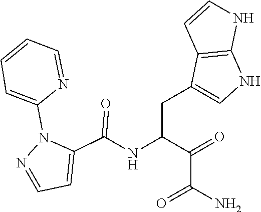 [SMILES: NC(=O)C(=O)C(Cc1c[nH]c2[nH]ccc12)NC(=O)c1ccnn1-c1ccccn1]

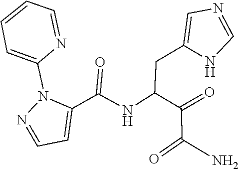 [SMILES: NC(=O)C(=O)C(Cc1cnc[nH]1)NC(=O)c1ccnn1-c1ccccn1]

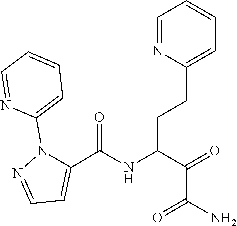 [SMILES: NC(=O)C(=O)C(CCc1ccccn1)NC(=O)c1ccnn1-c1ccccn1]

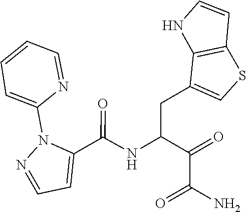 [SMILES: NC(=O)C(=O)C(Cc1csc2cc[nH]c12)NC(=O)c1ccnn1-c1ccccn1]

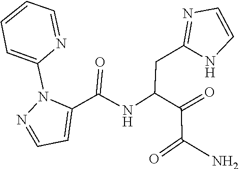 [SMILES: NC(=O)C(=O)C(Cc1ncc[nH]1)NC(=O)c1ccnn1-c1ccccn1]

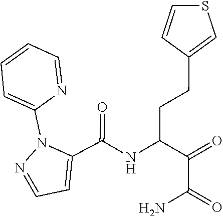 [SMILES: NC(=O)C(=O)C(CCc1ccsc1)NC(=O)c1ccnn1-c1ccccn1]

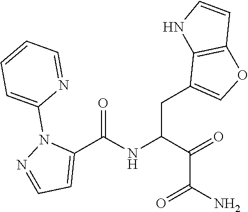 [SMILES: NC(=O)C(=O)C(Cc1coc2cc[nH]c12)NC(=O)c1ccnn1-c1ccccn1]

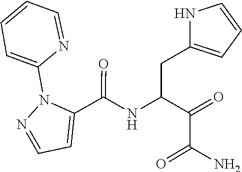 [SMILES: NC(=O)C(=O)C(Cc1ccc[nH]1)NC(=O)c1ccnn1-c1ccccn1]

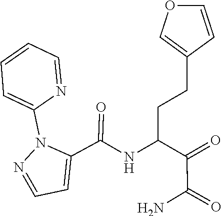 [SMILES: NC(=O)C(=O)C(CCc1ccoc1)NC(=O)c1ccnn1-c1ccccn1]

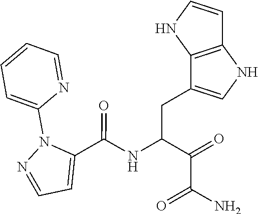 [SMILES: NC(=O)C(=O)C(Cc1c[nH]c2cc[nH]c12)NC(=O)c1ccnn1-c1ccccn1]

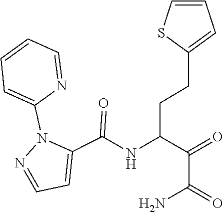 [SMILES: NC(=O)C(=O)C(CCc1cccs1)NC(=O)c1ccnn1-c1ccccn1]

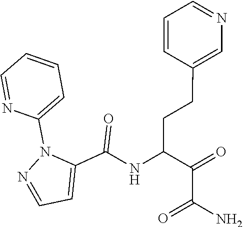 [SMILES: NC(=O)C(=O)C(CCc1cccnc1)NC(=O)c1ccnn1-c1ccccn1]

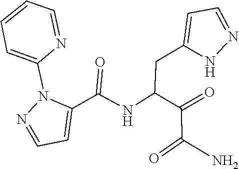 [SMILES: NC(=O)C(=O)C(Cc1ccn[nH]1)NC(=O)c1ccnn1-c1ccccn1]

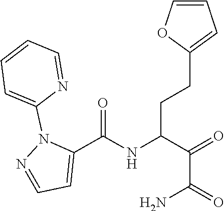 [SMILES: NC(=O)C(=O)C(CCc1ccco1)NC(=O)c1ccnn1-c1ccccn1]

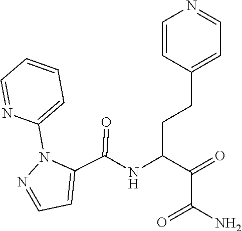 [SMILES: NC(=O)C(=O)C(CCc1ccncc1)NC(=O)c1ccnn1-c1ccccn1]

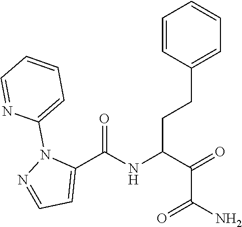 [SMILES: NC(=O)C(=O)C(CCc1ccccc1)NC(=O)c1ccnn1-c1ccccn1]

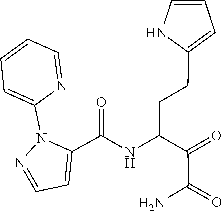 [SMILES: NC(=O)C(=O)C(CCc1ccc[nH]1)NC(=O)c1ccnn1-c1ccccn1]

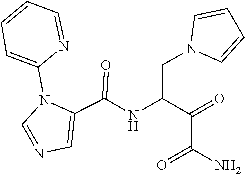 [SMILES: NC(=O)C(=O)C(Cn1cccc1)NC(=O)c1cncn1-c1ccccn1]

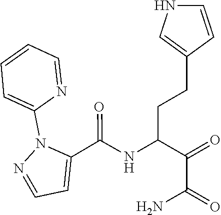 [SMILES: NC(=O)C(=O)C(CCc1cc[nH]c1)NC(=O)c1ccnn1-c1ccccn1]

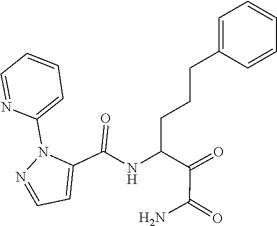 [SMILES: NC(=O)C(=O)C(CCCc1ccccc1)NC(=O)c1ccnn1-c1ccccn1]

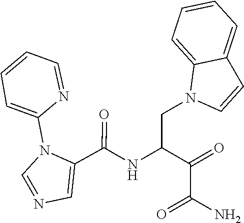 [SMILES: NC(=O)C(=O)C(Cn1ccc2ccccc21)NC(=O)c1cncn1-c1ccccn1]

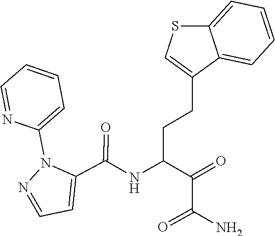 [SMILES: NC(=O)C(=O)C(CCc1csc2ccccc12)NC(=O)c1ccnn1-c1ccccn1]

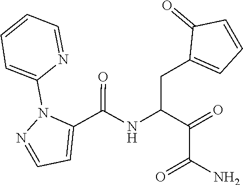 [SMILES: NC(=O)C(=O)C(CC1=CC=CC1=O)NC(=O)c1ccnn1-c1ccccn1]

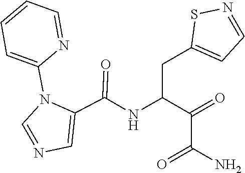 [SMILES: NC(=O)C(=O)C(Cc1ccns1)NC(=O)c1cncn1-c1ccccn1]

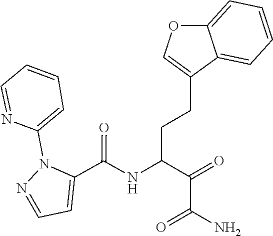 [SMILES: NC(=O)C(=O)C(CCc1coc2ccccc12)NC(=O)c1ccnn1-c1ccccn1]

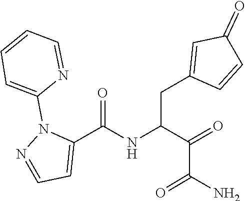 [SMILES: NC(=O)C(=O)C(CC1=CC(=O)C=C1)NC(=O)c1ccnn1-c1ccccn1]

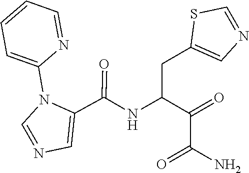 [SMILES: NC(=O)C(=O)C(Cc1cncs1)NC(=O)c1cncn1-c1ccccn1]

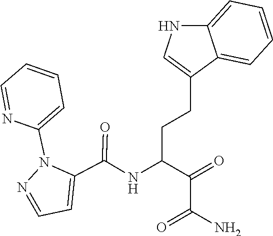 [SMILES: NC(=O)C(=O)C(CCc1c[nH]c2ccccc12)NC(=O)c1ccnn1-c1ccccn1]

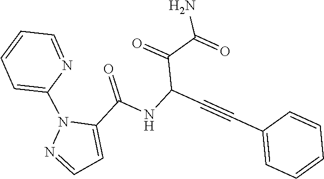 [SMILES: NC(=O)C(=O)C(C#Cc1ccccc1)NC(=O)c1ccnn1-c1ccccn1]

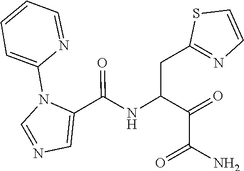 [SMILES: NC(=O)C(=O)C(Cc1nccs1)NC(=O)c1cncn1-c1ccccn1]

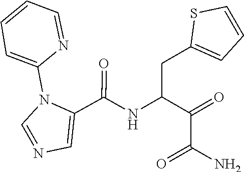 [SMILES: NC(=O)C(=O)C(Cc1cccs1)NC(=O)c1cncn1-c1ccccn1]

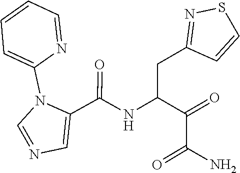 [SMILES: NC(=O)C(=O)C(Cc1ccsn1)NC(=O)c1cncn1-c1ccccn1]

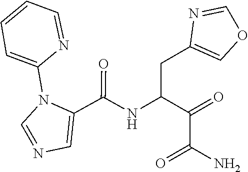 [SMILES: NC(=O)C(=O)C(Cc1cocn1)NC(=O)c1cncn1-c1ccccn1]

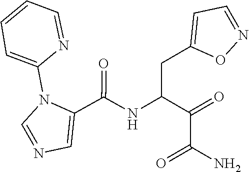 [SMILES: NC(=O)C(=O)C(Cc1ccno1)NC(=O)c1cncn1-c1ccccn1]

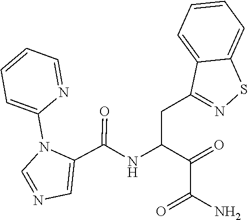 [SMILES: NC(=O)C(=O)C(Cc1nsc2ccccc12)NC(=O)c1cncn1-c1ccccn1]

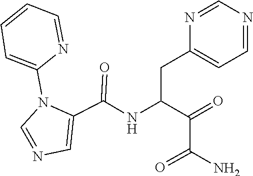 [SMILES: NC(=O)C(=O)C(Cc1ccncn1)NC(=O)c1cncn1-c1ccccn1]

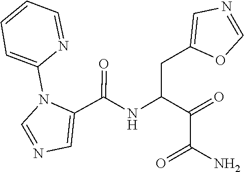 [SMILES: NC(=O)C(=O)C(Cc1cnco1)NC(=O)c1cncn1-c1ccccn1]

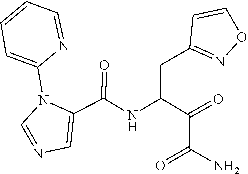 [SMILES: NC(=O)C(=O)C(Cc1ccon1)NC(=O)c1cncn1-c1ccccn1]

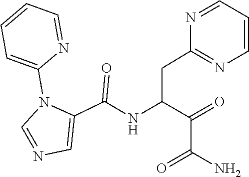 [SMILES: NC(=O)C(=O)C(Cc1ncccn1)NC(=O)c1cncn1-c1ccccn1]

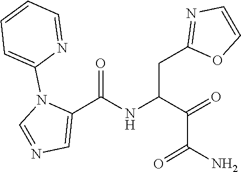 [SMILES: NC(=O)C(=O)C(Cc1ncco1)NC(=O)c1cncn1-c1ccccn1]

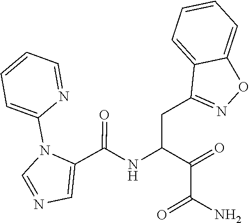 [SMILES: NC(=O)C(=O)C(Cc1noc2ccccc12)NC(=O)c1cncn1-c1ccccn1]

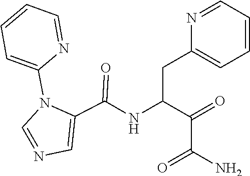 [SMILES: NC(=O)C(=O)C(Cc1ccccn1)NC(=O)c1cncn1-c1ccccn1]

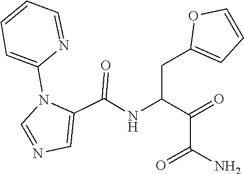 [SMILES: NC(=O)C(=O)C(Cc1ccco1)NC(=O)c1cncn1-c1ccccn1]

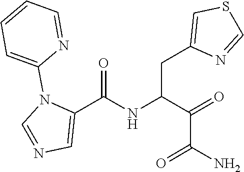 [SMILES: NC(=O)C(=O)C(Cc1cscn1)NC(=O)c1cncn1-c1ccccn1]

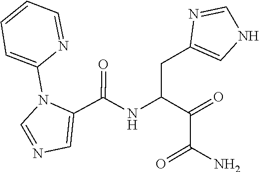 [SMILES: NC(=O)C(=O)C(Cc1c[nH]cn1)NC(=O)c1cncn1-c1ccccn1]

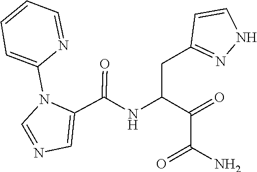 [SMILES: NC(=O)C(=O)C(Cc1cc[nH]n1)NC(=O)c1cncn1-c1ccccn1]

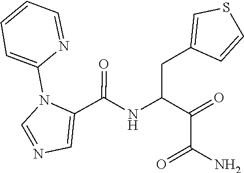 [SMILES: NC(=O)C(=O)C(Cc1ccsc1)NC(=O)c1cncn1-c1ccccn1]

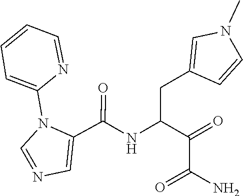 [SMILES: Cn1ccc(CC(NC(=O)c2cncn2-c2ccccn2)C(=O)C(N)=O)c1]

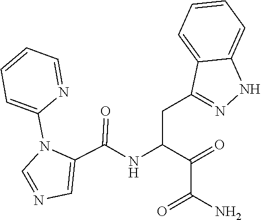 [SMILES: NC(=O)C(=O)C(Cc1n[nH]c2ccccc12)NC(=O)c1cncn1-c1ccccn1]

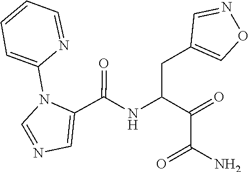 [SMILES: NC(=O)C(=O)C(Cc1cnoc1)NC(=O)c1cncn1-c1ccccn1]

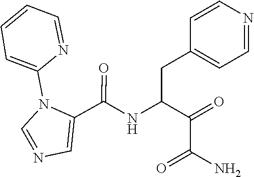 [SMILES: NC(=O)C(=O)C(Cc1ccncc1)NC(=O)c1cncn1-c1ccccn1]

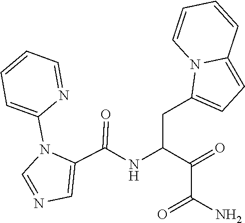 [SMILES: NC(=O)C(=O)C(Cc1ccc2ccccn12)NC(=O)c1cncn1-c1ccccn1]

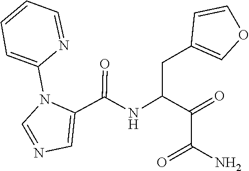 [SMILES: NC(=O)C(=O)C(Cc1ccoc1)NC(=O)c1cncn1-c1ccccn1]

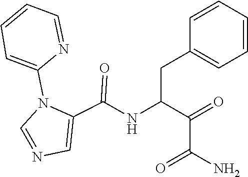 [SMILES: NC(=O)C(=O)C(Cc1ccccc1)NC(=O)c1cncn1-c1ccccn1]

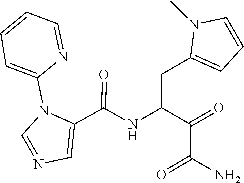 [SMILES: Cn1cccc1CC(NC(=O)c1cncn1-c1ccccn1)C(=O)C(N)=O]

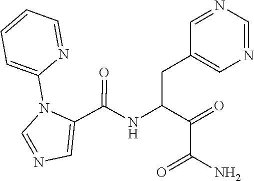 [SMILES: NC(=O)C(=O)C(Cc1cncnc1)NC(=O)c1cncn1-c1ccccn1]

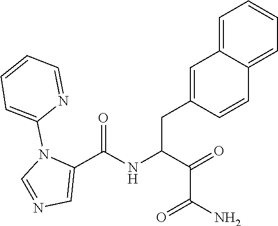 [SMILES: NC(=O)C(=O)C(Cc1ccc2ccccc2c1)NC(=O)c1cncn1-c1ccccn1]

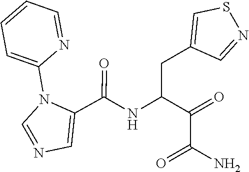 [SMILES: NC(=O)C(=O)C(Cc1cnsc1)NC(=O)c1cncn1-c1ccccn1]

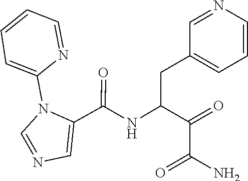 [SMILES: NC(=O)C(=O)C(Cc1cccnc1)NC(=O)c1cncn1-c1ccccn1]

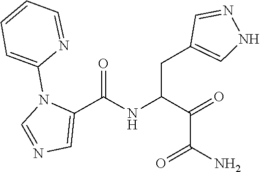 [SMILES: NC(=O)C(=O)C(Cc1cn[nH]c1)NC(=O)c1cncn1-c1ccccn1]

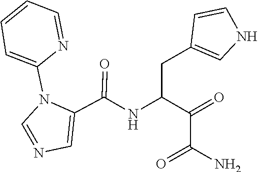 [SMILES: NC(=O)C(=O)C(Cc1cc[nH]c1)NC(=O)c1cncn1-c1ccccn1]

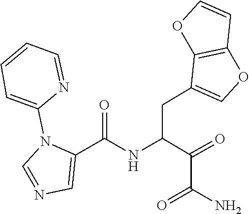 [SMILES: NC(=O)C(=O)C(Cc1coc2ccoc12)NC(=O)c1cncn1-c1ccccn1]

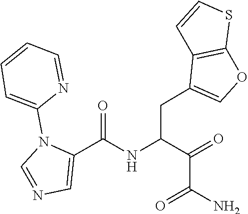 [SMILES: NC(=O)C(=O)C(Cc1coc2sccc12)NC(=O)c1cncn1-c1ccccn1]

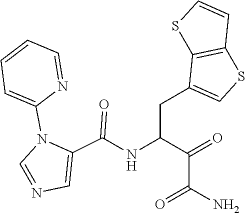 [SMILES: NC(=O)C(=O)C(Cc1csc2ccsc12)NC(=O)c1cncn1-c1ccccn1]

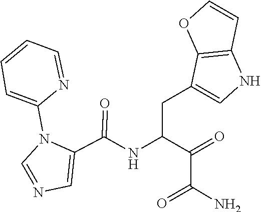 [SMILES: NC(=O)C(=O)C(Cc1c[nH]c2ccoc12)NC(=O)c1cncn1-c1ccccn1]

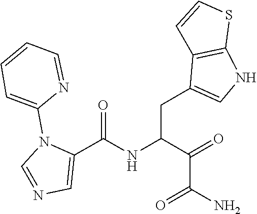 [SMILES: NC(=O)C(=O)C(Cc1c[nH]c2sccc12)NC(=O)c1cncn1-c1ccccn1]

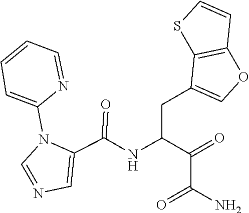 [SMILES: NC(=O)C(=O)C(Cc1coc2ccsc12)NC(=O)c1cncn1-c1ccccn1]

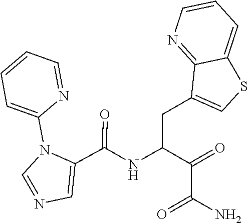 [SMILES: NC(=O)C(=O)C(Cc1csc2cccnc12)NC(=O)c1cncn1-c1ccccn1]

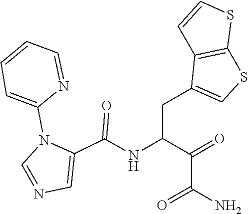 [SMILES: NC(=O)C(=O)C(Cc1csc2sccc12)NC(=O)c1cncn1-c1ccccn1]

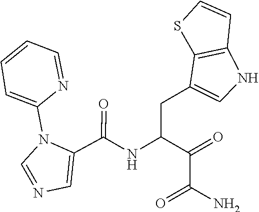 [SMILES: NC(=O)C(=O)C(Cc1c[nH]c2ccsc12)NC(=O)c1cncn1-c1ccccn1]

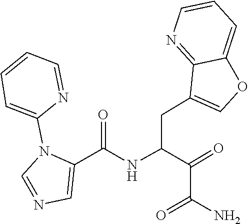 [SMILES: NC(=O)C(=O)C(Cc1coc2cccnc12)NC(=O)c1cncn1-c1ccccn1]

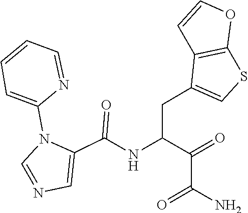 [SMILES: NC(=O)C(=O)C(Cc1csc2occc12)NC(=O)c1cncn1-c1ccccn1]

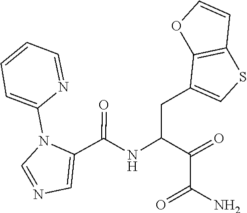 [SMILES: NC(=O)C(=O)C(Cc1csc2ccoc12)NC(=O)c1cncn1-c1ccccn1]

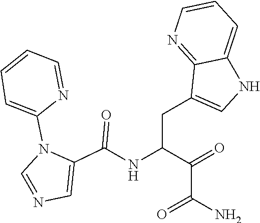 [SMILES: NC(=O)C(=O)C(Cc1c[nH]c2cccnc12)NC(=O)c1cncn1-c1ccccn1]

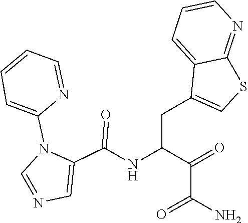 [SMILES: NC(=O)C(=O)C(Cc1csc2ncccc12)NC(=O)c1cncn1-c1ccccn1]

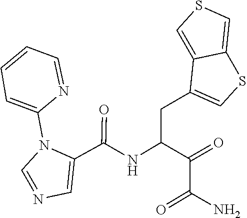 [SMILES: NC(=O)C(=O)C(Cc1csc2cscc12)NC(=O)c1cncn1-c1ccccn1]

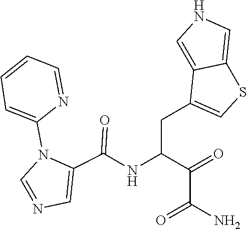 [SMILES: NC(=O)C(=O)C(Cc1csc2c[nH]cc12)NC(=O)c1cncn1-c1ccccn1]

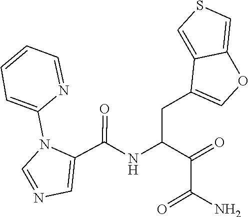 [SMILES: NC(=O)C(=O)C(Cc1coc2cscc12)NC(=O)c1cncn1-c1ccccn1]

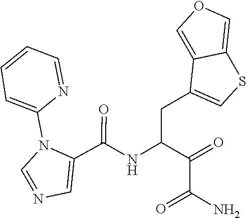 [SMILES: NC(=O)C(=O)C(Cc1csc2cocc12)NC(=O)c1cncn1-c1ccccn1]

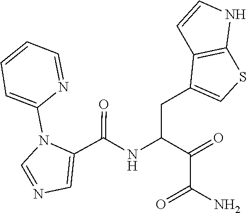 [SMILES: NC(=O)C(=O)C(Cc1csc2[nH]ccc12)NC(=O)c1cncn1-c1ccccn1]

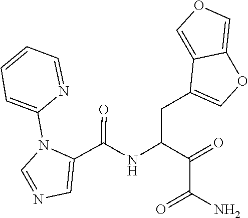 [SMILES: NC(=O)C(=O)C(Cc1coc2cocc12)NC(=O)c1cncn1-c1ccccn1]

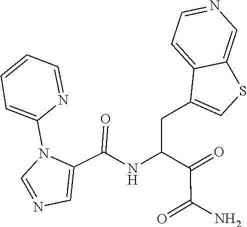 [SMILES: NC(=O)C(=O)C(Cc1csc2cnccc12)NC(=O)c1cncn1-c1ccccn1]

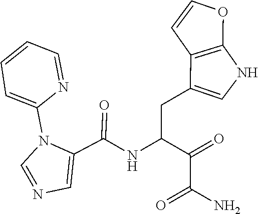 [SMILES: NC(=O)C(=O)C(Cc1c[nH]c2occc12)NC(=O)c1cncn1-c1ccccn1]

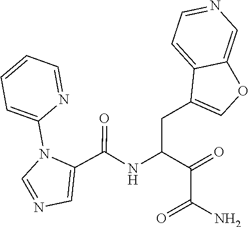 [SMILES: NC(=O)C(=O)C(Cc1coc2cnccc12)NC(=O)c1cncn1-c1ccccn1]

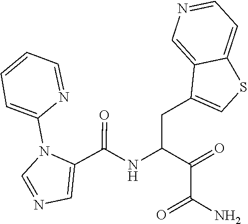 [SMILES: NC(=O)C(=O)C(Cc1csc2ccncc12)NC(=O)c1cncn1-c1ccccn1]

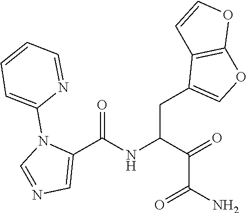 [SMILES: NC(=O)C(=O)C(Cc1coc2occc12)NC(=O)c1cncn1-c1ccccn1]

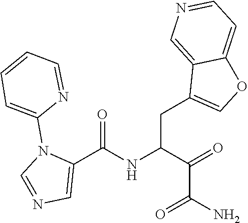 [SMILES: NC(=O)C(=O)C(Cc1coc2ccncc12)NC(=O)c1cncn1-c1ccccn1]

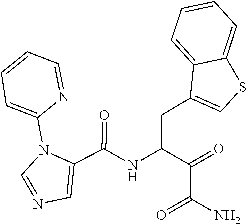 [SMILES: NC(=O)C(=O)C(Cc1csc2ccccc12)NC(=O)c1cncn1-c1ccccn1]

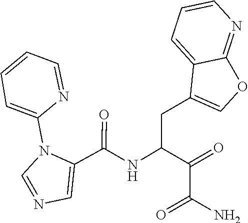 [SMILES: NC(=O)C(=O)C(Cc1coc2ncccc12)NC(=O)c1cncn1-c1ccccn1]

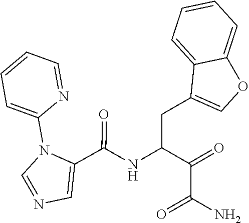 [SMILES: NC(=O)C(=O)C(Cc1coc2ccccc12)NC(=O)c1cncn1-c1ccccn1]

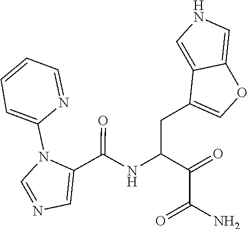 [SMILES: NC(=O)C(=O)C(Cc1coc2c[nH]cc12)NC(=O)c1cncn1-c1ccccn1]

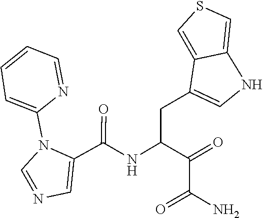 [SMILES: NC(=O)C(=O)C(Cc1c[nH]c2cscc12)NC(=O)c1cncn1-c1ccccn1]

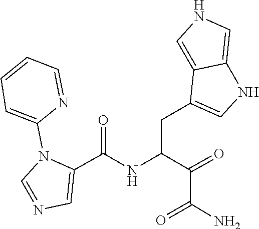 [SMILES: NC(=O)C(=O)C(Cc1c[nH]c2c[nH]cc12)NC(=O)c1cncn1-c1ccccn1]

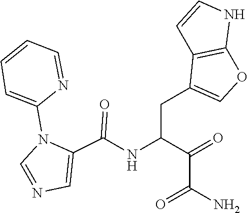 [SMILES: NC(=O)C(=O)C(Cc1coc2[nH]ccc12)NC(=O)c1cncn1-c1ccccn1]

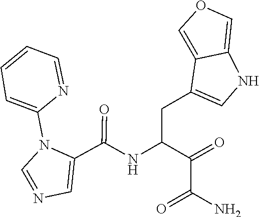 [SMILES: NC(=O)C(=O)C(Cc1c[nH]c2cocc12)NC(=O)c1cncn1-c1ccccn1]

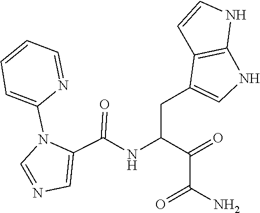 [SMILES: NC(=O)C(=O)C(Cc1c[nH]c2[nH]ccc12)NC(=O)c1cncn1-c1ccccn1]

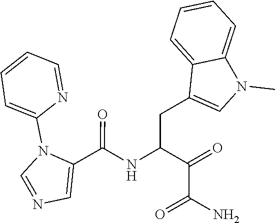 [SMILES: Cn1cc(CC(NC(=O)c2cncn2-c2ccccn2)C(=O)C(N)=O)c2ccccc21]

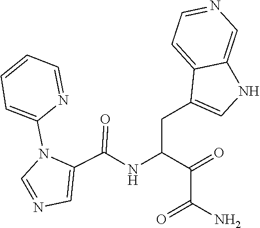 [SMILES: NC(=O)C(=O)C(Cc1c[nH]c2cnccc12)NC(=O)c1cncn1-c1ccccn1]

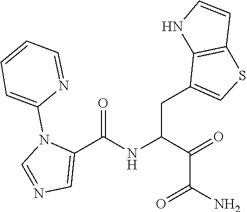 [SMILES: NC(=O)C(=O)C(Cc1csc2cc[nH]c12)NC(=O)c1cncn1-c1ccccn1]

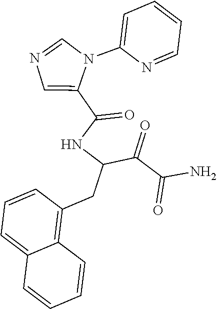 [SMILES: NC(=O)C(=O)C(Cc1cccc2ccccc12)NC(=O)c1cncn1-c1ccccn1]

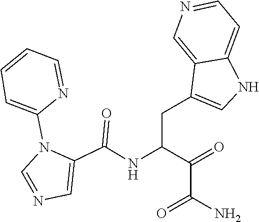 [SMILES: NC(=O)C(=O)C(Cc1c[nH]c2ccncc12)NC(=O)c1cncn1-c1ccccn1]

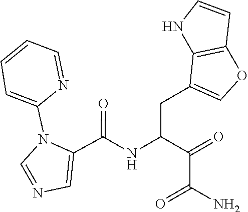 [SMILES: NC(=O)C(=O)C(Cc1coc2cc[nH]c12)NC(=O)c1cncn1-c1ccccn1]

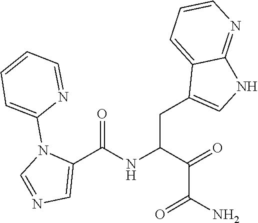 [SMILES: NC(=O)C(=O)C(Cc1c[nH]c2ncccc12)NC(=O)c1cncn1-c1ccccn1]

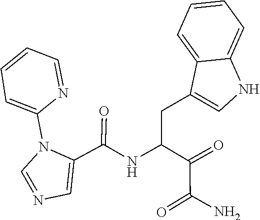 [SMILES: NC(=O)C(=O)C(Cc1c[nH]c2ccccc12)NC(=O)c1cncn1-c1ccccn1]

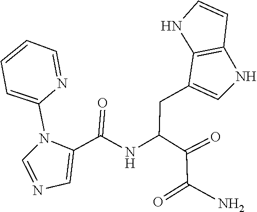 [SMILES: NC(=O)C(=O)C(Cc1c[nH]c2cc[nH]c12)NC(=O)c1cncn1-c1ccccn1]

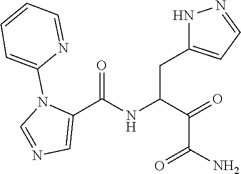 [SMILES: NC(=O)C(=O)C(Cc1ccn[nH]1)NC(=O)c1cncn1-c1ccccn1]

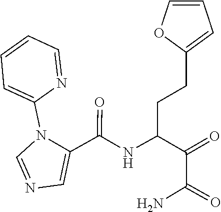 [SMILES: NC(=O)C(=O)C(CCc1ccco1)NC(=O)c1cncn1-c1ccccn1]

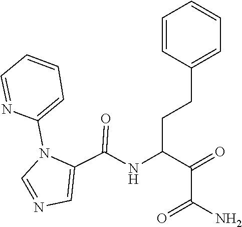 [SMILES: NC(=O)C(=O)C(CCc1ccccc1)NC(=O)c1cncn1-c1ccccn1]

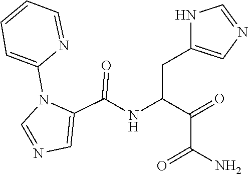 [SMILES: NC(=O)C(=O)C(Cc1cnc[nH]1)NC(=O)c1cncn1-c1ccccn1]

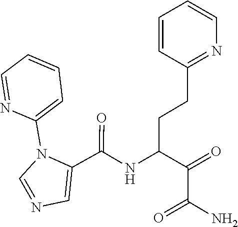 [SMILES: NC(=O)C(=O)C(CCc1ccccn1)NC(=O)c1cncn1-c1ccccn1]

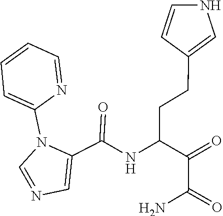 [SMILES: NC(=O)C(=O)C(CCc1cc[nH]c1)NC(=O)c1cncn1-c1ccccn1]

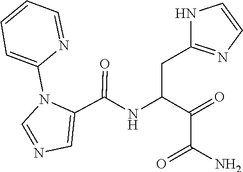 [SMILES: NC(=O)C(=O)C(Cc1ncc[nH]1)NC(=O)c1cncn1-c1ccccn1]

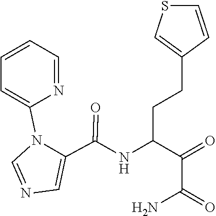 [SMILES: NC(=O)C(=O)C(CCc1ccsc1)NC(=O)c1cncn1-c1ccccn1]

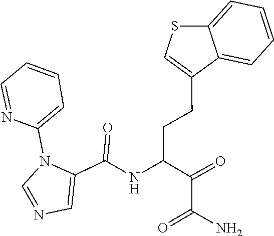 [SMILES: NC(=O)C(=O)C(CCc1csc2ccccc12)NC(=O)c1cncn1-c1ccccn1]

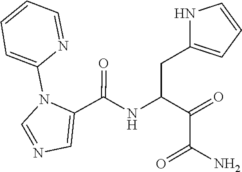 [SMILES: NC(=O)C(=O)C(Cc1ccc[nH]1)NC(=O)c1cncn1-c1ccccn1]

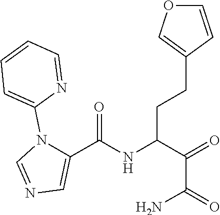 [SMILES: NC(=O)C(=O)C(CCc1ccoc1)NC(=O)c1cncn1-c1ccccn1]

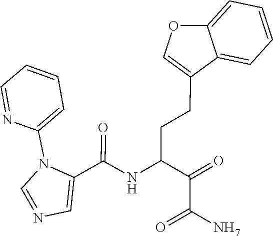

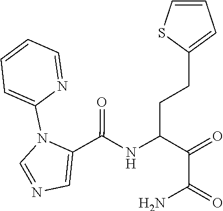 [SMILES: NC(=O)C(=O)C(CCc1cccs1)NC(=O)c1cncn1-c1ccccn1]

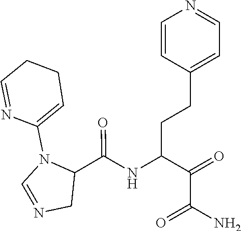 [SMILES: NC(=O)C(=O)C(CCc1ccncc1)NC(=O)C1CN=CN1C1=CCCC=N1]

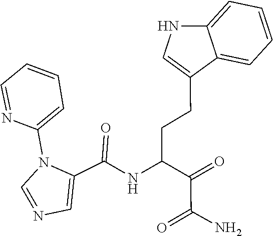 [SMILES: NC(=O)C(=O)C(CCc1c[nH]c2ccccc12)NC(=O)c1cncn1-c1ccccn1]

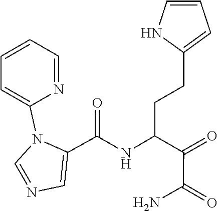 [SMILES: NC(=O)C(=O)C(CCc1ccc[nH]1)NC(=O)c1cncn1-c1ccccn1]

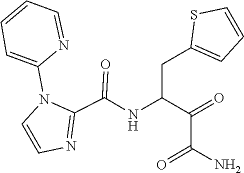 [SMILES: NC(=O)C(=O)C(Cc1cccs1)NC(=O)c1nccn1-c1ccccn1]

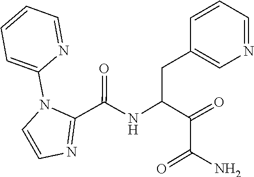 [SMILES: NC(=O)C(=O)C(Cc1cccnc1)NC(=O)c1nccn1-c1ccccn1]

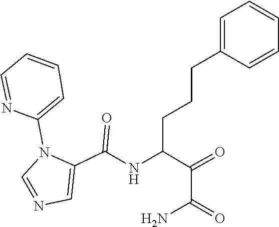 [SMILES: NC(=O)C(=O)C(CCCc1ccccc1)NC(=O)c1cncn1-c1ccccn1]

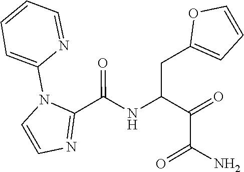 [SMILES: NC(=O)C(=O)C(Cc1ccco1)NC(=O)c1nccn1-c1ccccn1]

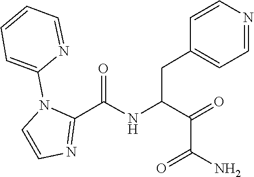 [SMILES: NC(=O)C(=O)C(Cc1ccncc1)NC(=O)c1nccn1-c1ccccn1]

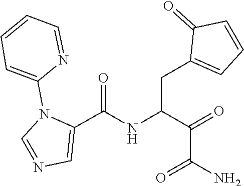 [SMILES: NC(=O)C(=O)C(CC1=CC=CC1=O)NC(=O)c1cncn1-c1ccccn1]

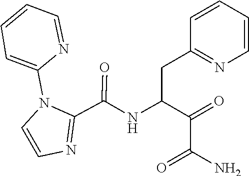 [SMILES: NC(=O)C(=O)C(Cc1ccccn1)NC(=O)c1nccn1-c1ccccn1]

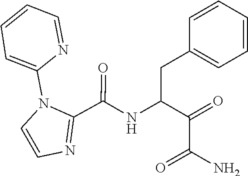 [SMILES: NC(=O)C(=O)C(Cc1ccccc1)NC(=O)c1nccn1-c1ccccn1]

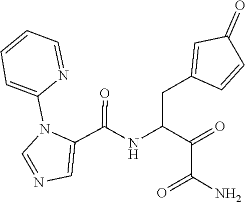 [SMILES: NC(=O)C(=O)C(CC1=CC(=O)C=C1)NC(=O)c1cncn1-c1ccccn1]

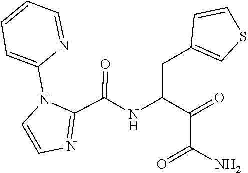 [SMILES: NC(=O)C(=O)C(Cc1ccsc1)NC(=O)c1nccn1-c1ccccn1]

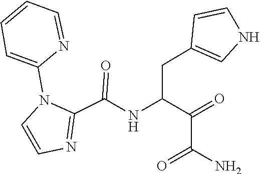 [SMILES: NC(=O)C(=O)C(Cc1cc[nH]c1)NC(=O)c1nccn1-c1ccccn1]

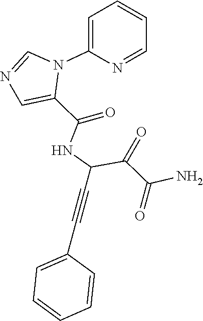 [SMILES: NC(=O)C(=O)C(C#Cc1ccccc1)NC(=O)c1cncn1-c1ccccn1]

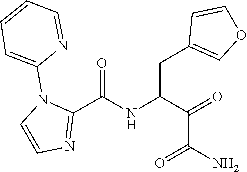 [SMILES: NC(=O)C(=O)C(Cc1ccoc1)NC(=O)c1nccn1-c1ccccn1]

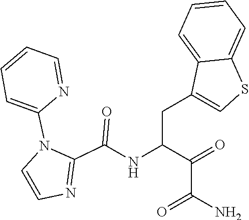 [SMILES: NC(=O)C(=O)C(Cc1csc2ccccc12)NC(=O)c1nccn1-c1ccccn1]

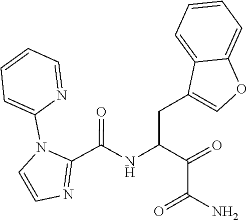 [SMILES: NC(=O)C(=O)C(Cc1coc2ccccc12)NC(=O)c1nccn1-c1ccccn1]

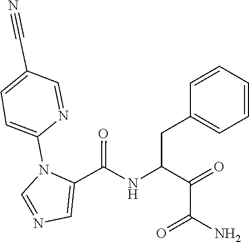 [SMILES: N#Cc1ccc(-n2cncc2C(=O)NC(Cc2ccccc2)C(=O)C(N)=O)nc1]

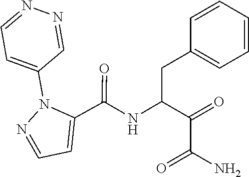 [SMILES: NC(=O)C(=O)C(Cc1ccccc1)NC(=O)c1ccnn1-c1ccnnc1]

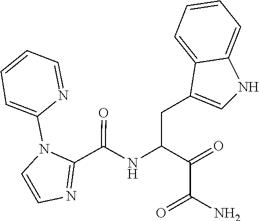 [SMILES: NC(=O)C(=O)C(Cc1c[nH]c2ccccc12)NC(=O)c1nccn1-c1ccccn1]

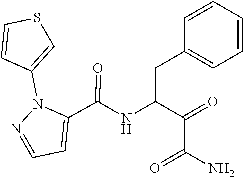 [SMILES: NC(=O)C(=O)C(Cc1ccccc1)NC(=O)c1ccnn1-c1ccsc1]

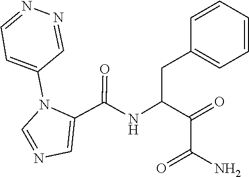 [SMILES: NC(=O)C(=O)C(Cc1ccccc1)NC(=O)c1cncn1-c1ccnnc1]

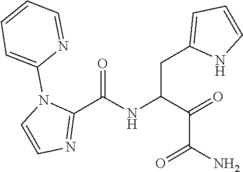 [SMILES: NC(=O)C(=O)C(Cc1ccc[nH]1)NC(=O)c1nccn1-c1ccccn1]

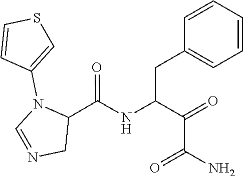 [SMILES: NC(=O)C(=O)C(Cc1ccccc1)NC(=O)C1CN=CN1c1ccsc1]

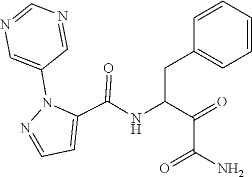 [SMILES: NC(=O)C(=O)C(Cc1ccccc1)NC(=O)c1ccnn1-c1cncnc1]

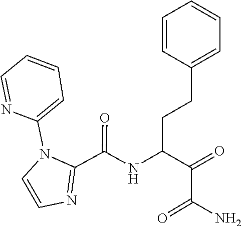 [SMILES: NC(=O)C(=O)C(CCc1ccccc1)NC(=O)c1nccn1-c1ccccn1]

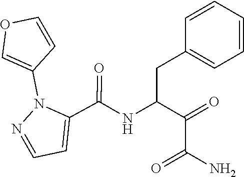 [SMILES: NC(=O)C(=O)C(Cc1ccccc1)NC(=O)c1ccnn1-c1ccoc1]

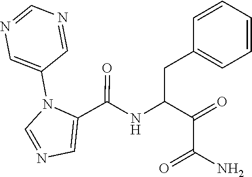 [SMILES: NC(=O)C(=O)C(Cc1ccccc1)NC(=O)c1cncn1-c1cncnc1]

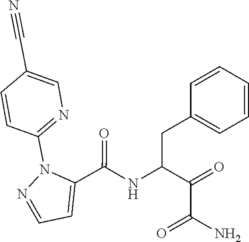 [SMILES: N#Cc1ccc(-n2nccc2C(=O)NC(Cc2ccccc2)C(=O)C(N)=O)nc1]

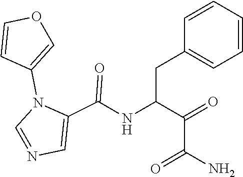 [SMILES: NC(=O)C(=O)C(Cc1ccccc1)NC(=O)c1cncn1-c1ccoc1]

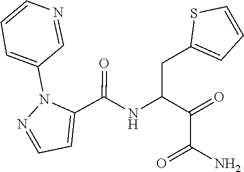 [SMILES: NC(=O)C(=O)C(Cc1cccs1)NC(=O)c1ccnn1-c1cccnc1]

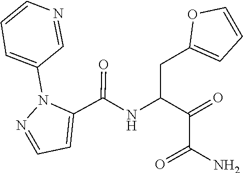 [SMILES: NC(=O)C(=O)C(Cc1ccco1)NC(=O)c1ccnn1-c1cccnc1]

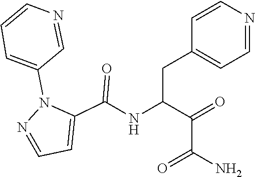 [SMILES: NC(=O)C(=O)C(Cc1ccncc1)NC(=O)c1ccnn1-c1cccnc1]

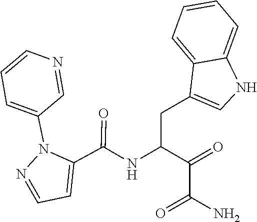 [SMILES: NC(=O)C(=O)C(Cc1c[nH]c2ccccc12)NC(=O)c1ccnn1-c1cccnc1]

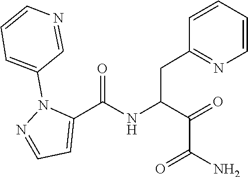 [SMILES: NC(=O)C(=O)C(Cc1ccccn1)NC(=O)c1ccnn1-c1cccnc1]

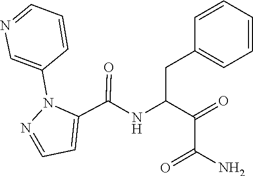 [SMILES: NC(=O)C(=O)C(Cc1ccccc1)NC(=O)c1ccnn1-c1cccnc1]

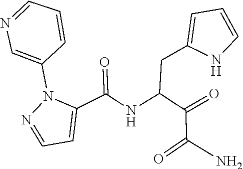 [SMILES: NC(=O)C(=O)C(Cc1ccc[nH]1)NC(=O)c1ccnn1-c1cccnc1]

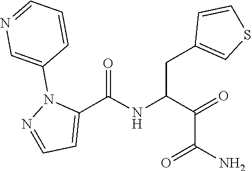 [SMILES: NC(=O)C(=O)C(Cc1ccsc1)NC(=O)c1ccnn1-c1cccnc1]

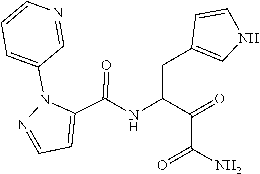 [SMILES: NC(=O)C(=O)C(Cc1cc[nH]c1)NC(=O)c1ccnn1-c1cccnc1]

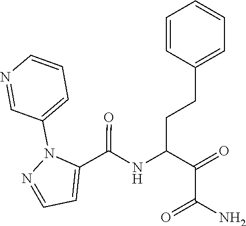 [SMILES: NC(=O)C(=O)C(CCc1ccccc1)NC(=O)c1ccnn1-c1cccnc1]

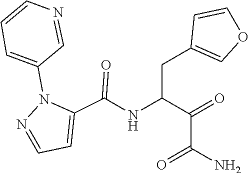 [SMILES: NC(=O)C(=O)C(Cc1ccoc1)NC(=O)c1ccnn1-c1cccnc1]

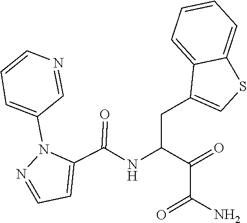 [SMILES: NC(=O)C(=O)C(Cc1csc2ccccc12)NC(=O)c1ccnn1-c1cccnc1]

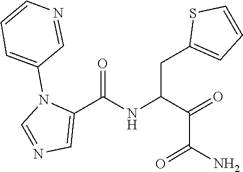 [SMILES: NC(=O)C(=O)C(Cc1cccs1)NC(=O)c1cncn1-c1cccnc1]

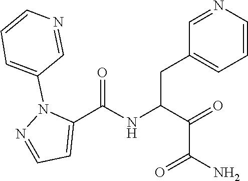 [SMILES: NC(=O)C(=O)C(Cc1cccnc1)NC(=O)c1ccnn1-c1cccnc1]

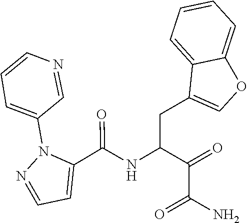 [SMILES: NC(=O)C(=O)C(Cc1coc2ccccc12)NC(=O)c1ccnn1-c1cccnc1]

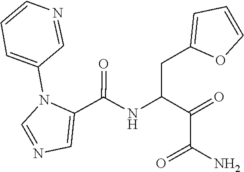 [SMILES: NC(=O)C(=O)C(Cc1ccco1)NC(=O)c1cncn1-c1cccnc1]

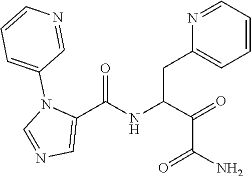 [SMILES: NC(=O)C(=O)C(Cc1ccccn1)NC(=O)c1cncn1-c1cccnc1]

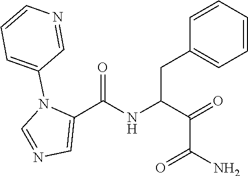 [SMILES: NC(=O)C(=O)C(Cc1ccccc1)NC(=O)c1cncn1-c1cccnc1]

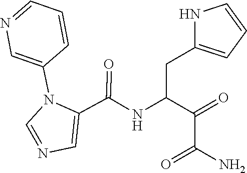 [SMILES: NC(=O)C(=O)C(Cc1ccc[nH]1)NC(=O)c1cncn1-c1cccnc1]

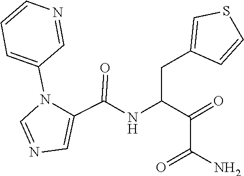 [SMILES: NC(=O)C(=O)C(Cc1ccsc1)NC(=O)c1cncn1-c1cccnc1]

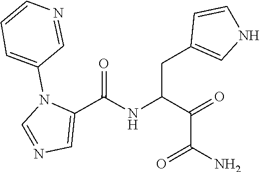 [SMILES: NC(=O)C(=O)C(Cc1cc[nH]c1)NC(=O)c1cncn1-c1cccnc1]

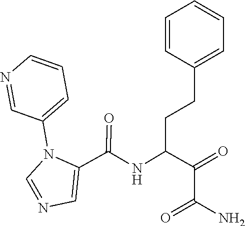 [SMILES: NC(=O)C(=O)C(CCc1ccccc1)NC(=O)c1cncn1-c1cccnc1]

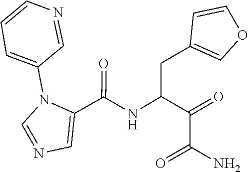 [SMILES: NC(=O)C(=O)C(Cc1ccoc1)NC(=O)c1cncn1-c1cccnc1]

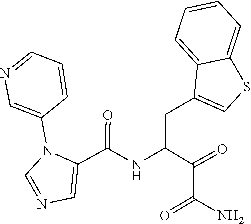 [SMILES: NC(=O)C(=O)C(Cc1csc2ccccc12)NC(=O)c1cncn1-c1cccnc1]

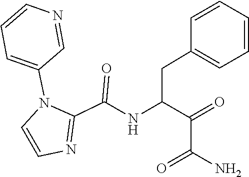 [SMILES: NC(=O)C(=O)C(Cc1ccccc1)NC(=O)c1nccn1-c1cccnc1]

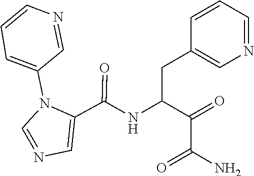 [SMILES: NC(=O)C(=O)C(Cc1cccnc1)NC(=O)c1cncn1-c1cccnc1]

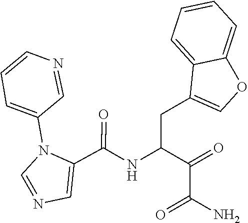 [SMILES: NC(=O)C(=O)C(Cc1coc2ccccc12)NC(=O)c1cncn1-c1cccnc1]

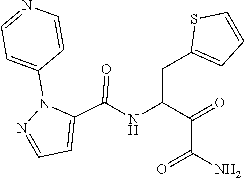 [SMILES: NC(=O)C(=O)C(Cc1cccs1)NC(=O)c1ccnn1-c1ccncc1]

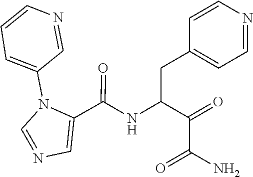 [SMILES: NC(=O)C(=O)C(Cc1ccncc1)NC(=O)c1cncn1-c1cccnc1]

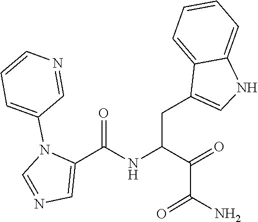 [SMILES: NC(=O)C(=O)C(Cc1c[nH]c2ccccc12)NC(=O)c1cncn1-c1cccnc1]

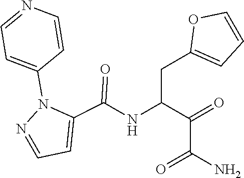 [SMILES: NC(=O)C(=O)C(Cc1ccco1)NC(=O)c1ccnn1-c1ccncc1]

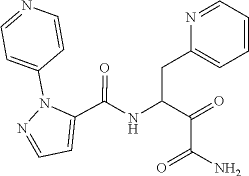 [SMILES: NC(=O)C(=O)C(Cc1ccccn1)NC(=O)c1ccnn1-c1ccncc1]

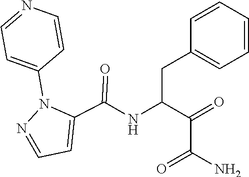 [SMILES: NC(=O)C(=O)C(Cc1ccccc1)NC(=O)c1ccnn1-c1ccncc1]

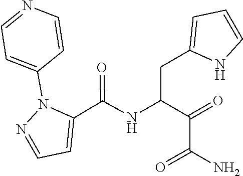 [SMILES: NC(=O)C(=O)C(Cc1ccc[nH]1)NC(=O)c1ccnn1-c1ccncc1]

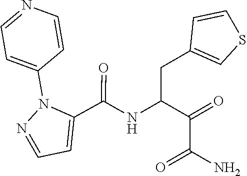 [SMILES: NC(=O)C(=O)C(Cc1ccsc1)NC(=O)c1ccnn1-c1ccncc1]

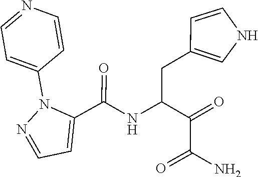 [SMILES: NC(=O)C(=O)C(Cc1cc[nH]c1)NC(=O)c1ccnn1-c1ccncc1]

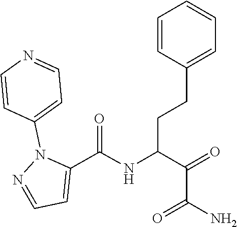 [SMILES: NC(=O)C(=O)C(CCc1ccccc1)NC(=O)c1ccnn1-c1ccncc1]

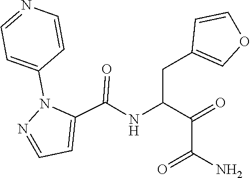 [SMILES: NC(=O)C(=O)C(Cc1ccoc1)NC(=O)c1ccnn1-c1ccncc1]

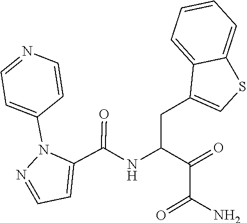 [SMILES: NC(=O)C(=O)C(Cc1csc2ccccc12)NC(=O)c1ccnn1-c1ccncc1]

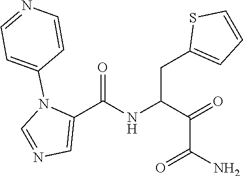 [SMILES: NC(=O)C(=O)C(Cc1cccs1)NC(=O)c1cncn1-c1ccncc1]

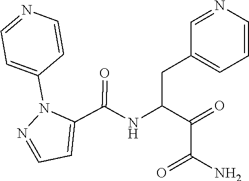 [SMILES: NC(=O)C(=O)C(Cc1cccnc1)NC(=O)c1ccnn1-c1ccncc1]

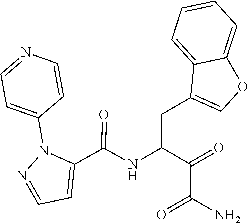 [SMILES: NC(=O)C(=O)C(Cc1coc2ccccc12)NC(=O)c1ccnn1-c1ccncc1]

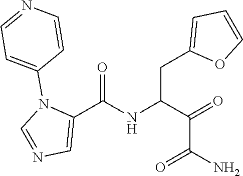 [SMILES: NC(=O)C(=O)C(Cc1ccco1)NC(=O)c1cncn1-c1ccncc1]

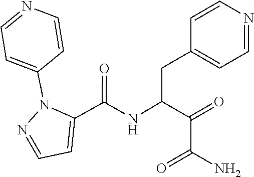 [SMILES: NC(=O)C(=O)C(Cc1ccncc1)NC(=O)c1ccnn1-c1ccncc1]

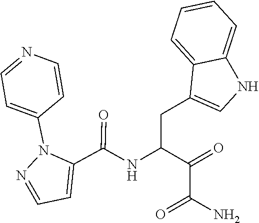 [SMILES: NC(=O)C(=O)C(Cc1c[nH]c2ccccc12)NC(=O)c1ccnn1-c1ccncc1]

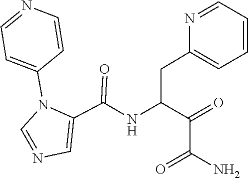 [SMILES: NC(=O)C(=O)C(Cc1ccccn1)NC(=O)c1cncn1-c1ccncc1]

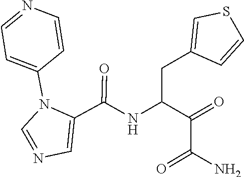 [SMILES: NC(=O)C(=O)C(Cc1ccsc1)NC(=O)c1cncn1-c1ccncc1]

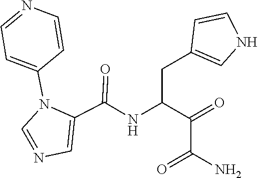 [SMILES: NC(=O)C(=O)C(Cc1cc[nH]c1)NC(=O)c1cncn1-c1ccncc1]

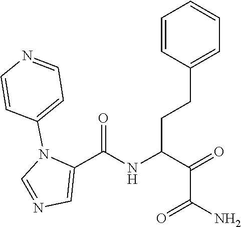 [SMILES: NC(=O)C(=O)C(CCc1ccccc1)NC(=O)c1cncn1-c1ccncc1]

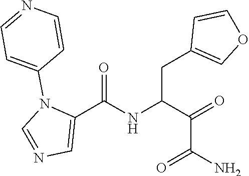 [SMILES: NC(=O)C(=O)C(Cc1ccoc1)NC(=O)c1cncn1-c1ccncc1]

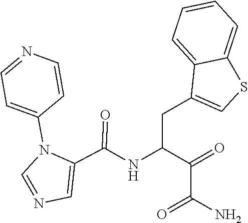 [SMILES: NC(=O)C(=O)C(Cc1csc2ccccc12)NC(=O)c1cncn1-c1ccncc1]

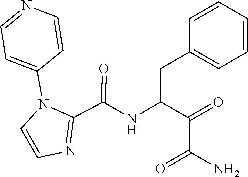 [SMILES: NC(=O)C(=O)C(Cc1ccccc1)NC(=O)c1nccn1-c1ccncc1]

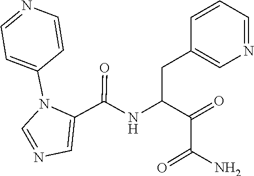 [SMILES: NC(=O)C(=O)C(Cc1cccnc1)NC(=O)c1cncn1-c1ccncc1]

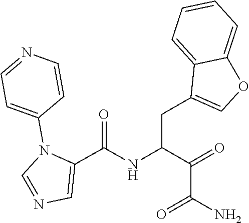 [SMILES: NC(=O)C(=O)C(Cc1coc2ccccc12)NC(=O)c1cncn1-c1ccncc1]

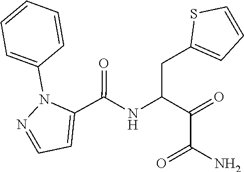 [SMILES: NC(=O)C(=O)C(Cc1cccs1)NC(=O)c1ccnn1-c1ccccc1]

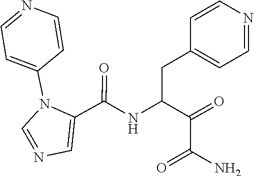 [SMILES: NC(=O)C(=O)C(Cc1ccncc1)NC(=O)c1cncn1-c1ccncc1]

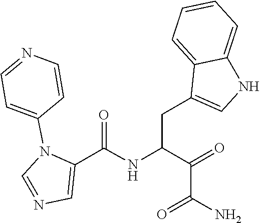 [SMILES: NC(=O)C(=O)C(Cc1c[nH]c2ccccc12)NC(=O)c1cncn1-c1ccncc1]

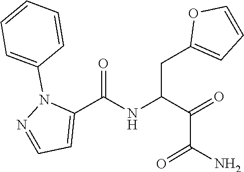 [SMILES: NC(=O)C(=O)C(Cc1ccco1)NC(=O)c1ccnn1-c1ccccc1]

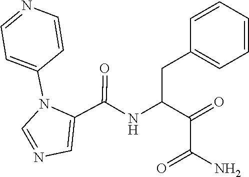 [SMILES: NC(=O)C(=O)C(Cc1ccccc1)NC(=O)c1cncn1-c1ccncc1]

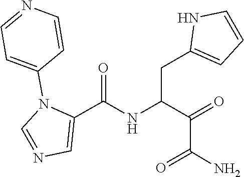 [SMILES: NC(=O)C(=O)C(Cc1ccc[nH]1)NC(=O)c1cncn1-c1ccncc1]

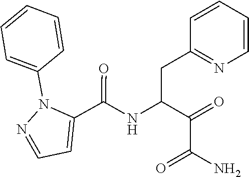 [SMILES: NC(=O)C(=O)C(Cc1ccccn1)NC(=O)c1ccnn1-c1ccccc1]

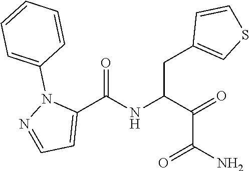 [SMILES: NC(=O)C(=O)C(Cc1ccsc1)NC(=O)c1ccnn1-c1ccccc1]

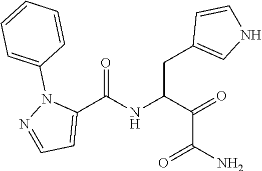 [SMILES: NC(=O)C(=O)C(Cc1cc[nH]c1)NC(=O)c1ccnn1-c1ccccc1]

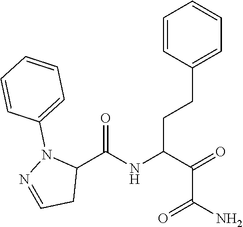 [SMILES: NC(=O)C(=O)C(CCc1ccccc1)NC(=O)C1CC=NN1c1ccccc1]

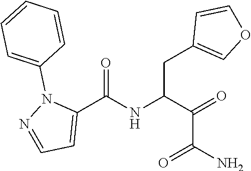 [SMILES: NC(=O)C(=O)C(Cc1ccoc1)NC(=O)c1ccnn1-c1ccccc1]

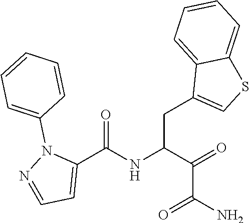 [SMILES: NC(=O)C(=O)C(Cc1csc2ccccc12)NC(=O)c1ccnn1-c1ccccc1]

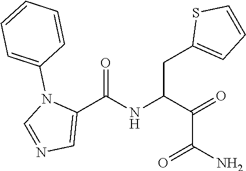 [SMILES: NC(=O)C(=O)C(Cc1cccs1)NC(=O)c1cncn1-c1ccccc1]

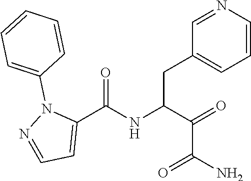 [SMILES: NC(=O)C(=O)C(Cc1cccnc1)NC(=O)c1ccnn1-c1ccccc1]

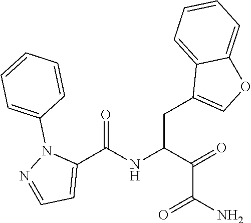 [SMILES: NC(=O)C(=O)C(Cc1coc2ccccc12)NC(=O)c1ccnn1-c1ccccc1]

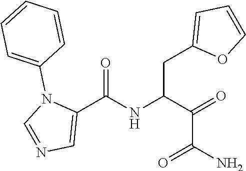 [SMILES: NC(=O)C(=O)C(Cc1ccco1)NC(=O)c1cncn1-c1ccccc1]

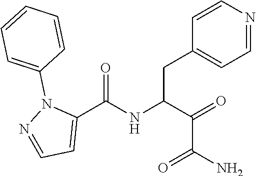 [SMILES: NC(=O)C(=O)C(Cc1ccncc1)NC(=O)c1ccnn1-c1ccccc1]

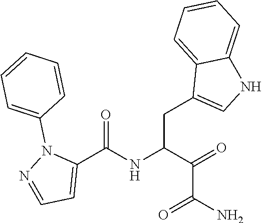 [SMILES: NC(=O)C(=O)C(Cc1c[nH]c2ccccc12)NC(=O)c1ccnn1-c1ccccc1]

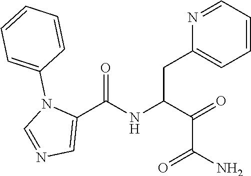 [SMILES: NC(=O)C(=O)C(Cc1ccccn1)NC(=O)c1cncn1-c1ccccc1]

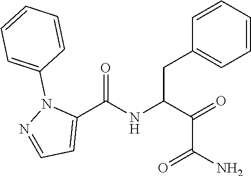 [SMILES: NC(=O)C(=O)C(Cc1ccccc1)NC(=O)c1ccnn1-c1ccccc1]

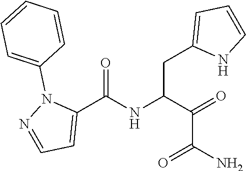 [SMILES: NC(=O)C(=O)C(Cc1ccc[nH]1)NC(=O)c1ccnn1-c1ccccc1]

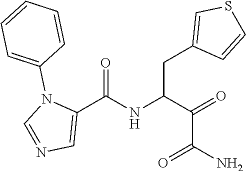 [SMILES: NC(=O)C(=O)C(Cc1ccsc1)NC(=O)c1cncn1-c1ccccc1]

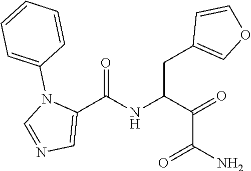 [SMILES: NC(=O)C(=O)C(Cc1ccoc1)NC(=O)c1cncn1-c1ccccc1]

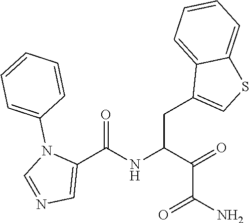 [SMILES: NC(=O)C(=O)C(Cc1csc2ccccc12)NC(=O)c1cncn1-c1ccccc1]

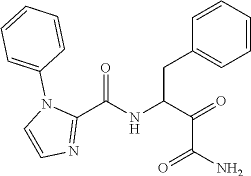 [SMILES: NC(=O)C(=O)C(Cc1ccccc1)NC(=O)c1nccn1-c1ccccc1]

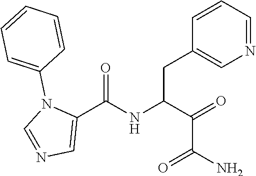 [SMILES: NC(=O)C(=O)C(Cc1cccnc1)NC(=O)c1cncn1-c1ccccc1]

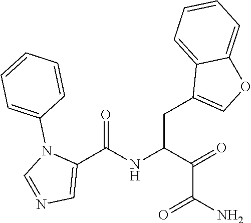 [SMILES: NC(=O)C(=O)C(Cc1coc2ccccc12)NC(=O)c1cncn1-c1ccccc1]

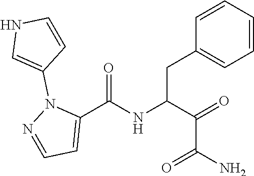 [SMILES: NC(=O)C(=O)C(Cc1ccccc1)NC(=O)c1ccnn1-c1cc[nH]c1]

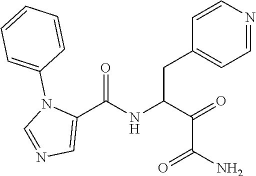 [SMILES: NC(=O)C(=O)C(Cc1ccncc1)NC(=O)c1cncn1-c1ccccc1]

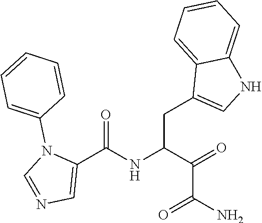 [SMILES: NC(=O)C(=O)C(Cc1c[nH]c2ccccc12)NC(=O)c1cncn1-c1ccccc1]

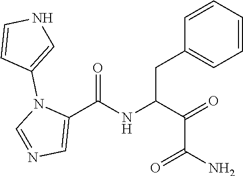 [SMILES: NC(=O)C(=O)C(Cc1ccccc1)NC(=O)c1cncn1-c1cc[nH]c1]

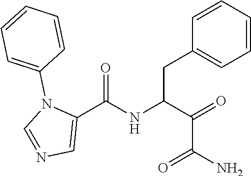 [SMILES: NC(=O)C(=O)C(Cc1ccccc1)NC(=O)c1cncn1-c1ccccc1]

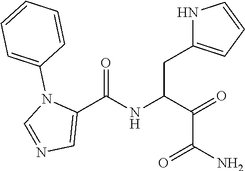 [SMILES: NC(=O)C(=O)C(Cc1ccc[nH]1)NC(=O)c1cncn1-c1ccccc1]

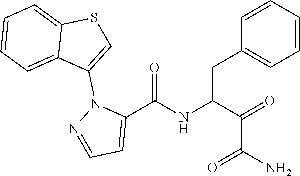 [SMILES: NC(=O)C(=O)C(Cc1ccccc1)NC(=O)c1ccnn1-c1csc2ccccc12]

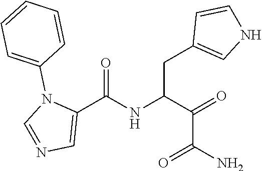 [SMILES: NC(=O)C(=O)C(Cc1cc[nH]c1)NC(=O)c1cncn1-c1ccccc1]

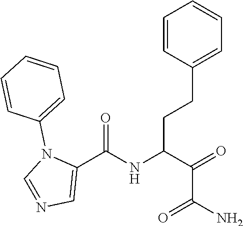 [SMILES: NC(=O)C(=O)C(CCc1ccccc1)NC(=O)c1cncn1-c1ccccc1]

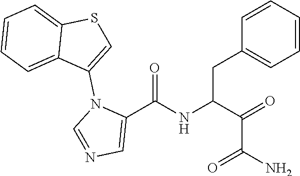 [SMILES: NC(=O)C(=O)C(Cc1ccccc1)NC(=O)c1cncn1-c1csc2ccccc12]

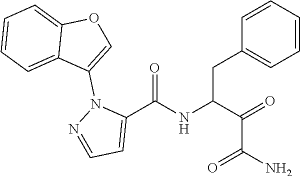 [SMILES: NC(=O)C(=O)C(Cc1ccccc1)NC(=O)c1ccnn1-c1coc2ccccc12]

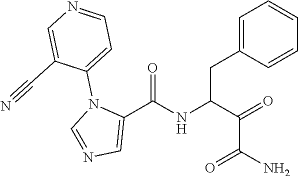 [SMILES: N#Cc1cnccc1-n1cncc1C(=O)NC(Cc1ccccc1)C(=O)C(N)=O]

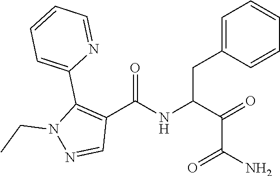 [SMILES: CCn1ncc(C(=O)NC(Cc2ccccc2)C(=O)C(N)=O)c1-c1ccccn1]

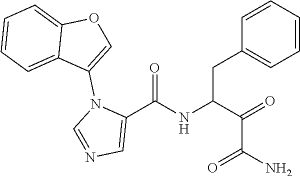 [SMILES: NC(=O)C(=O)C(Cc1ccccc1)NC(=O)c1cncn1-c1coc2ccccc12]

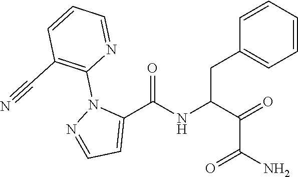 [SMILES: N#Cc1cccnc1-n1nccc1C(=O)NC(Cc1ccccc1)C(=O)C(N)=O]

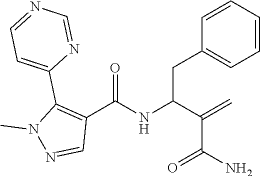 [SMILES: C=C(C(N)=O)C(Cc1ccccc1)NC(=O)c1cnn(C)c1-c1ccncn1]

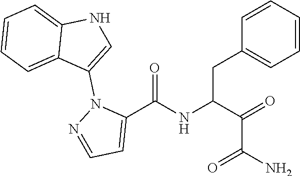 [SMILES: NC(=O)C(=O)C(Cc1ccccc1)NC(=O)c1ccnn1-c1c[nH]c2ccccc12]

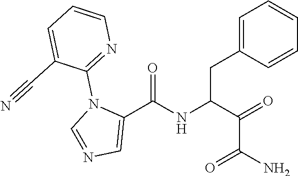 [SMILES: N#Cc1cccnc1-n1cncc1C(=O)NC(Cc1ccccc1)C(=O)C(N)=O]

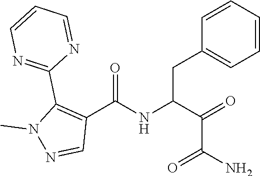 [SMILES: Cn1ncc(C(=O)NC(Cc2ccccc2)C(=O)C(N)=O)c1-c1ncccn1]

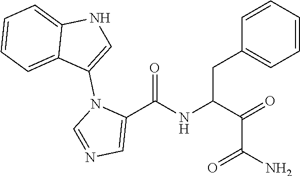 [SMILES: NC(=O)C(=O)C(Cc1ccccc1)NC(=O)c1cncn1-c1c[nH]c2ccccc12]

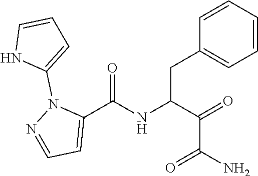 [SMILES: NC(=O)C(=O)C(Cc1ccccc1)NC(=O)c1ccnn1-c1ccc[nH]1]

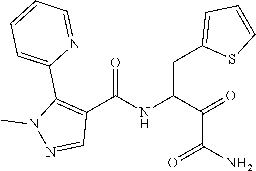 [SMILES: Cn1ncc(C(=O)NC(Cc2cccs2)C(=O)C(N)=O)c1-c1ccccn1]

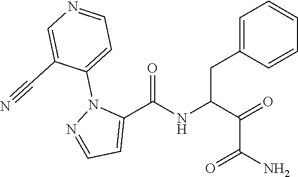 [SMILES: N#Cc1cnccc1-n1nccc1C(=O)NC(Cc1ccccc1)C(=O)C(N)=O]

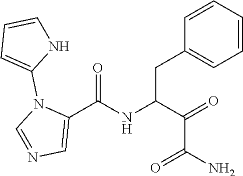 [SMILES: NC(=O)C(=O)C(Cc1ccccc1)NC(=O)c1cncn1-c1ccc[nH]1]

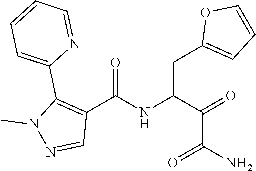 [SMILES: Cn1ncc(C(=O)NC(Cc2ccco2)C(=O)C(N)=O)c1-c1ccccn1]

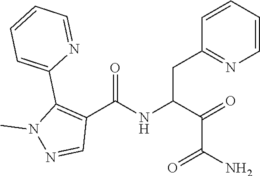 [SMILES: Cn1ncc(C(=O)NC(Cc2ccccn2)C(=O)C(N)=O)c1-c1ccccn1]

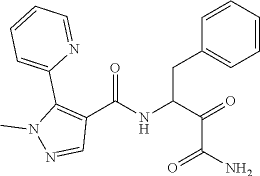 [SMILES: Cn1ncc(C(=O)NC(Cc2ccccc2)C(=O)C(N)=O)c1-c1ccccn1]

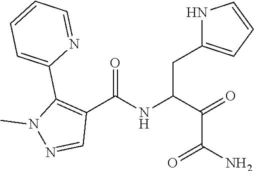 [SMILES: Cn1ncc(C(=O)NC(Cc2ccc[nH]2)C(=O)C(N)=O)c1-c1ccccn1]

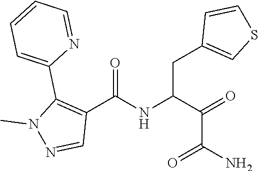 [SMILES: Cn1ncc(C(=O)NC(Cc2ccsc2)C(=O)C(N)=O)c1-c1ccccn1]

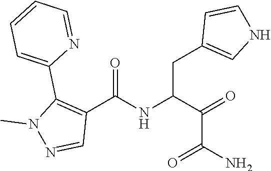 [SMILES: Cn1ncc(C(=O)NC(Cc2cc[nH]c2)C(=O)C(N)=O)c1-c1ccccn1]

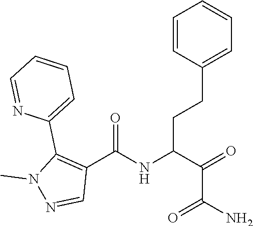 [SMILES: Cn1ncc(C(=O)NC(CCc2ccccc2)C(=O)C(N)=O)c1-c1ccccn1]

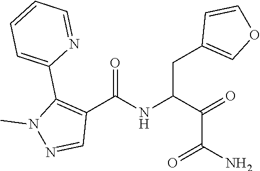 [SMILES: Cn1ncc(C(=O)NC(Cc2ccoc2)C(=O)C(N)=O)c1-c1ccccn1]

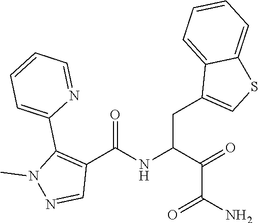 [SMILES: Cn1ncc(C(=O)NC(Cc2csc3ccccc23)C(=O)C(N)=O)c1-c1ccccn1]

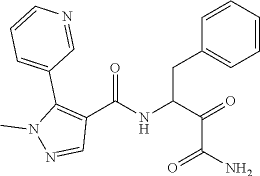 [SMILES: Cn1ncc(C(=O)NC(Cc2ccccc2)C(=O)C(N)=O)c1-c1cccnc1]

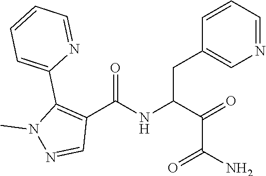 [SMILES: Cn1ncc(C(=O)NC(Cc2cccnc2)C(=O)C(N)=O)c1-c1ccccn1]

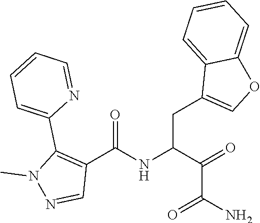 [SMILES: Cn1ncc(C(=O)NC(Cc2coc3ccccc23)C(=O)C(N)=O)c1-c1ccccn1]

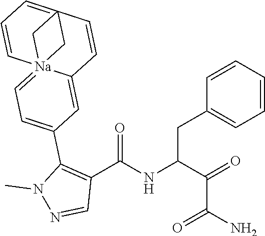

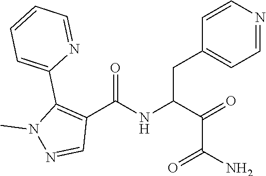 [SMILES: Cn1ncc(C(=O)NC(Cc2ccncc2)C(=O)C(N)=O)c1-c1ccccn1]

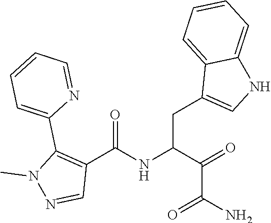 [SMILES: Cn1ncc(C(=O)NC(Cc2c[nH]c3ccccc23)C(=O)C(N)=O)c1-c1ccccn1]

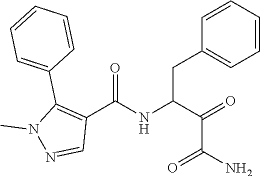 [SMILES: Cn1ncc(C(=O)NC(Cc2ccccc2)C(=O)C(N)=O)c1-c1ccccc1]

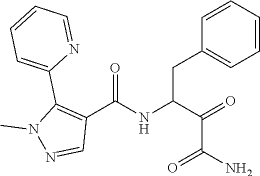 [SMILES: Cn1ncc(C(=O)NC(Cc2ccccc2)C(=O)C(N)=O)c1-c1ccccn1]

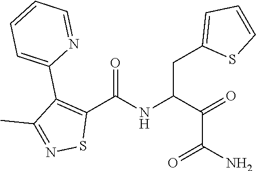 [SMILES: Cc1nsc(C(=O)NC(Cc2cccs2)C(=O)C(N)=O)c1-c1ccccn1]

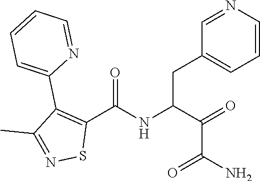 [SMILES: Cc1nsc(C(=O)NC(Cc2cccnc2)C(=O)C(N)=O)c1-c1ccccn1]

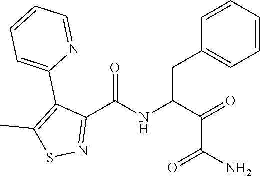 [SMILES: Cc1snc(C(=O)NC(Cc2ccccc2)C(=O)C(N)=O)c1-c1ccccn1]

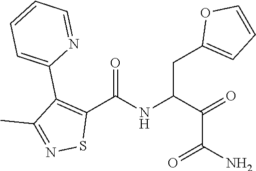 [SMILES: Cc1nsc(C(=O)NC(Cc2ccco2)C(=O)C(N)=O)c1-c1ccccn1]

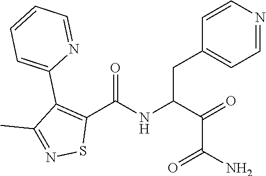 [SMILES: Cc1nsc(C(=O)NC(Cc2ccncc2)C(=O)C(N)=O)c1-c1ccccn1]

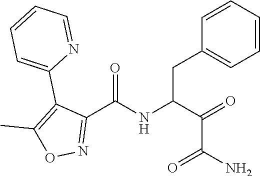 [SMILES: Cc1onc(C(=O)NC(Cc2ccccc2)C(=O)C(N)=O)c1-c1ccccn1]

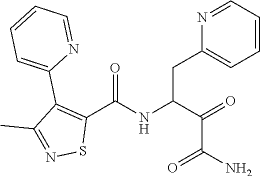 [SMILES: Cc1nsc(C(=O)NC(Cc2ccccn2)C(=O)C(N)=O)c1-c1ccccn1]

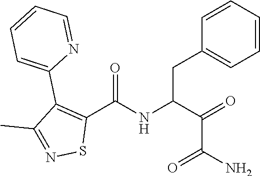 [SMILES: Cc1nsc(C(=O)NC(Cc2ccccc2)C(=O)C(N)=O)c1-c1ccccn1]

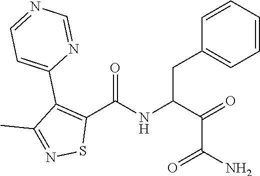 [SMILES: Cc1nsc(C(=O)NC(Cc2ccccc2)C(=O)C(N)=O)c1-c1ccncn1]

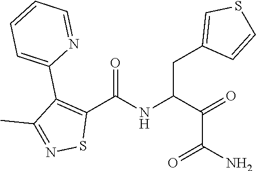 [SMILES: Cc1nsc(C(=O)NC(Cc2ccsc2)C(=O)C(N)=O)c1-c1ccccn1]

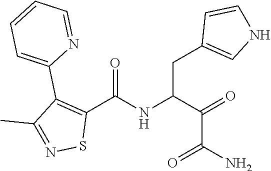 [SMILES: Cc1nsc(C(=O)NC(Cc2cc[nH]c2)C(=O)C(N)=O)c1-c1ccccn1]

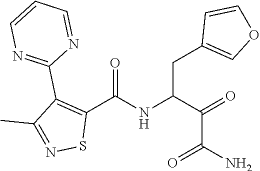 [SMILES: Cc1nsc(C(=O)NC(Cc2ccoc2)C(=O)C(N)=O)c1-c1ncccn1]

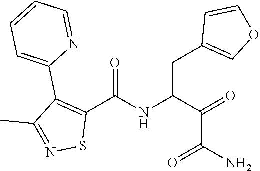 [SMILES: Cc1nsc(C(=O)NC(Cc2ccoc2)C(=O)C(N)=O)c1-c1ccccn1]

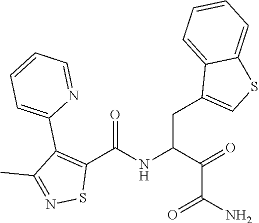 [SMILES: Cc1nsc(C(=O)NC(Cc2csc3ccccc23)C(=O)C(N)=O)c1-c1ccccn1]

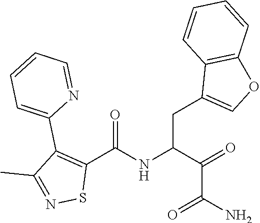 [SMILES: Cc1nsc(C(=O)NC(Cc2coc3ccccc23)C(=O)C(N)=O)c1-c1ccccn1]

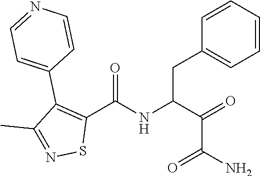 [SMILES: Cc1nsc(C(=O)NC(Cc2ccccc2)C(=O)C(N)=O)c1-c1ccncc1]

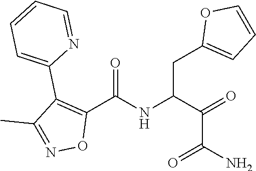 [SMILES: Cc1noc(C(=O)NC(Cc2ccco2)C(=O)C(N)=O)c1-c1ccccn1]

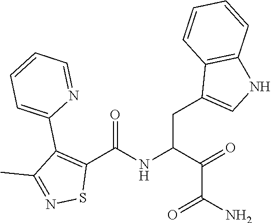 [SMILES: Cc1nsc(C(=O)NC(Cc2c[nH]c3ccccc23)C(=O)C(N)=O)c1-c1ccccn1]

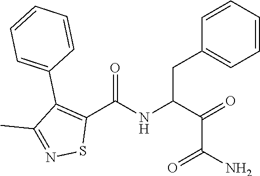 [SMILES: Cc1nsc(C(=O)NC(Cc2ccccc2)C(=O)C(N)=O)c1-c1ccccc1]

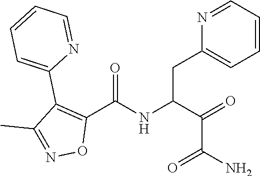 [SMILES: Cc1noc(C(=O)NC(Cc2ccccn2)C(=O)C(N)=O)c1-c1ccccn1]

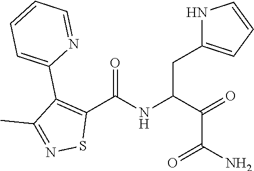 [SMILES: Cc1nsc(C(=O)NC(Cc2ccc[nH]2)C(=O)C(N)=O)c1-c1ccccn1]

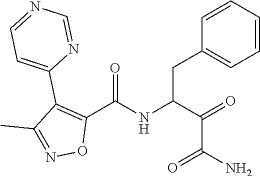 [SMILES: Cc1noc(C(=O)NC(Cc2ccccc2)C(=O)C(N)=O)c1-c1ccncn1]

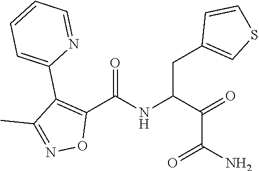 [SMILES: Cc1noc(C(=O)NC(Cc2ccsc2)C(=O)C(N)=O)c1-c1ccccn1]

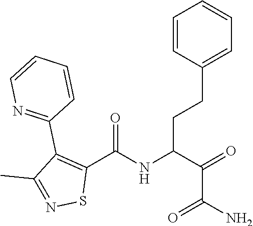 [SMILES: Cc1nsc(C(=O)NC(CCc2ccccc2)C(=O)C(N)=O)c1-c1ccccn1]

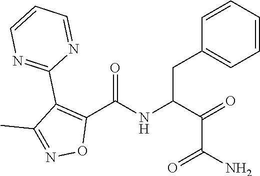 [SMILES: Cc1noc(C(=O)NC(Cc2ccccc2)C(=O)C(N)=O)c1-c1ncccn1]

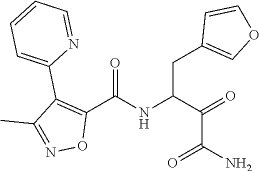 [SMILES: Cc1noc(C(=O)NC(Cc2ccoc2)C(=O)C(N)=O)c1-c1ccccn1]

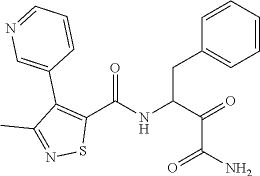 [SMILES: Cc1nsc(C(=O)NC(Cc2ccccc2)C(=O)C(N)=O)c1-c1cccnc1]

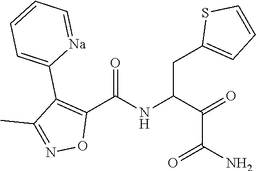

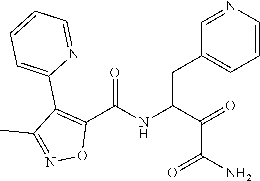 [SMILES: Cc1noc(C(=O)NC(Cc2cccnc2)C(=O)C(N)=O)c1-c1ccccn1]

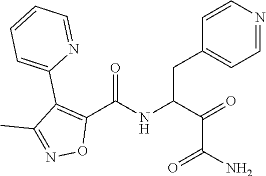 [SMILES: Cc1noc(C(=O)NC(Cc2ccncc2)C(=O)C(N)=O)c1-c1ccccn1]

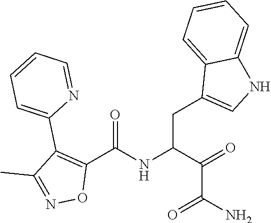 [SMILES: Cc1noc(C(=O)NC(Cc2c[nH]c3ccccc23)C(=O)C(N)=O)c1-c1ccccn1]

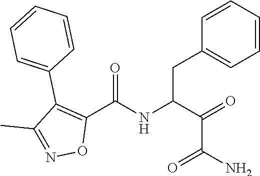 [SMILES: Cc1noc(C(=O)NC(Cc2ccccc2)C(=O)C(N)=O)c1-c1ccccc1]

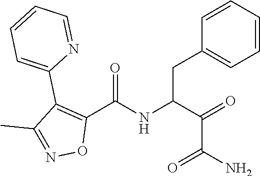 [SMILES: Cc1noc(C(=O)NC(Cc2ccccc2)C(=O)C(N)=O)c1-c1ccccn1]

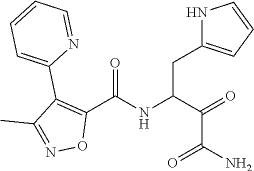 [SMILES: Cc1noc(C(=O)NC(Cc2ccc[nH]2)C(=O)C(N)=O)c1-c1ccccn1]

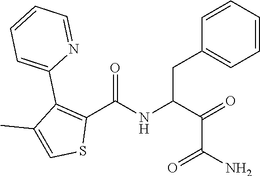 [SMILES: Cc1csc(C(=O)NC(Cc2ccccc2)C(=O)C(N)=O)c1-c1ccccn1]

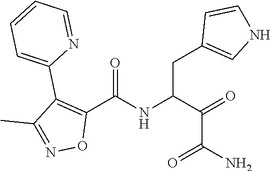 [SMILES: Cc1noc(C(=O)NC(Cc2cc[nH]c2)C(=O)C(N)=O)c1-c1ccccn1]

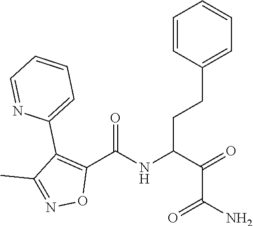 [SMILES: Cc1noc(C(=O)NC(CCc2ccccc2)C(=O)C(N)=O)c1-c1ccccn1]

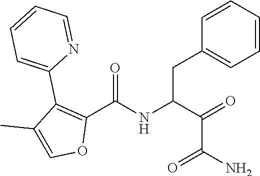 [SMILES: Cc1coc(C(=O)NC(Cc2ccccc2)C(=O)C(N)=O)c1-c1ccccn1]

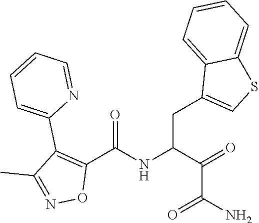 [SMILES: Cc1noc(C(=O)NC(Cc2csc3ccccc23)C(=O)C(N)=O)c1-c1ccccn1]

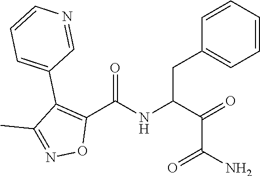 [SMILES: Cc1noc(C(=O)NC(Cc2ccccc2)C(=O)C(N)=O)c1-c1cccnc1]

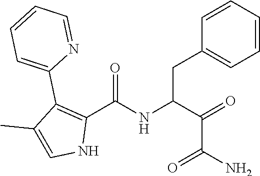 [SMILES: Cc1c[nH]c(C(=O)NC(Cc2ccccc2)C(=O)C(N)=O)c1-c1ccccn1]

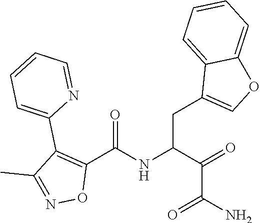 [SMILES: Cc1noc(C(=O)NC(Cc2coc3ccccc23)C(=O)C(N)=O)c1-c1ccccn1]

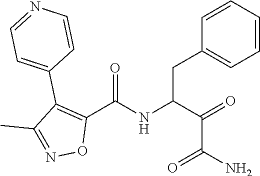 [SMILES: Cc1noc(C(=O)NC(Cc2ccccc2)C(=O)C(N)=O)c1-c1ccncc1]

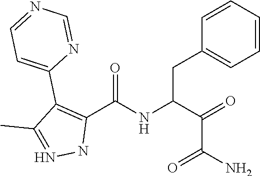 [SMILES: Cc1n[nH]c(C(=O)NC(Cc2ccccc2)C(=O)C(N)=O)c1-c1ccncn1]

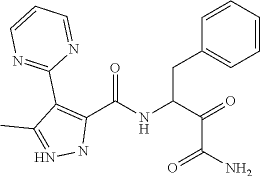 [SMILES: Cc1n[nH]c(C(=O)NC(Cc2ccccc2)C(=O)C(N)=O)c1-c1ncccn1]

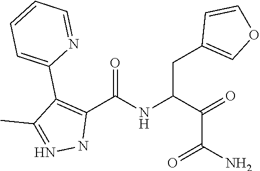 [SMILES: Cc1n[nH]c(C(=O)NC(Cc2ccoc2)C(=O)C(N)=O)c1-c1ccccn1]

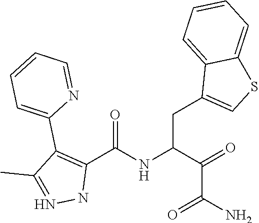 [SMILES: Cc1n[nH]c(C(=O)NC(Cc2csc3ccccc23)C(=O)C(N)=O)c1-c1ccccn1]

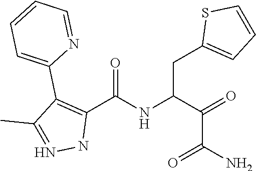 [SMILES: Cc1n[nH]c(C(=O)NC(Cc2cccs2)C(=O)C(N)=O)c1-c1ccccn1]

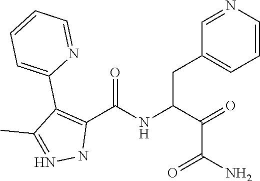 [SMILES: Cc1n[nH]c(C(=O)NC(Cc2cccnc2)C(=O)C(N)=O)c1-c1ccccn1]

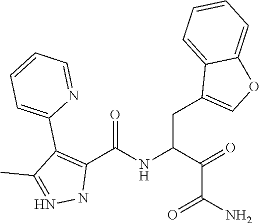 [SMILES: Cc1n[nH]c(C(=O)NC(Cc2coc3ccccc23)C(=O)C(N)=O)c1-c1ccccn1]

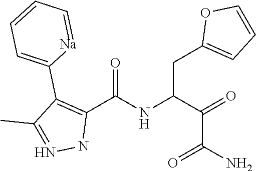

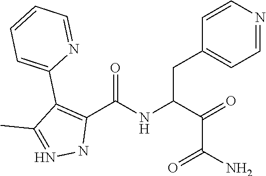 [SMILES: Cc1n[nH]c(C(=O)NC(Cc2ccncc2)C(=O)C(N)=O)c1-c1ccccn1]

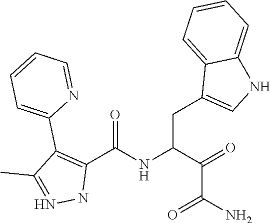 [SMILES: Cc1n[nH]c(C(=O)NC(Cc2c[nH]c3ccccc23)C(=O)C(N)=O)c1-c1ccccn1]

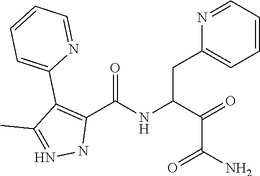 [SMILES: Cc1n[nH]c(C(=O)NC(Cc2ccccn2)C(=O)C(N)=O)c1-c1ccccn1]

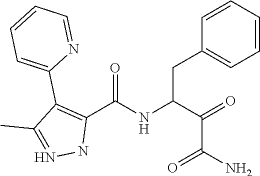 [SMILES: Cc1n[nH]c(C(=O)NC(Cc2ccccc2)C(=O)C(N)=O)c1-c1ccccn1]

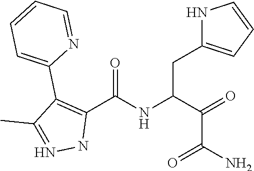 [SMILES: Cc1n[nH]c(C(=O)NC(Cc2ccc[nH]2)C(=O)C(N)=O)c1-c1ccccn1]

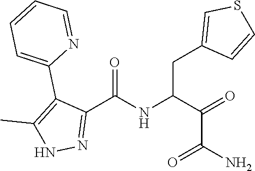 [SMILES: Cc1[nH]nc(C(=O)NC(Cc2ccsc2)C(=O)C(N)=O)c1-c1ccccn1]

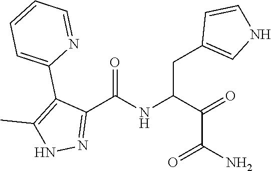 [SMILES: Cc1[nH]nc(C(=O)NC(Cc2cc[nH]c2)C(=O)C(N)=O)c1-c1ccccn1]

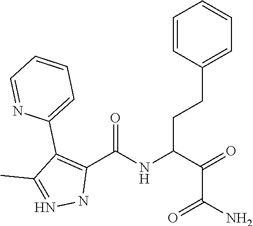 [SMILES: Cc1n[nH]c(C(=O)NC(CCc2ccccc2)C(=O)C(N)=O)c1-c1ccccn1]

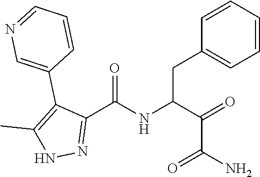 [SMILES: Cc1[nH]nc(C(=O)NC(Cc2ccccc2)C(=O)C(N)=O)c1-c1cccnc1]

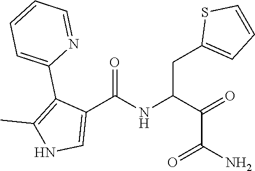 [SMILES: Cc1[nH]cc(C(=O)NC(Cc2cccs2)C(=O)C(N)=O)c1-c1ccccn1]

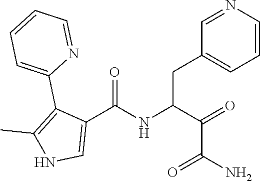 [SMILES: Cc1[nH]cc(C(=O)NC(Cc2cccnc2)C(=O)C(N)=O)c1-c1ccccn1]

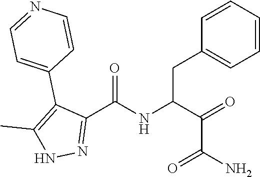 [SMILES: Cc1[nH]nc(C(=O)NC(Cc2ccccc2)C(=O)C(N)=O)c1-c1ccncc1]

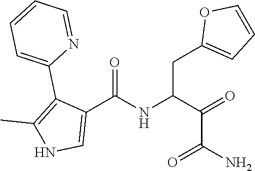 [SMILES: Cc1[nH]cc(C(=O)NC(Cc2ccco2)C(=O)C(N)=O)c1-c1ccccn1]

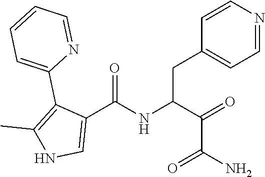 [SMILES: Cc1[nH]cc(C(=O)NC(Cc2ccncc2)C(=O)C(N)=O)c1-c1ccccn1]

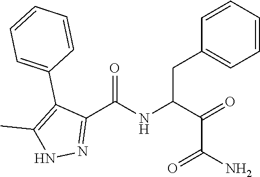 [SMILES: Cc1[nH]nc(C(=O)NC(Cc2ccccc2)C(=O)C(N)=O)c1-c1ccccc1]

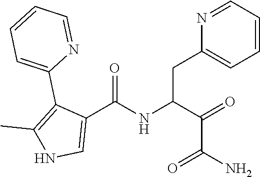 [SMILES: Cc1[nH]cc(C(=O)NC(Cc2ccccn2)C(=O)C(N)=O)c1-c1ccccn1]

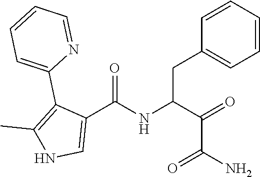 [SMILES: Cc1[nH]cc(C(=O)NC(Cc2ccccc2)C(=O)C(N)=O)c1-c1ccccn1]

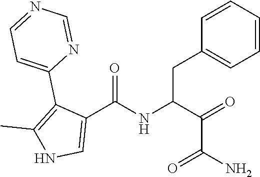 [SMILES: Cc1[nH]cc(C(=O)NC(Cc2ccccc2)C(=O)C(N)=O)c1-c1ccncn1]

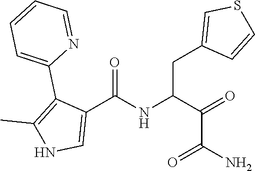 [SMILES: Cc1[nH]cc(C(=O)NC(Cc2ccsc2)C(=O)C(N)=O)c1-c1ccccn1]

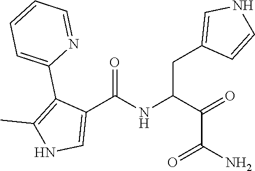 [SMILES: Cc1[nH]cc(C(=O)NC(Cc2cc[nH]c2)C(=O)C(N)=O)c1-c1ccccn1]

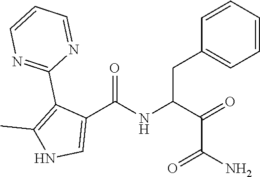 [SMILES: Cc1[nH]cc(C(=O)NC(Cc2ccccc2)C(=O)C(N)=O)c1-c1ncccn1]

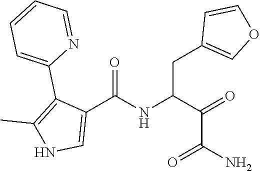 [SMILES: Cc1[nH]cc(C(=O)NC(Cc2ccoc2)C(=O)C(N)=O)c1-c1ccccn1]

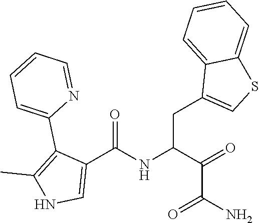 [SMILES: Cc1[nH]cc(C(=O)NC(Cc2csc3ccccc23)C(=O)C(N)=O)c1-c1ccccn1]

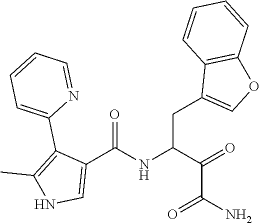 [SMILES: Cc1[nH]cc(C(=O)NC(Cc2coc3ccccc23)C(=O)C(N)=O)c1-c1ccccn1]

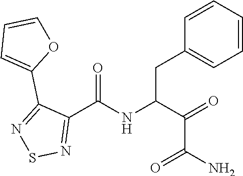 [SMILES: NC(=O)C(=O)C(Cc1ccccc1)NC(=O)c1nsnc1-c1ccco1]

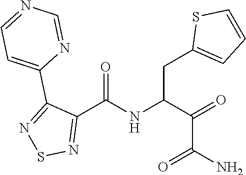 [SMILES: NC(=O)C(=O)C(Cc1cccs1)NC(=O)c1nsnc1-c1ccncn1]

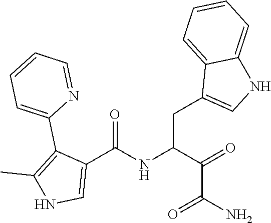 [SMILES: Cc1[nH]cc(C(=O)NC(Cc2c[nH]c3ccccc23)C(=O)C(N)=O)c1-c1ccccn1]

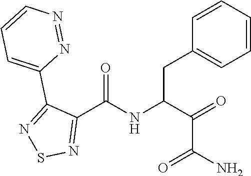 [SMILES: NC(=O)C(=O)C(Cc1ccccc1)NC(=O)c1nsnc1-c1cccnn1]

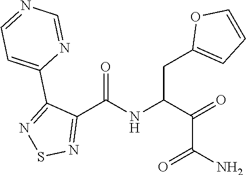 [SMILES: NC(=O)C(=O)C(Cc1ccco1)NC(=O)c1nsnc1-c1ccncn1]

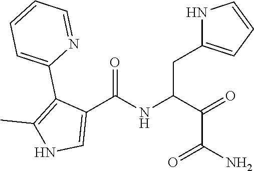 [SMILES: Cc1[nH]cc(C(=O)NC(Cc2ccc[nH]2)C(=O)C(N)=O)c1-c1ccccn1]

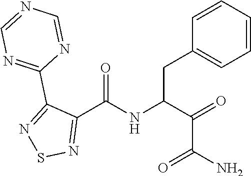 [SMILES: NC(=O)C(=O)C(Cc1ccccc1)NC(=O)c1nsnc1-c1ncncn1]

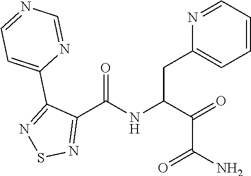 [SMILES: NC(=O)C(=O)C(Cc1ccccn1)NC(=O)c1nsnc1-c1ccncn1]

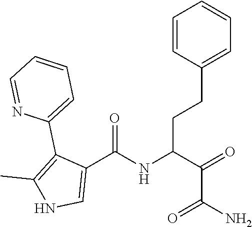 [SMILES: Cc1[nH]cc(C(=O)NC(CCc2ccccc2)C(=O)C(N)=O)c1-c1ccccn1]

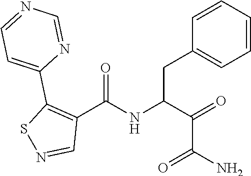 [SMILES: NC(=O)C(=O)C(Cc1ccccc1)NC(=O)c1cnsc1-c1ccncn1]

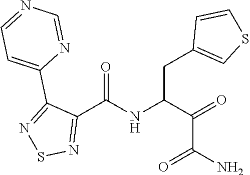 [SMILES: NC(=O)C(=O)C(Cc1ccsc1)NC(=O)c1nsnc1-c1ccncn1]

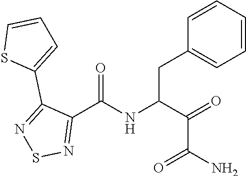 [SMILES: NC(=O)C(=O)C(Cc1ccccc1)NC(=O)c1nsnc1-c1cccs1]

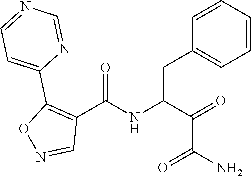 [SMILES: NC(=O)C(=O)C(Cc1ccccc1)NC(=O)c1cnoc1-c1ccncn1]

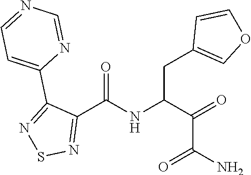 [SMILES: NC(=O)C(=O)C(Cc1ccoc1)NC(=O)c1nsnc1-c1ccncn1]

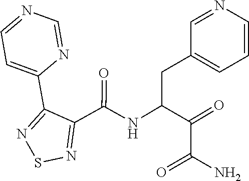 [SMILES: NC(=O)C(=O)C(Cc1cccnc1)NC(=O)c1nsnc1-c1ccncn1]

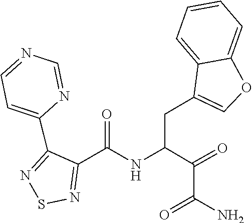 [SMILES: NC(=O)C(=O)C(Cc1coc2ccccc12)NC(=O)c1nsnc1-c1ccncn1]

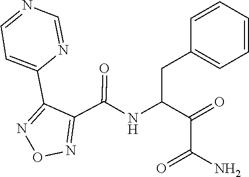 [SMILES: NC(=O)C(=O)C(Cc1ccccc1)NC(=O)c1nonc1-c1ccncn1]

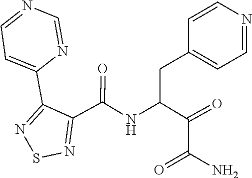 [SMILES: NC(=O)C(=O)C(Cc1ccncc1)NC(=O)c1nsnc1-c1ccncn1]

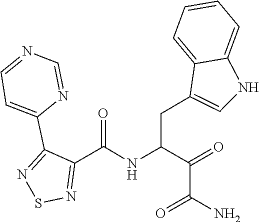 [SMILES: NC(=O)C(=O)C(Cc1c[nH]c2ccccc12)NC(=O)c1nsnc1-c1ccncn1]

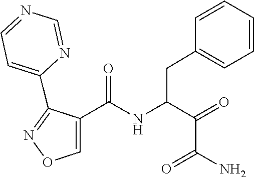 [SMILES: NC(=O)C(=O)C(Cc1ccccc1)NC(=O)c1conc1-c1ccncn1]

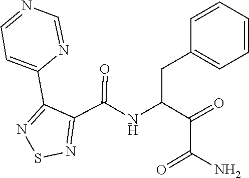 [SMILES: NC(=O)C(=O)C(Cc1ccccc1)NC(=O)c1nsnc1-c1ccncn1]

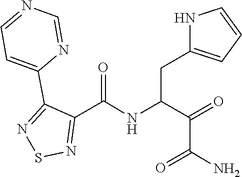 [SMILES: NC(=O)C(=O)C(Cc1ccc[nH]1)NC(=O)c1nsnc1-c1ccncn1]

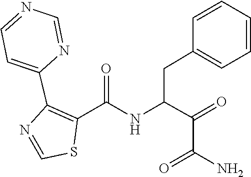 [SMILES: NC(=O)C(=O)C(Cc1ccccc1)NC(=O)c1scnc1-c1ccncn1]

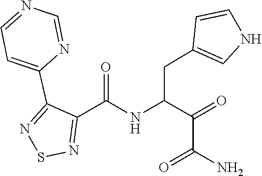 [SMILES: NC(=O)C(=O)C(Cc1cc[nH]c1)NC(=O)c1nsnc1-c1ccncn1]

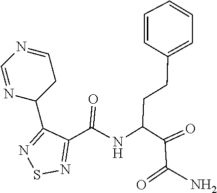 [SMILES: NC(=O)C(=O)C(CCc1ccccc1)NC(=O)c1nsnc1C1CC=NC=N1]

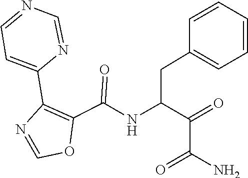 [SMILES: NC(=O)C(=O)C(Cc1ccccc1)NC(=O)c1ocnc1-c1ccncn1]

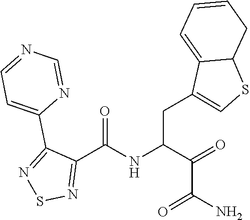 [SMILES: NC(=O)C(=O)C(CC1=CSC2CC=CC=C12)NC(=O)c1nsnc1-c1ccncn1]

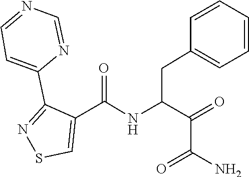 [SMILES: NC(=O)C(=O)C(Cc1ccccc1)NC(=O)c1csnc1-c1ccncn1]

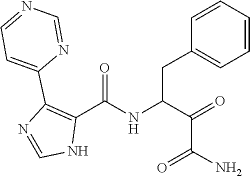 [SMILES: NC(=O)C(=O)C(Cc1ccccc1)NC(=O)c1[nH]cnc1-c1ccncn1]

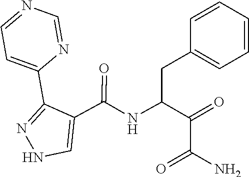 [SMILES: NC(=O)C(=O)C(Cc1ccccc1)NC(=O)c1c[nH]nc1-c1ccncn1]

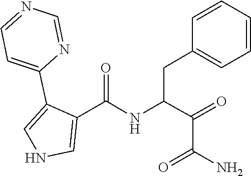 [SMILES: NC(=O)C(=O)C(Cc1ccccc1)NC(=O)c1c[nH]cc1-c1ccncn1]

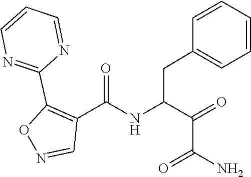 [SMILES: NC(=O)C(=O)C(Cc1ccccc1)NC(=O)c1cnoc1-c1ncccn1]

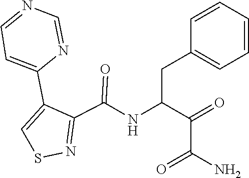 [SMILES: NC(=O)C(=O)C(Cc1ccccc1)NC(=O)c1nscc1-c1ccncn1]

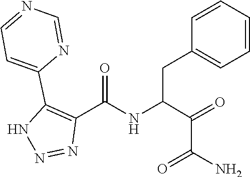 [SMILES: NC(=O)C(=O)C(Cc1ccccc1)NC(=O)c1nn[nH]c1-c1ccncn1]

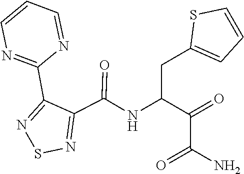 [SMILES: NC(=O)C(=O)C(Cc1cccs1)NC(=O)c1nsnc1-c1ncccn1]

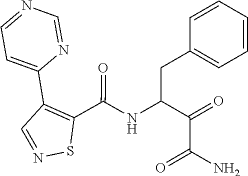 [SMILES: NC(=O)C(=O)C(Cc1ccccc1)NC(=O)c1sncc1-c1ccncn1]

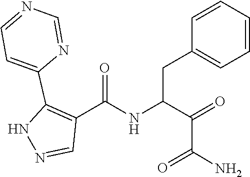 [SMILES: NC(=O)C(=O)C(Cc1ccccc1)NC(=O)c1cn[nH]c1-c1ccncn1]

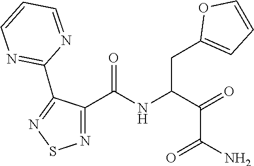 [SMILES: NC(=O)C(=O)C(Cc1ccco1)NC(=O)c1nsnc1-c1ncccn1]

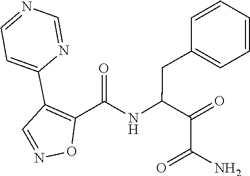 [SMILES: NC(=O)C(=O)C(Cc1ccccc1)NC(=O)c1oncc1-c1ccncn1]

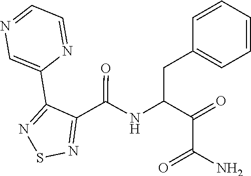 [SMILES: NC(=O)C(=O)C(Cc1ccccc1)NC(=O)c1nsnc1-c1cnccn1]

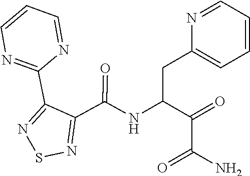 [SMILES: NC(=O)C(=O)C(Cc1ccccn1)NC(=O)c1nsnc1-c1ncccn1]

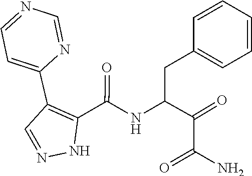 [SMILES: NC(=O)C(=O)C(Cc1ccccc1)NC(=O)c1[nH]ncc1-c1ccncn1]

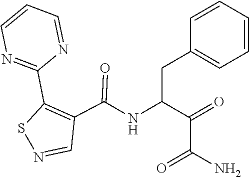 [SMILES: NC(=O)C(=O)C(Cc1ccccc1)NC(=O)c1cnsc1-c1ncccn1]

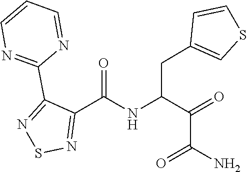 [SMILES: NC(=O)C(=O)C(Cc1ccsc1)NC(=O)c1nsnc1-c1ncccn1]

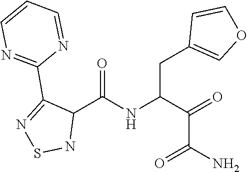

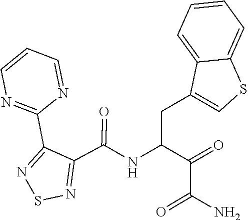 [SMILES: NC(=O)C(=O)C(Cc1csc2ccccc12)NC(=O)c1nsnc1-c1ncccn1]

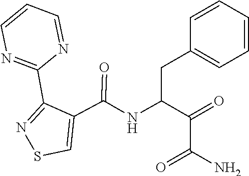 [SMILES: NC(=O)C(=O)C(Cc1ccccc1)NC(=O)c1csnc1-c1ncccn1]

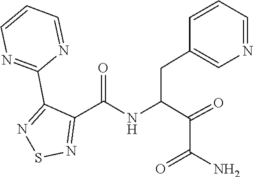 [SMILES: NC(=O)C(=O)C(Cc1cccnc1)NC(=O)c1nsnc1-c1ncccn1]

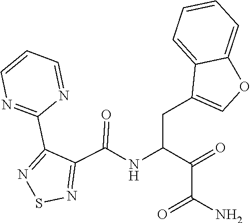 [SMILES: NC(=O)C(=O)C(Cc1coc2ccccc12)NC(=O)c1nsnc1-c1ncccn1]

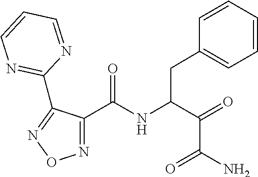 [SMILES: NC(=O)C(=O)C(Cc1ccccc1)NC(=O)c1nonc1-c1ncccn1]

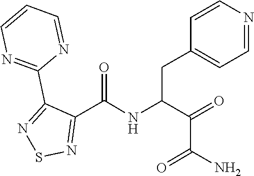 [SMILES: NC(=O)C(=O)C(Cc1ccncc1)NC(=O)c1nsnc1-c1ncccn1]

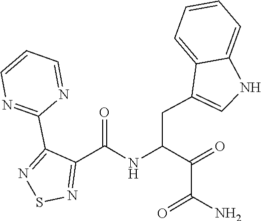 [SMILES: NC(=O)C(=O)C(Cc1c[nH]c2ccccc12)NC(=O)c1nsnc1-c1ncccn1]

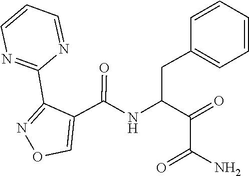 [SMILES: NC(=O)C(=O)C(Cc1ccccc1)NC(=O)c1conc1-c1ncccn1]

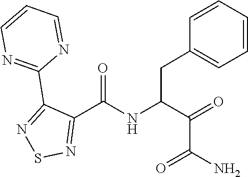 [SMILES: NC(=O)C(=O)C(Cc1ccccc1)NC(=O)c1nsnc1-c1ncccn1]

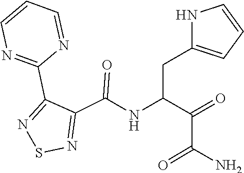 [SMILES: NC(=O)C(=O)C(Cc1ccc[nH]1)NC(=O)c1nsnc1-c1ncccn1]

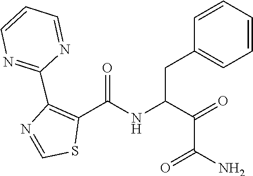 [SMILES: NC(=O)C(=O)C(Cc1ccccc1)NC(=O)c1scnc1-c1ncccn1]

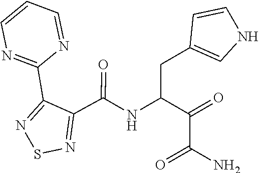 [SMILES: NC(=O)C(=O)C(Cc1cc[nH]c1)NC(=O)c1nsnc1-c1ncccn1]

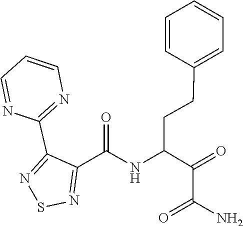 [SMILES: NC(=O)C(=O)C(CCc1ccccc1)NC(=O)c1nsnc1-c1ncccn1]

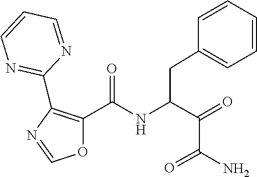 [SMILES: NC(=O)C(=O)C(Cc1ccccc1)NC(=O)c1ocnc1-c1ncccn1]

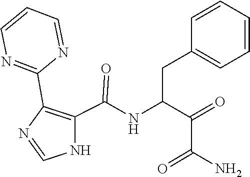 [SMILES: NC(=O)C(=O)C(Cc1ccccc1)NC(=O)c1[nH]cnc1-c1ncccn1]

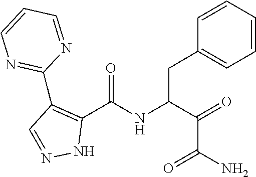 [SMILES: NC(=O)C(=O)C(Cc1ccccc1)NC(=O)c1[nH]ncc1-c1ncccn1]

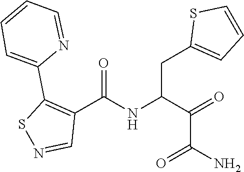 [SMILES: NC(=O)C(=O)C(Cc1cccs1)NC(=O)c1cnsc1-c1ccccn1]

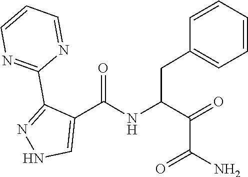 [SMILES: NC(=O)C(=O)C(Cc1ccccc1)NC(=O)c1c[nH]nc1-c1ncccn1]

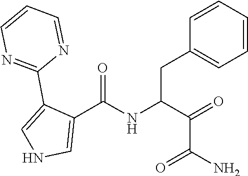 [SMILES: NC(=O)C(=O)C(Cc1ccccc1)NC(=O)c1c[nH]cc1-c1ncccn1]

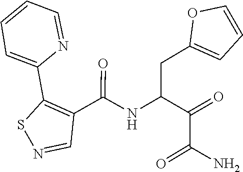 [SMILES: NC(=O)C(=O)C(Cc1ccco1)NC(=O)c1cnsc1-c1ccccn1]

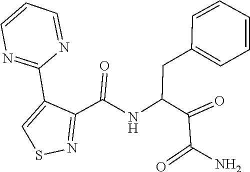 [SMILES: NC(=O)C(=O)C(Cc1ccccc1)NC(=O)c1nscc1-c1ncccn1]

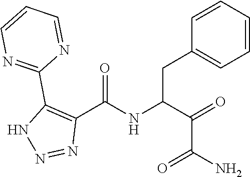 [SMILES: NC(=O)C(=O)C(Cc1ccccc1)NC(=O)c1nn[nH]c1-c1ncccn1]

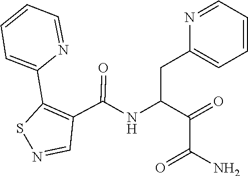 [SMILES: NC(=O)C(=O)C(Cc1ccccn1)NC(=O)c1cnsc1-c1ccccn1]

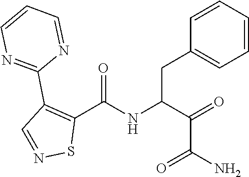 [SMILES: NC(=O)C(=O)C(Cc1ccccc1)NC(=O)c1sncc1-c1ncccn1]

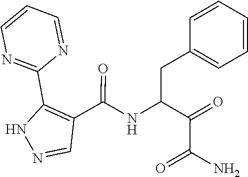 [SMILES: NC(=O)C(=O)C(Cc1ccccc1)NC(=O)c1cn[nH]c1-c1ncccn1]

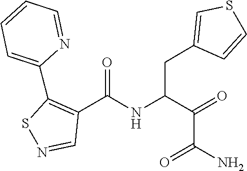 [SMILES: NC(=O)C(=O)C(Cc1ccsc1)NC(=O)c1cnsc1-c1ccccn1]

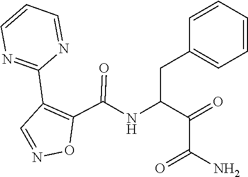 [SMILES: NC(=O)C(=O)C(Cc1ccccc1)NC(=O)c1oncc1-c1ncccn1]

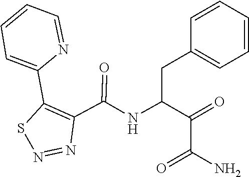 [SMILES: NC(=O)C(=O)C(Cc1ccccc1)NC(=O)c1nnsc1-c1ccccn1]

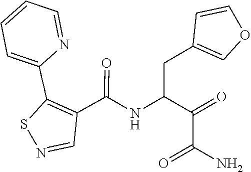 [SMILES: NC(=O)C(=O)C(Cc1ccoc1)NC(=O)c1cnsc1-c1ccccn1]

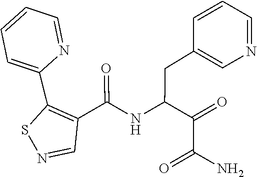 [SMILES: NC(=O)C(=O)C(Cc1cccnc1)NC(=O)c1cnsc1-c1ccccn1]

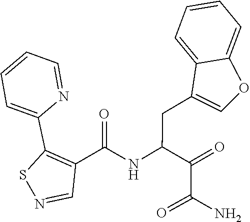 [SMILES: NC(=O)C(=O)C(Cc1coc2ccccc12)NC(=O)c1cnsc1-c1ccccn1]

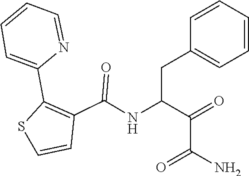 [SMILES: NC(=O)C(=O)C(Cc1ccccc1)NC(=O)c1ccsc1-c1ccccn1]

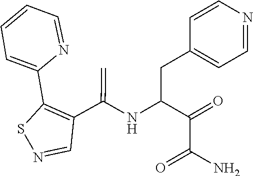 [SMILES: C=C(NC(Cc1ccncc1)C(=O)C(N)=O)c1cnsc1-c1ccccn1]

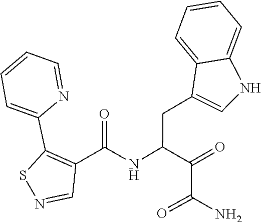 [SMILES: NC(=O)C(=O)C(Cc1c[nH]c2ccccc12)NC(=O)c1cnsc1-c1ccccn1]

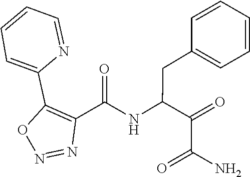 [SMILES: NC(=O)C(=O)C(Cc1ccccc1)NC(=O)c1nnoc1-c1ccccn1]

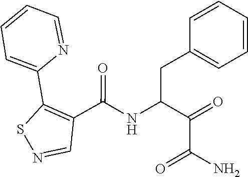 [SMILES: NC(=O)C(=O)C(Cc1ccccc1)NC(=O)c1cnsc1-c1ccccn1]

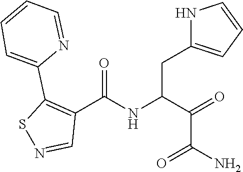 [SMILES: NC(=O)C(=O)C(Cc1ccc[nH]1)NC(=O)c1cnsc1-c1ccccn1]

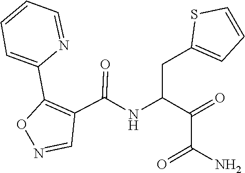 [SMILES: NC(=O)C(=O)C(Cc1cccs1)NC(=O)c1cnoc1-c1ccccn1]

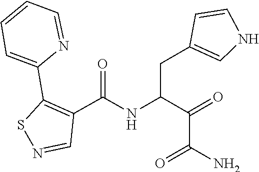 [SMILES: NC(=O)C(=O)C(Cc1cc[nH]c1)NC(=O)c1cnsc1-c1ccccn1]

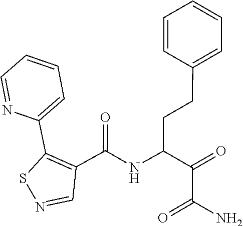 [SMILES: NC(=O)C(=O)C(CCc1ccccc1)NC(=O)c1cnsc1-c1ccccn1]

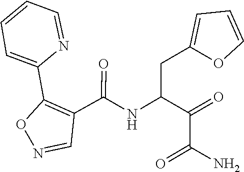 [SMILES: NC(=O)C(=O)C(Cc1ccco1)NC(=O)c1cnoc1-c1ccccn1]

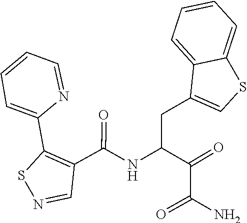 [SMILES: NC(=O)C(=O)C(Cc1csc2ccccc12)NC(=O)c1cnsc1-c1ccccn1]

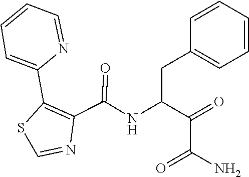 [SMILES: NC(=O)C(=O)C(Cc1ccccc1)NC(=O)c1ncsc1-c1ccccn1]

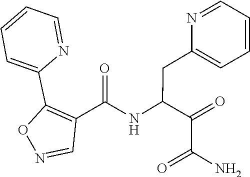 [SMILES: NC(=O)C(=O)C(Cc1ccccn1)NC(=O)c1cnoc1-c1ccccn1]

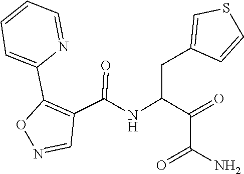 [SMILES: NC(=O)C(=O)C(Cc1ccsc1)NC(=O)c1cnoc1-c1ccccn1]

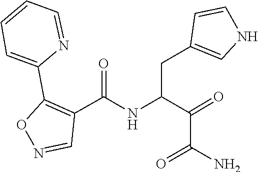 [SMILES: NC(=O)C(=O)C(Cc1cc[nH]c1)NC(=O)c1cnoc1-c1ccccn1]

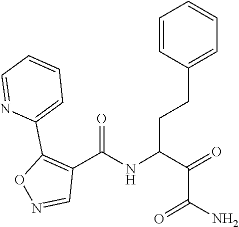 [SMILES: NC(=O)C(=O)C(CCc1ccccc1)NC(=O)c1cnoc1-c1ccccn1]

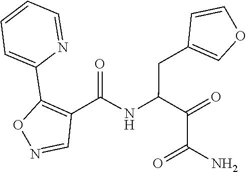 [SMILES: NC(=O)C(=O)C(Cc1ccoc1)NC(=O)c1cnoc1-c1ccccn1]

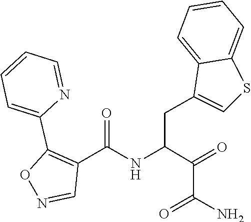 [SMILES: NC(=O)C(=O)C(Cc1csc2ccccc12)NC(=O)c1cnoc1-c1ccccn1]

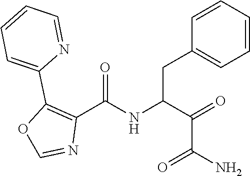 [SMILES: NC(=O)C(=O)C(Cc1ccccc1)NC(=O)c1ncoc1-c1ccccn1]

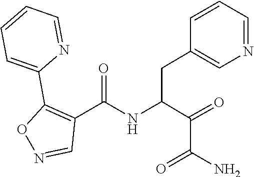 [SMILES: NC(=O)C(=O)C(Cc1cccnc1)NC(=O)c1cnoc1-c1ccccn1]

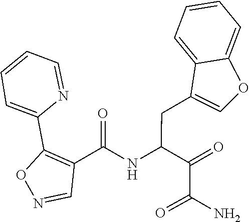 [SMILES: NC(=O)C(=O)C(Cc1coc2ccccc12)NC(=O)c1cnoc1-c1ccccn1]

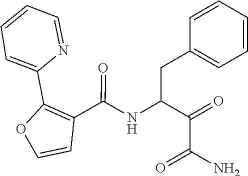 [SMILES: NC(=O)C(=O)C(Cc1ccccc1)NC(=O)c1ccoc1-c1ccccn1]

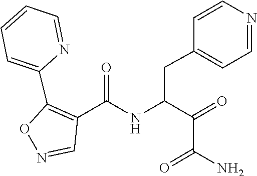 [SMILES: NC(=O)C(=O)C(Cc1ccncc1)NC(=O)c1cnoc1-c1ccccn1]

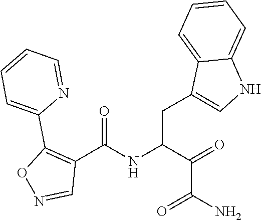 [SMILES: NC(=O)C(=O)C(Cc1c[nH]c2ccccc12)NC(=O)c1cnoc1-c1ccccn1]

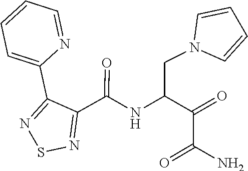 [SMILES: NC(=O)C(=O)C(Cn1cccc1)NC(=O)c1nsnc1-c1ccccn1]

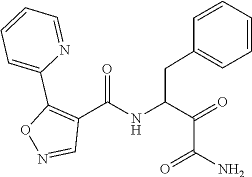 [SMILES: NC(=O)C(=O)C(Cc1ccccc1)NC(=O)c1cnoc1-c1ccccn1]

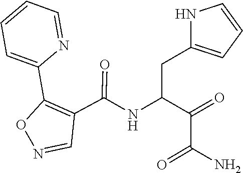 [SMILES: NC(=O)C(=O)C(Cc1ccc[nH]1)NC(=O)c1cnoc1-c1ccccn1]

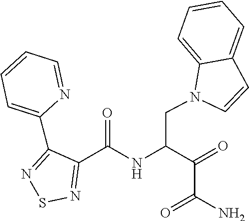 [SMILES: NC(=O)C(=O)C(Cn1ccc2ccccc21)NC(=O)c1nsnc1-c1ccccn1]

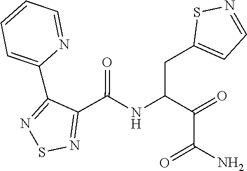 [SMILES: NC(=O)C(=O)C(Cc1ccns1)NC(=O)c1nsnc1-c1ccccn1]

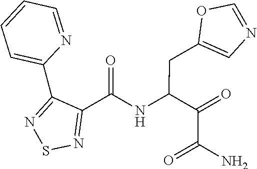 [SMILES: NC(=O)C(=O)C(Cc1cnco1)NC(=O)c1nsnc1-c1ccccn1]

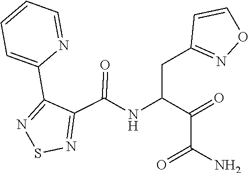 [SMILES: NC(=O)C(=O)C(Cc1ccon1)NC(=O)c1nsnc1-c1ccccn1]

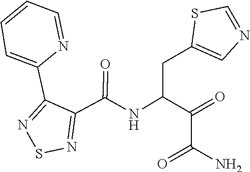 [SMILES: NC(=O)C(=O)C(Cc1cncs1)NC(=O)c1nsnc1-c1ccccn1]

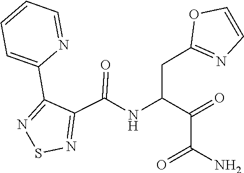 [SMILES: NC(=O)C(=O)C(Cc1ncco1)NC(=O)c1nsnc1-c1ccccn1]

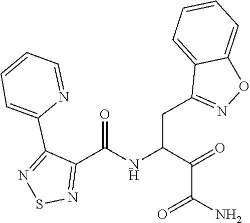 [SMILES: NC(=O)C(=O)C(Cc1noc2ccccc12)NC(=O)c1nsnc1-c1ccccn1]

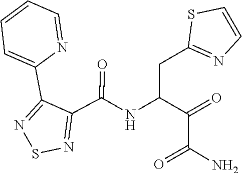 [SMILES: NC(=O)C(=O)C(Cc1nccs1)NC(=O)c1nsnc1-c1ccccn1]

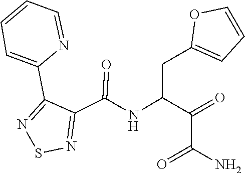 [SMILES: NC(=O)C(=O)C(Cc1ccco1)NC(=O)c1nsnc1-c1ccccn1]

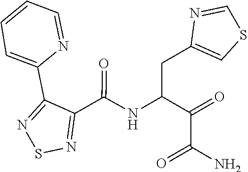 [SMILES: NC(=O)C(=O)C(Cc1cscn1)NC(=O)c1nsnc1-c1ccccn1]

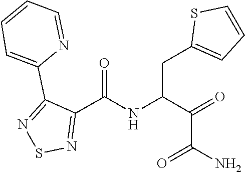 [SMILES: NC(=O)C(=O)C(Cc1cccs1)NC(=O)c1nsnc1-c1ccccn1]

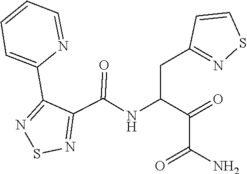 [SMILES: NC(=O)C(=O)C(Cc1ccsn1)NC(=O)c1nsnc1-c1ccccn1]

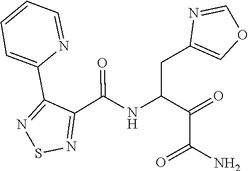 [SMILES: NC(=O)C(=O)C(Cc1cocn1)NC(=O)c1nsnc1-c1ccccn1]

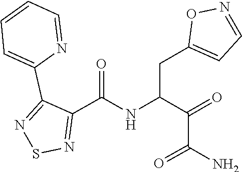 [SMILES: NC(=O)C(=O)C(Cc1ccno1)NC(=O)c1nsnc1-c1ccccn1]

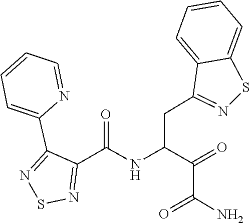 [SMILES: NC(=O)C(=O)C(Cc1nsc2ccccc12)NC(=O)c1nsnc1-c1ccccn1]

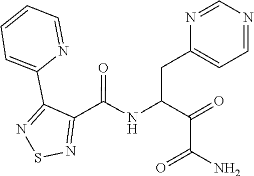 [SMILES: NC(=O)C(=O)C(Cc1ccncn1)NC(=O)c1nsnc1-c1ccccn1]

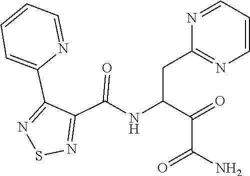 [SMILES: NC(=O)C(=O)C(Cc1ncccn1)NC(=O)c1nsnc1-c1ccccn1]

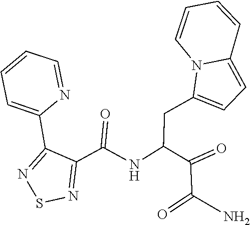 [SMILES: NC(=O)C(=O)C(Cc1ccc2ccccn12)NC(=O)c1nsnc1-c1ccccn1]

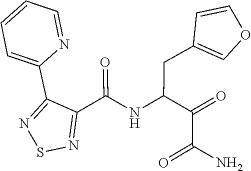 [SMILES: NC(=O)C(=O)C(Cc1ccoc1)NC(=O)c1nsnc1-c1ccccn1]

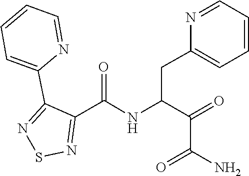 [SMILES: NC(=O)C(=O)C(Cc1ccccn1)NC(=O)c1nsnc1-c1ccccn1]

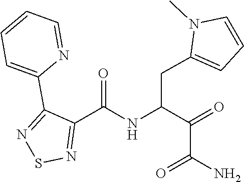 [SMILES: Cn1cccc1CC(NC(=O)c1nsnc1-c1ccccn1)C(=O)C(N)=O]

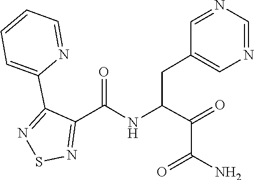 [SMILES: NC(=O)C(=O)C(Cc1cncnc1)NC(=O)c1nsnc1-c1ccccn1]

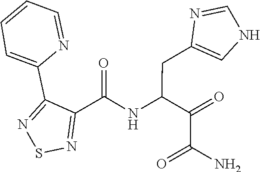 [SMILES: NC(=O)C(=O)C(Cc1c[nH]cn1)NC(=O)c1nsnc1-c1ccccn1]

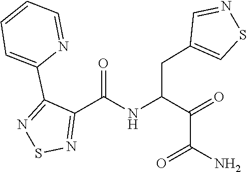 [SMILES: NC(=O)C(=O)C(Cc1cnsc1)NC(=O)c1nsnc1-c1ccccn1]

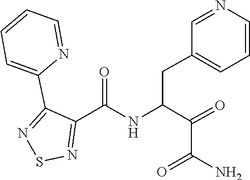 [SMILES: NC(=O)C(=O)C(Cc1cccnc1)NC(=O)c1nsnc1-c1ccccn1]

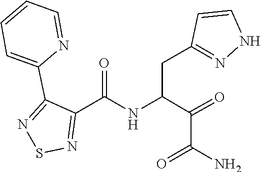 [SMILES: NC(=O)C(=O)C(Cc1cc[nH]n1)NC(=O)c1nsnc1-c1ccccn1]

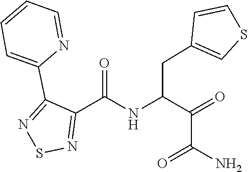 [SMILES: NC(=O)C(=O)C(Cc1ccsc1)NC(=O)c1nsnc1-c1ccccn1]

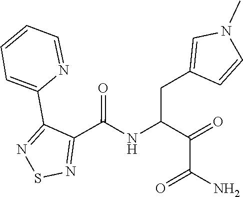 [SMILES: Cn1ccc(CC(NC(=O)c2nsnc2-c2ccccn2)C(=O)C(N)=O)c1]

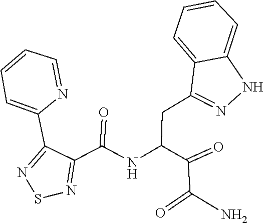 [SMILES: NC(=O)C(=O)C(Cc1n[nH]c2ccccc12)NC(=O)c1nsnc1-c1ccccn1]

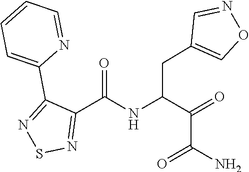 [SMILES: NC(=O)C(=O)C(Cc1cnoc1)NC(=O)c1nsnc1-c1ccccn1]

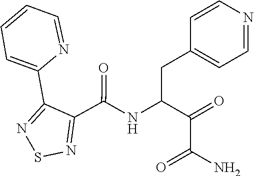 [SMILES: NC(=O)C(=O)C(Cc1ccncc1)NC(=O)c1nsnc1-c1ccccn1]

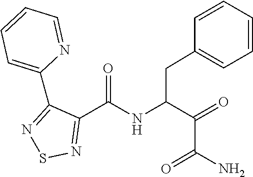 [SMILES: NC(=O)C(=O)C(Cc1ccccc1)NC(=O)c1nsnc1-c1ccccn1]

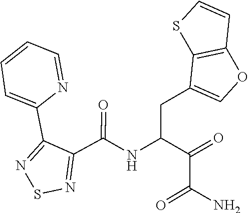 [SMILES: NC(=O)C(=O)C(Cc1coc2ccsc12)NC(=O)c1nsnc1-c1ccccn1]

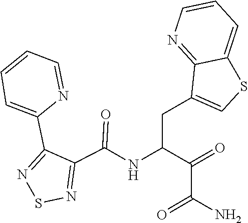 [SMILES: NC(=O)C(=O)C(Cc1csc2cccnc12)NC(=O)c1nsnc1-c1ccccn1]

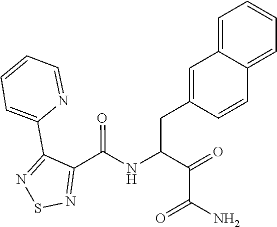 [SMILES: NC(=O)C(=O)C(Cc1ccc2ccccc2c1)NC(=O)c1nsnc1-c1ccccn1]

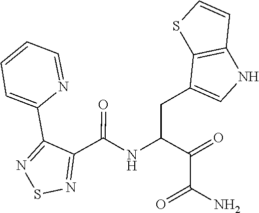 [SMILES: NC(=O)C(=O)C(Cc1c[nH]c2ccsc12)NC(=O)c1nsnc1-c1ccccn1]

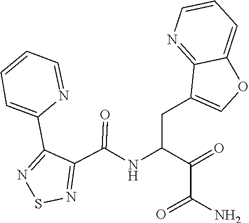 [SMILES: NC(=O)C(=O)C(Cc1coc2cccnc12)NC(=O)c1nsnc1-c1ccccn1]

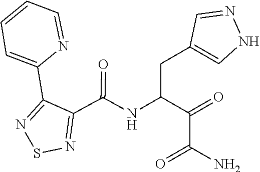 [SMILES: NC(=O)C(=O)C(Cc1cn[nH]c1)NC(=O)c1nsnc1-c1ccccn1]

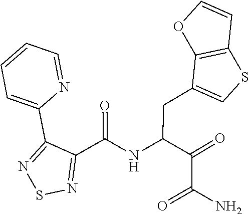 [SMILES: NC(=O)C(=O)C(Cc1csc2ccoc12)NC(=O)c1nsnc1-c1ccccn1]

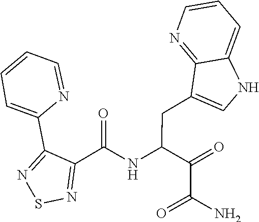 [SMILES: NC(=O)C(=O)C(Cc1c[nH]c2cccnc12)NC(=O)c1nsnc1-c1ccccn1]

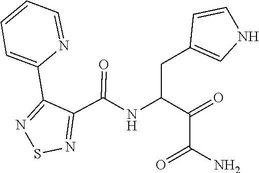 [SMILES: NC(=O)C(=O)C(Cc1cc[nH]c1)NC(=O)c1nsnc1-c1ccccn1]

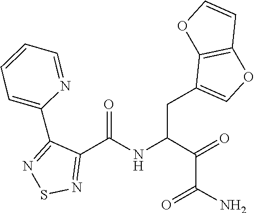 [SMILES: NC(=O)C(=O)C(Cc1coc2ccoc12)NC(=O)c1nsnc1-c1ccccn1]

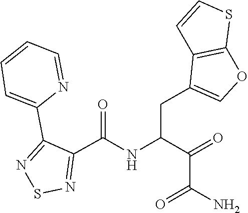 [SMILES: NC(=O)C(=O)C(Cc1coc2sccc12)NC(=O)c1nsnc1-c1ccccn1]

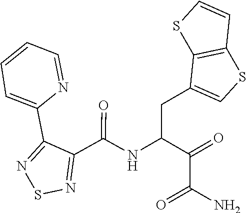 [SMILES: NC(=O)C(=O)C(Cc1csc2ccsc12)NC(=O)c1nsnc1-c1ccccn1]

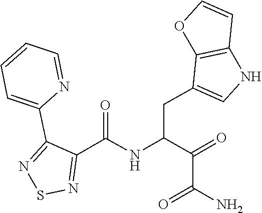 [SMILES: NC(=O)C(=O)C(Cc1c[nH]c2ccoc12)NC(=O)c1nsnc1-c1ccccn1]

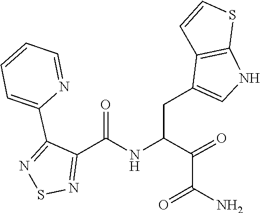 [SMILES: NC(=O)C(=O)C(Cc1c[nH]c2sccc12)NC(=O)c1nsnc1-c1ccccn1]

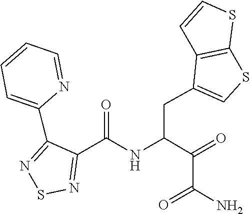 [SMILES: NC(=O)C(=O)C(Cc1csc2sccc12)NC(=O)c1nsnc1-c1ccccn1]

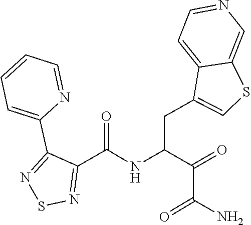 [SMILES: NC(=O)C(=O)C(Cc1csc2cnccc12)NC(=O)c1nsnc1-c1ccccn1]

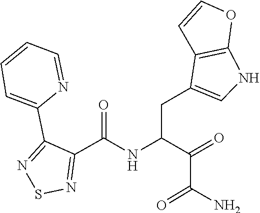 [SMILES: NC(=O)C(=O)C(Cc1c[nH]c2occc12)NC(=O)c1nsnc1-c1ccccn1]

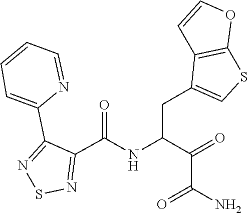 [SMILES: NC(=O)C(=O)C(Cc1csc2occc12)NC(=O)c1nsnc1-c1ccccn1]

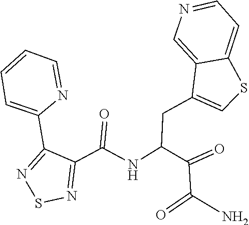 [SMILES: NC(=O)C(=O)C(Cc1csc2ccncc12)NC(=O)c1nsnc1-c1ccccn1]

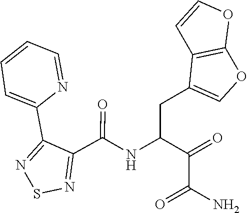 [SMILES: NC(=O)C(=O)C(Cc1coc2occc12)NC(=O)c1nsnc1-c1ccccn1]

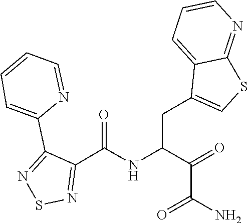 [SMILES: NC(=O)C(=O)C(Cc1csc2ncccc12)NC(=O)c1nsnc1-c1ccccn1]

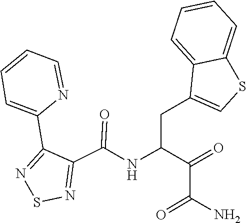 [SMILES: NC(=O)C(=O)C(Cc1csc2ccccc12)NC(=O)c1nsnc1-c1ccccn1]

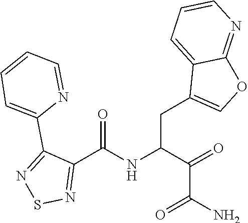 [SMILES: NC(=O)C(=O)C(Cc1coc2ncccc12)NC(=O)c1nsnc1-c1ccccn1]

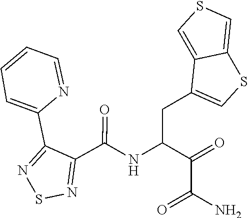 [SMILES: NC(=O)C(=O)C(Cc1csc2cscc12)NC(=O)c1nsnc1-c1ccccn1]

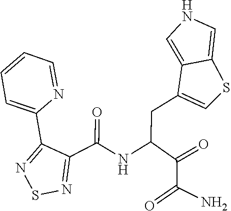 [SMILES: NC(=O)C(=O)C(Cc1csc2c[nH]cc12)NC(=O)c1nsnc1-c1ccccn1]

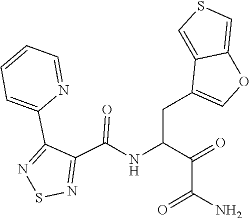 [SMILES: NC(=O)C(=O)C(Cc1coc2cscc12)NC(=O)c1nsnc1-c1ccccn1]

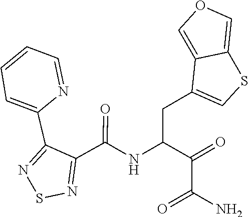 [SMILES: NC(=O)C(=O)C(Cc1csc2cocc12)NC(=O)c1nsnc1-c1ccccn1]

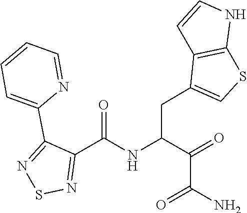 [SMILES: NC(=O)C(=O)C(Cc1csc2[nH]ccc12)NC(=O)c1nsnc1-c1ccccn1]

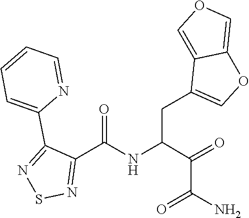 [SMILES: NC(=O)C(=O)C(Cc1coc2cocc12)NC(=O)c1nsnc1-c1ccccn1]

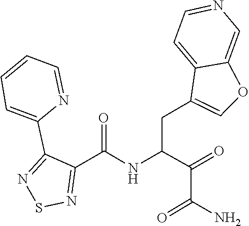 [SMILES: NC(=O)C(=O)C(Cc1coc2cnccc12)NC(=O)c1nsnc1-c1ccccn1]

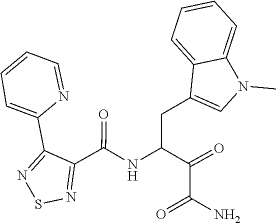 [SMILES: Cn1cc(CC(NC(=O)c2nsnc2-c2ccccn2)C(=O)C(N)=O)c2ccccc21]

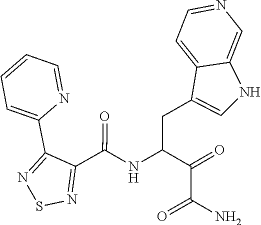 [SMILES: NC(=O)C(=O)C(Cc1c[nH]c2cnccc12)NC(=O)c1nsnc1-c1ccccn1]

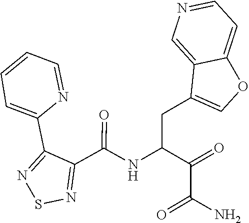 [SMILES: NC(=O)C(=O)C(Cc1coc2ccncc12)NC(=O)c1nsnc1-c1ccccn1]

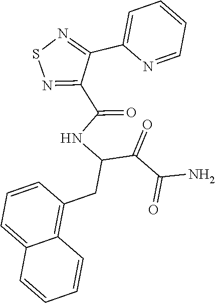 [SMILES: NC(=O)C(=O)C(Cc1cccc2ccccc12)NC(=O)c1nsnc1-c1ccccn1]

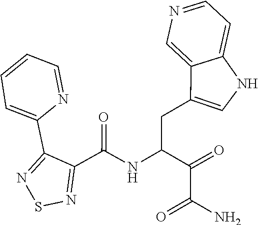 [SMILES: NC(=O)C(=O)C(Cc1c[nH]c2ccncc12)NC(=O)c1nsnc1-c1ccccn1]

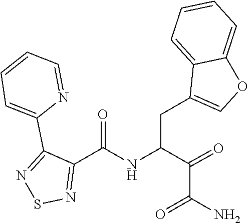 [SMILES: NC(=O)C(=O)C(Cc1coc2ccccc12)NC(=O)c1nsnc1-c1ccccn1]

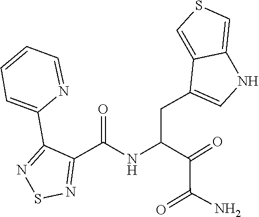 [SMILES: NC(=O)C(=O)C(Cc1c[nH]c2cscc12)NC(=O)c1nsnc1-c1ccccn1]

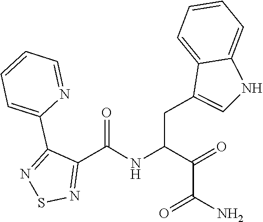 [SMILES: NC(=O)C(=O)C(Cc1c[nH]c2ccccc12)NC(=O)c1nsnc1-c1ccccn1]

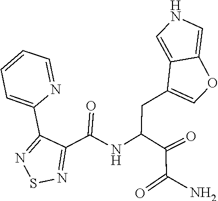 [SMILES: NC(=O)C(=O)C(Cc1coc2c[nH]cc12)NC(=O)c1nsnc1-c1ccccn1]

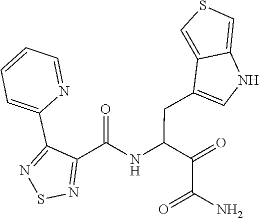 [SMILES: NC(=O)C(=O)C(Cc1c[nH]c2cscc12)NC(=O)c1nsnc1-c1ccccn1]

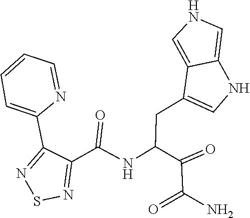 [SMILES: NC(=O)C(=O)C(Cc1c[nH]c2c[nH]cc12)NC(=O)c1nsnc1-c1ccccn1]

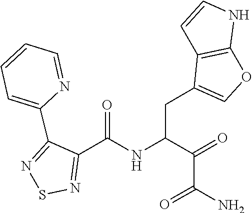 [SMILES: NC(=O)C(=O)C(Cc1coc2[nH]ccc12)NC(=O)c1nsnc1-c1ccccn1]

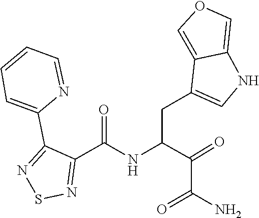 [SMILES: NC(=O)C(=O)C(Cc1c[nH]c2cocc12)NC(=O)c1nsnc1-c1ccccn1]

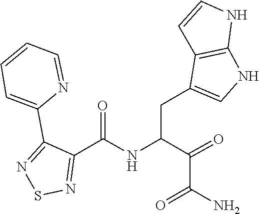 [SMILES: NC(=O)C(=O)C(Cc1c[nH]c2[nH]ccc12)NC(=O)c1nsnc1-c1ccccn1]

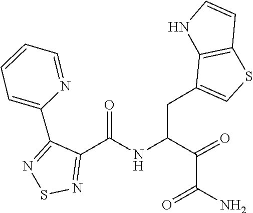 [SMILES: NC(=O)C(=O)C(Cc1csc2cc[nH]c12)NC(=O)c1nsnc1-c1ccccn1]

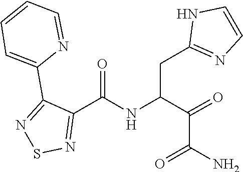 [SMILES: NC(=O)C(=O)C(Cc1ncc[nH]1)NC(=O)c1nsnc1-c1ccccn1]

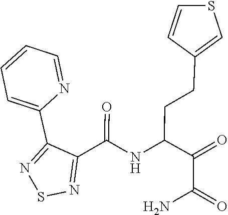 [SMILES: NC(=O)C(=O)C(CCc1ccsc1)NC(=O)c1nsnc1-c1ccccn1]

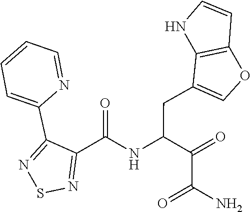 [SMILES: NC(=O)C(=O)C(Cc1coc2cc[nH]c12)NC(=O)c1nsnc1-c1ccccn1]

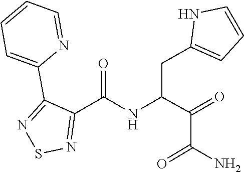 [SMILES: NC(=O)C(=O)C(Cc1ccc[nH]1)NC(=O)c1nsnc1-c1ccccn1]

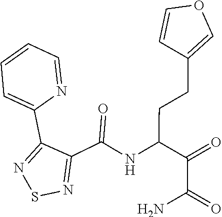 [SMILES: NC(=O)C(=O)C(CCc1ccoc1)NC(=O)c1nsnc1-c1ccccn1]

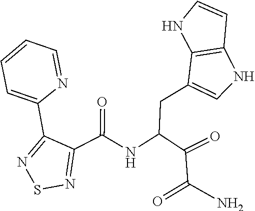 [SMILES: NC(=O)C(=O)C(Cc1c[nH]c2cc[nH]c12)NC(=O)c1nsnc1-c1ccccn1]

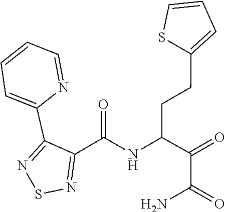 [SMILES: NC(=O)C(=O)C(CCc1cccs1)NC(=O)c1nsnc1-c1ccccn1]

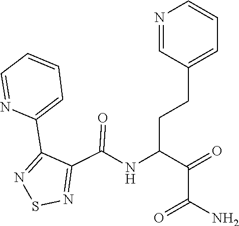 [SMILES: NC(=O)C(=O)C(CCc1cccnc1)NC(=O)c1nsnc1-c1ccccn1]

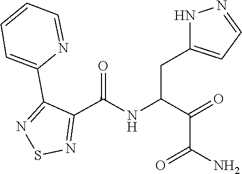 [SMILES: NC(=O)C(=O)C(Cc1ccn[nH]1)NC(=O)c1nsnc1-c1ccccn1]

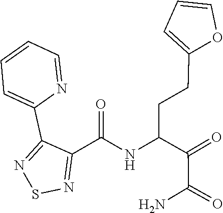 [SMILES: NC(=O)C(=O)C(CCc1ccco1)NC(=O)c1nsnc1-c1ccccn1]

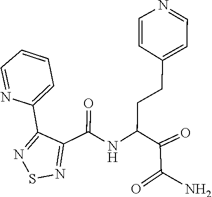 [SMILES: NC(=O)C(=O)C(CCc1ccncc1)NC(=O)c1nsnc1-c1ccccn1]

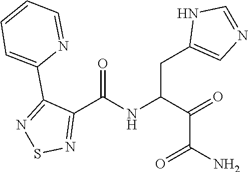 [SMILES: NC(=O)C(=O)C(Cc1cnc[nH]1)NC(=O)c1nsnc1-c1ccccn1]

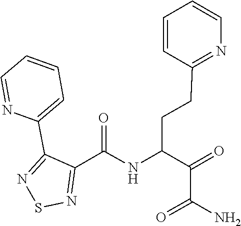 [SMILES: NC(=O)C(=O)C(CCc1ccccn1)NC(=O)c1nsnc1-c1ccccn1]

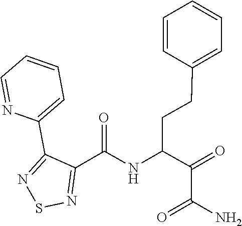 [SMILES: NC(=O)C(=O)C(CCc1ccccc1)NC(=O)c1nsnc1-c1ccccn1]

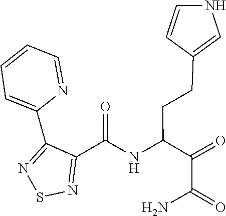 [SMILES: NC(=O)C(=O)C(CCc1cc[nH]c1)NC(=O)c1nsnc1-c1ccccn1]

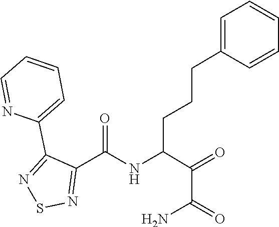 [SMILES: NC(=O)C(=O)C(CCCc1ccccc1)NC(=O)c1nsnc1-c1ccccn1]

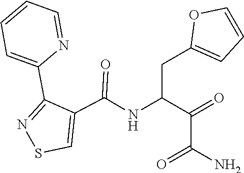 [SMILES: NC(=O)C(=O)C(Cc1ccco1)NC(=O)c1csnc1-c1ccccn1]

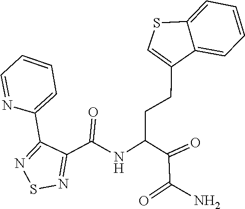 [SMILES: NC(=O)C(=O)C(CCc1csc2ccccc12)NC(=O)c1nsnc1-c1ccccn1]

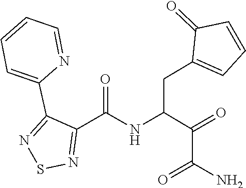 [SMILES: NC(=O)C(=O)C(CC1=CC=CC1=O)NC(=O)c1nsnc1-c1ccccn1]

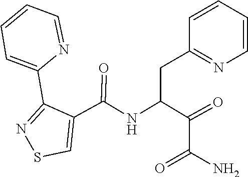 [SMILES: NC(=O)C(=O)C(Cc1ccccn1)NC(=O)c1csnc1-c1ccccn1]

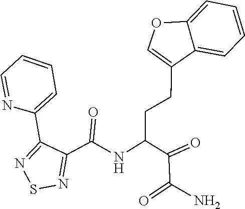 [SMILES: NC(=O)C(=O)C(CCc1coc2ccccc12)NC(=O)c1nsnc1-c1ccccn1]

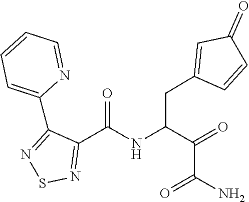 [SMILES: NC(=O)C(=O)C(CC1=CC(=O)C=C1)NC(=O)c1nsnc1-c1ccccn1]

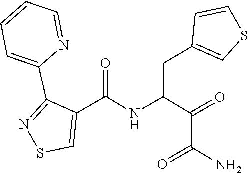 [SMILES: NC(=O)C(=O)C(Cc1ccsc1)NC(=O)c1csnc1-c1ccccn1]

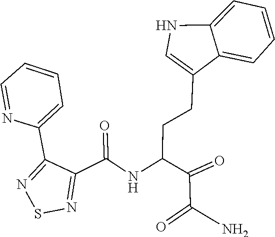 [SMILES: NC(=O)C(=O)C(CCc1c[nH]c2ccccc12)NC(=O)c1nsnc1-c1ccccn1]

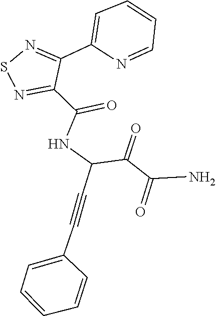 [SMILES: NC(=O)C(=O)C(C#Cc1ccccc1)NC(=O)c1nsnc1-c1ccccn1]

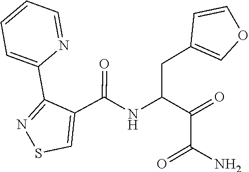 [SMILES: NC(=O)C(=O)C(Cc1ccoc1)NC(=O)c1csnc1-c1ccccn1]

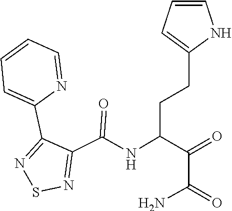 [SMILES: NC(=O)C(=O)C(CCc1ccc[nH]1)NC(=O)c1nsnc1-c1ccccn1]

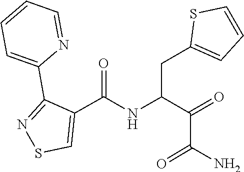 [SMILES: NC(=O)C(=O)C(Cc1cccs1)NC(=O)c1csnc1-c1ccccn1]

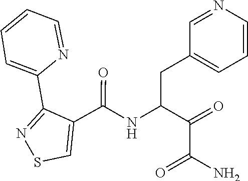 [SMILES: NC(=O)C(=O)C(Cc1cccnc1)NC(=O)c1csnc1-c1ccccn1]

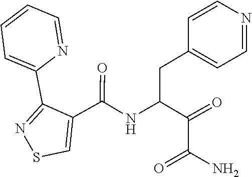 [SMILES: NC(=O)C(=O)C(Cc1ccncc1)NC(=O)c1csnc1-c1ccccn1]

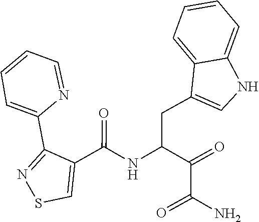 [SMILES: NC(=O)C(=O)C(Cc1c[nH]c2ccccc12)NC(=O)c1csnc1-c1ccccn1]

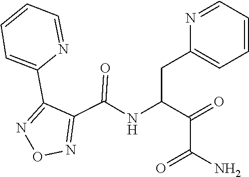 [SMILES: NC(=O)C(=O)C(Cc1ccccn1)NC(=O)c1nonc1-c1ccccn1]

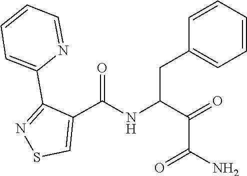 [SMILES: NC(=O)C(=O)C(Cc1ccccc1)NC(=O)c1csnc1-c1ccccn1]

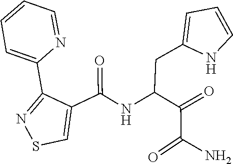 [SMILES: NC(=O)C(=O)C(Cc1ccc[nH]1)NC(=O)c1csnc1-c1ccccn1]

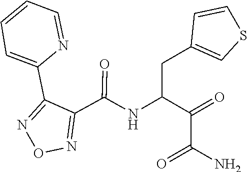 [SMILES: NC(=O)C(=O)C(Cc1ccsc1)NC(=O)c1nonc1-c1ccccn1]

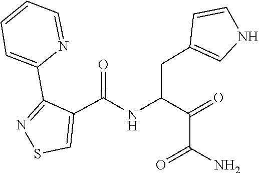 [SMILES: NC(=O)C(=O)C(Cc1cc[nH]c1)NC(=O)c1csnc1-c1ccccn1]

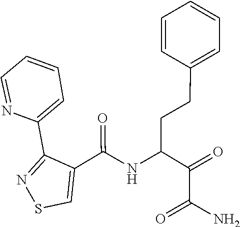 [SMILES: NC(=O)C(=O)C(CCc1ccccc1)NC(=O)c1csnc1-c1ccccn1]

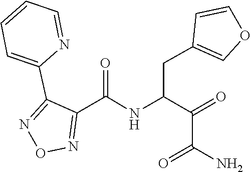 [SMILES: NC(=O)C(=O)C(Cc1ccoc1)NC(=O)c1nonc1-c1ccccn1]

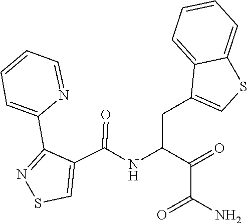 [SMILES: NC(=O)C(=O)C(Cc1csc2ccccc12)NC(=O)c1csnc1-c1ccccn1]

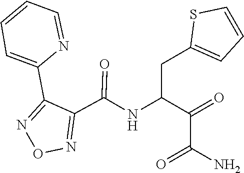 [SMILES: NC(=O)C(=O)C(Cc1cccs1)NC(=O)c1nonc1-c1ccccn1]

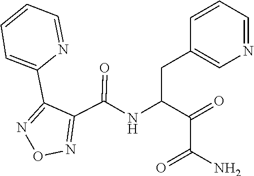 [SMILES: NC(=O)C(=O)C(Cc1cccnc1)NC(=O)c1nonc1-c1ccccn1]

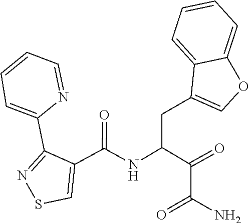 [SMILES: NC(=O)C(=O)C(Cc1coc2ccccc12)NC(=O)c1csnc1-c1ccccn1]

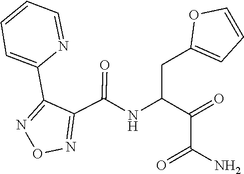 [SMILES: NC(=O)C(=O)C(Cc1ccco1)NC(=O)c1nonc1-c1ccccn1]

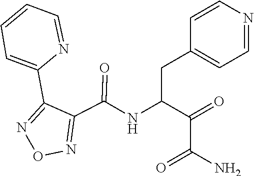 [SMILES: NC(=O)C(=O)C(Cc1ccncc1)NC(=O)c1nonc1-c1ccccn1]

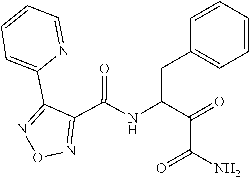 [SMILES: NC(=O)C(=O)C(Cc1ccccc1)NC(=O)c1nonc1-c1ccccn1]

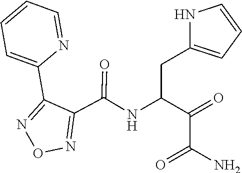 [SMILES: NC(=O)C(=O)C(Cc1ccc[nH]1)NC(=O)c1nonc1-c1ccccn1]

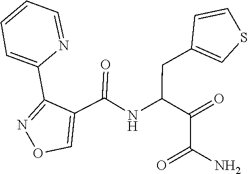 [SMILES: NC(=O)C(=O)C(Cc1ccsc1)NC(=O)c1conc1-c1ccccn1]

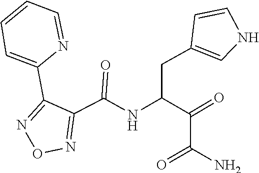 [SMILES: NC(=O)C(=O)C(Cc1cc[nH]c1)NC(=O)c1nonc1-c1ccccn1]

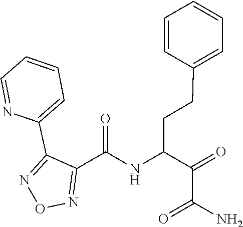 [SMILES: NC(=O)C(=O)C(CCc1ccccc1)NC(=O)c1nonc1-c1ccccn1]

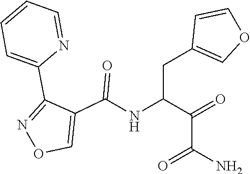 [SMILES: NC(=O)C(=O)C(Cc1ccoc1)NC(=O)c1conc1-c1ccccn1]

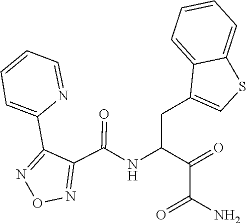 [SMILES: NC(=O)C(=O)C(Cc1csc2ccccc12)NC(=O)c1nonc1-c1ccccn1]

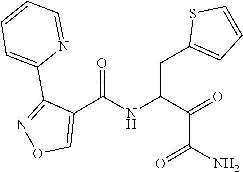 [SMILES: NC(=O)C(=O)C(Cc1cccs1)NC(=O)c1conc1-c1ccccn1]

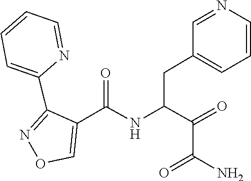 [SMILES: NC(=O)C(=O)C(Cc1cccnc1)NC(=O)c1conc1-c1ccccn1]

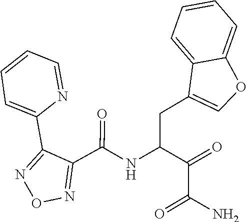 [SMILES: NC(=O)C(=O)C(Cc1coc2ccccc12)NC(=O)c1nonc1-c1ccccn1]

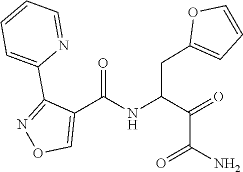 [SMILES: NC(=O)C(=O)C(Cc1ccco1)NC(=O)c1conc1-c1ccccn1]

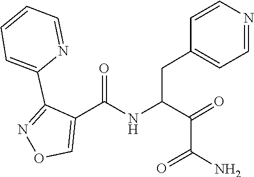 [SMILES: NC(=O)C(=O)C(Cc1ccncc1)NC(=O)c1conc1-c1ccccn1]

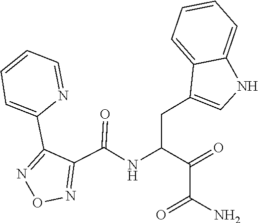 [SMILES: NC(=O)C(=O)C(Cc1c[nH]c2ccccc12)NC(=O)c1nonc1-c1ccccn1]

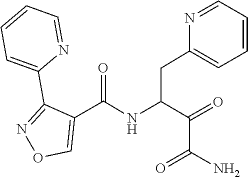 [SMILES: NC(=O)C(=O)C(Cc1ccccn1)NC(=O)c1conc1-c1ccccn1]

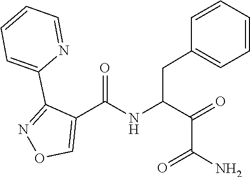 [SMILES: NC(=O)C(=O)C(Cc1ccccc1)NC(=O)c1conc1-c1ccccn1]

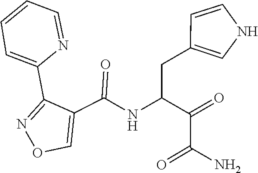 [SMILES: NC(=O)C(=O)C(Cc1cc[nH]c1)NC(=O)c1conc1-c1ccccn1]

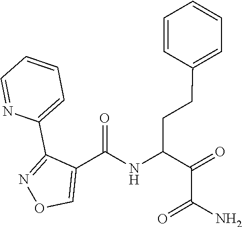 [SMILES: NC(=O)C(=O)C(CCc1ccccc1)NC(=O)c1conc1-c1ccccn1]

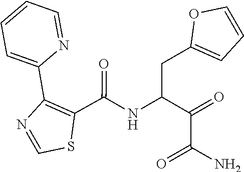 [SMILES: NC(=O)C(=O)C(Cc1ccco1)NC(=O)c1scnc1-c1ccccn1]

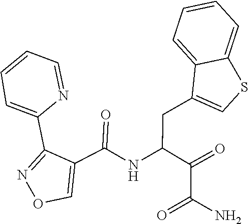 [SMILES: NC(=O)C(=O)C(Cc1csc2ccccc12)NC(=O)c1conc1-c1ccccn1]

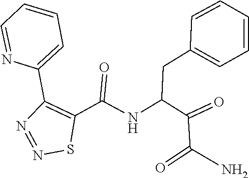 [SMILES: NC(=O)C(=O)C(Cc1ccccc1)NC(=O)c1snnc1-c1ccccn1]

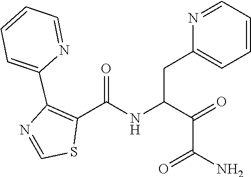 [SMILES: NC(=O)C(=O)C(Cc1ccccn1)NC(=O)c1scnc1-c1ccccn1]

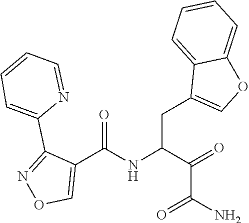 [SMILES: NC(=O)C(=O)C(Cc1coc2ccccc12)NC(=O)c1conc1-c1ccccn1]

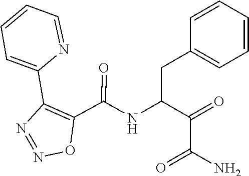 [SMILES: NC(=O)C(=O)C(Cc1ccccc1)NC(=O)c1onnc1-c1ccccn1]

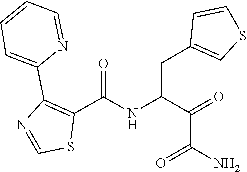 [SMILES: NC(=O)C(=O)C(Cc1ccsc1)NC(=O)c1scnc1-c1ccccn1]

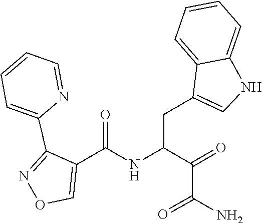 [SMILES: NC(=O)C(=O)C(Cc1c[nH]c2ccccc12)NC(=O)c1conc1-c1ccccn1]

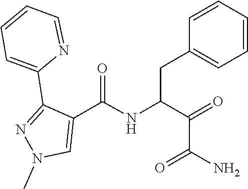 [SMILES: Cn1cc(C(=O)NC(Cc2ccccc2)C(=O)C(N)=O)c(-c2ccccn2)n1]

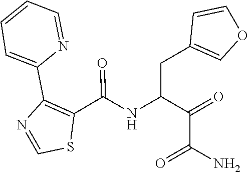 [SMILES: NC(=O)C(=O)C(Cc1ccoc1)NC(=O)c1scnc1-c1ccccn1]

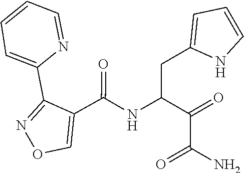 [SMILES: NC(=O)C(=O)C(Cc1ccc[nH]1)NC(=O)c1conc1-c1ccccn1]

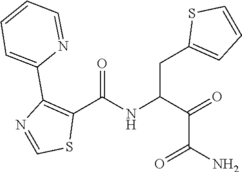 [SMILES: NC(=O)C(=O)C(Cc1cccs1)NC(=O)c1scnc1-c1ccccn1]

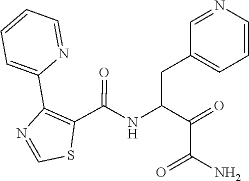 [SMILES: NC(=O)C(=O)C(Cc1cccnc1)NC(=O)c1scnc1-c1ccccn1]

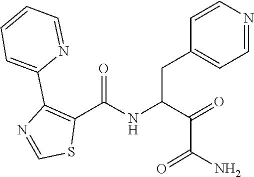 [SMILES: NC(=O)C(=O)C(Cc1ccncc1)NC(=O)c1scnc1-c1ccccn1]

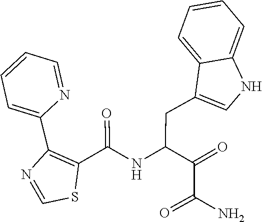 [SMILES: NC(=O)C(=O)C(Cc1c[nH]c2ccccc12)NC(=O)c1scnc1-c1ccccn1]

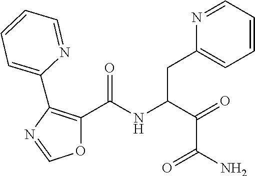 [SMILES: NC(=O)C(=O)C(Cc1ccccn1)NC(=O)c1ocnc1-c1ccccn1]

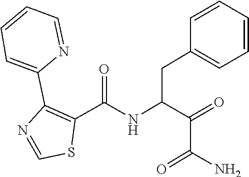 [SMILES: NC(=O)C(=O)C(Cc1ccccc1)NC(=O)c1scnc1-c1ccccn1]

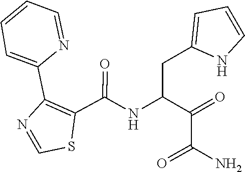 [SMILES: NC(=O)C(=O)C(Cc1ccc[nH]1)NC(=O)c1scnc1-c1ccccn1]

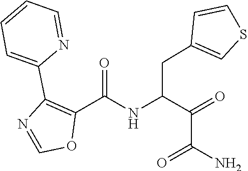 [SMILES: NC(=O)C(=O)C(Cc1ccsc1)NC(=O)c1ocnc1-c1ccccn1]

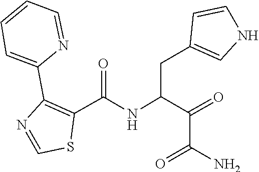 [SMILES: NC(=O)C(=O)C(Cc1cc[nH]c1)NC(=O)c1scnc1-c1ccccn1]

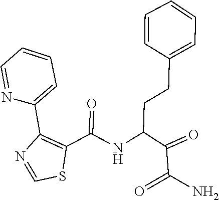 [SMILES: NC(=O)C(=O)C(CCc1ccccc1)NC(=O)c1scnc1-c1ccccn1]

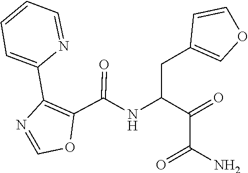 [SMILES: NC(=O)C(=O)C(Cc1ccoc1)NC(=O)c1ocnc1-c1ccccn1]

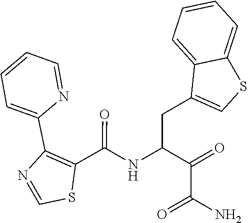 [SMILES: NC(=O)C(=O)C(Cc1csc2ccccc12)NC(=O)c1scnc1-c1ccccn1]

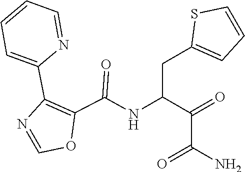 [SMILES: NC(=O)C(=O)C(Cc1cccs1)NC(=O)c1ocnc1-c1ccccn1]

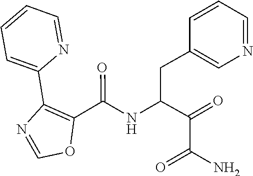 [SMILES: NC(=O)C(=O)C(Cc1cccnc1)NC(=O)c1ocnc1-c1ccccn1]

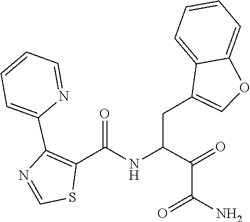 [SMILES: NC(=O)C(=O)C(Cc1coc2ccccc12)NC(=O)c1scnc1-c1ccccn1]

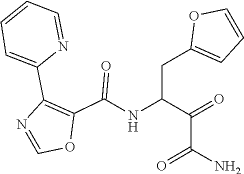 [SMILES: NC(=O)C(=O)C(Cc1ccco1)NC(=O)c1ocnc1-c1ccccn1]

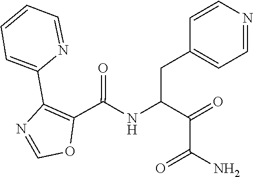 [SMILES: NC(=O)C(=O)C(Cc1ccncc1)NC(=O)c1ocnc1-c1ccccn1]

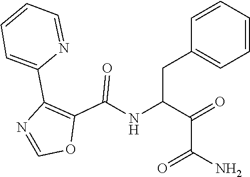 [SMILES: NC(=O)C(=O)C(Cc1ccccc1)NC(=O)c1ocnc1-c1ccccn1]

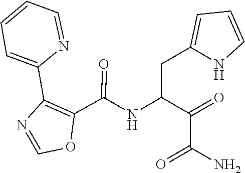 [SMILES: NC(=O)C(=O)C(Cc1ccc[nH]1)NC(=O)c1ocnc1-c1ccccn1]

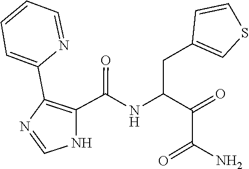 [SMILES: NC(=O)C(=O)C(Cc1ccsc1)NC(=O)c1[nH]cnc1-c1ccccn1]

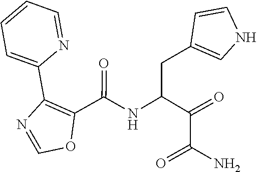 [SMILES: NC(=O)C(=O)C(Cc1cc[nH]c1)NC(=O)c1ocnc1-c1ccccn1]

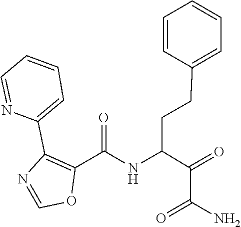 [SMILES: NC(=O)C(=O)C(CCc1ccccc1)NC(=O)c1ocnc1-c1ccccn1]

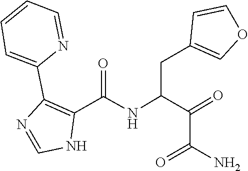 [SMILES: NC(=O)C(=O)C(Cc1ccoc1)NC(=O)c1[nH]cnc1-c1ccccn1]

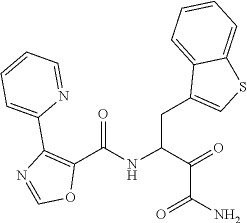 [SMILES: NC(=O)C(=O)C(Cc1csc2ccccc12)NC(=O)c1ocnc1-c1ccccn1]

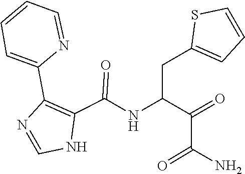 [SMILES: NC(=O)C(=O)C(Cc1cccs1)NC(=O)c1[nH]cnc1-c1ccccn1]

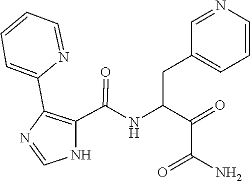 [SMILES: NC(=O)C(=O)C(Cc1cccnc1)NC(=O)c1[nH]cnc1-c1ccccn1]

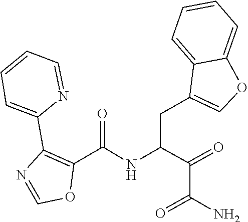 [SMILES: NC(=O)C(=O)C(Cc1coc2ccccc12)NC(=O)c1ocnc1-c1ccccn1]

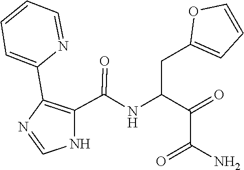 [SMILES: NC(=O)C(=O)C(Cc1ccco1)NC(=O)c1[nH]cnc1-c1ccccn1]

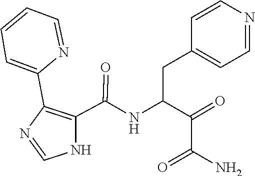 [SMILES: NC(=O)C(=O)C(Cc1ccncc1)NC(=O)c1[nH]cnc1-c1ccccn1]

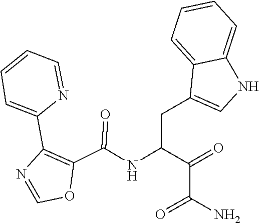 [SMILES: NC(=O)C(=O)C(Cc1c[nH]c2ccccc12)NC(=O)c1ocnc1-c1ccccn1]

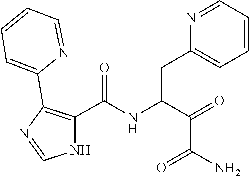 [SMILES: NC(=O)C(=O)C(Cc1ccccn1)NC(=O)c1[nH]cnc1-c1ccccn1]

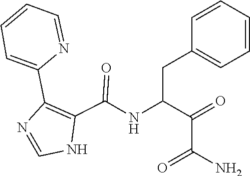 [SMILES: NC(=O)C(=O)C(Cc1ccccc1)NC(=O)c1[nH]cnc1-c1ccccn1]

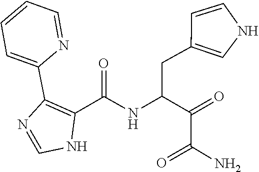 [SMILES: NC(=O)C(=O)C(Cc1cc[nH]c1)NC(=O)c1[nH]cnc1-c1ccccn1]

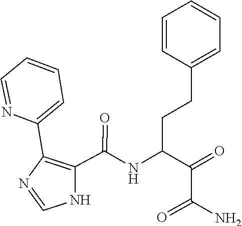 [SMILES: NC(=O)C(=O)C(CCc1ccccc1)NC(=O)c1[nH]cnc1-c1ccccn1]

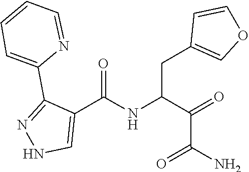 [SMILES: NC(=O)C(=O)C(Cc1ccoc1)NC(=O)c1c[nH]nc1-c1ccccn1]

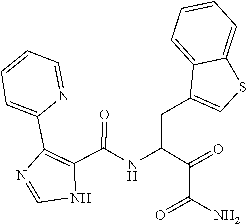 [SMILES: NC(=O)C(=O)C(Cc1csc2ccccc12)NC(=O)c1[nH]cnc1-c1ccccn1]

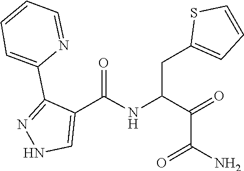 [SMILES: NC(=O)C(=O)C(Cc1cccs1)NC(=O)c1c[nH]nc1-c1ccccn1]

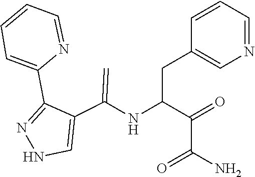 [SMILES: C=C(NC(Cc1cccnc1)C(=O)C(N)=O)c1c[nH]nc1-c1ccccn1]

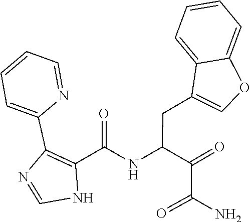 [SMILES: NC(=O)C(=O)C(Cc1coc2ccccc12)NC(=O)c1[nH]cnc1-c1ccccn1]

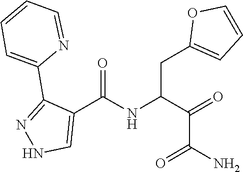 [SMILES: NC(=O)C(=O)C(Cc1ccco1)NC(=O)c1c[nH]nc1-c1ccccn1]

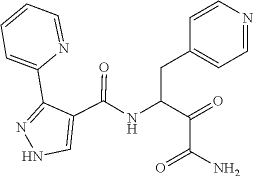 [SMILES: NC(=O)C(=O)C(Cc1ccncc1)NC(=O)c1c[nH]nc1-c1ccccn1]

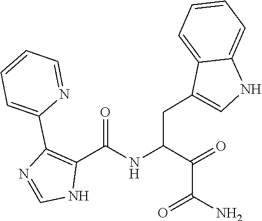 [SMILES: NC(=O)C(=O)C(Cc1c[nH]c2ccccc12)NC(=O)c1[nH]cnc1-c1ccccn1]

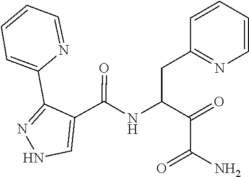 [SMILES: NC(=O)C(=O)C(Cc1ccccn1)NC(=O)c1c[nH]nc1-c1ccccn1]

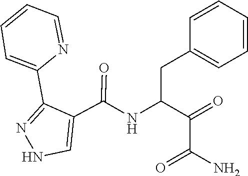 [SMILES: NC(=O)C(=O)C(Cc1ccccc1)NC(=O)c1c[nH]nc1-c1ccccn1]

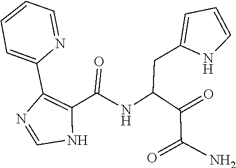 [SMILES: NC(=O)C(=O)C(Cc1ccc[nH]1)NC(=O)c1[nH]cnc1-c1ccccn1]

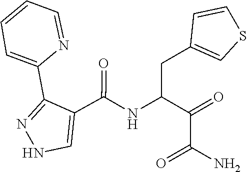 [SMILES: NC(=O)C(=O)C(Cc1ccsc1)NC(=O)c1c[nH]nc1-c1ccccn1]

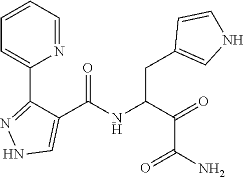 [SMILES: NC(=O)C(=O)C(Cc1cc[nH]c1)NC(=O)c1c[nH]nc1-c1ccccn1]

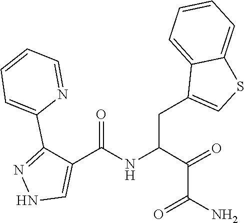 [SMILES: NC(=O)C(=O)C(Cc1csc2ccccc12)NC(=O)c1c[nH]nc1-c1ccccn1]

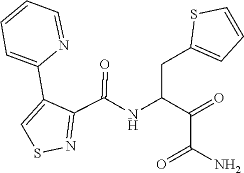 [SMILES: NC(=O)C(=O)C(Cc1cccs1)NC(=O)c1nscc1-c1ccccn1]

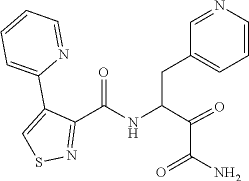 [SMILES: NC(=O)C(=O)C(Cc1cccnc1)NC(=O)c1nscc1-c1ccccn1]

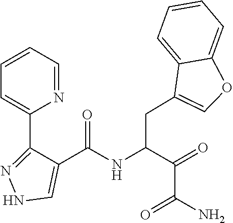

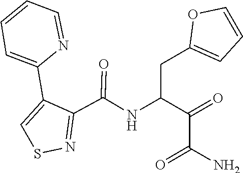 [SMILES: NC(=O)C(=O)C(Cc1ccco1)NC(=O)c1nscc1-c1ccccn1]

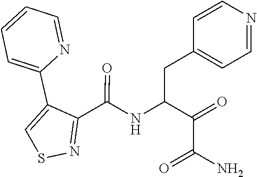 [SMILES: NC(=O)C(=O)C(Cc1ccncc1)NC(=O)c1nscc1-c1ccccn1]

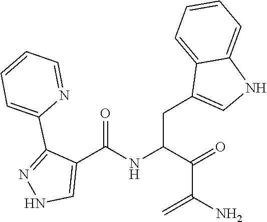 [SMILES: C=C(N)C(=O)C(Cc1c[nH]c2ccccc12)NC(=O)c1c[nH]nc1-c1ccccn1]

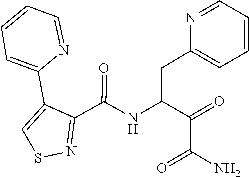 [SMILES: NC(=O)C(=O)C(Cc1ccccn1)NC(=O)c1nscc1-c1ccccn1]

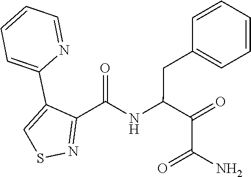 [SMILES: NC(=O)C(=O)C(Cc1ccccc1)NC(=O)c1nscc1-c1ccccn1]

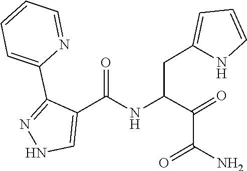 [SMILES: NC(=O)C(=O)C(Cc1ccc[nH]1)NC(=O)c1c[nH]nc1-c1ccccn1]

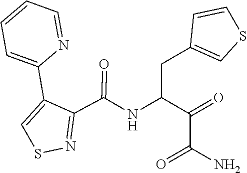 [SMILES: NC(=O)C(=O)C(Cc1ccsc1)NC(=O)c1nscc1-c1ccccn1]

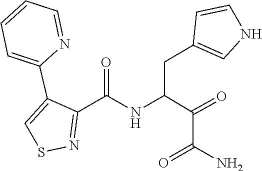 [SMILES: NC(=O)C(=O)C(Cc1cc[nH]c1)NC(=O)c1nscc1-c1ccccn1]

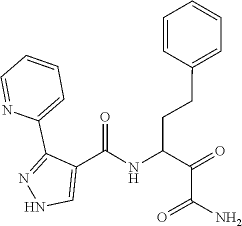 [SMILES: NC(=O)C(=O)C(CCc1ccccc1)NC(=O)c1c[nH]nc1-c1ccccn1]

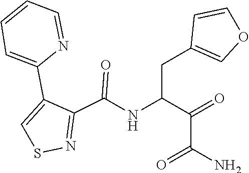 [SMILES: NC(=O)C(=O)C(Cc1ccoc1)NC(=O)c1nscc1-c1ccccn1]

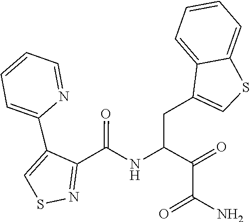 [SMILES: NC(=O)C(=O)C(Cc1csc2ccccc12)NC(=O)c1nscc1-c1ccccn1]

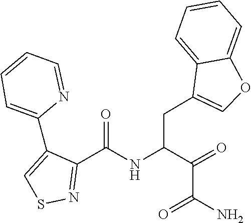 [SMILES: NC(=O)C(=O)C(Cc1coc2ccccc12)NC(=O)c1nscc1-c1ccccn1]

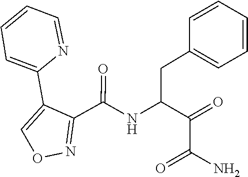 [SMILES: NC(=O)C(=O)C(Cc1ccccc1)NC(=O)c1nocc1-c1ccccn1]

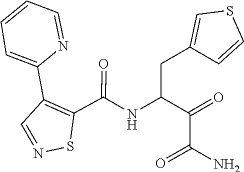 [SMILES: NC(=O)C(=O)C(Cc1ccsc1)NC(=O)c1sncc1-c1ccccn1]

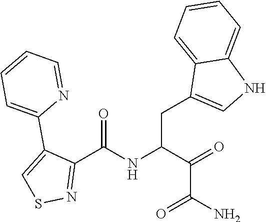 [SMILES: NC(=O)C(=O)C(Cc1c[nH]c2ccccc12)NC(=O)c1nscc1-c1ccccn1]

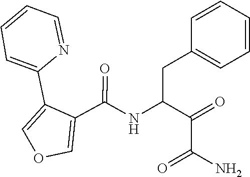 [SMILES: NC(=O)C(=O)C(Cc1ccccc1)NC(=O)c1cocc1-c1ccccn1]

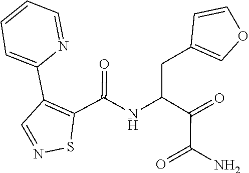 [SMILES: NC(=O)C(=O)C(Cc1ccoc1)NC(=O)c1sncc1-c1ccccn1]

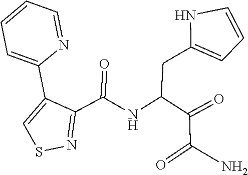 [SMILES: NC(=O)C(=O)C(Cc1ccc[nH]1)NC(=O)c1nscc1-c1ccccn1]

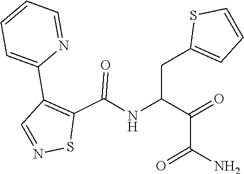 [SMILES: NC(=O)C(=O)C(Cc1cccs1)NC(=O)c1sncc1-c1ccccn1]

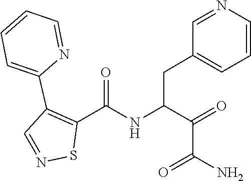 [SMILES: NC(=O)C(=O)C(Cc1cccnc1)NC(=O)c1sncc1-c1ccccn1]

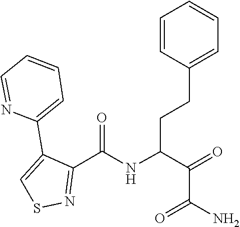 [SMILES: NC(=O)C(=O)C(CCc1ccccc1)NC(=O)c1nscc1-c1ccccn1]

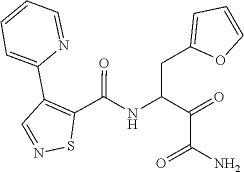 [SMILES: NC(=O)C(=O)C(Cc1ccco1)NC(=O)c1sncc1-c1ccccn1]

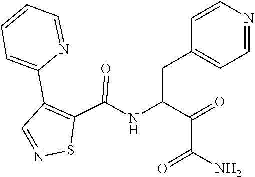 [SMILES: NC(=O)C(=O)C(Cc1ccncc1)NC(=O)c1sncc1-c1ccccn1]

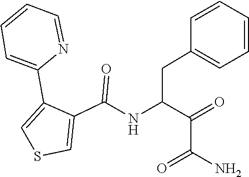 [SMILES: NC(=O)C(=O)C(Cc1ccccc1)NC(=O)c1cscc1-c1ccccn1]

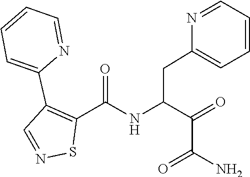 [SMILES: NC(=O)C(=O)C(Cc1ccccn1)NC(=O)c1sncc1-c1ccccn1]

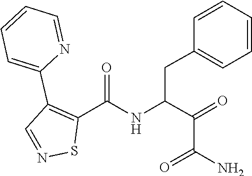 [SMILES: NC(=O)C(=O)C(Cc1ccccc1)NC(=O)c1sncc1-c1ccccn1]

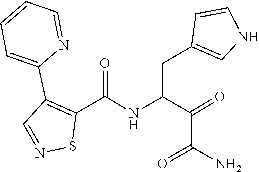 [SMILES: NC(=O)C(=O)C(Cc1cc[nH]c1)NC(=O)c1sncc1-c1ccccn1]

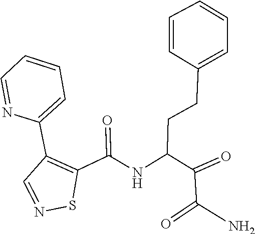 [SMILES: NC(=O)C(=O)C(CCc1ccccc1)NC(=O)c1sncc1-c1ccccn1]

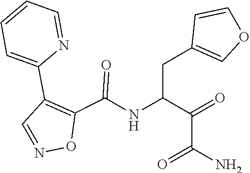 [SMILES: NC(=O)C(=O)C(Cc1ccoc1)NC(=O)c1oncc1-c1ccccn1]

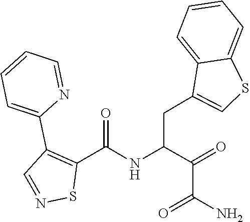 [SMILES: NC(=O)C(=O)C(Cc1csc2ccccc12)NC(=O)c1sncc1-c1ccccn1]

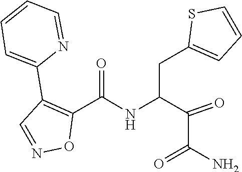 [SMILES: NC(=O)C(=O)C(Cc1cccs1)NC(=O)c1oncc1-c1ccccn1]

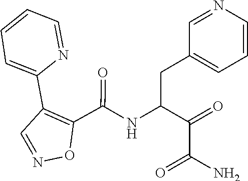 [SMILES: NC(=O)C(=O)C(Cc1cccnc1)NC(=O)c1oncc1-c1ccccn1]

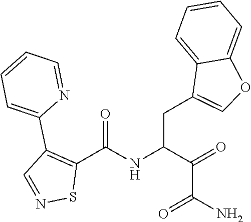 [SMILES: NC(=O)C(=O)C(Cc1coc2ccccc12)NC(=O)c1sncc1-c1ccccn1]

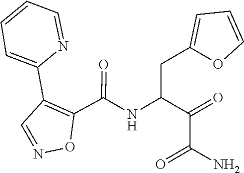 [SMILES: NC(=O)C(=O)C(Cc1ccco1)NC(=O)c1oncc1-c1ccccn1]

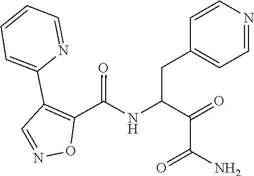 [SMILES: NC(=O)C(=O)C(Cc1ccncc1)NC(=O)c1oncc1-c1ccccn1]

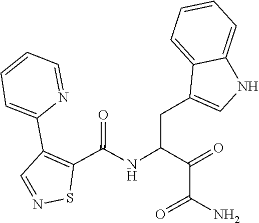 [SMILES: NC(=O)C(=O)C(Cc1c[nH]c2ccccc12)NC(=O)c1sncc1-c1ccccn1]

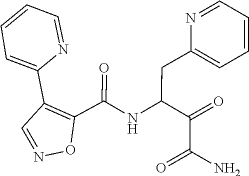 [SMILES: NC(=O)C(=O)C(Cc1ccccn1)NC(=O)c1oncc1-c1ccccn1]

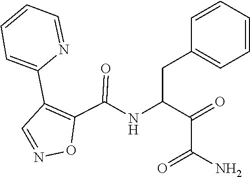 [SMILES: NC(=O)C(=O)C(Cc1ccccc1)NC(=O)c1oncc1-c1ccccn1]

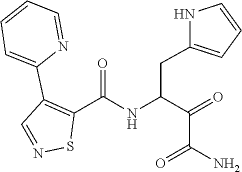 [SMILES: NC(=O)C(=O)C(Cc1ccc[nH]1)NC(=O)c1sncc1-c1ccccn1]

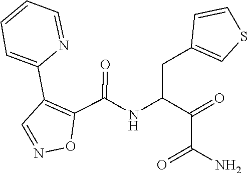 [SMILES: NC(=O)C(=O)C(Cc1ccsc1)NC(=O)c1oncc1-c1ccccn1]

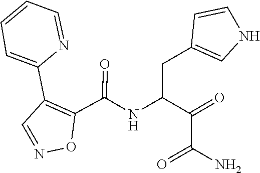 [SMILES: NC(=O)C(=O)C(Cc1cc[nH]c1)NC(=O)c1oncc1-c1ccccn1]

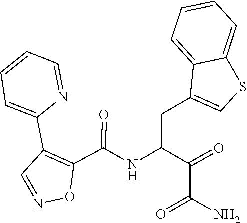 [SMILES: NC(=O)C(=O)C(Cc1csc2ccccc12)NC(=O)c1oncc1-c1ccccn1]

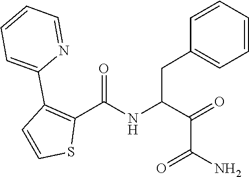 [SMILES: NC(=O)C(=O)C(Cc1ccccc1)NC(=O)c1sccc1-c1ccccn1]

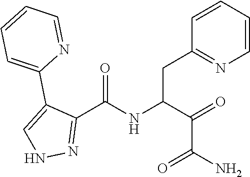 [SMILES: NC(=O)C(=O)C(Cc1ccccn1)NC(=O)c1n[nH]cc1-c1ccccn1]

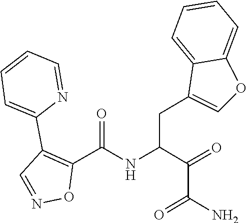 [SMILES: NC(=O)C(=O)C(Cc1coc2ccccc12)NC(=O)c1oncc1-c1ccccn1]

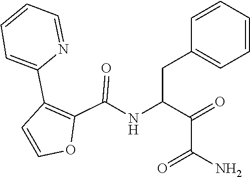 [SMILES: NC(=O)C(=O)C(Cc1ccccc1)NC(=O)c1occc1-c1ccccn1]

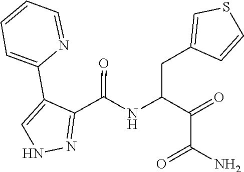 [SMILES: NC(=O)C(=O)C(Cc1ccsc1)NC(=O)c1n[nH]cc1-c1ccccn1]

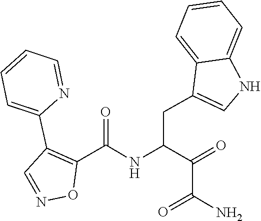 [SMILES: NC(=O)C(=O)C(Cc1c[nH]c2ccccc12)NC(=O)c1oncc1-c1ccccn1]

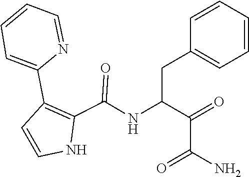 [SMILES: NC(=O)C(=O)C(Cc1ccccc1)NC(=O)c1[nH]ccc1-c1ccccn1]

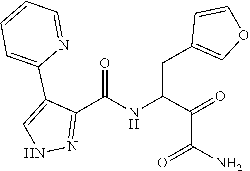 [SMILES: NC(=O)C(=O)C(Cc1ccoc1)NC(=O)c1n[nH]cc1-c1ccccn1]

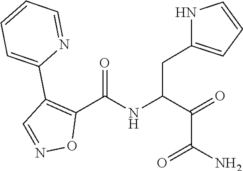 [SMILES: NC(=O)C(=O)C(Cc1ccc[nH]1)NC(=O)c1oncc1-c1ccccn1]

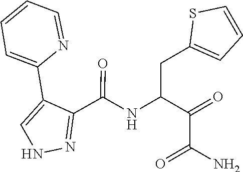 [SMILES: NC(=O)C(=O)C(Cc1cccs1)NC(=O)c1n[nH]cc1-c1ccccn1]

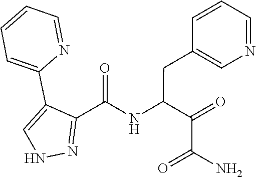 [SMILES: NC(=O)C(=O)C(Cc1cccnc1)NC(=O)c1n[nH]cc1-c1ccccn1]

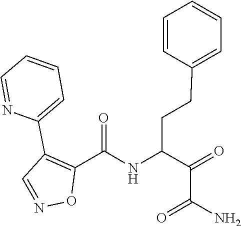 [SMILES: NC(=O)C(=O)C(CCc1ccccc1)NC(=O)c1oncc1-c1ccccn1]

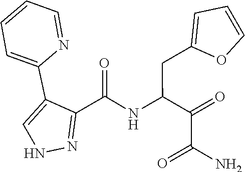 [SMILES: NC(=O)C(=O)C(Cc1ccco1)NC(=O)c1n[nH]cc1-c1ccccn1]

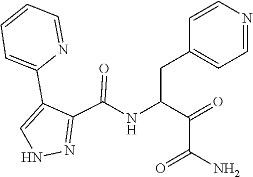 [SMILES: NC(=O)C(=O)C(Cc1ccncc1)NC(=O)c1n[nH]cc1-c1ccccn1]

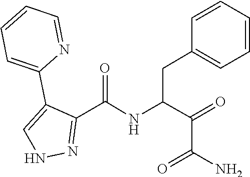 [SMILES: NC(=O)C(=O)C(Cc1ccccc1)NC(=O)c1n[nH]cc1-c1ccccn1]

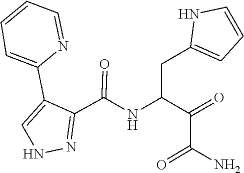 [SMILES: NC(=O)C(=O)C(Cc1ccc[nH]1)NC(=O)c1n[nH]cc1-c1ccccn1]

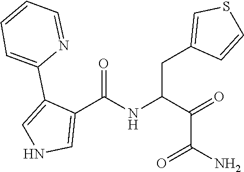 [SMILES: NC(=O)C(=O)C(Cc1ccsc1)NC(=O)c1c[nH]cc1-c1ccccn1]

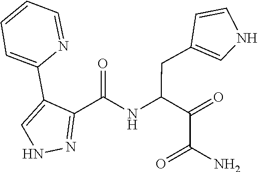 [SMILES: NC(=O)C(=O)C(Cc1cc[nH]c1)NC(=O)c1n[nH]cc1-c1ccccn1]

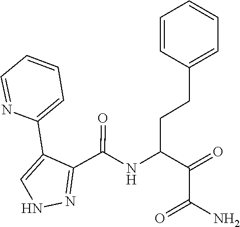 [SMILES: NC(=O)C(=O)C(CCc1ccccc1)NC(=O)c1n[nH]cc1-c1ccccn1]

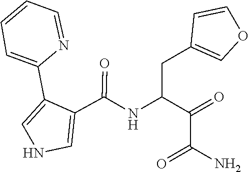 [SMILES: NC(=O)C(=O)C(Cc1ccoc1)NC(=O)c1c[nH]cc1-c1ccccn1]

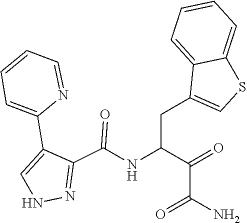 [SMILES: NC(=O)C(=O)C(Cc1csc2ccccc12)NC(=O)c1n[nH]cc1-c1ccccn1]

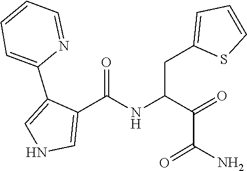 [SMILES: NC(=O)C(=O)C(Cc1cccs1)NC(=O)c1c[nH]cc1-c1ccccn1]

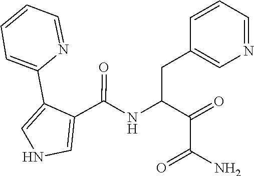 [SMILES: NC(=O)C(=O)C(Cc1cccnc1)NC(=O)c1c[nH]cc1-c1ccccn1]

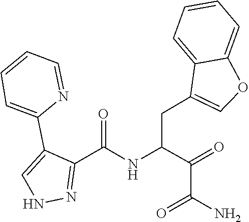 [SMILES: NC(=O)C(=O)C(Cc1coc2ccccc12)NC(=O)c1n[nH]cc1-c1ccccn1]

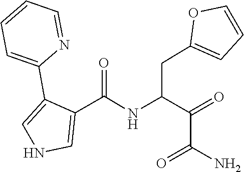 [SMILES: NC(=O)C(=O)C(Cc1ccco1)NC(=O)c1c[nH]cc1-c1ccccn1]

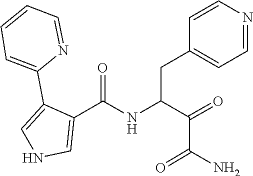 [SMILES: NC(=O)C(=O)C(Cc1ccncc1)NC(=O)c1c[nH]cc1-c1ccccn1]

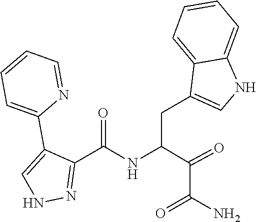 [SMILES: NC(=O)C(=O)C(Cc1c[nH]c2ccccc12)NC(=O)c1n[nH]cc1-c1ccccn1]

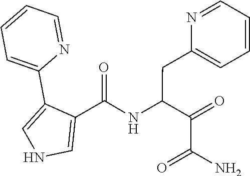 [SMILES: NC(=O)C(=O)C(Cc1ccccn1)NC(=O)c1c[nH]cc1-c1ccccn1]

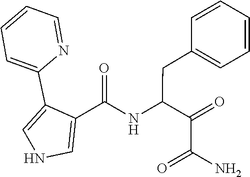 [SMILES: NC(=O)C(=O)C(Cc1ccccc1)NC(=O)c1c[nH]cc1-c1ccccn1]

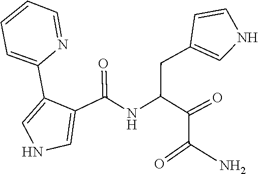 [SMILES: NC(=O)C(=O)C(Cc1cc[nH]c1)NC(=O)c1c[nH]cc1-c1ccccn1]

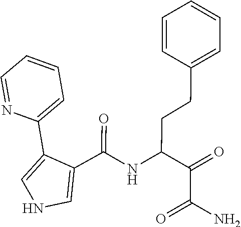 [SMILES: NC(=O)C(=O)C(CCc1ccccc1)NC(=O)c1c[nH]cc1-c1ccccn1]

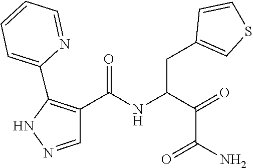 [SMILES: NC(=O)C(=O)C(Cc1ccsc1)NC(=O)c1cn[nH]c1-c1ccccn1]

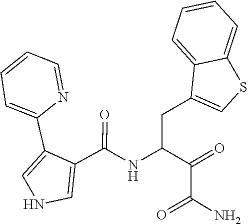 [SMILES: NC(=O)C(=O)C(Cc1csc2ccccc12)NC(=O)c1c[nH]cc1-c1ccccn1]

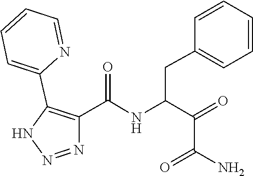 [SMILES: NC(=O)C(=O)C(Cc1ccccc1)NC(=O)c1nn[nH]c1-c1ccccn1]

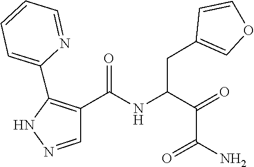 [SMILES: NC(=O)C(=O)C(Cc1ccoc1)NC(=O)c1cn[nH]c1-c1ccccn1]

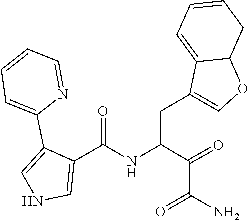 [SMILES: NC(=O)C(=O)C(CC1=COC2CC=CC=C12)NC(=O)c1c[nH]cc1-c1ccccn1]

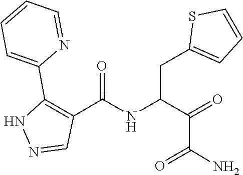 [SMILES: NC(=O)C(=O)C(Cc1cccs1)NC(=O)c1cn[nH]c1-c1ccccn1]

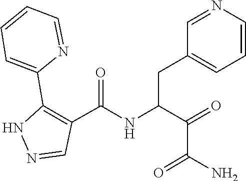 [SMILES: NC(=O)C(=O)C(Cc1cccnc1)NC(=O)c1cn[nH]c1-c1ccccn1]

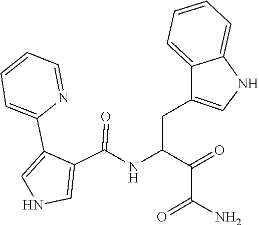 [SMILES: NC(=O)C(=O)C(Cc1c[nH]c2ccccc12)NC(=O)c1c[nH]cc1-c1ccccn1]

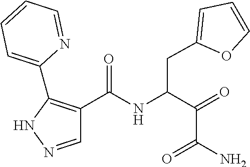 [SMILES: NC(=O)C(=O)C(Cc1ccco1)NC(=O)c1cn[nH]c1-c1ccccn1]

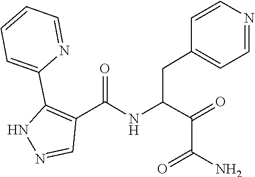 [SMILES: NC(=O)C(=O)C(Cc1ccncc1)NC(=O)c1cn[nH]c1-c1ccccn1]

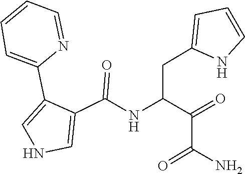 [SMILES: NC(=O)C(=O)C(Cc1ccc[nH]1)NC(=O)c1c[nH]cc1-c1ccccn1]

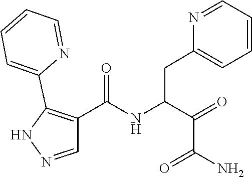 [SMILES: NC(=O)C(=O)C(Cc1ccccn1)NC(=O)c1cn[nH]c1-c1ccccn1]

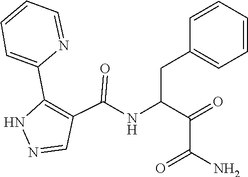 [SMILES: NC(=O)C(=O)C(Cc1ccccc1)NC(=O)c1cn[nH]c1-c1ccccn1]

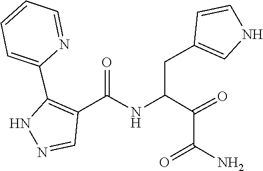 [SMILES: NC(=O)C(=O)C(Cc1cc[nH]c1)NC(=O)c1cn[nH]c1-c1ccccn1]

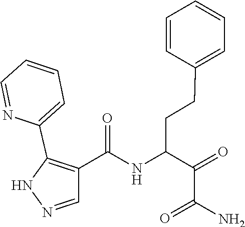 [SMILES: NC(=O)C(=O)C(CCc1ccccc1)NC(=O)c1cn[nH]c1-c1ccccn1]

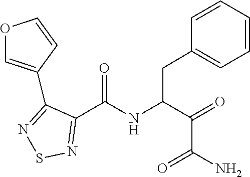 [SMILES: NC(=O)C(=O)C(Cc1ccccc1)NC(=O)c1nsnc1-c1ccoc1]

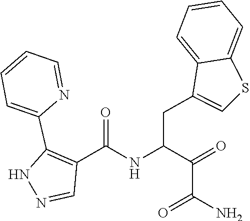 [SMILES: NC(=O)C(=O)C(Cc1csc2ccccc12)NC(=O)c1cn[nH]c1-c1ccccn1]

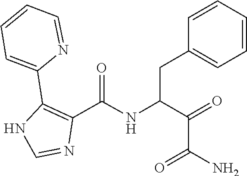 [SMILES: NC(=O)C(=O)C(Cc1ccccc1)NC(=O)c1nc[nH]c1-c1ccccn1]

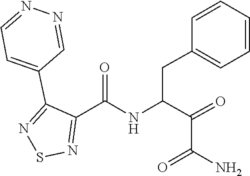 [SMILES: NC(=O)C(=O)C(Cc1ccccc1)NC(=O)c1nsnc1-c1ccnnc1]

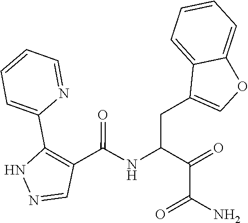 [SMILES: NC(=O)C(=O)C(Cc1coc2ccccc12)NC(=O)c1cn[nH]c1-c1ccccn1]

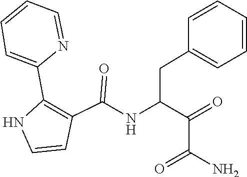 [SMILES: NC(=O)C(=O)C(Cc1ccccc1)NC(=O)c1cc[nH]c1-c1ccccn1]

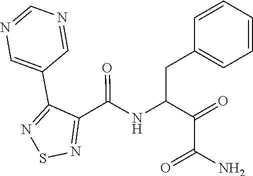 [SMILES: NC(=O)C(=O)C(Cc1ccccc1)NC(=O)c1nsnc1-c1cncnc1]

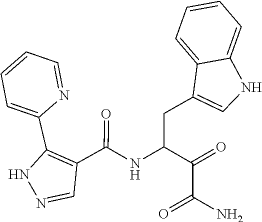 [SMILES: NC(=O)C(=O)C(Cc1c[nH]c2ccccc12)NC(=O)c1cn[nH]c1-c1ccccn1]

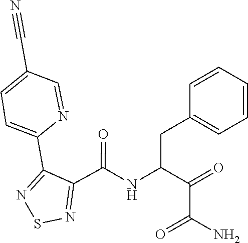 [SMILES: N#Cc1ccc(-c2nsnc2C(=O)NC(Cc2ccccc2)C(=O)C(N)=O)nc1]

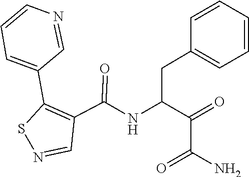 [SMILES: NC(=O)C(=O)C(Cc1ccccc1)NC(=O)c1cnsc1-c1cccnc1]

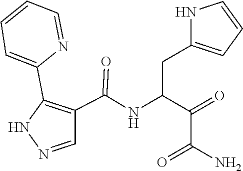 [SMILES: NC(=O)C(=O)C(Cc1ccc[nH]1)NC(=O)c1cn[nH]c1-c1ccccn1]

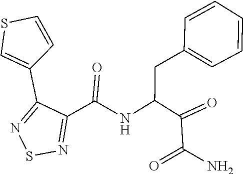 [SMILES: NC(=O)C(=O)C(Cc1ccccc1)NC(=O)c1nsnc1-c1ccsc1]

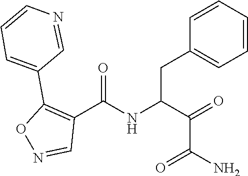 [SMILES: NC(=O)C(=O)C(Cc1ccccc1)NC(=O)c1cnoc1-c1cccnc1]

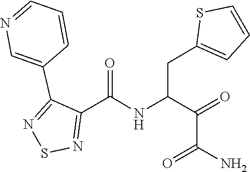 [SMILES: NC(=O)C(=O)C(Cc1cccs1)NC(=O)c1nsnc1-c1cccnc1]

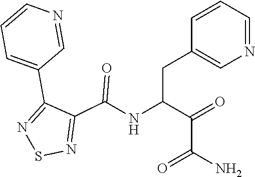 [SMILES: NC(=O)C(=O)C(Cc1cccnc1)NC(=O)c1nsnc1-c1cccnc1]

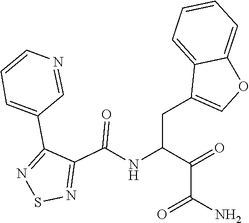 [SMILES: NC(=O)C(=O)C(Cc1coc2ccccc12)NC(=O)c1nsnc1-c1cccnc1]

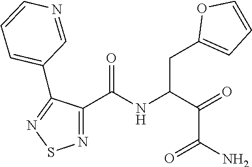 [SMILES: NC(=O)C(=O)C(Cc1ccco1)NC(=O)c1nsnc1-c1cccnc1]

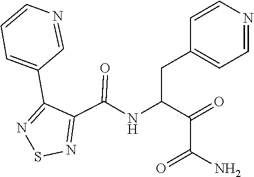 [SMILES: NC(=O)C(=O)C(Cc1ccncc1)NC(=O)c1nsnc1-c1cccnc1]

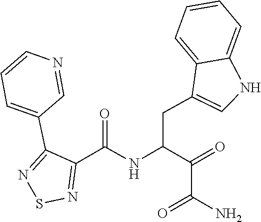 [SMILES: NC(=O)C(=O)C(Cc1c[nH]c2ccccc12)NC(=O)c1nsnc1-c1cccnc1]

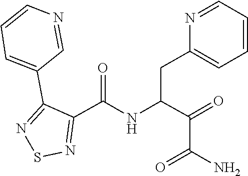 [SMILES: NC(=O)C(=O)C(Cc1ccccn1)NC(=O)c1nsnc1-c1cccnc1]

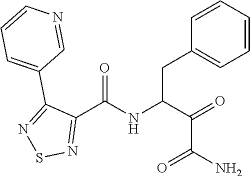 [SMILES: NC(=O)C(=O)C(Cc1ccccc1)NC(=O)c1nsnc1-c1cccnc1]

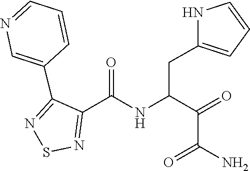 [SMILES: NC(=O)C(=O)C(Cc1ccc[nH]1)NC(=O)c1nsnc1-c1cccnc1]

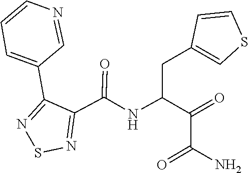 [SMILES: NC(=O)C(=O)C(Cc1ccsc1)NC(=O)c1nsnc1-c1cccnc1]

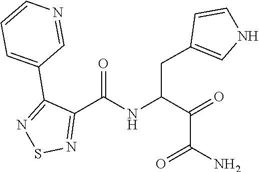 [SMILES: NC(=O)C(=O)C(Cc1cc[nH]c1)NC(=O)c1nsnc1-c1cccnc1]

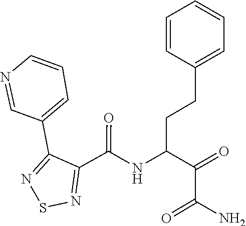 [SMILES: NC(=O)C(=O)C(CCc1ccccc1)NC(=O)c1nsnc1-c1cccnc1]

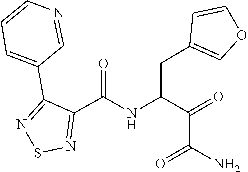 [SMILES: NC(=O)C(=O)C(Cc1ccoc1)NC(=O)c1nsnc1-c1cccnc1]

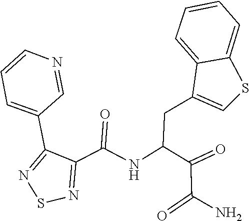 [SMILES: NC(=O)C(=O)C(Cc1csc2ccccc12)NC(=O)c1nsnc1-c1cccnc1]

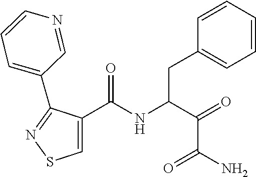 [SMILES: NC(=O)C(=O)C(Cc1ccccc1)NC(=O)c1csnc1-c1cccnc1]

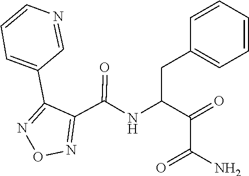 [SMILES: NC(=O)C(=O)C(Cc1ccccc1)NC(=O)c1nonc1-c1cccnc1]

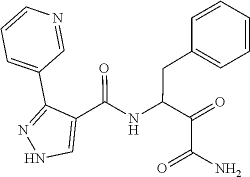 [SMILES: NC(=O)C(=O)C(Cc1ccccc1)NC(=O)c1c[nH]nc1-c1cccnc1]

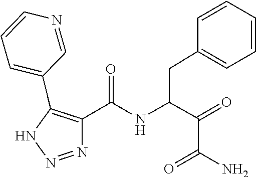 [SMILES: NC(=O)C(=O)C(Cc1ccccc1)NC(=O)c1nn[nH]c1-c1cccnc1]

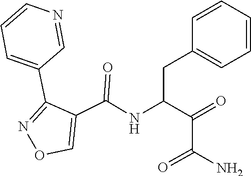 [SMILES: NC(=O)C(=O)C(Cc1ccccc1)NC(=O)c1conc1-c1cccnc1]

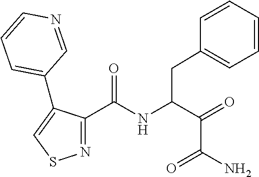 [SMILES: NC(=O)C(=O)C(Cc1ccccc1)NC(=O)c1nscc1-c1cccnc1]

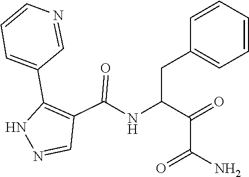 [SMILES: NC(=O)C(=O)C(Cc1ccccc1)NC(=O)c1cn[nH]c1-c1cccnc1]

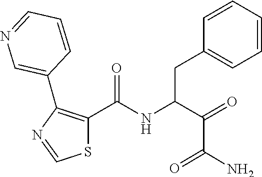 [SMILES: NC(=O)C(=O)C(Cc1ccccc1)NC(=O)c1scnc1-c1cccnc1]

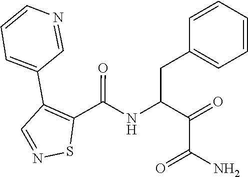 [SMILES: NC(=O)C(=O)C(Cc1ccccc1)NC(=O)c1sncc1-c1cccnc1]

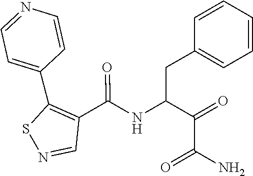 [SMILES: NC(=O)C(=O)C(Cc1ccccc1)NC(=O)c1cnsc1-c1ccncc1]

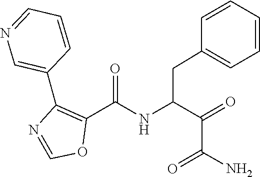 [SMILES: NC(=O)C(=O)C(Cc1ccccc1)NC(=O)c1ocnc1-c1cccnc1]

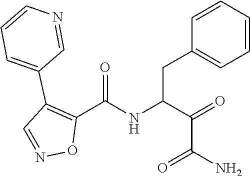 [SMILES: NC(=O)C(=O)C(Cc1ccccc1)NC(=O)c1oncc1-c1cccnc1]

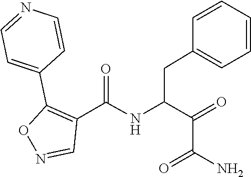 [SMILES: NC(=O)C(=O)C(Cc1ccccc1)NC(=O)c1cnoc1-c1ccncc1]

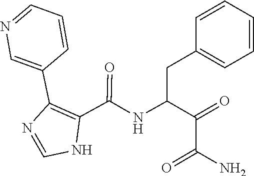 [SMILES: NC(=O)C(=O)C(Cc1ccccc1)NC(=O)c1[nH]cnc1-c1cccnc1]

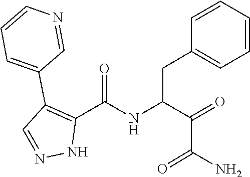 [SMILES: NC(=O)C(=O)C(Cc1ccccc1)NC(=O)c1[nH]ncc1-c1cccnc1]

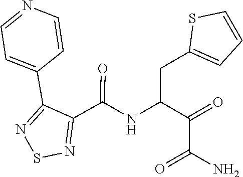 [SMILES: NC(=O)C(=O)C(Cc1cccs1)NC(=O)c1nsnc1-c1ccncc1]

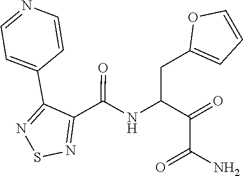 [SMILES: NC(=O)C(=O)C(Cc1ccco1)NC(=O)c1nsnc1-c1ccncc1]

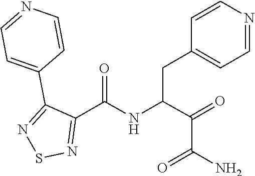 [SMILES: NC(=O)C(=O)C(Cc1ccncc1)NC(=O)c1nsnc1-c1ccncc1]

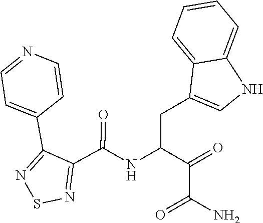 [SMILES: NC(=O)C(=O)C(Cc1c[nH]c2ccccc12)NC(=O)c1nsnc1-c1ccncc1]

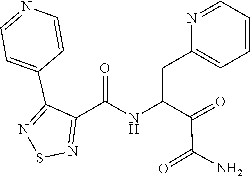 [SMILES: NC(=O)C(=O)C(Cc1ccccn1)NC(=O)c1nsnc1-c1ccncc1]

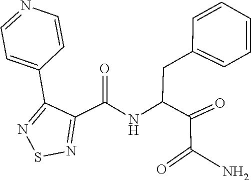 [SMILES: NC(=O)C(=O)C(Cc1ccccc1)NC(=O)c1nsnc1-c1ccncc1]

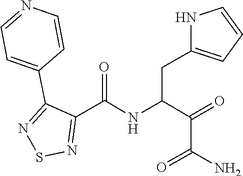 [SMILES: NC(=O)C(=O)C(Cc1ccc[nH]1)NC(=O)c1nsnc1-c1ccncc1]

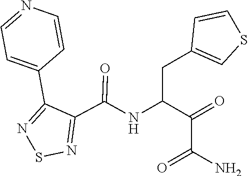 [SMILES: NC(=O)C(=O)C(Cc1ccsc1)NC(=O)c1nsnc1-c1ccncc1]

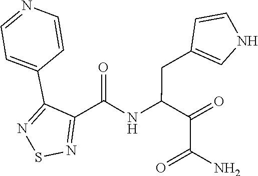 [SMILES: NC(=O)C(=O)C(Cc1cc[nH]c1)NC(=O)c1nsnc1-c1ccncc1]

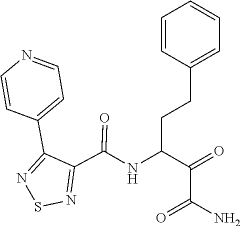 [SMILES: NC(=O)C(=O)C(CCc1ccccc1)NC(=O)c1nsnc1-c1ccncc1]

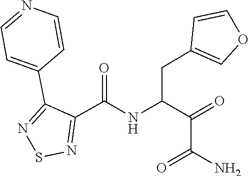 [SMILES: NC(=O)C(=O)C(Cc1ccoc1)NC(=O)c1nsnc1-c1ccncc1]

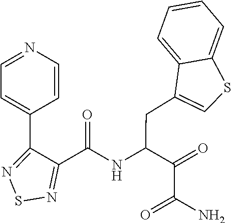

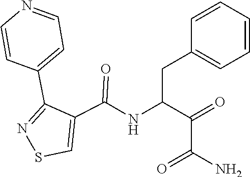 [SMILES: NC(=O)C(=O)C(Cc1ccccc1)NC(=O)c1csnc1-c1ccncc1]

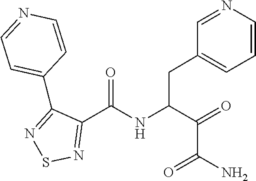 [SMILES: NC(=O)C(=O)C(Cc1cccnc1)NC(=O)c1nsnc1-c1ccncc1]

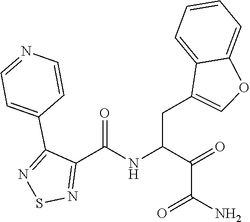 [SMILES: NC(=O)C(=O)C(Cc1coc2ccccc12)NC(=O)c1nsnc1-c1ccncc1]

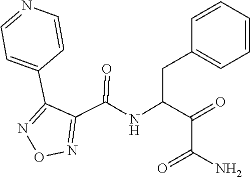 [SMILES: NC(=O)C(=O)C(Cc1ccccc1)NC(=O)c1nonc1-c1ccncc1]

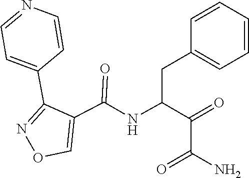 [SMILES: NC(=O)C(=O)C(Cc1ccccc1)NC(=O)c1conc1-c1ccncc1]

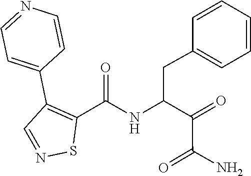 [SMILES: NC(=O)C(=O)C(Cc1ccccc1)NC(=O)c1sncc1-c1ccncc1]

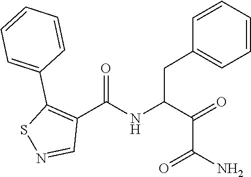 [SMILES: NC(=O)C(=O)C(Cc1ccccc1)NC(=O)c1cnsc1-c1ccccc1]

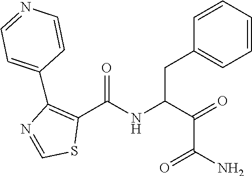 [SMILES: NC(=O)C(=O)C(Cc1ccccc1)NC(=O)c1scnc1-c1ccncc1]

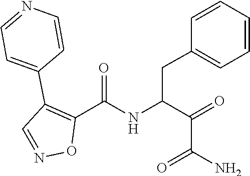 [SMILES: NC(=O)C(=O)C(Cc1ccccc1)NC(=O)c1oncc1-c1ccncc1]

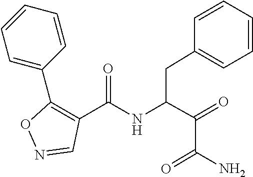 [SMILES: NC(=O)C(=O)C(Cc1ccccc1)NC(=O)c1cnoc1-c1ccccc1]

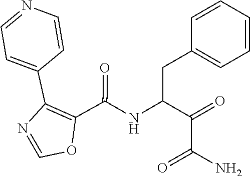 [SMILES: NC(=O)C(=O)C(Cc1ccccc1)NC(=O)c1ocnc1-c1ccncc1]

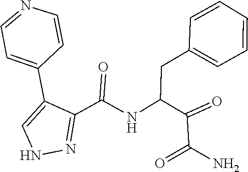 [SMILES: NC(=O)C(=O)C(Cc1ccccc1)NC(=O)c1n[nH]cc1-c1ccncc1]

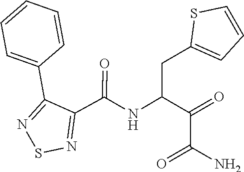 [SMILES: NC(=O)C(=O)C(Cc1cccs1)NC(=O)c1nsnc1-c1ccccc1]

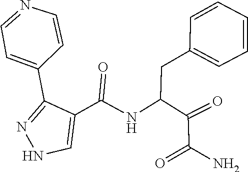 [SMILES: NC(=O)C(=O)C(Cc1ccccc1)NC(=O)c1c[nH]nc1-c1ccncc1]

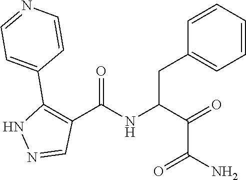 [SMILES: NC(=O)C(=O)C(Cc1ccccc1)NC(=O)c1cn[nH]c1-c1ccncc1]

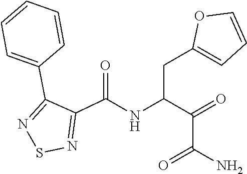 [SMILES: NC(=O)C(=O)C(Cc1ccco1)NC(=O)c1nsnc1-c1ccccc1]

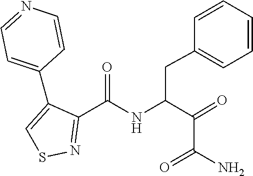 [SMILES: NC(=O)C(=O)C(Cc1ccccc1)NC(=O)c1nscc1-c1ccncc1]

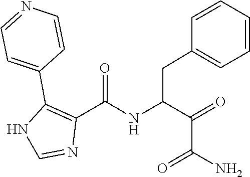 [SMILES: NC(=O)C(=O)C(Cc1ccccc1)NC(=O)c1nc[nH]c1-c1ccncc1]

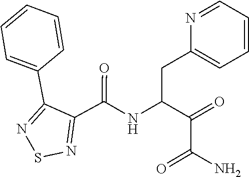 [SMILES: NC(=O)C(=O)C(Cc1ccccn1)NC(=O)c1nsnc1-c1ccccc1]

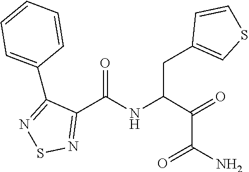 [SMILES: NC(=O)C(=O)C(Cc1ccsc1)NC(=O)c1nsnc1-c1ccccc1]

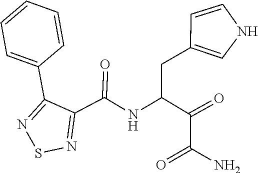 [SMILES: NC(=O)C(=O)C(Cc1cc[nH]c1)NC(=O)c1nsnc1-c1ccccc1]

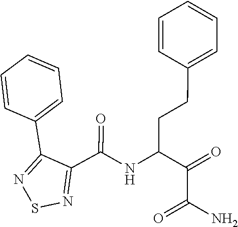 [SMILES: NC(=O)C(=O)C(CCc1ccccc1)NC(=O)c1nsnc1-c1ccccc1]

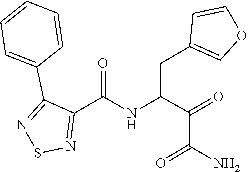 [SMILES: NC(=O)C(=O)C(Cc1ccoc1)NC(=O)c1nsnc1-c1ccccc1]

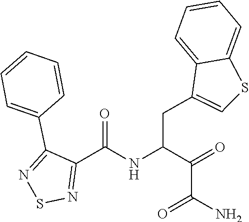 [SMILES: NC(=O)C(=O)C(Cc1csc2ccccc12)NC(=O)c1nsnc1-c1ccccc1]

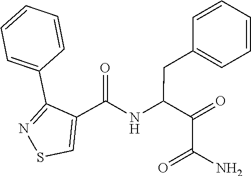 [SMILES: NC(=O)C(=O)C(Cc1ccccc1)NC(=O)c1csnc1-c1ccccc1]

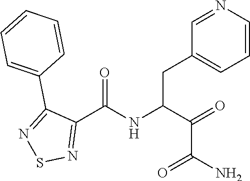 [SMILES: NC(=O)C(=O)C(Cc1cccnc1)NC(=O)c1nsnc1-c1ccccc1]

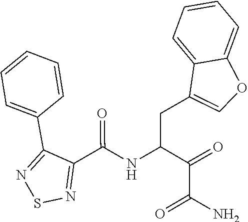 [SMILES: NC(=O)C(=O)C(Cc1coc2ccccc12)NC(=O)c1nsnc1-c1ccccc1]

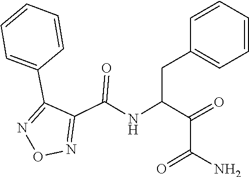 [SMILES: NC(=O)C(=O)C(Cc1ccccc1)NC(=O)c1nonc1-c1ccccc1]

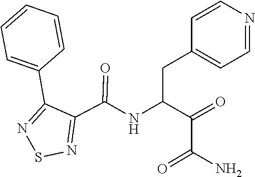 [SMILES: NC(=O)C(=O)C(Cc1ccncc1)NC(=O)c1nsnc1-c1ccccc1]

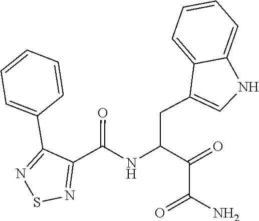 [SMILES: NC(=O)C(=O)C(Cc1c[nH]c2ccccc12)NC(=O)c1nsnc1-c1ccccc1]

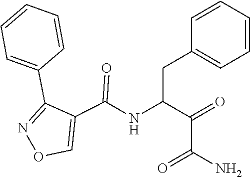 [SMILES: NC(=O)C(=O)C(Cc1ccccc1)NC(=O)c1conc1-c1ccccc1]

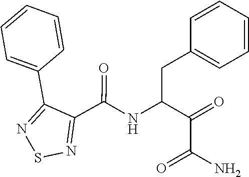 [SMILES: NC(=O)C(=O)C(Cc1ccccc1)NC(=O)c1nsnc1-c1ccccc1]

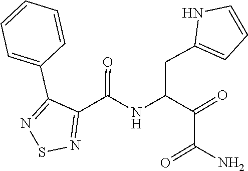 [SMILES: NC(=O)C(=O)C(Cc1ccc[nH]1)NC(=O)c1nsnc1-c1ccccc1]

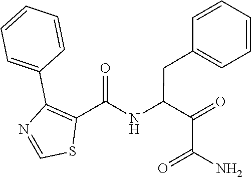 [SMILES: NC(=O)C(=O)C(Cc1ccccc1)NC(=O)c1scnc1-c1ccccc1]

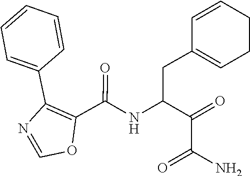 [SMILES: NC(=O)C(=O)C(CC1=CCCC=C1)NC(=O)c1ocnc1-c1ccccc1]

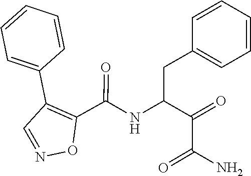 [SMILES: NC(=O)C(=O)C(Cc1ccccc1)NC(=O)c1oncc1-c1ccccc1]

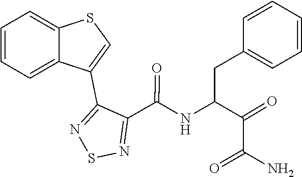 [SMILES: NC(=O)C(=O)C(Cc1ccccc1)NC(=O)c1nsnc1-c1csc2ccccc12]

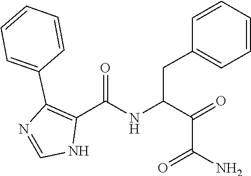 [SMILES: NC(=O)C(=O)C(Cc1ccccc1)NC(=O)c1[nH]cnc1-c1ccccc1]

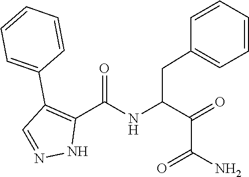 [SMILES: NC(=O)C(=O)C(Cc1ccccc1)NC(=O)c1[nH]ncc1-c1ccccc1]

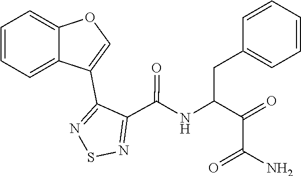 [SMILES: NC(=O)C(=O)C(Cc1ccccc1)NC(=O)c1nsnc1-c1coc2ccccc12]

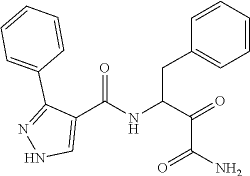 [SMILES: NC(=O)C(=O)C(Cc1ccccc1)NC(=O)c1c[nH]nc1-c1ccccc1]

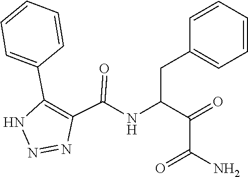 [SMILES: NC(=O)C(=O)C(Cc1ccccc1)NC(=O)c1nn[nH]c1-c1ccccc1]

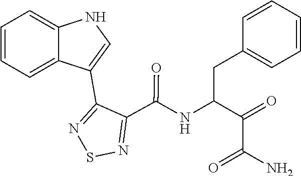 [SMILES: NC(=O)C(=O)C(Cc1ccccc1)NC(=O)c1nsnc1-c1c[nH]c2ccccc12]

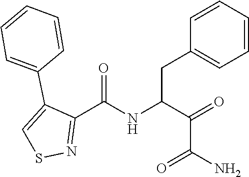 [SMILES: NC(=O)C(=O)C(Cc1ccccc1)NC(=O)c1nscc1-c1ccccc1]

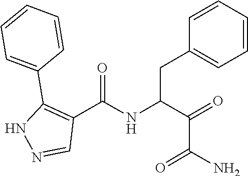 [SMILES: NC(=O)C(=O)C(Cc1ccccc1)NC(=O)c1cn[nH]c1-c1ccccc1]

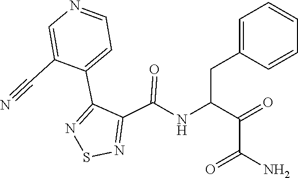 [SMILES: N#Cc1cnccc1-c1nsnc1C(=O)NC(Cc1ccccc1)C(=O)C(N)=O]

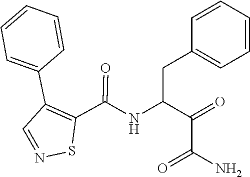 [SMILES: NC(=O)C(=O)C(Cc1ccccc1)NC(=O)c1sncc1-c1ccccc1]

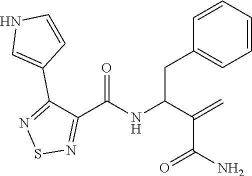

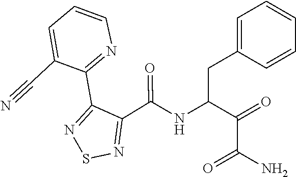 [SMILES: N#Cc1cccnc1-c1nsnc1C(=O)NC(Cc1ccccc1)C(=O)C(N)=O]

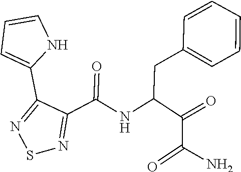 [SMILES: NC(=O)C(=O)C(Cc1ccccc1)NC(=O)c1nsnc1-c1ccc[nH]1]

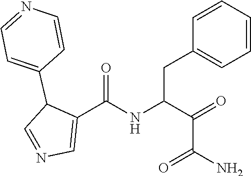 [SMILES: NC(=O)C(=O)C(Cc1ccccc1)NC(=O)C1=CN=CC1c1ccncc1]

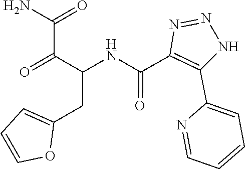 [SMILES: NC(=O)C(=O)C(Cc1ccco1)NC(=O)c1nn[nH]c1-c1ccccn1]

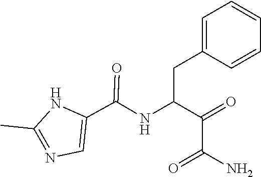 [SMILES: Cc1ncc(C(=O)NC(Cc2ccccc2)C(=O)C(N)=O)[nH]1]

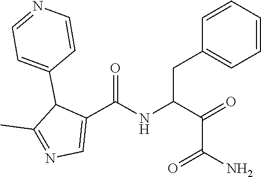 [SMILES: CC1=NC=C(C(=O)NC(Cc2ccccc2)C(=O)C(N)=O)C1c1ccncc1]

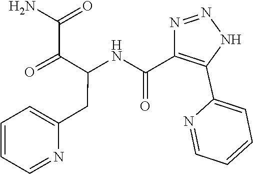 [SMILES: NC(=O)C(=O)C(Cc1ccccn1)NC(=O)c1nn[nH]c1-c1ccccn1]

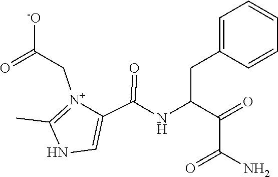

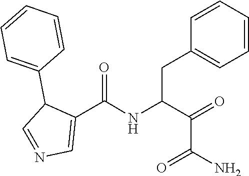 [SMILES: NC(=O)C(=O)C(Cc1ccccc1)NC(=O)C1=CN=CC1c1ccccc1]

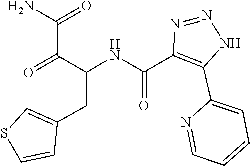 [SMILES: NC(=O)C(=O)C(Cc1ccsc1)NC(=O)c1nn[nH]c1-c1ccccn1]

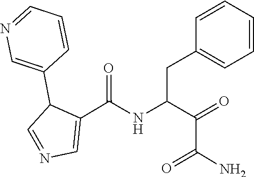 [SMILES: NC(=O)C(=O)C(Cc1ccccc1)NC(=O)C1=CN=CC1c1cccnc1]

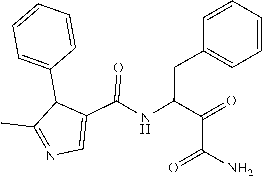 [SMILES: CC1=NC=C(C(=O)NC(Cc2ccccc2)C(=O)C(N)=O)C1c1ccccc1]

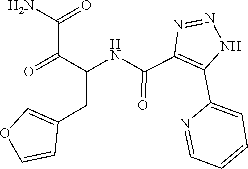 [SMILES: NC(=O)C(=O)C(Cc1ccoc1)NC(=O)c1nn[nH]c1-c1ccccn1]

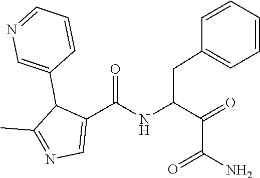 [SMILES: CC1=NC=C(C(=O)NC(Cc2ccccc2)C(=O)C(N)=O)C1c1cccnc1]

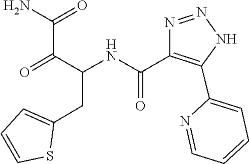 [SMILES: NC(=O)C(=O)C(Cc1cccs1)NC(=O)c1nn[nH]c1-c1ccccn1]

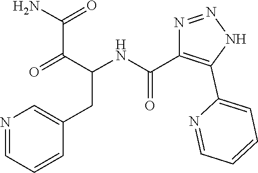 [SMILES: NC(=O)C(=O)C(Cc1cccnc1)NC(=O)c1nn[nH]c1-c1ccccn1]

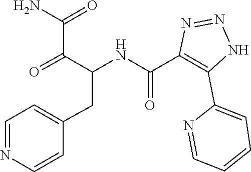 [SMILES: NC(=O)C(=O)C(Cc1ccncc1)NC(=O)c1nn[nH]c1-c1ccccn1]

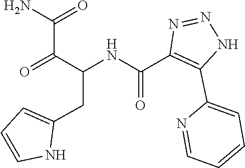 [SMILES: NC(=O)C(=O)C(Cc1ccc[nH]1)NC(=O)c1nn[nH]c1-c1ccccn1]

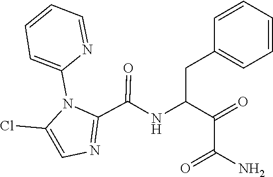 [SMILES: NC(=O)C(=O)C(Cc1ccccc1)NC(=O)c1ncc(Cl)n1-c1ccccn1]

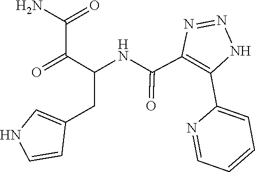 [SMILES: NC(=O)C(=O)C(Cc1cc[nH]c1)NC(=O)c1nn[nH]c1-c1ccccn1]

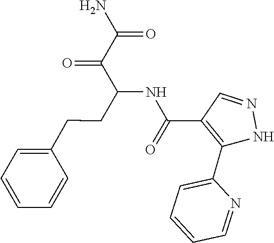 [SMILES: NC(=O)C(=O)C(CCc1ccccc1)NC(=O)c1cn[nH]c1-c1ccccn1]

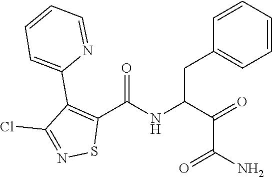 [SMILES: NC(=O)C(=O)C(Cc1ccccc1)NC(=O)c1snc(Cl)c1-c1ccccn1]

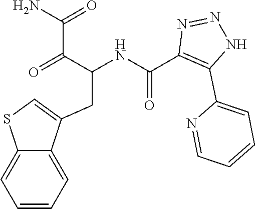 [SMILES: NC(=O)C(=O)C(Cc1csc2ccccc12)NC(=O)c1nn[nH]c1-c1ccccn1]

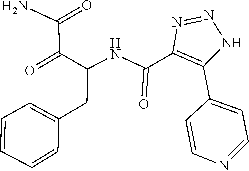 [SMILES: NC(=O)C(=O)C(Cc1ccccc1)NC(=O)c1nn[nH]c1-c1ccncc1]

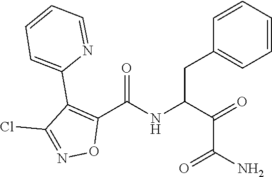 [SMILES: NC(=O)C(=O)C(Cc1ccccc1)NC(=O)c1onc(Cl)c1-c1ccccn1]

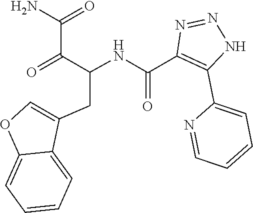 [SMILES: NC(=O)C(=O)C(Cc1coc2ccccc12)NC(=O)c1nn[nH]c1-c1ccccn1]

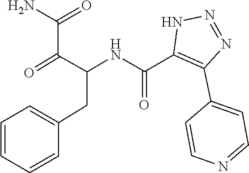 [SMILES: NC(=O)C(=O)C(Cc1ccccc1)NC(=O)c1[nH]nnc1-c1ccncc1]

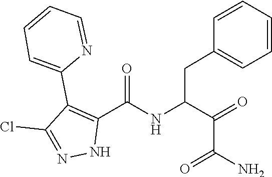 [SMILES: NC(=O)C(=O)C(Cc1ccccc1)NC(=O)c1[nH]nc(Cl)c1-c1ccccn1]

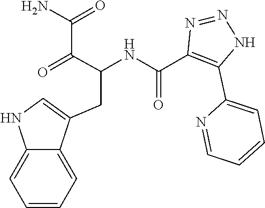 [SMILES: NC(=O)C(=O)C(Cc1c[nH]c2ccccc12)NC(=O)c1nn[nH]c1-c1ccccn1]

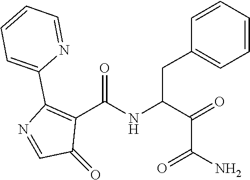 [SMILES: NC(=O)C(=O)C(Cc1ccccc1)NC(=O)C1=C(c2ccccn2)N=CC1=O]

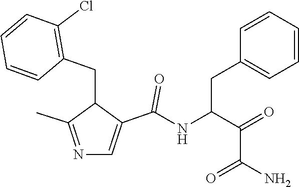 [SMILES: CC1=NC=C(C(=O)NC(Cc2ccccc2)C(=O)C(N)=O)C1Cc1ccccc1Cl]

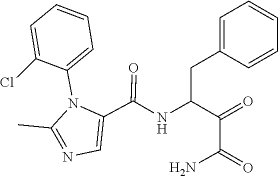 [SMILES: Cc1ncc(C(=O)NC(Cc2ccccc2)C(=O)C(N)=O)n1-c1ccccc1Cl]

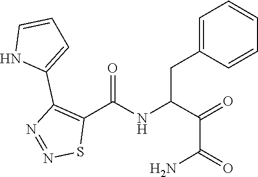 [SMILES: NC(=O)C(=O)C(Cc1ccccc1)NC(=O)c1snnc1-c1ccc[nH]1]

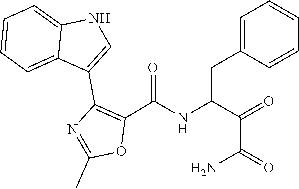 [SMILES: Cc1nc(-c2c[nH]c3ccccc23)c(C(=O)NC(Cc2ccccc2)C(=O)C(N)=O)o1]

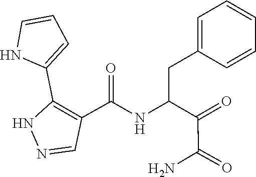 [SMILES: NC(=O)C(=O)C(Cc1ccccc1)NC(=O)c1cn[nH]c1-c1ccc[nH]1]

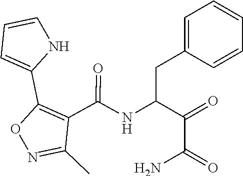 [SMILES: Cc1noc(-c2ccc[nH]2)c1C(=O)NC(Cc1ccccc1)C(=O)C(N)=O]

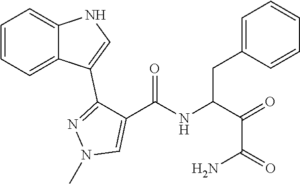

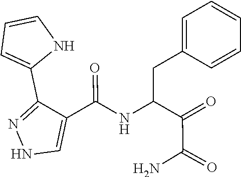

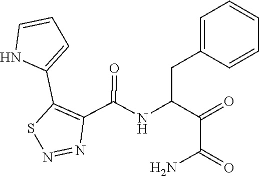 [SMILES: NC(=O)C(=O)C(Cc1ccccc1)NC(=O)c1nnsc1-c1ccc[nH]1]

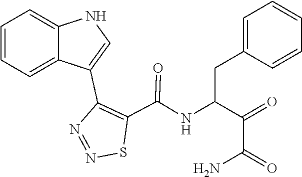

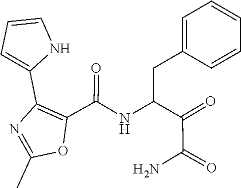 [SMILES: Cc1nc(-c2ccc[nH]2)c(C(=O)NC(Cc2ccccc2)C(=O)C(N)=O)o1]

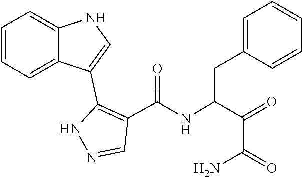 [SMILES: NC(=O)C(=O)C(Cc1ccccc1)NC(=O)c1cn[nH]c1-c1c[nH]c2ccccc12]

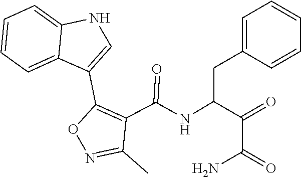 [SMILES: Cc1noc(-c2c[nH]c3ccccc23)c1C(=O)NC(Cc1ccccc1)C(=O)C(N)=O]

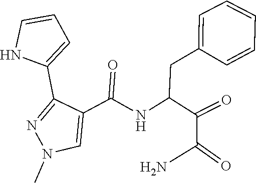

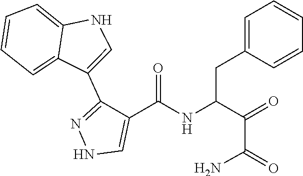

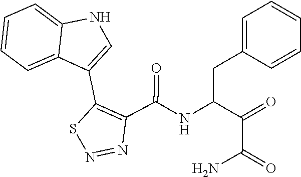 [SMILES: NC(=O)C(=O)C(Cc1ccccc1)NC(=O)c1nnsc1-c1c[nH]c2ccccc12]

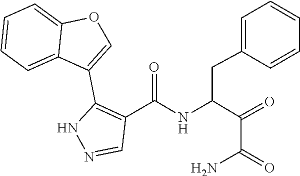 [SMILES: NC(=O)C(=O)C(Cc1ccccc1)NC(=O)c1cn[nH]c1-c1coc2ccccc12]

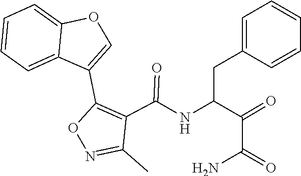 [SMILES: Cc1noc(-c2coc3ccccc23)c1C(=O)NC(Cc1ccccc1)C(=O)C(N)=O]

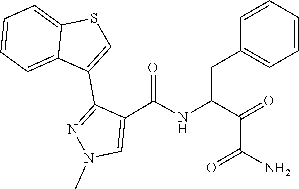 [SMILES: Cn1cc(C(=O)NC(Cc2ccccc2)C(=O)C(N)=O)c(-c2csc3ccccc23)n1]

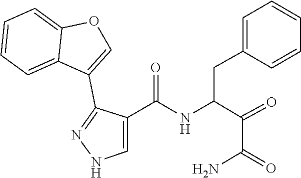 [SMILES: NC(=O)C(=O)C(Cc1ccccc1)NC(=O)c1c[nH]nc1-c1coc2ccccc12]

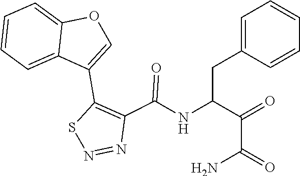 [SMILES: NC(=O)C(=O)C(Cc1ccccc1)NC(=O)c1nnsc1-c1coc2ccccc12]

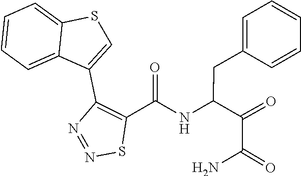

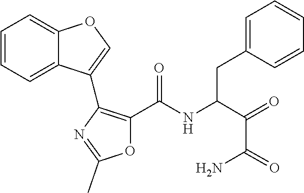 [SMILES: Cc1nc(-c2coc3ccccc23)c(C(=O)NC(Cc2ccccc2)C(=O)C(N)=O)o1]

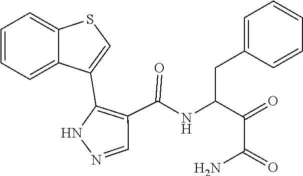 [SMILES: NC(=O)C(=O)C(Cc1ccccc1)NC(=O)c1cn[nH]c1-c1csc2ccccc12]

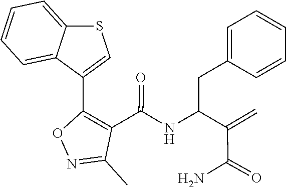 [SMILES: C=C(C(N)=O)C(Cc1ccccc1)NC(=O)c1c(C)noc1-c1csc2ccccc12]

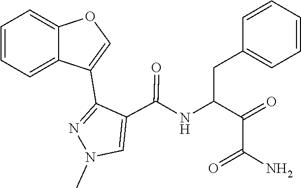 [SMILES: Cn1cc(C(=O)NC(Cc2ccccc2)C(=O)C(N)=O)c(-c2coc3ccccc23)n1]

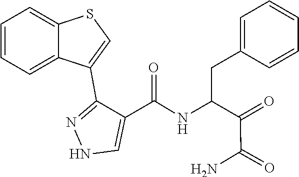

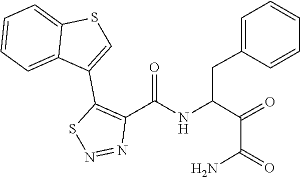 [SMILES: NC(=O)C(=O)C(Cc1ccccc1)NC(=O)c1nnsc1-c1csc2ccccc12]

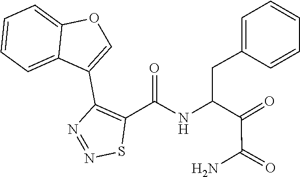 [SMILES: NC(=O)C(=O)C(Cc1ccccc1)NC(=O)c1snnc1-c1coc2ccccc12]

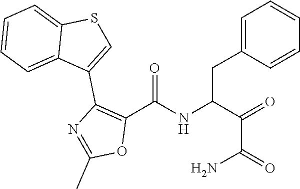 [SMILES: Cc1nc(-c2csc3ccccc23)c(C(=O)NC(Cc2ccccc2)C(=O)C(N)=O)o1]

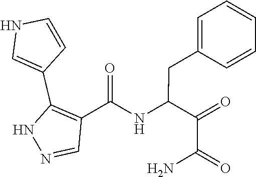 [SMILES: NC(=O)C(=O)C(Cc1ccccc1)NC(=O)c1cn[nH]c1-c1cc[nH]c1]

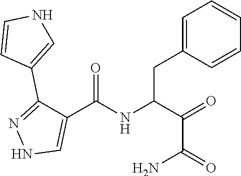

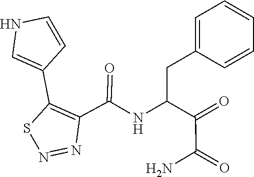 [SMILES: NC(=O)C(=O)C(Cc1ccccc1)NC(=O)c1nnsc1-c1cc[nH]c1]

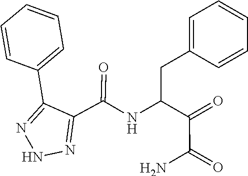 [SMILES: NC(=O)C(=O)C(Cc1ccccc1)NC(=O)c1n[nH]nc1-c1ccccc1]

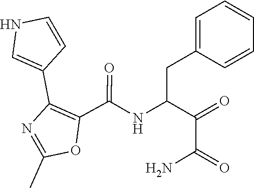 [SMILES: Cc1nc(-c2cc[nH]c2)c(C(=O)NC(Cc2ccccc2)C(=O)C(N)=O)o1]

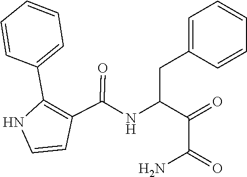 [SMILES: NC(=O)C(=O)C(Cc1ccccc1)NC(=O)c1cc[nH]c1-c1ccccc1]

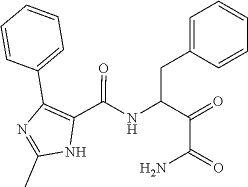 [SMILES: Cc1nc(-c2ccccc2)c(C(=O)NC(Cc2ccccc2)C(=O)C(N)=O)[nH]1]

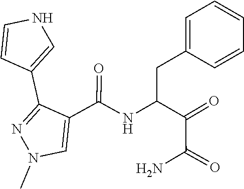 [SMILES: Cn1cc(C(=O)NC(Cc2ccccc2)C(=O)C(N)=O)c(-c2cc[nH]c2)n1]

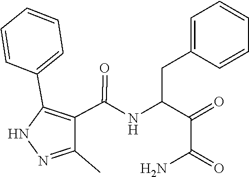 [SMILES: Cc1n[nH]c(-c2ccccc2)c1C(=O)NC(Cc1ccccc1)C(=O)C(N)=O]

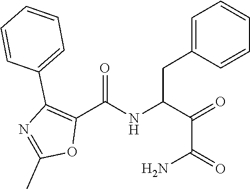 [SMILES: Cc1nc(-c2ccccc2)c(C(=O)NC(Cc2ccccc2)C(=O)C(N)=O)o1]

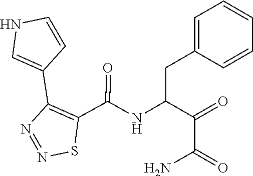 [SMILES: NC(=O)C(=O)C(Cc1ccccc1)NC(=O)c1snnc1-c1cc[nH]c1]

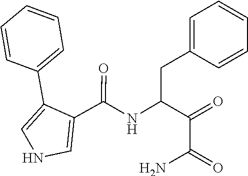 [SMILES: NC(=O)C(=O)C(Cc1ccccc1)NC(=O)c1c[nH]cc1-c1ccccc1]

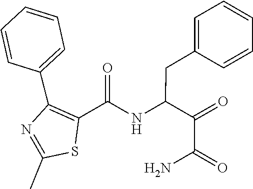 [SMILES: Cc1nc(-c2ccccc2)c(C(=O)NC(Cc2ccccc2)C(=O)C(N)=O)s1]

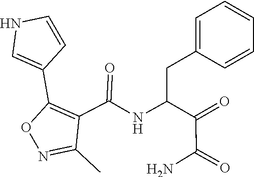 [SMILES: Cc1noc(-c2cc[nH]c2)c1C(=O)NC(Cc1ccccc1)C(=O)C(N)=O]

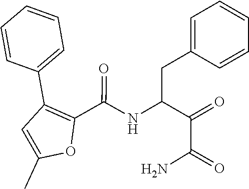 [SMILES: Cc1cc(-c2ccccc2)c(C(=O)NC(Cc2ccccc2)C(=O)C(N)=O)o1]

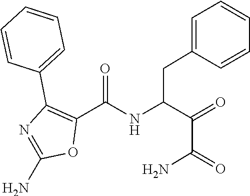 [SMILES: NC(=O)C(=O)C(Cc1ccccc1)NC(=O)c1oc(N)nc1-c1ccccc1]

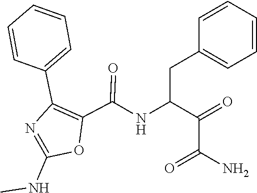 [SMILES: CNc1nc(-c2ccccc2)c(C(=O)NC(Cc2ccccc2)C(=O)C(N)=O)o1]

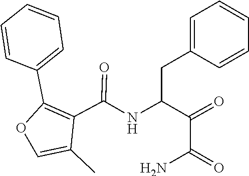 [SMILES: Cc1coc(-c2ccccc2)c1C(=O)NC(Cc1ccccc1)C(=O)C(N)=O]

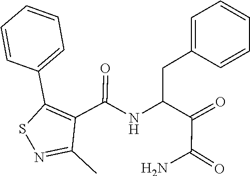 [SMILES: Cc1nsc(-c2ccccc2)c1C(=O)NC(Cc1ccccc1)C(=O)C(N)=O]

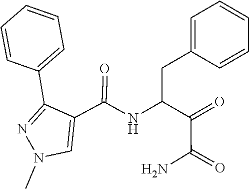 [SMILES: Cn1cc(C(=O)NC(Cc2ccccc2)C(=O)C(N)=O)c(-c2ccccc2)n1]

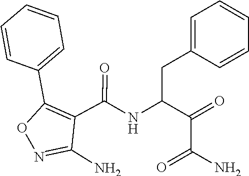 [SMILES: NC(=O)C(=O)C(Cc1ccccc1)NC(=O)c1c(N)noc1-c1ccccc1]

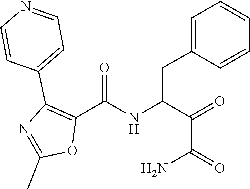 [SMILES: Cc1nc(-c2ccncc2)c(C(=O)NC(Cc2ccccc2)C(=O)C(N)=O)o1]

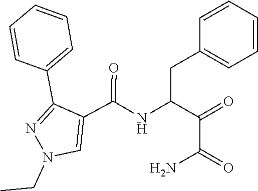 [SMILES: CCn1cc(C(=O)NC(Cc2ccccc2)C(=O)C(N)=O)c(-c2ccccc2)n1]

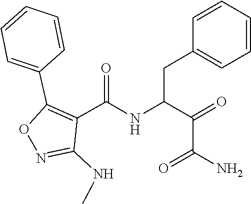 [SMILES: CNc1noc(-c2ccccc2)c1C(=O)NC(Cc1ccccc1)C(=O)C(N)=O]

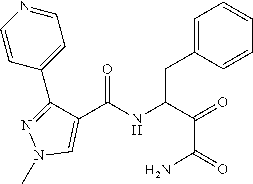

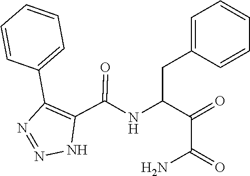 [SMILES: NC(=O)C(=O)C(Cc1ccccc1)NC(=O)c1[nH]nnc1-c1ccccc1]

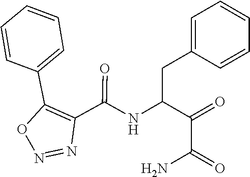 [SMILES: NC(=O)C(=O)C(Cc1ccccc1)NC(=O)c1nnoc1-c1ccccc1]

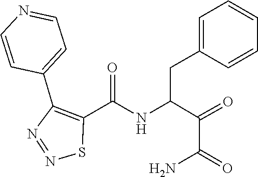

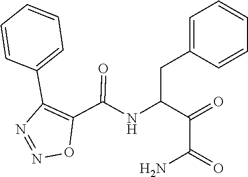

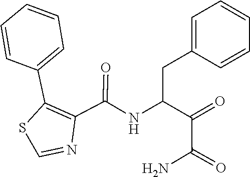 [SMILES: NC(=O)C(=O)C(Cc1ccccc1)NC(=O)c1ncsc1-c1ccccc1]

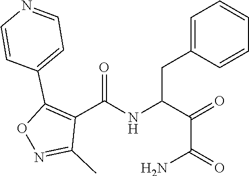 [SMILES: Cc1noc(-c2ccncc2)c1C(=O)NC(Cc1ccccc1)C(=O)C(N)=O]

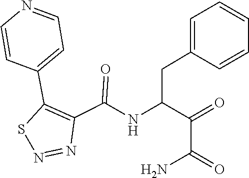 [SMILES: NC(=O)C(=O)C(Cc1ccccc1)NC(=O)c1nnsc1-c1ccncc1]

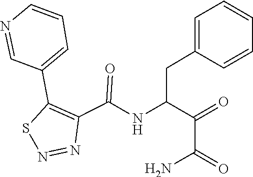 [SMILES: NC(=O)C(=O)C(Cc1ccccc1)NC(=O)c1nnsc1-c1cccnc1]

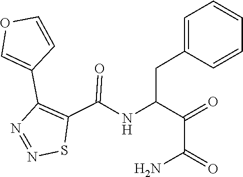 [SMILES: NC(=O)C(=O)C(Cc1ccccc1)NC(=O)c1snnc1-c1ccoc1]

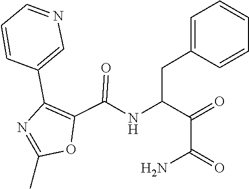 [SMILES: Cc1nc(-c2cccnc2)c(C(=O)NC(Cc2ccccc2)C(=O)C(N)=O)o1]

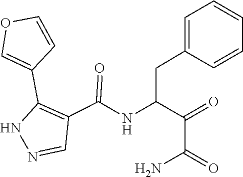 [SMILES: NC(=O)C(=O)C(Cc1ccccc1)NC(=O)c1cn[nH]c1-c1ccoc1]

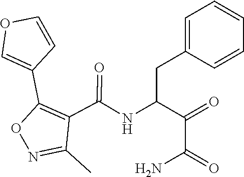 [SMILES: Cc1noc(-c2ccoc2)c1C(=O)NC(Cc1ccccc1)C(=O)C(N)=O]

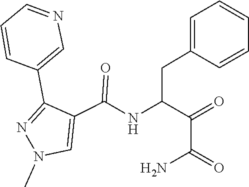 [SMILES: Cn1cc(C(=O)NC(Cc2ccccc2)C(=O)C(N)=O)c(-c2cccnc2)n1]

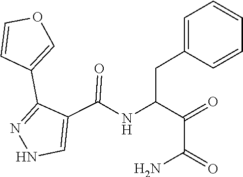 [SMILES: NC(=O)C(=O)C(Cc1ccccc1)NC(=O)c1c[nH]nc1-c1ccoc1]

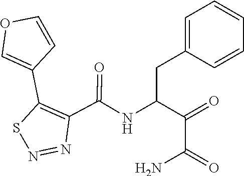 [SMILES: NC(=O)C(=O)C(Cc1ccccc1)NC(=O)c1nnsc1-c1ccoc1]

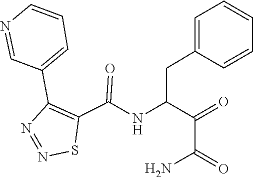 [SMILES: NC(=O)C(=O)C(Cc1ccccc1)NC(=O)c1snnc1-c1cccnc1]

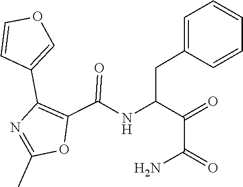 [SMILES: Cc1nc(-c2ccoc2)c(C(=O)NC(Cc2ccccc2)C(=O)C(N)=O)o1]

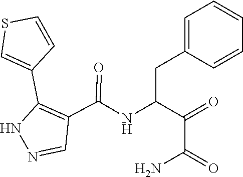 [SMILES: NC(=O)C(=O)C(Cc1ccccc1)NC(=O)c1cn[nH]c1-c1ccsc1]

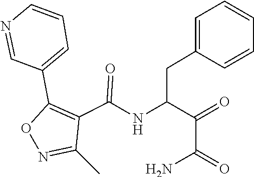 [SMILES: Cc1noc(-c2cccnc2)c1C(=O)NC(Cc1ccccc1)C(=O)C(N)=O]

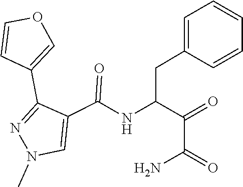 [SMILES: Cn1cc(C(=O)NC(Cc2ccccc2)C(=O)C(N)=O)c(-c2ccoc2)n1]

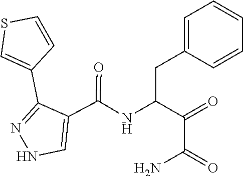 [SMILES: NC(=O)C(=O)C(Cc1ccccc1)NC(=O)c1c[nH]nc1-c1ccsc1]

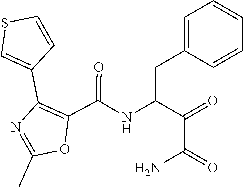 [SMILES: Cc1nc(-c2ccsc2)c(C(=O)NC(Cc2ccccc2)C(=O)C(N)=O)o1]

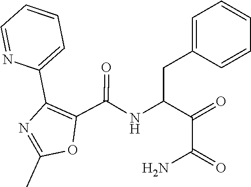 [SMILES: Cc1nc(-c2ccccn2)c(C(=O)NC(Cc2ccccc2)C(=O)C(N)=O)o1]

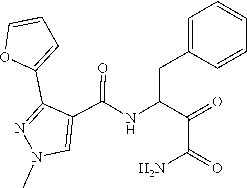 [SMILES: Cn1cc(C(=O)NC(Cc2ccccc2)C(=O)C(N)=O)c(-c2ccco2)n1]

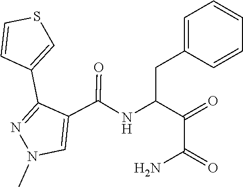 [SMILES: Cn1cc(C(=O)NC(Cc2ccccc2)C(=O)C(N)=O)c(-c2ccsc2)n1]

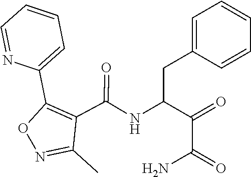 [SMILES: Cc1noc(-c2ccccn2)c1C(=O)NC(Cc1ccccc1)C(=O)C(N)=O]

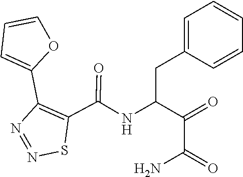 [SMILES: NC(=O)C(=O)C(Cc1ccccc1)NC(=O)c1snnc1-c1ccco1]

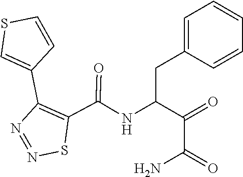 [SMILES: NC(=O)C(=O)C(Cc1ccccc1)NC(=O)c1snnc1-c1ccsc1]

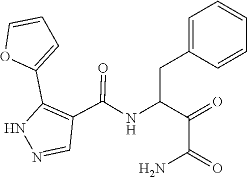 [SMILES: NC(=O)C(=O)C(Cc1ccccc1)NC(=O)c1cn[nH]c1-c1ccco1]

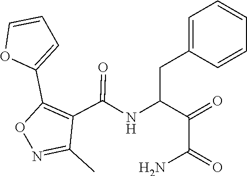 [SMILES: Cc1noc(-c2ccco2)c1C(=O)NC(Cc1ccccc1)C(=O)C(N)=O]

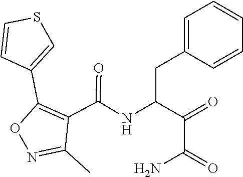 [SMILES: Cc1noc(-c2ccsc2)c1C(=O)NC(Cc1ccccc1)C(=O)C(N)=O]

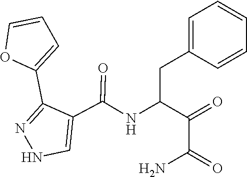 [SMILES: NC(=O)C(=O)C(Cc1ccccc1)NC(=O)c1c[nH]nc1-c1ccco1]

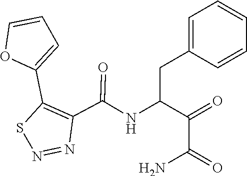 [SMILES: NC(=O)C(=O)C(Cc1ccccc1)NC(=O)c1nnsc1-c1ccco1]

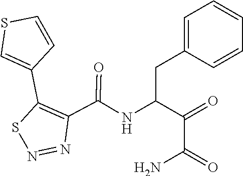 [SMILES: NC(=O)C(=O)C(Cc1ccccc1)NC(=O)c1nnsc1-c1ccsc1]

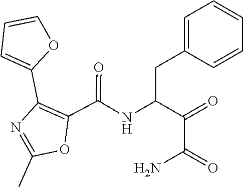 [SMILES: Cc1nc(-c2ccco2)c(C(=O)NC(Cc2ccccc2)C(=O)C(N)=O)o1]

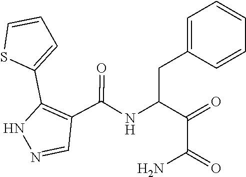 [SMILES: NC(=O)C(=O)C(Cc1ccccc1)NC(=O)c1cn[nH]c1-c1cccs1]

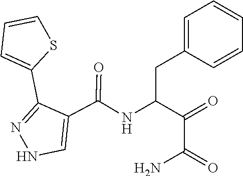 [SMILES: NC(=O)C(=O)C(Cc1ccccc1)NC(=O)c1c[nH]nc1-c1cccs1]

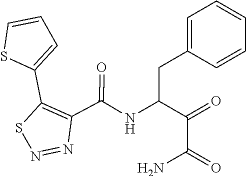 [SMILES: NC(=O)C(=O)C(Cc1ccccc1)NC(=O)c1nnsc1-c1cccs1]

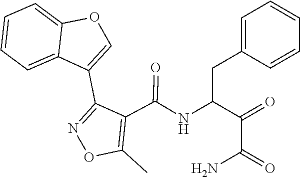 [SMILES: Cc1onc(-c2coc3ccccc23)c1C(=O)NC(Cc1ccccc1)C(=O)C(N)=O]

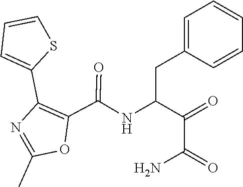 [SMILES: Cc1nc(-c2cccs2)c(C(=O)NC(Cc2ccccc2)C(=O)C(N)=O)o1]

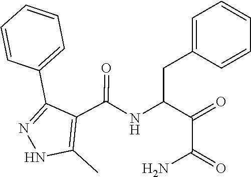 [SMILES: Cc1[nH]nc(-c2ccccc2)c1C(=O)NC(Cc1ccccc1)C(=O)C(N)=O]

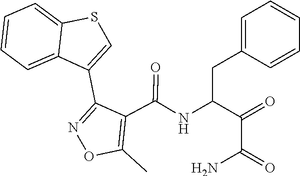 [SMILES: Cc1onc(-c2csc3ccccc23)c1C(=O)NC(Cc1ccccc1)C(=O)C(N)=O]

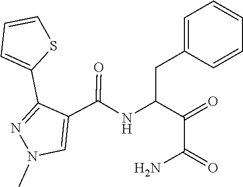 [SMILES: Cn1cc(C(=O)NC(Cc2ccccc2)C(=O)C(N)=O)c(-c2cccs2)n1]

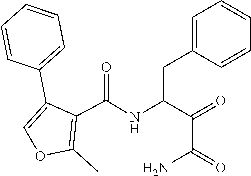 [SMILES: Cc1occ(-c2ccccc2)c1C(=O)NC(Cc1ccccc1)C(=O)C(N)=O]

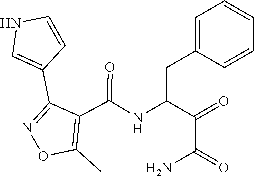 [SMILES: Cc1onc(-c2cc[nH]c2)c1C(=O)NC(Cc1ccccc1)C(=O)C(N)=O]

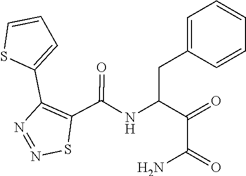 [SMILES: NC(=O)C(=O)C(Cc1ccccc1)NC(=O)c1snnc1-c1cccs1]

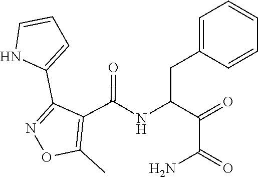 [SMILES: Cc1onc(-c2ccc[nH]2)c1C(=O)NC(Cc1ccccc1)C(=O)C(N)=O]

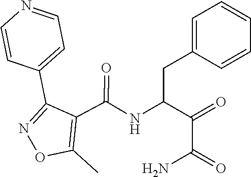 [SMILES: Cc1onc(-c2ccncc2)c1C(=O)NC(Cc1ccccc1)C(=O)C(N)=O]

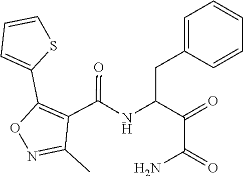 [SMILES: Cc1noc(-c2cccs2)c1C(=O)NC(Cc1ccccc1)C(=O)C(N)=O]

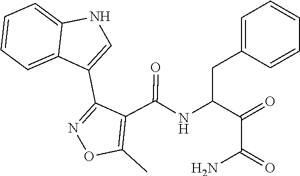 [SMILES: Cc1onc(-c2c[nH]c3ccccc23)c1C(=O)NC(Cc1ccccc1)C(=O)C(N)=O]

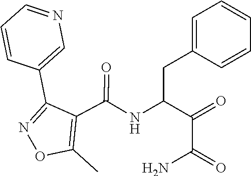 [SMILES: Cc1onc(-c2cccnc2)c1C(=O)NC(Cc1ccccc1)C(=O)C(N)=O]

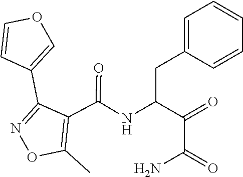 [SMILES: Cc1onc(-c2ccoc2)c1C(=O)NC(Cc1ccccc1)C(=O)C(N)=O]

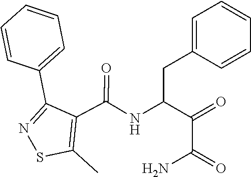 [SMILES: Cc1snc(-c2ccccc2)c1C(=O)NC(Cc1ccccc1)C(=O)C(N)=O]

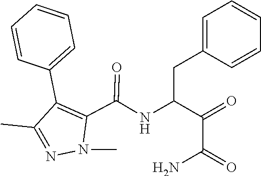 [SMILES: Cc1nn(C)c(C(=O)NC(Cc2ccccc2)C(=O)C(N)=O)c1-c1ccccc1]

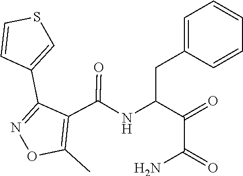 [SMILES: Cc1onc(-c2ccsc2)c1C(=O)NC(Cc1ccccc1)C(=O)C(N)=O]

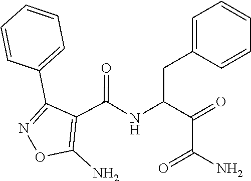 [SMILES: NC(=O)C(=O)C(Cc1ccccc1)NC(=O)c1c(-c2ccccc2)noc1N]

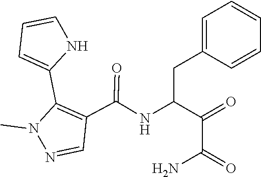 [SMILES: Cn1ncc(C(=O)NC(Cc2ccccc2)C(=O)C(N)=O)c1-c1ccc[nH]1]

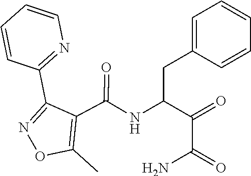 [SMILES: Cc1onc(-c2ccccn2)c1C(=O)NC(Cc1ccccc1)C(=O)C(N)=O]

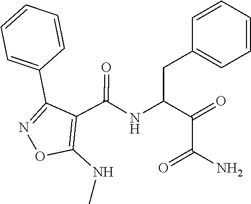 [SMILES: CNc1onc(-c2ccccc2)c1C(=O)NC(Cc1ccccc1)C(=O)C(N)=O]

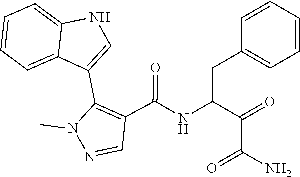 [SMILES: Cn1ncc(C(=O)NC(Cc2ccccc2)C(=O)C(N)=O)c1-c1c[nH]c2ccccc12]

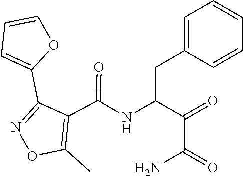 [SMILES: Cc1onc(-c2ccco2)c1C(=O)NC(Cc1ccccc1)C(=O)C(N)=O]

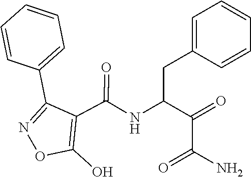 [SMILES: NC(=O)C(=O)C(Cc1ccccc1)NC(=O)c1c(-c2ccccc2)noc1O]

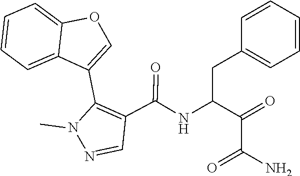 [SMILES: Cn1ncc(C(=O)NC(Cc2ccccc2)C(=O)C(N)=O)c1-c1coc2ccccc12]

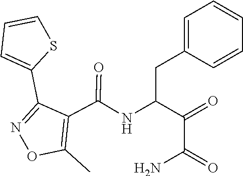 [SMILES: Cc1onc(-c2cccs2)c1C(=O)NC(Cc1ccccc1)C(=O)C(N)=O]

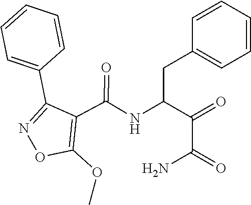 [SMILES: COc1onc(-c2ccccc2)c1C(=O)NC(Cc1ccccc1)C(=O)C(N)=O]

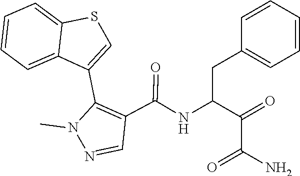 [SMILES: Cn1ncc(C(=O)NC(Cc2ccccc2)C(=O)C(N)=O)c1-c1csc2ccccc12]

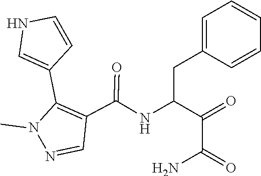 [SMILES: Cn1ncc(C(=O)NC(Cc2ccccc2)C(=O)C(N)=O)c1-c1cc[nH]c1]

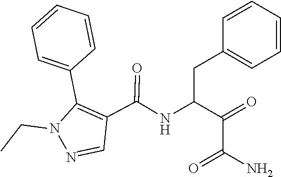 [SMILES: CCn1ncc(C(=O)NC(Cc2ccccc2)C(=O)C(N)=O)c1-c1ccccc1]

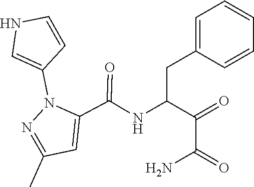 [SMILES: Cc1cc(C(=O)NC(Cc2ccccc2)C(=O)C(N)=O)n(-c2cc[nH]c2)n1]

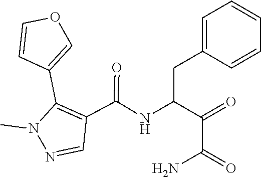 [SMILES: Cn1ncc(C(=O)NC(Cc2ccccc2)C(=O)C(N)=O)c1-c1ccoc1]

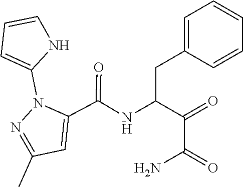 [SMILES: Cc1cc(C(=O)NC(Cc2ccccc2)C(=O)C(N)=O)n(-c2ccc[nH]2)n1]

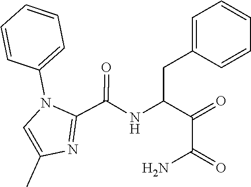 [SMILES: Cc1cn(-c2ccccc2)c(C(=O)NC(Cc2ccccc2)C(=O)C(N)=O)n1]

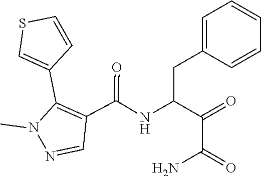 [SMILES: Cn1ncc(C(=O)NC(Cc2ccccc2)C(=O)C(N)=O)c1-c1ccsc1]

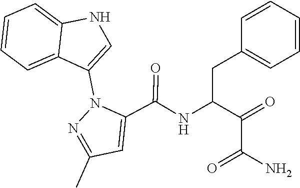 [SMILES: Cc1cc(C(=O)NC(Cc2ccccc2)C(=O)C(N)=O)n(-c2c[nH]c3ccccc23)n1]

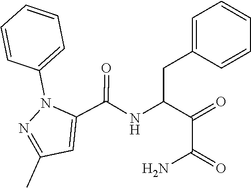 [SMILES: Cc1cc(C(=O)NC(Cc2ccccc2)C(=O)C(N)=O)n(-c2ccccc2)n1]

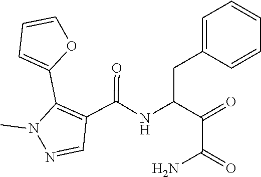 [SMILES: Cn1ncc(C(=O)NC(Cc2ccccc2)C(=O)C(N)=O)c1-c1ccco1]

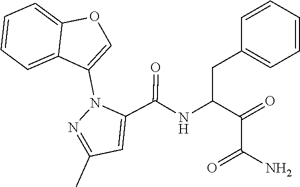 [SMILES: Cc1cc(C(=O)NC(Cc2ccccc2)C(=O)C(N)=O)n(-c2coc3ccccc23)n1]

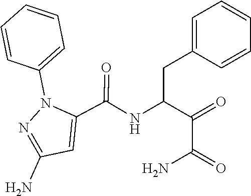 [SMILES: NC(=O)C(=O)C(Cc1ccccc1)NC(=O)c1cc(N)nn1-c1ccccc1]

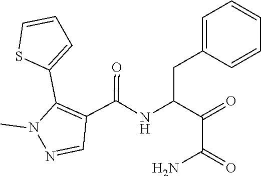 [SMILES: Cn1ncc(C(=O)NC(Cc2ccccc2)C(=O)C(N)=O)c1-c1cccs1]

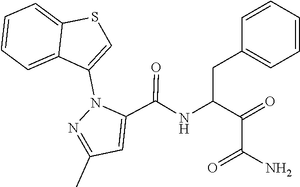 [SMILES: Cc1cc(C(=O)NC(Cc2ccccc2)C(=O)C(N)=O)n(-c2csc3ccccc23)n1]

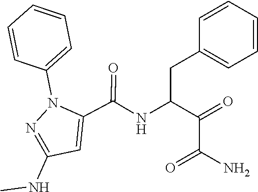 [SMILES: CNc1cc(C(=O)NC(Cc2ccccc2)C(=O)C(N)=O)n(-c2ccccc2)n1]

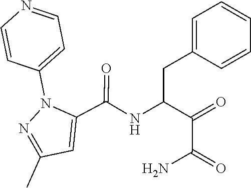 [SMILES: Cc1cc(C(=O)NC(Cc2ccccc2)C(=O)C(N)=O)n(-c2ccncc2)n1]

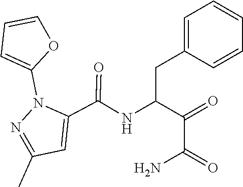 [SMILES: Cc1cc(C(=O)NC(Cc2ccccc2)C(=O)C(N)=O)n(-c2ccco2)n1]

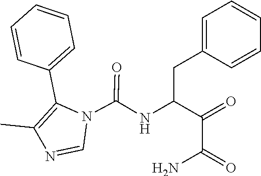 [SMILES: Cc1ncn(C(=O)NC(Cc2ccccc2)C(=O)C(N)=O)c1-c1ccccc1]

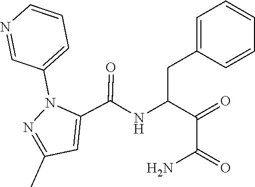 [SMILES: Cc1cc(C(=O)NC(Cc2ccccc2)C(=O)C(N)=O)n(-c2cccnc2)n1]

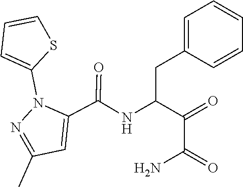 [SMILES: Cc1cc(C(=O)NC(Cc2ccccc2)C(=O)C(N)=O)n(-c2cccs2)n1]

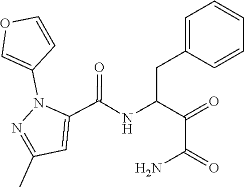 [SMILES: Cc1cc(C(=O)NC(Cc2ccccc2)C(=O)C(N)=O)n(-c2ccoc2)n1]

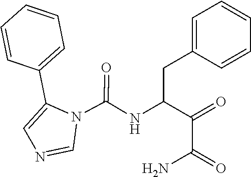 [SMILES: NC(=O)C(=O)C(Cc1ccccc1)NC(=O)n1cncc1-c1ccccc1]

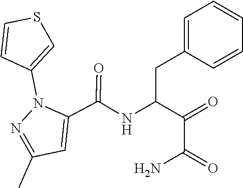 [SMILES: Cc1cc(C(=O)NC(Cc2ccccc2)C(=O)C(N)=O)n(-c2ccsc2)n1]

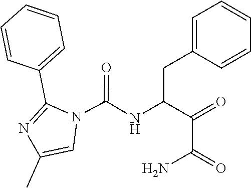 [SMILES: Cc1cn(C(=O)NC(Cc2ccccc2)C(=O)C(N)=O)c(-c2ccccc2)n1]

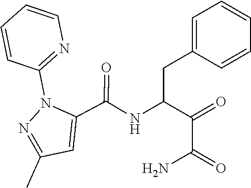 [SMILES: Cc1cc(C(=O)NC(Cc2ccccc2)C(=O)C(N)=O)n(-c2ccccn2)n1]

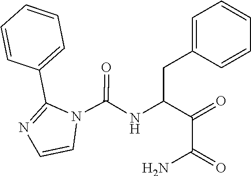 [SMILES: NC(=O)C(=O)C(Cc1ccccc1)NC(=O)n1ccnc1-c1ccccc1]

The compounds of Formulas I-III and/or Tables 1a and 1b may be prepared according to the methods described in International Pub. No. WO 2018/064119 the entirety of which is incorporated by reference herein.

In some embodiments provided herein, the calpain inhibitor may be a compound having the structure of Formula IV:

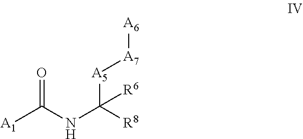

or a pharmaceutically acceptable salt thereof, wherein:

A₁ is selected from the group consisting of substituted C₆₋₁₀ aryl, optionally substituted 9-14 membered heteroaryl, optionally substituted 9-14 membered heterocyclyl, and optionally substituted 9-14 membered carbocyclyl,

wherein when A₁ is a substituted C₆₋₁₀ aryl; the aryl is substituted with one or more moieties selected from the group consisting of C₁, F, Br, Ph, acetylene, cyclopropyl, CN, hydroxy, phenyl, C₁₋₄ alkyl optionally substituted with halo, and C₁-C₆ alkoxy optionally substituted with halo;

A₅ is selected from the group consisting of optionally substituted 3-10 membered heterocyclyl, optionally substituted C₆₋₁₀ aryl, optionally substituted 5-10 membered heteroaryl, optionally substituted C₃₋₁₀ carbocyclyl, optionally substituted C₁₋₈ alkyl, —S—, —S(═O)—, —SO₂—, —O—, —C(═S)—, —C(═O)—, —NR—, —CH═CH—, —OC(O)NH—, —NHC(O)NH—, —NHC(O)O—, —NHC(O)—, —NHC(S)NH—, —NHC(S)O—, —NHC(S)—, and single bond;

A₆ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₈ alkyl, optionally substituted —O—C₁₋₆ alkyl, optionally substituted —O C₂₋₆ alkenyl, and any natural or non-natural amino acid side chain;

A₇ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₈ alkyl, —S—, S(═O)—, —SO₂—, —O—, —C(═S)—, —C(═O)—, —NR—, —CH═CH—, —OC(O)NH—, —NHC(O)NH—, —NHC(O)O—, —NHC(O)—, —NHC(S)NH—, —NHC(S)O—, —NHC(S)—, and single bond;

when A₅ and A₇ are single bond, A₆ is directly attached to the carbon to which R⁸ is attached;

R⁸ is selected from the group consisting of —COR¹, —CN, —CH═CHSO₂R, —CH₂NO₂;

R¹ is selected from the group consisting of H, —OH, C₁₋₄ haloalkyl, —COOH, —CH₂NO₂, —C(═O)NOR, —NH₂, —CONR²R³, —CH(CH₃)═CH₂, —CH(CF₃)NR²R³, —C(F)═CHCH₂CH₃,

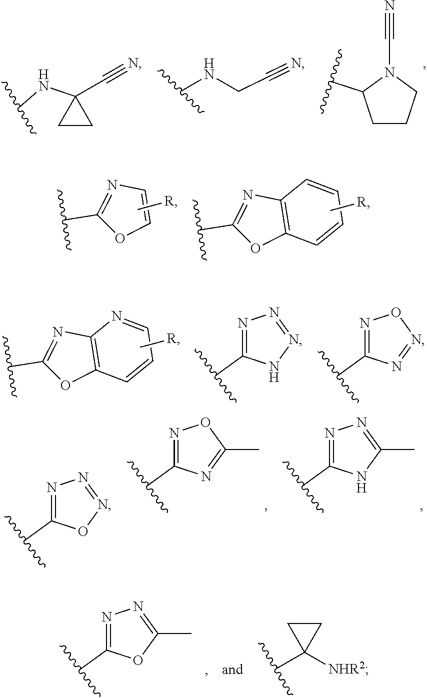

and

each R, R², and R³ are independently selected from —H, C₁₋₄ alkyl optionally substituted with one or more R¹³, optionally substituted C₃₋₇ carbocyclyl, optionally substituted 5-10 membered heterocyclyl, optionally substituted C₆₋₁₀ aryl, and optionally substituted 5-10 membered heteroaryl; and

R⁶ is independently selected from —H and optionally substituted C₁₋₄ alkyl; and

R¹³ is independently selected from C₁-C₆ alkyl, C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 5-10 membered heterocyclyl (optionally substituted with halo, C₁-C₆ alkyl, C₁-C₆ alkoxy, C₁-C₆ haloalkyl, and C₁-C₆ haloalkoxy), 5-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), aryl(C₁-C₆)alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with halo, C₁-C₆ alkyl, C₁-C₆ alkoxy, C₁-C₆ haloalkyl, and C₁-C₆ haloalkoxy), halo, cyano, hydroxy, C₁-C₆ alkoxy, C₁-C₆ alkoxy(C₁-C₆)alkyl (i.e., ether), aryloxy, sulfhydryl (mercapto), halo(C₁-C₆)alkyl (e.g., —CF₃), halo(C₁-C₆)alkoxy (e.g., —OCF₃), C₁-C₆ alkylthio, arylthio, amino, amino(C₁-C₆)alkyl, nitro, O-carbamyl, N-carbamyl, O-thiocarbamyl, N-thiocarbamyl, C-amido, N-amido, S-sulfonamido, C-carboxy, O-carboxy, acyl, cyanato, isocyanato, thiocyanato, isothiocyanato, sulfinyl, sulfonyl, and oxo (═O).

In some embodiments, the calpain inhibitor can be selected from the group consisting of the compounds listed in Table 2 below, or pharmaceutically acceptable salts thereof.

TABLE 2

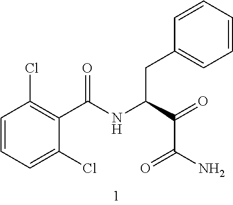 [SMILES: NC(=O)C(=O)[C@H](Cc1ccccc1)NC(=O)c1c(Cl)cccc1Cl]

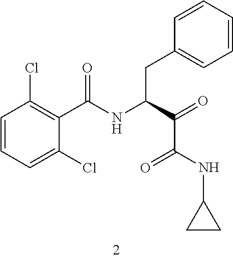 [SMILES: O=C(NC1CC1)C(=O)[C@H](Cc1ccccc1)NC(=O)c1c(Cl)cccc1Cl]

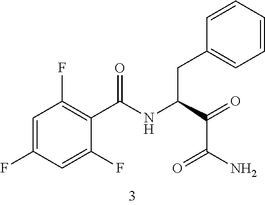 [SMILES: NC(=O)C(=O)[C@H](Cc1ccccc1)NC(=O)c1c(F)cc(F)cc1F]

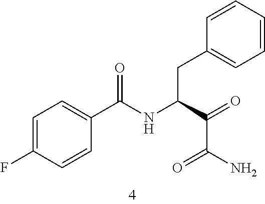 [SMILES: NC(=O)C(=O)[C@H](Cc1ccccc1)NC(=O)c1ccc(F)cc1]

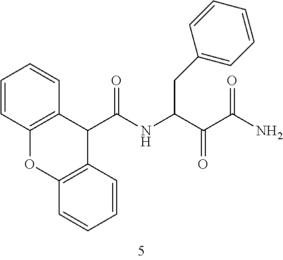 [SMILES: NC(=O)C(=O)C(Cc1ccccc1)NC(=O)C1c2ccccc2Oc2ccccc21]

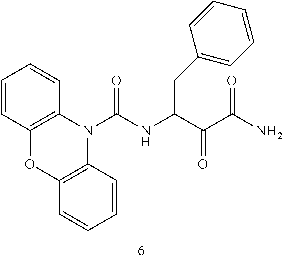 [SMILES: NC(=O)C(=O)C(Cc1ccccc1)NC(=O)N1c2ccccc2Oc2ccccc21]

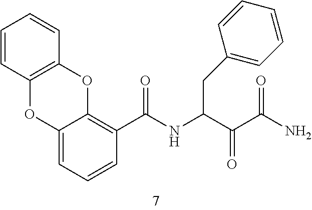 [SMILES: NC(=O)C(=O)C(Cc1ccccc1)NC(=O)c1cccc2c1Oc1ccccc1O2]

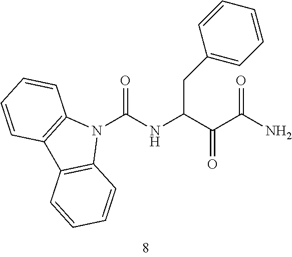 [SMILES: NC(=O)C(=O)C(Cc1ccccc1)NC(=O)n1c2ccccc2c2ccccc21]

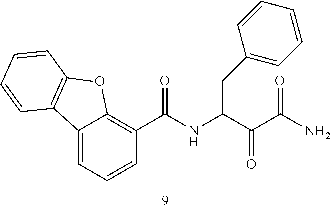 [SMILES: NC(=O)C(=O)C(Cc1ccccc1)NC(=O)c1cccc2c1oc1ccccc12]

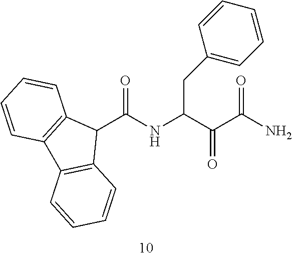 [SMILES: NC(=O)C(=O)C(Cc1ccccc1)NC(=O)C1c2ccccc2-c2ccccc21]

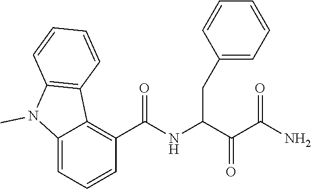 [SMILES: Cn1c2ccccc2c2c(C(=O)NC(Cc3ccccc3)C(=O)C(N)=O)cccc21]

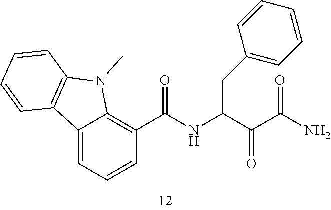 [SMILES: Cn1c2ccccc2c2cccc(C(=O)NC(Cc3ccccc3)C(=O)C(N)=O)c21]

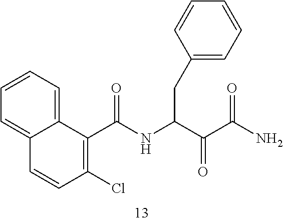 [SMILES: NC(=O)C(=O)C(Cc1ccccc1)NC(=O)c1c(Cl)ccc2ccccc12]

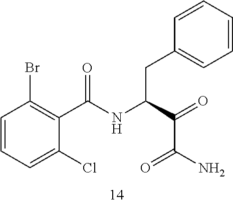 [SMILES: NC(=O)C(=O)[C@H](Cc1ccccc1)NC(=O)c1c(Cl)cccc1Br]

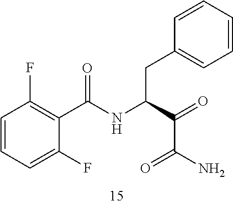 [SMILES: NC(=O)C(=O)[C@H](Cc1ccccc1)NC(=O)c1c(F)cccc1F]

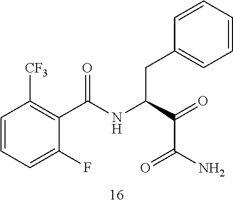 [SMILES: NC(=O)C(=O)[C@H](Cc1ccccc1)NC(=O)c1c(F)cccc1C(F)(F)F]

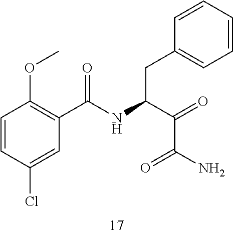 [SMILES: COc1ccc(Cl)cc1C(=O)N[C@@H](Cc1ccccc1)C(=O)C(N)=O]

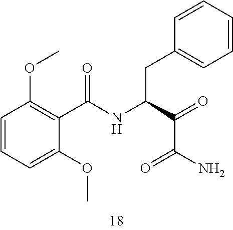 [SMILES: COc1cccc(OC)c1C(=O)N[C@@H](Cc1ccccc1)C(=O)C(N)=O]

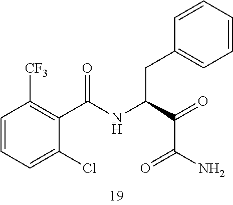 [SMILES: NC(=O)C(=O)[C@H](Cc1ccccc1)NC(=O)c1c(Cl)cccc1C(F)(F)F]

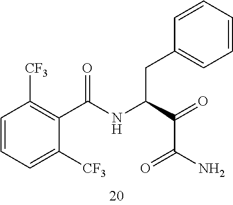 [SMILES: NC(=O)C(=O)[C@H](Cc1ccccc1)NC(=O)c1c(C(F)(F)F)cccc1C(F)(F)F]

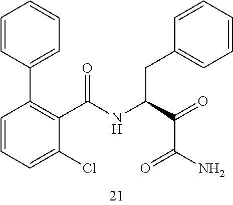 [SMILES: NC(=O)C(=O)[C@H](Cc1ccccc1)NC(=O)c1c(Cl)cccc1-c1ccccc1]

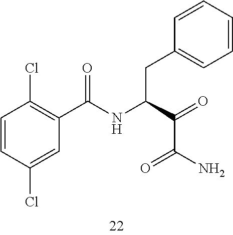 [SMILES: NC(=O)C(=O)[C@H](Cc1ccccc1)NC(=O)c1cc(Cl)ccc1Cl]

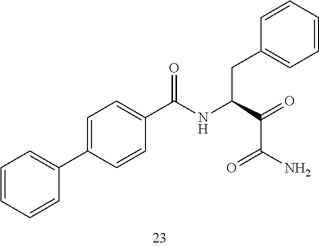 [SMILES: NC(=O)C(=O)[C@H](Cc1ccccc1)NC(=O)c1ccc(-c2ccccc2)cc1]

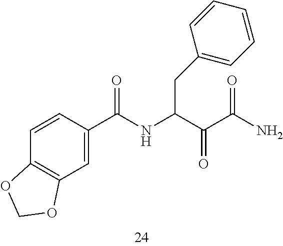 [SMILES: NC(=O)C(=O)C(Cc1ccccc1)NC(=O)c1ccc2c(c1)OCO2]

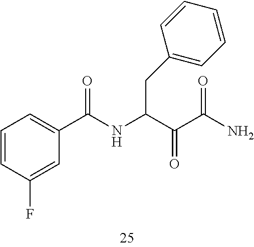 [SMILES: NC(=O)C(=O)C(Cc1ccccc1)NC(=O)c1cccc(F)c1]

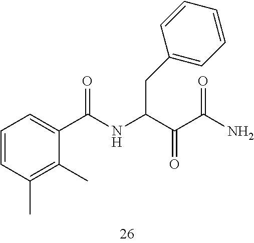 [SMILES: Cc1cccc(C(=O)NC(Cc2ccccc2)C(=O)C(N)=O)c1C]

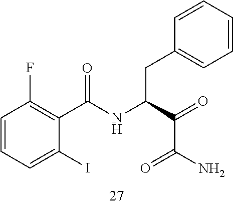 [SMILES: NC(=O)C(=O)[C@H](Cc1ccccc1)NC(=O)c1c(F)cccc1I]

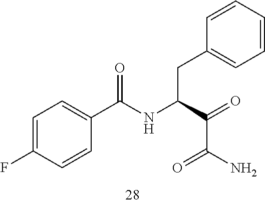 [SMILES: NC(=O)C(=O)[C@H](Cc1ccccc1)NC(=O)c1ccc(F)cc1]

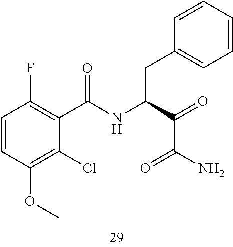 [SMILES: COc1ccc(F)c(C(=O)N[C@@H](Cc2ccccc2)C(=O)C(N)=O)c1Cl]

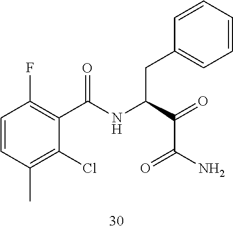 [SMILES: Cc1ccc(F)c(C(=O)N[C@@H](Cc2ccccc2)C(=O)C(N)=O)c1Cl]

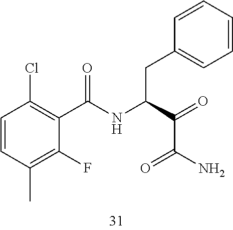 [SMILES: Cc1ccc(Cl)c(C(=O)N[C@@H](Cc2ccccc2)C(=O)C(N)=O)c1F]

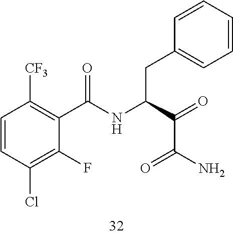 [SMILES: NC(=O)C(=O)[C@H](Cc1ccccc1)NC(=O)c1c(C(F)(F)F)ccc(Cl)c1F]

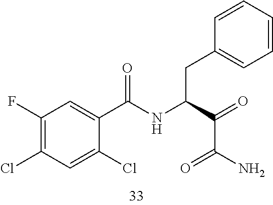 [SMILES: NC(=O)C(=O)[C@H](Cc1ccccc1)NC(=O)c1cc(F)c(Cl)cc1Cl]

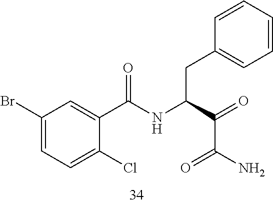 [SMILES: NC(=O)C(=O)[C@H](Cc1ccccc1)NC(=O)c1cc(Br)ccc1Cl]

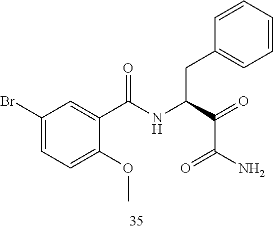 [SMILES: COc1ccc(Br)cc1C(=O)N[C@@H](Cc1ccccc1)C(=O)C(N)=O]

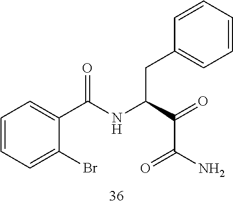 [SMILES: NC(=O)C(=O)[C@H](Cc1ccccc1)NC(=O)c1ccccc1Br]

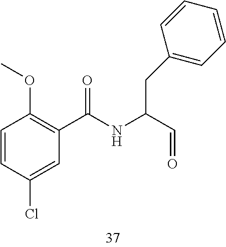 [SMILES: COc1ccc(Cl)cc1C(=O)NC(C=O)Cc1ccccc1]

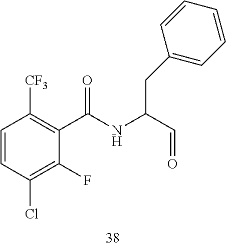 [SMILES: O=CC(Cc1ccccc1)NC(=O)c1c(C(F)(F)F)ccc(Cl)c1F]

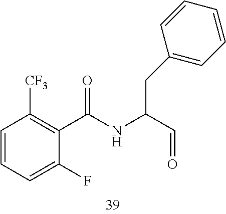 [SMILES: O=CC(Cc1ccccc1)NC(=O)c1c(F)cccc1C(F)(F)F]

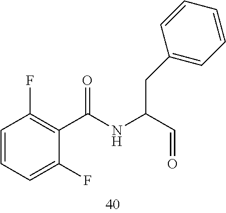 [SMILES: O=CC(Cc1ccccc1)NC(=O)c1c(F)cccc1F]

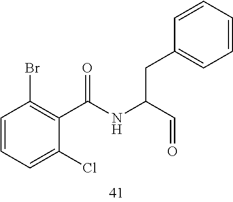 [SMILES: O=CC(Cc1ccccc1)NC(=O)c1c(Cl)cccc1Br]

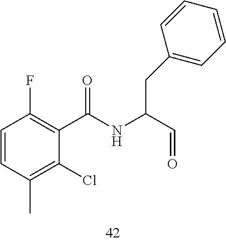 [SMILES: Cc1ccc(F)c(C(=O)NC(C=O)Cc2ccccc2)c1Cl]

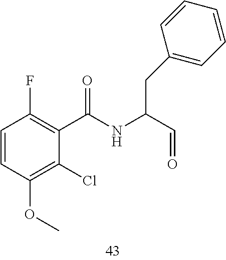 [SMILES: COc1ccc(F)c(C(=O)NC(C=O)Cc2ccccc2)c1Cl]

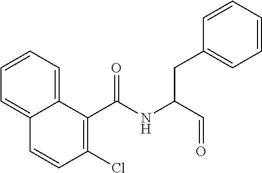 [SMILES: O=CC(Cc1ccccc1)NC(=O)c1c(Cl)ccc2ccccc12]

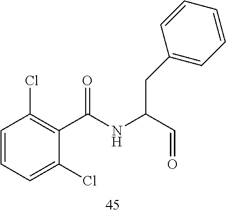 [SMILES: O=CC(Cc1ccccc1)NC(=O)c1c(Cl)cccc1Cl]

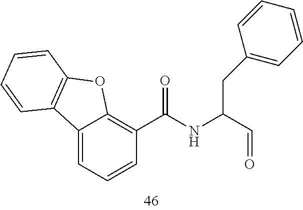 [SMILES: O=CC(Cc1ccccc1)NC(=O)c1cccc2c1oc1ccccc12]

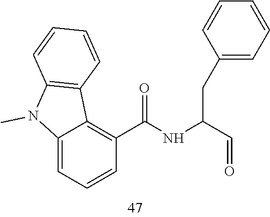 [SMILES: Cn1c2ccccc2c2c(C(=O)NC(C=O)Cc3ccccc3)cccc21]

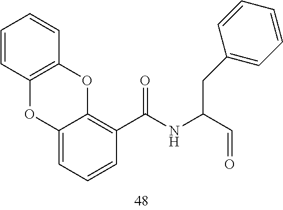 [SMILES: O=CC(Cc1ccccc1)NC(=O)c1cccc2c1Oc1ccccc1O2]

or a pharmaceutically acceptable salt thereof.

The compounds of Formula IV and/or Table 2 may be prepared according to the methods described in the Examples provided herein.

In some embodiments, the calpain inhibitor may be a compound having the structure of Formula V:

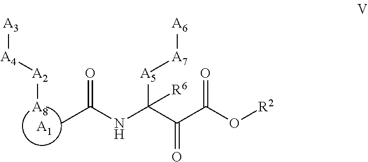

or a pharmaceutically acceptable salt thereof, wherein:

A₁ is selected from the group consisting of optionally substituted 5-10 membered heterocyclyl; optionally substituted 5-, 8-, or 9-membered heteroaryl; and optionally substituted C₃₋₁₀ carbocyclyl;

A₂ is selected from the group consisting of optionally substituted 3-10 membered heterocyclyl, optionally substituted C₆₋₁₀ aryl, optionally substituted 5-10 membered heteroaryl, optionally substituted C₃₋₁₀ carbocyclyl, —CR₂—, —S—, —S(═O)—, —SO₂—, —O—, —C(═S)—, —C(═O)—, —NR—, —CH═CH—, —C≡C—, —OC(O)NH—, —NHC(O)NH—, —NHC(O)O—, —NHC(O)—, —NHC(S)NH—, —NHC(S)O—, —NHC(S)—, and single bond;

A₄ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₄ alkyl, —(CR₂)ₙ—S—(CR₂)ₙ—, —(CR₂)ₙ—S(═O)—(CR₂)ₙ—, —(CR₂)ₙ—SO₂—(CR₂)ₙ—, —(CR₂)ₙ—O—(CR₂)ₙ—, —(CR₂)ₙ—C(═S)—(CR₂)ₙ—, —(CR₂)ₙ—C(═O)—(CR₂)ₙ—, —(CR₂)ₙ—NR—(CR₂)ₙ—, —(CR₂)ₙ—CH═CH—(CR₂)ₙ—, —(CR₂)ₙ—OC(O)NH—(CR₂)ₙ—, —(CR₂)ₙ—NHC(O)NH—(CR₂)ₙ—, —(CR₂)ₙ—NHC(O)O—(CR₂)ₙ—, —(CR₂)ₙ—NHC(O)—(CR₂)ₙ—, —(CR₂)ₙ—NHC(S)NH—(CR₂)ₙ—, —(CR₂)ₙ—NHC(S)O—(CR₂)ₙ—, —(CR₂)ₙ—NHC(S)—(CR₂)ₙ—, and single bond;

when A₂ and A₄ are single bond, A₃ is directly attached to A₈;

A₃ is selected from the group consisting of optionally substituted C₆₋₁₀ aryl, optionally substituted 5-10 membered heteroaryl, optionally substituted 3-10 membered heterocyclyl, and optionally substituted C₃₋₁₀ carbocyclyl, or if A₂ is selected from optionally substituted 3-10 membered heterocyclyl, optionally substituted C₆₋₁₀ aryl, optionally substituted 5-10 membered heteroaryl, and optionally substituted C₃₋₁₀ carbocyclyl, then A₃ is selected from the group consisting of hydrogen, optionally substituted C₆₋₁₀ aryl, optionally substituted 5-10 membered heteroaryl, optionally substituted 3-10 membered heterocyclyl, optionally substituted C₃₋₁₀ carbocyclyl, —C≡CH, and optionally substituted 2- to 5-membered polyethylene glycol;

A₅ is selected from the group consisting of optionally substituted 3-10 membered heterocyclyl, optionally substituted C₆₋₁₀ aryl, optionally substituted 5-10 membered heteroaryl, optionally substituted C₃₋₁₀ carbocyclyl, optionally substituted C₁₋₈ alkyl, —S—, —S(═O)—, —SO₂—, —O—, —C(═S)—, —C(═O)—, —NR—, —CH═CH—, —OC(O)NH—, —NHC(O)NH—, —NHC(O)O—, —NHC(O)—, —NHC(S)NH—, —NHC(S)O—, —NHC(S)—, and single bond;

A₆ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₈ alkyl, optionally substituted C₂₋₈ alkenyl, optionally substituted —O—C₁₋₆ alkyl, optionally substituted —O C₂₋₆ alkenyl, —OSO₂CF₃, and any natural or non-natural amino acid side chain;

A₇ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₈ alkyl, —S—, S(═O)—, —SO₂—, —O—, —C(═S)—, —C(═O)—, —NR—, —CH═CH—, —OC(O)NH—, —NHC(O)NH—, —NHC(O)O—, —NHC(O)—, —NHC(S)NH—, —NHC(S)O—, —NHC(S)—, and single bond;

when A₅ and A₇ are single bond, A₆ is directly attached to the carbon to which R⁸ is attached;

A₈ is a ring member of A₁ and is selected from the group consisting of C and N;

R is independently selected from —H, halo, optionally substituted C₁₋₄ alkyl, optionally substituted C₁₋₈ alkoxyalkyl, optionally substituted 2- to 5-membered polyethylene glycol, optionally substituted C₃₋₇ carbocyclyl, optionally substituted 5-10 membered heterocyclyl, optionally substituted C₆₋₁₀ aryl, optionally substituted C₆₋₁₀ aryl(C₁-C₆)alkyl, and optionally substituted 5-10 membered heteroaryl;

R² is independently selected from —H, optionally substituted C₁₋₄ alkyl, optionally substituted C₁₋₈ alkoxyalkyl, optionally substituted 2- to 5-membered polyethylene glycol, optionally substituted C₃₋₇ carbocyclyl, optionally substituted 5-10 membered heterocyclyl, optionally substituted C₆₋₁₀ aryl, and optionally substituted C₆₋₁₀ aryl(C₁-C₆)alkyl;

R⁶ is independently selected from —H and optionally substituted C₁₋₄ alkyl; and each n is independently selected to be an integer from 0 to 3.

In some embodiments, the calpain inhibitor may be a compound having the structure of Formula VI,

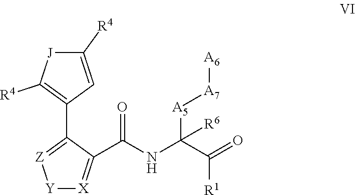

or a pharmaceutically acceptable salt thereof, wherein:

A₅ is selected from the group consisting of optionally substituted 3-10 membered heterocyclyl, optionally substituted C₆₋₁₀ aryl, optionally substituted 5-10 membered heteroaryl, optionally substituted C₃₋₁₀ carbocyclyl, optionally substituted C₁₋₈ alkyl, —S—, —S(═O)—, —SO₂—, —O—, —C(═S)—, —C(═O)—, —NR—, —CH═CH—, —OC(O)NH—, —NHC(O)NH—, —NHC(O)O—, —NHC(O)—, —NHC(S)NH—, —NHC(S)O—, —NHC(S)—, and single bond;

A₆ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₈ alkyl, optionally substituted C₂₋₈ alkenyl, optionally substituted —O—C₁₋₆ alkyl, optionally substituted —O C₂₋₆ alkenyl, —OSO₂CF₃, and any natural or non-natural amino acid side chain;

A₇ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₈ alkyl, —S—, S(═O)—, —SO₂—, —O—, —C(═S)—, —C(═O)—, —NR—, —CH═CH—, —OC(O)NH—, —NHC(O)NH—, —NHC(O)O—, —NHC(O)—, —NHC(S)NH—, —NHC(S)O—, —NHC(S)—, and single bond;

when A₅ and A₇ are single bond, A₆ is directly attached to the carbon to which R⁶ is attached;

Y is selected from the group consisting of NR⁵, and S;

X and Z are each independently selected from the group consisting of C(R⁴) and N;

J is selected from the group consisting of 0 and S;

each R⁴ is independently selected from the group consisting of —H, C₁₋₄ alkyl, C₁₋₄ haloalkyl, C₃₋₇ carbocyclyl (optionally substituted with halo, C₁-C₆ alkyl, C₁-C₆ alkoxy, C₁-C₆ haloalkyl, and C₁-C₆ haloalkoxy), halo, hydroxy, and C₁-C₆ alkoxy; and

R⁵ is selected from the group consisting of —H, C₁₋₄ alkyl, C₁₋₄ haloalkyl, and C₃₋₇ carbocyclyl (optionally substituted with halo, C₁-C₆ alkyl, C₁-C₆ alkoxy, C₁-C₆ haloalkyl, and C₁-C₆ haloalkoxy);

R¹ is selected from the group consisting of H, —OH, —COOR², C₁₋₄ haloalkyl, —COOH, —CH₂NO₂, —C(═O)NOR, —NH₂, —CONR²R³, —CH(CH₃)═CH₂, —CH(CF₃)NR²R³, —C(F)═CHCH₂CH₃,

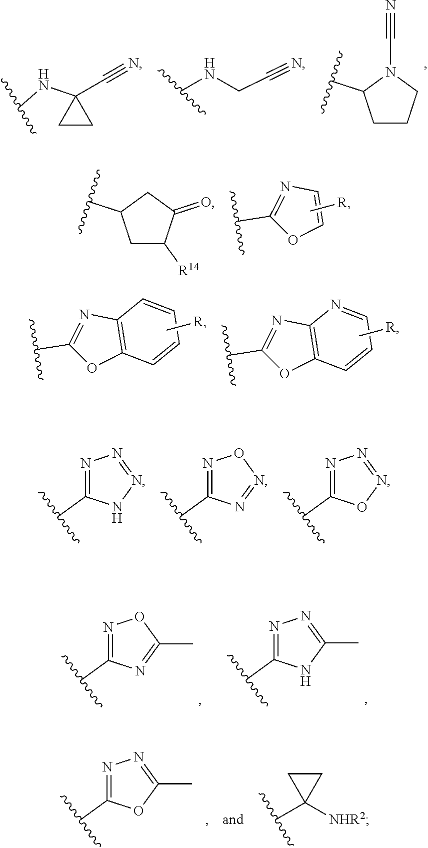

R¹⁴ is halo;

each R, R², and R³ are independently selected from —H, optionally substituted C₁₋₄ alkyl, optionally substituted C₁₋₈ alkoxyalkyl, optionally substituted 2- to 5-membered polyethylene glycol, optionally substituted C₃₋₇ carbocyclyl, optionally substituted 5-10 membered heterocyclyl, optionally substituted C₆₋₁₀ aryl, optionally substituted C₆₋₁₀ aryl(C₁-C₆)alkyl, and optionally substituted 5-10 membered heteroaryl;

R⁶ is independently selected from —H and optionally substituted C₁₋₄ alkyl; and each n is independently selected to be an integer from 0 to 3; and wherein the compound is not selected from the group consisting of

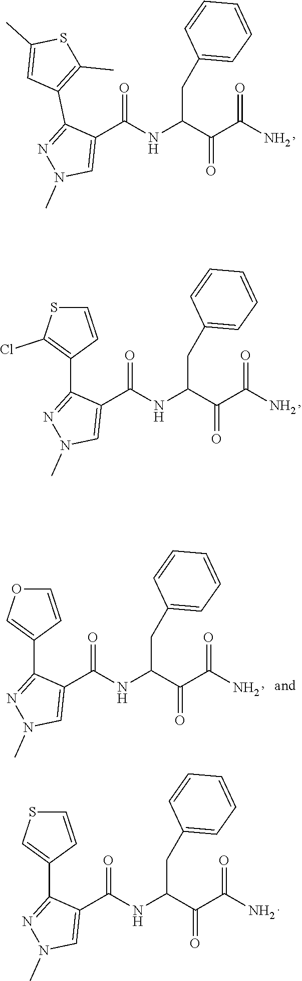 [SMILES: Cc1cc(-c2nn(C)cc2C(=O)NC(Cc2ccccc2)C(=O)C(N)=O)c(C)s1 ; Cn1cc(C(=O)NC(Cc2ccccc2)C(=O)C(N)=O)c(-c2ccsc2Cl)n1 ; Cn1cc(C(=O)NC(Cc2ccccc2)C(=O)C(N)=O)c(-c2ccoc2)n1 ; Cn1cc(C(=O)NC(Cc2ccccc2)C(=O)C(N)=O)c(-c2ccsc2)n1]

In some embodiments, the calpain inhibitor may be a compound having the structure of Formula VII,

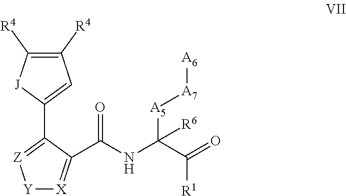

or a pharmaceutically acceptable salt thereof, wherein:

A₅ is selected from the group consisting of optionally substituted 3-10 membered heterocyclyl, optionally substituted C₆₋₁₀ aryl, optionally substituted 5-10 membered heteroaryl, optionally substituted C₃₋₁₀ carbocyclyl, optionally substituted C₁₋₈ alkyl, —S—, —S(═O)—, —SO₂—, —O—, —C(═S)—, —C(═O)—, —NR—, —CH═CH—, —OC(O)NH—, —NHC(O)NH—, —NHC(O)O—, —NHC(O)—, —NHC(S)NH—, —NHC(S)O—, —NHC(S)—, and single bond;

A₆ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₈ alkyl, optionally substituted C₂₋₈ alkenyl, optionally substituted —O—C₁₋₆ alkyl, optionally substituted —OC₂₋₆ alkenyl, —OSO₂CF₃, and any natural or non-natural amino acid side chain;

A₇ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₈ alkyl, —S—, S(═O)—, —SO₂—, —O—, —C(═S)—, —C(═O)—, —NR—, —CH═CH—, —OC(O)NH—, —NHC(O)NH—, —NHC(O)O—, —NHC(O)—, —NHC(S)NH—, —NHC(S)O—, —NHC(S)—, and single bond;

when A₅ and A₇ are single bond, A₆ is directly attached to the carbon to which R⁶ is attached;

Y is selected from the group consisting of NR⁵, and S;

X and Z are each independently selected from the group consisting of C(R⁴) and N;

J is selected from the group consisting of O and S;

each R⁴ is independently selected from the group consisting of —H, C₁₋₄ alkyl, C₁₋₄ haloalkyl, C₃₋₇ carbocyclyl (optionally substituted with halo, C₁-C₆ alkyl, C₁-C₆ alkoxy, C₁-C₆ haloalkyl, and C₁-C₆ haloalkoxy), halo, hydroxy, and C₁-C₆ alkoxy; and

R⁵ is selected from the group consisting of —H, C₁₋₄ alkyl, C₁₋₄ haloalkyl, and C₃₋₇ carbocyclyl (optionally substituted with halo, C₁-C₆ alkyl, C₁-C₆ alkoxy, C₁-C₆ haloalkyl, and C₁-C₆ haloalkoxy);

R¹ is selected from the group consisting of H, —OH, —COOR², C₁₋₄ haloalkyl, —COOH, —CH₂NO₂, —C(═O)NOR, —NH₂, —CONR²R³, —CH(CH₃)═CH₂, —CH(CF₃)NR²R³, —C(F)═CHCH₂CH₃,

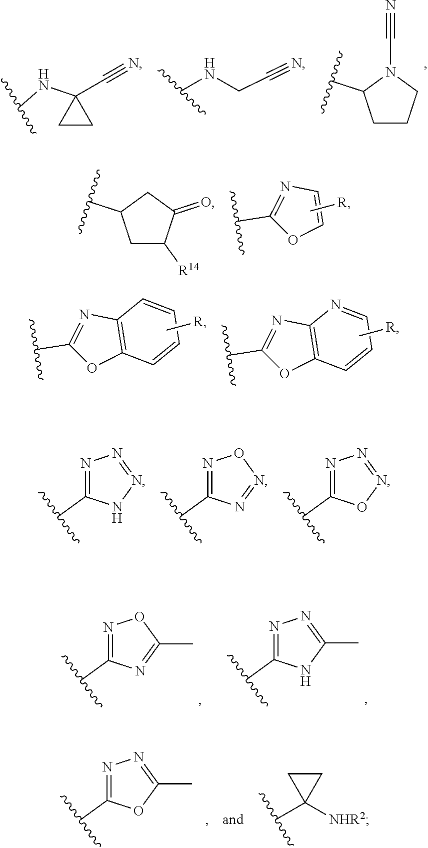

R¹⁴ is halo;

each R, R², and R³ are independently selected from —H, optionally substituted C₁₋₄ alkyl, optionally substituted C₁₋₈ alkoxyalkyl, optionally substituted 2- to 5-membered polyethylene glycol, optionally substituted C₃₋₇ carbocyclyl, optionally substituted 5-10 membered heterocyclyl, optionally substituted C₆₋₁₀ aryl, optionally substituted C₆₋₁₀ aryl(C₁-C₆)alkyl, and optionally substituted 5-10 membered heteroaryl;

R⁶ is independently selected from —H and optionally substituted C₁₋₄ alkyl; and

each n is independently selected to be an integer from 0 to 3; and wherein the compound is not selected from the group consisting of

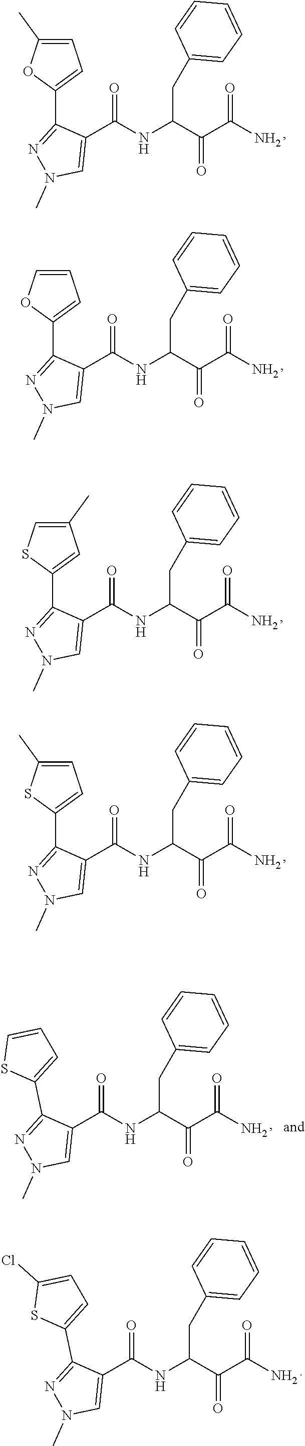

In some embodiments, the calpain inhibitor may be a compound having the structure of Formula VIII:

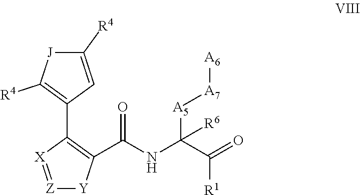

or a pharmaceutically acceptable salt thereof, wherein:

A₅ is selected from the group consisting of optionally substituted 3-10 membered heterocyclyl, optionally substituted C₆₋₁₀ aryl, optionally substituted 5-10 membered heteroaryl, optionally substituted C₃₋₁₀ carbocyclyl, optionally substituted C₁₋₈ alkyl, —S—, —S(═O)—, —SO₂—, —O—, —C(═S)—, —C(═O)—, —NR—, —CH═CH—, —OC(O)NH—, —NHC(O)NH—, —NHC(O)O—, —NHC(O)—, —NHC(S)NH—, —NHC(S)O—, —NHC(S)—, and single bond;

A₆ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₈ alkyl, optionally substituted C₂₋₈ alkenyl, optionally substituted —O—C₁₋₆ alkyl, optionally substituted —O C₂₋₆ alkenyl, —OSO₂CF₃, and any natural or non-natural amino acid side chain;

A₇ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₈ alkyl, —S—, S(═O)—, —SO₂—, —O—, —C(═S)—, —C(═O)—, —NR—, —CH═CH—, —OC(O)NH—, —NHC(O)NH—, —NHC(O)O—, —NHC(O)—, —NHC(S)NH—, —NHC(S)O—, —NHC(S)—, and single bond;

when A₅ and A₇ are single bond, A₆ is directly attached to the carbon to which R⁶ is attached;

Y is selected from the group consisting of NR⁵, O, S, and SO₂;

X and Z are each independently selected from the group consisting of C(R⁴) and N;

J is selected from the group consisting of 0 and S;

each R⁴ is independently selected from the group consisting of —H, C₁₋₄ alkyl, C₁₋₄ haloalkyl, C₃₋₇ carbocyclyl (optionally substituted with halo, C₁-C₆ alkyl, C₁-C₆ alkoxy, C₁-C₆ haloalkyl, and C₁-C₆ haloalkoxy), halo, hydroxy, and C₁-C₆ alkoxy; and

R⁵ is selected from the group consisting of —H, C₁₋₄ alkyl, C₁₋₄ haloalkyl, and C₃₋₇ carbocyclyl (optionally substituted with halo, C₁-C₆ alkyl, C₁-C₆ alkoxy, C₁-C₆ haloalkyl, and C₁-C₆ haloalkoxy);

R¹ is selected from the group consisting of H, —OH, —COOR², C₁₋₄ haloalkyl, —COOH, —CH₂NO₂, —C(═O)NOR, —NH₂, —CONR²R³, —CH(CH₃)═CH₂, —CH(CF₃)NR²R³, —C(F)═CHCH₂CH₃,

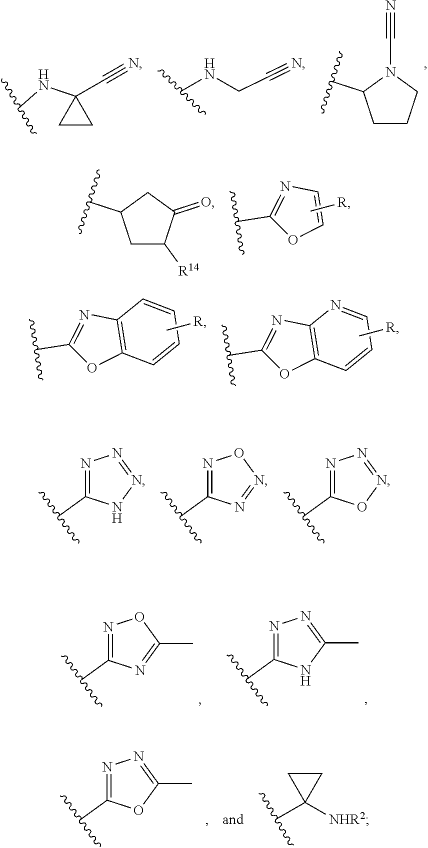

R¹⁴ is halo;

each R, R², and R³ are independently selected from —H, optionally substituted C₁₋₄ alkyl, optionally substituted C₁₋₈ alkoxyalkyl, optionally substituted 2- to 5-membered polyethylene glycol, optionally substituted C₃₋₇ carbocyclyl, optionally substituted 5-10 membered heterocyclyl, optionally substituted C₆₋₁₀ aryl, optionally substituted C₆₋₁₀ aryl(C₁-C₆)alkyl, and optionally substituted 5-10 membered heteroaryl;

R⁶ is independently selected from —H and optionally substituted C₁₋₄ alkyl; and

each n is independently selected to be an integer from 0 to 3.

In some embodiments, the calpain inhibitor may be a compound having the structure of Formula IX:

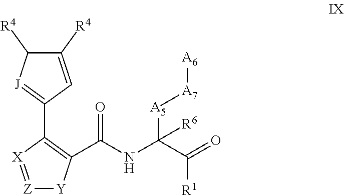

or a pharmaceutically acceptable salt thereof, wherein:

A₅ is selected from the group consisting of optionally substituted 3-10 membered heterocyclyl, optionally substituted C₆₋₁₀ aryl, optionally substituted 5-10 membered heteroaryl, optionally substituted C₃₋₁₀ carbocyclyl, optionally substituted C₁₋₈ alkyl, —S—, —S(═O)—, —SO₂—, —O—, —C(═S)—, —C(═O)—, —NR—, —CH═CH—, —OC(O)NH—, —NHC(O)NH—, —NHC(O)O—, —NHC(O)—, —NHC(S)NH—, —NHC(S)O—, —NHC(S)—, and single bond;

A₆ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₈ alkyl, optionally substituted C₂₋₈ alkenyl, optionally substituted —O—C₁₋₆ alkyl, optionally substituted —O C₂₋₆ alkenyl, —OSO₂CF₃, and any natural or non-natural amino acid side chain;

A₇ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₈ alkyl, —S—, S(═O)—, —SO₂—, —O—, —C(═S)—, —C(═O)—, —NR—, —CH═CH—, —OC(O)NH—, —NHC(O)NH—, —NHC(O)O—, —NHC(O)—, —NHC(S)NH—, —NHC(S)O—, —NHC(S)—, and single bond;

when A₅ and A₇ are single bond, A₆ is directly attached to the carbon to which R⁶ is attached;

Y is selected from the group consisting of NR⁵, O, S, and SO₂;

X and Z are each independently selected from the group consisting of C(R⁴) and N;

J is selected from the group consisting of 0 and S;

each R⁴ is independently selected from the group consisting of —H, C₁₋₄ alkyl, C₁₋₄ haloalkyl, C₃₋₇ carbocyclyl (optionally substituted with halo, C₁-C₆ alkyl, C₁-C₆ alkoxy, C₁-C₆ haloalkyl, and C₁-C₆ haloalkoxy), halo, hydroxy, and C₁-C₆ alkoxy; and

R⁵ is selected from the group consisting of —H, C₁₋₄ alkyl, C₁₋₄ haloalkyl, and C₃₋₇ carbocyclyl (optionally substituted with halo, C₁-C₆ alkyl, C₁-C₆ alkoxy, C₁-C₆ haloalkyl, and C₁-C₆ haloalkoxy);

R¹ is selected from the group consisting of H, —OH, —COOR², C₁₋₄ haloalkyl, —COOH, —CH₂NO₂, —C(═O)NOR, —NH₂, —CONR²R³, —CH(CH₃)═CH₂, —CH(CF₃)NR²R³, —C(F)═CHCH₂CH₃,

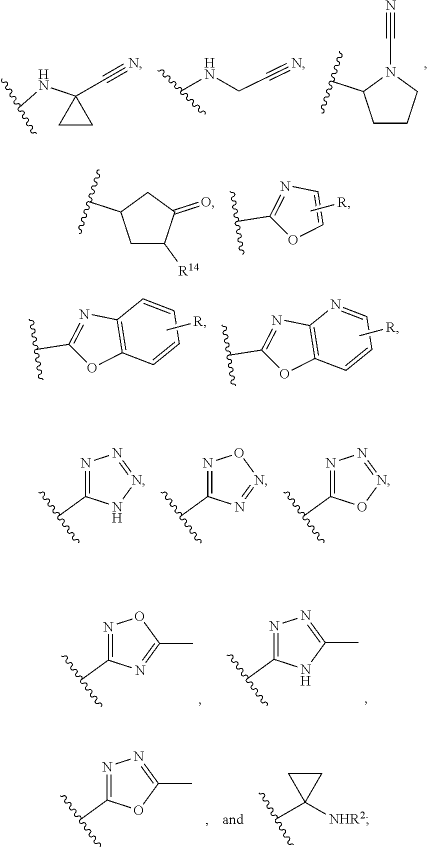

R¹⁴ is halo;

each R, R², and R³ are independently selected from —H, optionally substituted C₁₋₄ alkyl, optionally substituted C₁₋₈ alkoxyalkyl, optionally substituted 2- to 5-membered polyethylene glycol, optionally substituted C₃₋₇ carbocyclyl, optionally substituted 5-10 membered heterocyclyl, optionally substituted C₆₋₁₀ aryl, optionally substituted C₆₋₁₀ aryl(C₁-C₆)alkyl, and optionally substituted 5-10 membered heteroaryl;

R⁶ is independently selected from —H and optionally substituted C₁₋₄ alkyl; and each n is independently selected to be an integer from 0 to 3.

In some embodiments, the calpain inhibitor can be selected from the group consisting of the compounds listed in Table 4 below, or pharmaceutically acceptable salts thereof.

TABLE 4

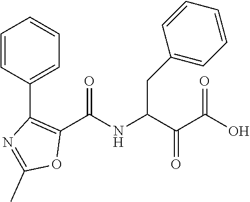 [SMILES: Cc1nc(-c2ccccc2)c(C(=O)NC(Cc2ccccc2)C(=O)C(=O)O)o1]

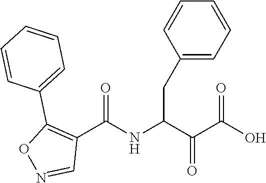 [SMILES: O=C(O)C(=O)C(Cc1ccccc1)NC(=O)c1cnoc1-c1ccccc1]

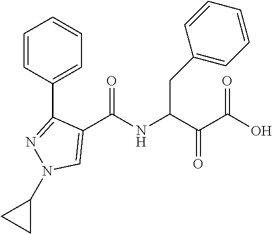 [SMILES: O=C(O)C(=O)C(Cc1ccccc1)NC(=O)c1cn(C2CC2)nc1-c1ccccc1]

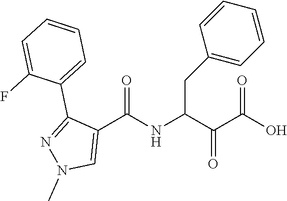 [SMILES: Cn1cc(C(=O)NC(Cc2ccccc2)C(=O)C(=O)O)c(-c2ccccc2F)n1]

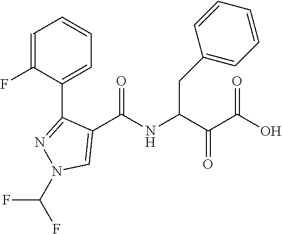 [SMILES: O=C(O)C(=O)C(Cc1ccccc1)NC(=O)c1cn(C(F)F)nc1-c1ccccc1F]

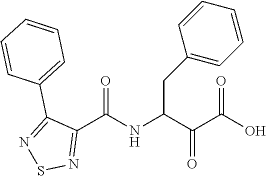 [SMILES: O=C(O)C(=O)C(Cc1ccccc1)NC(=O)c1nsnc1-c1ccccc1]

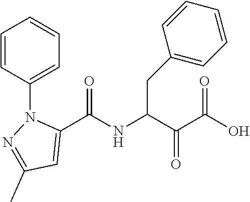 [SMILES: Cc1cc(C(=O)NC(Cc2ccccc2)C(=O)C(=O)O)n(-c2ccccc2)n1]

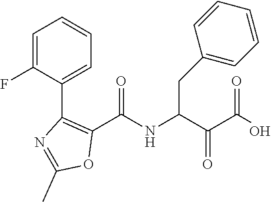 [SMILES: Cc1nc(-c2ccccc2F)c(C(=O)NC(Cc2ccccc2)C(=O)C(=O)O)o1]

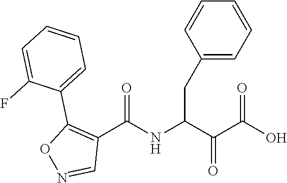 [SMILES: O=C(O)C(=O)C(Cc1ccccc1)NC(=O)c1cnoc1-c1ccccc1F]

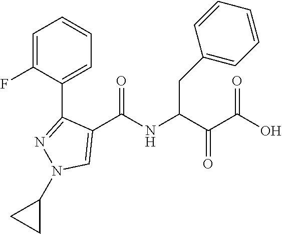 [SMILES: O=C(O)C(=O)C(Cc1ccccc1)NC(=O)c1cn(C2CC2)nc1-c1ccccc1F]

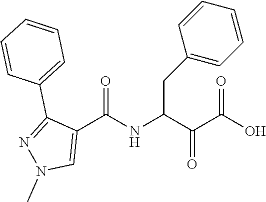 [SMILES: Cn1cc(C(=O)NC(Cc2ccccc2)C(=O)C(=O)O)c(-c2ccccc2)n1]

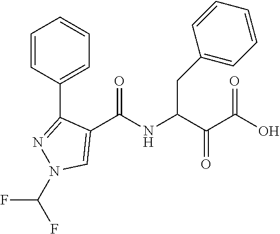 [SMILES: O=C(O)C(=O)C(Cc1ccccc1)NC(=O)c1cn(C(F)F)nc1-c1ccccc1]

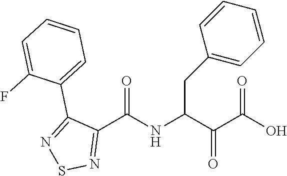 [SMILES: O=C(O)C(=O)C(Cc1ccccc1)NC(=O)c1nsnc1-c1ccccc1F]

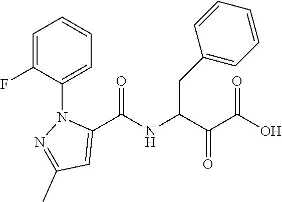 [SMILES: Cc1cc(C(=O)NC(Cc2ccccc2)C(=O)C(=O)O)n(-c2ccccc2F)n1]

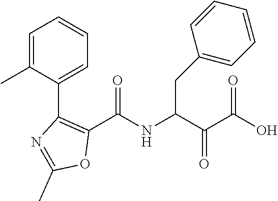 [SMILES: Cc1nc(-c2ccccc2C)c(C(=O)NC(Cc2ccccc2)C(=O)C(=O)O)o1]

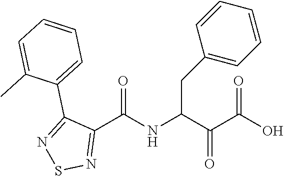 [SMILES: Cc1ccccc1-c1nsnc1C(=O)NC(Cc1ccccc1)C(=O)C(=O)O]

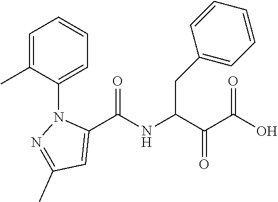 [SMILES: Cc1cc(C(=O)NC(Cc2ccccc2)C(=O)C(=O)O)n(-c2ccccc2C)n1]

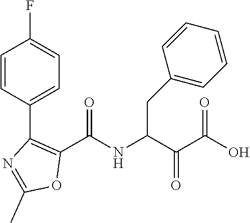 [SMILES: Cc1nc(-c2ccc(F)cc2)c(C(=O)NC(Cc2ccccc2)C(=O)C(=O)O)o1]

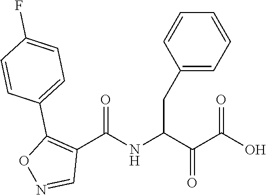 [SMILES: O=C(O)C(=O)C(Cc1ccccc1)NC(=O)c1cnoc1-c1ccc(F)cc1]

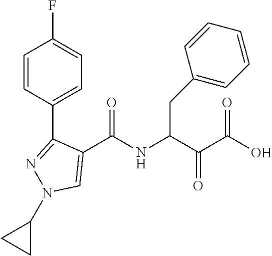 [SMILES: O=C(O)C(=O)C(Cc1ccccc1)NC(=O)c1cn(C2CC2)nc1-c1ccc(F)cc1]

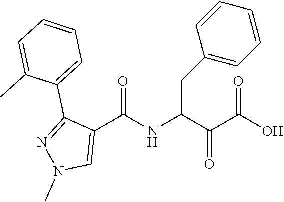 [SMILES: Cc1ccccc1-c1nn(C)cc1C(=O)NC(Cc1ccccc1)C(=O)C(=O)O]

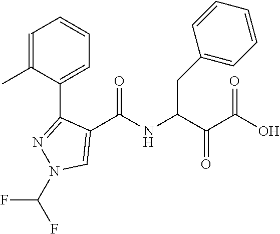 [SMILES: Cc1ccccc1-c1nn(C(F)F)cc1C(=O)NC(Cc1ccccc1)C(=O)C(=O)O]

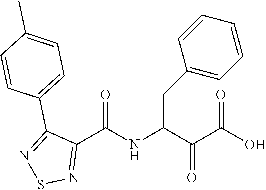 [SMILES: Cc1ccc(-c2nsnc2C(=O)NC(Cc2ccccc2)C(=O)C(=O)O)cc1]

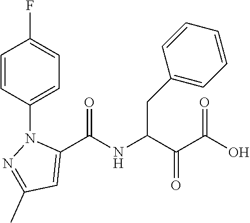 [SMILES: Cc1cc(C(=O)NC(Cc2ccccc2)C(=O)C(=O)O)n(-c2ccc(F)cc2)n1]

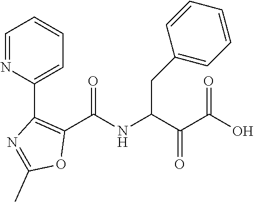 [SMILES: Cc1nc(-c2ccccn2)c(C(=O)NC(Cc2ccccc2)C(=O)C(=O)O)o1]

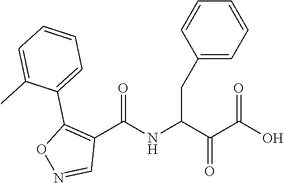 [SMILES: Cc1ccccc1-c1oncc1C(=O)NC(Cc1ccccc1)C(=O)C(=O)O]

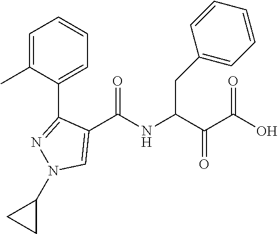 [SMILES: Cc1ccccc1-c1nn(C2CC2)cc1C(=O)NC(Cc1ccccc1)C(=O)C(=O)O]

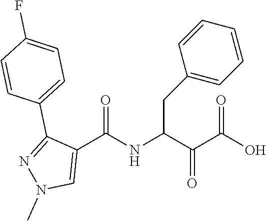 [SMILES: Cn1cc(C(=O)NC(Cc2ccccc2)C(=O)C(=O)O)c(-c2ccc(F)cc2)n1]

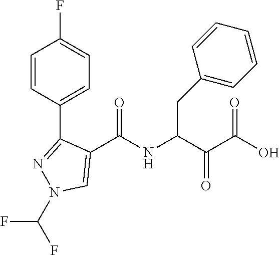 [SMILES: O=C(O)C(=O)C(Cc1ccccc1)NC(=O)c1cn(C(F)F)nc1-c1ccc(F)cc1]

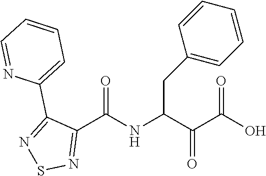 [SMILES: O=C(O)C(=O)C(Cc1ccccc1)NC(=O)c1nsnc1-c1ccccn1]

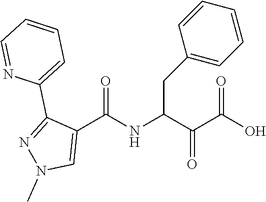 [SMILES: Cn1cc(C(=O)NC(Cc2ccccc2)C(=O)C(=O)O)c(-c2ccccn2)n1]

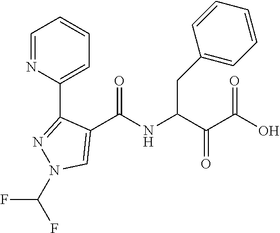 [SMILES: O=C(O)C(=O)C(Cc1ccccc1)NC(=O)c1cn(C(F)F)nc1-c1ccccn1]

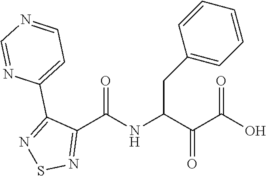 [SMILES: O=C(O)C(=O)C(Cc1ccccc1)NC(=O)c1nsnc1-c1ccncn1]

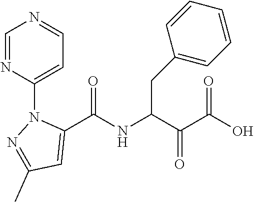 [SMILES: Cc1cc(C(=O)NC(Cc2ccccc2)C(=O)C(=O)O)n(-c2ccncn2)n1]

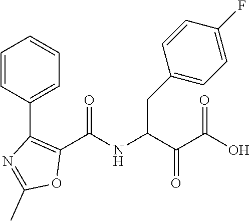 [SMILES: Cc1nc(-c2ccccc2)c(C(=O)NC(Cc2ccc(F)cc2)C(=O)C(=O)O)o1]

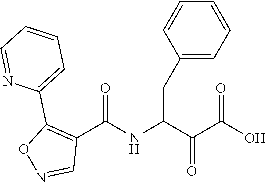 [SMILES: O=C(O)C(=O)C(Cc1ccccc1)NC(=O)c1cnoc1-c1ccccn1]

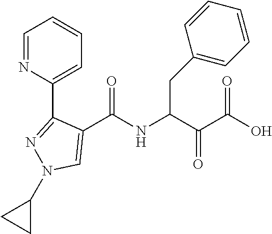 [SMILES: O=C(O)C(=O)C(Cc1ccccc1)NC(=O)c1cn(C2CC2)nc1-c1ccccn1]

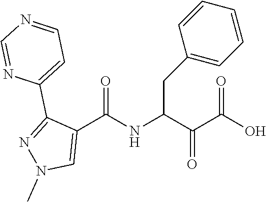 [SMILES: Cn1cc(C(=O)NC(Cc2ccccc2)C(=O)C(=O)O)c(-c2ccncn2)n1]

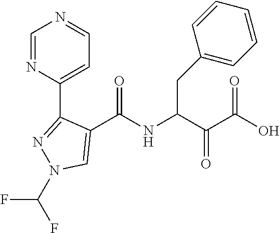 [SMILES: O=C(O)C(=O)C(Cc1ccccc1)NC(=O)c1cn(C(F)F)nc1-c1ccncn1]

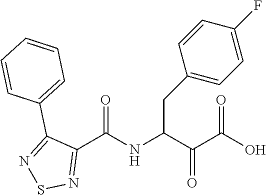 [SMILES: O=C(O)C(=O)C(Cc1ccc(F)cc1)NC(=O)c1nsnc1-c1ccccc1]

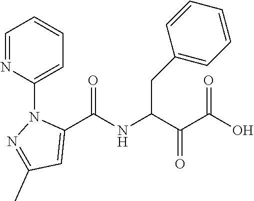 [SMILES: Cc1cc(C(=O)NC(Cc2ccccc2)C(=O)C(=O)O)n(-c2ccccn2)n1]

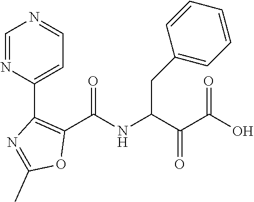 [SMILES: Cc1nc(-c2ccncn2)c(C(=O)NC(Cc2ccccc2)C(=O)C(=O)O)o1]

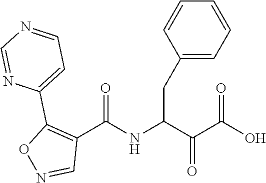 [SMILES: O=C(O)C(=O)C(Cc1ccccc1)NC(=O)c1cnoc1-c1ccncn1]

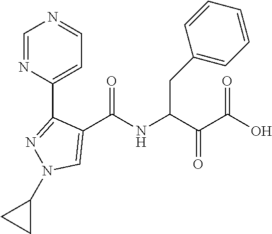 [SMILES: O=C(O)C(=O)C(Cc1ccccc1)NC(=O)c1cn(C2CC2)nc1-c1ccncn1]

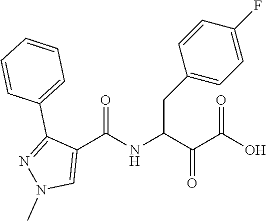 [SMILES: Cn1cc(C(=O)NC(Cc2ccc(F)cc2)C(=O)C(=O)O)c(-c2ccccc2)n1]

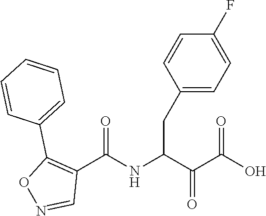 [SMILES: O=C(O)C(=O)C(Cc1ccc(F)cc1)NC(=O)c1cnoc1-c1ccccc1]

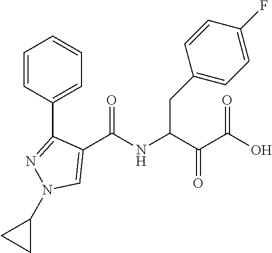 [SMILES: O=C(O)C(=O)C(Cc1ccc(F)cc1)NC(=O)c1cn(C2CC2)nc1-c1ccccc1]

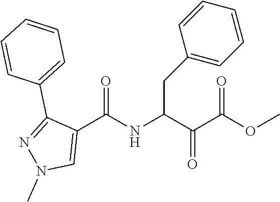 [SMILES: COC(=O)C(=O)C(Cc1ccccc1)NC(=O)c1cn(C)nc1-c1ccccc1]

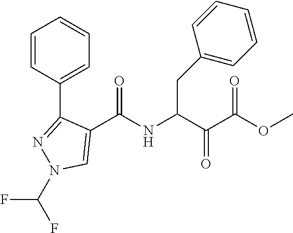 [SMILES: COC(=O)C(=O)C(Cc1ccccc1)NC(=O)c1cn(C(F)F)nc1-c1ccccc1]

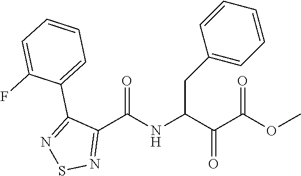 [SMILES: COC(=O)C(=O)C(Cc1ccccc1)NC(=O)c1nsnc1-c1ccccc1F]

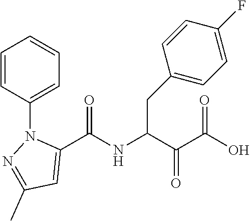 [SMILES: Cc1cc(C(=O)NC(Cc2ccc(F)cc2)C(=O)C(=O)O)n(-c2ccccc2)n1]

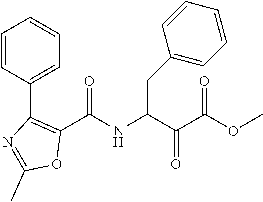 [SMILES: COC(=O)C(=O)C(Cc1ccccc1)NC(=O)c1oc(C)nc1-c1ccccc1]

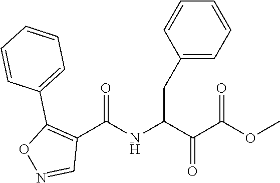 [SMILES: COC(=O)C(=O)C(Cc1ccccc1)NC(=O)c1cnoc1-c1ccccc1]

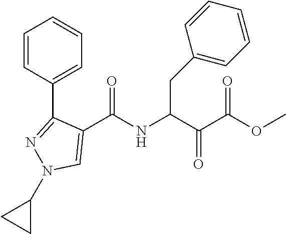 [SMILES: COC(=O)C(=O)C(Cc1ccccc1)NC(=O)c1cn(C2CC2)nc1-c1ccccc1]

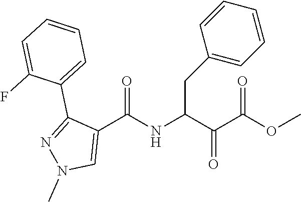 [SMILES: COC(=O)C(=O)C(Cc1ccccc1)NC(=O)c1cn(C)nc1-c1ccccc1F]

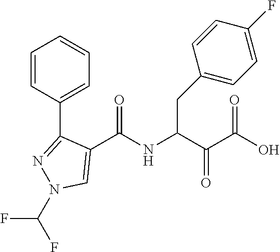 [SMILES: O=C(O)C(=O)C(Cc1ccc(F)cc1)NC(=O)c1cn(C(F)F)nc1-c1ccccc1]

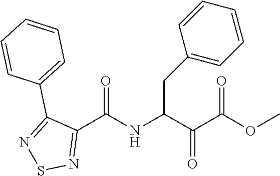 [SMILES: COC(=O)C(=O)C(Cc1ccccc1)NC(=O)c1nsnc1-c1ccccc1]

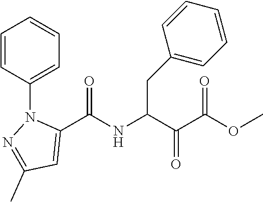 [SMILES: COC(=O)C(=O)C(Cc1ccccc1)NC(=O)c1cc(C)nn1-c1ccccc1]

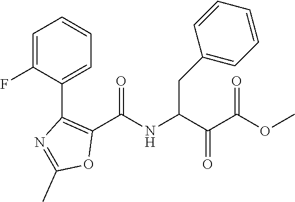 [SMILES: COC(=O)C(=O)C(Cc1ccccc1)NC(=O)c1oc(C)nc1-c1ccccc1F]

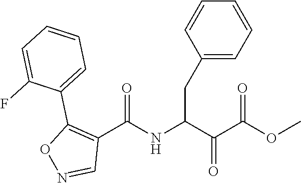 [SMILES: COC(=O)C(=O)C(Cc1ccccc1)NC(=O)c1cnoc1-c1ccccc1F]

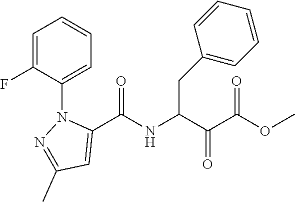 [SMILES: COC(=O)C(=O)C(Cc1ccccc1)NC(=O)c1cc(C)nn1-c1ccccc1F]

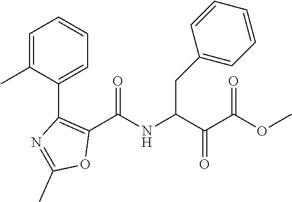

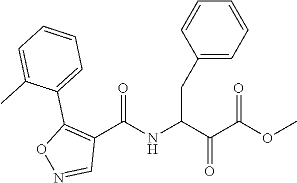

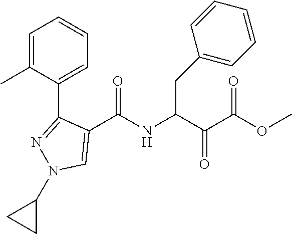

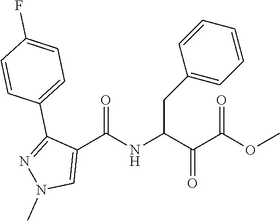 [SMILES: COC(=O)C(=O)C(Cc1ccccc1)NC(=O)c1cn(C)nc1-c1ccc(F)cc1]

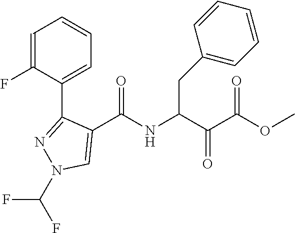 [SMILES: COC(=O)C(=O)C(Cc1ccccc1)NC(=O)c1cn(C(F)F)nc1-c1ccccc1F]

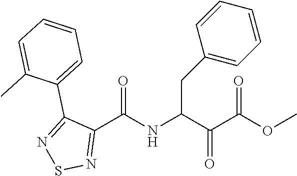

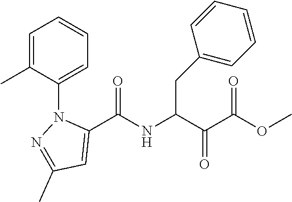

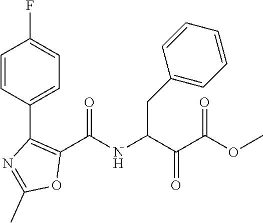 [SMILES: COC(=O)C(=O)C(Cc1ccccc1)NC(=O)c1oc(C)nc1-c1ccc(F)cc1]

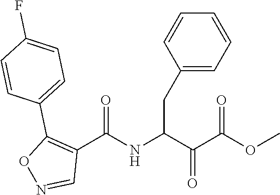 [SMILES: COC(=O)C(=O)C(Cc1ccccc1)NC(=O)c1cnoc1-c1ccc(F)cc1]

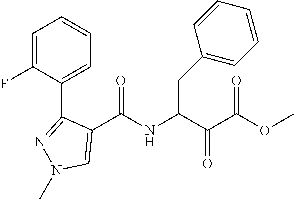 [SMILES: COC(=O)C(=O)C(Cc1ccccc1)NC(=O)c1cn(C)nc1-c1ccccc1F]

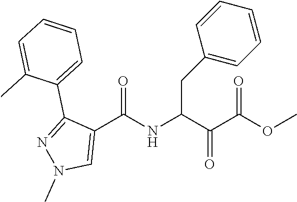

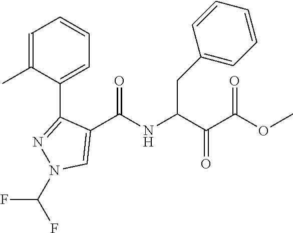

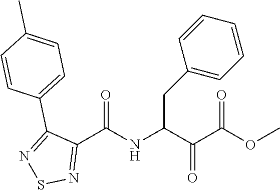 [SMILES: COC(=O)C(=O)C(Cc1ccccc1)NC(=O)c1nsnc1-c1ccc(C)cc1]

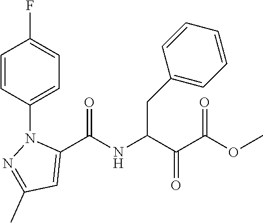 [SMILES: COC(=O)C(=O)C(Cc1ccccc1)NC(=O)c1cc(C)nn1-c1ccc(F)cc1]

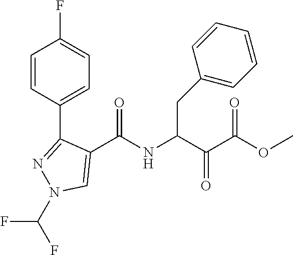 [SMILES: COC(=O)C(=O)C(Cc1ccccc1)NC(=O)c1cn(C(F)F)nc1-c1ccc(F)cc1]

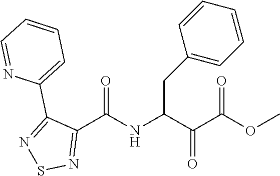 [SMILES: COC(=O)C(=O)C(Cc1ccccc1)NC(=O)c1nsnc1-c1ccccn1]

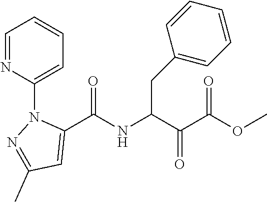 [SMILES: COC(=O)C(=O)C(Cc1ccccc1)NC(=O)c1cc(C)nn1-c1ccccn1]

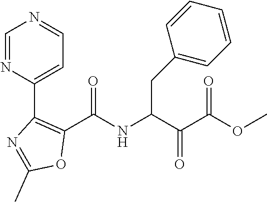 [SMILES: COC(=O)C(=O)C(Cc1ccccc1)NC(=O)c1oc(C)nc1-c1ccncn1]

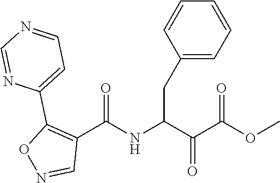 [SMILES: COC(=O)C(=O)C(Cc1ccccc1)NC(=O)c1cnoc1-c1ccncn1]

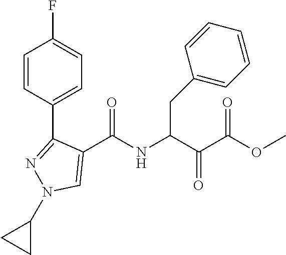 [SMILES: COC(=O)C(=O)C(Cc1ccccc1)NC(=O)c1cn(C2CC2)nc1-c1ccc(F)cc1]

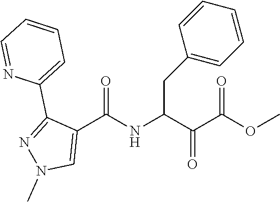 [SMILES: COC(=O)C(=O)C(Cc1ccccc1)NC(=O)c1cn(C)nc1-c1ccccn1]

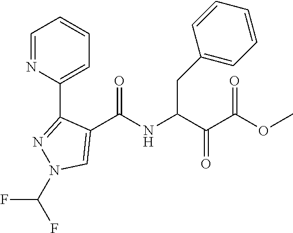 [SMILES: COC(=O)C(=O)C(Cc1ccccc1)NC(=O)c1cn(C(F)F)nc1-c1ccccn1]

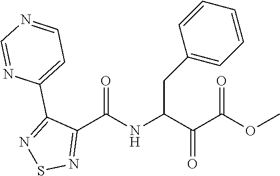 [SMILES: COC(=O)C(=O)C(Cc1ccccc1)NC(=O)c1nsnc1-c1ccncn1]

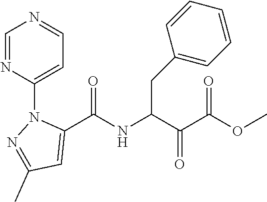 [SMILES: COC(=O)C(=O)C(Cc1ccccc1)NC(=O)c1cc(C)nn1-c1ccncn1]

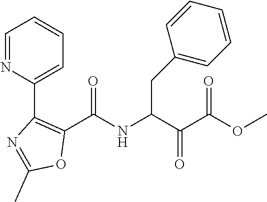 [SMILES: COC(=O)C(=O)C(Cc1ccccc1)NC(=O)c1oc(C)nc1-c1ccccn1]

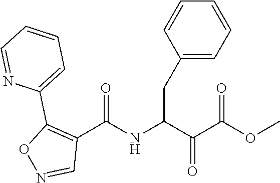 [SMILES: COC(=O)C(=O)C(Cc1ccccc1)NC(=O)c1cnoc1-c1ccccn1]

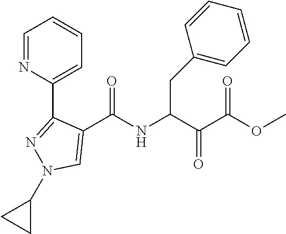 [SMILES: COC(=O)C(=O)C(Cc1ccccc1)NC(=O)c1cn(C2CC2)nc1-c1ccccn1]

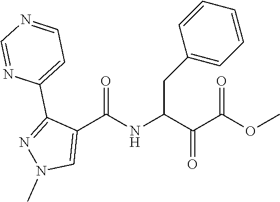 [SMILES: COC(=O)C(=O)C(Cc1ccccc1)NC(=O)c1cn(C)nc1-c1ccncn1]

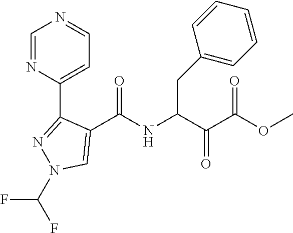 [SMILES: COC(=O)C(=O)C(Cc1ccccc1)NC(=O)c1cn(C(F)F)nc1-c1ccncn1]

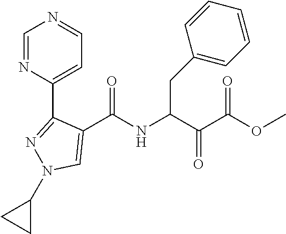 [SMILES: COC(=O)C(=O)C(Cc1ccccc1)NC(=O)c1cn(C2CC2)nc1-c1ccncn1]

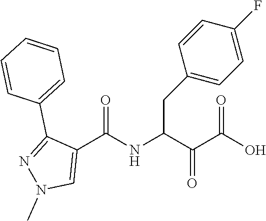 [SMILES: Cn1cc(C(=O)NC(Cc2ccc(F)cc2)C(=O)C(=O)O)c(-c2ccccc2)n1]

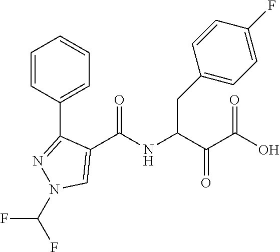 [SMILES: O=C(O)C(=O)C(Cc1ccc(F)cc1)NC(=O)c1cn(C(F)F)nc1-c1ccccc1]

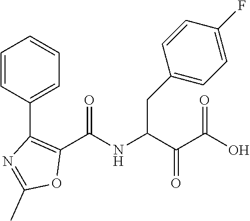 [SMILES: Cc1nc(-c2ccccc2)c(C(=O)NC(Cc2ccc(F)cc2)C(=O)C(=O)O)o1]

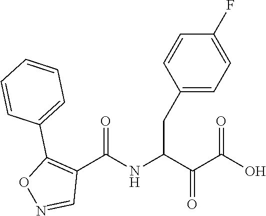 [SMILES: O=C(O)C(=O)C(Cc1ccc(F)cc1)NC(=O)c1cnoc1-c1ccccc1]

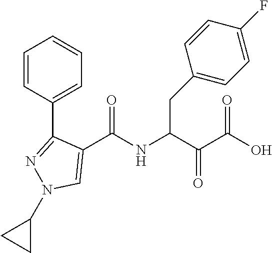 [SMILES: O=C(O)C(=O)C(Cc1ccc(F)cc1)NC(=O)c1cn(C2CC2)nc1-c1ccccc1]

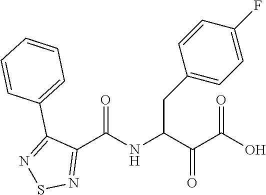 [SMILES: O=C(O)C(=O)C(Cc1ccc(F)cc1)NC(=O)c1nsnc1-c1ccccc1]

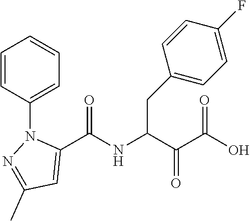 [SMILES: Cc1cc(C(=O)NC(Cc2ccc(F)cc2)C(=O)C(=O)O)n(-c2ccccc2)n1]

The compounds of Formula V-IX and Table 4 may be prepared according to the methods described in the Examples provided herein.

In some embodiments provided herein is a method of treating a disease or disorder selected from the group consisting of primary sclerosing cholangitis, primary biliary cholangitis, non-alcoholic fatty liver disease, non-alcoholic steatohepatitis, and liver cirrhosis; the method comprising administering one or more calpain inhibitors to a subject in need thereof.

In some embodiments, the liver cirrhosis is caused by one or more of the conditions selected from the group consisting of alcoholic liver disease, alpha-1 antitrypsin deficiency, autoimmune hepatitis, celiac disease, chronic viral hepatitis, hemochromatosis, idiopathic portal fibrosis, and Wilson disease.

In some embodiments, the one or more calpain inhibitors may be a compound disclosed herein. In some embodiments, the one or more calpain inhibitiors may be a compound of any one of Formula I, II, III, IV, V, VI, VII, VIII, or IX. In some embodiments, the calpain inhibitor may be a compound of Formula I. In some embodiments, the calpain inhibitor may be a compound of Formula II. In some embodiments, the calpain inhibitor may be a compound of Formula III. In some embodiments, the calpain inhibitor may be a compound of Formula IV. In some embodiments, the calpain inhibitor may be a compound of Formula V. In some embodiments, the calpain inhibitor may be a compound of Formula VI. In some embodiments, the calpain inhibitor may be a compound of Formula VII. In some embodiments, the calpain inhibitor may be a compound of Formula VIII. In some embodiments, the calpain inhibitor may be a compound of Formula IX.

In some embodiments, the calpain inhibitor may be a compound listed in any one of Table 1a, 1b, 2, 3, and 4. In some embodiments, the calpain inhibitor may be a compound listed in Table 1a or 1b. In some embodiments, the calpain inhibitor may be a compound listed in Table 2. In some embodiments, the calpain inhibitor may be a compound listed in Table 3. In some embodiments, the calpain inhibitor may be a compound listed in Table 4.

In some embodiments, the calpain inhibitor may be selected from the group consisting of:

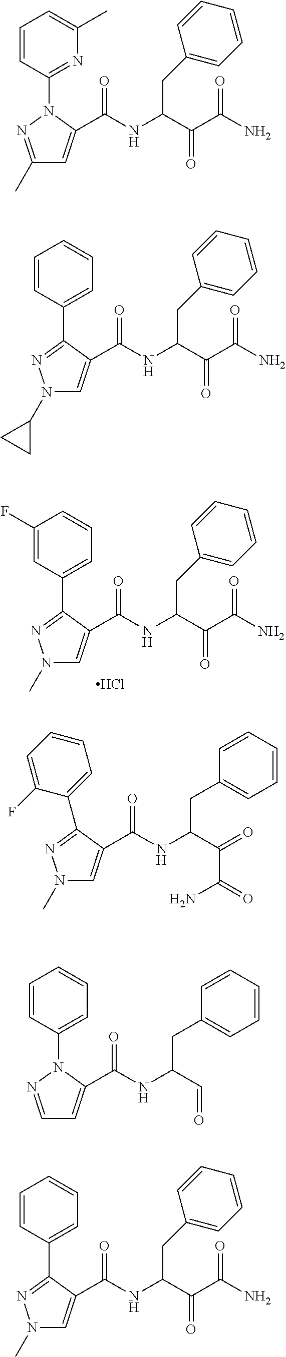

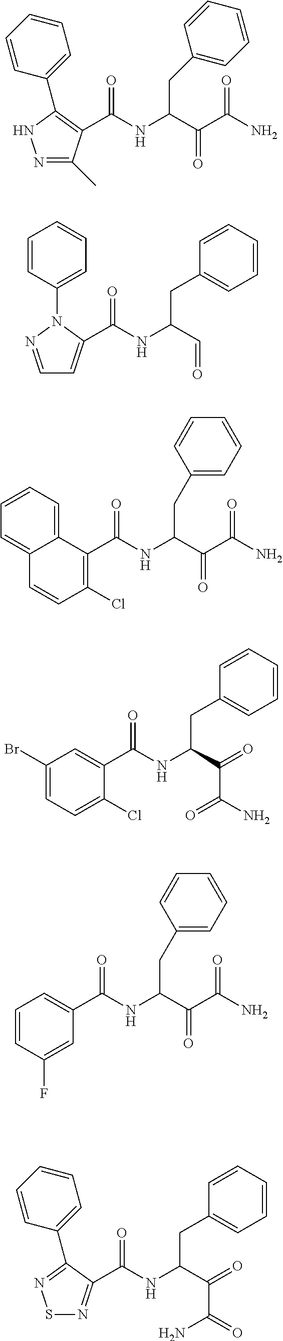

or pharmaceutically acceptable salts thereof.

In some embodiments, the calpain inhibitor may be selected from the group consisting of:

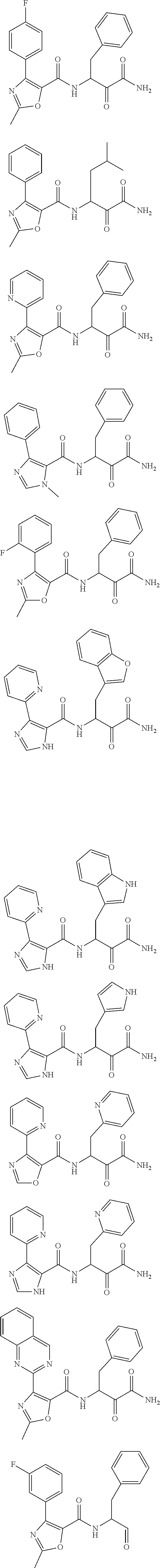

or pharmaceutically acceptable salts thereof.

In some embodiments, the calpain inhibitor may be selected from the group consisting of:

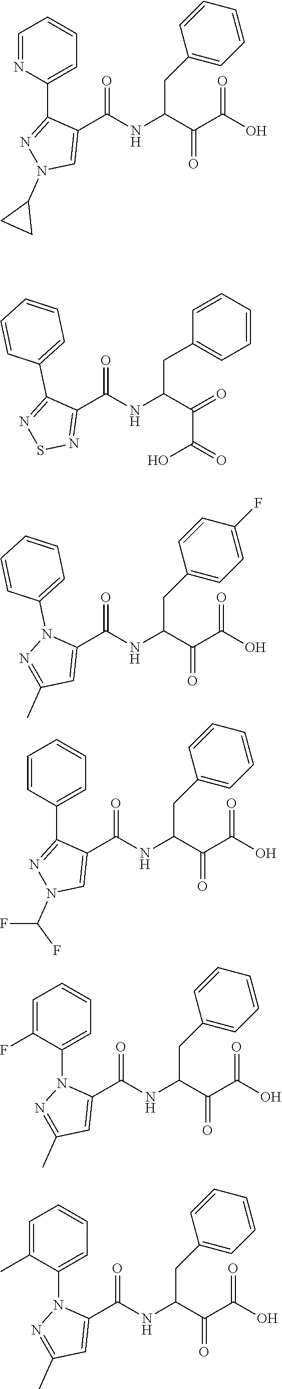

or pharmaceutically acceptable salts thereof.

In some embodiments, the one or more calpain inhibitors may be administered in combination with one or more additional agents selected from the group consisting of a VAP-1 inhibitor, an ASBT Inhibitor, a dual CCR2/5 antagonist, an anti-cholestatic bile acid, a FXR agonist, a FGFR1c/4 agonist, mesenchymal stem cell (MSC) cell therapy, a CCL24 Inhibitor, and a CCL11 inhibitor. In some embodiments, the one or more calpain inhibitors and the one or more additional aforementioned agents may be used to treat primary sclerosing cholangitis in a subject.

In some embodiments, the calpain inhibitor may be administered in combination with one or more additional agents selected from the group consisting of obeticholic acid, elafibranor, cenicriviroc, selonsertib, a niacin receptor agonist, a SGLT2 inhibitor, a VAP-1 inhibitor, a FGF21 mimetic, a adenosine A3 receptor agonist, a mTOT modulator, a FXR agonist, a galectin-3 inhibitor, an ABCA1 activator, a SCD1 inhibitor, an ACC inhibitor, a Type I NK T-cell inhibitor, a pan-PPAR agonist, a DGAT2 inhibitor, a PPARalpha agonist, a thyroid hormone R-b agonist, a 5-LO/LT inhibitor, a mineralocorticoid receptor antagonist, a FGF19 mimic, a caspase inhibitor, a GLP-1R agonist, a SIRT1/AMP agonist, an ACC inhibitor, a ketohexokinase inhibitor, a GLP-1R agonist, an ASBT inhibitor, a DGAT2/CYP2E1 inhibitor, a TLR4 antagonist, a thyroid hormone R-b agonist, a IFN-gamma receptor antagonism, a CB1 antagonist, a FGF21 ligand, a P2Y13 receptor agonist, a CCL24 inhibitor, a MCH receptor-1 antagonist, aPPARalpha, delta agonist, a DPP-4 inhibitor, aLXR antagonist, a GLP1R agonist, an eotaxin-1 inhibitor, a beta-klotho/FGFR1c agonist, a LOXL2 Inhibitor, an AMPK activator, a miR-103/107 inhibitor, an inflammasome inhibitor, a CD3 antagonist, and a cathepsin B inhibitor. In some embodiments, the one or more calpain inhibitors and the one or more additional aforementioned agents may be used to treat non-alcoholic steatohepatitis (NASH) in a subject.

The following examples are included for illustrative purposes. The examples should not, of course, be construed as specifically limiting the scope of the disclosure. Variations of these examples within the scope of the claims are within the purview of one skilled in the art and are considered to fall within the scope of the disclosure as described, and claimed herein. The reader will recognize that the skilled artisan, armed with the present disclosure, and skill in the art is able to prepare and use the subject matter described herein without exhaustive examples. The following examples will further describe the present disclosure, and are used for the purposes of illustration only, and should not be considered as limiting.

### EXAMPLES

It will be apparent to the skilled artisan that methods for preparing precursors and functionality related to the compounds claimed herein are generally described in the literature. In these reactions, it is also possible to make use of variants which are themselves known to those of ordinary skill in this art, but are not mentioned in greater detail. The skilled artisan given the literature and this disclosure is well equipped to prepare any of the compounds.

It is recognized that the skilled artisan in the art of organic chemistry can readily carry out manipulations without further direction, that is, it is well within the scope and practice of the skilled artisan to carry out these manipulations. These include reduction of carbonyl compounds to their corresponding alcohols, oxidations, acylations, aromatic substitutions, both electrophilic and nucleophilic, etherifications, esterification and saponification and the like. These manipulations are discussed in standard texts such as March Advanced Organic Chemistry (Wiley), Carey and Sundberg, Advanced Organic Chemistry (incorporated herein by reference in their entirety) and the like. All the intermediate compounds of the present invention were used without further purification unless otherwise specified.

The skilled artisan will readily appreciate that certain reactions are best carried out when other functionality is masked or protected in the molecule, thus avoiding any undesirable side reactions and/or increasing the yield of the reaction. Often the skilled artisan utilizes protecting groups to accomplish such increased yields or to avoid the undesired reactions. These reactions are found in the literature and are also well within the scope of the skilled artisan. Examples of many of these manipulations can be found for example in T. Greene and P. Wuts Protecting Groups in Organic Synthesis, 4th Ed., John Wiley & Sons (2007), incorporated herein by reference in its entirety.

The following example schemes are provided for the guidance of the reader, and represent preferred methods for making the compounds exemplified herein. These methods are not limiting, and it will be apparent that other routes may be employed to prepare these compounds. Such methods specifically include solid phase based chemistries, including combinatorial chemistry. The skilled artisan is thoroughly equipped to prepare these compounds by those methods given the literature and this disclosure. The compound numberings used in the synthetic schemes depicted below are meant for those specific schemes only, and should not be construed as or confused with same numberings in other sections of the application.

Trademarks used herein are examples only and reflect illustrative materials used at the time of the invention. The skilled artisan will recognize that variations in lot, manufacturing processes, and the like, are expected. Hence the examples, and the trademarks used in them are non-limiting, and they are not intended to be limiting, but are merely an illustration of how a skilled artisan may choose to perform one or more of the embodiments of the invention.

The following abbreviations have the indicated meanings:
DCM=dichloromethane
DIEA=N,N-Diisopropylethylamine
DIPEA=N,N-Diisopropylethylamine
DMF=N,N-dimethylformamide
DMP=Dess Martin Periodinane
DNs=dinitrosulfonyl
ESBL=extended-spectrum β-lactamase
EtOAc=ethyl acetate
EA=ethyl acetate
FCC=Flash Column Chromatography
FDPP=Pentaflurophenyl diphenylphosphinate
HATU=2-(7-aza-1H-benzotriazole-1-yl)-1,1,3,3-tetramethyluronium hexafluorophosphate
MeCN=acetonitrile
NMR=nuclear magnetic resonance
PE=Petroleum Ether
Prep=preparatory
Py=pyridine
Sat.=saturated aqueous
TBDMSCl=tert-butyldimethylsilyl chloride
TBS=tert-butyldimethylsilyl
TFA=trifluoroacetic acid
THF=tetrahydrofuran
TLC=thin layer chromatography

The example schemes shown below are provided for the guidance of the reader, and collectively represent an example method for making the compounds encompassed herein. Furthermore, other methods for preparing compounds described herein will be readily apparent to the person of ordinary skill in the art in light of the following reaction schemes and examples. Unless otherwise indicated, all variables are as defined above.

Example Sections I, II, and III have independently numbered Examples and compounds numbers. References to compound numbers or Example numbers found in any of Example Section I, II, and III refer to the compounds and Examples of that particular section.

### EXAMPLE SECTION I

### Example 1

### (S)—N-(4-amino-3,4-dioxo-1-phenylbutan-2-yl)-2,6-dichlorobenzamide (1)

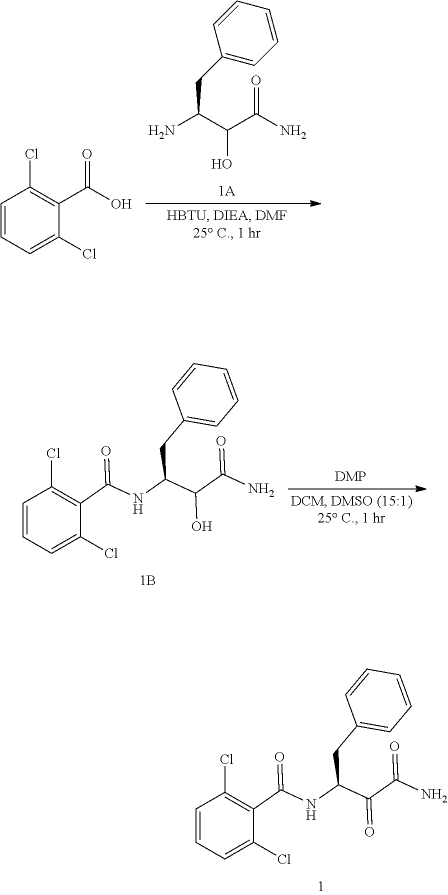 [SMILES: O=C(O)c1c(Cl)cccc1Cl ; NC(=O)C(O)[C@@H](N)Cc1ccccc1 ; NC(=O)C(O)[C@H](Cc1ccccc1)NC(=O)c1c(Cl)cccc1Cl ; NC(=O)C(=O)[C@H](Cc1ccccc1)NC(=O)c1c(Cl)cccc1Cl]

To a solution of compound 2,6-dichlorobenzoic acid (300 mg, 1.57 mmol) and compound 1A (366.1 mg, 1.59 mmol) in DMF (8 mL) was added HBTU (714.8 mg, 1.88 mmol). The mixture was stirred at 25° C. for 0.1 hour, and then DIEA (204.9 mg, 1.59 mmol) was added. The resultant mixture was stirred at 25° C. for 1 hour. The reaction mixture was diluted with EtOAc (100 mL), washed successively with 1N HCl (20 mL), sat. NaHCO₃ (50 mL×2), water (50 mL) and brine (50 mL), dried over anhydrous Na₂SO₄, filtered and concentrated under reduced pressure to give a pink solid, which was purified by triturating with a mixture of DCM (1 mL) and PE (10 mL) to give compound 1B (380 mg, yield: 65.91%) as a light pink solid. ¹H NMR (400 MHz, DMSO-d₆) δ 8.67-8.32 (m, 1H), 7.42-7.25 (m, 6H), 7.23-7.09 (m, 4H), 5.78-5.71 (m, 1H), 4.64-4.40 (m, 1H), 4.14-4.07 (m, 0.7H), 3.79-3.75 (m, 0.4H), 2.88-2.76 (m, 1H), 2.65-2.57 (m, 1H).

To a mixture of compound 1B (100 mg, 272.3 umol) in DCM (15 mL) and DMSO (1 mL) was added DMP (808.5 mg, 1.91 mmol) in one portion under N₂, and then the mixture was stirred at 25° C. for 1 hour. The mixture was quenched with sat. NaHCO₃ (15 mL) and sat. Na₂S₂O₃ (15 mL). The mixture was stirred for 0.5 hour, diluted with dichloromethane (50 mL). The organic layer was washed with water (20 mL×2), dried over Na₂SO₄, filtered and concentrated under reduced pressure to give a white solid, which was purified by triturating with 2-isopropoxypropane (5 mL) to afford compound 1 (60 mg, yield: 60.33%) as a white solid. ¹H NMR (400 MHz, DMSO-d₆) δ 9.20 (d, J=7.6 Hz, 1H), 8.17 (s, 1H), 7.89 (s, 1H), 7.47-7.39 (m, 3H), 7.33-7.27 (m, 4H), 7.25-7.19 (m, 1H), 5.58-5.50 (m, 1H), 3.24-3.17 (m, 1H), 2.84-2.74 (m, 1H). MS (ESI) m/z (M+1)⁺364.9.

### Example 2

### (S)-2,6-dichloro-n-(4-(cyclopropylamino)-3,4-dioxo-1-phenylbutan-2-yl)benzamide (2)

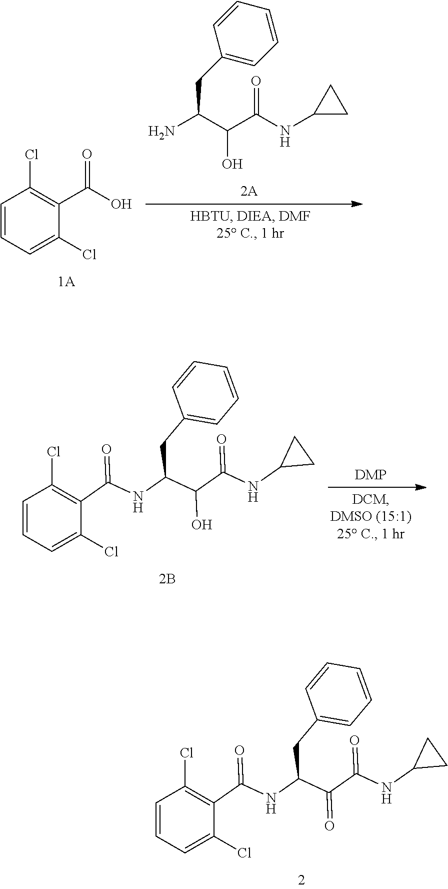 [SMILES: O=C(O)c1c(Cl)cccc1Cl ; N[C@@H](Cc1ccccc1)C(O)C(=O)NC1CC1 ; O=C(N[C@@H](Cc1ccccc1)C(O)C(=O)NC1CC1)c1c(Cl)cccc1Cl ; O=C(NC1CC1)C(=O)[C@H](Cc1ccccc1)NC(=O)c1c(Cl)cccc1Cl]

Compound 2 was prepared following the procedure of Example 1 using the corresponding intermediate 2A and 2,6-dichlorobenzoic acid. ¹H NMR (400 MHz, DMSO-d₆) δ 9.23 (d, J=7.6 Hz, 1H), 8.89 (d, J=5.2 Hz, 1H), 7.45-7.36 (m, 3H), 7.31-7.25 (m, 4H), 7.22-7.18 (m, 1H), 5.53-5.42 (m, 1H), 3.22-3.15 (m, 1H), 2.81-2.74 (m, 2H), 0.69-0.58 (m, 4H). MS (ESI) m/z (M+1)⁺ 405.1.

### Example 3

### (S)—N-(4-amino-3,4-dioxo-1-phenylbutan-2-yl)-2,4,6-trifluorobenzamide (3)

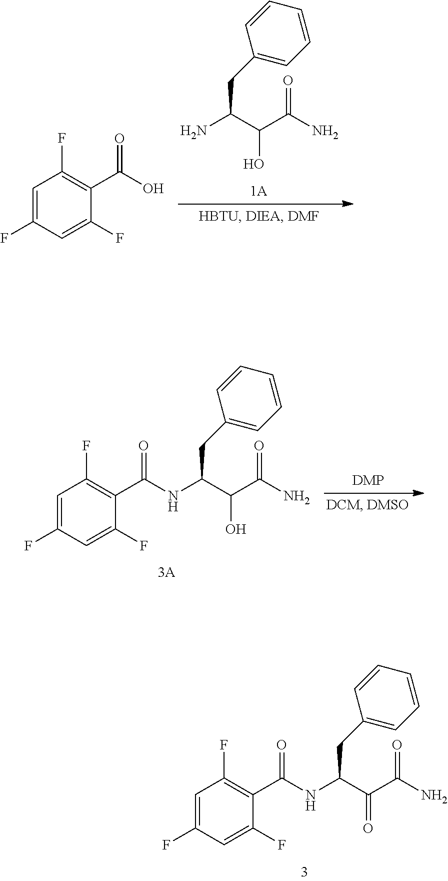 [SMILES: O=C(O)c1c(F)cc(F)cc1F ; NC(=O)C(O)[C@@H](N)Cc1ccccc1 ; NC(=O)C(O)[C@H](Cc1ccccc1)NC(=O)c1c(F)cc(F)cc1F ; NC(=O)C(=O)[C@H](Cc1ccccc1)NC(=O)c1c(F)cc(F)cc1F]

Compound 3 was prepared following the procedure of Example 1 using the corresponding intermediate 1A and 2,4,6-trifluorobenzoic acid. ¹H NMR (400 MHz, DMSO-d₆) δ 9.19 (d, J=7.5 Hz, 1H), 8.14 (s, 1H), 7.86 (s, 1H), 7.30-7.19 (m, 7H), 5.41-5.34 (m, 1H), 3.17 (dd, J=3.4, 14.0 Hz, 1H), 2.75 (dd, J=10.0, 14.0 Hz, 1H). MS (ESI) m/z (M+H)⁺ 351.1.

### Example 4

### (S)—N-(4-amino-3,4-dioxo-1-phenylbutan-2-yl)-4-fluorobenzamide (4)

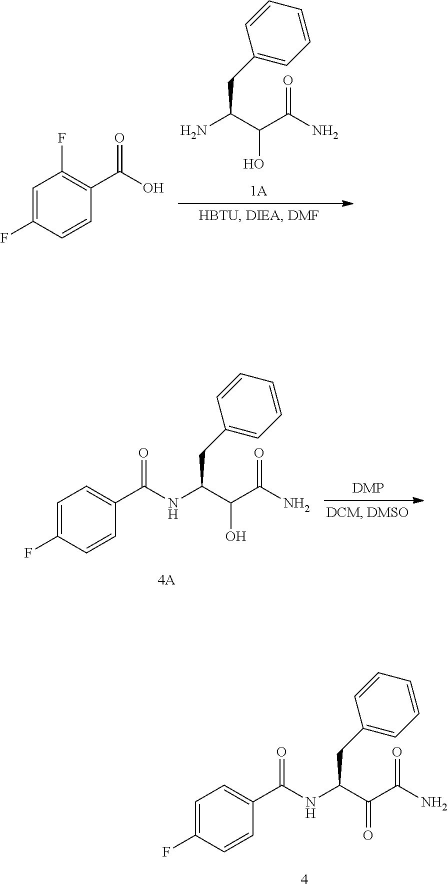 [SMILES: O=C(O)c1ccc(F)cc1F ; NC(=O)C(O)[C@@H](N)Cc1ccccc1 ; NC(=O)C(O)[C@H](Cc1ccccc1)NC(=O)c1ccc(F)cc1 ; NC(=O)C(=O)[C@H](Cc1ccccc1)NC(=O)c1ccc(F)cc1]

Compound 4 was prepared following the procedure of Example 1 using the corresponding intermediate 1A and 4-fluorobenzoic acid. ¹H NMR (400 MHz, DMSO-d₆) δ 8.89 (br d, J=7.0 Hz, 1H), 8.09 (br s, 1H), 7.90-7.78 (m, 3H), 7.35-7.18 (m, 7H), 5.35 (br s, 1H), 3.21 (br d, J=11.5 Hz, 1H), 2.96-2.85 (m, 1H). MS (ESI) m/z (M+H)⁺ 315.1.

### Example 5

### N-(4-amino-3,4-dioxo-1-phenylbutan-2-yl)-9H-xanthene-9-carboxamide (5)

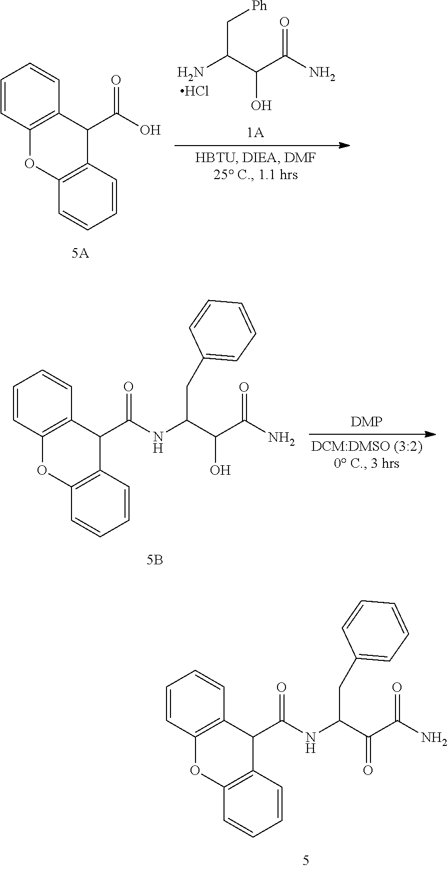 [SMILES: O=C(O)C1c2ccccc2Oc2ccccc21 ; Cl ; NC(=O)C(O)C(N)Cc1ccccc1 ; NC(=O)C(O)C(Cc1ccccc1)NC(=O)C1c2ccccc2Oc2ccccc21 ; NC(=O)C(=O)C(Cc1ccccc1)NC(=O)C1c2ccccc2Oc2ccccc21]

A mixture of compound 5A (250 mg, 1.11 mmol) and compound 1A (305.9 mg, 1.33 mmol, HCl) in DMF (3 mL) was added HBTU (502.9 mg, 1.33 mmol) for 0.1 h, then was added DIEA (571.3 mg, 4.42 mmol), the mixture was stirred at 25° C. for 1 hour under N₂ atmosphere. The residue was purified by preparatory-HPLC (basic condition) to afford compound 5B (210 mg) as a white solid. ¹H NMR (400 MHz, DMSO-d₆) δ 8.56-8.26 (m, 1H), 7.64-7.55 (m, 1H), 7.41-7.26 (m, 9H), 7.07 (br d, J=9.8 Hz, 2H), 6.89-6.75 (m, 1H), 6.69-6.39 (m, 1H), 6.12-5.90 (m, 1H), 5.05-4.91 (m, 1H) 4.35-4.18 (m, 1H), 3.95-3.82 (m, 1H), 2.92 (m, 1H), 2.78-2.63 (m, 2H). MS (ESI) m/z (M+H)⁺ 403.2.

A mixture of compound 5B (110 mg, 273.33 umol) in DMSO (4 mL) and DCM (6 mL) was degassed and purged with N₂ for 3 times, and then was added DMP (347.8 mg, 819.99 umol) at 0° C., the mixture was stirred at 0° C. for 3 hours under N₂ atmosphere. The mixture was quenched with sat.NaHCO₃ (80 mL) and sat. Na₂S₂O₃ (80 mL). The mixture was stirred for 0.5 hour. The organic layer was washed with sat. NaHCO₃ (100 mL×2), water (100 mL×2) and brine (100 mL). The combined organic layers were dried over Na₂SO₄, filtered and filtrate was concentrated under reduced pressure to give a residue. The residue was purified by re-crystallization from 2-isopropoxypropane (10 mL). Compound 5 (80 mg, 185.58 umol) was obtained as a white solid. ¹H NMR (400 MHz, DMSO-d₆) δ 8.98-8.91 (m, 1H), 8.11 (s, 1H), 7.84 (s, 1H), 7.33-7.27 (m, 2H), 7.27-7.17 (m, 5H), 7.27-7.17 (m, 1H), 7.09-7.02 (m, 3H), 6.95-6.90 (m, 1H), 6.86-6.82 (m, 1H), 5.20-5.13 (m, 1H), 5.00 (s, 1H), 3.24-3.17 (m, 1H), 2.82-2.74 (m, 1H). MS (ESI) m/z (M+H)⁺ 401.0.

### Example 6

### N-(4-amino-3,4-dioxo-1-phenylbutan-2-yl)-10H-phenoxazine-10-carboxamide (6)

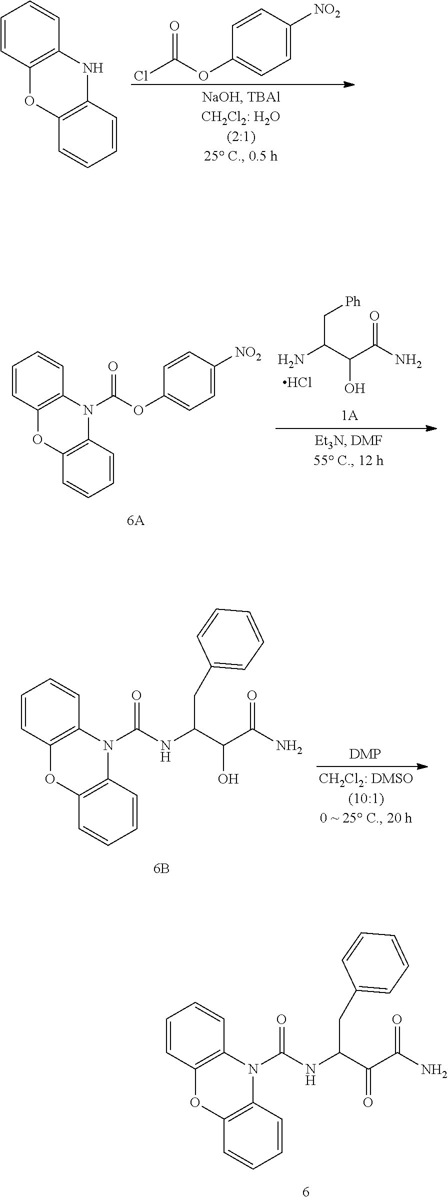

A mixture of 10H-phenoxazine (1 g, 5.46 mmol) in DCM (8 mL) and H₂O (4 mL) was added NaOH (327.5 mg, 8.19 mmol) and TBAI (403.2 mg, 1.09 mmol), and then 4-nitrophenyl carbonochloridate (1.32 g, 6.55 mmol) was added in the mixture was stirred at 25° C. for 0.5 hour. H₂O (50 mL) was added in the mixture, then extracted with CH₂Cl₂ (30 mL×3), the combined organic layers were dried over anhydrous Na₂SO₄, filtered and concentrated under reduced pressure to obtained the crude. The residue was purified by column chromatography (SiO₂, Petroleum ether/Ethyl acetate=I/O to 1:1) to afford compound 6A (380 mg, yield: 19.98%) as yellow solid. ¹H NMR (400 MHz, DMSO-d₆) δ 8.42-8.28 (m, 2H), 8.12 (d, J=9.2 Hz, 2H), 7.81-7.63 (m, 3H), 7.33-7.20 (m, 3H), 6.94 (d, J=9.2 Hz, 2H).

To a solution of compound 6A (380 mg, 1.09 mmol) in DMF (5 mL) was added Et₃N (331.2 mg, 3.27 mmol), then compound 1A (302 mg, 1.31 mmol, HCl) was added and the mixture was stirred at 55° C. for 12 h. It was purified by pre-HPLC (basic condition) to afford compound 6B (50 mg, yield: 11.28%) as gray solid. ¹H NMR (400 MHz, CDCl₃) δ 7.37-7.27 (m, 4H), 7.21 (d, J=7.2 Hz, 1H), 7.16-7.09 (m, 2H), 7.07-7.02 (m, 2H), 7.00-6.84 (m, 5H), 6.28-5.93 (m, 1H), 5.72-5.39 (m, 2H), 4.35-4.14 (m, 2H), 3.46-3.16 (m, 1H), 3.11-2.99 (m, 1H). MS (ESI) m/z (M+H)⁺ 404.1.

A mixture of compound 6B (50 mg, 123.9 umol) in DCM (10 mL) and DMSO (1 mL) was added DMP (368 mg, 867.6 umol) in one portion at 0° C. under N₂, and then the mixture was stirred at 25° C. for 20 hours under N₂ atmosphere. The mixture was quenched with sat. NaHCO₃ (15 mL) and sat. Na₂S₂O₃ (15 mL), and stirred for 20 min, then diluted with dichloromethane (100 mL). The mixture was stirred for 20 min and washed with water (20 mL×2). The combined organic layers were dried over Na₂SO₄ and concentrated under reduced pressure to give the crude product, which was purified by triturated with a mixture of DCM (1 mL) and PE (10 mL) to afford compound 6 (12.3 mg, yield: 24.19%) as yellow solid. ¹H NMR (400 MHz, CDCl₃) δ 7.30-7.24 (m, 5H), 7.15-6.93 (m, 8H), 6.71 (br s, 1H), 5.74 (d, J=6.0 Hz, 1H), 5.47-5.38 (m, 2H), 3.39-3.29 (m, 1H), 3.00-2.94 (m, 1H). MS (ESI) m/z (M+H)⁺ 366.1.

### Example 7

### N-(4-amino-3,4-dioxo-1-phenylbutan-2-yl)dibenzo[b,e][1,4]Dioxine-1-carboxamide (7)

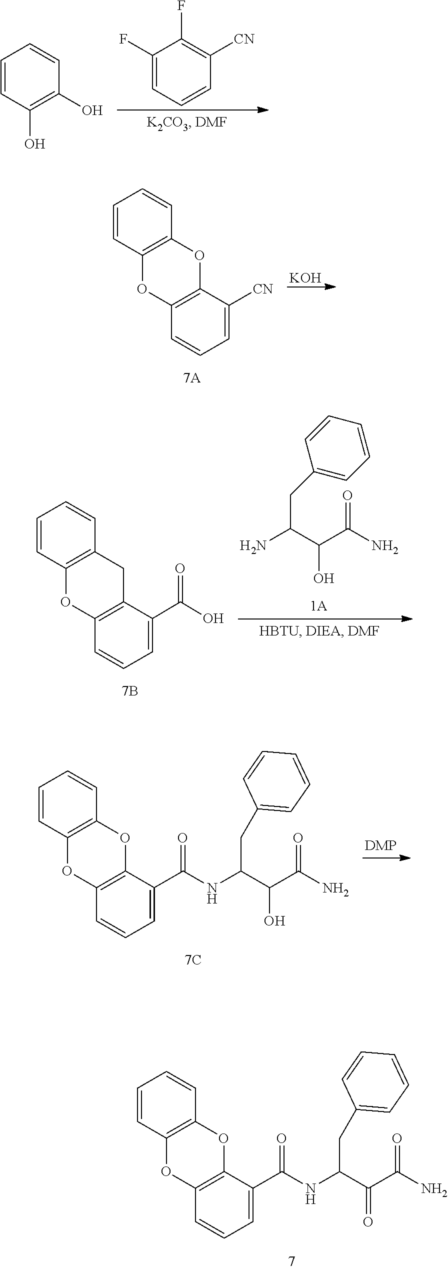

To a mixture of pyrocatechol (100 mg, 908 umol) and 2,3-difluorobenzonitrile (126 mg, 908 umol) in DMF (2.7 mL) and toluene (900 uL) was added K₂CO₃ (377 mg, 2.7 mmol) in one portion under N₂. The mixture was stirred at 130° C. for 12 hours under N₂. The reaction mixture was concentrated to remove toluene. The residue was poured into water (20 mL) and stirred for 10 min. The suspension was filtered and the filtrate cake was washed with H₂O (3 mL) to give compound 7A (140 mg. yield: 73.7%) as a yellow solid. The product was used into the next step without further purification. ¹H NMR (400 MHz, CDCl₃) δ 7.17 (dd, J=1.4, 7.8 Hz, 1H), 7.05 (dd, J=1.5, 8.2 Hz, 1H), 7.01-6.93 (m, 4H), 6.90-6.85 (m, 1H).

To a mixture of compound 7A (140 mg, 669 umol) in ethanediol (3 mL) and H₂O (1 mL) was added KOH (188 mg, 3.4 mmol). The mixture was stirred at 130° C. for 12 hours. Water (20 mL) was added. The mixture was adjusted to pH ˜5 with aqueous HCl (1M). The suspension was filtered and the filtrate cake was washed with H₂O (3 mL) to give compound 7B (130 mg, yield: 85.1%) as a white solid. The product was used into the next step without further purification. ¹H NMR (400 MHz, DMSO-d₆) δ 13.12 (br s, 1H), 7.33 (d, J=7.7 Hz, 1H), 7.13 (d, J=7.1 Hz, 1H), 7.05-6.92 (m, 5H).

To a mixture of compound 7B (120 mg, 526 umol), compound 1A (133 mg, 578 umol) and HBTU (239 mg, 631 umol) in DMF (3 mL) was added DIPEA (272 mg, 2.10 mmol), the mixture was stirred at 15° C. for 0.5 hr. The solid was filtered and washed with methanol (5 mL×3) to give compound 7C (130 mg, yield: 61.1%) as white solid. ¹H NMR (400 MHz, DMSO-d₆) δ 8.15 (br dd, J=8.9, 17.7 Hz, 1H), 7.45-7.34 (m, 2H), 7.34-7.24 (m, 4H), 7.23-7.09 (m, 3H), 7.08-6.95 (m, 5H), 6.17-5.85 (m, 1H), 4.67-4.53 (m, 1H), 4.13-3.90 (m, 1H), 3.00-2.74 (m, 2H).

A mixture of compound 7C (60 mg, 148 umol) and DMP (252 mg, 593 umol) in DCM (15 mL), DMSO (2 mL) was stirred at 15° C. for 1 hr. The mixture was diluted DCM (20 mL), quenched with sat. NaHCO₃ (20 mL), sat. Na₂S₂O₃ (20 mL) and stirred for 20 min, the mixture was extracted with DCM (20 mL×4), the combined organic phase was washed with water (20 mL), brine (20 mL), dried over Na₂SO₄, filtered and concentrated. The residue was stirred in isopropyl ether (10 mL) for 20 min, the solid was filtered and dried to give compound 7 (35.3 mg, yield: 59.1%) as white solid. ¹H NMR (400 MHz, DMSO-d₆) δ 8.61 (br d, J=7.3 Hz, 1H), 8.17 (br s, 1H), 7.91 (br s, 1H), 7.37-7.21 (m, 5H), 7.16-7.07 (m, 2H), 7.07-6.97 (m, 4H), 6.78-6.72 (m, 1H), 5.52-5.43 (m, 1H), 3.26 (br dd, J=4.1, 14.0 Hz, 1H), 3.00 (br dd, J=9.2, 14.0 Hz, 1H). MS (ESI) m/z (M+H)⁺ 403.1.

### Example 8

### N-(4-amino-3,4-dioxo-1-phenylbutan-2-yl)-9H-carbazole-9-carboxamide (8)

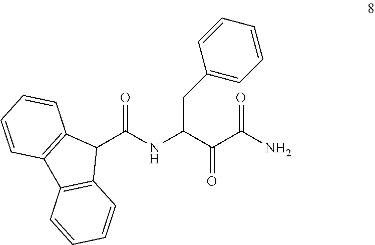 [SMILES: NC(=O)C(=O)C(Cc1ccccc1)NC(=O)C1c2ccccc2-c2ccccc21]

Compound 8 was prepared following the procedure of Example 6 using the intermediate 1A and 9H-carbazole. ¹H NMR (400 MHz, CDCl₃) δ 8.83 (d, J=7.6 Hz, 1H), 8.29 (s, 1H), 8.16 (d, J=7.6 Hz, 1H), 7.99 (s, 1H), 7.61 (d, J=8.4 Hz, 1H), 7.48-7.26 (m, 10H), 5.57-5.44 (m, 1H), 3.39 (s, 1H), 3.01-2.83 (m, 1H). MS (ESI) m/z (M+1)⁺ 386.1.

### Example 9

### N-(4-amino-3,4-dioxo-1-phenylbutan-2-yl)dibenzo[b,d]furan-4-carboxamide (9)

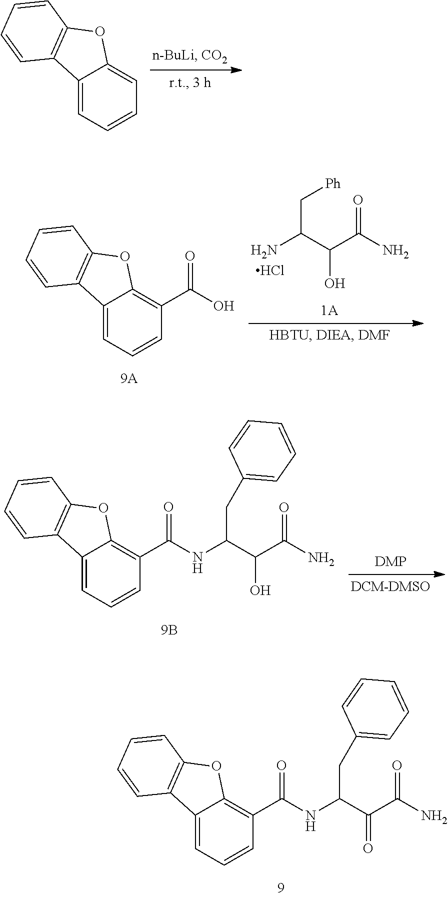 [SMILES: c1ccc2c(c1)oc1ccccc12 ; O=C(O)c1cccc2c1oc1ccccc12 ; Cl ; NC(=O)C(O)C(N)Cc1ccccc1 ; NC(=O)C(O)C(Cc1ccccc1)NC(=O)c1cccc2c1oc1ccccc12 ; NC(=O)C(=O)C(Cc1ccccc1)NC(=O)c1cccc2c1oc1ccccc12]

Dibenzo[b,d]furan (5.00 g, 29.73 mmol) was dissolved in THF (25 ml) and cooled to −78° C. with stirring, t-BuLi (12.0 ml, 62.50 mmol of a 2.50M solution in hexanes) was added dropwise with stirring to give an orange-yellow precipitate. After complete addition the mixture was allowed to warm to room temperature and stirred for 3 h. The orange-brown solution was then cooled to −78° C. and poured onto excess CO₂ (s) covered with anhydrous MTBE. The resulting white precipitate was allowed to stand at room temperature for 1 h. The product was extracted into 2M NaOH and the resulting aqueous phase re-acidified with concentrated HCl before extracting into ethyl acetate. This organic phase was then dried over sodium sulfate, filtered and the solvent evaporated under reduced pressure to give the compound 9A (1.30 g, 20.61% yield) as white solid. ¹H NMR (400 MHz, DMSO-d₆) δ 13.32 (br s, 1H), 8.42 (d, J=7.2 Hz, 1H), 8.22 (d, J=7.6 Hz, 1H), 8.04 (d, J=7.2 Hz, 1H), 7.81 (d, J=8.4 Hz, 1H), 7.60-7.54 (m, 1H), 7.53-7.49 (m, 1H), 7.47-7.44 (m, 1H). MS(ESI) m/z (M+1)⁺ 213.0.

To a mixture of compound 9A (200 mg, 942.51 umol) and compound 1A (261 mg, 1.13 mmol, HCl) in DMF (4 mL) was added HBTU (536 mg, 1.41 mmol) in one portion at 25° C. under N₂. The mixture was stirred at 25° C. for 0.1 hour, and then DIEA (365 mg, 2.83 mmol, 494 uL) was added. The resultant mixture was stirred at 25° C. for 3 hrs. The mixture was purified by preparatory-HPLC (basic condition) to afford compound 9B (160 mg, 43.39% yield) as white solid. ¹H NMR (400 MHz, DMSO-d₆) δ 8.32-8.27 (m, 1H), 8.22-8.18 (m, 1H), 8.15-7.89 (m, 1H), 7.91-7.79 (m, 2H), 7.61-7.57 (m, 1H), 7.50-7.43 (m, 4H), 7.35-7.18 (m, 6H), 6.26-5.97 (m, 1H), 4.68-4.57 (m, 1H), 4.18-4.16 (m, 1H), 3.93-3.92 (m, 1H). MS(ESI) m/z (M+1)⁺ 389.1.

To a solution of compound 9B (150 mg, 386.18 umol) in DMSO (4 mL) and CH₂Cl₂ (4 mL) was added DMP (491 mg, 1.16 mmol) under N₂ atmosphere, the mixture was stirred at 0° C. for 1.5 hours. The mixture was quenched with sat. NaHCO₃ (20 mL) and sat. Na₂S₂O₃ (20 mL). The mixture was stirred for 0.5 hour, diluted with dichloromethane (100 mL). The organic layer was washed with NaHCO₃ (30 mL×3), water (20 mL×3) and brine (30 mL×3), dried over Na₂SO₄, filtered and the filtrate was concentrated under reduced pressure to give the residue. The product was purified by triturated in isopropyl ether (12 mL) to afford compound 9 (30 mg, 20.10% yield) as white solid. ¹H NMR (400 MHz, CDCl₃) δ 8.19-8.17 (m, 2H), 8.08 (d, J=6.8 Hz, 1H), 7.97 (d, J=7.6 Hz, 1H), 7.52 (s, 2H), 7.47-7.40 (m, 2H), 7.34 (s, 5H), 6.85 (s, 1H), 5.83 (s, 1H), 5.61 (s, 1H), 3.54-3.52 (m, 1H), 3.20-3.40 (m, 1H). MS (ESI) m/z (M+1)⁺387.0.

### Example 10

### N-(4-amino-3,4-dioxo-1-phenylbutan-2-yl)-9H-fluorene-9-carboxamide (10)

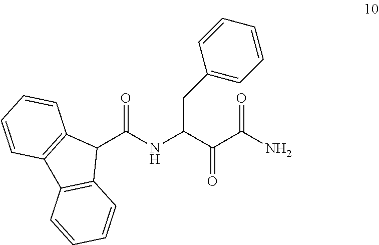 [SMILES: NC(=O)C(=O)C(Cc1ccccc1)NC(=O)C1c2ccccc2-c2ccccc21]

Compound 10 was prepared following the procedure of Example 6 using the intermediate 1A and 9H-fluorene-9-carboxylic acid. ¹H NMR (400 MHz, CDCl₃) δ 7.81-7.76 (m, 2H), 7.61-7.50 (m, 2H), 7.48-7.41 (m, 2H), 7.37-7.30 (m, 2H), 7.18-7.04 (m, 3H), 6.72-6.60 (m, 3H), 5.72 (br s, 1H), 5.46-5.29 (m, 2H), 4.76 (s, 1H), 3.24-3.14 (m, 1H), 2.99-2.90 (m, 1H). MS (ESI) m/z (M+H)⁺ 385.1.

### Example 11

### N-(4-amino-3,4-dioxo-1-phenylbutan-2-yl)-9-methyl-9H-carbazole-4-carboxamide (11)

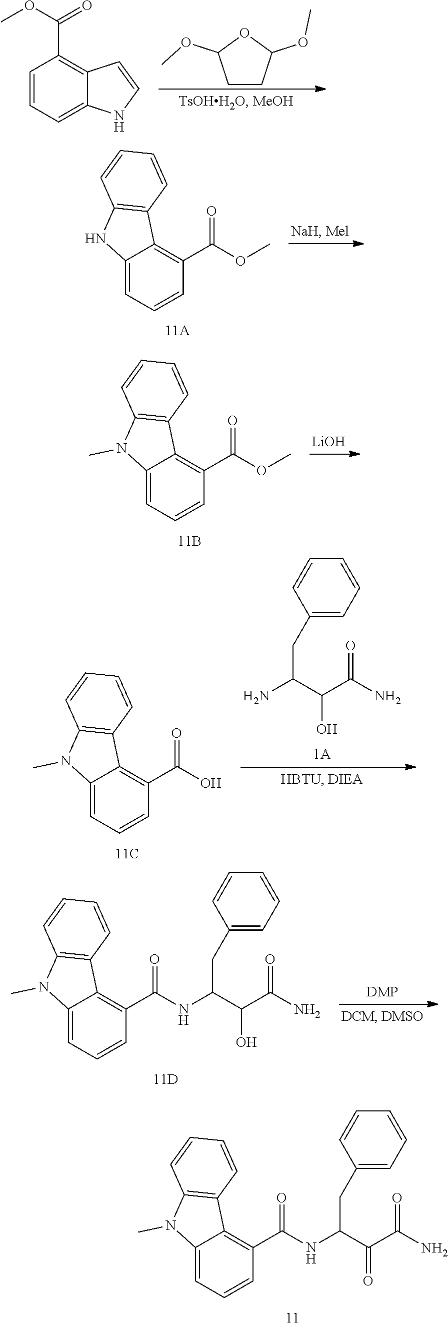

A mixture of methyl 1H-indole-4-carboxylate (2 g, 11.4 mmol) and 2,5-dimethoxytetrahydrofuran (1.96 g, 14.9 mmol) in MeOH (50 mL) was added TsOH.H₂O (1.09 g, 5.71 mmol). The reaction mixture was stirred at 65° C. for 16 hrs. The reaction mixtures were concentrated. The crude product was purified by silica gel column chromatography (petroleum ether:ethyl acetate=20:1˜5:1) to give compound 11A (220 mg, yield: 4.28%) as yellow solid. ¹H NMR (400 MHz, CDCl₃) δ 8.78 (d, J=8.2 Hz, 1H), 8.19 (br s, 1H), 7.80 (d, J=7.6 Hz, 1H), 7.55 (d, J=8.1 Hz, 1H), 7.42-7.36 (m, 2H), 7.23-7.15 (m, 2H), 4.00 (s, 3H).

A solution of compound 11A (200 mg, 888 umol) in DMF (2 mL) was added NaH. (53.3 mg, 1.33 mmol, 60%) at 0° C. The reaction mixture was stirred at 0° C. for 0.5 hr. Then MeI (252 mg, L₇₈ mmol) was added to the reaction mixture. The reaction mixture was allowed to warm to 15° C. with stirring for 16 hr. Saturated. NH₄Cl (10 mL) was added to the reaction mixture. The product was extracted with EtOAc (10 mL×2). The combined organic layer was concentrated and purified by preparatory-TLC (PE:EA=5:1, R_{f}=0.6) to give compound 11B (150 mg, yield: 70.6%) as yellow solid. ¹H NMR (400 MHz, CDCl₃) δ 8.90 (d, J=8.2 Hz, 1H), 7.89 (dd, J=0.9, 7.5 Hz, 1H), 7.64 (d, J=7.5 Hz, 1H), 7.58-7.51 (m, 2H), 7.46 (d, J=8.3 Hz, 1H), 7.33-7.29 (m, 1H), 4.10 (s, 3H), 3.92 (s, 3H).

A solution of compound 11B (150 mg. 627 umol) in MeOH (5 mL) and H₂O (1.00 mL) was added NaOH (50.2 mg, 1.25 mmol). The reaction mixture was stirred at 50° C. for 16 hrs. 1M HCl was added drop-wise until pH ˜6. The solvent was evaporated to give crude compound 11C (140 mg, crude) as white solid. The crude product was used in the next step without purification.

A mixture of compound 11C (140 mg, 622 umol) and intermediate 1A (1.43 mg, 622 umol, HCl salt) in DMF (2 mL) was added HBTU (354 rig, 932 umol) and DIEA (241 mg, 1.86 mmol). The reaction mixture was stirred at 15° C. for 16 hrs. The reaction mixture was filtered. The crude product was purified by prep-HPLC (FA) to give compound 11D (160 mg, yield: 64.1%) as white solid. ¹H NMR (400 MHz, DMSO-d₆) δ 8.27 (d, J=9.0 Hz, 1H), 7.97-7.86 (m, 1H), 7.67 (dd, J=5.3, 7.8 Hz, 1H), 7.55 (d, J=8.2 Hz, 1H), 7.51-7.14 (m, 10H), 7.05 (q, J=7.7 Hz, 1H), 7.09-7.00 (m, 1H), 5.91-5.77 (m, 1H), 4.83-4.67 (m, 1H), 4.22-3.99 (m, 1H), 3.88 (d, J=2.4 Hz, 3H), 3.07-2.77 (m, 2H).

A solution of compound 11D (140 mg, 349 umol) in DCM (20 mL) was added DMP (592 mg, 1.39 mmol). Then the reaction mixture was stirred at 15° C. for 16 hrs. The mixture was diluted with DCM (20 mL), quenched by addition sat. NaHCO₃ (30 mL) and sat. Na₂S₂O₃ (30 mL) at 15° C., and then the mixture was stirred until the solution was clear, and extracted with DCM (30 mL×2). The combined organic layers were washed with H₂O (20 mL) and brine (20 mL), dried over Na₂SO₄, filtered and concentrated under reduced pressure to give a residue. The residue was purified by trituration in isopropyl ether solvent (10 mL). The mixture was filtered and dried to give compound 11 (84.2 mg, yield: 60.5%) as white solid. ¹H NMR (400 MHz, CDCl₃) δ 8.26 (d, J=7.9 Hz, 1H), 7.46-7.30 (m, 4H), 7.21-7.07 (m, 7H), 6.74 (br s, 1H), 6.54 (br d, J=7.0 Hz, 1H), 5.80 (dt, J=5.2, 7.2 Hz, 1H), 5.46 (br s, 1H), 3.79 (s, 3H), 3.51 (dd, J=5.1, 14.2 Hz, 1H), 3.23 (dd, J=7.6, 14.2 Hz, 1H). MS (ESI) m/z (M+H)⁺ 400.1.

### Example 12

### N-(4-amino-3,4-dioxo-1-phenylbutan-2-yl)-9-methyl-9H-carbazole-1-carboxamide (12)

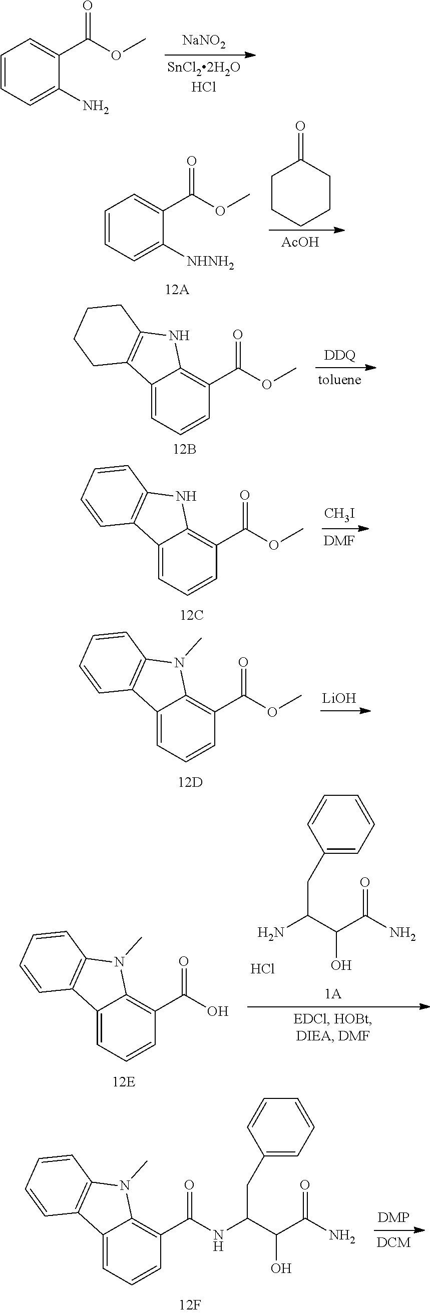

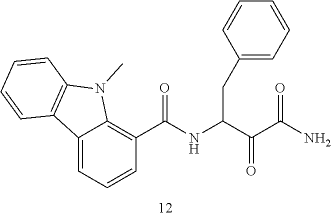 [SMILES: Cn1c2ccccc2c2cccc(C(=O)NC(Cc3ccccc3)C(=O)C(N)=O)c21]

To a solution of methyl 2-aminobenzoate (10 g, 66.15 mmol) in HCl (100 mL) at 0° C. was added a solution of NaNO₂ (4.66 g, 67.48 mmol) in H₂O (100 mL) dropwise. The mixture was stirred at 0° C. for 0.5 h. Then a solution of SnCl₂.2H₂O (29.85 g, 132.31 mmol) in HCl (50 mL) was added. The mixture was stirred at 25° C. for 2 h. The solid was filtered, washed with H₂O (200 mL), collected and dried in vacuo to afford compound 12A (7.8 g, yield: 56.56%) as white solid.

A solution of compound 12A (2 g, 9.87 mmol) in AcOH (20 mL) was heated to 80° C. Then cyclohexanone (970 mg, 9.87 mmol) was added to the solution dropwise. Then the solution was heated to 100° C. and stirred for 2 h. The reaction was cooled to room temperature and H₂O (20 mL) was added. The solid was filtered, collected and dried in vacuo to give compound 12B (1.3 g, yield: 49.69%) as purple solid. MS (ESI) m/z (M+H)⁺ 229.9.

To a solution of compound 12B (1.3 g, 5.67 mmol) in toluene (40 mL) was added DDQ (1.54 g, 6.80 mmol) in one portion. The mixture was stirred at 100° C. for 12 h. The solid was filtered. The filtrate was collected and concentrated. The residue was purified by column (PE:EA=5:1) to give compound 12C (360 mg, yield: 28.19%) as light yellow solid. ¹H NMR (CDCl₃, 400 MHz): δ 9.92 (br. s, 1H), 8.29-8.22 (m, 1H), 8.13-8.05 (m, 2H), 7.59-7.44 (m, 2H), 7.30-7.20 (m, 2H), 4.03 (s, 3H).

To a solution of compound 12C (360 mg, 1.60 mmol) in DMF (5 mL) was added NaH (320 mg, 7.99 mmol, 60% purity) portionwise, followed by addition of CH₃I (0.2 mL, 3.20 mmol). The mixture was stirred at 25° C. for 12 h. The mixture was quenched with 1N HCl until pH ˜4, diluted with H₂O (30 mL), extracted with EtOAc (20 mL×3). The organics were collected, washed with brine (20 mL), dried with Na₂SO₄, filtered and concentrated to give compound 12D (380 mg, crude) as yellow oil, which was used directly for the next step without further purification. MS (ESI) m/z (M+H)⁺ 239.8.

To a solution of compound 12D (380 mg, 1.59 mmol) in THF (3 mL), MeOH (3 mL), and H₂O (3 mL) was added LiOH.H₂O (335 mg, 7.94 mmol). The mixture was stirred at 25° C. for 48 h. The mixture was acidified with 1N HCl to pH ˜4, diluted with H₂O (20 mL), extracted with EtOAc (15 mL×2). The organics were collected, washed with brine (20 mL), dried with Na₂SO₄, filtered and concentrated. The residue was purified by SFC (column: AD (250 mm×30 mm, 5 um); mobile phase: [0.1% NH₃H₂O/EtOH]) (RT: 6.114 min). The pure fraction was collected and concentrated. The residue was dissolved in H₂O (10 mL), acidified with 1N HCl to pH ˜4. The mixture was extracted with EtOAc (15 mL×2). The organics were collected, washed with brine (20 mL), dried with Na₂SO₄, filtered and concentrated to give compound 12E (310 mg, yield: 86.66%) as white solid. ¹H NMR (CDCl₃, 400 MHz): δ 8.36-8.29 (m, 1H), 8.14-8.06 (m, 2H), 7.55-7.45 (m, 2H), 7.34-7.22 (m, 2H), 4.02 (s, 3H).

To a solution of compound 12E (310 mg, 1.38 mmol) and intermediate 1A (477 mg, 2.06 mmol) in DMF (10 mL) was added DIEA (0.6 mL, 3.44 mmol), HOBt (56 mg, 412.89 umol) and EDCI (396 mg, 2.06 mmol). The mixture was stirred at 25° C. for 48 h. The solvent was removed in vacuo. The residue was dissolved in EtOAc (40 mL), washed with 1N HCl (40 mL). The organics were collected, washed with saturated NaHCO₃ (40 mL), brine (40 mL), dried with Na₂SO₄, filtered and concentrated. The residue was purified by prep-HPLC (Neutral) to give compound 12F (320 mg, yield: 57.40%) as white solid. MS (ESI) m/z (M+H)⁺ 401.9.

To a solution of compound 12F (150 mg, 373.64 umol) in DCM (20 mL) and DMSO (3 mL) was added DESS-MARTIN PERIODINANE (476 mg, 1.12 mmol). The mixture was stirred at 25° C. for 2 h. The reaction was diluted with DCM (30 mL), quenched with a solution of 10% aqueous Na₂S₂O₃ and saturated NaHCO₃ (v/v=1/1) (60 mL). The solid was filtered, collected, washed with H₂O (10 mL). The solid was filtered, collected, and dried in vacuo to give compound 12 (28 mg, yield: 18.05%) as white solid. MS (ESI) m/z (M+H)⁺ 400.1. ¹H NMR (DMSO-d₆, 400 MHz): δ 9.16 (d, J=8.0 Hz, 1H), 8.26-8.22 (m, 1H), 8.20 (br. s, 1H), 8.15 (d, J=7.6 Hz, 1H), 7.91 (br. s, 1H), 7.57-7.52 (m, 1H), 7.49-7.43 (m, 1H), 7.38-7.30 (m, 4H), 7.28-7.16 (m, 4H), 5.55-5.48 (m, 1H), 3.49 (s, 3H), 3.30-3.24 (m, 1H), 2.87-2.78 (m, 1H).

### Example 13

### N-(4-amino-3,4-dioxo-1-phenylbutan-2-yl)-2-chloro-1-naphthamide (13)

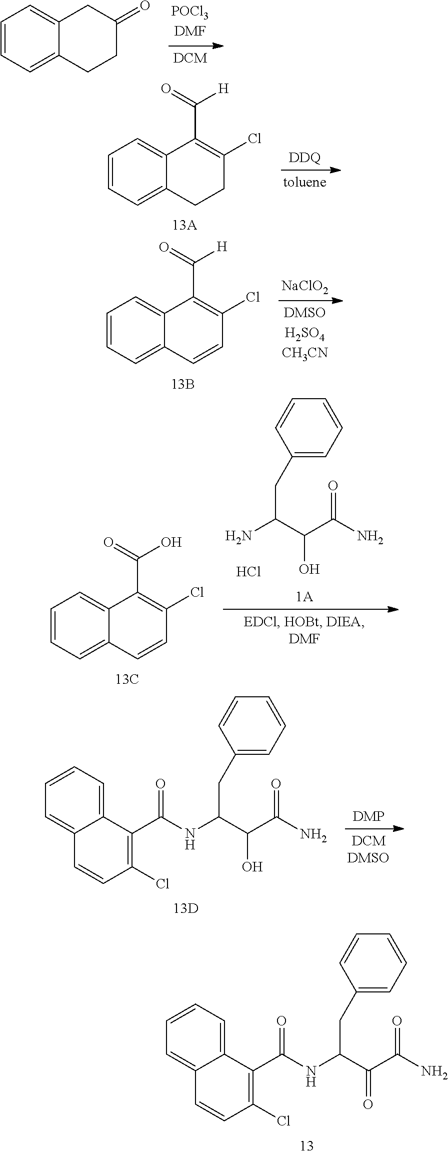

DMF (1.67 g, 22.85 mmol, 1.76 mL) was cooled to 0° C., POCl₃ (2.5 mL, 26.74 mmol) was added dropwise. The mixture was stirred at 0° C. for 0.5 h. Then DCM (10 mL) was added. The mixture was stirred at 15° C. for 2 h. Then a solution of 3,4-dihydronaphthalen-2(1H)-one (1 g, 6.84 mmol) in DCM (5 mL) was added. The mixture was stirred at 15° C. for 12 h. The reaction was diluted with DCM (20 mL), quenched with H₂O (30 mL) dropwise carefully. The organics were collected, washed with saturated NaHCO₃ (30 mL), dried with Na₂SO₄, filtered and concentrated. The residue was purified by column (PE:EA=10:1) to give compound 13A (940 mg, yield: 71.33%) as yellow oil. ¹H NMR (CDCl₃, 400 MHz): δ 10.47 (s, 1H), 8.04-7.99 (m, 1H), 7.25-7.05 (m, 3H), 2.92-2.84 (m, 4H).

The solution of compound 13A (500 mg, 2.60 mmol) and DDQ (590 mg, 2.60 mmol) in toluene (20 mL) was stirred at 90° C. for 12 h. Then additional DDQ (590 mg, 2.60 mmol) was added. The mixture was stirred at 90° C. for 48 h. The solid was filtered. The filtrate was collected and concentrated. The residue was purified by column (PE:EA=10:1) to give compound 13B (380 mg, yield: 57.60%) as white solid. ¹H NMR (CDCl₃, 400 MHz): δ 10.91 (s, 1H), 9.16-9.13 (m, 1H), 8.02-7.55 (m, 1H), 7.67-7.63 (m, 1H), 7.70-7.62 (m, 1H), 7.60-7.55 (m, 1H), 7.55-7.45 (m, 1H).

To a solution of compound 13B (380 mg, 1.99 mmol) and DMSO (0.19 mL, 2.41 mmol) in CH₃CN (10 mL) and H₂O (0.3 mL) at 0° C. was added H₂SO₄ (0.06 mL, 1.10 mmol) dropwise. After addition, a solution of NaClO₂ (270 mg, 2.99 mmol) in H₂O (1.7 mL) was added. The mixture was stirred at 0° C. for 2 h. The mixture was washed with H₂O (10 mL), extracted with EtOAc (15 mL×2). The organics were collected, dried with Na₂SO₄, filtered and concentrated. The crude was purified by SFC (0.1% NH₃H₂O EtOH) (RT: 2.304 min). The main peak was collected and concentrated. The residue was dissolved in H₂O (10 mL), acidified with 1N HCl to pH ˜4, extracted with EtOAc (15 mL×2). The organics were collected, dried with Na₂SO₄, filtered and concentrated to give compound 13C (270 mg, yield: 65.55%) as light yellow solid. ¹H NMR (CDCl₃, 400 MHz): δ 8.05-7.96 (m, 1H), 7.95-7.80 (m, 2H), 7.68-7.45 (m, 3H).

To a solution of compound 13C (260 mg, 1.26 mmol) and intermediate 1A (436 mg, 1.89 mmol) in DMF (10 mL) was added DIEA (0.55 mL, 3.15 mmol), HOBt (52 mg, 377.50 umol) and EDCI (362 mg, 1.89 mmol). The mixture was stirred at 25° C. for 12 h. The solvent was removed in vacuo. The residue was dissolved in EtOAc (30 mL), washed with 1N HCl (30 mL). The organics were collected, washed with saturated NaHCO₃ (30 mL), brine (30 mL), dried with Na₂SO₄, filtered and concentrated. The residue was purified by prep-HPLC to give compound 13D (160 mg, yield: 31.42%) as white solid. MS (ESI) m/z (M+Na)⁺ 404.9.

To a solution of compound 13D (160 mg, 417.93 umol) in DCM (20 mL) and DMSO (3 mL) was added DMP (532 mg, 1.25 mmol). The mixture was stirred at 25° C. for 40 min. The mixture diluted with DCM (20 mL), quenched with a solution of 10% aqueous Na₂S₂O₃ and saturated NaHCO₃ (v/v=1/1) (80 mL). The organics were collected, washed with H₂O (40 mL×5), collected and concentrated. The residue was washed with CH₃CN (8 mL). The solid was filtered, collected and dried in vacuo to give compound 13 (65 mg, yield: 38.84%) as white solid. MS (ESI) m/z (M+H)⁺ 381.1. ¹H NMR (DMSO-d₆, 400 MHz): δ 9.30 (d, J=7.6 Hz, 1H), 8.33 (br. s, 1H), 8.09-7.97 (m, 3H), 7.70-7.30 (m, 9H), 5.77-5.68 (m, 1H), 3.38-3.30 (m, 1H), 2.89-2.77 (m, 1H).

### Example 14

### General Synthesis of Compounds 14-36

### Synthetic Scheme A:

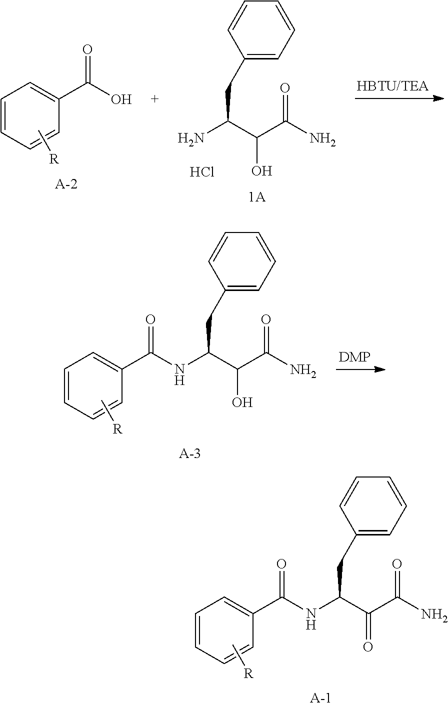

A mixture of acid A-2 (1 equiv.) in DMF was added HBTU (1.5 equiv.) followed by TEA (3 equiv.). The reaction mixture was stirred at 20° C. for 5 mins and intermediate 1A (1 equiv.) was added. The reaction mixture stirred for 3 h, diluted with water, and filtered. Crude product was stirred with EtOAc for 30 min and filtered to afford compound A-3 as off white solid.

To a solution of compound A-2 (1 equiv) in DCM and DMSO was added DMP (2 equiv.). The reaction mixture was stirred at 20° C. for 2 hrs. The reaction mixture was diluted with DCM (10 mL), quenched with sat. NaHCO₃ and 10% aqueous Na₂S₂O₃ at 20° C., stirred for 30 min and extracted with DCM (10 mL×2). The combined organic layers were washed with H₂O (10 mL), brine (10 mL), dried over Na₂SO₄, filtered and concentrated under reduced pressure to afford the crude product. Crude product was purified by flash chromatography using EtOAc/Hexane to afford the desired product A-1.

### Synthetic Scheme B:

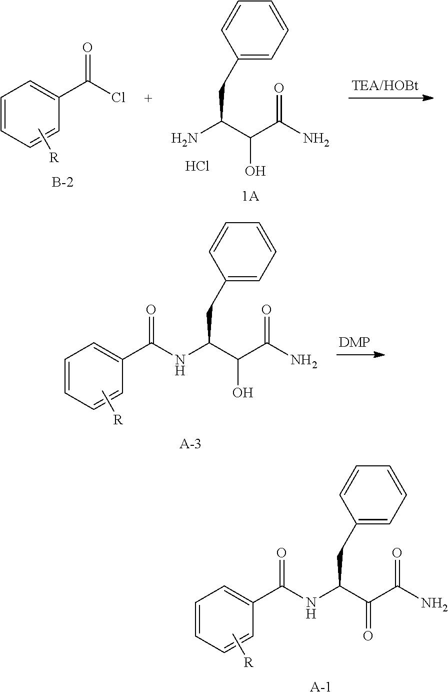

A mixture of acid chloride B-2 (1 equiv.) in DMF was added HOBt (1 equiv.) at 0° C. followed by addition of TEA (3 equiv.). The reaction mixture was stirred at 0° C. for 5 mins and intermediate 1A (1 equiv.) was added. The reaction mixture stirred for 3 h, diluted with water, and filtered. Crude product was stirred with EtOAc for 30 min and filtered to afford compound A-3 as off white solid.

To a solution of compound A-3 (1 equiv) in DCM and DMSO was added DMP (2 equiv.). The reaction mixture was stirred at 20° C. for 2 hrs. The reaction mixture was diluted with DCM (10 mL), quenched with sat. NaHCO₃, and 10% aqueous Na₂S₂O₃ at 20° C., and stirred for 30 min and extracted with DCM (10 mL×2). The combined organic layers were washed with H₂O (10 mL), brine (10 mL), dried over Na₂SO₄, filtered and concentrated under reduced pressure to afford the crude product. Crude product was purified by flash chromatography using EtOAc/Hexane to afford the desired product A-1.

### (S)—N-(4-amino-3,4-dioxo-1-phenylbutan-2-yl)-2-bromo-6-chlorobenzamide (14)

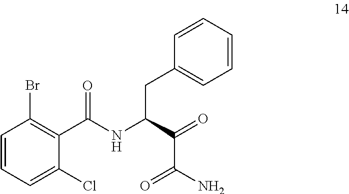 [SMILES: NC(=O)C(=O)[C@H](Cc1ccccc1)NC(=O)c1c(Cl)cccc1Br]

Compound 14: ¹H NMR (400 MHz, DMSO): δ 9.17 (d, 1H), 8.15 (s, 1H), 7.87 (s, 1H), 7.58 (d, 1H), 7.47 (d, 1H), 7.33-7.18 (m, 6H), 5.52 (m, 1H), 3.18 (dd, 1H), 2.79 (dd, 1H) ppm. MS (ESI) m/z (M+H)⁺ 410.9.

### (S)—N-(4-amino-3,4-dioxo-1-phenylbutan-2-yl)-2,6-difluorobenzamide (15)

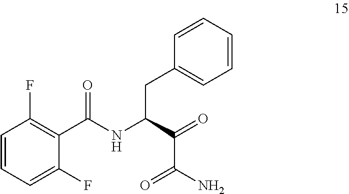 [SMILES: NC(=O)C(=O)[C@H](Cc1ccccc1)NC(=O)c1c(F)cccc1F]

Compound 15: ¹H NMR (400 MHz, DMSO): δ 9.2 (d, 0.6H), 8.25 (d, 0.4H), 8.15 (s, 0.6H), 7.87 (s, 0.6H), 7.55-7.35 (m, 1.4H), 7.3-7.1 (m, 7.4H), 5.41 (m, 0.6H), 4.47 (m, 0.4H), 3.18 (dd, 0.6H), 3.04 (dd, 0.4H), 2.78 (dd, 0.6H), 2.59 (dd, 0.4H), ppm. MS (ESI) m/z (M+H)⁺ 332.3.

### (S)—N-(4-amino-3,4-dioxo-1-phenylbutan-2-yl)-2-fluoro-6-(trifluoromethyl)benzamide (16)

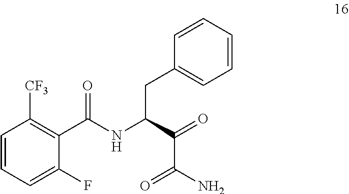 [SMILES: NC(=O)C(=O)[C@H](Cc1ccccc1)NC(=O)c1c(F)cccc1C(F)(F)F]

Compound 16: ¹H NMR (400 MHz, DMSO): δ 9.26 (d, 0.4H), 8.37 (d, 0.6H), 8.16 (s, 0.4H), 7.87 (s, 0.4H), 7.7-7.1 (m, 9.2H), 5.52 (m, 0.4H), 4.55 (m, 0.6H), 3.2-3.05 (m, 1H), 2.78 (dd, 0.4H), 2.89 (dd, 0.6H), ppm. MS (ESI) m/z (M+H)⁺ 383.3.

### (S)—N-(4-amino-3,4-dioxo-1-phenylbutan-2-yl)-5-chloro-2-methoxybenzamide (17)

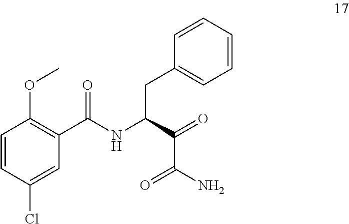 [SMILES: COc1ccc(Cl)cc1C(=O)N[C@@H](Cc1ccccc1)C(=O)C(N)=O]

Compound 17: MS (ESI) m/z (M+H)⁺ 357.

### (S)—N-(4-amino-3,4-dioxo-1-phenylbutan-2-yl)-2,6-dimethoxybenzamide (18)

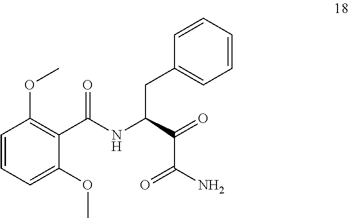 [SMILES: COc1cccc(OC)c1C(=O)N[C@@H](Cc1ccccc1)C(=O)C(N)=O]

Compound 18: ¹H NMR (400 MHz, DMSO): δ 8.46 (d, 0.2H), 8.04 (s, 0.2H), 7.93 (d, 0.8H), 7.79 (s, 0.2H), 7.4-7.1 (m, 7.6H), 6.65-6.58 (m, 2H), 5.34 (m, 0.2H), 4.32 (m, 0.8H), 3.63 (s, 6H), 3.08 (dd, 0.2H), 2.96 (dd, 0.8H), 2.89 (dd, 0.2H), 2.68 (dd, 0.8H), ppm. MS (ESI) m/z (M+H)⁺ 357.

### (S)—N-(4-amino-3,4-dioxo-1-phenylbutan-2-yl)-2-chloro-6-(trifluoromethyl)benzamide (19)

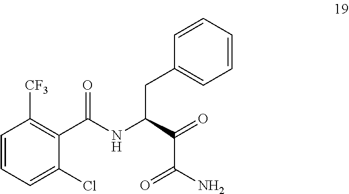 [SMILES: NC(=O)C(=O)[C@H](Cc1ccccc1)NC(=O)c1c(Cl)cccc1C(F)(F)F]

Compound 19: ¹H NMR (400 MHz, DMSO): δ 9.2 (d, 1H), 8.2-7.8 (m, 4H), 7.2-7 (m, 6H), 5.58 (m, 1H), 3.16 (dd, 1H), 2.78 (dd, 1H) ppm. MS (ESI) m/z (M+H)⁺ 399.4.

### (S)—N-(4-amino-3,4-dioxo-1-phenylbutan-2-yl)-2,6-bis(trifluoromethyl)benzamide (20)

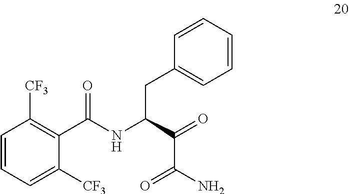 [SMILES: NC(=O)C(=O)[C@H](Cc1ccccc1)NC(=O)c1c(C(F)(F)F)cccc1C(F)(F)F]

Compound 20: ¹H NMR (400 MHz, DMSO): δ 9.25 (d, 1H), 8.15 (s, 1H), 7.87 (s, 1H), 7.78 (d, 1H), 7.71 (d, 1H), 7.6 (t, 1H), 7.3-7.2 (m, 5H), 5.63 (m, 1H), 3.1 (dd, 1H), 2.81 (dd, 1H) ppm. MS (ESI) m/z (M+H)⁺ 433.1.

### (S)—N-(4-amino-3,4-dioxo-1-phenylbutan-2-yl)-3-chloro-[1,1′-biphenyl]-2-carboxamide (21)

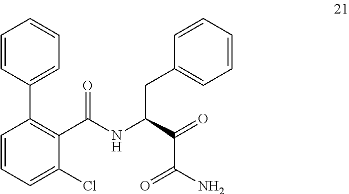 [SMILES: NC(=O)C(=O)[C@H](Cc1ccccc1)NC(=O)c1c(Cl)cccc1-c1ccccc1]

Compound 21: ¹H NMR (400 MHz, DMSO): δ 9.06 (d, 1H), 8.05 (s, 1H), 7.8 (s, 1H), 7.5-7.1 (m, 13H), 5.34 (m, 1H), 2.98 (dd, 1H), 2.65 (dd, 1H) ppm. MS (ESI) m/z (M+H)⁺ 406.9.

### (S)—N-(4-amino-3,4-dioxo-1-phenylbutan-2-yl)-2,5-dichlorobenzamide (22)

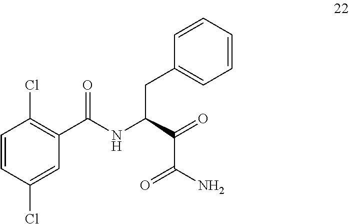 [SMILES: NC(=O)C(=O)[C@H](Cc1ccccc1)NC(=O)c1cc(Cl)ccc1Cl]

Compound 22: ¹H NMR (400 MHz, DMSO): δ 8.99 (d, 1H), 8.08 (s, 1H), 7.82 (s, 1H), 7.45 (m, 2H), 7.3-7.1 (m, 6H), 5.28 (m, 1H), 3.16 (dd, 1H), 2.75 (dd, 1H) ppm. MS (ESI) m/z (M+H)⁺ 364.9.

### (S)—N-(4-amino-3,4-dioxo-1-phenylbutan-2-yl)-[1,1′-biphenyl]-4-carboxamide (23)

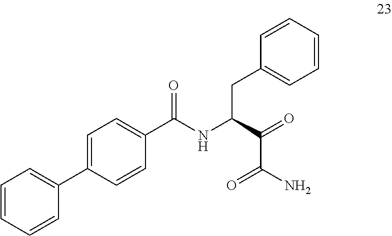 [SMILES: NC(=O)C(=O)[C@H](Cc1ccccc1)NC(=O)c1ccc(-c2ccccc2)cc1]

Compound 23: ¹H NMR (400 MHz, DMSO-d₆): δ 7.6-8.1 (m, 7H), 7-7.6 (m, 8H), 5.3 (m, 1H), 3.3 (d, 2H), 3.0 (m, 1H) ppm. MS (ESI) m/z (M+H)⁺ 373.

### N-(4-amino-3,4-dioxo-1-phenylbutan-2-yl)benzo[d][1,3]dioxole-5-carboxamide (24)

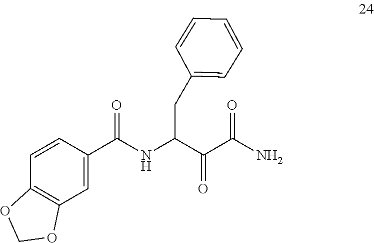 [SMILES: NC(=O)C(=O)C(Cc1ccccc1)NC(=O)c1ccc2c(c1)OCO2]

Compound 24: ¹H NMR (400 MHz, DMSO-d₆): δ 7.05-7.35 (m, 7H), 6.75-6.85 (m, 1H), 6.0 (m, 1H), 3.3 (d, 2H), 2.95-3.0 (m, 1H) ppm. MS (ESI) m/z (M+H)⁺ 341.

### N-(4-amino-3,4-dioxo-1-phenylbutan-2-yl)-3-fluorobenzamide (25)

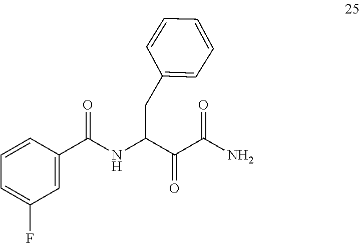 [SMILES: NC(=O)C(=O)C(Cc1ccccc1)NC(=O)c1cccc(F)c1]

Compound 25: ¹H NMR (400 MHz, DMSO): δ 8.9 (d, 1H), 8.05 (s, 1H), 7.78 (s, 1H), 7.58 (d, 1H), 7.51 (d, 1H), 7.46 (d, 1H), 7.33 (t, 1H), 7.3-7.1 (m, 5H), 5.3 (m, 1H), 3.15 (dd, 1H), 2.84 (dd, 1H) ppm. MS (ESI) m/z (M+H)⁺ 314.9.

### N-(4-amino-3,4-dioxo-1-phenylbutan-2-yl)-2,3-dimethylbenzamide (26)

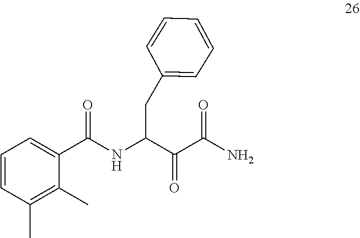 [SMILES: Cc1cccc(C(=O)NC(Cc2ccccc2)C(=O)C(N)=O)c1C]

Compound 26: ¹H NMR (400 MHz, DMSO): δ 8.68 (d, 1H), 8.12 (s, 1H), 7.85 (s, 1H), 7.34-6.9 (m, 8H), 5.33 (m, 1H), 3.16 (dd, 1H), 2.78 (dd, 1H), 2.21 (s, 3H), 2.02 (s, 3H) ppm. MS (ESI) m/z (M+H)⁺ 325.1.

### (S)—N-(4-amino-3,4-dioxo-1-phenylbutan-2-yl)-2-fluoro-6-iodobenzamide (27)

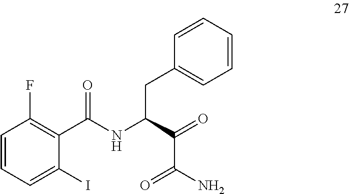 [SMILES: NC(=O)C(=O)[C@H](Cc1ccccc1)NC(=O)c1c(F)cccc1I]

Compound 27: ¹H NMR (400 MHz, DMSO): δ 9.11 (d, 1H), 8.09 (s, 1H), 7.81 (s, 1H), 7.6 (d, 1H), 7.3-7.1 (m, 7H), 5.44 (m, 1H), 3.1 (dd, 1H), 2.74 (dd, 1H) ppm. MS (ESI) m/z (M+H)⁺ 441.

### (S)—N-(4-amino-3,4-dioxo-1-phenylbutan-2-yl)-4-fluorobenzamide (28)

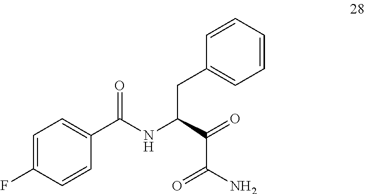 [SMILES: NC(=O)C(=O)[C@H](Cc1ccccc1)NC(=O)c1ccc(F)cc1]

Compound 28: ¹H NMR (400 MHz, DMSO): δ 8.89 (d, 1H), 8.09 (s, 1H), 7.9-7.7 (m, 3H), 7.4-7.1 (m, 7H), 5.34 (m, 1H), 3.2 (dd, 1H), 2.9 (dd, 1H) ppm. MS (ESI) m/z (M+H)⁺ 315.

### (S)—N-(4-amino-3,4-dioxo-1-phenylbutan-2-yl)-2-chloro-6-fluoro-3-methoxybenzamide (29)

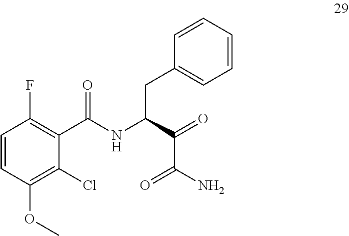 [SMILES: COc1ccc(F)c(C(=O)N[C@@H](Cc2ccccc2)C(=O)C(N)=O)c1Cl]

Compound 29: ¹H NMR (400 MHz, DMSO): δ 9.19 (d, 1H), 8.17 (s, 1H), 7.88 (s, 1H), 7.3-7.1 (m, 7H), 5.46 (m, 1H), 3.83 (s, 3H), 3.18 (dd, 1H), 2.76 (dd, 1H) ppm. MS (ESI) m/z (M+H)⁺ 379.4.

### (S)—N-(4-amino-3,4-dioxo-1-phenylbutan-2-yl)-2-chloro-6-fluoro-3-methylbenzamide (30)

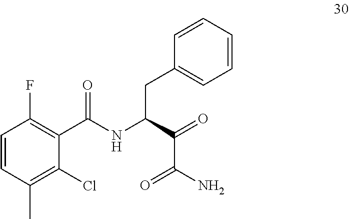 [SMILES: Cc1ccc(F)c(C(=O)N[C@@H](Cc2ccccc2)C(=O)C(N)=O)c1Cl]

Compound 30: ¹H NMR (400 MHz, DMSO): δ 9.18 (d, 1H), 8.17 (s, 1H), 7.88 (s, 1H), 7.45-7.1 (m, 7H), 5.47 (m, 1H), 3.83 (s, 3H), 3.18 (dd, 1H), 2.76 (dd, 1H), 2.27 (s, 3H) ppm. MS (ESI) m/z (M+H)⁺ 363.4.

### (S)—N-(4-amino-3,4-dioxo-1-phenylbutan-2-yl)-6-chloro-2-fluoro-3-methylbenzamide (31)

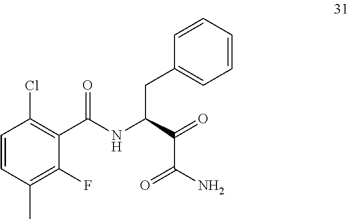 [SMILES: Cc1ccc(Cl)c(C(=O)N[C@@H](Cc2ccccc2)C(=O)C(N)=O)c1F]

Compound 31: ¹H NMR (400 MHz, DMSO): δ 9.18 (d, 1H), 8.15 (s, 1H), 7.88 (s, 1H), 7.45-7.1 (m, 7H), 5.46 (m, 1H), 3.83 (s, 3H), 3.18 (dd, 1H), 2.76 (dd, 1H), 2.2 (s, 3H) ppm. MS (ESI) m/z (M+H)⁺ 363.2.

### (S)—N-(4-amino-3,4-dioxo-1-phenylbutan-2-yl)-3-chloro-2-fluoro-6-(trifluoromethyl)benzamide (32)

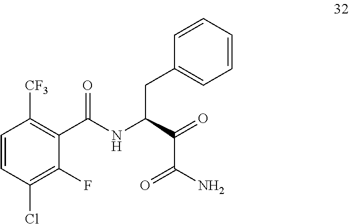 [SMILES: NC(=O)C(=O)[C@H](Cc1ccccc1)NC(=O)c1c(C(F)(F)F)ccc(Cl)c1F]

Compound 32: ¹H NMR (400 MHz, DMSO): δ 9.35 (d, 1H), 8.19 (s, 1H), 7.91 (s, 1H), 7.87 (d, 1H), 7.64 (d, 1H), 7.45-7.1 (m, 5H), 5.52 (m, 1H), 3.19 (dd, 1H), 2.77 (dd, 1H) ppm. MS (ESI) m/z (M+H)⁺ 417.3.

### (S)—N-(4-amino-3,4-dioxo-1-phenylbutan-2-yl)-2,4-dichloro-5-fluorobenzamide (33)

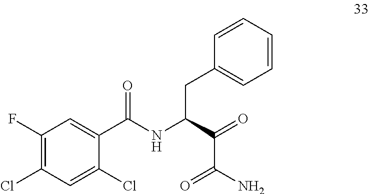 [SMILES: NC(=O)C(=O)[C@H](Cc1ccccc1)NC(=O)c1cc(F)c(Cl)cc1Cl]

Compound 33: ¹H NMR (400 MHz, DMSO): δ 9.05 (d, 1H), 8.14 (s, 1H), 7.88 (s, 1H), 7.87 (d, 1H), 7.35-7.2 (m, 6H), 5.36 (m, 1H), 3.83 (s, 3H), 3.21 (dd, 1H), 2.81 (dd, 1H) ppm. MS (ESI) m/z (M+H)⁺ 382.7.

### (S)—N-(4-amino-3,4-dioxo-1-phenylbutan-2-yl)-5-bromo-2-chlorobenzamide (34)

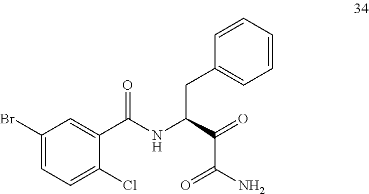 [SMILES: NC(=O)C(=O)[C@H](Cc1ccccc1)NC(=O)c1cc(Br)ccc1Cl]

Compound 34: ¹H NMR (400 MHz, DMSO): δ 9.05 (d, 1H), 8.14 (s, 1H), 7.88 (s, 1H), 7.64 (dd, 1H), 7.43 (d, 1H), 7.34-7.2 (m, 5H), 5.33 (m, 1H), 3.83 (s, 3H), 3.22 (dd, 1H), 2.8 (dd, 1H) ppm. MS (ESI) m/z (M+H)⁺ 409.2.

### (S)—N-(4-amino-3,4-dioxo-1-phenylbutan-2-yl)-5-bromo-2-methoxybenzamide (35)

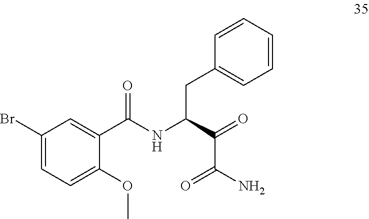 [SMILES: COc1ccc(Br)cc1C(=O)N[C@@H](Cc1ccccc1)C(=O)C(N)=O]

Compound 35: MS (ESI) m/z (M+H)⁺ 405.

### (S)—N-(4-amino-3,4-dioxo-1-phenylbutan-2-yl)-2-bromobenzamide (36)

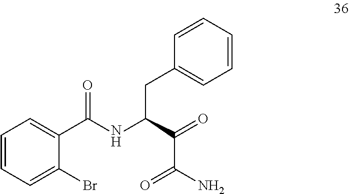 [SMILES: NC(=O)C(=O)[C@H](Cc1ccccc1)NC(=O)c1ccccc1Br]

Compound 36: ¹H NMR (400 MHz, DMSO): δ 8.93 (d, 1H), 8.13 (s, 1H), 7.87 (s, 1H), 7.61 (d, 1H), 7.41 (t, 1H), 7.4-7.1 (m, 7H), 5.36 (m, 1H), 3.19 (dd, 1H), 2.81 (dd, 1H) ppm. MS (ESI) m/z (M+H)⁺ 374.9.

### Example 15

### Compounds 37-48

### 5-chloro-2-methoxy-N-(1-oxo-3-phenylpropan-2-yl)benzamide (37)

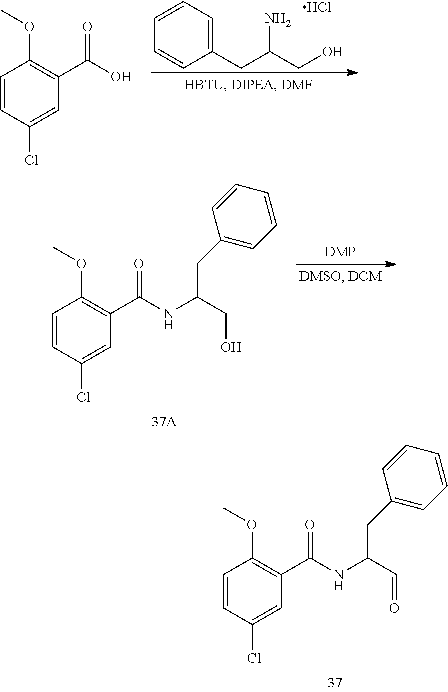

To a mixture of 5-chloro-2-methoxybenzoic acid (300 mg, 1.61 mmol) and 2-amino-3-phenylpropan-1-ol hydrochloride (362 mg, 1.93 mmol, HCl) in DMF (15 mL) was added HBTU (732 mg, 1.93 mmol) in one portion at 20° C. under N₂. The mixture was stirred at 20° C. for 0.1 h. Then to the mixture was added DIPEA (1.04 g, 8.04 mmol, 1.4 mL) and stirred at 20° C. for 0.5 h. The mixture was diluted with H₂O (50 mL) at 0° C. and stirred at 0° C. for 0.5 h, and the precipitate was formed, the solid was collected and was dried in vacuo to give compound 37A (450 mg, yield: 86.82%) as yellow solid. ¹H-NMR (400 MHz, DMSO-d₆) δ 8.11 (d, J=8.4 Hz, 1H), 7.60 (d, J=2.6 Hz, 1H), 7.50 (dd, J=2.6, 8.8 Hz, 1H), 7.31-7.24 (m, 4H), 7.21-7.14 (m, 2H), 4.12 (d, J=4.9 Hz, 1H), 3.85 (s, 3H), 3.06-2.86 (m, 2H), 2.69-2.69 (m, 1H), 2.84-2.68 (m, 1H). MS (ESI) m/z (M+H)⁺ 320.0.

To a mixture of compound 37A (150 mg, 469.07 umol) in DMSO (2 mL) and DCM (20 mL) was added DMP (597 mg, 1.41 mmol) in portion at 20° C. under N₂. The mixture was stirred at 20° C. for 0.5 h. The reaction mixture was diluted with DCM (20 mL), saturated NaHCO₃ (aqueous 30 mL) and Na₂S₂O₃ (aqueous 10%, 30 mL), then stirred for 15 min. Layers were separated. The organic layers were washed with water (150 mL×2) and brine (150 mL), dried over Na₂SO₄ and concentrated under reduced pressure to give a residue. The residue was triturated with EA (5 mL) and PE (25 mL), precipitate was formed, the solid was collected and was dried in vacuo to give compound 37 (75 mg, yield: 49.96%) as a yellow solid. ¹H-NMR (400 MHz, DMSO-d₆) δ 9.61 (s, 1H), 8.55 (d, J=6.8 Hz, 1H), 7.65 (d, J=2.9 Hz, 1H), 7.54 (dd, J=2.8, 8.9 Hz, 1H), 7.33-7.16 (m, 6H), 4.59 (dd, J=5.1, 6.9, 9.0 Hz, 1H), 3.81 (s, 3H), 3.22 (dd, J=4.9, 13.9 Hz, 1H), 3.02 (dd, J=9.0, 14.1 Hz, 1H). MS (ESI) m/z (M+H)⁺ 317.9.

### 3-chloro-2-fluoro-N-(1-oxo-3-phenylpropan-2-yl)-6-(trifluoromethyl)benzamide (38)

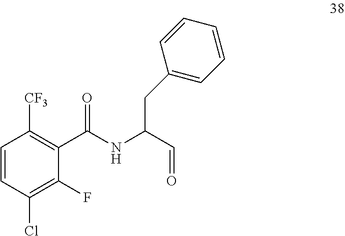 [SMILES: O=CC(Cc1ccccc1)NC(=O)c1c(C(F)(F)F)ccc(Cl)c1F]

Compound 38 was prepared following the procedure of compound 37 using the corresponding intermediate 2-amino-3-phenylpropan-1-ol hydrochloride and 3-chloro-2-fluoro-6-(trifluoromethyl)benzoic acid. Compound 38 (90 mg, yield 58.0%) was obtained as a light yellow solid ¹H NMR (DMSO-d₆, 400 MHz) δ 9.58 (s, 1H), 9.38 (br d, J=7.5 Hz, 1H), 7.92-7.88 (m, 1H), 7.67 (d, J=8.5 Hz, 1H), 7.33-7.27 (m, 4H), 7.24-7.20 (m, 1H), 4.65 (ddd, J=4.6, 7.4, 9.8 Hz, 1H), 3.25 (dd, J=4.4, 14.4 Hz, 1H), 2.83 (dd, J=9.9, 14.4 Hz, 1H). MS (ESI) m/z (M+H)⁺ 374.0.

### 2-fluoro-N-(1-oxo-3-phenylpropan-2-yl)-6-(trifluoromethyl)benzamide (39)

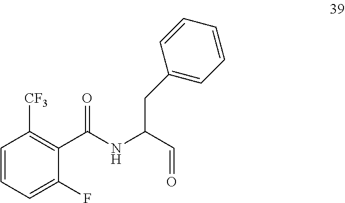 [SMILES: O=CC(Cc1ccccc1)NC(=O)c1c(F)cccc1C(F)(F)F]

Compound 39 was prepared following the procedure of compound 37 using the corresponding intermediate 2-amino-3-phenylpropan-1-ol hydrochloride and 2-fluoro-6-(trifluoromethyl)benzoic acid. Compound 39 (100 mg, yield 33.2%) was obtained as a light yellow solid ¹H NMR (400 MHz, CD₃CN) δ 9.63 (s, 1H), 7.67-7.55 (m, 2H), 7.45 (t, J=8.7 Hz, 1H), 7.34-7.21 (m, 5H), 4.71 (ddd, J=5.3, 7.4, 8.7 Hz, 1H), 3.28 (dd, J=5.1, 14.4 Hz, 1H), 2.99 (dd, J=8.7, 14.4 Hz, 1H). MS (ESI) m/z (M+H)⁺ 340.0.

### 2,6-difluoro-N-(1-oxo-3-phenylpropan-2-yl)benzamide (40)

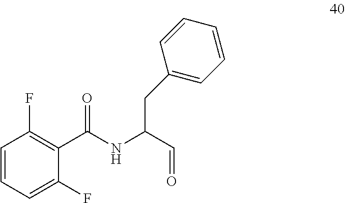 [SMILES: O=CC(Cc1ccccc1)NC(=O)c1c(F)cccc1F]

Compound 40 was prepared following the procedure of compound 37 using the corresponding intermediate 2-amino-3-phenylpropan-1-ol hydrochloride and 2,6-difluorobenzoic acid. Compound 40 (100 mg, yield 48.79%) was obtained as a white solid ¹H NMR (400 MHz, CD₃CN) δ 9.74-9.55 (m, 1H), 7.46 (tt, J=6.6, 8.5 Hz, 1H), 7.35-7.22 (m, 5H), 7.09-6.95 (m, 1H), 4.69 (ddd, J=4.9, 7.5, 9.0 Hz, 1H), 3.31 (dd, J=4.9, 14.3 Hz, 1H), 2.99 (dd, J=9.0, 14.3 Hz, 1H). MS (ESI) m/z (M+H)⁺ 289.9.

### 2-bromo-6-chloro-N-(1-oxo-3-phenylpropan-2-yl)benzamide (41)

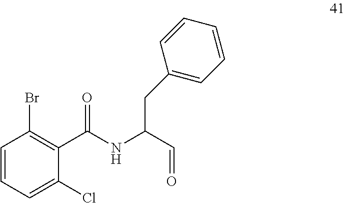 [SMILES: O=CC(Cc1ccccc1)NC(=O)c1c(Cl)cccc1Br]

Compound 41 was prepared following the procedure of compound 37 using the corresponding intermediate 2-amino-3-phenylpropan-1-ol hydrochloride and 2-bromo-6-chlorobenzoic acid. Compound 41 (30 mg, yield 15.9%) was obtained as a white solid. ¹H NMR (400 MHz, DMSO-d₆) δ 9.69 (s, 1H), 9.06 (br s, 1H), 7.61 (d, J=8.0 Hz, 1H), 7.54-7.38 (m, 1H), 7.38-7.19 (m, 6H), 4.72-4.54 (m, 1H), 3.26 (dd, J=4.5, 14.1 Hz, 1H), 2.93 (br dd, J=9.4, 14.7 Hz, 1H). MS (ESI) m/z (M+H)⁺ 367.0.

### 2-chloro-6-fluoro-3-methyl-N-(1-oxo-3-phenylpropan-2-yl)benzamide (42)

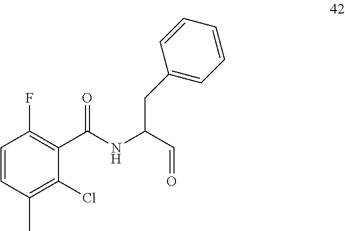 [SMILES: Cc1ccc(F)c(C(=O)NC(C=O)Cc2ccccc2)c1Cl]

Compound 42 was prepared following the procedure of compound 37 using the corresponding intermediate 2-amino-3-phenylpropan-1-ol hydrochloride and 2-chloro-6-fluoro-3-methylbenzoic acid. Compound 42 (80.6 mg, yield 24.13%) was obtained as a colorless oil. ¹H NMR (400 MHz, DMSO-d₆) δ 9.61 (s, 1H), 9.24 (d, J=7.5 Hz, 1H), 7.43 (ddd, J=0.7, 6.2, 8.6 Hz, 1H), 7.29 (d, J=4.6 Hz, 4H), 7.24-7.16 (m, 2H), 4.55 (ddd, J=4.4, 7.5, 10.1 Hz, 1H), 3.25 (dd, J=4.3, 14.2 Hz, 1H), 2.85 (dd, J=10.1, 14.3 Hz, 1H), 2.30 (s, 3H). MS (ESI) m/z (M+H)⁺ 320.1.

### 2-chloro-6-fluoro-3-methoxy-N-(1-oxo-3-phenylpropan-2-yl)benzamide (43)

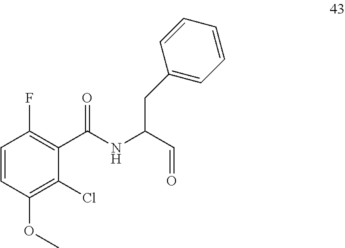 [SMILES: COc1ccc(F)c(C(=O)NC(C=O)Cc2ccccc2)c1Cl]

Compound 43 was prepared following the procedure of compound 37 using the corresponding intermediate 2-amino-3-phenylpropan-1-ol hydrochloride and 2-chloro-6-fluoro-3-methoxybenzoic acid. Compound 43 (125 mg, yield 38.19%) was obtained as a light yellow solid. ¹H NMR (400 MHz, DMSO-d₆) δ 9.61 (s, 1H), 9.25 (d, J=7.5 Hz, 1H), 7.29 (d, J=4.6 Hz, 4H), 7.27-7.19 (m, 3H), 4.54 (ddd, J=4.3, 7.4, 10.1 Hz, 1H), 3.84 (s, 3H), 3.25 (dd, J=4.4, 14.3 Hz, 1H), 2.84 (dd, J=10.1, 14.3 Hz, 1H). MS (ESI) m/z (M+H)⁺ 336.1.

### 2-chloro-N-(1-oxo-3-phenylpropan-2-yl)-1-naphthamide (44)

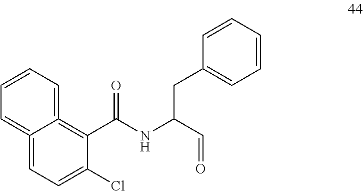

Compound 44 was prepared following the procedure of compound 37 using the corresponding intermediate 2-amino-3-phenylpropan-1-ol hydrochloride and 2-chloro-1-naphthoic acid. Compound 44 (65 mg, yield 41.70%) was obtained as a white solid. ¹H NMR (400 MHz, DMSO-d₆) δ 9.77 (s, 1H), 9.19 (d, J=7.9 Hz, 1H), 8.07-7.90 (m, 2H), 7.59-7.52 (m, 2H), 7.46 (br t, J=7.4 Hz, 1H), 7.38-7.26 (m, 6H), 4.88 (ddd, J=3.9, 7.6, 11.1 Hz, 1H), 3.40-3.36 (m, 1H), 2.82 (dd, J=11.2, 14.3 Hz, 1H). MS (ESI) m/z (M+H)⁺ 338.1.

### 2,6-dichloro-N-(1-OXO-3-phenylpropan-2-yl)benzamide (45)

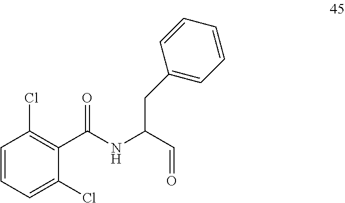 [SMILES: O=CC(Cc1ccccc1)NC(=O)c1c(Cl)cccc1Cl]

Compound 45 was prepared following the procedure of compound 37 using the corresponding intermediate 2-amino-3-phenylpropan-1-ol hydrochloride and 2,6-dichlorobenzoic acid. Compound 45 (150 mg, yield 45.47%) was obtained as a colorless oil. ¹H NMR (400 MHz, DMSO-d₆) δ 9.66 (s, 1H), 9.05 (br d, J=6.3 Hz, 1H), 7.50-7.37 (m, 3H), 7.34-7.18 (m, 5H), 4.61 (dt, J=4.9, 8.5 Hz, 1H), 3.26 (dd, J=4.8, 14.6 Hz, 1H), 2.91 (dd, J=9.7, 14.4 Hz, 1H). MS (ESI) m/z (M+H)⁺ 322.0.

### N-(1-oxo-3-phenylpropan-2-yl)dibenzo[b,d]furan-4-carboxamide (46)

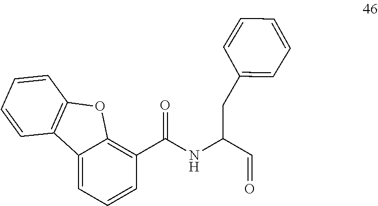 [SMILES: O=CC(Cc1ccccc1)NC(=O)c1cccc2c1oc1ccccc12]

Compound 46 was prepared following the procedure of compound 37 using the corresponding intermediate 2-amino-3-phenylpropan-1-ol hydrochloride and dibenzo[b,d]furan-4-carboxylic acid (7B). Compound 46 (90 mg, yield 28.10%) was obtained as a white solid. ¹H NMR (400 MHz, DMSO-d₆) δ 9.71 (s, 1H), 8.73 (d, J=7.1 Hz, 1H), 8.34 (dd, J=1.3, 7.7 Hz, 1H), 8.25-8.17 (m, 1H), 7.85 (dd, J=1.3, 7.7 Hz, 1H), 7.71 (d, J=8.2 Hz, 1H), 7.60 (ddd, J=1.3, 7.3, 8.4 Hz, 1H), 7.53-7.44 (m, 2H), 7.41-7.37 (m, 2H), 7.35-7.29 (m, 2H), 7.27-7.19 (m, 1H), 4.70 (ddd, J=4.7, 7.2, 9.5 Hz, 1H), 3.33-3.29 (m, 1H), 3.10 (dd, J=9.4, 14.0 Hz, 1H). MS (ESI) m/z (M+H)⁺ 344.1.

### 9-methyl-N-(1-oxo-3-phenylpropan-2-yl)-9H-carbazole-4-carboxamide (47)

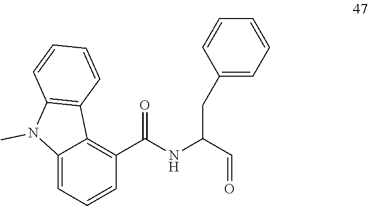 [SMILES: Cn1c2ccccc2c2c(C(=O)NC(C=O)Cc3ccccc3)cccc21]

Compound 47 was prepared following the procedure of compound 37 using the corresponding intermediate 2-amino-3-phenylpropan-1-ol hydrochloride and 9-methyl-9H-carbazole-4-carboxylic acid (11C). Compound 47 (55 mg, yield 43.0%) was obtained as a pale-yellow solid. ¹H NMR (400 MHz, DMSO-d₆) δ 9.84 (s, 1H), 8.14 (d, J=8.1 Hz, 1H), 7.65 (d, J=7.7 Hz, 1H), 7.56-7.47 (m, 3H), 7.39-7.23 (m, 7H), 7.16 (ddd, J=2.1, 5.9, 8.1 Hz, 1H), 4.83 (ddd, J=4.8, 7.7, 9.9 Hz, 1H), 3.90 (s, 3H), 3.46 (dd, J=4.8, 14.2 Hz, 1H), 3.08 (dd, J=9.9, 14.2 Hz, 1H). MS (ESI) m/z (M+H)⁺ 357.1.

### 9-methyl-N-(1-oxo-3-phenylpropan-2-yl)-9H-carbazole-4-carboxamide (48)

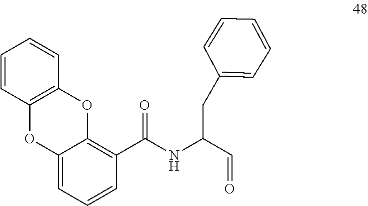 [SMILES: O=CC(Cc1ccccc1)NC(=O)c1cccc2c1Oc1ccccc1O2]

Compound 48 was prepared following the procedure of compound 37 using the corresponding intermediate 2-amino-3-phenylpropan-1-ol hydrochloride and dibenzo[b,e][1,4]dioxine-1-carboxylic acid (7B). Compound 48 (110 mg, yield 35.1%) was obtained as a white solid. ¹H NMR (400 MHz, DMSO-d₆) δ 9.62 (s, 1H), 8.61 (br, d, J=7.1 Hz, 1H), 7.27 (d, J=4.4 Hz, 4H), 7.20 (br, dd, J=4.3, 8.5 Hz, 1H), 7.12 (br, d, J=7.7 Hz, 1H), 7.09-7.04 (m, 1H), 7.02-6.94 (m, 4H), 6.74-6.69 (m, 1H), 4.64-4.56 (m, 1H), 3.27-3.19 (m, 1H), 2.97 (dd, J=9.6, 14.0 Hz, 1H). MS (ESI) m/z (M+H)⁺ 360.1.

### EXAMPLE SECTION II

### Example 1—Compounds 1, 12, 14, 18, 22, 28, 54, 94, 99, 100, 101, and 102 N-(4-amino-3,4-dioxo-1-phenylbutan-2-yl)-1-methyl-3-(quinolin-7-yl)-1H-pyrazole-4-carboxamide (1)

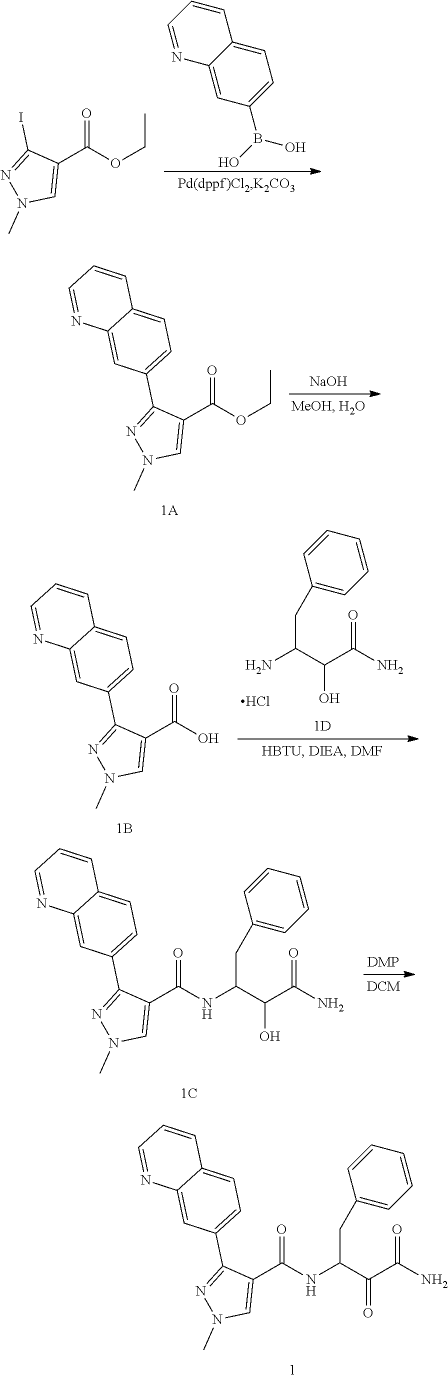

To a solution of ethyl 3-iodo-1-methyl-1H-pyrazole-4-carboxylate (0.5 g, 1.79 mmol) and 7-quinolylboronic acid (463 mg, 2.68 mmol) in dioxane (15 mL) and H₂O (1 mL) was added K₂CO₃ (494 mg, 3.57 mmol), then Pd(dppf)Cl₂ (261 mg, 357.06 umol) was added under N₂ atmosphere, the mixture was stirred at 80° C. for 17 h under N₂ atmosphere. The reaction mixture was concentrated to remove solvent, then diluted with EA (30 mL) and filtered, washed with EA (30 mL×2), the filtrate was concentrated to give a residue. The residue was purified by flash silica gel chromatography (ISCO®; 4 g SepaFlash® Silica Flash Column, Eluent of 0-70% Ethyl acetate/Petroleum ether gradient @ 20 mL/min). Compound 1A (0.48 g, yield: 91.4%) as yellow oil was obtained. ¹H NMR (400 MHz, CDCl₃) δ 8.94 (dd, J=1.8, 4.2 Hz, 1H), 8.56-8.49 (m, 1H), 8.18 (d, J=8.6 Hz, 1H), 8.04-7.94 (m, 2H), 7.85 (d, J=8.4 Hz, 1H), 7.44-7.37 (m, 1H), 4.26 (q, J=7.1 Hz, 2H), 4.01 (s, 3H), 1.30-1.24 (m, 3H). MS (ESI) m/z (M+H)⁺ 282.2.

To a solution of compound 1A (0.48 g, 1.71 mmol) in MeOH (10 mL) was added the solution of NaOH (341 mg, 8.53 mmol) in H₂O (2 mL), the mixture was stirred at 50° C. for 18 h. The reaction mixture was concentrated to remove MeOH, diluted with water (10 mL), extracted with EA (20 mL), the aqueous phase was acidized with 1N HCl to pH ˜3, the precipitate was formed, the solid was filtered and lyophilized. Compound 1B (0.22 g, yield: 50.9%) as yellow solid was obtained, which was used into the next step without further purification. ¹H NMR (400 MHz, DMSO-d₆) δ 9.10 (dd, J=1.4, 4.7 Hz, 1H), 8.77 (d, J=7.9 Hz, 1H), 8.65 (s, 1H), 8.42 (s, 1H), 8.23-8.11 (m, 2H), 7.81 (dd, J=4.6, 8.4 Hz, 1H), 3.97 (s, 3H). MS (ESI) m/z (M+H)⁺ 254.2.

To a mixture of compound 1B (210 mg, 829.20 umol), Intermediate 1D (230 mg, 997.01 umol, HCl) in DMF (6 mL) was added DIEA (4.13 mmol, 720 uL), and then added HBTU (377 mg, 994.09 umol). The mixture was stirred at 25° C. for 1.5 h. The reaction mixture was added in H₂O (40 mL, 0° C.), a quantity of yellow precipitate was formed, and then stirred at 0° C. for 15 min. The solid were washed with H₂O (10 mL×2) and lyophilized. The residue was triturated in DCM (3 mL) and PE (20 mL), and then filtered. Compound 1C (190 mg, yield: 50.8%) was obtained as a yellow solid. ¹H NMR (400 MHz, DMSO-d₆) δ 8.90 (s, 1H), 8.39-8.30 (m, 2H), 8.19-8.07 (m, 1H), 7.95-7.83 (m, 2H), 7.81-7.72 (m, 1H), 7.56-7.46 (m, 1H), 7.41-7.11 (m, 7H), 5.92-5.74 (m, 1H), 4.58-4.41 (m, 1H), 4.12-4.03 (m, 1H), 3.93 (s, 3H), 3.85 (br d, J=4.3 Hz, 1H), 3.19-2.74 (m, 2H). MS (ESI) m/z (M+H)⁺ 430.2.

To a solution of compound 1C (0.19 g, 442.41 umol) in DMSO (10 mL) and DCM (60 mL) was added DMP (751 mg, 1.77 mmol), the mixture was stirred at 25° C. for 1.5 h. The reaction mixture was diluted with DCM (20 mL), then quenched with saturated Na₂S₂O₃ (60 mL) and saturated NaHCO₃ (60 mL), extracted with DCM (50 mL×2), the organic layers were washed with water (100 mL×2) and brine (100 mL×2), dried over Na₂SO₄, filtered and concentrated to give a residue. The residue was triturated in CH₃CN (3 mL) and isopropyl ether (3 mL), then filtered and lyophilized. Compound 1 (30 mg, yield: 15.5%) as light yellow solid was obtained. ¹H NMR (400 MHz, DMSO-d₆) δ 8.89 (br s, 1H), 8.42-8.26 (m, 2H), 8.12 (br s, 1H), 8.00-7.43 (m, 5H), 7.33-6.76 (m, 6H), 5.43-4.51 (m, 1H), 3.94 (s, 3H), 3.21 (d, J=14.1 Hz, 1H), 2.96-2.84 (m, 1H). MS (ESI) m/z (M+H)⁺ 428.1.

### N-(4-amino-3,4-dioxo-1-phenylbutan-2-yl)-3-(2,3-dimethoxyphenyl)-1-methyl-1H-pyrazole-4-carboxamide (12)

Compounds 12, 14, 18, 22, 28, 54, 94, 99, 100, 101, and 102 were prepared as in Example 1 using the corresponding boronic acid or boronate ester, respectively. Compound 12 (88 mg, yield: 66.5%) as a light yellow solid was obtained: ¹H NMR (400 MHz, DMSO-d₆) δ 8.15 (s, 1H), 8.02 (s, 1H), 7.83-7.73 (m, 2H), 7.30-7.11 (m, 5H), 7.09-6.98 (m, 2H), 6.72 (dd, J=1.5, 7.3 Hz, 1H), 5.42-5.15 (m, 1H), 3.88 (s, 3H), 3.81 (s, 3H), 3.42 (s, 3H), 3.10 (dd, J=3.5, 14.1 Hz, 1H), 2.74 (dd, J=9.5, 13.6 Hz, 1H). MS (ESI) m/z (M+H)⁺ 437.2.

### N-(4-amino-3,4-dioxo-1-phenylbutan-2-yl)-1-methyl-3-(quinolin-8-yl)-1H-pyrazole-4-carboxamide (14)

Compound 14 (90 mg, yield: 53.7%) as a white solid was obtained: ¹H NMR (400 MHz, DMSO-d₆) δ 8.64 (dd, J=1.9, 4.1 Hz, 1H), 8.36 (dd, J=1.8, 8.4 Hz, 1H), 8.16 (s, 1H), 7.99 (dd, J=1.5, 8.2 Hz, 1H), 7.89 (s, 1H), 7.82 (d, J=7.5 Hz, 1H), 7.69 (s, 1H), 7.65-7.55 (m, 2H), 7.47 (dd, J=4.1, 8.3 Hz, 1H), 7.19-7.11 (m, 3H), 6.92 (dd, J=2.0, 7.3 Hz, 2H), 5.13-5.05 (m, 1H), 3.94-3.85 (m, 3H), 2.94 (dd, J=4.0, 13.9 Hz, 1H), 2.59-2.50 (m, 1H). MS (ESI) m/z (M+H)⁺ 428.2.

### N-(4-amino-3,4-dioxo-1-phenylbutan-2-yl)-1-(difluoromethyl)-3-(quinolin-8-yl)-1H-pyrazole-4-carboxamide (18)

Compound 18 (80 mg, yield: 54.7%) as a white solid was obtained: ¹H NMR (400 MHz, DMSO-d₆) δ 8.67-8.60 (m, 1H), 8.56 (dd, J=1.8, 4.2 Hz, 1H), 8.42 (d, J=7.5 Hz, 1H), 8.38-8.33 (m, 1H), 8.03 (dd, J=1.3, 8.4 Hz, 1H), 7.95-7.77 (m, 2H), 7.76-7.69 (m, 2H), 7.65-7.59 (m, 1H), 7.46 (dd, J=4.2, 8.4 Hz, 1H), 7.26-7.16 (m, 3H), 7.10 (d, J=6.8 Hz, 2H), 5.22-5.05 (m, 1H), 3.02 (dd, J=3.6, 14.0 Hz, 1H), 2.64 (dd, J=9.7, 13.9 Hz, 1H). MS (ESI) m/z (M+H)⁺ 464.1.

### N-(4-amino-3,4-dioxo-1-phenylbutan-2-yl)-1-(difluoromethyl)-3-(isoquinolin-8-yl)-1H-pyrazole-4-carboxamide (22)

Compound 22 (90 mg, yield: 53.1%) as a white solid was obtained: ¹H NMR (400 MHz, DMSO-d₆) δ 9.14-9.06 (m, 1H), 8.81 (s, 1H), 8.51 (d, J=5.5 Hz, 1H), 8.29 (br s, 1H), 8.09-7.79 (m, 3H), 7.76 (t, J=7.8 Hz, 1H), 7.70 (d, J=9.0 Hz, 1H), 7.57 (d, J=7.0 Hz, 1H), 7.51 (br s, 1H), 7.25-7.12 (m, 5H), 5.36-5.07 (m, 1H), 3.16 (d, J=4.5 Hz, 1H), 2.83 (dd, J=9.2, 13.9 Hz, 1H). MS (ESI) m/z (M+H)⁺ 464.1.

### N-(4-amino-3,4-dioxo-1-phenylbutan-2-yl)-1-methyl-3-(2-methylfuran-3-yl)-1H-pyrazole-4-carboxamide (28)

Compound 28 (170 mg, yield: 85.5%) as a white solid was obtained: ¹H NMR (400 MHz, DMSO-d₆) δ 8.12-7.99 (m, 3H), 7.77 (s, 1H), 7.40 (d, J=2.0 Hz, 1H), 7.29-7.15 (m, 5H), 6.48 (d, J=1.8 Hz, 1H), 5.38-5.13 (m, 1H), 3.83 (s, 3H), 3.12 (dd, J=3.9, 13.8 Hz, 1H), 2.79 (dd, J=9.7, 13.9 Hz, 1H), 2.19 (s, 3H). MS (ESI) m/z (M+H)⁺381.1.

### N-(4-amino-3,4-dioxo-1-phenylbutan-2-yl)-3-(isoquinolin-8-yl)-1-methyl-1H-pyrazole-4-carboxamide (54)

Compound 54 (15 mg, yield: 14.5%) as a white solid was obtained: ¹H NMR (400 MHz, DMSO-d₆) δ 9.17-9.03 (m, 1H), 8.44 (d, J=6.0 Hz, 1H), 8.35 (d, J=7.5 Hz, 1H), 7.94-7.92 (m, 1H), 7.82 (d, J=5.7 Hz, 1H), 7.82-7.79 (m, 1H), 7.74-7.61 (m, 2H), 7.46-7.28 (m, 2H), 7.26-6.97 (m, 6H), 5.16-5.11 (m, 0.5H), 4.47-4.31 (m, 0.5H), 3.99-3.92 (m, 3H), 3.19-2.70 (m, 2H). MS (ESI) m/z (M+H)⁺ 428.1.

### N-(4-amino-3,4-dioxo-1-phenylbutan-2-yl)-2-(difluoromethyl)-4-(1H-indazol-7-yl)oxazole-5-carboxamide (94)

Intermediate derivatives 7-(4,4,5,5-tetramethyl-1,3,2-dioxaborolan-2-yl)-1-((2-(trimethylsilyl)ethoxy)methyl)-1H-indazole and ethyl 1-(difluoromethyl)-3-iodo-1H-pyrazole-4-carboxylate were subjected to conditions as described for compound 12 to yield compound 94. Compound 94 (63 mg, yield: 40.9%) as a pale-yellow solid was obtained: ¹H NMR (400 MHz, DMSO-d₆) δ 12.94 (br s, 1H), 8.91 (d, J=7.5 Hz, 1H), 8.63 (s, 1H), 8.19-8.12 (m, 2H), 8.01-7.84 (m, 2H), 7.81 (d, J=7.8 Hz, 1H), 7.75 (d, J=7.3 Hz, 1H), 7.31 (d, J=4.3 Hz, 4H), 7.26-7.22 (m, 1H), 7.10 (t, J=7.7 Hz, 1H), 5.42-5.34 (m, 1H), 3.21 (dd, J=3.9, 13.9 Hz, 1H), 2.85 (dd, J=9.9, 13.9 Hz, 1H). MS (ESI) m/z (M+H)⁺=453.1.

### N-(4-amino-3,4-dioxo-1-phenylbutan-2-yl)-1-methyl-3-(2-methyl-2H-indazol-7-yl)-1H-pyrazole-4-carboxamide (99)

Intermediate derivatives (2-methyl-2H-indazol-7-yl)boronic acid and ethyl 3-iodo-1-methyl-1H-pyrazole-4-carboxylate were subjected to conditions as described for compound 12 to yield compound 99. Compound 99 (70 mg, yield: 23.4%) as a white solid was obtained: ¹H NMR (400 MHz, DMSO-d₆) δ 8.41 (s, 1H), 8.15 (s, 1H), 8.00 (s, 1H), 7.93 (d, J=7.6 Hz, 1H), 7.80-7.74 (m, 2H), 7.18-7.05 (m, 5H), 6.82-6.78 (m, 2H), 5.25-5.18 (m, 1H), 4.09 (s, 3H), 3.92-3.87 (m, 3H), 3.01-2.95 (m, 1H), 2.47-2.41 (m, 1H). MS (ESI) m/z (M+H)⁺ 431.1.

### N-(4-amino-3,4-dioxo-1-phenylbutan-2-yl)-3-(1-isopropyl-1H-indazol-4-yl)-1-methyl-1H-pyrazole-4-carboxamide (100)

Intermediate derivatives 1-isopropyl-4-(4,4,5,5-tetramethyl-1,3,2-dioxaborolan-2-yl)-1H-indazole and ethyl 1-(difluoromethyl)-3-iodo-1H-pyrazole-4-carboxylate were subjected to conditions as described for compound 12 to yield compound 100. Compound 100 (60 mg, yield: 48.41%) as a white solid was obtained. MS (ESI) m/z (M+H)⁺=459.2. ¹H NMR (400 MHz, DMSO-d₆) δ 8.31 (d, J=7.2 Hz, 1H), 8.09-8.05 (m, 2H), 8.04 (br. s, 1H), 7.79 (br. s, 1H), 7.60 (d, J=7.2 Hz, 1H), 7.30-7.14 (m, 7H), 5.31-5.20 (m, 1H), 5.03-4.91 (m, 1H), 3.92 (s, 3H), 3.16-3.04 (m, 1H), 2.83-2.71 (m, 1H), 1.45 (d, J=6.4 Hz, 6H).

### N-(4-amino-3,4-dioxo-1-phenylbutan-2-yl)-3-(benzo[b]thiophen-7-yl)-1-methyl-1H-pyrazole-4-carboxamide (101)

Compound 101 (50 mg, yield: 11.58%) as a white solid was obtained: ¹H NMR (400 MHz, DMSO-d₆) δ 7.96 (s, 1H), 7.92 (dd, J=1.1, 7.9 Hz, 1H), 7.62 (d, J=5.5 Hz, 1H), 7.51-7.47 (m, 2H), 7.44-7.38 (m, 1H), 7.23-7.19 (m, 3H), 7.00 (dd, J=2.9, 6.7 Hz, 2H), 6.97-6.92 (m, 1H), 6.58 (br d, J=6.8 Hz, 1H), 6.20 (br s, 1H), 5.37 (ddd, J=4.8, 7.0, 8.5 Hz, 1H), 3.99-3.93 (m, 3H), 3.17 (dd, J=4.9, 13.9 Hz, 1H), 2.81 (dd, J=8.7, 13.9 Hz, 1H). MS (ESI) m/z (M+H)⁺ 433.1.

### N-(4-amino-3,4-dioxo-1-phenylbutan-2-yl)-3-(benzo[b]thiophen-4-yl)-1-methyl-1H-pyrazole-4-carboxamide (102)

Compound 102 (100 mg, yield: 71.0%) as a white solid was obtained: ¹H NMR (DMSO-d₆, 400 MHz): δ 8.19 (s, 1H), 8.15 (d, J=7.5 Hz, 1H), 8.02 (s, 1H), 8.00-7.94 (m, 1H), 7.79 (s, 1H), 7.67 (d, J=5.5 Hz, 1H), 7.37-7.15 (m, 8H), 5.31-5.14 (m, 1H), 3.95 (s, 3H), 3.11 (dd, J=3.8, 13.8 Hz, 1H), 2.77 (dd, J=9.7, 13.9 Hz, 1H). MS (ESI) m/z (M+H)⁺ 433.1.

### Example 2—Compounds 4, 10, 13, 25, 37, 49, and 63

### N-(4-amino-3,4-dioxo-1-phenylbutan-2-yl)-1-(difluoromethyl)-3-(isoquinolin-1-yl)-1H-pyrazole-4-carboxamide (3)

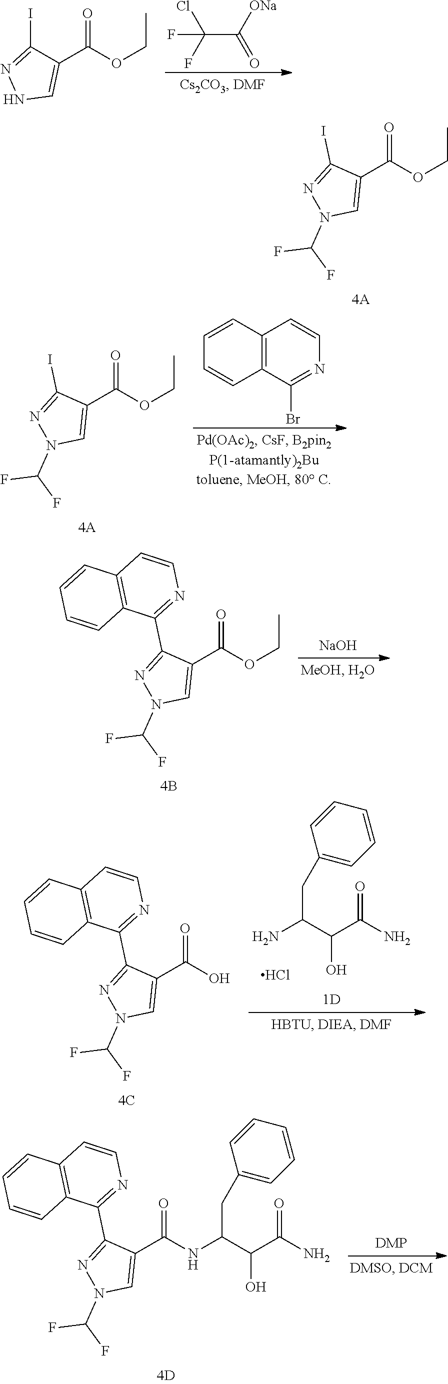

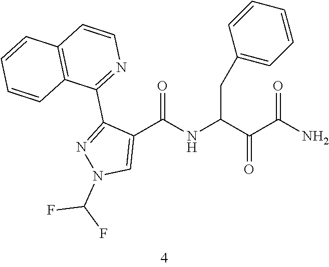 [SMILES: NC(=O)C(=O)C(Cc1ccccc1)NC(=O)c1cn(C(F)F)nc1-c1nccc2ccccc12]

To a solution of ethyl 3-iodo-1H-pyrazole-4-carboxylate (20 g, 75.18 mmol) in DMF (100 mL) was added sodium 2-chloro-2,2-difluoroacetate (22.92 g, 150.36 mmol) and Cs₂CO₃ (48.99 g, 150.36 mmol). The mixture was stirred at 100° C. for 16 h. The reaction mixture was concentrated, the residue was diluted with H₂O (200 mL) and extracted with EtOAc (100 mL×3). The combined organic layers were washed with brine (200 mL), dried over Na₂SO₄, filtered and concentrated under reduced pressure to give a residue. The residue was purified by flash silica gel chromatography (ISCO®; X g SepaFlash® Silica Flash Column, eluent of 0%˜10%˜20% Ethyl acetate/Petroleum ether gradient). Compound 4A (9.1 g, yield: 38.30%) was obtained as a white solid. ¹H NMR (400 MHz, CDCl₃) δ 8.47-7.95 (m, 1H), 7.44-6.95 (m, 1H), 4.53-4.17 (m, 2H), 1.54-1.17 (m, 3H).

To a solution of compound 4A (500 mg, 1.58 mmol), 1-bromoisoquinoline (329 mg, 1.58 mmol), CsF (480 mg, 3.16 mmol), and B₂pin₂ (603 mg, 2.37 mmol) in toluene (8 mL) and MeOH (8 mL) was added Pd(OAc)₂ (35.52 mg, 158.21 umol) and P(1-adamantyl)₂Bu (57 mg, 158.98 umol) in one portion under N₂ atmosphere. The mixture was stirred at 80° C. for 16 hr under N₂ atmosphere. The reaction mixture was filtered and concentrated, the residue was diluted with H₂O (10 mL) and extracted with EA (10 mL×3). The organic layers were dried over Na₂SO₄, filtered and concentrated to give a residue. The residue was purified by flash silica gel chromatography (PE:EA=5:1 to 2:1). Compound 4B (80 mg, yield: 12.1%) was obtained as a yellow solid. ¹H NMR (400 MHz, CDCl₃) δ 8.64 (d, J=5.7 Hz, 1H), 8.54 (s, 1H), 7.90 (d, J=8.2 Hz, 1H), 7.83-7.75 (m, 2H), 7.74-7.68 (m, 1H), 7.55 (ddd, J=1.1, 7.0, 8.4 Hz, 1H), 7.48-7.29 (m, 1H), 4.01 (q, J=7.1 Hz, 2H), 0.86 (t, J=7.2 Hz, 3H). MS (ESI) m/z (M+H)⁺ 317.9.

To a solution of compound 4B (80 mg, 252.14 umol) in MeOH (10 mL) and H₂O (3 mL) was added NaOH (40 mg, 1.00 mmol). The mixture was stirred at 50° C. for 16 hr. The reaction mixture was concentrated, diluted with water (10 mL), extracted with MTBE (10 mL), then the aqueous phase were acidized with 2N HCl to pH ˜2-3, and lyophilized. Then the residue was stirred in the solution (DCM:MeOH=10:1), filtered and concentrated to give a residue. Compound 4C (39 mg, yield: 53.5%) was obtained as a brown solid. ¹H NMR (400 MHz, DMSO-d₆) δ 8.91 (s, 1H), 8.51 (d, J=5.7 Hz, 1H), 8.01 (t, J=8.5 Hz, 2H), 7.98-7.85 (m, 2H), 7.81-7.72 (m, 1H), 7.62 (t, J=7.7 Hz, 1H).

To a solution of compound 4C (64 mg, 221.27 umol) and Intermediate 1D (56 mg, 242.75 umol, HCl) in DMF (10 mL) was added HBTU (101 mg, 266.32 umol), then was added DIEA (114 mg, 882.06 umol, 153.64 uL) and stirred at 25° C. for 2 hr. The reaction mixture was diluted with water (40 mL), extracted with EA (30 mL×3), the organic layers were concentrated to give a residue. The residue was triturated in PE:EA (10:1, 20 mL) and collected by filtration. Compound 4D (80 mg, yield: 76.8%) was obtained as a pale-yellow solid. ¹H NMR (400 MHz, DMSO-d₆) δ 9.71-9.27 (m, 1H), 8.84-8.54 (m, 2H), 8.41-7.57 (m, 6H), 7.30 (br s, 1H), 7.16-6.62 (m, 6H), 6.17-5.76 (m, 1H), 4.52-4.23 (m, 1H), 3.93-3.75 (m, 1H), 2.85-2.67 (m, 2H). MS (ESI) m/z (M+H)⁺466.1.

To a solution of compound 4D (80 mg, 171.88 umol) in DMSO (10 mL) and DCM (50 mL) was added DMP (292 mg, 688.45 umol). The mixture was stirred at 25° C. for 3 hr. The reaction mixture was diluted with DCM (20 mL), quenched with saturated NaHCO₃ (25 mL) and saturated Na₂S₂O₃ (25 mL), the mixture was stirred 10 min. The organic layer was washed with water (40 mL×2), brine (40 mL×2), dried over Na₂SO₄, then filtered and concentrated to give a residue. The residue was purified by flash silica gel chromatography (PE:EA=1:1 to 0:1). Compound 4 (25 mg, yield: 29.9%) was obtained as a pale yellow solid. ¹H NMR (400 MHz, DMSO-d₆) δ 9.81 (d, J=7.3 Hz, 1H), 8.88 (s, 1H), 8.37 (d, J=5.5 Hz, 1H), 8.28 (d, J=9.0 Hz, 1H), 8.14-7.97 (m, 3H), 7.92 (d, J=6.0 Hz, 1H), 7.83 (br d, J=5.3 Hz, 2H), 7.72-7.66 (m, 1H), 7.06-6.92 (m, 5H), 5.46-5.36 (m, 1H), 3.15 (br dd, J=4.5, 14.0 Hz, 1H), 2.88 (dd, J=8.7, 14.0 Hz, 1H). MS (ESI) m/z (M+H)⁺464.1.

### N-(4-amino-3,4-dioxo-1-phenylbutan-2-yl)-3-(isoquinolin-1-yl)-1-methyl-1H-pyrazole-4-carboxamide (10)

Compounds 10, 13, 25, 37, 49, and 63 were prepared as in Example 2 using the corresponding carboxylic acid, respectively. Ethyl 3-iodo-1-methyl-1H-pyrazole-4-carboxylate was used to obtain compound 10 (55 mg, yield: 61.2%) as a pale yellow solid was obtained: ¹H NMR (400 MHz, DMSO-d₆) δ 10.21 (d, J=7.3 Hz, 1H), 8.61 (d, J=8.2 Hz, 1H), 8.37 (s, 1H), 8.32 (d, J=6.0 Hz, 1H), 8.12-8.02 (m, 2H), 7.90-7.80 (m, 3H), 7.69 (t, J=7.8 Hz, 1H), 7.05-6.88 (m, 5H), 5.47 (d, J=4.9 Hz, 1H), 4.01 (s, 3H), 3.17 (dd, J=4.7, 13.8 Hz, 1H), 2.91 (dd, J=7.3, 14.3 Hz, 1H). MS (ESI) m/z (M+H)⁺ 428.2.

### N-(4-amino-3,4-dioxo-1-phenylbutan-2-yl)-1-methyl-3-(quinoxalin-2-yl)-1H-pyrazole-4-carboxamide (13)

Ethyl 3-iodo-1-methyl-1H-pyrazole-4-carboxylate was used to obtain compound 13 (20 mg, yield: 76.2%) as a white solid was obtained: ¹H NMR (400 MHz, DMSO-d₆) δ 11.18 (d, J=8.2 Hz, 1H), 9.60 (s, 1H), 8.46 (s, 1H), 8.19 (s, 1H), 8.12 (d, J=8.2 Hz, 1H), 7.92-7.84 (m, 2H), 7.77 (dt, J=1.3, 7.7 Hz, 1H), 7.65 (d, J=8.4 Hz, 1H), 7.01-6.93 (m, 4H), 6.90-6.79 (m, 1H), 5.79-5.74 (m, 1H), 4.03 (s, 3H), 3.29-3.18 (m, 2H). MS (ESI) m/z (M+H)⁺ 429.1.

### N-(4-amino-3,4-dioxo-1-phenylbutan-2-yl)-1-(difluoromethyl)-3-(quinoxalin-2-yl)-1H-pyrazole-4-carboxamide (25)

Compound 25 (20 mg, yield: 52.2%) as a white solid was obtained: ¹H NMR (400 MHz, DMSO-d₆) δ 10.80 (d, J=8.2 Hz, 1H), 9.51 (s, 1H), 8.92 (s, 1H), 8.23-7.82 (m, 5H), 7.78 (dt, J=1.3, 7.6 Hz, 1H), 7.71-7.65 (m, 1H), 7.01-6.89 (m, 4H), 6.88-6.82 (m, 1H), 5.77-5.67 (m, 1H), 3.24-3.12 (m, 2H). MS (ESI) m/z (M+H)⁺ 465.1.

### N-(4-amino-3,4-dioxo-1-phenylbutan-2-yl)-3-(6,7-dimethoxyquinolin-4-yl)-1-methyl-1H-pyrazole-4-carboxamide (37)

Compound 37 (15 mg, yield: 47.2%) as a pale yellow solid was obtained: ¹H NMR (400 MHz, DMSO-d₆) δ 8.62 (d, J=4.5 Hz, 1H), 8.35-8.23 (m, 1H), 7.71 (br d, J=6.8 Hz, 1H), 7.65 (br s, 1H), 7.49 (br s, 1H), 7.41 (s, 1H), 7.26-7.17 (m, 5H), 7.10 (d, J=6.8 Hz, 2H), 5.27-5.18 (m, 1H), 3.99 (s, 3H), 3.96 (s, 3H), 3.72 (s, 3H), 3.16-3.21 (m, 1H), 2.75-2.81 (m, 1H). MS (ESI) m/z (M+H)⁺ 488.2.

### N-(4-amino-3,4-dioxo-1-phenylbutan-2-yl)-1-methyl-3-(quinazolin-4-yl)-1H-pyrazole-4-carboxamide (49)

Ethyl 3-iodo-1-methyl-1H-pyrazole-4-carboxylate was used to obtain compound 49 (62 mg, yield: 61.3%) as a white solid was obtained: ¹H NMR (400 MHz, DMSO-d₆) δ 10.10 (d, J=7.5 Hz, 1H), 8.97 (s, 1H), 8.66 (d, J=8.4 Hz, 1H), 8.44 (s, 1H), 8.11 (s, 1H), 8.06 (d, J=3.5 Hz, 2H), 7.84 (s, 1H), 7.80-7.72 (m, 1H), 7.01 (s, 5H), 5.61-5.35 (m, 1H), 4.03 (s, 3H), 3.18 (dd, J=5.0, 14.2 Hz, 1H), 2.99 (dd, J=7.6, 14.0 Hz, 1H). MS (ESI) m/z (M+H)⁺ 429.1.

### N-(4-amino-3,4-dioxo-1-phenylbutan-2-yl)-1-(difluoromethyl)-3-(quinazolin-4-yl)-1H-pyrazole-4-carboxamide (63)

Compound 63 (28 mg, yield: 73.3%) as a pale yellow solid was obtained: ¹H NMR (400 MHz, DMSO-d₆) δ 9.51 (d, J=7.5 Hz, 1H), 9.11 (s, 1H), 8.92 (s, 1H), 8.23 (d, J=8.6 Hz, 1H), 8.18-7.99 (m, 4H), 7.90-7.80 (m, 1H), 7.79-7.71 (m, 1H), 7.14-7.03 (m, 5H), 5.36 (dt, J=4.6, 7.9 Hz, 1H), 3.14 (dd, J=4.2, 13.9 Hz, 1H), 2.89 (dd, J=8.5, 14.0 Hz, 1H). MS (ESI) m/z (M+H)⁺ 465.1.

### Example 3

### N-(4-amino-3,4-dioxo-1-phenylbutan-2-yl)-1-methyl-3-(piperazin-1-yl)-1H-pyrazole-4-carboxamide Hydrochloride (2)

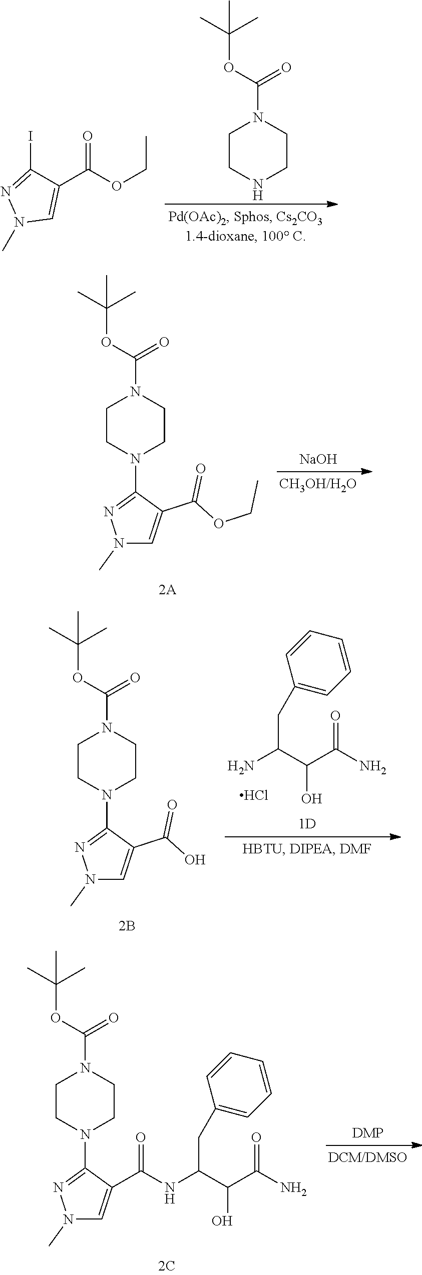

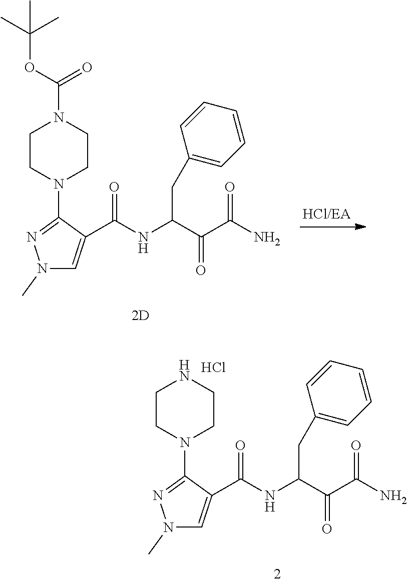 [SMILES: Cn1cc(C(=O)NC(Cc2ccccc2)C(=O)C(N)=O)c(N2CCN(C(=O)OC(C)(C)C)CC2)n1 ; Cn1cc(C(=O)NC(Cc2ccccc2)C(=O)C(N)=O)c(N2CCNCC2)n1 ; Cl]

To a solution of ethyl 3-iodo-1-methyl-1H-pyrazole-4-carboxylate (0.5 g, 1.79 mmol) and tert-butyl piperazine-1-carboxylate (665 mg, 3.57 mmol) in dioxane (20 mL) was added S-Phos (147 mg, 357.06 umol) and Cs₂CO₃ (1.16 g, 3.57 mmol), then Pd(OAc)₂ (40 mg, 178.53 umol) was added under N₂ atmosphere. The reaction was stirred at 100° C. for 17 h. The reaction mixture was filtered, washed with EA (30 mL×2), the filtrate was concentrated to give a residue. The residue was purified by flash silica gel chromatography (ISCO®; 4 g SepaFlash® Silica Flash Column, Eluent of 0˜10% Ethyl acetate/Petroleum ethergradient @ 20 mL/min). Compound 2A (0.15 g, yield: 22.8%) as light yellow oil was obtained. ¹H NMR (400 MHz, CDCl₃) δ 7.75 (s, 1H), 4.24 (q, J=7.1 Hz, 2H), 3.76 (s, 3H), 3.61-3.54 (m, 4H), 3.30-3.20 (m, 4H), 1.47 (s, 9H), 1.32 (t, J=7.1 Hz, 3H). MS (ESI) m/z (M+H)⁺ 339.1.

Compound 2A was transformed into compound 2D as shown in Example 1. Compound 2D (0.10 g, yield: 72.2%) as yellow solid was obtained. ¹H NMR (400 MHz, DMSO-d₆) δ 8.30-7.84 (m, 4H), 7.30-7.17 (m, 3H), 7.07 (d, J=7.1 Hz, 2H), 5.57-5.44 (m, 1H), 3.76-3.67 (m, 3H), 3.28-3.08 (m, 6H), 2.86-2.70 (m, 4H), 1.43-1.38 (m, 9H). MS (ESI) m/z (M+H)⁺ 485.3.

To a solution of compound 2D (100 mg, 206.38 umol) in EtOAc (2 mL) was added HCl/EtOAc (4M, 4 mL), the mixture was stirred at 25° C. for 4 h. The reaction mixture was concentrated to give a residue. The residue was triturated in CH₃CN (10 mL×2), and then concentrated to give a residue. Compound 2 (75 mg, yield: 94.3%) as yellow solid was obtained. ¹H NMR (400 MHz, DMSO-d₆) δ 9.35 (br s, 2H), 8.17-8.06 (m, 2H), 7.87 (br s, 1H), 7.32-7.12 (m, 5H), 5.53-5.29 (m, 1H), 3.74 (s, 3H), 3.28-2.86 (m, 10H). MS (ESI) m/z (M+H)⁺ 385.2.

### Example 4—Compounds 6-7

### N-(4-amino-3,4-dioxo-1-phenylbutan-2-yl)-3-(benzo[d]thiazol-7-yl)-1-methyl-1H-pyrazole-4-carboxamide (7)

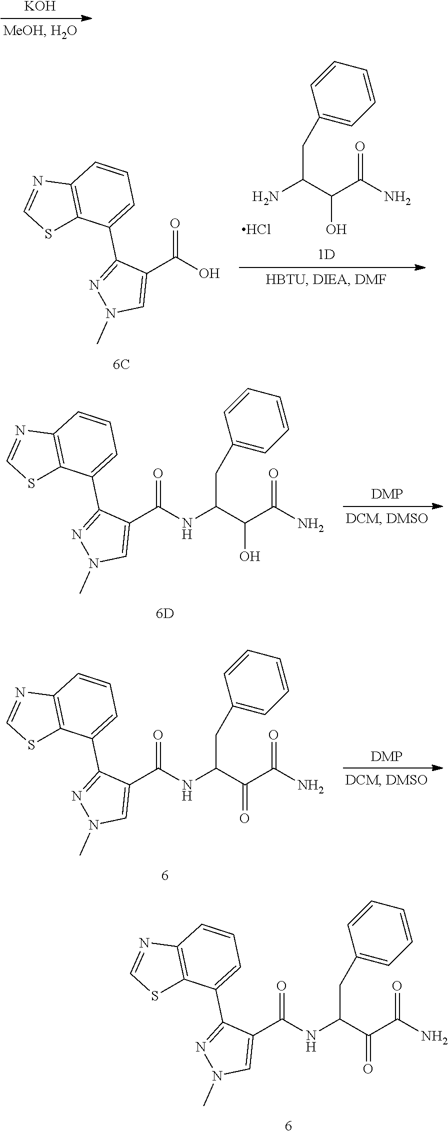

To a solution of 7-bromobenzo[d]thiazole (900 mg, 4.2 mmol) in dioxane (20 mL) was added KOAc (843 mg, 8.5 mmol), 4,4,4′,4′,5,5,5′,5′-octamethyl-2,2′-bi(1,3,2-dioxaborolane) (1.07 g, 4.2 mmol), Pd(dppf)Cl₂ (307 mg, 420 umol). Then the mixture was stirred at 90° C. for 12 h under N₂ atmosphere. The reaction was cooled to room temperature and the reaction was filtered. The filtered liquor was concentrated under reduced pressure to remove solvent. H₂O (20 mL) was added to the residue, the mixture was extracted with EA (20 mL×3). The combined organic layer was washed with brine (20 mL), dried over anhydrous Na₂SO₄, filtered and concentrated under reduced pressure to afford compound 6A (1.0 g, crude) as black oil which was used directly in next step.

Compound 6A was converted to compound 6 using procedures as described in Example 1. Compound 6 (50 mg, yield: 33%) as white solid was obtained. ¹H NMR (DMSO-d₆, 400 MHz): δ 9.36 (s, 1H), 8.60 (d, J=7.3 Hz, 1H), 8.14 (s, 1H), 8.10 (s, 1H), 8.03 (d, J=8.0 Hz, 1H), 7.83 (s, 1H), 7.78 (d, J=7.5 Hz, 1H), 7.48 (t, J=7.8 Hz, 1H), 7.33-7.27 (m, 4H), 7.26-7.20 (m, 1H), 5.41-5.22 (m, 1H), 3.97 (s, 3H), 3.18 (dd, J=3.8, 14.1 Hz, 1H), 2.83 (dd, J=10.2, 13.9 Hz, 1H). MS (ESI) m/z (M+H)⁺ 434.1.

### N-(4-amino-3,4-dioxo-1-phenylbutan-2-yl)-3-(benzo[d]thiazol-7-yl)-1-(difluoromethyl)-1H-pyrazole-4-carboxamide (7)

Compounds 6A and 4A were converted to compound 7 using procedures as described in Example 1. Compound 7 (60 mg, yield: 51.6%) as yellow solid was obtained. ¹H NMR (DMSO-d₆, 400 MHz): δ 9.41 (s, 1H), 8.99 (d, J=7.5 Hz, 1H), 8.59 (s, 1H), 8.17-8.09 (m, 2H), 8.02-7.83 (m, 2H), 7.73 (d, J=7.5 Hz, 1H), 7.53 (t, J=8.0 Hz, 1H), 7.30 (s, 4H), 7.24 (br s, 1H), 5.42-5.32 (m, 1H), 3.21 (br dd, J=3.3, 13.9 Hz, 1H), 2.82 (dd, J=10.1, 13.5 Hz, 1H). MS (ESI) m/z (M+H)⁺ 470.1.

### Example 5—Compounds 32, 62, 69, and 61

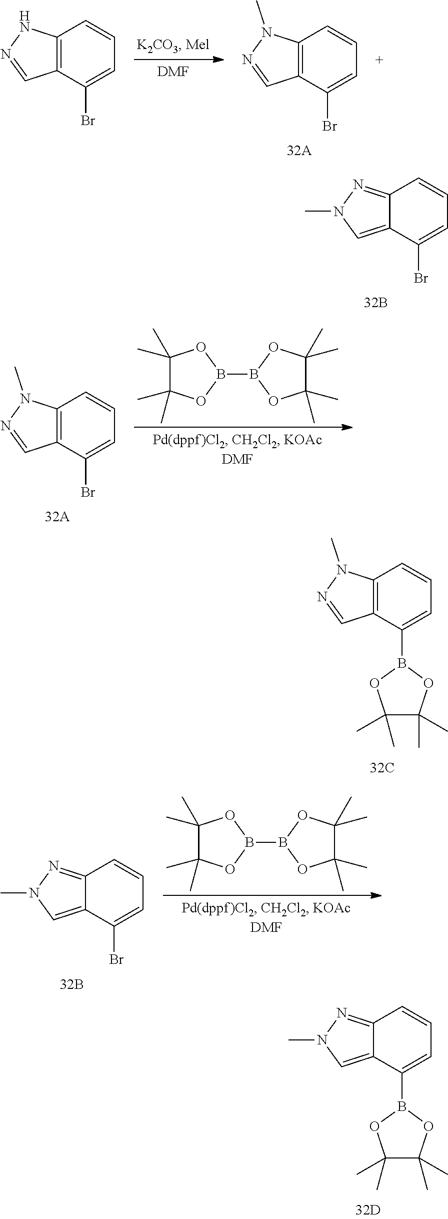

K₂CO₃ (5.26 g, 38.06 mmol) was added to a mixture of 4-bromo-1H-indazole (5 g, 25.38 mmol) in DMF (50 mL). 30 min later, MeI (18.2 g, 128.22 mmol, 8.0 mL) was added and the mixture was stirred at 25° C. for 3 h. The mixture was treated with H₂O (150 mL) and EA (50 mL). The organic layer was separated and the aqueous layer was extracted with EA (50 mL×2). The combined organic layer was washed brine (50 mL×2), dried over MgSO₄, filtered and concentrated. The residue was purified by flash column chromatography over silica gel (PE/EA=10/1 to 5/1) to afford a pair of isomers.

Isomer 1 (Compound 32A, R_{f}=0.54, PE/EA=5/1): 4-bromo-1-methyl-indazole (3.2 g, 59.8% yield) was obtained as white solid. ¹H NMR (DMSO-d₆, 400 MHz): δ 7.98 (d, J=0.9 Hz, 1H), 7.67-7.65 (m, 1H), 7.35-7.27 (m, 2H), 4.04 (s, 3H).

Isomer 2 (Compound 32B, R_{f}=0.24, PE/EA=5/1): 4-bromo-2-methyl-indazole (1.3 g, 24.3% yield) was obtained as colorless sticky oil. ¹H NMR (DMSO-d₆, 400 MHz): δ 8.37 (s, 1H), 7.60-7.57 (m, 1H), 7.26-7.21 (m, 1H), 7.13 (dd, J=7.3, 8.6 Hz, 1H), 4.16 (s, 3H).

KOAc (1.12 g, 11.37 mmol) was added to a mixture of compound 32A (1.2 g, 5.69 mmol) and 4,4,4′,4′,5,5,5′,5′-octamethyl-2,2′-bi(1,3,2-dioxaborolane) (2.17 g, 8.53 mmol) in DMF (25 mL), followed by Pd(dppf)Cl₂.CH₂Cl₂ (232 mg, 284.09 umol). Then nitrogen gas was bubbled through the mixture. The mixture was heated to 85° C. and stirred for 12 h. The mixture was treated with EA (75 mL) and brine (100 mL). The mixture was filtered through Celite. The filtrate was transferred to separating funnel. The organic layer was separated, dried over MgSO₄, filtered and concentrated. The residue was purified by silica gel column chromatography (petroleum ether/ethyl acetate=10/1 to 5/1) to afford compound 32C (1.5 g, 87.9% yield) as colorless sticky oil. ¹H NMR (DMSO-d₆, 400 MHz): δ 8.15 (d, J=0.8 Hz, 1H), 7.79 (d, J=8.5 Hz, 1H), 7.54-7.50 (m, 1H), 7.41 (dd, J=6.8, 8.5 Hz, 1H), 4.06 (s, 3H), 1.35 (s, 12H).

KOAc (1.2 g, 12.3 mmol) was added to mixture of compound 32B (1.3 g, 6.2 mmol) and 4,4,4′,4′,5,5,5′,5′-octamethyl-2,2′-bi(1,3,2-dioxaborolane) (2.4 g, 9.3 mmol) in DMF (20 mL). N₂ gas was bubbled through the mixture. Then Pd(dppf)Cl₂ CH₂Cl₂ (253 mg, 309.8 umol) was added. The mixture was stirred at 85° C. for 12 h under nitrogen atmosphere. The mixture was diluted with EA (50 mL) and brine (50 mL). The mixture was filtered through Celite. The filtrate was transferred to separating funnel. The organic layer was separated and the aqueous layer was extracted with EA (15 mL×2). The combined organic layer was washed with brine (35 mL), dried over MgSO₄, filtered and concentrated. The residue was purified by flash column chromatography over silica gel (PE/EA=5/1 to 2/1) to afford compound 32D (1.5 g, yield 94.4%) as white solid. MS (ESI) m/z (M+H)⁺ 259.2.

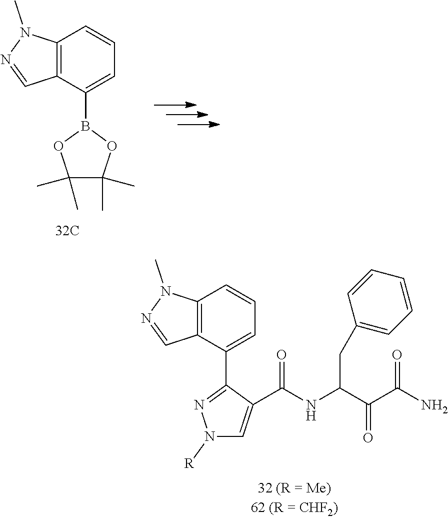

### N-(4-amino-3,4-dioxo-1-phenylbutan-2-yl)-1-methyl-3-(1-methyl-1H-indazol-4-yl)-1H-pyrazole-4-carboxamide (32)

Compounds 32C and ethyl 3-iodo-1-methyl-1H-pyrazole-4-carboxylate were converted to compound 32 using procedures as described in Example 1. Compound 32 (60 mg, yield: 60.0%) as pale yellow solid was obtained. ¹H NMR (DMSO-d₆, 400 MHz): δ 8.38 (br d, J=7.3 Hz, 1H), 8.09 (br d, J=9.5 Hz, 3H), 7.82 (br s, 1H), 7.61-7.53 (m, 1H), 7.35-7.19 (m, 7H), 5.38-5.25 (m, 1H), 4.05 (s, 3H), 3.96 (s, 3H), 3.15 (br dd, J=3.4, 13.7 Hz, 1H), 2.81 (br dd, J=10.2, 13.4 Hz, 1H). MS (ESI) m/z (M+H)⁺ 431.1.

### N-(4-amino-3,4-dioxo-1-phenylbutan-2-yl)-1-(difluoromethyl)-3-(1-methyl-1H-indazol-4-yl)-1H-pyrazole-4-carboxamide (62)

Compounds 32C and intermediate 4A were converted to compound 62 using procedures as described in Example 1. Compound 62 (96 mg, yield: 48.9%) as white solid was obtained. ¹H NMR (DMSO-d₆, 400 MHz): δ8.52 (s, 1H), 8.46 (d, J=9.8 Hz, 1H), 8.18-7.70 (m, 3H), 7.69-7.51 (m, 2H), 7.42-7.33 (m, 2H), 7.31-7.19 (m, 5H), 5.45-5.28 (m, 1H), 4.11-4.04 (m, 3H), 3.21 (dd, J=4.4, 14.2 Hz, 1H), 2.89 (dd, J=9.4, 14.2 Hz, 1H). MS (ESI) m/z (M+H)⁺ 467.1.

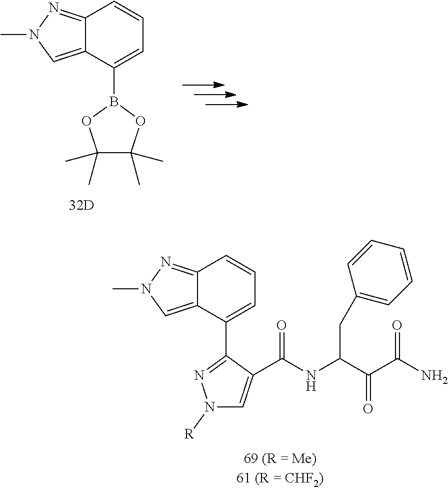

### N-(4-amino-3,4-dioxo-1-phenylbutan-2-yl)-1-methyl-3-(2-methyl-2H-indazol-4-yl)-1H-pyrazole-4-carboxamide (69)

Compounds 32D and ethyl 3-iodo-1-methyl-1H-pyrazole-4-carboxylate were converted to compound 69 using procedures as described in Example 1. Compound 69 (230 mg, yield: 69.7%) as white solid was obtained. ¹H NMR (400 MHz, DMSO-d₆) δ 8.39 (d, J=7.3 Hz, 1H), 8.36 (s, 1H), 8.10 (s, 1H), 8.06 (s, 1H), 7.85 (s, 1H), 7.53 (d, J=8.8 Hz, 1H), 7.32-7.22 (m, 6H), 7.15 (dd, J=7.2, 8.4 Hz, 1H), 5.33-5.28 (m, 1H), 4.17 (s, 3H), 3.95 (s, 3H), 3.16 (dd, J=3.9, 13.9 Hz, 1H), 2.81 (dd, J=9.9, 13.9 Hz, 1H). MS (ESI) m/z (M+H)⁺ 431.1.

### N-(4-amino-3,4-dioxo-1-phenylbutan-2-yl)-1-(difluoromethyl)-3-(2-methyl-2H-indazol-4-yl)-1H-pyrazole-4-carboxamide (61)

Compounds 32D and intermediate 4A were converted to compound 61 using procedures as described in Example 1. Compound 61 (250 mg, yield: 85.9%) as pale yellow solid was obtained. ¹H NMR (400 MHz, DMSO-d₆) δ 8.91 (d, J=7.5 Hz, 1H), 8.50 (s, 1H), 8.38 (s, 1H), 8.17-8.11 (m, 1H), 7.98-7.82 (m, 2H), 7.62 (d, J=8.5 Hz, 1H), 7.34-7.22 (m, 6H), 7.19 (dd, J=7.2, 8.4 Hz, 1H), 5.40-5.32 (m, 1H), 4.21-4.09 (m, 3H), 3.25-3.17 (m, 1H), 2.88-2.78 (m, 1H). MS (ESI) m/z (M+H)+=467.2.

### Example 6—Compounds 33-34, 77

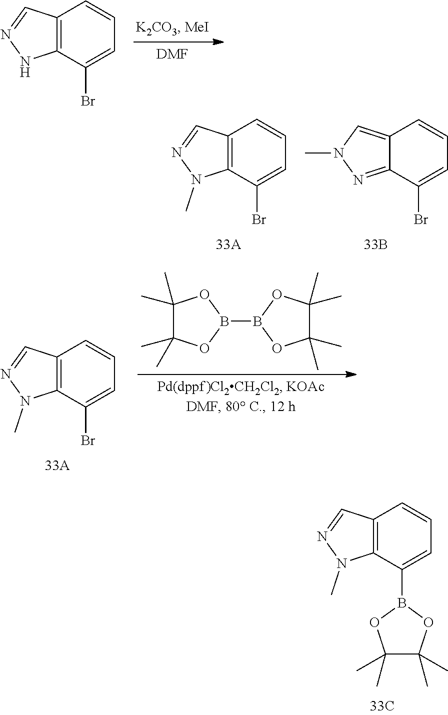

K₂CO₃ (3.51 g, 25.38 mmol) was added to a mixture of 7-bromo-1H-indazole (5 g, 25.38 mmol) in DMF (50 mL). 30 min later, MeI (18.05 g, 7.92 mL, 127.17 mmol,) was added and the mixture was stirred at 25° C. for 3 h. The insoluble substance was removed by filter. The filtrate was concentrated in vacuum. The residue was treated with H₂O (50 mL) and EA (50 mL). The organic layer was separated, washed with brine (15 mL×2), dried over MgSO₄, filtered and concentrated. The residue was purified by silica gel chromatography (PE/EA=10/1 to 3/1) to afford a pair of isomers.

Isomer 1 (Compound 33A, R_{f}=0.54, PE/EA=5/1): 7-bromo-1-methyl-1H-indazole (2.85 g, 53.2% yield) was obtained as colorless oil, which turned white solid after standing by. ¹H NMR (DMSO-d₆, 400 MHz): δ 8.09 (s, 1H), 7.74 (dd, J=0.9, 7.9 Hz, 1H), 7.56 (dd, J=0.8, 7.4 Hz, 1H), 7.02-6.97 (m, 1H), 4.28 (s, 3H).

Isomer 2 (Compound 33B, R_{f}=0.18, PE/EA=5/1): 7-bromo-2-methyl-2H-indazole (1.85 g, 34.5% yield) was obtained as white solid. ¹H NMR (DMSO-d₆, 400 MHz): δ 8.47 (s, 1H), 7.69 (dd, J=0.7, 8.4 Hz, 1H), 7.49-7.44 (m, 1H), 6.91 (dd, J=7.3, 8.2 Hz, 1H), 4.17 (s, 3H).

KOAc (1.35 g, 13.74 mmol) was added to a mixture of compound 33A (1.45 g, 6.87 mmol) and 4,4,4′,4′,5,5,5′,5′-octamethyl-2,2′-bi(1,3,2-dioxaborolane) (2.62 g, 10.31 mmol) in DMF (25 mL). Nitrogen gas was bubbled through the mixture and Pd(dppf)Cl₂.CH₂Cl₂ (280 mg, 342.87 umol) was added. Then the mixture was heated to 85° C. and stirred for 12 h. The mixture was treated with EA (75 mL) and brine (100 mL). The mixture was filtered through Celite. The filtrate was transferred separating funnel. The organic layer was separated, dried over MgSO₄, filtered and concentrated. The residue was purified by silica gel column chromatography (petroleum ether/ethyl acetate=10/1 to 5/1) to afford compound 33C (1.7 g, 90.1% yield) as white solid. ¹H NMR (DMSO-d₆, 400 MHz): δ 7.99 (s, 1H), 7.89 (dd, J=1.0, 7.0 Hz, 1H), 7.82 (dd, J=1.3, 8.0 Hz, 1H), 7.13 (dd, J=7.0, 8.0 Hz, 1H), 4.31 (s, 3H), 1.41 (s, 12H). MS (ESI) m/z (M+H)⁺ 259.2.

### N-(4-amino-3,4-dioxo-1-phenylbutan-2-yl)-1-methyl-3-(1-methyl-1H-indazol-7-yl)-1H-pyrazole-4-carboxamide (33)

Compounds 33C and ethyl 3-iodo-1-methyl-1H-pyrazole-4-carboxylate were converted to compound 33 using procedures as described in Example 1. Compound 33 (70 mg, yield: 43.6%) as pale yellow solid was obtained. ¹H NMR (DMSO-d₆, 400 MHz): δ 8.37 (s, 1H), 8.06 (s, 1H), 8.02 (s, 1H), 7.93 (d, J=7.8 Hz, 1H), 7.82-7.70 (m, 2H), 7.26-7.17 (m, 3H), 7.13-7.06 (m, 4H), 5.26-5.17 (m, 1H), 3.95 (s, 3H), 3.46 (s, 3H), 3.10 (br dd, J=3.4, 13.9 Hz, 1H), 2.69 (br dd, J=9.8, 13.8 Hz, 1H). MS (ESI) m/z (M+H)⁺ 431.2.

### N-(4-amino-3,4-dioxo-1-phenylbutan-2-yl)-1-(difluoromethyl)-3-(1-methyl-1H-indazol-7-yl)-1H-pyrazole-4-carboxamide (34)

Compounds 33C and intermediate 4A were converted to compound 34 using procedures as described in Example 1. Compound 34 (30 mg, yield: 27.0%) as a white solid was obtained. ¹H NMR (400 MHz, DMSO-d₆) δ 8.81 (s, 1H), 8.10-8.00 (m, 2H), 7.92-7.43 (m, 4H), 7.22-7.07 (m, 7H), 5.30-5.22 (m, 1H), 3.52 (s, 3H), 3.15 (d, J=10.0 Hz, 1H), 2.79 (dd, J=9.4, 13.9 Hz, 1H). MS (ESI) m/z (M+H)⁺ 467.2), 4.21-4.09 (m, 3H), 3.25-3.17 (m, 1H), 2.88-2.78 (m, 1H). MS (ESI) m/z (M+H)⁺=467.2.

### N-(4-amino-3,4-dioxo-1-phenylbutan-2-yl)-1-(difluoromethyl)-3-(2-methyl-2H-indazol-7-yl)-1H-pyrazole-4-carboxamide (77)

Compounds 2-methyl-7-(4,4,5,5-tetramethyl-1,3,2-dioxaborolan-2-yl)-2H-indazole (prepared from intermediate 33B using same procedure as 33C) and intermediate 4A were converted to compound 77 using procedures as described in Example 1. Compound 77 (30 mg, yield: 42.6%) as a white solid was obtained. ¹H NMR (400 MHz, DMSO-d₆) δ 8.59 (s, 1H), 8.40-8.35 (m, 2H), 8.05-7.88 (m, 2H), 7.77-7.73 (m, 2H), 7.22-7.11 (m, 4H), 7.08-7.02 (m, 1H), 7.00-6.95 (m, 2H), 5.25-5.18 (m, 1H), 4.03 (s, 3H), 3.06-2.99 (m, 1H), 2.61-2.53 (m, 1H). MS (ESI) m/z (M+H)⁺ 467.2.

### Example 7—Compounds 17, 31, 51, 70, 24, 26, and 55

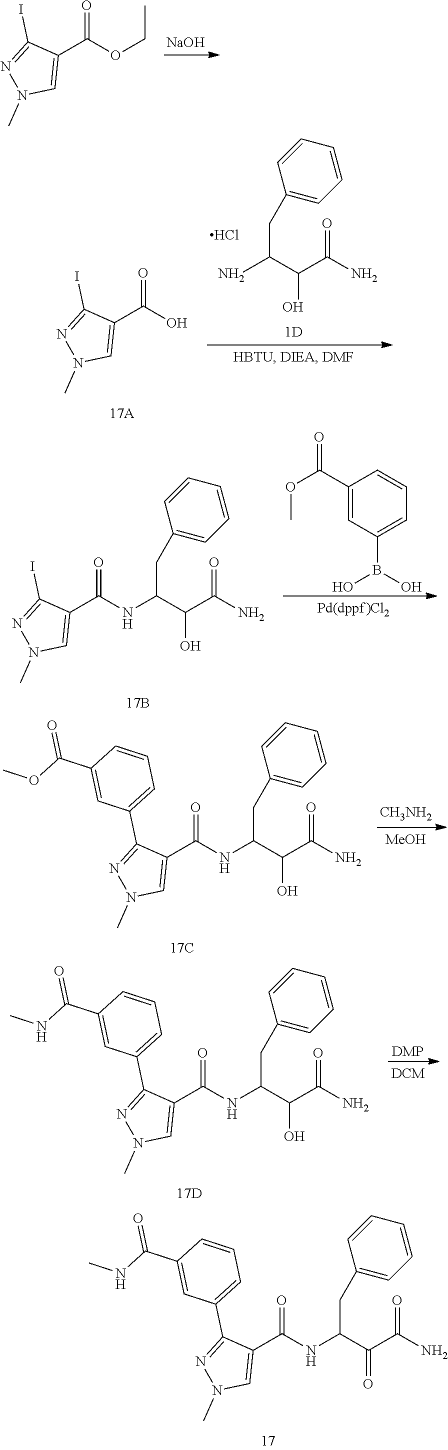

To a solution of ethyl 3-iodo-1-methyl-1H-pyrazole-4-carboxylate (1 g, 3.57 mmol) in MeOH (15 mL) was added the solution of NaOH (714 mg, 17.85 mmol) in H₂O (2 mL), the mixture was stirred at 50° C. for 1 h. The reaction mixture was concentrated to remove MeOH, then diluted with water (30 mL), acidified with 1N HCl to pH ˜3, the precipitate was formed, the solid was filtered and dried in vacuum. The residue was used into the next step without further purification. Compound 17A (850 mg, yield: 94.5%) as white solid was obtained. ¹H NMR (400 MHz, DMSO-d₆) δ 12.45 (s, 1H), 8.31-8.08 (m, 1H), 3.96-3.76 (m, 3H).

To a solution of compound 17A (0.85 g, 3.37 mmol) and Intermediate 1D (856 mg, 3.71 mmol, HCl) in DMF (20 mL) was added HBTU (1.53 g, 4.05 mmol) and DIEA (13.49 mmol, 2.35 mL), the mixture was stirred at 25° C. for 1 h. The reaction mixture was diluted with water (50 mL) at 0° C., the precipitate was formed, and the solid was filtered and dried in vacuum. The residue was used into the next step without further purification. Compound 17B (1.2 g, yield: 83.0%) as white solid was obtained. ¹H NMR (400 MHz, DMSO-d₆) δ 8.13 (s, 1H), 7.62 (d, J=9.0 Hz, 1H), 7.33 (s, 2H), 7.29-7.17 (m, 4H), 7.16-7.09 (m, 1H), 5.87 (d, J=6.0 Hz, 1H), 4.56-4.36 (m, 1H), 4.01 (dd, J=3.3, 5.7 Hz, 1H), 3.84 (s, 3H), 2.89-2.62 (m, 2H). MS (ESI) m/z (M+H)⁺ 429.0.

To a solution of compound 17B (1.2 g, 2.80 mmol) and (3-methoxycarbonylphenyl)boronic acid (756 mg, 4.20 mmol) in dioxane (30 mL) and H₂O (3 mL) was added K₂CO₃ (775 mg, 5.60 mmol), then Pd(dppf)Cl₂ (205 mg, 280.23 umol) was added under N₂ atmosphere, the mixture was stirred at 80° C. for 18 h. The reaction mixture was concentrated to remove solvent, diluted with EA (50 mL), filtered and washed with EA (20 mL×2), the filtrate was washed with water (50 mL×2), then dried over Na₂SO₄, filtered and concentrated to give a residue. The residue was purified by flash silica gel chromatography (ISCO®; 12 g SepaFlash® Silica Flash Column, Eluent of 0-100% Ethyl acetate/Petroleum ether gradient to EA:MeOH=10:1 @ 30 mL/min). Compound 17C (0.4 g, yield: 32.7%) as yellow solid was obtained. ¹H NMR (400 MHz, DMSO-d₆) δ 8.29 (t, J=1.7 Hz, 1H), 8.07 (s, 1H), 7.90-7.80 (m, 2H), 7.77-7.74 (m, 1H), 7.46-7.39 (m, 1H), 7.34-7.11 (m, 7H), 5.82 (d, J=5.7 Hz, 1H), 4.58-4.40 (m, 1H), 4.02 (dd, J=3.5, 5.7 Hz, 1H), 3.89 (s, 3H), 3.85 (s, 3H), 2.87-2.66 (m, 2H).

To a solution of compound 17C (120 mg, 274.94 umol) in MeOH (3 mL) was added CH₃NH₂ (549.88 umol, 8 mL), then the mixture was stirred at 45° C. for 40 h. The reaction mixture was concentrated to remove solvent, diluted with DCM (20 mL) and filtered, the solid was collected. The residue was purified by preparatory-HPLC (column: YMC-Actus Triart C18 100*30 mm*5 um; mobile phase: [water (0.05% HCl)-ACN]; B %: 10%-66%, 8.5 min). Compound 17D (60 mg, yield 49.8%) as white solid was obtained. ¹H NMR (400 MHz, DMSO-d₆) δ 8.43 (br d, J=4.6 Hz, 1H), 8.08 (d, J=18.1 Hz, 2H), 7.75 (dd, J=8.6, 11.0 Hz, 2H), 7.58 (d, J=7.7 Hz, 1H), 7.41-7.11 (m, 8H), 4.47 (br s, 1H), 4.02 (d, J=3.7 Hz, 1H), 3.89 (s, 3H), 2.82-2.65 (m, 5H). MS (ESI) m/z (M+H)⁺ 436.1.

To a solution of compound 17D (60 mg, 137.78 umol) in DMSO (3 mL) and DCM (50 mL) was added DMP (234 mg, 551.12 umol), the mixture was stirred at 25° C. for 1 h. The reaction mixture was diluted with DCM (20 mL) and quenched by addition Na₂S₂O₃ (sat, 30 mL) and NaHCO₃ (saturated 30 mL), the mixture was extracted with DCM (30 mL×2). The combined organic layers were washed with H₂O (50 mL), then washed with brine (50 mL×2), dried over Na₂SO₄, filtered and concentrated under reduced pressure to give a residue. The residue was triturated in CH₃CN, filtered and the solid was dried in vacuum. Compound 17 (15 mg, yield: 22.8%) as white solid was obtained. ¹H NMR (400 MHz, DMSO-d₆) δ 8.48-8.35 (m, 2H), 8.15-8.07 (m, 2H), 8.04 (s, 1H), 7.80 (s, 1H), 7.74 (td, J=1.5, 7.8 Hz, 1H), 7.64 (td, J=1.4, 8.0 Hz, 1H), 7.36 (t, J=7.8 Hz, 1H), 7.32-7.17 (m, 5H), 5.30-5.24 (m, 1H), 3.91 (s, 3H), 3.15 (dd, J=4.0, 13.9 Hz, 1H), 2.89-2.74 (m, 4H). MS (ESI) m/z (M+H)⁺ 434.2.

### N-(4-amino-3,4-dioxo-1-phenylbutan-2-yl)-3-(benzo[d]oxazol-7-yl)-1-methyl-1H-pyrazole-4-carboxamide (31)

Compounds 7-(4,4,5,5-tetramethyl-1,3,2-dioxaborolan-2-yl)benzo[d]oxazole (prepared from 7-bromobenzo[d]oxazole using same procedure as 33C) and intermediate 17B were converted to compound 31 using procedures as described in Example 1. Compound 31 (60 mg, yield: 60.2%) as a white solid was obtained. ¹H NMR (400 MHz, DMSO-d₆) δ 8.61 (s, 1H), 8.44 (d, J=7.6 Hz, 1H), 8.21 (s, 1H), 8.03 (s, 1H), 7.80-7.74 (m, 2H), 7.47-7.43 (m, 1H), 7.39-7.20 (m, 6H), 5.26-5.19 (m, 1H), 3.96 (s, 3H), 3.17-3.10 (m, 1H), 2.86-2.79 (m, 1H). MS (ESI) m/z (M+H)⁺ 418.1.

### N-(4-amino-3,4-dioxo-1-phenylbutan-2-yl)-3-(benzo[d]thiazol-4-yl)-1-methyl-1H-pyrazole-4-carboxamide (51)

Compounds benzo[d]thiazol-4-ylboronic acid (prepared from 4-bromobenzo[d]thiazole using same procedure as 33C) and intermediate 17B were converted to compound 51 using procedures as described in Example 1. Compound 51 (75 mg, yield: 69.6%) as a pale yellow solid was obtained. ¹H NMR (DMSO-d₆, 400 MHz): δ 9.19 (s, 1H), 8.22-8.12 (m, 2H), 7.99-7.90 (m, 2H), 7.73 (s, 1H), 7.51-7.42 (m, 2H), 7.27-7.15 (m, 3H), 7.13-7.06 (m, 2H), 5.22-5.06 (m, 1H), 3.92 (s, 3H), 3.11-2.94 (m, 1H), 2.80-2.63 (m, 1H). MS (ESI) m/z (M+H)⁺ 434.1.

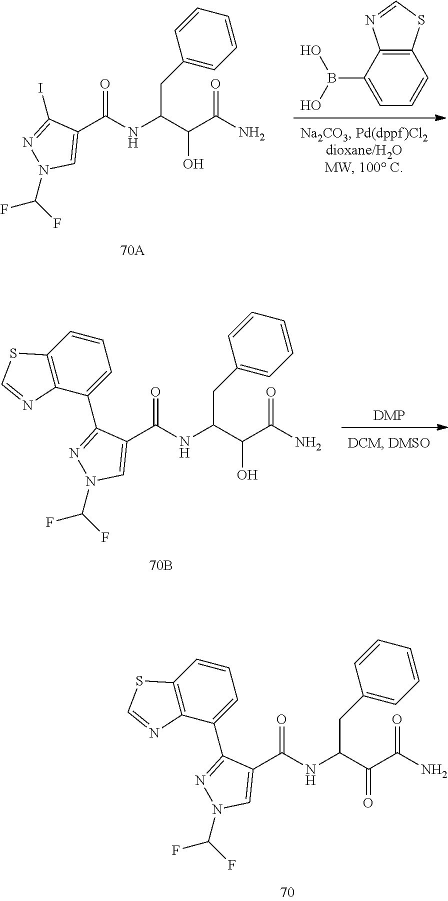

### N-(4-amino-3,4-dioxo-1-phenylbutan-2-yl)-3-(benzo[d]thiazol-4-yl)-1-(difluoromethyl)-1H-pyrazole-4-carboxamide (70)

Compounds benzo[d]thiazol-4-ylboronic acid (prepared from 4-bromobenzo[d]thiazole using same procedure as 33C) and intermediate 70A (prepared from 4A using same procedure as 17B) were converted to compound 70 using procedures as described in Example 1. Compound 70 (50 mg, yield: 48.5%) as a pale yellow solid was obtained. ¹H NMR (DMSO-d₆, 400 MHz): δ 9.14 (s, 1H), 8.61 (s, 1H), 8.52 (d, J=7.3 Hz, 1H), 8.20-8.16 (m, 1H), 8.11-7.87 (m, 2H), 7.79-7.69 (m, 1H), 7.50-7.44 (m, 2H), 7.27-7.13 (m, 5H), 5.16-5.07 (m, 1H), 3.04 (dd, J=3.7, 13.9 Hz, 1H), 2.72 (dd, J=9.7, 13.9 Hz, 1H). MS (ESI) m/z (M+H)⁺ 470.1.

### N-(4-amino-3,4-dioxo-1-phenylbutan-2-yl)-1-(difluoromethyl)-3-(2,5-dimethylfuran-3-yl)-1H-pyrazole-4-carboxamide (24)

Compounds 2-(2,5-dimethylfuran-3-yl)-4,4,5,5-tetramethyl-1,3,2-dioxaborolane and intermediate 70A (prepared from 4A using same procedure as 17B) were converted to compound 24 using procedures as described in Example 1. Compound 24 (140 mg, yield: 79.8%) as a light yellow solid was obtained. ¹H NMR (400 MHz, DMSO-d₆) δ 8.67-8.56 (m, 1H), 8.49 (s, 1H), 8.11 (s, 1H), 8.04-7.67 (m, 2H), 7.35-7.16 (m, 5H), 6.09 (s, 1H), 5.35-5.29 (m, 1H), 3.18 (dd, J=4.0, 14.1 Hz, 1H), 2.81 (dd, J=9.9, 13.9 Hz, 1H), 2.20 (d, J=12.1 Hz, 6H). MS (ESI) m/z (M+H)±431.1.

### N-(4-amino-3,4-dioxo-1-phenylbutan-2-yl)-1-(difluoromethyl)-3-(2-Methylfuran-3-yl)-1H-pyrazole-4-carboxamide (26)

Compounds 4,4,5,5-tetramethyl-2-(2-methylfuran-3-yl)-1,3,2-dioxaborolane and intermediate 70A (prepared from 4A using same procedure as 17B) were converted to compound 26 using procedures as described in Example 1. Compound 26 (128 mg, yield: 95.87%) as a pale yellow solid was obtained. ¹H NMR (400 MHz, DMSO-d₆) δ 8.63 (d, J=7.3 Hz, 1H), 8.50 (s, 1H), 8.11-7.67 (m, 3H), 7.44 (d, J=1.8 Hz, 1H), 7.30-7.22 (m, 4H), 7.22-7.15 (m, 1H), 6.49 (d, J=1.8 Hz, 1H), 5.37-5.23 (m, 1H), 3.16 (dd, J=3.6, 14.0 Hz, 1H), 2.79 (br dd, J=10.1, 13.9 Hz, 1H), 2.25 (s, 3H). MS (ESI) m/z (M+H)⁺ 417.1.

### N-(4-amino-3,4-dioxo-1-phenylbutan-2-yl)-3-(2,5-dimethylfuran-3-yl)-1-methyl-1H-pyrazole-4-carboxamide (55)

Compounds 2-(2,5-dimethylfuran-3-yl)-4,4,5,5-tetramethyl-1,3,2-dioxaborolane and intermediate 17B were converted to compound 55 using procedures as described in Example 1. Compound 55 (22 mg, yield: 26.5%) as a white solid was obtained. ¹H NMR (400 MHz, DMSO-d₆) δ 8.09-8.03 (m, 2H), 8.01 (d, J=7.3 Hz, 1H), 7.81 (s, 1H), 7.32-7.25 (m, 2H), 7.25-7.17 (m, 3H), 6.13-6.02 (s, 1H), 5.28 (m, 1H), 3.84 (s, 3H), 3.15 (dd, J=4.0, 13.9 Hz, 1H), 2.82 (dd, J=9.7, 13.9 Hz, 1H), 2.23-2.12 (m, 6H). MS (ESI) m/z (M+H)⁺395.2.

### Example 8—Compounds 68 and 71

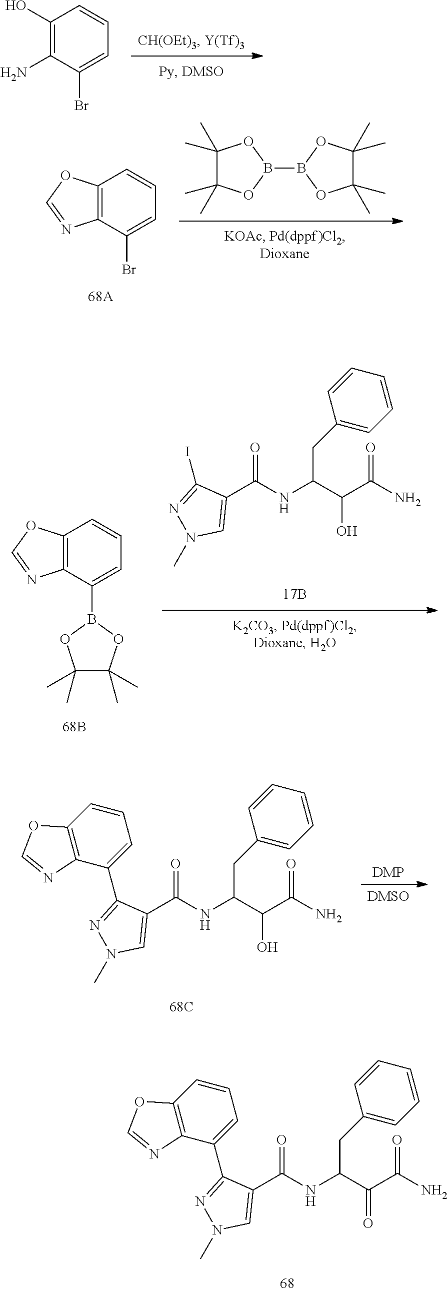

Yttrium tris (trifluoromethanesulfonate) (249 mg, 0.5 mmol) and Triethylorthoformate (15 mL, 93.1 mmol) were combined. To this mixture was added a solution of 2-amino-3-bromophenol (1.8 g, 9.31 mmol) in DMSO (20 mL) and Pyridine (1.5 mL, 18.6 mmol). The reaction mixture was stirred in a heat block at 60° C. for 18 h. The mixture was added H₂O (200 mL) and extracted with EA (50 mL). The organic phase was washed with brine (20 mL) and dried over Na₂SO₄, filtered and concentrated under vacuum. The product was purified by FCC (0-50% EA/PE) to afford compound 68A (1 g, yield 51.7%) as a red solid. ¹H NMR (400 MHz, DMSO-d₆) δ 8.96 (s, 1H), 7.90 (d, J=8.2 Hz, 1H), 7.73 (d, J=7.6 Hz, 1H), 7.53-7.44 (m, 1H). MS (ESI) m/z (M+H)⁺ 198.0.

### N-(4-amino-3,4-dioxo-1-phenylbutan-2-yl)-3-(benzo[d]oxazol-4-yl)-1-methyl-1H-pyrazole-4-carboxamide (68)

Compounds 4-(4,4,5,5-tetramethyl-1,3,2-dioxaborolan-2-yl)benzo[d]oxazole (68B) (prepared from 68A using same procedure as 33C) and intermediate 17B were converted to compound 68 using procedures as described in Example 1. Compound 68 (10 mg, yield: 6.7%) as a white solid was obtained. ¹H NMR (400 MHz, DMSO-d₆) δ 8.52 (s, 1H), 8.15 (s, 1H), 7.73 (dd, J=1.6, 7.7 Hz, 1H), 7.69-7.46 (m, 3H), 7.45-7.37 (m, 2H), 7.25-7.15 (m, 3H), 7.08 (d, J=6.3 Hz, 2H), 5.26-5.21 (m, 1H), 3.94 (s, 3H), 3.22-3.10 (m, 1H), 2.83 (dd, J=8.5, 14.1 Hz, 1H). MS (ESI) m/z (M+H)⁺ 418.1.

### N-(4-amino-3,4-dioxo-1-phenylbutan-2-yl)-3-(benzo[d]oxazol-4-yl)-1-(difluoromethyl)-1H-pyrazole-4-carboxamide (71)

Compounds 68B and intermediate 70A (prepared from 4A using same procedure as 17B) were converted to compound 71 using procedures as described in Example 1. Compound 71 (124 mg, yield: 77.99%) as a pale yellow solid was obtained. ¹H NMR (400 MHz, DMSO-d₆) δ 8.65 (s, 1H), 8.55 (s, 1H), 8.19 (s, 1H), 8.10-7.88 (m, 2H), 7.79 (dd, J=2.9, 6.4 Hz, 1H), 7.75-7.64 (m, 1H), 7.53-7.44 (m, 2H), 7.30-7.14 (m, 5H), 5.30-5.21 (m, 1H), 3.17-3.12 (m, 1H), 2.87 (dd, J=8.9, 14.2 Hz, 1H). MS (ESI) m/z (M+H)⁺ 454.1.

### Example 9—Compounds 35 and 50

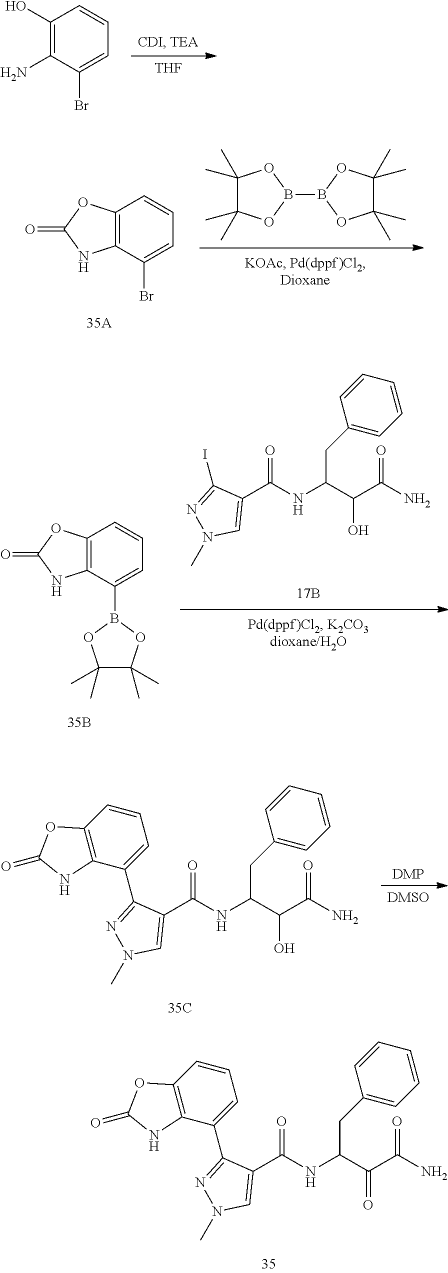

TEA (1.5 mL, 10.64 mmol) was added to the mixture of 2-amino-3-bromophenol (1 g, 5.32 mmol) and CDI (1.72 g, 10.64 mmol) in THF (20 mL). The mixture was stirred at 60° C. for 18 h. The reaction mixture was evaporated and diluted with dichloromethane (60 mL). The organic layer was washed with 1M hydrochloric acid (2×30 mL) and water (30 mL). The organic layer was dried over sodium sulfate, filtered and concentrated under vacuo. Compound 35A (1.1 g, 96.64% yield) was obtained as a red solid, which was used for next step directly. ¹H NMR (400 MHz, DMSO-d₆) δ 12.19 (br s, 1H), 7.37-7.29 (m, 2H), 7.08-7.01 (m, 1H).

### N-(4-amino-3,4-dioxo-1-phenylbutan-2-yl)-1-methyl-3-(2-oxo-2,3-dihydrobenzo[d]oxazol-4-yl)-1H-pyrazole-4-carboxamide (35)

Compounds 4-(4,4,5,5-tetramethyl-1,3,2-dioxaborolan-2-yl)benzo[d]oxazol-2(3H)-one (35B) (prepared from 35A using same procedure as 33C) and intermediate 17B were converted to compound 35 using procedures as described in Example 1. Compound 35 (18 mg, yield: 29.62%) as a yellow solid was obtained. ¹H NMR (400 MHz, DMSO-d₆) δ 9.47 (br s, 1H), 7.88 (s, 1H), 7.55 (d, J=8.3 Hz, 1H), 7.36-7.21 (m, 5H), 7.18 (d, J=8.0 Hz, 1H), 7.06 (br t, J=8.2 Hz, 2H), 6.96 (br d, J=6.8 Hz, 1H), 6.25 (br s, 1H), 5.49-5.40 (m, 1H), 4.01-3.93 (m, 3H), 3.30 (dd, J=4.8, 14.1 Hz, 1H), 2.93 (dd, J=9.0, 14.1 Hz, 1H). MS (ESI) m/z (M+H)⁺ 434.2.

### N-(4-amino-3,4-dioxo-1-phenylbutan-2-yl)-1-(difluoromethyl)-3-(2-oxo-2,3-dihydrobenzo[d]oxazol-4-yl)-1H-pyrazole-4-carboxamide (50)

Compounds 35B and intermediate 70A (prepared from 4A using same procedure as 17B) were converted to compound 50 using procedures as described in Example 1. Compound 50 (20 mg, yield: 22.8%) as a white solid was obtained. ¹H NMR (400 MHz, DMSO-d₆) δ 11.27 (s, 1H), 8.46 (s, 1H), 8.12-7.90 (m, 1H), 7.83-7.58 (m, 2H), 7.23-6.59 (m, 9H), 5.24 (s, 1H), 2.99-2.97 (m, 1H), 2.70-2.60 (m, 1H). MS (ESI) m/z (M+H)⁺ 470.1.

### Example 10—Compound 16

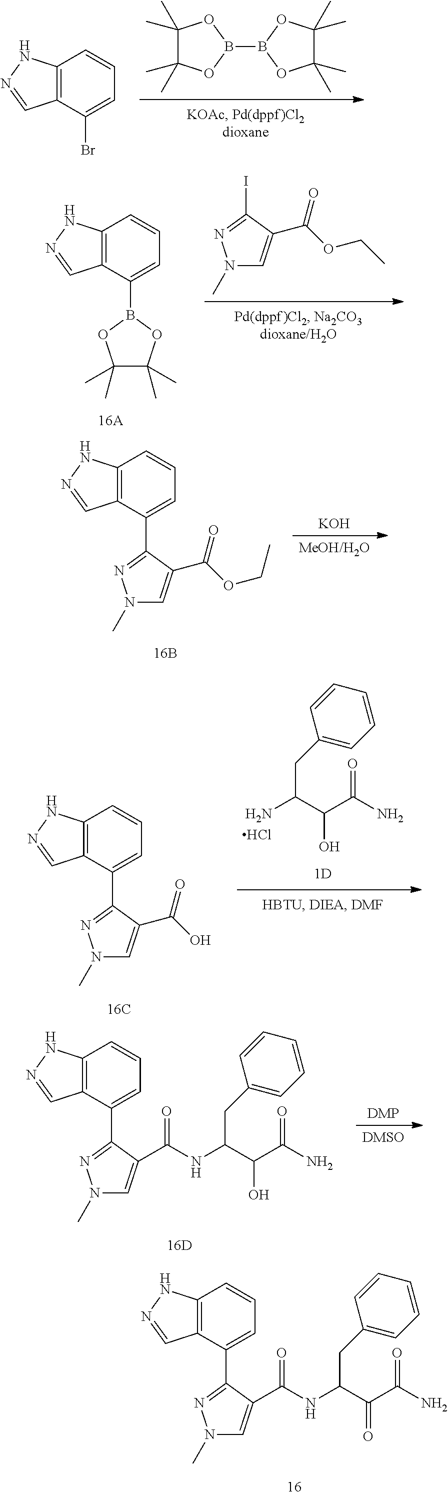

### N-(4-amino-3,4-dioxo-1-phenylbutan-2-yl)-3-(1H-indazol-4-yl)-1-methyl-1H-pyrazole-4-carboxamide (16)

Compounds 4-(4,4,5,5-tetramethyl-1,3,2-dioxaborolan-2-yl)-1H-indazole (16A) (prepared from 4-bromo-1H-indazole using same procedure as 33C) and ethyl 3-iodo-1-methyl-1H-pyrazole-4-carboxylate were converted to compound 16 using procedures as described in Example 1. Compound 16 (60 mg, yield: 77.4%) as a white solid was obtained. ¹H NMR (DMSO-d₆, 400 MHz): δ 13.05 (br s, 1H), 8.34 (d, J=7.3 Hz, 1H), 8.13-8.08 (m, 2H), 8.06 (s, 1H), 7.81 (s, 1H), 7.52-7.45 (m, 1H), 7.32-7.19 (m, 7H), 5.34-5.24 (m, 1H), 3.95 (s, 3H), 3.14 (dd, J=3.8, 14.1 Hz, 1H), 2.80 (dd, J=9.9, 13.9 Hz, 1H). MS (ESI) m/z (M+H)⁺ 417.1.

### Example 11—Compound 39

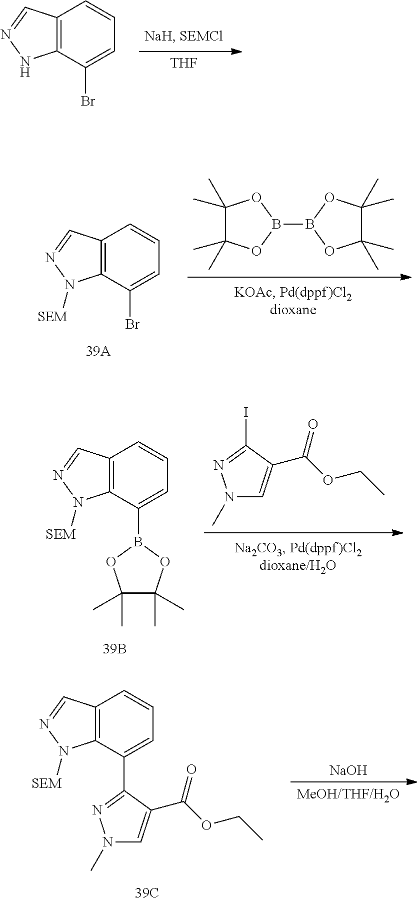

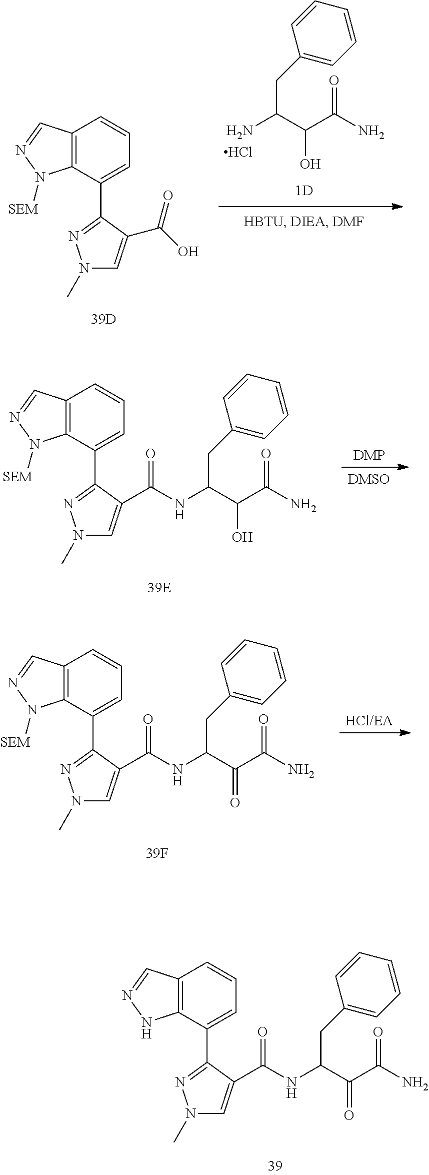

### N-(4-amino-3,4-dioxo-1-phenylbutan-2-yl)-3-(1H-indazol-7-yl)-1-methyl-1H-pyrazole-4-carboxamide (39)

NaH (406 mg, 10.2 mmol, 60% purity) was added to a mixture of 7-bromo-1H-indazole (1 g, 5.1 mmol) in THF (15 mL) at 0° C. The mixture was stirred at 0° C. for 1 h, then SEM-Cl (1.35 mL, 7.62 mmol) was added. After addition, the reaction temperature was allow to rise to room temperature (22° C.) slowly and the mixture was stirred for 15 h at 22° C. The mixture was quenched with the addition of saturated NH₄Cl (30 mL). Then the mixture was extracted with EA (3×25 mL). The combined organic layer was washed with brine (20 mL), dried over anhydrous MgSO₄, filtered and concentrated. The residue was purified by silica gel column chromatography (petroleum ether/ethyl acetate=1/0 to 8/1) to afford compound 39A (1.1 g, yield 66.2%) as yellow oil. ¹H NMR (400 MHz, DMSO-d₆) δ 8.26 (s, 1H), 7.85 (dd, J=0.9, 7.9 Hz, 1H), 7.70 (dd, J=0.9, 7.5 Hz, 1H), 7.13 (t, J=7.7 Hz, 1H), 5.99 (s, 2H), 3.52 (t, J=7.8 Hz, 2H), 0.78 (t, J=7.8 Hz, 2H), −0.13 (s, 9H).

Compounds 7-(4,4,5,5-tetramethyl-1,3,2-dioxaborolan-2-yl)-1-1-((2-(trimethylsilyl)ethoxy)methyl)-1H-indazole (39B) (prepared from 39A using same procedure as 33C) and ethyl 3-iodo-1-methyl-1H-pyrazole-4-carboxylate were converted to compound 39F using procedures as described in Example 1. Compound 39F (203 mg, yield: 70.49%) as a yellow solid was obtained. ¹H NMR (400 MHz, DMSO-d₆) δ 8.31 (s, 1H), 8.19-8.16 (m, 1H), 7.86-7.80 (m, 1H), 7.71-7.50 (m, 2H), 7.25-7.13 (m, 6H), 7.01 (d, J=7.3 Hz, 2H), 5.31 (s, 2H), 5.28-5.19 (m, 1H), 3.94 (s, 3H), 2.74 (dd, J=8.5, 14.1 Hz, 1H), 0.90-0.83 (m, 3H), 0.57 (t, J=8.0 Hz, 2H), −0.14 (s, 9H).

HCl/EtOAc (4M, 4 mL) was added to the mixture of Compound 39F (160 mg, 0.3 mmol). The mixture was stirred at 30° C. for 3 h. The mixture was filtered and the filtered cake was concentrated under vacuo. Compound 39 (66 mg, 54.1% yield) was obtained as a white solid. ¹H NMR (400 MHz, DMSO-d₆) δ 12.74 (s, 1H), 8.44 (d, J=7.5 Hz, 1H), 8.11-8.04 (m, 3H), 7.81-7.73 (m, 2H), 7.68 (d, J=7.5 Hz, 1H), 7.28-7.22 (m, 4H), 7.21-7.16 (m, 1H), 7.02 (t, J=7.6 Hz, 1H), 5.33-5.26 (m, 1H), 3.97 (s, 3H), 3.14 (dd, J=3.9, 14.0 Hz, 1H), 2.85-2.75 (m, 1H). MS (ESI) m/z (M+H)⁺=417.1.

### Example 12—Compounds 9, 47, and 48

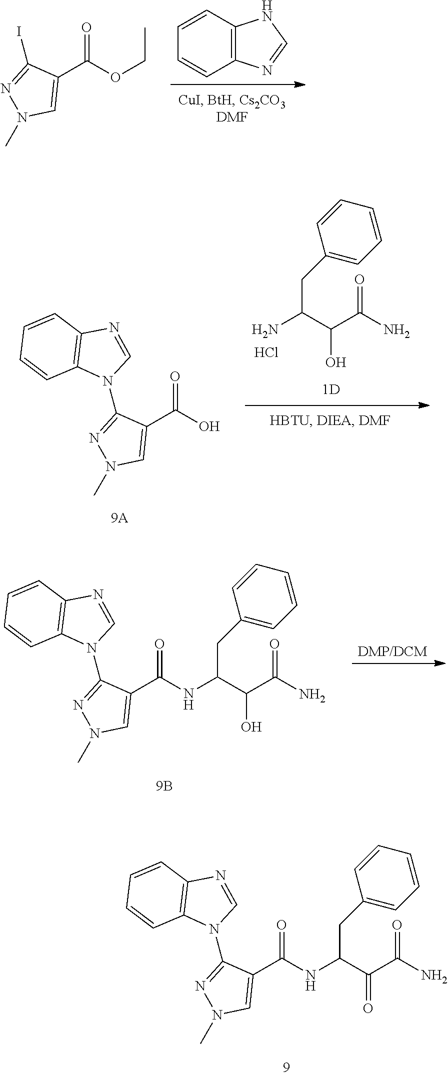

To a solution of ethyl 3-iodo-1-methyl-1H-pyrazole-4-carboxylate (4 g, 14.28 mmol) and 1H-benzo[d]imidazole (2 g, 16.93 mmol) in DMF (40 mL) was added Cs₂CO₃ (9.31 g, 28.57 mmol), 1H-benzotriazole (340 mg, 2.86 mmol) and CuI (272 mg, 1.43 mmol). The mixture was stirred at 110° C. for 48 h under N₂. The mixture was diluted with H₂O (100 mL), washed with EtOAc (150 mL). The aqueous phase was collected, adjusted to pH ˜4 with 1N HCl, washed with EtOAc (300 mL). The aqueous phase was collected and concentrated in vacuo. The residue was triturated with MeOH (40 mL). The solid was filtered off. The filtrate was collected and concentrated. The residue was purified by preparatory-HPLC (HCl) to give compound 9A (380 mg, yield: 10.74%) as white solid. MS (ESI) m/z (M+H)⁺ 242.9.

### N-(4-amino-3,4-dioxo-1-phenylbutan-2-yl)-3-(1H-benzo[d]imidizol-1-yl)-1-methyl-1H-pyrazole-4-carboxamide (9)

Compounds 49A and intermediate 1D were converted to compound 9 using procedures as described in Example 1. Compound 9 (70 mg, yield: 46.85%) as a white solid was obtained. MS (ESI) m/z (M+H)⁺417.1. ¹HNMR (400 MHz, DMSO-d₆) δ 8.61 (d, J=7.6 Hz, 1H), 8.39 (s, 1H), 8.32 (s, 1H), 8.02 (br. s, 1H), 7.77 (br. s, 1H), 7.71-7.65 (m, 1H), 7.50-7.43 (m, 1H), 7.30-7.16 (m, 7H), 5.29-5.20 (m, 1H), 4.00-3.91 (m, 3H), 3.18-3.09 (m, 1H), 2.85-2.75 (m, 1H).

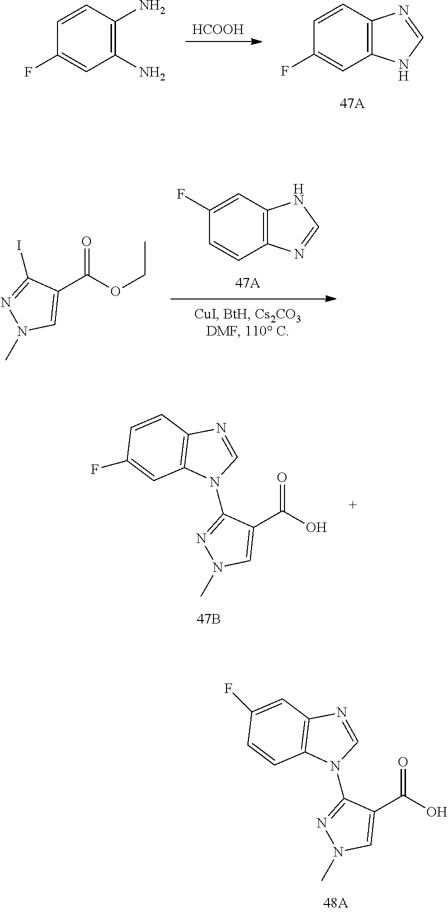

A mixture of 4-fluorobenzene-1,2-diamine (1 g, 7.93 mmol) and HCOOH (10 mL) was stirred at 90° C. for 2 h. The solution was adjusted to pH ˜7 with 5N NaOH. The mixture was extracted with EtOAc (50 mL×3). The organics were collected, dried with Na₂SO₄, filtered and concentrated to give compound 47A (1 g, crude) as brown solid, which was used directly for the next step without further purification.

Ethyl 3-iodo-1-methyl-1H-pyrazole-4-carboxylate and intermediate 47A were subjected to reaction conditions as for intermediate 9A and the reaction yielded products 47B and 48A. The product was purified by preparatory-HPLC (HCl) to give 400 mg of mixture as brown solid, which was repurified by SFC (column: AD (250 mm*30 mm, 5 um); mobile phase: [0.1% NH₃H₂O MEOH]; B %: 25%-25%, min) to give compound 47B (100 mg, yield: 2.61%) as white solid; compound 48A (100 mg, yield: 2.61%) as white solid, which was repurified by SFC to give 48A (90 mg). MS (ESI) m/z (M+H)⁺ 260.9.

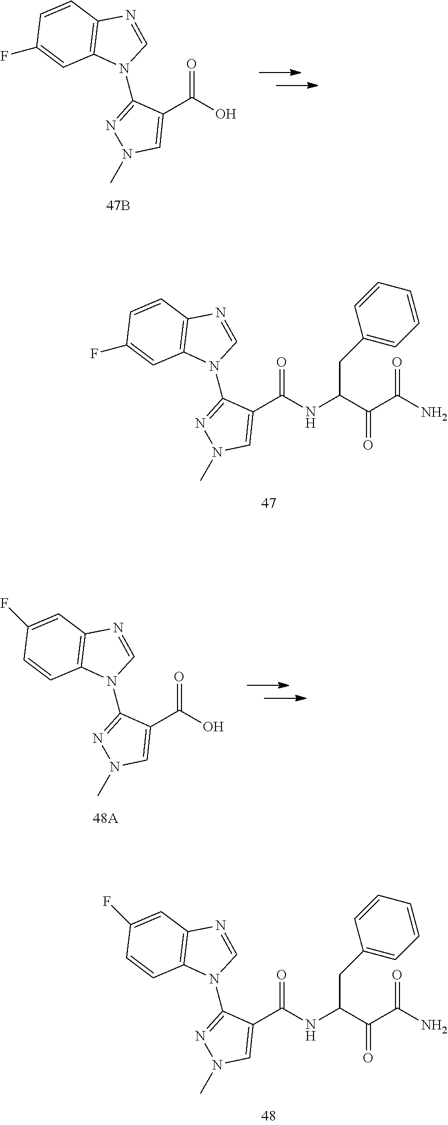 [SMILES: Cn1cc(C(=O)O)c(-n2cnc3ccc(F)cc32)n1 ; Cn1cc(C(=O)NC(Cc2ccccc2)C(=O)C(N)=O)c(-n2cnc3ccc(F)cc32)n1 ; Cn1cc(C(=O)O)c(-n2cnc3cc(F)ccc32)n1 ; Cn1cc(C(=O)NC(Cc2ccccc2)C(=O)C(N)=O)c(-n2cnc3cc(F)ccc32)n1]

### N-(4-amino-3,4-dioxo-1-phenylbutan-2-yl)-3-(6-fluoro-1H-benzo[d]imidizol-1-yl)-1-methyl-1H-pyrazole-4-carboxamide (47)

Compounds 47B and intermediate 1D were converted to compound 47 using procedures as described in Example 1. Compound 47 (50 mg, yield: 48.0%) as a white solid was obtained. ¹H NMR (400 MHz, DMSO-d₆) δ 8.42 (s, 1H), 8.36 (s, 1H), 8.33-8.27 (m, 1H), 7.72 (br s, 1H), 7.58-7.44 (m, 3H), 7.32-7.17 (m, 5H), 7.16-7.07 (m, 1H), 5.34-5.26 (m, 1H), 3.97 (s, 3H), 3.24-3.17 (m, 1H), 2.95-2.85 (m, 1H). MS (ESI) m/z (M+H)⁺ 435.2.

### N-(4-amino-3,4-dioxo-1-phenylbutan-2-yl)-3-(5-fluoro-1H-benzo[d]imidizol-1-yl)-1-methyl-1H-pyrazole-4-carboxamide (48)

Compounds 48A and intermediate 1D were converted to compound 48 using procedures as described in Example 1. Compound 48 (40 mg, yield: 28.2%) as a white solid was obtained. ¹H NMR (400 MHz, DMSO-d₆) δ 8.46-8.21 (m, 3H), 7.80-7.41 (m, 3H), 7.38-7.04 (m, 7H), 5.31 (br. s, 1H), 4.04-3.90 (m, 3H), 3.27-3.16 (m, 1H), 2.95-2.83 (m, 1H). MS (ESI) m/z (M+H)⁺ 435.2.

### Example 13—Compounds 20 and 21

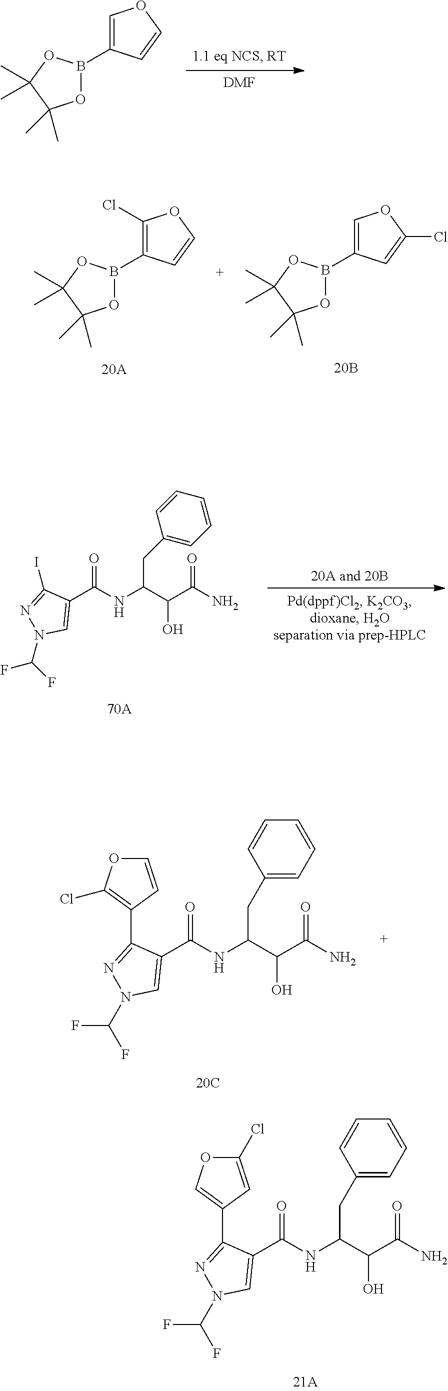

To a solution of 2-(furan-3-yl)-4,4,5,5-tetramethyl-1,3,2-dioxaborolane (1 g, 5.15 mmol) in DMF (15 mL) was added NCS (723 mg, 5.41 mmol). The mixture was stirred at 25° C. for 4 h. resultant solution was treated with 10% Na₂S₂O₃ aqueous (50 mL) and was extracted with MTBE (50 mL×3). The combined organic phase was washed with brine (100 mL) and dried over Na₂SO₄. After removal of solvent under reduced pressure, the residue was purified by flash silica gel chromatography (ISCO®; 12 g SepaFlash® Silica Flash Column, Eluent of 0-10% Ethyl acetate/Petroleum ethergradient @ 25 mL/min) Compound 20A (0.37 g, yield: 31.4%) was obtained as a colorless oil. Compound 20B (0.13 g, yield: 11.0%) was obtained as a colorless oil. The mixture of compound 20A and compound 20B. ¹H NMR (400 MHz, CDCl₃) δ 7.92 (s, 1H), 7.34 (d, J=2.0 Hz, 1H), 6.78 (d, J=2.0 Hz, 1H), 4.23 (q, J=7.1 Hz, 2H), 3.93 (s, 3H), 1.27 (t, J=7.2 Hz, 3H). MS (ESI) m/z (M+H)⁺ 254.9.

To a solution of compound 70A (400 mg, 861.69 umol) and compound 20A (216 mg, 945.38 umol) and compound 20B (80 mg, 350.14 umol) in dioxane (20 mL) and H₂O (2 mL) was added Pd(dppf)Cl₂ (70 mg, 95.67 umol) and K₂CO₃ (300 mg, 2.17 mmol) under N₂, and the mixture was stirred at 90° C. for 16 h under N₂ atmosphere. The reaction mixture was concentrated and the residue was diluted with EA (30 mL) and H₂O (40 mL), filtered, the filtrate was extracted with EA (20 mL×2), and then the organic phase was dried over Na₂SO₄, filtered and concentrated to give a residue. The residue was purified by preparatory-TLC (SiO₂, PE:EA=1:2.5). Then the residue was purified by preparatory-HPLC (HCl condition; column: YMC-Actus Triart C18 100*30 mm*5 um; mobile phase: [water(0.05% HCl)-ACN]; B %: 30%-60%, 10 min). Compound 20C (120 mg, yield: 31.6%) was obtained as a white solid. Compound 21A (45 mg, yield: 11.8%) was obtained as a white solid.

Compound 20C: ¹H NMR (400 MHz, DMSO-d₆) δ 8.61 (s, 0.3H), 8.54 (s, 0.7H), 8.21-7.71 (m, 2H), 7.69-7.62 (m, 1H), 7.31 (d, J=8.4 Hz, 1H), 7.25-7.09 (m, 6H), 6.65-6.57 (m, 1H), 5.86 (d, J=5.7 Hz, 0.7H), 5.75 (d, J=5.7 Hz, 0.3H), 4.50-4.36 (m, 1H), 4.03-3.96 (m, 0.7H), 3.87-3.83 (m, 0.3H), 2.91-2.69 (m, 2H). MS (ESI) m/z (M+H)⁺ 439.0.

Compound 21A: ¹H NMR (400 MHz, DMSO-d₆) δ 8.65 (s, 0.2H), 8.62 (s, 0.8H), 8.23-7.69 (m, 3H), 7.32 (d, J=7.7 Hz, 1H), 7.26-7.08 (m, 6H), 6.71-6.66 (m, 1H), 5.86 (d, J=5.7 Hz, 0.8H), 5.74 (d, J=6.0 Hz, 0.2H), 4.54-4.41 (m, 1H), 4.01 (dd, J=3.5, 5.7 Hz, 0.8H), 3.88 (d, J=5.3 Hz, 0.2H), 2.92-2.67 (m, 2H). MS (ESI) m/z (M+H)⁺439.0.

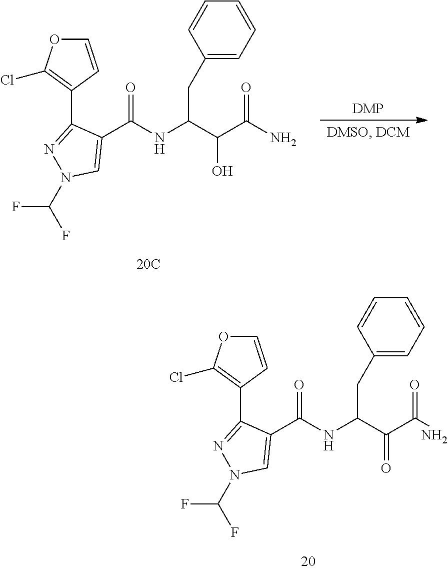

### N-(4-amino-3,4-dioxo-1-phenylbutan-2-yl)-3-(2-chlorofuran-3-yl)-1-(difluoromethyl)-1H-pyrazole-4-carboxamide (20)

Compounds 20C was converted to compound 20 using procedures as described in Example 1. Compound 20 (90 mg, yield: 70.6%) as a white solid was obtained. ¹H NMR (400 MHz, DMSO-d₆) δ 8.73 (d, J=7.5 Hz, 1H), 8.58 (s, 1H), 8.13-7.71 (m, 3H), 7.67 (d, J=2.2 Hz, 1H), 7.30-7.22 (m, 4H), 7.21-7.14 (m, 1H), 6.66 (d, J=2.2 Hz, 1H), 5.38-5.21 (m, 1H), 3.15 (dd, J=3.7, 13.9 Hz, 1H), 2.77 (dd, J=10.0, 13.8 Hz, 1H). MS (ESI) m/z (M+H)⁺ 437.0.

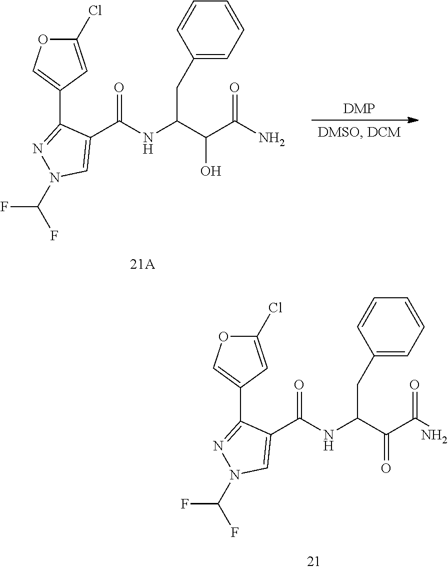

### N-(4-amino-3,4-dioxo-1-phenylbutan-2-yl)-3-(5-chlorofuran-3-yl)-1-(difluoromethyl)-1H-pyrazole-4-carboxamide (21)

Compounds 21A was converted to compound 21 using procedures as described in Example 1. Compound 21 (30 mg, yield: 65.7%) as a white solid was obtained. ¹H NMR (400 MHz, DMSO-d₆) δ 8.81 (d, J=7.5 Hz, 1H), 8.62 (s, 1H), 8.16 (d, J=0.9 Hz, 1H), 8.10 (s, 1H), 8.03-7.71 (m, 2H), 7.26 (d, J=4.2 Hz, 4H), 7.20-7.16 (m, 1H), 6.74 (d, J=0.9 Hz, 1H), 5.36-5.23 (m, 1H), 3.17 (dd, J=3.9, 14.0 Hz, 1H), 2.80 (dd, J=10.3, 14.0 Hz, 1H). MS (ESI) m/z (M+H)⁺437.1.

### Example 14—Compound 36

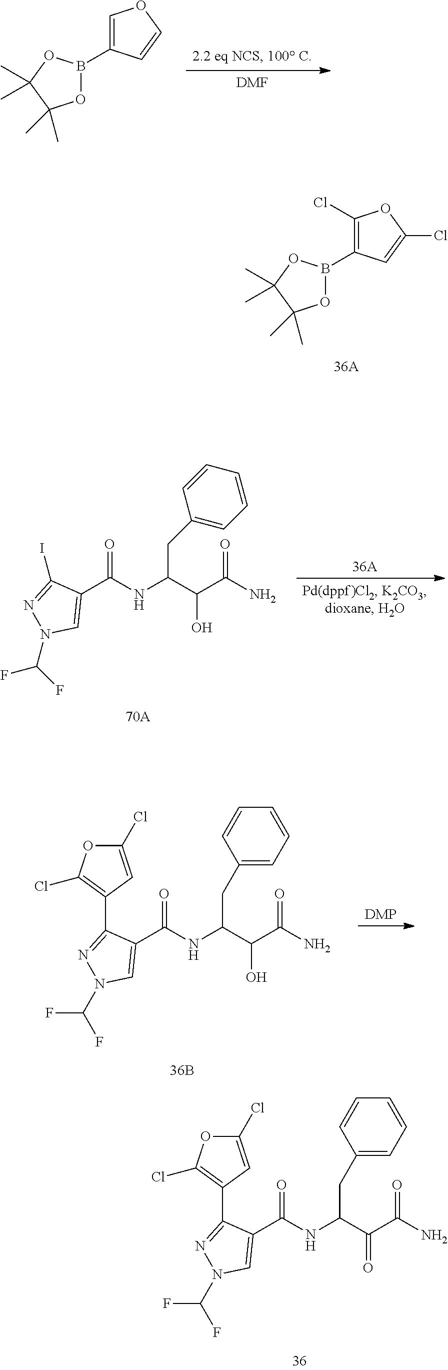

To a solution of 2-(furan-3-yl)-4,4,5,5-tetramethyl-1,3,2-dioxaborolane (1 g, 5.10 mmol) in DMF (15 mL) was added NCS (1.50 g, 11.21 mmol). The mixture was stirred at 100° C. for 2 h. The resultant solution was treated with aq 10% Na₂S₂O₃ (50 mL) and was extracted with MTBE (50 mL×3). The combined organic phase was washed with brine (100 mL) and dried over Na₂SO₄. After removal of solvent under reduced pressure, the residue was purified by flash silica gel chromatography (ISCO®; 12 g SepaFlash® Silica Flash Column, Eluent of 0-10% Ethyl acetate/Petroleum ethergradient @20 mL/min). Compound 36A (0.5 g, yield: 37.0%) was obtained as a yellow oil. ¹H NMR (400 MHz, CDCl₃) δ 6.45-6.23 (m, 1H), 1.31 (s, 12H).

### N-(4-amino-3,4-dioxo-1-phenylbutan-2-yl)-3-(2,5-dichlorofuran-3-yl)-1-(difluoromethyl)-1H-pyrazole-4-carboxamide (36)

Compounds 36A and intermediate 70A (prepared from 4A using same procedure as 17B) were converted to compound 36 using procedures as described in Example 1. Compound 36 (100 mg, yield: 71.7%) as a white solid was obtained. ¹H NMR (400 MHz, DMSO-d₆) δ 8.78 (d, J=7.5 Hz, 1H), 8.65 (s, 1H), 8.16-7.72 (m, 3H), 7.32-7.22 (m, 4H), 7.21-7.12 (m, 1H), 6.67 (s, 1H), 5.47-5.19 (m, 1H), 3.15 (dd, J=3.6, 13.8 Hz, 1H), 2.76 (dd, J=10.1, 13.9 Hz, 1H). MS (ESI) m/z (M+H)⁺471.0.

### Example 15—Compounds 19 and 15

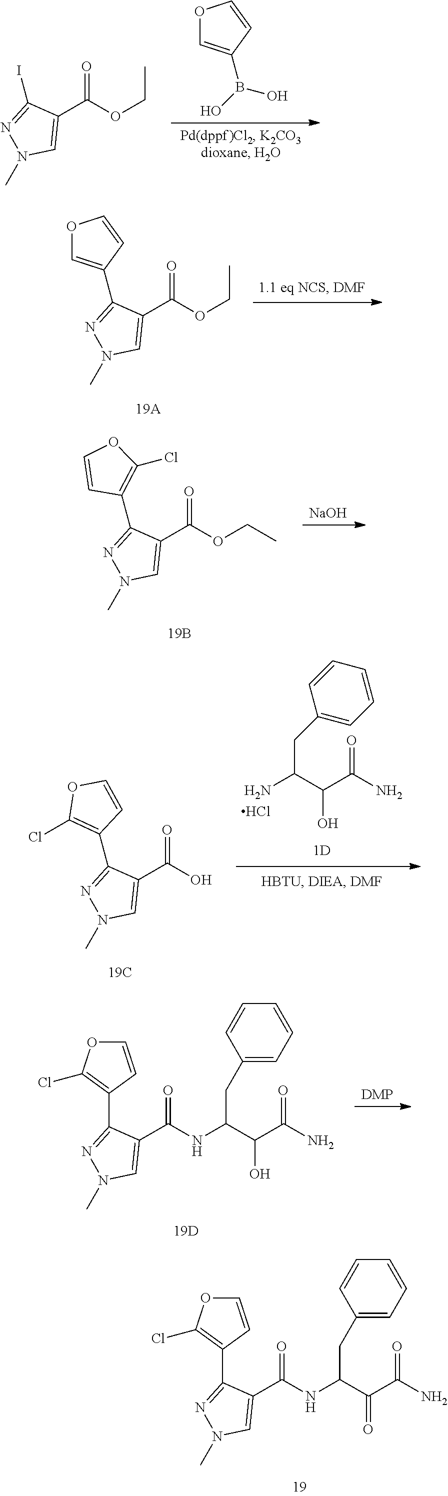

To a solution of 2-(furan-3-yl)-4,4,5,5-tetramethyl-1,3,2-dioxaborolane (500 mg, 1.79 mmol) and 3-furylboronic acid (250 mg, 2.23 mmol) in dioxane (20 mL) and H₂O (1 mL) was added K₂CO₃ (620 mg, 4.49 mmol) and Pd(dppf)Cl₂ (131 mg, 179.03 umol) under N₂. The mixture was stirred at 80° C. for 16 h under N₂. The reaction mixture was concentrated and the residue was diluted with EA (30 mL) and H₂O (30 mL), filtered. The filtrate was extracted with EA (20 mL), and then the organic phase was dried over Na₂SO₄, filtered and concentrated to give a residue. The residue was purified by flash silica gel chromatography (ISCO®; 24 g SepaFlash® Silica Flash Column, Eluent of 0-30% Ethyl acetate/Petroleum ethergradient @ 30 mL/min) Compound 19A (350 mg, yield: 88.8%) was obtained as a light yellow oil. ¹H NMR (400 MHz, CDCl₃) δ 8.39 (s, 1H), 7.91 (s, 1H), 7.44 (t, J=1.6 Hz, 1H), 6.95 (d, J=1.3 Hz, 1H), 4.30 (q, J=7.0 Hz, 2H), 3.92 (s, 3H), 1.35 (t, J=7.2 Hz, 3H). MS (ESI) m/z (M+H)⁺ 221.0.

To a solution of compound 19A (100 mg, 454.08 umol) in DMF (3 mL) was added NCS (68 mg, 509.24 umol). The mixture was stirred at 25° C. for 2 h. The reaction was diluted with H₂O (20 mL), extracted with EA (20 mL×2), the organic phase was dried over Na₂SO₄, filtered, and concentrated to give a residue. The residue was purified by preparatory-TLC (SiO₂, PE:EA=2:1). Compound 19B (70 mg, yield: 60.5%) was obtained as a white solid. ¹H NMR (400 MHz, CDCl₃) δ 7.92 (s, 1H), 7.34 (d, J=2.0 Hz, 1H), 6.78 (d, J=2.0 Hz, 1H), 4.23 (q, J=7.1 Hz, 2H), 3.93 (s, 3H), 1.27 (t, J=7.2 Hz, 3H). MS (ESI) m/z (M+H)⁺ 254.9.

### N-(4-amino-3,4-dioxo-1-phenylbutan-2-yl)-3-(2-chlorofuran-3-yl)-1-methyl-1H-pyrazole-4-carboxamide (19)

Compounds 19B was converted to compound 19 using procedures as described in Example 1. Compound 19 (40 mg, yield: 35.0%) as a white solid was obtained. ¹H NMR (400 MHz, DMSO-d₆) δ 8.32 (d, J=7.5 Hz, 1H), 8.14 (s, 1H), 8.06 (s, 1H), 7.80 (s, 1H), 7.64 (d, J=2.0 Hz, 1H), 7.32-7.24 (m, 4H), 7.23-7.19 (m, 1H), 6.66 (d, J=2.0 Hz, 1H), 5.33-5.25 (m, 1H), 3.89 (s, 3H), 3.15 (dd, J=3.9, 13.9 Hz, 1H), 2.82 (dd, J=9.9, 13.9 Hz, 1H). MS (ESI) m/z (M+H)⁺ 401.1.

### N-(4-amino-3,4-dioxo-1-phenylbutan-2-yl)-3-(2,5-dichlorofuran-3-yl)-1-methyl-1H-pyrazole-4-carboxamide (15)

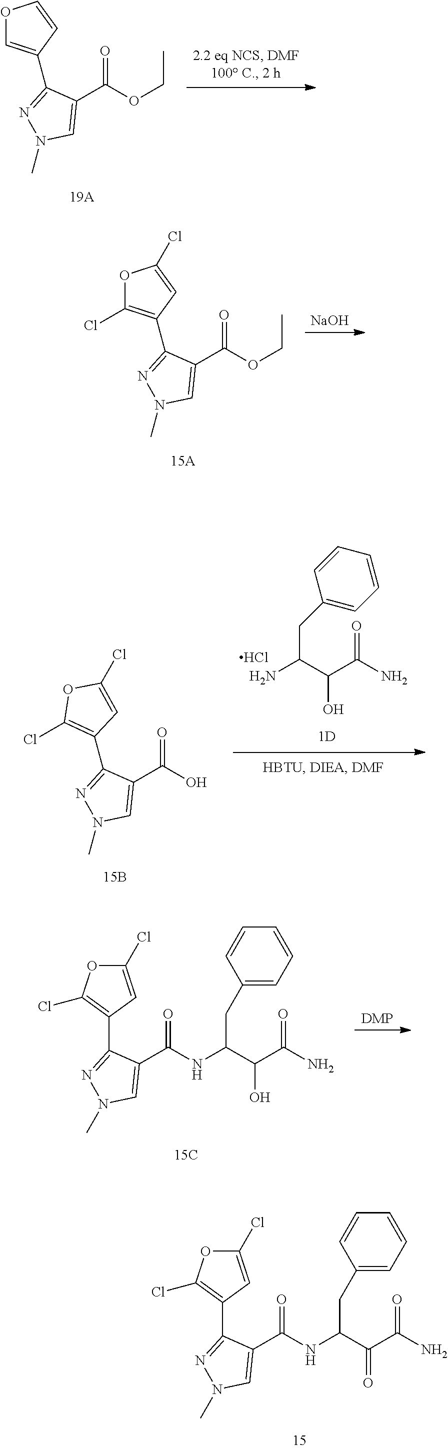

To a solution of compound 19A (50 mg, 227.04 umol) in DMF (2 mL) was added NCS (68 mg, 509.24 umol). The mixture was stirred at 100° C. for 1.5 h. The reaction was diluted with H₂O (20 mL), extracted with EA (20 mL×2), the organic phase was dried over Na₂SO₄, filtered, and concentrated to give a residue. The residue was purified by preparatory-TLC (SiO₂, PE:EA=2:1). Compound 15A (40 mg, yield 60.9%) was obtained as a white solid. ¹H NMR (400 MHz, CDCl₃) δ 7.93 (s, 1H), 6.63 (s, 1H), 4.34-4.18 (m, 2H), 3.95 (s, 3H), 1.31 (t, J=7.2 Hz, 3H). MS (ESI) m/z (M+H)⁺ 289.0.

Compounds 15A was converted to compound 15 using procedures as described in Example 1. Compound 15 (35 mg, yield: 47.2%) as a white solid was obtained. ¹H NMR (400 MHz, DMSO-d₆) δ 8.40 (d, J=7.5 Hz, 1H), 8.15 (s, 1H), 8.05 (s, 1H), 7.77 (s, 1H), 7.29-7.21 (m, 4H), 7.20-7.15 (m, 1H), 6.63 (s, 1H), 5.35-5.19 (m, 1H), 3.86 (s, 3H), 3.12 (dd, J=3.7, 13.9 Hz, 1H), 2.78 (dd, J=10.1, 13.9 Hz, 1H). MS (ESI) m/z (M+H)⁺ 435.0.

### Example 16—Compounds 23, 3, 46, 52, and 79

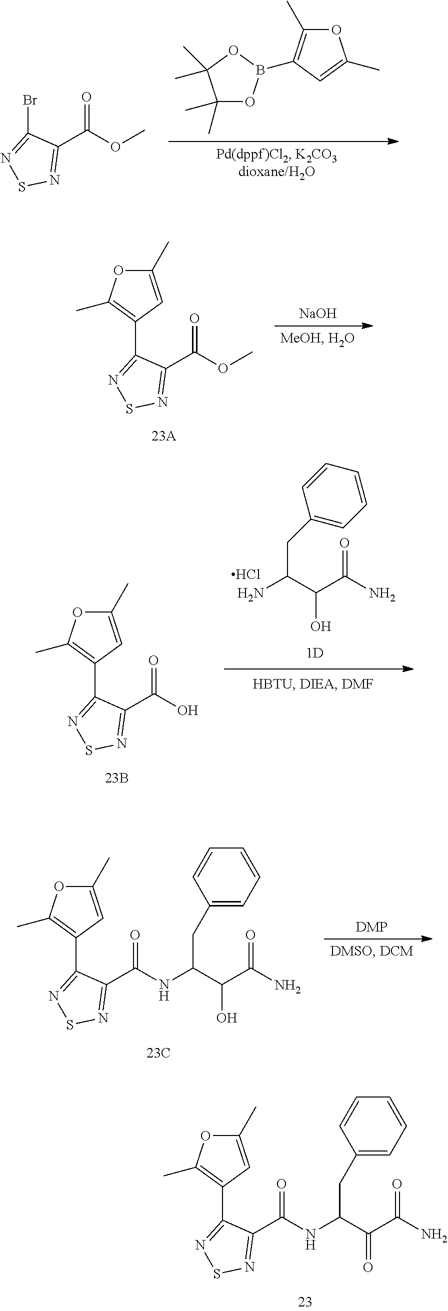

### N-(4-amino-3,4-dioxo-1-phenylbutan-2-yl)-4-(2,5-dimethylfuran-3-yl)-1,2,5-thiadiazole-3-carboxamide (23)

Compounds methyl 4-bromo-1,2,5-thiadiazole-3-carboxylate and 2-(2,5-dimethylfuran-3-yl)-4,4,5,5-tetramethyl-1,3,2-dioxaborolane was converted to compound 23 using procedures as described in Example 1. Compound 23 (110 mg, yield: 65.02%) as a white solid was obtained. ¹H NMR (400 MHz, DMSO-d₆) δ 9.34 (d, J=7.9 Hz, 1H), 8.21 (s, 1H), 7.93 (s, 1H), 7.37-7.18 (m, 5H), 5.94 (s, 1H), 5.61-5.41 (m, 1H), 3.23 (dd, J=3.5, 14.1 Hz, 1H), 2.85 (dd, J=10.0, 14.0 Hz, 1H), 2.37 (s, 3H), 2.18 (s, 3H). MS (ESI) m/z (M+H)⁺ 399.1.

### N-(4-amino-3,4-dioxo-1-phenylbutan-2-yl)-4-(4-fluorophenyl)-1,2,5-thiadiazole-3-carboxamide (3)

Compounds methyl 4-bromo-1,2,5-thiadiazole-3-carboxylate and (4-fluorophenyl)boronic acid was converted to compound 3 using procedures as described in Example 1. Compound 3 (235 mg, yield: 68.1%) as a white solid was obtained. ¹H NMR (400 MHz, DMSO-d₆) δ 9.43 (d, J=7.7 Hz, 1H), 8.26-8.12 (m, 1H), 7.93 (s, 1H), 7.67-7.56 (m, 2H), 7.34-7.16 (m, 7H), 5.56-5.38 (m, 1H), 3.24 (dd, J=3.6, 14.0 Hz, 1H), 2.86 (dd, J=10.3, 14.0 Hz, 1H). MS (ESI) m/z (M+H)⁺399.1.

### N-(4-amino-3,4-dioxo-1-phenylbutan-2-yl)-4-(2-Methylfuran-3-yl)-1,2,5-thiadiazole-3-carboxamide (46)

Compounds ethyl 4-chloro-1,2,5-thiadiazole-3-carboxylate and 4,4,5,5-tetramethyl-2-(2-methylfuran-3-yl)-1,3,2-dioxaborolane was converted to compound 46 using procedures as described in Example 1. Compound 46 (45 mg, yield: 42.84%) as a pale yellow solid was obtained. ¹H NMR (400 MHz, DMSO-d₆) δ 9.34 (d, J=7.7 Hz, 1H), 8.20 (s, 1H), 7.92 (s, 1H), 7.46 (d, J=2.0 Hz, 1H), 7.32-7.25 (m, 4H), 7.22 (qd, J=4.1, 8.7 Hz, 1H), 6.35 (d, J=2.0 Hz, 1H), 5.60-5.43 (m, 1H), 3.22 (dd, J=3.5, 13.9 Hz, 1H), 2.85 (dd, J=10.1, 14.1 Hz, 1H), 2.40 (s, 3H). MS (ESI) m/z (M+H)⁺385.1.

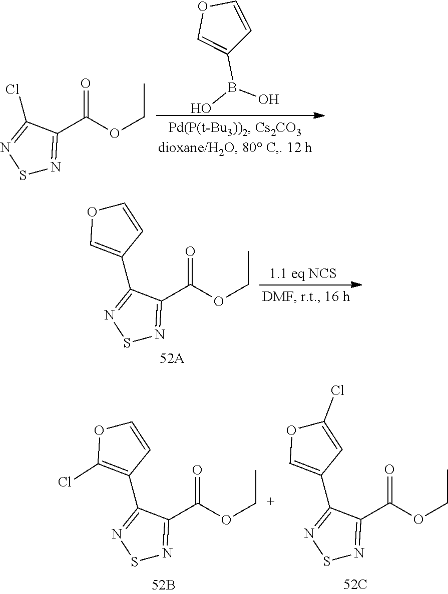

To a solution of ethyl 4-chloro-1,2,5-thiadiazole-3-carboxylate (3.0 g, 15.57 mmol) in dioxane (50 mL) and H₂O (5 mL) was added Cs₂CO₃ (15.2 g, 46.72 mmol) and 3-furylboronic acid (2.1 g, 18.69 mmol), the mixture was degassed and purged with N₂ for 3 times, then Pd(P(t-Bu)₃)₂ (796 mg, 1.56 mmol) was added. The mixture was stirred at 80° C. for 12 hours under N₂ and cooled to room temperature and concentrated, the residue was diluted with H₂O (100 mL) and extracted with EA (100 mL×3). The obtained organic phase was combined, washed with brine (50 mL×3) and dried over anhydrous Na₂SO₄ and filtered and the filtrate was concentrated to give a residue, which was purified by silica gel column chromatography (PE:EA=1:0 to 10:1) to give compound 52A (2 g, yield 57.3%) as a colorless oil. ¹H NMR (CDCl₃, 400 MHz) δ 8.44 (s, 1H), 7.51 (d, J=1.6 Hz, 1H), 7.03 (d, J=1.6 Hz, 1H), 4.50 (q, J=6.8 Hz, 2H), 1.49 (t, J=6.8 Hz, 3H).

To a solution of compound 52A (1.5 g, 6.69 mmol) in DMF (20 mL) was added NCS (1.0 g, 7.49 mmol). The mixture was stirred at 25° C. for 16 hours. The reaction was diluted with H₂O (60 mL) and extracted with EA (20 mL×3), the combined organic phase was washed with Na₂S₂O₃ (10% aq., 20 mL) and brine (20 mL×3) and concentrated to give a residue. The residue was purified by silica gel column chromatography (PE:EA=1:0 to 10:1) to give pure compound 52B (330 mg, yield: 19.5%) as a colorless oil and the mixture consist of compound 52A and compound 52C (500 mg). The mixture consist of compound 52A and compound 52C was purified by preparatory-TLC (PE:EA=100:1, 5 times) to give compound 52C (135 mg, yield: 7.8%) as a white solid. Compound 52B: ¹H NMR (CDCl₃, 400 MHz) δ 7.43 (d, J=1.6 Hz, 1H), 6.85 (d, J=1.6 Hz, 1H), 4.60 (q, J=7.2 Hz, 2H), 1.42 (t, J=7.2 Hz, 3H). Compound 52C: ¹H NMR (CDCl₃, 400 MHz) δ 8.35 (s, 1H), 6.85 (s, 1H), 4.50 (q, J=7.2 Hz, 2H), 1.48 (t, J=7.2 Hz, 3H).

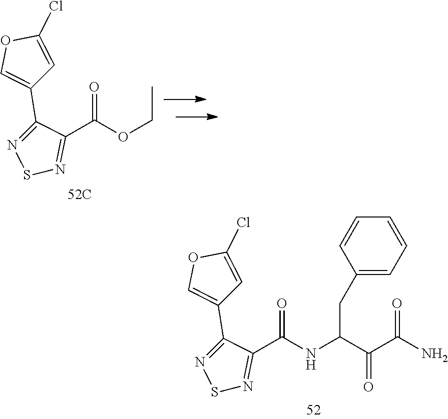 [SMILES: CCOC(=O)c1nsnc1-c1coc(Cl)c1 ; NC(=O)C(=O)C(Cc1ccccc1)NC(=O)c1nsnc1-c1coc(Cl)c1]

### N-(4-amino-3,4-dioxo-1-phenylbutan-2-yl)-4-(5-chlorofuran-3-yl)-1,2,5-thiadiazole-3-carboxamide (52)

Compounds ethyl 4-(5-chlorofuran-3-yl)-1,2,5-thiadiazole-3-carboxylate (52C) was converted to compound 52 using procedures as described in Example 1. Compound 52 (60 mg, yield: 62.8%) as a white solid was obtained. ¹H NMR (400 MHz, DMSO-d₆) δ 9.37 (d, J=7.7 Hz, 1H), 8.22 (s, 1H), 8.06 (d, J=1.1 Hz, 1H), 7.93 (s, 1H), 7.32-7.18 (m, 5H), 6.81 (d, J=1.1 Hz, 1H), 5.57-5.49 (m, 1H), 3.25 (dd, J=3.9, 14.0 Hz, 1H), 2.89 (dd, J=10.3, 14.0 Hz, 1H). MS (ESI) m/z (M+H)⁺ 405.0.

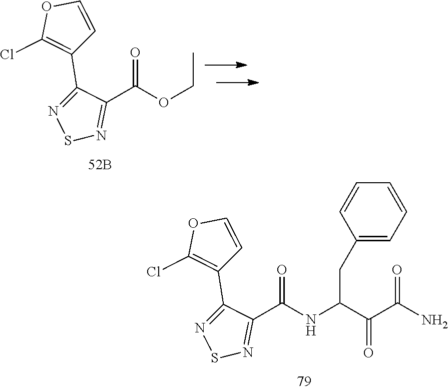 [SMILES: CCOC(=O)c1nsnc1-c1ccoc1Cl ; NC(=O)C(=O)C(Cc1ccccc1)NC(=O)c1nsnc1-c1ccoc1Cl]

### N-(4-amino-3,4-dioxo-1-phenylbutan-2-yl)-4-(2-chlorofuran-3-yl)-1,2,5-thiadiazole-3-carboxamide (79)

Compounds ethyl 4-(2-chlorofuran-3-yl)-1,2,5-thiadiazole-3-carboxylate (52B) was converted to compound 79 using procedures as described in Example 1. Compound 52 (50 mg, yield: 52.3%) as a pale yellow solid was obtained. ¹H NMR (400 MHz, DMSO-d₆) δ 9.37 (d, J=7.7 Hz, 1H), 8.20 (s, 1H), 7.92 (s, 1H), 7.73 (d, J=2.2 Hz, 1H), 7.32-7.17 (m, 5H), 6.59 (d, J=2.2 Hz, 1H), 5.56-5.47 (m, 1H), 3.29-3.18 (m, 1H), 2.88 (dd, J=10.0, 14.0 Hz, 1H). MS (ESI) m/z (M+H)⁺ 405.0.

### Example 17—Compounds 85-86, 57, and 82

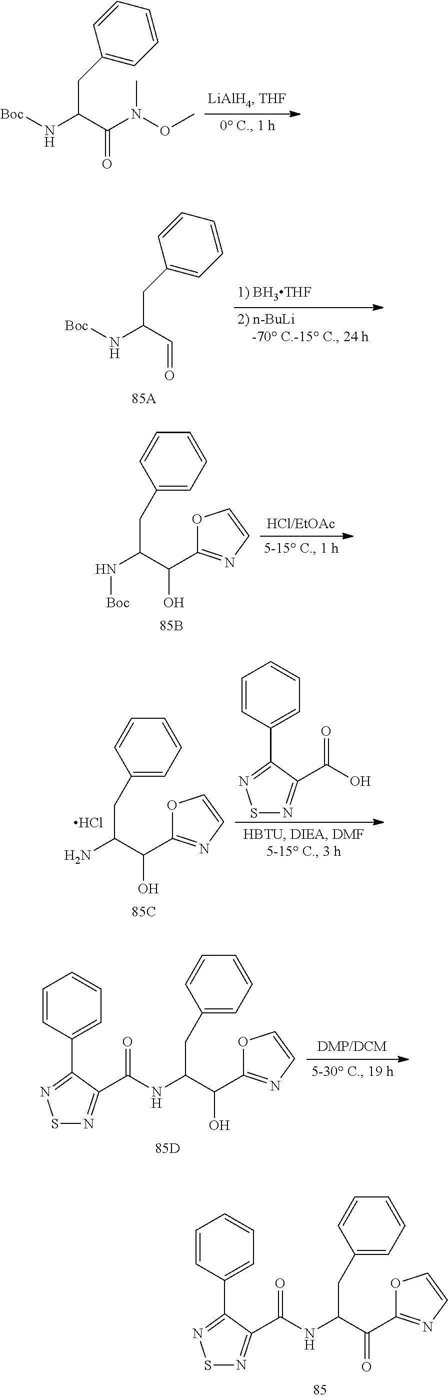

### N-(1-(oxazol-2-yl)-1-oxo-3-phenylpropan-2-yl)-4-phenyl-1,2,5-thiadiazole-3-carboxamide (85)

To the mixture of LiAlH₄ (406.2 mg, 10.70 mmol) in THF (20 mL), solution of tert-butyl (1-(methoxy(methyl)amino)-1-oxo-3-phenylpropan-2-yl)carbamate (3 g, 9.73 mmol) in THF (20 mL) was added drop-wise at 0° C. under N₂ atmosphere. After addition, the mixture was stirred at 0° C. for 1 h. EtOAc (6 mL) was added drop-wise to the reaction mixture maintaining the temperature below 5° C., after that HCl (1M, 10 mL) was added. The reaction mixture was separated in a separation funnel and the aqueous was extracted with EtOAc (30 mL×2), the combined organic phase was washed with HCl (1M, 30 mL×3), sat. NaHCO₃ (30 mL) and brine (30 mL), dried over anhydrous Na₂SO₄. Filtered and the filtrate was concentrated to give compound 85A (2.3 g, yield: 94.8%) as a white solid. The product was used directly in next step. ¹H NMR (400 MHz, DMSO-d₆) δ 9.52 (s, 1H), 7.40-7.10 (m, 6H), 4.15-4.00 (m, 1H), 3.13-3.05 (m, 1H), 2.75-2.65 (m, 1H), 1.31 (s, 9H).

A solution comprised of oxazole (166.2 mg, 2.41 mmol) in THF (20 mL) was treated with BH₃.THF (1 M, 2.41 mL) under nitrogen and the mixture was stirred at 5-15° C. for 30 minutes and then cooled to −70° C. A solution comprised of n-BuLi (2.5M in cyclohexane, 1 mL) was added drop-wise and the mixture was stirred for 30 minutes at −70° C. A solution comprised of compound 85A (300 mg, 1.20 mmol) in THF (10 mL) was added and the mixture was stirred and allowed to warm to room temperature (5-15° C.) while the reaction proceeded to completion (24 h after). The mixture then was cooled to −78° C., quenched by slowly adding 5 percent acetic acid in ethanol (13.8 mL), allowed to warm to ambient temperature (5-15° C.) and stirred for 18 hours. The solvent was removed under reduced pressure, the residue was diluted with H₂O (15 mL) and extracted with EtOAc (20 mL×3). The organic phase was combined, washed with brine (30 mL) and concentrated to give a residue. The residue was purified by silica gel column chromatography (PE:EA=1:0 to 0:1) to give compound 85B (170 mg, yield: 24.4%) as a colorless oil. MS (ESI) m/z (M-Boc)⁺218.9.

The mixture of compound 85B (170 mg, 533.97 umol) in EtOAc (5 mL) was mixed with HCl/EtOAc (4M, 10 mL) and stirred at room temperature (5-15° C.) for 1 h. The solvent was removed under reduced pressure to give compound 85C (150 mg, crude, HCl) as a white solid. The product was used directly in next step.

The mixture of 4-phenyl-1,2,5-thiadiazole-3-carboxylic acid (121.4 mg, 588.9 umol), compound 85C (150 mg, 588.90 umol, HCl), DIEA (0.3 mL, 1.77 mmol) and HBTU (245.67 mg, 647.79 umol) in DMF (10 mL) was stirred at 5-15° C. for 3 h. The reaction was diluted with H₂O (30 mL), extracted with EtOAc (30 mL×3). The organic phase was combined and washed with HCl (1M, 30 mL), sat. NaHCO₃aq. (30 mL), brine (30 mL×2) and concentrated to give a residue. The residue was purified by preparatory-HPLC (HCl system) purification to give compound 85D (50 mg, yield: 20.8%) as a white solid. ¹H NMR (400 MHz, DMSO-d₆) δ 9.02-8.83 (d, J=7.7 Hz, 1H), 8.07 (s, 1H), 7.52-7.16 (m, 12H), 4.88-4.74 (m, 1H), 4.64-4.49 (m, 1H), 3.20-2.77 (m, 2H). MS (ESI) m/z (M+H)⁺ 407.0.

To the mixture of compound 85D (50 mg, 123.01 umol) in DCM (20 mL), DMP (156.5 mg, 369.04 umol) was added and stirred at room temperature (5-15° C.). After 1.5 h, DMP (100 mg) was added and the reaction was stirred at 30° C. overnight (16 h). The reaction was diluted with DCM (20 mL), quenched with sat. Na₂S₂O₃ aqueous (30 mL) and separated. The organic phase was washed with sat. NaHCO₃ aqueous (20 mL) and brine (20 mL×3), dried over anhydrous Na₂SO₄. Filtered and the filtrate was concentrated. Compound 85 (40 mg, yield: 62.3%) was obtained as a pale yellow solid. ¹H NMR (400 MHz, DMSO-d₆) δ 9.68 (d, J=7.6 Hz, 1H), 8.50 (s, 1H), 7.66 (s, 1H), 7.58-7.52 (m, 2H), 7.49-7.42 (m, 1H), 7.41-7.22 (m, 7H), 5.74-5.66 (m, 1H), 3.41-3.36 (m, 1H), 3.06-2.95 (m, 1H). MS (ESI) m/z (M+H)⁺ 405.1. ¹H NMR (400 MHz, CDCl₃) δ 7.88 (s, 1H), 7.73-7.66 (m, 3H), 7.47-7.38 (m, 4H), 7.32-7.22 (m, 3H), 7.19-7.13 (m, 2H), 5.99 (dt, J=5.3, 7.8 Hz, 1H), 3.52 (dd, J=5.1, 13.9 Hz, 1H), 3.26 (dd, J=7.5, 14.1 Hz, 1H).

### N-(1-(benzo[d]oxazol-2-yl)-1-oxo-3-phenylpropan-2-yl)-4-phenyl-1,2,5-thiadiazole-3-carboxamide (86)

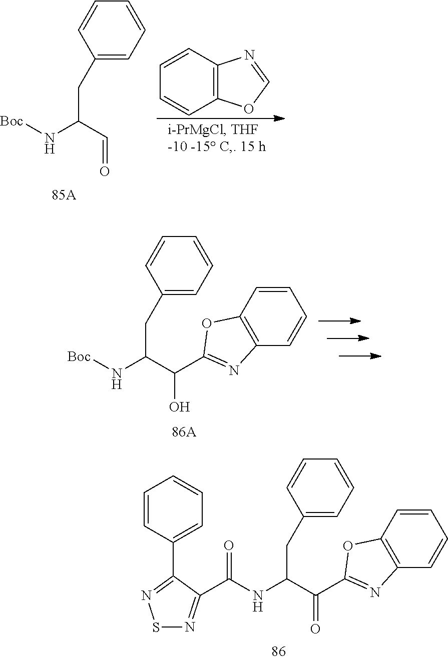

To a solution of 1, 3-benzoxazole (573.4 mg, 4.81 mmol) in THF (20 mL) at −10° C. was added i-PrMgCl (2.0 M, 1.60 mL), the reaction mixture was stirred at −10° C. for 1 h. Then compound 85A (400 mg, 1.60 mmol) was added as a solution in THF (20 mL) and the reaction mixture was stirred at −10° C. for 2 h followed by 12 h at 5-15° C. The reaction was concentrated and the residue was diluted with EtOAc (60 mL), washed with brine (30 mL×2) and concentrated to give a residue. The residue was diluted with EtOAc (100 mL) and washed with brine (30 mL×3), concentrated to give the crude product. The crude product was purified by silica gel column chromatography (PE:EA=1:0 to 5:1) to give compound 86A (270 mg, yield: 45%) as a yellow oil. ¹H NMR (400 MHz, CDCl₃) δ 7.77-7.63 (m, 1H), 7.52 (dt, J=2.6, 6.7 Hz, 1H), 7.41-7.30 (m, 4H), 7.26-7.13 (m, 3H), 5.11-4.88 (m, 2H), 4.53-4.19 (m, 2H), 3.08 (br. d, J=7.6 Hz, 1H), 3.00-2.83 (m, 1H), 1.43-1.27 (m, 9H). MS (ESI) m/z (M+Na⁺) 391.0.

Compound 86A was converted to compound 86 using procedures as described as for compound 85. Compound 86 (180 mg, yield: 78.53%) as a white solid was obtained. ¹H NMR (400 MHz, DMSO-d₆) δ 9.78 (d, J=7.3 Hz, 1H), 8.04 (d, J=8.1 Hz, 1H), 7.94 (d, J=8.3 Hz, 1H), 7.68 (t, J=7.5 Hz, 1H), 7.60-7.52 (m, 3H), 7.46-7.40 (m, 1H), 7.40-7.28 (m, 6H), 7.27-7.21 (m, 1H), 5.89-5.79 (m, 1H), 3.49 (dd, J=3.8, 14.1 Hz, 1H), 3.07 (dd, J=9.9, 14.1 Hz, 1H). MS (ESI) m/z (M+H)⁺ 455.0.

### N-(1-(oxazol-2-ylamino)-1-oxo-3-phenylpropan-2-yl)-4-phenyl-1,2,5-thiadiazole-3-carboxamide (57)

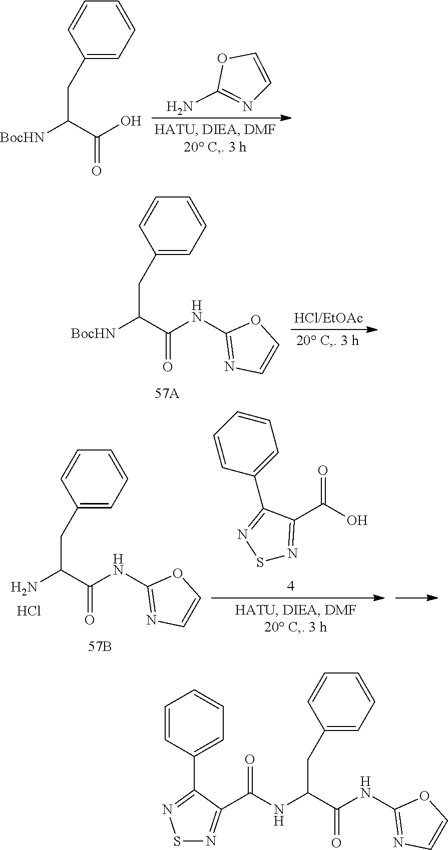

Compounds (tert-butoxycarbonyl)phenylalanine and oxazol-2-amine were coupled using conditions described for compound 85 to yield intermediate 57A which was converted to compound 57. Compound 57 (35 mg, yield: 11.2%) as a white solid was obtained. ¹H NMR (400 MHz, DMSO-d₆) δ 11.72 (br. s, 1H), 9.47 (br. d, J=7.7 Hz, 1H), 7.93 (s, 1H), 7.51 (d, J=7.3 Hz, 2H), 7.46-7.36 (m, 3H), 7.36-7.22 (m, 5H), 7.15 (s, 1H), 5.00-4.80 (m, 1H), 3.25-3.10 (m, 1H), 3.05-2.93 (m, 1H). MS (ESI) m/z (M+H)⁺ 420.2.

### N-(1-cyano-2-phenylethyl)-4-phenyl-1,2,5-thiadiazole-3-carboxamide (82)

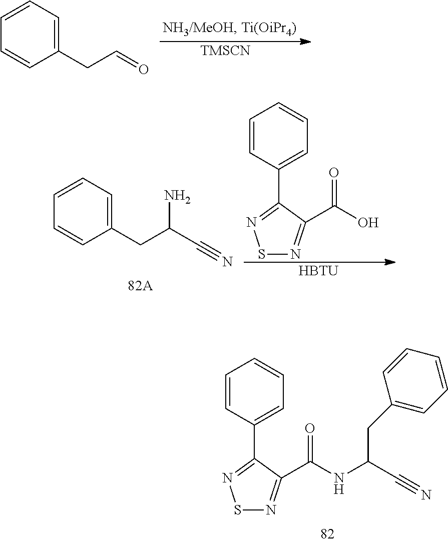

To a stirred solution of 2-phenylacetaldehyde (3 g, 24.97 mmol, 1.95 mL) in MeOH (70 mL) was added NH₃ in MeOH (30 mL) and Ti(i-PrO)₄ (10.64 g, 37.45 mmol, 11.05 mL) and the resulting solution was stirred at 15° C. for 2 h. To the reaction mixture was then added TMSCN (4.46 g, 44.94 mmol, 5.62 mL), then the reaction mixture was stirred at 15° C. for 16 h. Reaction mixture was quenched with water (150 mL), and the resulting white precipitate was filtered. The filtrate was concentrated under reduced pressure, extracted with ethyl acetate (50 mL×3) and the organic phase was washed with brine (100 mL). The organic layer was dried over Na₂SO₄, filtered and concentrated under reduced pressure. Compound 82A (2 g, yield: 54.8%) was obtained as a yellow oil, which was used into the next step without further purification. ¹H NMR (400 MHz, DMSO-d₆) δ 7.36-7.20 (m, 5H), 4.03-3.85 (m, 1H), 3.00-2.80 (m, 2H), 2.38 (br s, 2H)

Compound 82A was coupled with 4-phenyl-1,2,5-thiadiazole-3-carboxylic acid using conditions as described for compound 85 to yield compound 82. Compound 82 (130 mg, yield: 40.1%) as a white solid was obtained. ¹H NMR (400 MHz, DMSO-d₆) δ 9.88 (br d, J=7.8 Hz, 1H), 7.61-7.46 (m, 3H), 7.45-7.39 (m, 2H), 7.38-7.20 (m, 5H), 5.25 (q, J=7.8 Hz, 1H), 3.30-3.07 (m, 2H).

### Example 18—Compounds 41, 40, 38, 67, 40, 65, 42, 64, 74, and 72

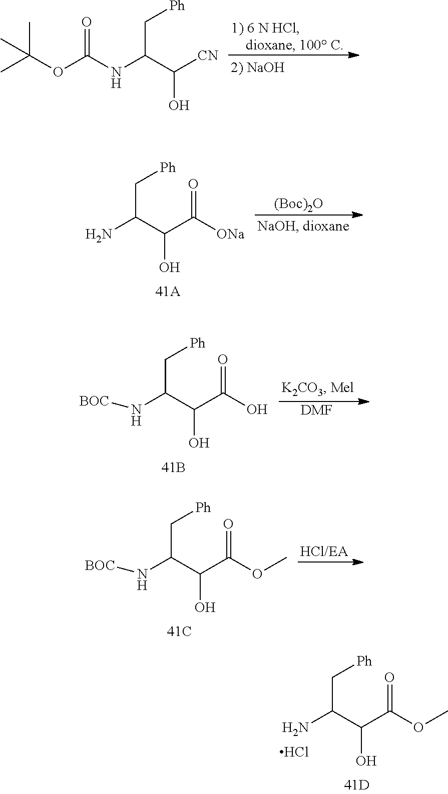

To a mixture of tert-butyl (1-cyano-1-hydroxy-3-phenylpropan-2-yl)carbamate (27 g, 97.7 mmol) in dioxane (150 mL) was added HCl (6 N, 360 mL). The mixture was stirred at 100° C. for 12 h. The hydrolysis reaction was allowed to cool to room temperature and then concentrated to 120 mL in vacuo. The aqueous phase was alkalized with NaOH (solid) till pH ˜11-12. The alkalized aqueous phase was used in next step without purification.

To a mixture of the alkalized aqueous solution compound 41A (97.7 mmol) in H₂O (120 mL)) was added dioxane (60 mL) and (Boc)₂O (45 mL, 195.9 mmol), which was stirred at 25° C. for 12 h while the pH was maintained between 10 and 11 with NaOH (2M). The mixture was concentrated under reduce pressure to move dioxane. After being alkalized to pH ˜12-13, the aqueous phase was washed with EA (80 mL×2) and acidified with 6N HCl till pH ˜2-3, and then extracted with EA (50 mL×3). The combined organic phases were washed with brine (50 mL), dried over Na₂SO₄, filtered and concentrated in vacuo to afford compound 41B (29.5 g, crude) as light red sticky liquid, which was used in next step without purification. ¹H NMR (DMSO-d₆, 400 MHz): δ 7.32-7.14 (m, 6H), 6.73-6.35 (m, 1H), 4.00-3.83 (m, 2H), 2.87-2.75 (m, 1H), 2.74-2.66 (m, 1H), 1.32-1.24 (m, 9H).

To a mixture of compound 41B (11 g, 37.3 mmol) in DMF (80 mL) was added K₂CO₃ (10.3 g, 74.5 mmol), followed by MeI (4.9 mL 78.9 mmol). The mixture was stirred at 25° C. for 2 h. The mixture was filtered. The filtrates was concentrated under reduced pressure and then diluted with H₂O (200 mL) and extracted with EA (50 mL×3). The combined organic phase was washed with brine (50 mL), dried over Na₂SO₄, filtered and concentrated in vacuo to afford compound 41C (8.56 g, 74.2% yield) as light yellow solid, which was used in next step without purification. ¹H NMR (DMSO-d₆, 400 MHz): δ 7.33-7.11 (m, 5H), 6.84-5.99 (m, 1H), 5.91-5.34 (m, 1H), 4.03-3.80 (m, 2H), 3.64-3.52 (m, 3H), 2.86-2.75 (m, 1H), 2.71-2.59 (m, 1H), 1.33-1.15 (m, 9H). MS (ESI) m/z (M+Na)⁺ 332.1, (M-Boc+H)⁺ 210.1.

To a mixture of compound 41C (4 g, 12.9 mmol) in EtOAc (10 mL) was added HCl/EtOAc (4M, 40 mL). The mixture was stirred at 25° C. for 3 h. The mixture was concentrated in vacuo. The residue was triturated with EA (20 mL). The solid was collected and dried in vacuum to afford compound 41D (2.68 g, 84.3% yield, HCl) as white solid. ¹H NMR (DMSO-d₆, 400 MHz): δ 8.27 (s, 3H), 7.41-7.17 (m, 5H), 6.71-6.34 (m, 1H), 4.53-3.93 (m, 1H), 3.77-3.60 (m, 1H), 3.59 (s, 2H), 3.27 (s, 1H), 3.11-2.82 (m, 2H).

### Methyl 3-(1-cyclopropyl-3-phenyl-1H-pyrazole-4-carboxamido)-2-oxo-4-phenylbutanoate (41) and 3-(1-cyclopropyl-3-phenyl-1H-pyrazole-4-carboxamido)-2-oxo-4-phenylbutanoic Acid (60)

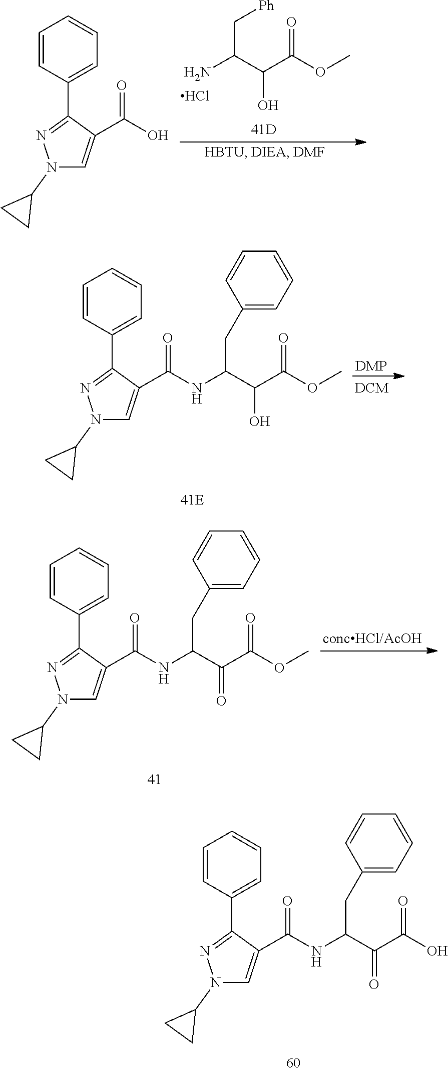 [SMILES: O=C(O)c1cn(C2CC2)nc1-c1ccccc1 ; Cl ; COC(=O)C(O)C(N)Cc1ccccc1 ; COC(=O)C(O)C(Cc1ccccc1)NC(=O)c1cn(C2CC2)nc1-c1ccccc1 ; COC(=O)C(=O)C(Cc1ccccc1)NC(=O)c1cn(C2CC2)nc1-c1ccccc1 ; O=C(O)C(=O)C(Cc1ccccc1)NC(=O)c1cn(C2CC2)nc1-c1ccccc1]

To a mixture of 1-cyclopropyl-3-phenyl-1H-pyrazole-4-carboxylic acid (0.3 g, 1.3 mmol) and intermediate 41D (387.5 mg, 1.6 mmol, HCl) in DMF (10 mL) was added HBTU (500 mg, 1.3 mmol) and DIEA (750 uL, 4.31 mmol). The mixture was stirred at 25° C. for 1 h. The mixture was concentrated, and then diluted with H₂O (100 mL) and extracted with EA (30 mL×3). The combined organic phase was washed with 1N HCl (30 mL), saturated NaHCO₃ (30 mL), brine (30 mL×3), dried over Na₂SO₄, filtered and concentrated in vacuo to afford compound 41E (0.55 g, 99.7% yield) as white solid, which was used in next step without purification. ¹H NMR (DMSO-d₆, 400 MHz): δ 8.10-7.99 (m, 1H), 7.96-7.67 (m, 1H), 7.57-7.45 (m, 2H), 7.33-7.13 (m, 8H), 5.96-5.55 (m, 1H), 4.52-4.33 (m, 1H), 4.16-4.07 (m, 1H), 3.83-3.73 (m, 1H), 3.63-3.51 (m, 3H), 2.97-2.68 (m, 2H), 1.14-0.96 (m, 4H). MS (ESI) m/z (M+H)⁺ 420.1.

To a mixture of compound 41E (0.54 g, 1.3 mmol) in DCM (50 mL) was added DMP (1.6 g, 3.9 mmol). The mixture was stirred at 25° C. for 50 min. The reaction was diluted with DCM (20 mL) and quenched by 40 mL of Sat. Na₂S₂O₃ solution and 40 mL of saturated NaHCO₃ solution and stirred for 5 min. After quenching the reaction, the reaction mixture was poured into separatory funnel and separated. The separated aqueous phase was extracted with DCM (30 mL×2). The combined organic phase was washed with brine (30 mL×2), dried over anhydrous Na₂SO₄, filtered and concentrated in vacuo to afford compound 41 (0.51 g, yield 93.6%) as pale yellow solid, which was used in next step without purification. ¹H NMR (DMSO-d₆, 400 MHz): δ. 8.61 (d, J=6.8 Hz, 1H), 8.11 (s, 1H), 7.59-7.48 (m, 2H), 7.36-7.19 (m, 8H), 5.11-4.96 (m, 1H), 3.87-3.78 (m, 1H), 3.75 (s, 3H), 3.24-3.13 (m, 1H), 2.97-2.84 (m, 1H), 1.12-0.98 (m, 4H). MS (ESI) m/z (M+H)⁺ 418.2.

To a mixture of compound 41 (0.15 g, 359.3 umol) in AcOH (2 mL) was added HCl (12M, 2 mL) in one portion. The mixture was stirred at 40° C. for 1 h. The mixture was diluted with H₂O (50 mL), and extracted with EA (30 mL×3). The combined organic phase was washed with brine (30 mL), dried over Na₂SO₄, filtered and concentrated in vacuo. The residue was purified by preparatory-HPLC (HCl condition) to afford compound 60 (40 mg, yield 27.6%) as pale yellow solid. ¹H NMR (DMSO-d₆, 400 MHz): δ. 8.52 (d, J=7.3 Hz, 1H), 8.11 (s, 1H), 7.60-7.50 (m, 2H), 7.36-7.18 (m, 8H), 5.08-4.97 (m, 1H), 3.88-3.74 (m, 1H), 3.24-3.12 (m, 1H), 2.95-2.81 (m, 1H), 1.14-0.96 (m, 4H). MS (ESI) m/z (M+H)⁺ 404.1.

### Methyl 2-oxo-4-phenyl-3-(4-phenyl-1,2,5-thiadiazole-3-carboxamido)butanoate (38) and 2-oxo-4-phenyl-3-(4-phenyl-1,2,5-thiadiazole-3-carboxamido)butanoic Acid (67)

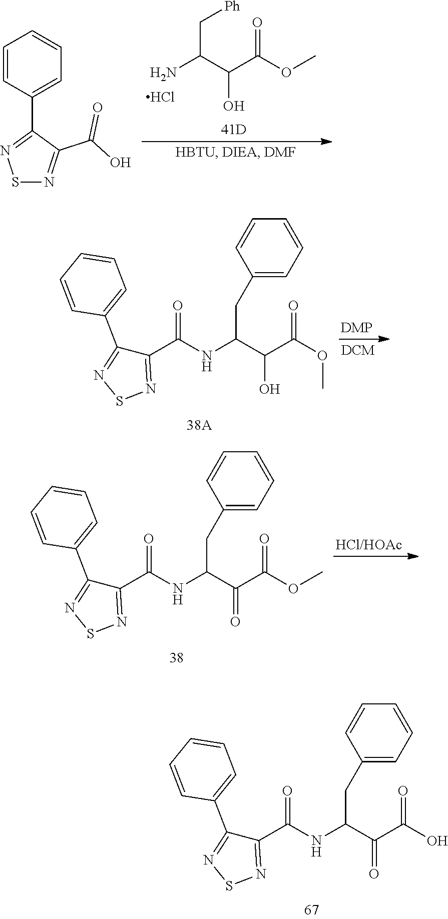 [SMILES: O=C(O)c1nsnc1-c1ccccc1 ; Cl ; COC(=O)C(O)C(N)Cc1ccccc1 ; COC(=O)C(O)C(Cc1ccccc1)NC(=O)c1nsnc1-c1ccccc1 ; COC(=O)C(=O)C(Cc1ccccc1)NC(=O)c1nsnc1-c1ccccc1 ; O=C(O)C(=O)C(Cc1ccccc1)NC(=O)c1nsnc1-c1ccccc1]

Compound 38 was prepared from 4-phenyl-1,2,5-thiadiazole-3-carboxylic acid and intermediate 41D using the same procedure as for compound 41. Compound 38 (0.440 g, yield 88.4%) was obtained as white solid, which was used in next step without purification. ¹H NMR (DMSO-d₆, 400 MHz) δ 9.27 (br d, J=6.0 Hz, 1H), 7.64 (br d, J=7.0 Hz, 2H), 7.51-7.38 (m, 3H), 7.31-7.21 (m, 5H), 5.32 (ddd, J=5.0, 7.5, 9.1 Hz, 1H), 3.81 (s, 3H), 3.28 (dd, J=4.9, 14.2 Hz, 1H), 3.03-2.98 (m, 1H). MS (ESI) m/z (M+H)⁺ 396.1.

Compound 67 was prepared from compound 38 using the same procedure as for compound 60. Compound 67 (0.123 g, yield 82.89%) was obtained as white solid. ¹H NMR (DMSO-d₆, 400 MHz): δ 7.84 (br d, J=6.5 Hz, 1H), 7.63-7.59 (m, 2H), 7.53-7.42 (m, 3H), 7.35-7.24 (m, 5H), 5.40 (ddd, J=4.8, 7.8, 9.0 Hz, 1H), 3.38 (dd, J=4.8, 14.1 Hz, 1H), 3.04 (dd, J=9.0, 14.1 Hz, 1H). MS (ESI) m/z (M+H)⁺ 382.1.

### Methyl 3-(3-(2-fluorophenyl)-1-methyl-1H-pyrazole-4-carboxamido)-2-oxo-phenylbutanoate (40) and 3-(3-(2-fluorophenyl)-1-methyl-1H-pyrazole-4-carboxamido)-2-oxo-4-phenylbutanoic Acid (65)

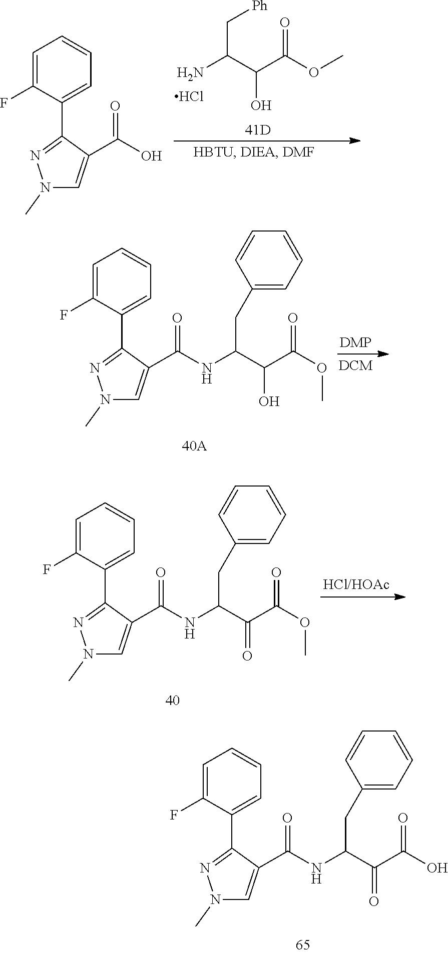 [SMILES: Cn1cc(C(=O)O)c(-c2ccccc2F)n1 ; Cl ; COC(=O)C(O)C(N)Cc1ccccc1 ; COC(=O)C(O)C(Cc1ccccc1)NC(=O)c1cn(C)nc1-c1ccccc1F ; COC(=O)C(=O)C(Cc1ccccc1)NC(=O)c1cn(C)nc1-c1ccccc1F ; Cn1cc(C(=O)NC(Cc2ccccc2)C(=O)C(=O)O)c(-c2ccccc2F)n1]

Compound 40 was prepared from 3-(2-fluorophenyl)-1-methyl-1H-pyrazole-4-carboxylic acid and intermediate 41D using the same procedure as for compound 41. Compound 40 (0.520 g, yield 87.1%) was obtained as yellow solid, which was used in next step without purification. ¹H NMR (DMSO-d₆, 400 MHz): δ 8.12 (br.s., 2H), 7.44-7.33 (m, 2H), 7.31-7.25 (m, 2H), 7.22-7.10 (m, 5H), 5.00 (br d, J=6.5 Hz, 1H), 3.91 (s, 3H), 3.75 (s, 3H), 3.17 (dd, J=5.3, 14.1 Hz, 1H), 2.94 (br.dd, J=8.9, 13.9 Hz, 1H). MS (ESI) m/z (M+H)⁺ 410.1.

Compound 65 was prepared from compound 40 using the same procedure as for compound 60. Compound 65 (60 mg, yield 40.5%) was obtained as white solid. ¹H NMR (DMSO-d₆, 400 MHz): δ 14.10 (s, 1H), 8.44 (d, J=7.0 Hz, 1H), 8.17 (s, 1H), 7.42-7.26 (m, 4H), 7.25-7.20 (m, 3H), 7.19-7.12 (m, 2H), 4.95 (ddd, J=4.8, 6.8, 9.5 Hz, 1H), 3.91 (s, 3H), 3.15 (dd, J=4.6, 13.9 Hz, 1H), 2.87 (dd, J=9.7, 13.9 Hz, 1H). MS (ESI) m/z (M+H)⁺ 396.2.

### Methyl 3-(4-(2-fluorophenyl)-2-methyloxazole-5-carboxamido)-2-oxo-4-phenylbutanoate (42) and 3-(4-(2-fluorophenyl)-2-methyloxazole-5-carboxamido)-2-oxo-4-phenylbutanoic Acid (64)

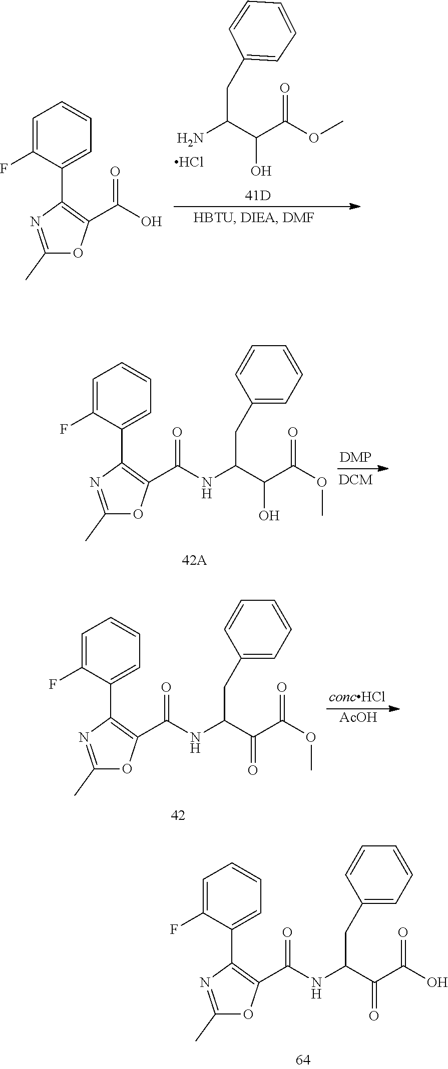

Compound 42 was prepared from 4-(2-fluorophenyl)-2-methyloxazole-5-carboxylic acid and intermediate 41D using the same procedure as for compound 41. Compound 42 (0.290 g, yield 67.0%) was obtained as light yellow solid, which was used in next step without purification. ¹H NMR (DMSO-d₆, 400 MHz): δ. 9.10 (d, J=7.1 Hz, 1H), 7.51-7.38 (m, 2H), 7.34-7.17 (m, 7H), 5.19-5.05 (m, 1H), 3.81-3.54 (m, 3H), 3.24-3.15 (m, 1H), 3.03-2.92 (m, 1H), 2.59-2.52 (m, 3H). MS (ESI) m/z (M+H)⁺ 411.1.

Compound 64 was prepared from compound 42 using the same procedure as for compound 60. Compound 64 (40 mg, yield 50.4%) was obtained as white solid. ¹H NMR (CD₃CN-d₃, 400 MHz): δ 7.54-7.39 (m, 2H), 7.37-7.11 (m, 8H), 5.31-5.16 (m, 1H), 3.29 (dd, J=5.0, 14.1 Hz, 1H), 3.00 (dd, J=8.8, 14.1 Hz, 1H), 2.50 (s, 3H). MS (ESI) m/z (M+H)⁺ 397.2.

### Methyl-3-(3-(3-fluorophenyl)-1-methyl-1H-pyrazole-4-carboxamido)-2-oxo-4-phenylbutanoate (74) and 3-(3-(3-fluorophenyl)-1-methyl-1H-pyrazole-4-carboxamido)-2-oxo-4-phenylbutanoic Acid (72)

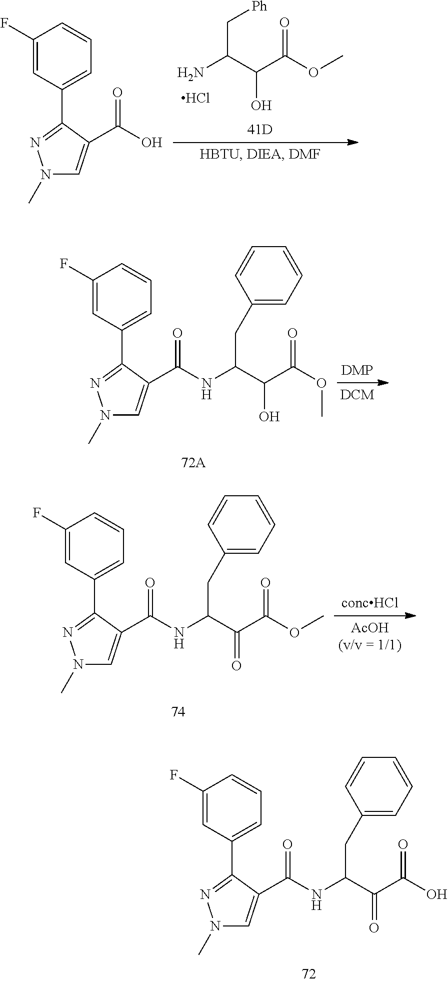 [SMILES: Cn1cc(C(=O)O)c(-c2cccc(F)c2)n1 ; Cl ; COC(=O)C(O)C(N)Cc1ccccc1 ; COC(=O)C(O)C(Cc1ccccc1)NC(=O)c1cn(C)nc1-c1cccc(F)c1 ; COC(=O)C(=O)C(Cc1ccccc1)NC(=O)c1cn(C)nc1-c1cccc(F)c1 ; Cn1cc(C(=O)NC(Cc2ccccc2)C(=O)C(=O)O)c(-c2cccc(F)c2)n1]

Compound 74 was prepared from 4-(2-fluorophenyl)-2-methyloxazole-5-carboxylic acid and intermediate 41D using the same procedure as for compound 41. Compound 74 (0.150 g, yield 75.3%) was obtained as light yellow solid, ¹H NMR (DMSO-d₆, 400 MHz): δ 8.73 (d, J=6.8 Hz, 1H), 8.06 (s, 1H), 7.45-7.29 (m, 4H), 7.28-7.20 (m, 4H), 7.14 (dt, J=2.1, 8.4 Hz, 1H), 5.06 (ddd, J=5.0, 6.8, 9.4 Hz, 1H), 3.91 (s, 3H), 3.76 (s, 3H), 3.20 (dd, J=4.9, 13.9 Hz, 1H), 2.91 (dd, J=9.5, 13.7 Hz, 1H). MS (ESI) m/z (M+H)⁺ 410.1.

Compound 72 was prepared from compound 74 using the same procedure as for compound 60. Compound 72 (50 mg, yield 64.7%) was obtained as white solid. ¹H NMR (DMSO-d₆, 400 MHz): δ 8.66 (br d, J=7.3 Hz, 1H), 8.07 (s, 1H), 7.44 (br d, J=8.0 Hz, 2H), 7.38-7.19 (m, 6H), 7.18-7.10 (m, 1H), 5.13-4.99 (m, 1H), 3.90 (s, 3H), 3.24-3.15 (m, 1H), 2.89 (dd, J=9.8, 14.1 Hz, 1H). MS (ESI) m/z (M+H)⁺ 396.2.

### Example 19—Compounds 58, 75, 76, 73, 78, 81, 84, 88, 90, 91, 92, 98, and 105

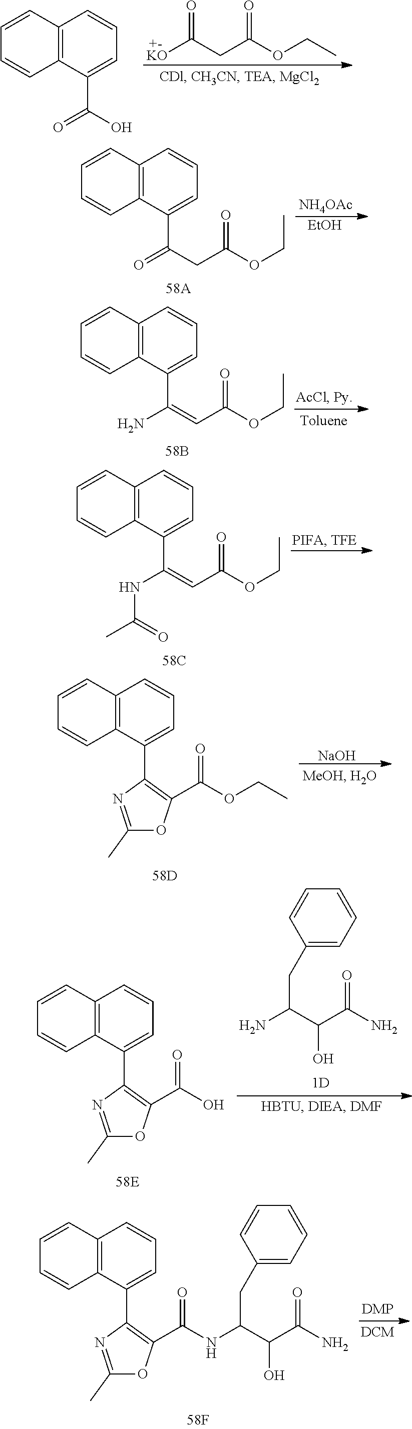

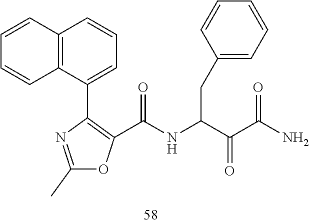 [SMILES: Cc1nc(-c2cccc3ccccc23)c(C(=O)NC(Cc2ccccc2)C(=O)C(N)=O)o1]

### N-(4-amino-3,4-dioxo-1-phenylbutan-2-yl)-2-methyl-4-(naphthalen-1-yl)oxazole-5-carboxamide (58)

(Flask A) A mixture of 1-naphthoic acid (25 g, 145.2 mmol) in CH₃CN (40 ml) was added CDI (28.3 g, 174.2 mmol), the mixture was stirred at 25° C. for 2 h. (Flask B) A mixture of ethyl potassium malonate (32.3 g, 191.7 mmol) in CH₃CN (200 mL) was added MgCl₂ (15.2, 64.0 mmol) and TEA (44.8 g, 435.6 mmol) in portions. The mixture was stirred 50° C. for 2 h. The solution in flask A was transferred to the slurry in flask B and the mixture was stirred at 70° C. for 12 h. The reaction mixture was quenched with HCl (3N, 600 mL) and the solution was concentrated under reduced pressure to remove solvent. The resulting concentrate extracted with MTBE (150 mL×3). The organic layer was washed with H₂O (150 mL×3), saturate NaHCO₃ (150 mL×3), and saturated NaCl (150 mL), dried over anhydrous Na₂SO₄, filtered and concentrated under reduced pressure to afford compound 58A (18 g, 46.9% yield) as colorless oil, which was used directly in next step. ¹H NMR (DMSO-d₆, 400 MHz) δ 8.59 (d, J=8.4 Hz, 1H), 8.19-8.15 (m, 2H), 8.03 (d, J=7.7 Hz, 1H), 7.68-7.58 (m, 3H), 4.31 (s, 2H), 4.09 (q, J=7.1 Hz, 2H), 1.11 (t, J=7.2 Hz, 3H). MS (ESI) m/z (M+H)⁺ 243.1.

To a mixture of compound 58A (18 g, 74.3 mmol, 1 eq) in EtOH (150 mL) was added NH₄OAc (45.8 g, 594.4 mmol) in one portion. The mixture was stirred at 90° C. for 24 h. The solvent was removed and concentrated under reduced pressure. EA (100 ml) and H₂O (50 mL) were added to the mixture, the organic layer was separated. The aqueous was extracted with EA (50 mL×2), the combined organic layer was washed with water (100 ml×2), saturate NaHCO₃ (100 mL×2), brine (100 mL×2). Then dried over anhydrous Na₂SO₄, filtered, concentrated under reduced pressure. The crude product was purified by column chromatography (SiO₂, Petroleum ether/Ethyl acetate=20/1 to 5/1) to afford compound 58B (16 g, 81.2% yield) as colorless oil. ¹H NMR (DMSO-d₆, 400 MHz) δ 8.21 (br. s, 1H), 8.13-8.06 (m, 1H), 8.02-7.95 (m, 2H), 7.61-7.42 (m, 5H), 4.51 (s, 1H), 4.08 (q, J=7.1 Hz, 2H), 1.21 (t, J=7.1 Hz, 3H). MS (ESI) m/z (M+H)⁺ 242.0.

Pyridine (10 mL, 124.3 mmol) was added to a stirred solution of compound 58B (3 g, 12.4 mmol) in toluene (20 mL) and the mixture reaction was cooled to 0° C. Acetyl chloride (6.7 mL, 93.3 mmol) was added dropwise, and the mixture was stirred for 6 h at 0° C. under an atmosphere of nitrogen. The compound 58B was monitored by LCMS, so additional acetyl chloride (20 mL, 279.8 mmol) was added into the reaction mixture and the mixture was stirred for 12 h at 0° C. under an atmosphere of nitrogen. The reaction was quenched with brine (30 ml), extracted with EA (50 ml×3) and dried over Na₂SO₄, and the solvent was evaporated in vacuo. The crude product was purified by column chromatography (SiO₂, PE/EA=20/1 to 5/1) to afford compound 58C (2.5 g, 66.4% yield) as white solid. ¹H NMR (DMSO-d₆, 400 MHz) δ 10.89 (s, 1H), 7.99-7.87 (m, 3H), 7.58-7.36 (m, 4H), 5.22-5.14 (m, 1H), 4.20 (q, J=7.1 Hz, 2H), 2.01 (s, 3H), 1.26 (t, J=7.2 Hz, 3H). MS (ESI) m/z (M+H)⁺ 284.1.

[Bis(trifluoroacetoxy)iodo]benzene (986.6 mg, 2.3 mmol) was added into a stirred solution of compound 58C (0.5 g, 1.8 mmol) in 2,2,2-trifluoroethanol (15 mL). The mixture was stirred for 30 min at 25° C. The reaction was quenched with saturated aqueous NaHCO₃ (20 ml) and the mixture diluted with EtOAc (20 ml) and extracted with EtOAc (20 ml×2). The organic layers were washed with water (15 ml×2), brine (15 ml), dried over Na₂SO₄, filtered and concentrated in vacuo The crude product was purified by column chromatography (SiO₂, Petroleum ether/Ethyl acetate=20/1 to 5/1) to afford compound 58D (380 mg, 74.2% yield) as pale yellow solid. ¹H NMR (DMSO-d₆, 400 MHz) δ 8.02 (dd, J=7.8, 14.6 Hz, 2H), 7.83 (d, J=8.3 Hz, 1H), 7.65-7.48 (m, 4H), 4.09 (q, J=7.0 Hz, 2H), 2.62 (s, 3H), 0.98 (t, J=7.0 Hz, 3H). MS (ESI) m/z (M+H)⁺ 282.0.

Compound 58D was hydrolyzed to yield intermediate 58E and this was reacted with intermediate 1D using the same procedure as described in Example 1 to yield compound 58. Compound 58 (0.140 g, yield 64.8%) was obtained as yellow solid, ¹H NMR (DMSO-d₆, 400 MHz) δ 8.63 (d, J=7.5 Hz, 1H), 8.06 (s, 1H), 7.97 (br d, J=7.8 Hz, 2H), 7.86-7.76 (m, 2H), 7.58-7.42 (m, 4H), 7.32-7.18 (m, 5H), 5.37-5.27 (m, 1H), 3.15 (br dd, J=3.4, 13.9 Hz, 1H), 2.94 (br dd, J=9.8, 13.8 Hz, 1H), 2.61 (s, 3H). MS (ESI) m/z (M+H)⁺ 428.1.

### N-(4-amino-3,4-dioxo-1-phenylbutan-2-yl)-4-(2-fluoro-3-methoxyphenyl)-2-methyloxazole-5-carboxamide (75)

Compound 75 was prepared from 2-fluoro-3-methoxybenzoic acid using the same procedures as described for compound 58 to yield the compound 75. Compound 75 (0.160 g, yield 53.6%) was obtained as yellow solid, ¹H NMR (DMSO-d₆, 400 MHz) δ 8.71 (d, J=7.6 Hz, 1H), 8.03 (s, 1H), 7.78 (s, 1H), 7.30-7.05 (m, 7H), 6.97-6.89 (m, 1H), 5.37-5.27 (m, 1H), 3.80 (s, 3H), 3.13 (dd, J=3.9, 13.9 Hz, 1H), 2.93 (dd, J=9.8, 14.2 Hz, 1H), 2.51 (s, 3H). MS (ESI) m/z (M+H)⁺ 426.1.

### N-(4-amino-3,4-dioxo-1-phenylbutan-2-yl)-4-(2,6-difluorophenyl)-2-methyloxazole-5-carboxamide (76)

Compound 76 was prepared from 2,6-difluorobenzoic acid using the same procedures as described for compound 58 to yield the compound 76. Compound 76 (0.153 g, yield 53.8%) was obtained as yellow solid, ¹H NMR (DMSO-d₆, 400 MHz) δ 8.88 (d, J=7.3 Hz, 1H), 8.07 (s, 1H), 7.82 (s, 1H), 7.58-7.46 (m, 1H), 7.35-7.07 (m, 7H), 5.39-5.28 (m, 1H), 3.16 (dd, J=3.5, 14.1 Hz, 1H), 2.96 (dd, J=10.0, 14.2 Hz, 1H), 2.57 (s, 3H). MS (ESI) m/z (M+H)⁺ 414.1.

### N-(4-amino-1-(4-fluorophenyl)-3,4-DIOXOBUTAN-2-yl)-4-(2-fluorophenyl)-2-methyloxazole-5-carboxamide (73)

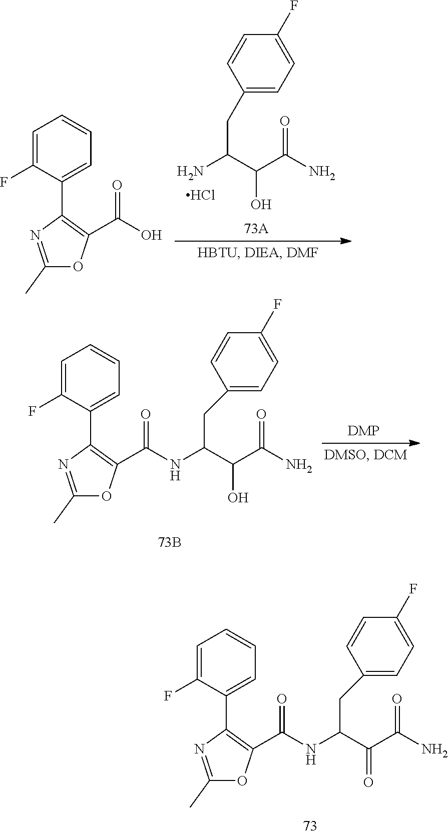 [SMILES: Cc1nc(-c2ccccc2F)c(C(=O)O)o1 ; Cl ; NC(=O)C(O)C(N)Cc1ccc(F)cc1 ; Cc1nc(-c2ccccc2F)c(C(=O)NC(Cc2ccc(F)cc2)C(O)C(N)=O)o1 ; Cc1nc(-c2ccccc2F)c(C(=O)NC(Cc2ccc(F)cc2)C(=O)C(N)=O)o1]

Compound 73 was prepared from 4-(2-fluorophenyl)-2-methyloxazole-5-carboxylic acid and intermediate 73A using the same procedures as described for Example 1 to yield the compound 73. Compound 73 (0.160 g, yield 73.08%) was obtained as white solid, ¹H NMR (DMSO-d₆, 400 MHz): δ 8.80 (d, J=7.3 Hz, 1H), 8.05 (s, 1H), 7.81 (s, 1H), 7.45 (q, J=7.3 Hz, 2H), 7.33-7.25 (m, 2H), 7.24-7.17 (m, 2H), 7.11 (t, J=8.8 Hz, 2H), 5.32 (s, 1H), 3.15 (dd, J=3.4, 13.9 Hz, 1H), 3.02-2.87 (m, 1H), 2.55 (s, 3H). MS (ESI) m/z (M+H)⁺ 414.1.

### N-(4-amino-3,4-dioxo-1-phenylbutan-2-yl)-4-(2,5-dimethylfuran-3-yl)-2-methyloxazole-5-carboxamide (78)

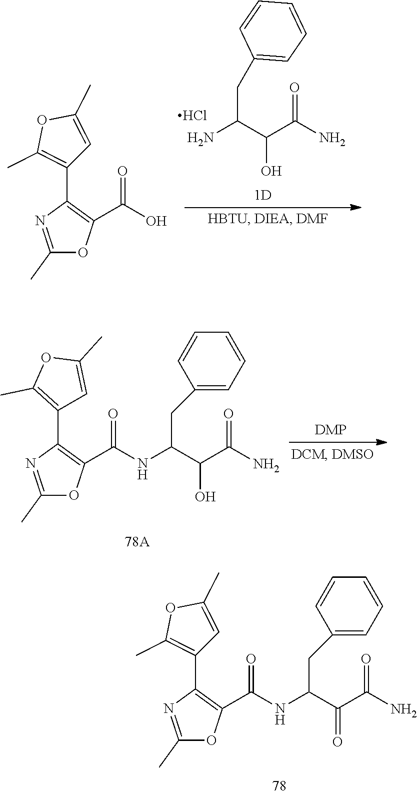 [SMILES: Cc1cc(-c2nc(C)oc2C(=O)O)c(C)o1 ; Cl ; NC(=O)C(O)C(N)Cc1ccccc1 ; Cc1cc(-c2nc(C)oc2C(=O)NC(Cc2ccccc2)C(O)C(N)=O)c(C)o1 ; Cc1cc(-c2nc(C)oc2C(=O)NC(Cc2ccccc2)C(=O)C(N)=O)c(C)o1]

Compound 78 was prepared from 4-(2,5-dimethylfuran-3-yl)-2-methyloxazole-5-carboxylic acid and intermediate 1D using the same procedures as described for compound 58 to yield the compound 78. Compound 78 (65 mg, yield 40.9%) was obtained as white solid, ¹H NMR (400 MHz, DMSO-d₆) δ 8.60 (d, J=7.3 Hz, 1H), 8.14-8.04 (m, 1H), 7.81 (s, 1H), 7.29-7.23 (m, 4H), 7.20-7.15 (m, 1H), 6.57 (s, 1H), 5.39-5.34 (m, 1H), 3.16 (dd, J=3.8, 13.8 Hz, 1H), 2.95 (dd, J=9.8, 13.9 Hz, 1H), 2.46 (s, 3H), 2.36 (s, 3H), 2.19-2.12 (m, 3H). MS (ESI) m/z (M+H)⁺ 396.1.

### N-(4-amino-3,4-dioxo-1-phenylbutan-2-yl)-4-(2,5-dichlorofuran-3-yl)-2-methyloxazole-5-carboxamide (81)

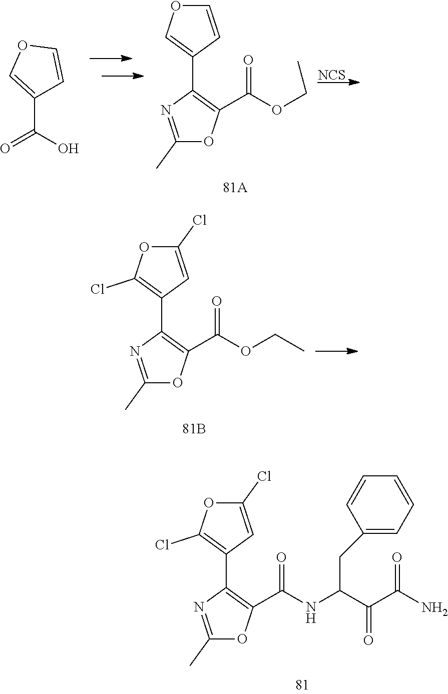

Compound 81A was prepared from furan-3-carboxylic acid using the same procedures as described for compound 58D to yield the compound 81A. Compound 81A (1.28 g, yield 64.2%) was obtained as white solid, ¹H NMR (400 MHz, CDCl₃) δ 8.37 (s, 1H), 7.48 (s, 1H), 7.13-7.07 (m, 1H), 4.43 (q, J=7.3 Hz, 2H), 2.55 (s, 3H), 1.43 (t, J=7.1 Hz, 3H). MS (ESI) m/z (M+H)⁺ 221.9.

To a solution of compound 81A (300 mg, 1.36 mmol) in DMF (3 mL) was added NCS (580 mg, 4.34 mmol). The mixture was stirred at 100° C. for 6 hr. The reaction was diluted with H₂O (30 mL), extracted with EA (20 mL×3), the organic phase was dried over Na₂SO₄, filtered, and concentrated to give a residue. The residue was purified by flash silica gel chromatography (PE:EA=10:1 to 5:1). Compound 81B (80 mg, yield: 20.3%) was obtained as a white solid. ¹H NMR (400 MHz, CDCl₃) δ 6.85 (s, 1H), 4.40 (q, J=7.2 Hz, 2H), 2.58 (s, 3H), 1.39 (br t, J=7.1 Hz, 3H).

Compound 81B was hydrolyzed to yield the intermediate acid which was reacted with intermediate 1D using the same procedure as described in Example 1 to yield compound 81. Compound 81 (68 mg, yield 88.3%) was obtained as pale yellow solid, ¹H NMR (400 MHz, DMSO-d₆) δ 8.93 (d, J=7.6 Hz, 1H), 8.10 (s, 1H), 7.83 (s, 1H), 7.27-7.24 (m, 4H), 7.19-7.15 (m, 1H), 7.00 (s, 1H), 5.42-5.31 (m, 1H), 3.22-3.13 (m, 1H), 2.96-2.89 (m, 1H), 2.50 (s, 3H). MS (ESI) m/z (M+H)⁺ 436.0.

### N-(4-amino-3,4-dioxo-1-phenylbutan-2-yl)-2-methyl-4-(2-methylfuran-3-yl)oxazole-5-carboxamide (84)

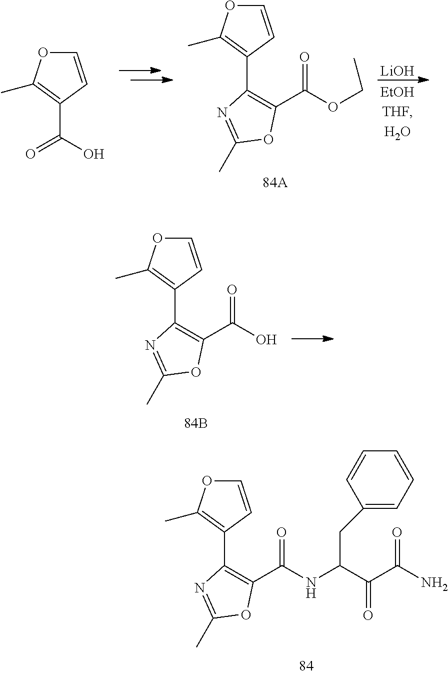

Compound 84 was prepared from 2-methylfuran-3-carboxylic acid via intermediates 84A and 84B using the same procedures as described for compound 58 to yield the compound 84. Compound 84 (60 mg, yield 37.52%) was obtained as white solid. MS (ESI) m/z (M+1)⁺ 382.1. ¹H NMR (DMSO-d₆, 400 MHz): δ 8.65 (d, J=7.2 Hz, 1H), 8.08 (br. s, 1H), 7.81 (br. s, 1H), 7.45 (d, J=2.0 Hz, 1H), 7.30-7.21 (m, 4H), 7.21-7.13 (m, 1H), 6.94 (d, J=2.0 Hz, 1H), 5.45-5.32 (m, 1H), 3.21-3.09 (m, 1H), 3.01-2.88 (m, 1H), 2.48 (s, 3H), 2.39 (s, 3H).

### N-(4-amino-3,4-dioxo-1-phenylbutan-2-yl)-4-(benzo[b]thiophen-4-yl)-2-methyloxazole-5-carboxamide (88)

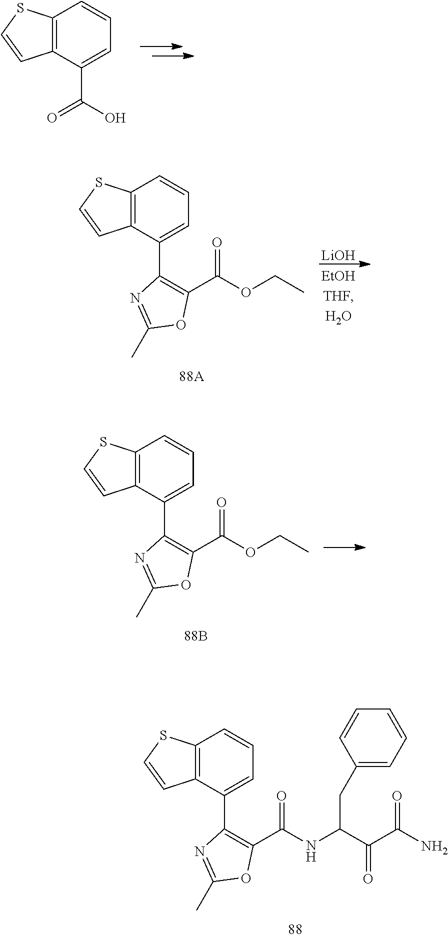

Compound 88 was prepared from benzo[b]thiophene-4-carboxylic acid via intermediates 88A and 88B using the same procedures as described for compound 58 to yield the compound 88. Compound 88 (110 mg, yield 92.6%) was obtained as yellow solid. ¹H NMR (400 MHz, DMSO-d₆) δ 8.73 (d, J=7.3 Hz, 1H), 8.07-7.98 (m, 2H), 7.80 (s, 1H), 7.71 (d, J=5.6 Hz, 1H), 7.52-7.47 (m, 1H), 7.37 (d, J=5.4 Hz, 1H), 7.32 (d, J=7.8 Hz, 1H), 7.30-7.16 (m, 5H), 5.38-5.28 (m, 1H), 3.14 (dd, J=3.5, 13.8 Hz, 1H), 2.92 (dd, J=9.9, 14.1 Hz, 1H), 2.56 (s, 3H). MS (ESI) m/z (M+H)⁺=434.1.

### N-(4-amino-3,4-dioxo-1-phenylbutan-2-yl)-4-(2-chlorofuran-3-yl)-2-methyloxazole-5-carboxamide (90)

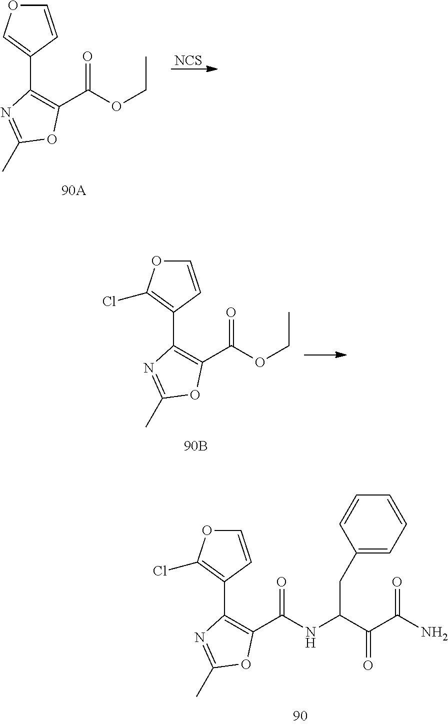

To a solution of compound 90A (400 mg, 1.81 mmol) in DMF (3 mL) was added NCS (266 mg, 1.99 mmol). The mixture was stirred at 15° C. for 16 h. Then the mixture was stirred at 25° C. for 16 h. The reaction was diluted with H₂O (40 mL), extracted with EA (30 mL×2), the organic phase was dried over Na₂SO₄, filtered, and concentrated to give a residue. The residue was purified by flash silica gel chromatography (PE:EA=10:1 to 4:1). Compound 90B (300 mg, yield: 64.9%) was obtained as a white solid. ¹H NMR (400 MHz, CDCl₃) δ 7.37 (d, J=2.0 Hz, 1H), 6.98 (d, J=2.2 Hz, 1H), 4.46-4.34 (m, 2H), 2.62-2.53 (m, 3H), 1.46-1.33 (m, 3H).

Compound 90B was hydrolyzed to yield the intermediate acid which was reacted with intermediate 1D using the same procedure as described in Example 1 to yield compound 90. Compound 90 (90 mg, yield 51.8%) was obtained as white solid, ¹H NMR (400 MHz, DMSO-d₆) δ 8.86 (d, J=7.6 Hz, 1H), 8.12 (s, 1H), 7.85 (s, 1H), 7.73-7.68 (m, 1H), 7.32-7.26 (m, 4H), 7.25-7.17 (m, 1H), 7.07-6.99 (m, 1H), 5.43-5.38 (m, 1H), 3.19 (dd, J=3.8, 14.1 Hz, 1H), 2.97 (dd, J=10.0, 13.9 Hz, 1H), 2.53 (s, 3H). MS (ESI) m/z (M+H)⁺ 402.1.

### N-(4-amino-3,4-dioxo-1-phenylbutan-2-yl)-4-(benzo[b]thiophen-7-yl)-2-methyloxazole-5-carboxamide (91)

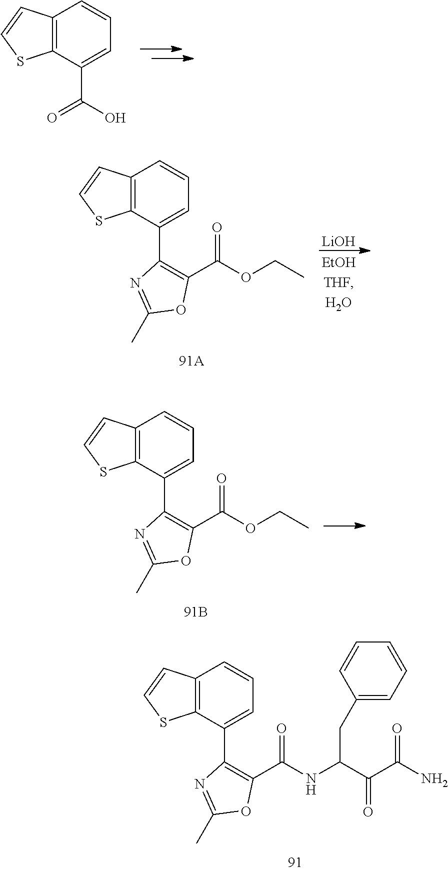

Compound 91 was prepared from benzo[b]thiophene-7-carboxylic acid via intermediates 91A and 91B using the same procedures as described for compound 58 to yield the compound 91. Compound 91 (15 mg, yield 49.6%) was obtained as white solid. ¹H NMR (400 MHz, DMSO-d₆) δ 8.90 (d, J=7.5 Hz, 1H), 8.12 (s, 1H), 8.02 (d, J=7.3 Hz, 1H), 7.93-7.85 (m, 2H), 7.75 (d, J=5.8 Hz, 1H), 7.48 (d, J=5.8 Hz, 1H), 7.39 (d, J=7.8 Hz, 1H), 7.31-7.28 (m, 3H), 7.25-7.16 (m, 2H), 5.45-5.41 (m, 1H), 3.20 (dd, J=3.9, 13.9 Hz, 1H), 2.98 (dd, J=9.8, 13.8 Hz, 1H), 2.62 (s, 3H). MS (ESI) m/z (M+H)⁺ 434.1.

### N-(4-amino-3,4-dioxo-1-phenylbutan-2-yl)-4-(5-chlorofuran-3-yl)-2-methyloxazole-5-carboxamide (92)

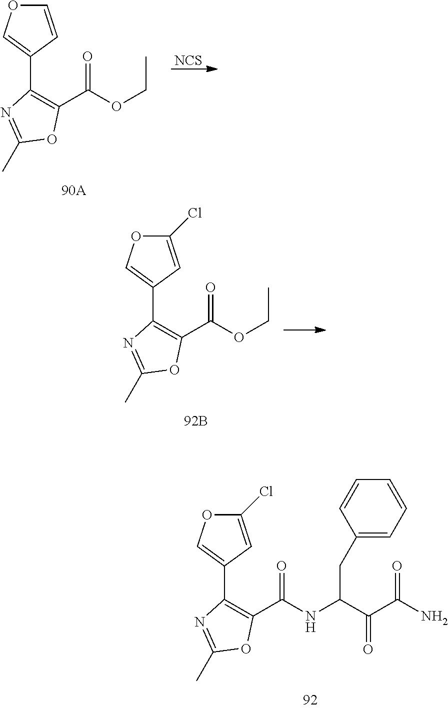

To a solution of compound 90A (400 mg, 1.81 mmol) in DMF (3 mL) was added NCS (266 mg, 1.99 mmol). The mixture was stirred at 15° C. for 16 h. Then the mixture was stirred at 25° C. for 16 h. The reaction was diluted with H₂O (40 mL), extracted with EA (30 mL×2), the organic phase was dried over Na₂SO₄, filtered, and concentrated to give a residue. The residue was purified by flash silica gel chromatography (PE:EA=10:1 to 4:1). Compound 92B (55 mg, yield: 11.9%) was obtained as a white solid. ¹H NMR (400 MHz, CDCl₃) δ 8.22 (s, 1H), 6.93 (d, J=1.0 Hz, 1H), 4.46-4.40 (m, 2H), 2.54 (s, 3H), 1.42 (t, J=7.1 Hz, 3H).

Compound 92B was hydrolyzed to yield the intermediate acid which was reacted with intermediate 1D using the same procedure as described in Example 1 to yield compound 92. Compound 92 (45 mg, yield 61.3%) was obtained as white solid, ¹H NMR (400 MHz, DMSO-d₆) δ 8.90 (d, J=7.6 Hz, 1H), 8.33 (d, J=1.0 Hz, 1H), 8.15 (s, 1H), 7.87 (s, 1H), 7.34-7.27 (m, 4H), 7.24-7.16 (m, 1H), 7.02 (d, J=1.0 Hz, 1H), 5.45-5.41 (m, 1H), 3.21 (dd, J=3.9, 13.9 Hz, 1H), 3.00 (dd, J=9.9, 14.1 Hz, 1H), 2.53 (s, 3H). MS (ESI) m/z (M+H)⁺ 402.1.

### N-(4-amino-3,4-dioxo-1-phenylbutan-2-yl)-4-(5-chloro-2-methylfuran-3-yl)-2-methyloxazole-5-carboxamide (98)

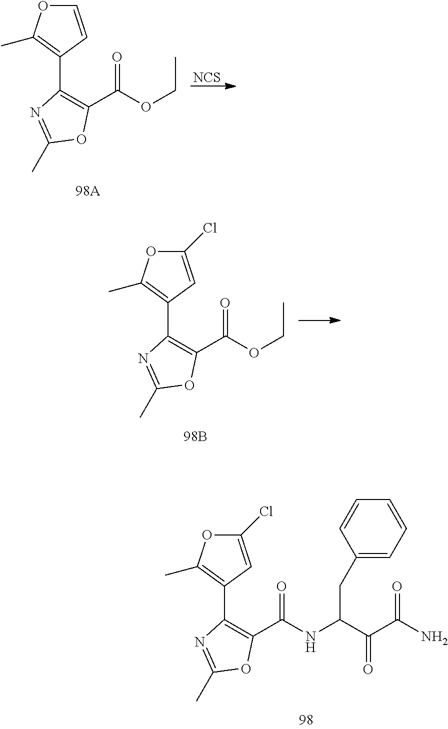

To a solution of compound 98A (100 mg, 0.42 mmol) in DMF (5 mL) was added NCS (57 mg, 0.42 mmol). The mixture was stirred at 20° C. for 12 h. The mixture was washed with H₂O (20 mL), extracted with EtOAc (15 mL×2). The organics were collected and concentrated. The residue was purified by column (PE:EA=10:1) to give compound 2 (60 mg, yield: 52.34%) as colorless solid. ¹H NMR (DMSO-d₆, 400 MHz): δ 6.94 (s, 1 h), 4.34-4.27 (m, 2H), 2.53 (s, 3H), 2.50 (s, 3H), 1.31-1.27 (m, 3H).

Compound 98B was hydrolyzed to yield the intermediate acid which was reacted with intermediate 1D using the same procedure as described in Example 1 to yield compound 98. Compound 98 (80 mg, yield 40.15%) was obtained as light yellow solid, MS (ESI) m/z (M+1)⁺ 416.1. ¹H NMR (DMSO-d₆, 400 MHz): δ 8.77 (d, J=7.6 Hz, 1H), 8.09 (br. s, 1H), 7.82 (br. s, 1H), 7.29-7.22 (m, 4H), 7.20-7.13 (m, 1H), 6.89 (s, 1H), 5.41-5.32 (m, 1H), 3.20-3.12 (m, 1H), 3.00-2.89 (m, 1H), 2.49 (s, 3H), 2.41 (s, 3H).

### 2-(5-(ethoxycarbonyl)-2-methyloxazol-4-yl)-N,N,N-trimethylbenzenaminium (105)

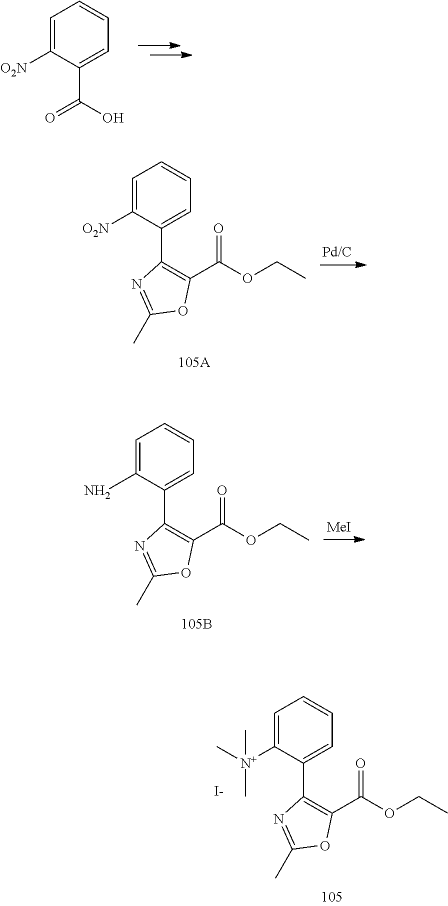

2-nitrobenzoic acid was subjected to conditions as described for compound 58 to yield the compound 105A. Compound 105A (480 mg, 60.4% yield) was obtained as a yellow oil. ¹H NMR (400 MHz, DMSO-d₆) δ 8.14-8.09 (m, 1H), 7.87-7.80 (m, 1H), 7.78-7.70 (m, 2H), 4.21-4.13 (m, 2H), 2.58 (s, 3H), 1.17-1.11 (m, 3H).

To a solution of compound 105A (200 mg, 724.00 umol) in EtOH (20 mL) was added Pd/C (45 mg, 72.40 umol, 10% purity) and NH₃.H₂O (2.17 mmol, 270 uL, 30% purity). The mixture was stirred at 25° C. for 1 hr under H₂ balloon (15 psi). The mixture was filtered and concentrated. The residue was purified by column chromatography (SiO₂, Petroleum ether/Ethyl acetate=2/1 to Oil). Compound 105B (75 mg, 42.1% yield) was obtained as a yellow solid. ¹H NMR (400 MHz, CDCl₃) δ 7.58-7.48 (m, 1H), 7.22-7.16 (m, 1H), 6.79-6.72 (m, 2H), 4.67 (br s, 2H), 4.37-4.30 (m, 2H), 2.58 (s, 3H), 1.34-1.28 (m, 3H).

To a solution of Compound 105B (120 mg, 487.29 umol) and MeI (2.77 g, 19.49 mmol, 1.21 mL) in acetone (3 mL) was added K₂CO₃ (300 mg, 2.17 mmol). The mixture was stirred at 40° C. for 48 h, and added MeI (2.77 g, 19.49 mmol, 1.21 mL). The mixture was stirred at 40° C. for 48 h, The reaction was filtered, the filtrate was concentrated, The residue was purified by preparatory-TLC (SiO₂, DCM:EA=1:1). Compound 105 (40 mg, 27.2% yield) was obtained as a yellow solid. ¹H NMR (400 MHz, DMSO-d₆) δ 8.10 (d, J=8.3 Hz, 1H), 7.76 (t, J=7.5 Hz, 1H), 7.64 (t, J=7.4 Hz, 1H), 7.48 (d, J=7.3 Hz, 1H), 4.13 (q, J=7.2 Hz, 2H), 3.74-3.47 (m, 9H), 2.61 (s, 3H), 1.06 (t, J=7.0 Hz, 3H). MS (ESI) m/z (M+H)⁺ 433.1.

### Example 20—Compounds 80, 83, 87, 89, 95, 96, and 97

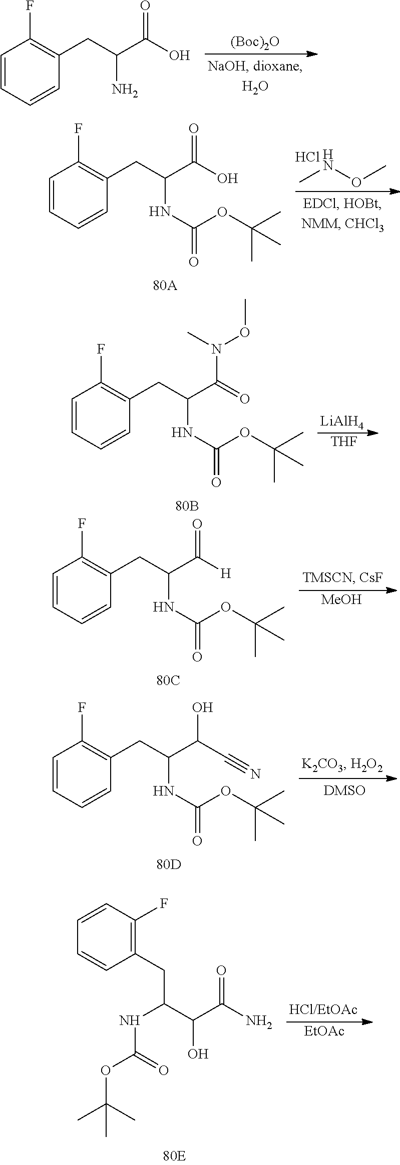

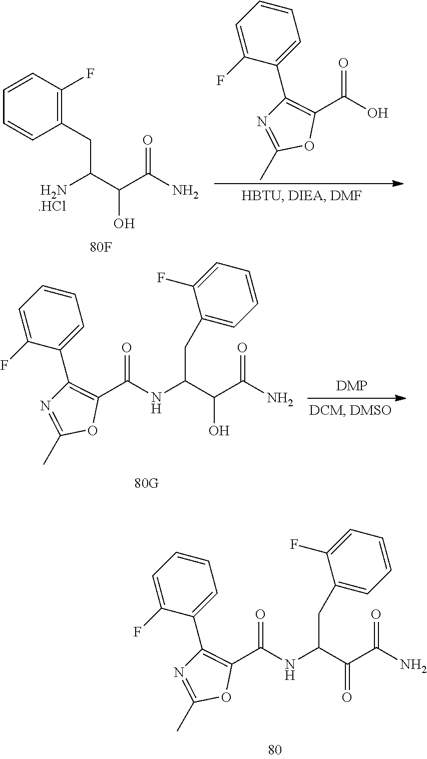 [SMILES: NC(=O)C(O)C(N)Cc1ccccc1F ; ".HCl" ; Cc1nc(-c2ccccc2F)c(C(=O)O)o1 ; Cc1nc(-c2ccccc2F)c(C(=O)NC(Cc2ccccc2F)C(O)C(N)=O)o1 ; Cc1nc(-c2ccccc2F)c(C(=O)NC(Cc2ccccc2F)C(=O)C(N)=O)o1]

### N-(4-amino-1-(2-fluorophenyl)-3,4-dioxobutan-2-yl)-4-(2-fluorophenyl)-2-methyloxazole-5-carboxamide (80)

To a solution of 2-amino-3-(2-fluorophenyl)propanoic acid (5.77 g, 31.50 mmol) in dioxane (45 mL) was added NaOH (1.95 g, 48.82 mmol) in H₂O (12 mL) and Boc₂O (8.66 g, 39.69 mmol, 9.12 mL) in dioxane (15 mL). The mixture was stirred at 25° C. for 20 h. The reaction was concentrated under reduced pressure and H₂O (60 mL) was added to the mixture. The aqueous was treated with HCl (0.5M) until pH ˜3 and the reaction was extracted with EA (50 mL×3). The combined organic layer was washed with H₂O (50 mL) and brine (50 mL), dried over anhydrous Na₂SO₄, filtered, concentrated to give a residue. Compound 80A (8.58 g, yield: 96.2%) was obtained as a yellow solid which was used into the next step without further purification. ¹H NMR (400 MHz, DMSO-d₆) δ 12.66 (br s, 1H), 7.35-7.22 (m, 2H), 7.17-7.07 (m, 3H), 4.19-4.07 (m, 1H), 3.13 (br dd, J=4.9, 13.9 Hz, 1H), 2.81 (br dd, J=10.5, 13.7 Hz, 1H), 1.30 (s, 9H).

To a mixture of compound 80A (8.58 g, 30.29 mmol) and N-methoxymethanamine (4.14 g, 42.41 mmol, HCl), HOBt (4.50 g, 33.32 mmol) in CHCl₃ (100 mL) was added NMM (12.25 g, 121.16 mmol, 13.32 mL) dropwise. Then EDCI (8.13 g, 42.41 mmol) was added to the mixture and the mixture was stirred at 25° C. for 18 h. The reaction was concentrated under reduced pressure. H₂O (100 mL) and EA (100 mL) were added to the mixture, the organic layer was separated. The aqueous layer was extracted with EA (60 mL×2). The combined organic layer was washed with HCl (0.5M, 100 mL), saturated NaHCO₃ (100 mL), dried over anhydrous Na₂SO₄, filtered, concentrated under reduced pressure to give a residue. Compound 80B (9.26 g, yield: 91.7%) was obtained as a yellow solid, which was used into the next step without further purification. ¹H NMR (400 MHz, DMSO-d₆) δ 7.38-7.19 (m, 2H), 7.17-6.99 (m, 3H), 4.66 (br s, 1H), 3.67 (br s, 3H), 3.13-3.02 (m, 3H), 2.95 (br dd, J=4.5, 13.6 Hz, 1H), 2.76-2.61 (m, 1H), 1.27 (s, 9H). MS (ESI) m/z (M+Na)⁺348.9.

To a solution of LiAlH₄ (1.18 g, 31.21 mmol) in THF (50 mL) was added dropwise a solution of compound 80B (9.26 g, 28.37 mmol) in THF (100 mL) at 0° C. under N₂ atmosphere. After addition, the mixture was stirred at 0° C. for 2 h. The reaction mixture was added EA (100 mL) and HCl (1M, 100 mL) at 0° C. The organic layer was separated and the aqueous layer was extracted with EA (100 mL×2). The combined organic layer was washed with HCl (1M, 100 mL), H₂O (100 mL), brine (100 mL). The combined organic layer was dried over anhydrous Na₂SO₄, filtered, concentrated under reduced pressure to give a residue. Compound 80C (5.65 g, yield: 74.5%) was obtained as a yellow oil, which was used into the next step without further purification. ¹H NMR (400 MHz, DMSO-d₆) δ 9.50 (s, 1H), 7.37 (br d, J=7.3 Hz, 1H), 7.31-7.22 (m, 2H), 7.16-7.08 (m, 2H), 4.03 (q, J=6.8 Hz, 1H), 3.13 (br dd, J=4.6, 13.9 Hz, 1H), 2.74 (br dd, J=10.1, 13.6 Hz, 1H), 1.32 (s, 9H).

To a solution of compound 80C (2 g, 7.48 mmol) and CsF (568 mg, 3.74 mmol) in MeOH (50 mL) was added dropwised trimethylsilylformonitrile (890.76 mg, 8.98 mmol, 1.12 mL) at 0° C. The mixture was warmed to 20° C. and stirred for 5 h. The reaction mixture was concentrated, then diluted with H₂O (30 mL), extracted with EA (30 mL×3), the combined organic layers were dried over Na₂SO₄, filtered and concentrated to give a residue. Compound 80D (2.62 g, crude) was obtained as a yellow oil, which was used into the next step without further purification. ¹H NMR (400 MHz, DMSO-d₆) δ 7.23 (br d, J=7.6 Hz, 2H), 7.15-7.03 (m, 3H), 4.63-4.28 (m, 1H), 3.93-3.75 (m, 1H), 3.12-2.93 (m, 1H), 2.78-2.58 (m, 1H), 1.25 (s, 4.5H), 1.22 (s, 4.5H).

To a solution of compound 80D (530 mg, 1.80 mmol) in DMSO (10 mL) was added K₂CO₃ (498 mg, 3.60 mmol) and H₂O₂ (3.06 g, 27.01 mmol, 2.60 mL, 30% purity) was added dropwise to the mixture. The mixture was stirred at 20° C. for 3 h. The reaction was quenched with saturated Na₂S₂O₃ (20 mL) and diluted with H₂O (30 mL). The mixture was extracted with EA (40 mL×3) and the combined organic layer was washed with H₂O (40 mL), brine (40 mL), dried over anhydrous Na₂SO₄, filtered, concentrated to give a residue. Compound 80E (507 mg, yield: 90.1%) was obtained as a pale yellow solid, which was used into the next step without further purification. ¹H NMR (400 MHz, DMSO-d₆) δ 7.34-7.16 (m, 4H), 7.14-7.04 (m, 2H), 6.52-6.04 (m, 1H), 5.69 (dd, J=6.0, 12.6 Hz, 1H), 4.04 (br d, J=8.8 Hz, 1H), 3.94-3.74 (m, 1H), 2.90-2.61 (m, 2H), 1.24 (s, 9H).

To a solution of compound 80E (1.39 g, 4.45 mmol) in EtOAc (15 mL) was added HCl/EtOAc (4M, 15 mL). The mixture was stirred at 25° C. for 2 h. The precipitate was filtered and filtered cake was washed with EA (20 mL). The solid was dried under reduced pressure. Compound 80F (933 mg, yield: 84.3%, HCl) was obtained as a pale yellow solid. ¹H NMR (400 MHz, DMSO-d₆) δ 8.17-7.90 (m, 3H), 7.55-7.43 (m, 2H), 7.43-7.23 (m, 2H), 7.21-7.07 (m, 2H), 6.74-6.36 (m, 1H), 4.23-3.77 (m, 1H), 3.72-3.53 (m, 1H), 2.92 (br d, J=7.1 Hz, 1H), 2.82 (br d, J=7.1 Hz, 1H).

Compound 80F and 4-(2-fluorophenyl)-2-methyloxazole-5-carboxylic acid were coupled using the same conditions as for intermediates 58E and 1D and then used procedures as described in Example 1 to yield compound 80. Compound 80 (95 mg, yield 60.7%) was obtained as white solid, ¹H NMR (400 MHz, DMSO-d₆) δ 8.77 (d, J=7.3 Hz, 1H), 8.01 (s, 1H), 7.75 (s, 1H), 7.50-7.38 (m, 2H), 7.32-7.17 (m, 4H), 7.16-7.06 (m, 2H), 5.39-5.29 (m, 1H), 3.22 (br dd, J=4.8, 14.3 Hz, 1H), 3.01 (dd, J=9.0, 13.9 Hz, 1H), 2.53 (s, 3H). MS (ESI) m/z (M+H)⁺ 414.1.

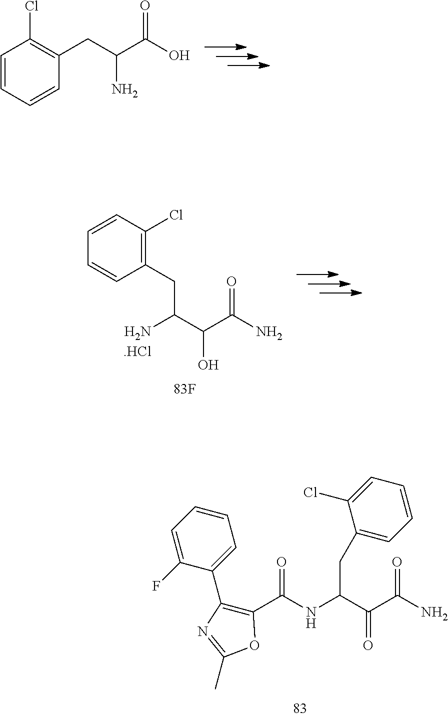 [SMILES: NC(Cc1ccccc1Cl)C(=O)O ; NC(=O)C(O)C(N)Cc1ccccc1Cl ; ".HCl" ; Cc1nc(-c2ccccc2F)c(C(=O)NC(Cc2ccccc2Cl)C(=O)C(N)=O)o1]

### N-(4-amino-1-(2-chlorophenyl)-3,4-dioxobutan-2-yl)-4-(2-fluorophenyl)-2-methyloxazole-5-carboxamide (83)

Compound 2-amino-3-(2-chlorophenyl)propanoic acid was converted to intermediate 83F which was then coupled with 4-(2-fluorophenyl)-2-methyloxazole-5-carboxylic acid using the same conditions as for compound 80 and then further used procedures as described in Example 1 to yield compound 83. Compound 83 (120 mg, yield 36%) was obtained as white solid, ¹H NMR (DMSO-d₆, 400 MHz): δ 8.89 (d, J=7.6 Hz, 1H), 8.03 (s, 1H), 7.75 (s, 1H), 7.50-7.39 (m, 3H), 7.38-7.30 (m, 1H), 7.29-7.17 (m, 4H), 5.46-5.33 (m, 1H), 3.33-3.26 (m, 1H), 3.08 (dd, J=9.8, 14.2 Hz, 1H), 2.54 (s, 3H). MS (ESI) m/z (M+H)⁺ 430.1.

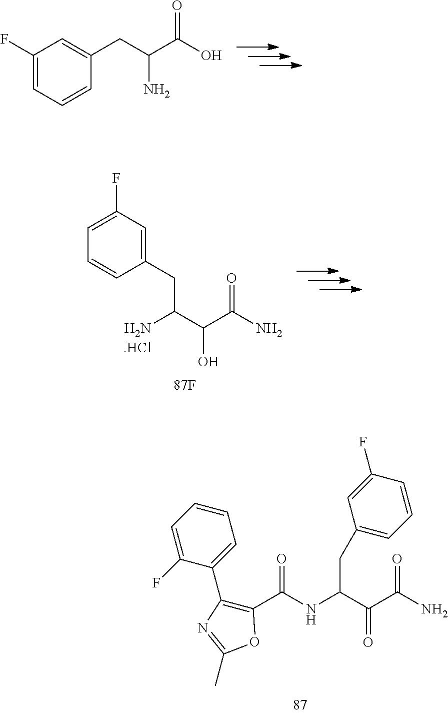 [SMILES: NC(Cc1cccc(F)c1)C(=O)O ; NC(=O)C(O)C(N)Cc1cccc(F)c1 ; ".HCl" ; Cc1nc(-c2ccccc2F)c(C(=O)NC(Cc2cccc(F)c2)C(=O)C(N)=O)o1]

### N-(4-amino-1-(3-fluorophenyl)-3,4-dioxobutan-2-yl)-4-(2-fluorophenyl)-2-methyloxazole-5-carboxamide (87)

Compound 2-amino-3-(3-fluorophenyl)propanoic acid was converted to intermediate 87F which was then coupled with 4-(2-fluorophenyl)-2-methyloxazole-5-carboxylic acid using the same conditions as for compound 80 and then further used procedures as described in Example 1 to yield compound 87. Compound 87 (160 mg, yield 55%) was obtained as light yellow solid, ¹H NMR (DMSO-d₆, 400 MHz): δ 8.87 (d, J=7.5 Hz, 1H), 8.07 (s, 1H), 7.83 (s, 1H), 7.50-7.40 (m, 2H), 7.38-7.30 (m, 1H), 7.25-7.17 (m, 2H), 7.13-7.01 (m, 3H), 5.42-5.27 (m, 1H), 3.19 (dd, J=3.8, 14.1 Hz, 1H), 2.98 (dd, J=9.9, 13.9 Hz, 1H), 2.55 (s, 3H). MS (ESI) m/z (M+H)⁺ 414.1.

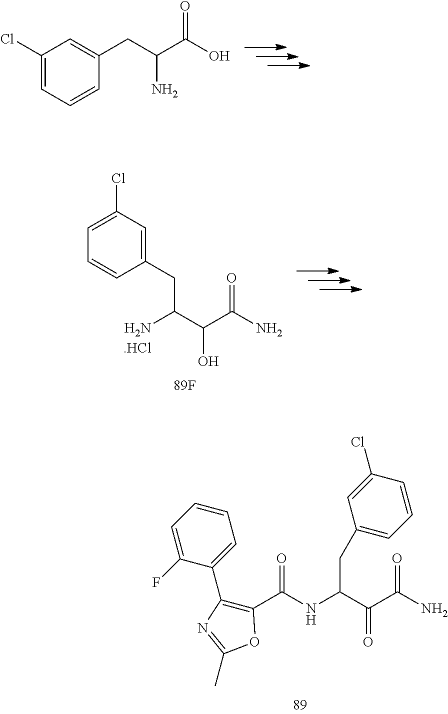 [SMILES: NC(Cc1cccc(Cl)c1)C(=O)O ; NC(=O)C(O)C(N)Cc1cccc(Cl)c1 ; ".HCl" ; Cc1nc(-c2ccccc2F)c(C(=O)NC(Cc2cccc(Cl)c2)C(=O)C(N)=O)o1]

### N-(4-amino-1-(3-chlorophenyl)-3,4-dioxobutan-2-yl)-4-(2-fluorophenyl)-2-methyloxazole-5-carboxamide (89)

Compound 2-amino-3-(3-chlorophenyl)propanoic acid was converted to intermediate 89F which was then coupled with 4-(2-fluorophenyl)-2-methyloxazole-5-carboxylic acid using the same conditions as for compound 80 and then further used procedures as described in Example 1 to yield compound 89. Compound 89 (70 mg, yield 31.5%) was obtained as white solid. ¹H NMR (400 MHz, DMSO-d₆) δ 8.89 (d, J=7.6 Hz, 1H), 8.09 (s, 1H), 7.84 (s, 1H), 7.44 (q, J=7.3 Hz, 2H), 7.35-7.25 (m, 3H), 7.25-7.16 (m, 3H), 5.37-5.26 (m, 1H), 3.17 (dd, J=3.7, 13.9 Hz, 1H), 2.95 (dd, J=10.0, 13.9 Hz, 1H), 2.55 (s, 3H). MS (ESI) m/z (M+H)⁺ 430.1.

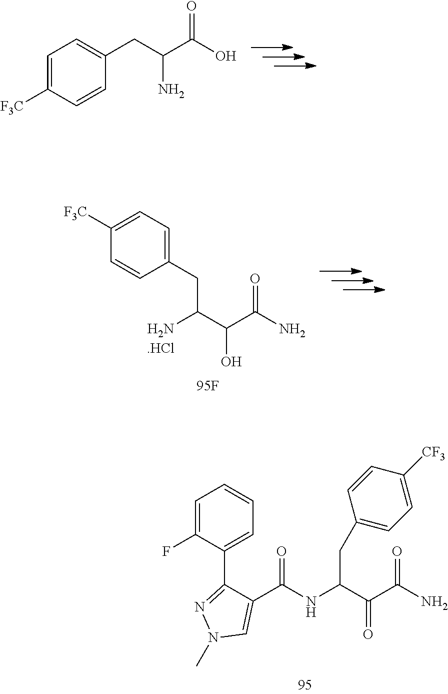 [SMILES: NC(Cc1ccc(C(F)(F)F)cc1)C(=O)O ; NC(=O)C(O)C(N)Cc1ccc(C(F)(F)F)cc1 ; ".HCl" ; Cn1cc(C(=O)NC(Cc2ccc(C(F)(F)F)cc2)C(=O)C(N)=O)c(-c2ccccc2F)n1]

### N-(4-amino-3,4-dioxo-1-(4-(Trifluoromethyl)phenyl)butan-2-yl)-3-(2-fluorophenyl)-1-methyl-1H-pyrazole-4-carboxamide (95)

Compound 2-amino-3-(4-(trifluoromethyl)phenyl)propanoic acid was converted to intermediate 95F which was then coupled with 3-(2-fluorophenyl)-1-methyl-1H-pyrazole-4-carboxylic acid using the same conditions as for compound 80 and then further used procedures as described in Example 1 to yield compound 95. Compound 96 (70 mg, yield 55.13%) was obtained as pale yellow solid. ¹H NMR (400 MHz, DMSO-d₆) δ 7.92 (s, 1H), 7.61 (d, J=8.0 Hz, 2H), 7.47-7.37 (m, 2H), 7.34 (d, J=8.0 Hz, 2H), 7.25-7.18 (m, 1H), 7.16-7.09 (m, 1H), 6.96 (br s, 1H), 6.69 (br d, J=6.8 Hz, 1H), 6.22 (br s, 1H), 5.40-5.32 (m, 1H), 3.91 (s, 3H), 3.31 (dd, J=4.6, 14.2 Hz, 1H), 2.97 (dd, J=8.9, 13.9 Hz, 1H). MS (ESI) m/z (M+H)⁺=463.1.

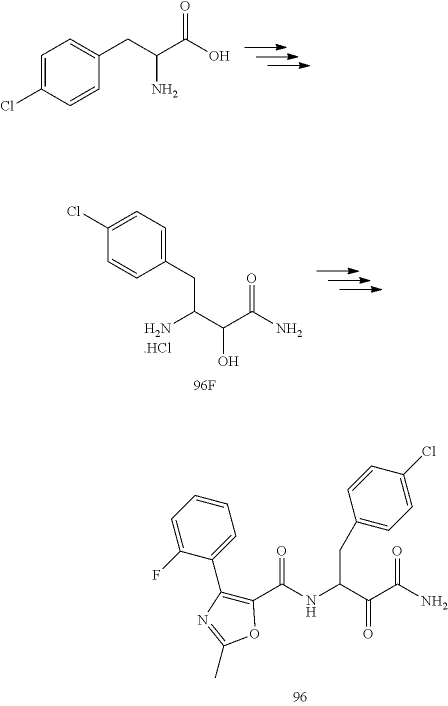 [SMILES: NC(Cc1ccc(Cl)cc1)C(=O)O ; NC(=O)C(O)C(N)Cc1ccc(Cl)cc1 ; ".HCl" ; Cc1nc(-c2ccccc2F)c(C(=O)NC(Cc2ccc(Cl)cc2)C(=O)C(N)=O)o1]

### N-(4-amino-1-(4-chlorophenyl)-3,4-dioxobutan-2-yl)-4-(2-fluorophenyl)-2-methyloxazole-5-carboxamide (96)

Compound 2-amino-3-(4-chlorophenyl)propanoic acid was converted to intermediate 96F which was then coupled with 4-(2-fluorophenyl)-2-methyloxazole-5-carboxylic acid using the same conditions as for compound 80 and then further used procedures as described in Example 1 to yield compound 96. Compound 96 (120 mg, yield 77%) was obtained as white solid. ¹H NMR (DMSO-d₆, 400 MHz): δ 8.86 (d, J=7.6 Hz, 1H), 8.09 (s, 1H), 7.84 (s, 1H), 7.48-7.41 (m, 2H), 7.38-7.33 (m, 2H), 7.31-7.26 (m, 2H), 7.24-7.18 (m, 2H), 5.37-5.26 (m, 1H), 3.16 (dd, J=3.7, 14.2 Hz, 1H), 2.94 (dd, J=10.0, 13.9 Hz, 1H), 2.56 (s, 3H). MS (ESI) m/z (M+H)⁺ 430.1.

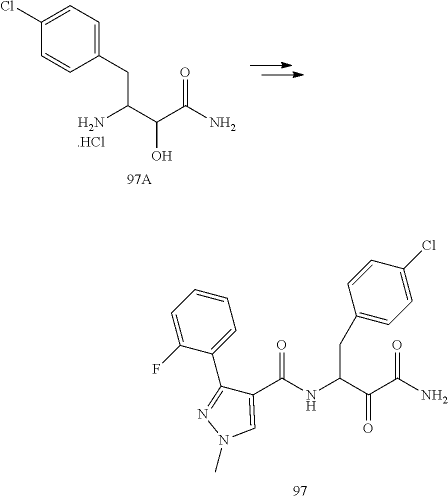 [SMILES: NC(=O)C(O)C(N)Cc1ccc(Cl)cc1 ; ".HCl" ; Cn1cc(C(=O)NC(Cc2ccc(Cl)cc2)C(=O)C(N)=O)c(-c2ccccc2F)n1]

### N-(4-amino-1-(4-chlorophenyl)-3,4-dioxobutan-2-yl)-3-(2-fluorophenyl)-1-methyl-1H-pyrazole-4-carboxamide (97)

Compound 3-amino-4-(4-chlorophenyl)-2-hydroxybutanamide was coupled with 3-(2-fluorophenyl)-1-methyl-1H-pyrazole-4-carboxylic acid using the same conditions as for compound 80 and then further used procedures as described in Example 1 to yield compound 97. Compound 97 (120 mg, yield 65.3%) was obtained as white solid. ¹H NMR (DMSO-d₆, 400 MHz): δ 8.28 (d, J=7.3 Hz, 1H), 8.18 (s, 1H), 8.01 (s, 1H), 7.78 (s, 1H), 7.41-7.30 (m, 4H), 7.28-7.24 (m, 2H), 7.19-7.09 (m, 2H), 5.29-5.11 (m, 1H), 3.91 (s, 3H), 3.11 (dd, J=3.7, 13.9 Hz, 1H), 2.80 (dd, J=10.1, 13.8 Hz, 1H). MS (ESI) m/z (M+H)⁺ 429.1.

### Example 21—Compounds 5 and 8

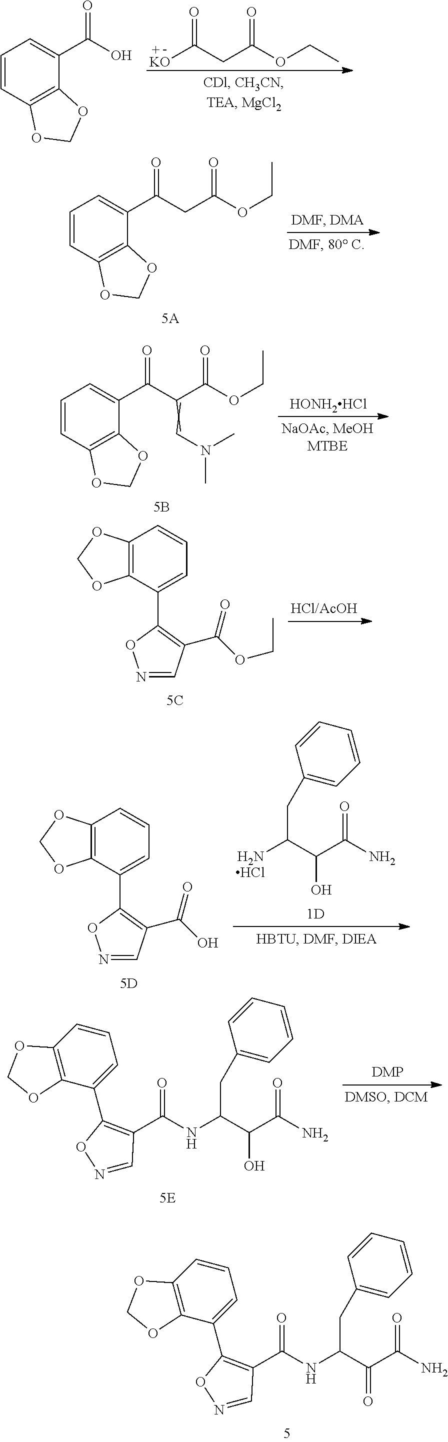

### N-(4-amino-3,4-dioxo-1-phenylbutan-2-yl)-5-(benzo[d][1,3]dioxol-4-yl)isoxazole-4-carboxamide (5)

Flask 1: To a solution of benzo[d][1,3]dioxole-4-carboxylic acid (2 g, 12.04 mmol) in CH₃CN (15 mL) was added CDI (2.19 g, 13.48 mmol). The mixture was stirred at 25° C. for 4 h.

Flask 2: To a solution of ethyl potassium malonate (2.70 g, 15.89 mmol) in CH₃CN (25 mL) was added MgCl₂ (1.15 g, 12.04 mmol) in portions over 15 min. The mixture was stirred at 25° C. for 0.5 h, then TEA (3.65 g, 36.12 mmol) was added and the slurry was stirred for 0.5 h. The solution in flask 1 was transferred to the slurry in flask 2. The mixture was stirred at 25° C. for 18 h. The reaction mixture was quenched with 3N HCl (40 mL) and the solution was concentrated under reduce pressure. The resulting was extracted with MTBE (50 mL×2). The organic layer was washed with H₂O (50 mL), saturated NaHCO₃ (50 mL), saturated NaCl (50 mL), dried over anhydrous Na₂SO₄, filtered and concentrated under reduced pressure to give compound 5A (2.1 g, 73.9% yield) as a yellow oil, which was used for next step without purification.

A mixture of compound 5A (1.1 g, 4.66 mmol) and DMFDMA (2.47 mL, 18.63 mmol) in DMF (15 mL) was stirred at 80° C. for 3 h. The mixture was concentrated under vacuo to give compound 5B (1.2 g, 88.5% yield) as a brown oil, which was used for next step without purification.

NaOAc (676 mg, 8.24 mmol) was added to the mixture of compound 5B (1.20 g, 4.12 mmol) and hydroxylamine hydrochloride (573 mg, 8.24 mmol) in MeOH (7 mL) and MTBE (7 mL). The mixture was stirred at 25° C. for 17 h. The mixture was added saturated NH₄C₁ (20 mL) and extracted with MTBE (20 mL×2). The combined organic phase was washed brine (10 mL), dried over Na₂SO₄, filtered and concentrated under vacuum. The product was purified by FCC (0-10% EA/PE) to give compound 5C (444 mg, 41.3% yield) as a white solid. ¹H NMR (400 MHz, DMSO-d₆) δ 9.07 (s, 1H), 7.29 (d, J=8.0 Hz, 1H), 7.17 (d, J=7.8 Hz, 1H), 7.03-6.97 (m, 1H), 6.13 (s, 2H), 4.24 (q, J=7.1 Hz, 2H), 1.21 (t, J=7.2 Hz, 3H).

HCl (12M, 5 mL) was added to the mixture of compound 5C (244 mg, 0.93 mmol) in AcOH (5 mL). The mixture was stirred at 118° C. for 4.5 h. The mixture was concentrated under vacuum. H₂O (50 mL) was added to the mixture, the mixture was extracted with DCM (50 mL). The organic phase was washed with brine (30 mL), dried over Na₂SO₄, filtered and concentrated under vacuo to give compound 5D (185 mg, 84.9% yield) as a yellow solid, which was used for next step without purification. ¹H NMR (400 MHz, DMSO-d₆) δ 8.96 (s, 1H), 7.28 (d, J=7.7 Hz, 1H), 7.11 (dd, J=1.0, 7.8 Hz, 1H), 6.96 (t, J=7.9 Hz, 1H), 6.14-6.06 (m, 2H).

Compound 5D and intermediate 1D were coupled using the same conditions as for intermediates 58E and 1D and then used procedures as described in Example 1 to yield compound 5. Compound 5 (40 mg, yield 20.8%) was obtained as pale-yellow solid. ¹H NMR (400 MHz, DMSO-d₆) δ 8.88 (br d, J=7.3 Hz, 1H), 8.81 (s, 1H), 8.08 (br s, 1H), 7.82 (br s, 1H), 7.30-7.17 (m, 5H), 7.07 (br dd, J=7.7, 15.7 Hz, 2H), 6.92-6.86 (m, 1H), 6.03-5.86 (m, 2H), 5.31 (br s, 1H), 3.15 (br dd, J=3.4, 13.6 Hz, 1H), 2.81 (br dd, J=10.3, 13.8 Hz, 1H). MS (ESI) m/z (M+H)⁺ 408.1.

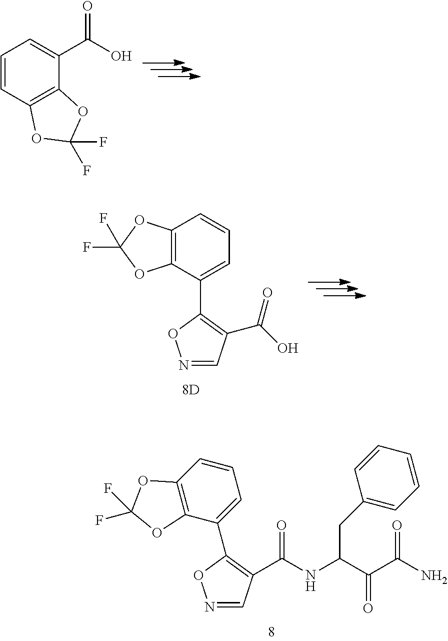 [SMILES: O=C(O)c1cccc2c1OC(F)(F)O2 ; O=C(O)c1cnoc1-c1cccc2c1OC(F)(F)O2 ; NC(=O)C(=O)C(Cc1ccccc1)NC(=O)c1cnoc1-c1cccc2c1OC(F)(F)O2]

### N-(4-amino-3,4-dioxo-1-phenylbutan-2-yl)-5-(2,2-difluorobenzo[d][1,3]dioxol-4-yl)isoxazole-4-carboxamide (8)

Compound 2,2-difluorobenzo[d][1,3]dioxole-4-carboxylic acid was converted to intermediate 8D using procedures as described for compound 5 and then intermediate 8D was coupled with intermediate 1D using procedures as described in compound 58 to yield compound 8. Compound 8 (60 mg, yield 54%) was obtained as white solid. ¹H NMR (DMSO-d₆, 400 MHz): δ. 9.06 (d, J=7.5 Hz, 1H), 8.97 (s, 1H), 8.10 (s, 1H), 7.85 (s, 1H), 7.65-7.47 (m, 2H), 7.36-7.14 (m, 6H), 5.38 (s, 1H), 3.24-3.07 (m, 1H), 2.89-2.75 (m, 1H). MS (ESI) m/z (M+H)⁺ 444.1.

### Example 22—Compounds 11, 27, 30, 29, 45, and 59

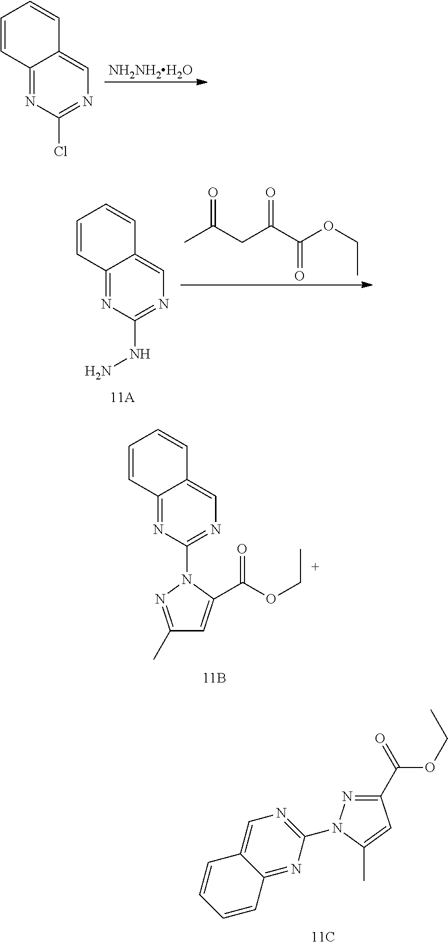

To a mixture of 2-chloroquinazoline (1 g, 6.08 mmol) and K₂CO₃ (1.00 g, 7.24 mmol) was added NH₂NH₂.H₂O (5 mL, 85% purity). The mixture was stirred at 100° C. for 0.5 hr. The reaction mixture was ice cooled and the resulting crude crystals were collected by filtration. The crystals were washed with cold water, air dried to give a residue. The residue was triturated in PE (20 mL) and collected by filtration. Compound 11A (490 mg, yield: 50.4%) was obtained as a yellow solid.

To a solution of compound 11A (490 mg, 3.06 mmol) and ethyl 2,4-dioxopentanoate (484 mg, 3.06 mmol) was added HOAc (5 mL). The mixture was stirred at 100° C. for 16 h. The mixture was concentrated, diluted with EA (25 mL) and filtered. The organic layer was washed with NaHCO₃ (25 mL), brine (25 mL×3), dried over Na₂SO₄, then filtered and concentrated to give a residue. The residue was purified by preparatory-TLC (PE:EA=1:1). Compound 11B (180 mg, yield: 18.1%) was obtained as a yellow oil. Compound 11C (110 mg, yield: 11.3%) was obtained as a yellow oil.

Compound 11B: ¹H NMR (400 MHz, DMSO-d₆) δ 9.69 (s, 1H), 8.23 (d, J=8.4 Hz, 1H), 8.12-8.03 (m, 1H), 8.00-7.93 (m, 1H), 7.78 (dt, J=1.0, 7.6 Hz, 1H), 6.85 (s, 1H), 4.21-4.09 (m, 2H), 2.28 (s, 3H), 1.03 (t, J=7.2 Hz, 3H). MS (ESI) m/z (M+H)⁺ 282.9.

Compound 11C: ¹H NMR (400 MHz, DMSO-d₆) δ 9.79 (d, J=0.7 Hz, 1H), 8.29 (d, J=8.2 Hz, 1H), 8.16-8.05 (m, 2H), 7.83 (ddd, J=1.5, 6.4, 8.1 Hz, 1H), 6.83 (d, J=0.9 Hz, 1H), 4.32 (q, J=7.1 Hz, 2H), 2.68 (d, J=0.9 Hz, 3H), 1.32 (t, J=7.2 Hz, 3H). MS (ESI) m/z (M+H)⁺ 282.9.

### N-(4-amino-3,4-dioxo-1-phenylbutan-2-yl)-3-methyl-1-(quinazolin-2-yl)-1H-pyrazole-5-carboxamide (11)

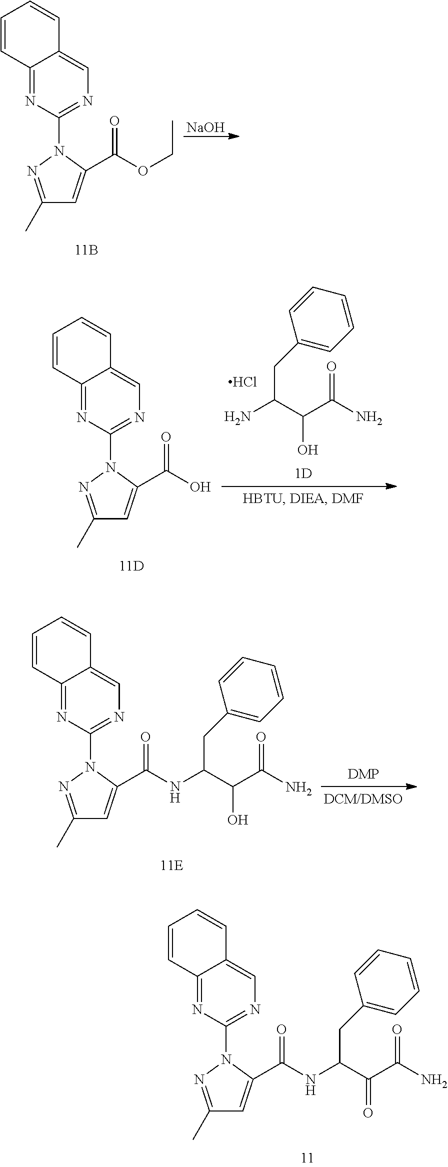

Compound 11B was subjected to procedures as used for converting intermediate 58D to compound 58 as described in Example 19 to yield compound 11. Compound 11 (45 mg, yield 41.4%) was obtained as pale yellow solid, ¹H NMR (400 MHz, DMSO-d₆) δ 9.51 (s, 1H), 9.11 (d, J=7.7 Hz, 1H), 8.19 (d, J=8.2 Hz, 1H), 8.09-7.98 (m, 2H), 7.88-7.79 (m, 2H), 7.75 (t, J=7.6 Hz, 1H), 7.28-7.16 (m, 5H), 6.58 (s, 1H), 5.43-5.15 (m, 1H), 3.13 (dd, J=3.1, 14.1 Hz, 1H), 2.83 (dd, J=9.9, 13.9 Hz, 1H), 2.28 (s, 3H). MS (ESI) m/z (M+H)⁺ 429.1.

### N-(4-amino-3,4-dioxo-1-phenylbutan-2-yl)-5-methyl-1-(quinazolin-2-yl)-1H-pyrazole-3-carboxamide (27)

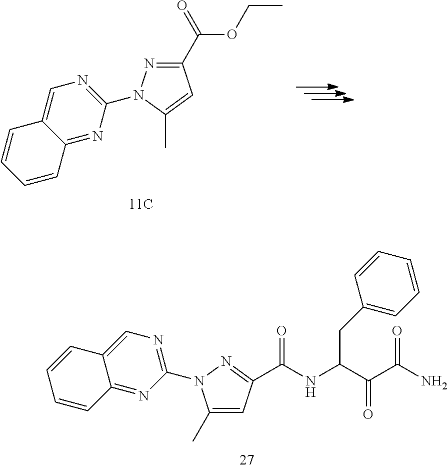 [SMILES: CCOC(=O)c1cc(C)n(-c2ncc3ccccc3n2)n1 ; Cc1cc(C(=O)NC(Cc2ccccc2)C(=O)C(N)=O)nn1-c1ncc2ccccc2n1]

Compound 11C was subjected to procedures as used for converting intermediate 58D to compound 58 as described in Example 19 to yield compound 27. Compound 27 (28 mg, yield 77.1%) was obtained as pale yellow solid, ¹H NMR (400 MHz, DMSO-d₆) δ 9.76 (s, 1H), 8.48 (d, J=7.5 Hz, 1H), 8.26 (d, J=8.2 Hz, 1H), 8.15-7.98 (m, 3H), 7.89-7.73 (m, 2H), 7.27-7.19 (m, 4H), 7.19-7.11 (m, 1H), 6.68 (s, 1H), 5.56-5.29 (m, 1H), 3.24-3.00 (m, 2H), 2.64 (s, 3H). MS (ESI) m/z (M+H)⁺ 429.2.

### N-(4-amino-3,4-dioxo-1-phenylbutan-2-yl)-3-methyl-1-(5-phenylpyrimidin-2-yl)-1H-pyrazole-5-carboxamide (30)

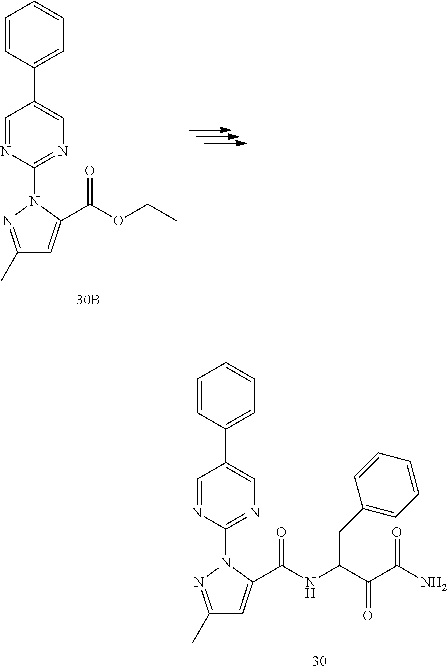 [SMILES: CCOC(=O)c1cc(C)nn1-c1ncc(-c2ccccc2)cn1 ; Cc1cc(C(=O)NC(Cc2ccccc2)C(=O)C(N)=O)n(-c2ncc(-c3ccccc3)cn2)n1]

Compound 30B was prepared from 2-chloro-5-phenylpyrimidine using procedures as described for compound 11. Then, compound 30B was subjected to procedures as used for converting intermediate 58D to compound 58 as described in Example 19 to yield compound 30. Compound 30 (130 mg, yield 82.9%) was obtained as white solid, ¹H NMR (400 MHz, DMSO-d₆) δ 9.07 (d, J=7.2 Hz, 1H), 9.01 (s, 2H), 8.06 (s, 1H), 7.84-7.79 (m, 3H), 7.58-7.44 (m, 3H), 7.28-7.21 (m, 4H), 7.15-7.10 (m, 1H), 6.58 (s, 1H), 5.29-5.21 (m, 1H), 3.18-3.10 (m, 1H), 2.88-2.78 (m, 1H), 2.26 (s, 3H). MS (ESI) m/z (M+H)⁺ 455.1.

### N-(4-amino-3,4-dioxo-1-phenylbutan-2-yl)-5-methyl-1-(5-phenylpyrimidin-2-yl)-1H-pyrazole-3-carboxamide (29)

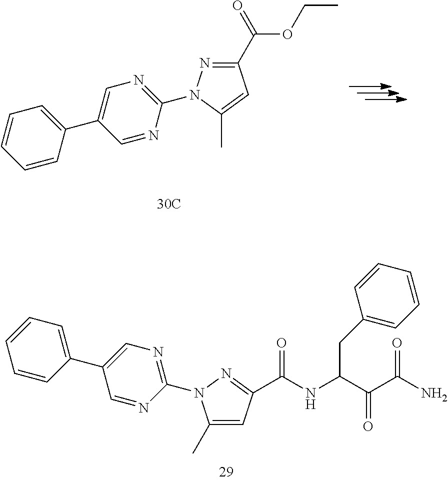 [SMILES: CCOC(=O)c1cc(C)n(-c2ncc(-c3ccccc3)cn2)n1 ; Cc1cc(C(=O)NC(Cc2ccccc2)C(=O)C(N)=O)nn1-c1ncc(-c2ccccc2)cn1]

Compound 30C was prepared from 2-chloro-5-phenylpyrimidine using procedures as described for compound 11. Then, compound 30C was subjected to procedures as used for converting intermediate 58D to compound 58 as described in Example 19 to yield compound 29. Compound 29 (50 mg, yield 33.8%) was obtained as white solid, ¹H NMR (400 MHz, DMSO-d₆) δ 9.25 (s, 2H), 8.12 (br s, 1H), 7.90-7.47 (m, 7H), 7.33-7.15 (m, 5H), 6.69 (s, 1H), 5.56-5.42 (m, 1H), 3.35-3.12 (m, 2H), 2.65 (s, 3H). MS (ESI) m/z (M+H)⁺ 455.2.

### N-(4-amino-3,4-dioxo-1-phenylbutan-2-yl)-3-methyl-1-(4-phenylpyrimidin-2-yl)-1H-pyrazole-5-carboxamide (45)

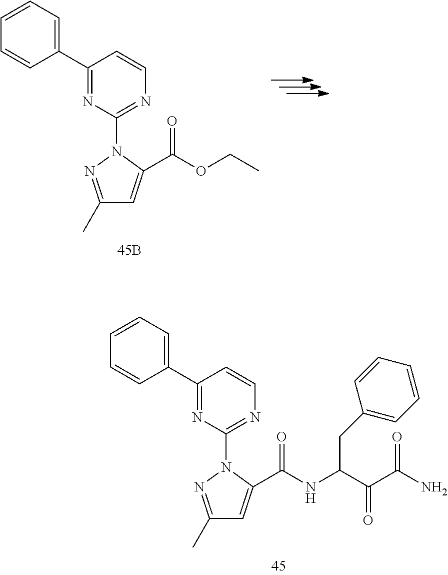 [SMILES: CCOC(=O)c1cc(C)nn1-c1nccc(-c2ccccc2)n1 ; Cc1cc(C(=O)NC(Cc2ccccc2)C(=O)C(N)=O)n(-c2nccc(-c3ccccc3)n2)n1]

Compound 45B was prepared from 2-chloro-4-phenylpyrimidine using procedures as described for compound 11. Then, compound 45B was subjected to procedures as used for converting intermediate 58D to compound 58 as described in Example 19 to yield compound 45. Compound 45 (110 mg, yield 73.5%) was obtained as white solid, ¹H NMR (DMSO-d₆, 400 MHz): δ 9.06 (d, J=7.3 Hz, 1H), 8.78 (d, J=5.3 Hz, 1H), 8.12-8.05 (m, 3H), 8.00 (d, J=5.3 Hz, 1H), 7.83 (s, 1H), 7.58-7.46 (m, 3H), 7.25-7.13 (m, 5H), 6.55 (s, 1H), 5.44-5.36 (m, 1H), 3.11 (dd, J=3.9, 14.0 Hz, 1H), 2.76 (dd, J=9.9, 13.9 Hz, 1H), 2.28 (s, 3H). MS (ESI) m/z (M+H)⁺ 455.2.

### N-(4-amino-3,4-dioxo-1-phenylbutan-2-yl)-5-methyl-1-(4-phenylpyrimidin-2-yl)-1H-pyrazole-3-carboxamide (59)

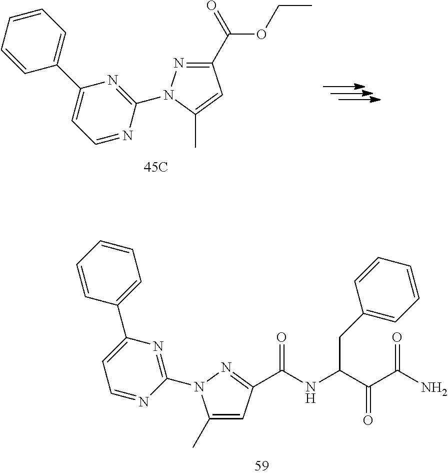 [SMILES: CCOC(=O)c1cc(C)n(-c2nccc(-c3ccccc3)n2)n1 ; Cc1cc(C(=O)NC(Cc2ccccc2)C(=O)C(N)=O)nn1-c1nccc(-c2ccccc2)n1]

Compound 45C was prepared from 2-chloro-4-phenylpyrimidine using procedures as described for compound 11. Then, compound 45C was subjected to procedures as used for converting intermediate 58D to compound 58 as described in Example 19 to yield compound 59. Compound 59 (25 mg, yield 11.9%) was obtained as yellow solid, ¹H NMR (DMSO-d₆, 400 MHz): δ 8.99 (d, J=5.3 Hz, 1H), 8.29-8.25 (m, 2H), 8.10 (br d, J=5.3 Hz, 2H), 7.82 (br s, 1H), 7.65-7.58 (m, 4H), 7.31-7.24 (m, 4H), 7.23-7.17 (m, 1H), 6.72-6.68 (m, 1H), 5.49 (dt, J=4.9, 8.1 Hz, 1H), 3.29 (dd, J=4.9, 14.2 Hz, 1H), 3.16 (br d, J=5.5 Hz, 1H), 2.71-2.69 (m, 3H). MS (ESI) m/z (M+H)⁺ 455.1.

### Example 23—Compounds 43-44

### Methyl 4-(4-(7,9-dioxo-6,10-dioxaspiro[4.5]decan-8-ylidene)-λ³-iodanyl)phenyl)-1,2,5-thiadiazole-3-carboxylate (43)

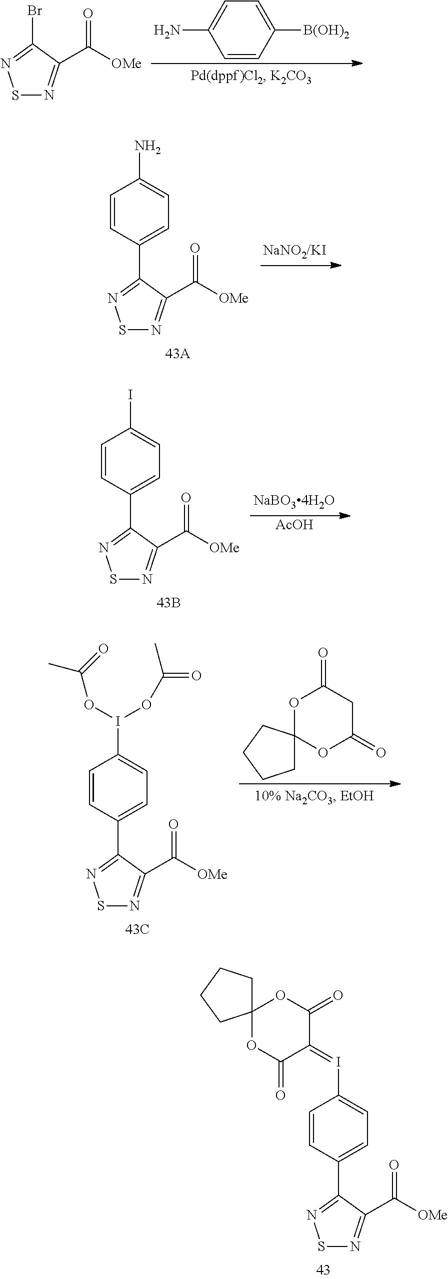

To a solution of methyl 4-bromo-1,2,5-thiadiazole-3-carboxylate (2 g, 8.97 mmol) and (4-aminophenyl)boronic acid (1.60 g, 11.66 mmol) in dioxane (25 mL) and H₂O (2 mL) was added K₂CO₃ (3.72 g, 26.90 mmol), Pd(dppf)Cl₂ (656 mg, 896.67 umol) was added under N₂ atmosphere, the mixture was stirred at 80° C. for 18 h under N₂ atmosphere. The reaction mixture was concentrated to remove solvent, then diluted with EA (50 mL) and filtered; the organic layers were concentrated to give a residue. The residue was purified by flash silica gel chromatography (ISCO®; 12 g SepaFlash® Silica Flash Column, Eluent of 0-30% Ethyl acetate/Petroleum ethergradient @ 30 mL/min). Compound 43A (1.3 g, yield: 61.6%) as light yellow solid was obtained. ¹H NMR (400 MHz, DMSO-d₆) δ 7.47-7.33 (m, 2H), 6.65-6.56 (m, 2H), 5.64 (s, 2H), 3.94-3.85 (m, 3H). MS (ESI) m/z (M+H)⁺ 236.1.

A solution of TsOH.H₂O (2.63 g, 13.81 mmol) in H₂O (20 mL) was added a suspension of compound 43A (1.3 g, 5.53 mmol) in CH₃CN (30 mL) at 0° C., the mixture was stirred for 30 min, then a solution of NaNO₂ (572 mg, 8.29 mmol) in H₂O (10 mL) and KI (1.38 g, 8.29 mmol) in H₂O (10 mL) was added dropwise to the mixture at 0° C., After addition, the mixture was stirred at 25° C. for 16 h. The mixture was quenched by the addition of saturated Na₂SO₃ (˜20 mL) at 0° C. The mixture was concentrated in vacuum to remove CH₃CN. The reaction was filtered, the filter cake was dried in vacuo. The residue was purified by flash silica gel chromatography (ISCO®; 12 g SepaFlash® Silica Flash Column, Eluent of 0-10% Ethyl acetate/Petroleum ethergradient @ 30 mL/min). Compound 43B (1.4 g, yield: 73.2%) as white solid was obtained. ¹H NMR (400 MHz, CDCl₃) δ 7.90-7.77 (m, 2H), 7.52-7.37 (m, 2H), 4.02-3.92 (s, 3H).

Sodium perborate tetrahydrate (4 g, 26.00 mmol) was added in portions to a solution of compound 43B (900 mg, 2.60 mmol) in AcOH (15 mL), the mixture was stirred at 50° C. for 10 h. The reaction mixture was diluted with DCM (50 mL), filtered, the filtrate was diluted with water (100 mL), and extracted three times with DCM (40 mL×2). The combined organic extracts were dried with Na₂SO₄, filtered, and concentrated to give a residue. The residue was triturated in DCM:PE (1:15) (20 mL×3). Filtered and the cake was obtained. Compound 43C (590 mg, yield: 48.9%) as light yellow solid was obtained. ¹H NMR (400 MHz, CDCl₃) δ 8.26-8.15 (m, 2H), 7.89-7.84 (m, 2H), 4.05-3.98 (m, 3H), 2.10-2.00 (m, 6H).

To a solution of compound 43C (590 mg, 1.27 mmol) in EtOH (20 mL) was added the solution of Na₂CO₃ (539 mg, 5.08 mmol) in H₂O (10 mL), then 6,10-dioxaspiro[4.5]decane-7,9-dione (281 mg, 1.65 mmol) was added, the mixture was stirred at 20° C. for 1 h. The reaction mixture was then diluted with water (80 mL), and extracted with DCM (50 mL×3). The combined organic extracts were dried with anhydrous Na₂SO₄, filtered, and concentrated to give a residue. The residue was purified by flash silica gel chromatography (ISCO®; 4 g SepaFlash® Silica Flash Column, Eluent of 0-100% Ethyl acetate/Petroleum ethergradient @ 20 mL/min). The product (Part of Methyl ester was changed to Ethyl ester) was dissolved in MeOH (20 mL), then a solution of Na₂CO₃ (100 mg) in H₂O (2 mL) was added, the mixture was stirred at 20° C. for 4 h. The reaction mixture was then diluted with water (50 mL), and extracted with DCM (30 mL×3). The combined organic layers were dried with anhydrous Na₂SO₄, filtered, and concentrated to give the desired product. Compound 43 (130 mg, yield: 19.9%) as light yellow solid was obtained. ¹H NMR (400 MHz, CDCl₃) δ 8.02-7.92 (m, 2H), 7.86-7.75 (m, 2H), 4.04-3.90 (m, 3H), 2.24-2.15 (m, 4H), 1.85-1.78 (m, 4H). MS (ESI) m/z (M+Na)⁺537.0.

### Ethyl 3-(4-(7,9-Dioxo-6,10-dioxaspiro[4.5]decan-8-ylidene)-λ³-iodanyl)phenyl)-1-methyl-1H-pyrazole-4-carboxylate (44)

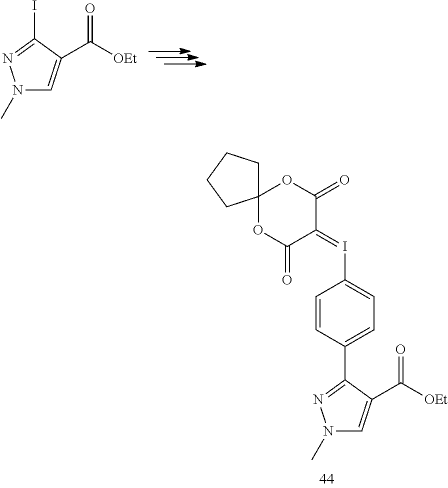 [SMILES: CCOC(=O)c1cn(C)nc1I ; CCOC(=O)c1cn(C)nc1-c1ccc(I=C2C(=O)OC3(CCCC3)OC2=O)cc1]

Compound ethyl 3-iodo-1-methyl-1H-pyrazole-4-carboxylate was converted to the compound 44 using procedures described for compound 43. Compound 44 (120 mg, yield 57.5%) was obtained as pale yellow solid, ¹H NMR (400 MHz, CDCl₃) δ 7.98 (s, 1H), 7.91 (s, 4H), 4.25 (q, J=7.1 Hz, 2H), 3.97 (s, 3H), 2.16 (t, J=7.4 Hz, 4H), 1.82-1.77 (m, 4H), 1.29 (t, J=7.2 Hz, 3H). MS (ESI) m/z (M+Na)⁺546.9.

### Example 24—Compounds 56 and 66

### ethyl-4-(4-((7,9-Dioxo-6,10-dioxaspiro[4.5]decan-8-ylidene)-λ³-iodanyl)phenyl)-2-methyloxazole-5-carboxylate (56)

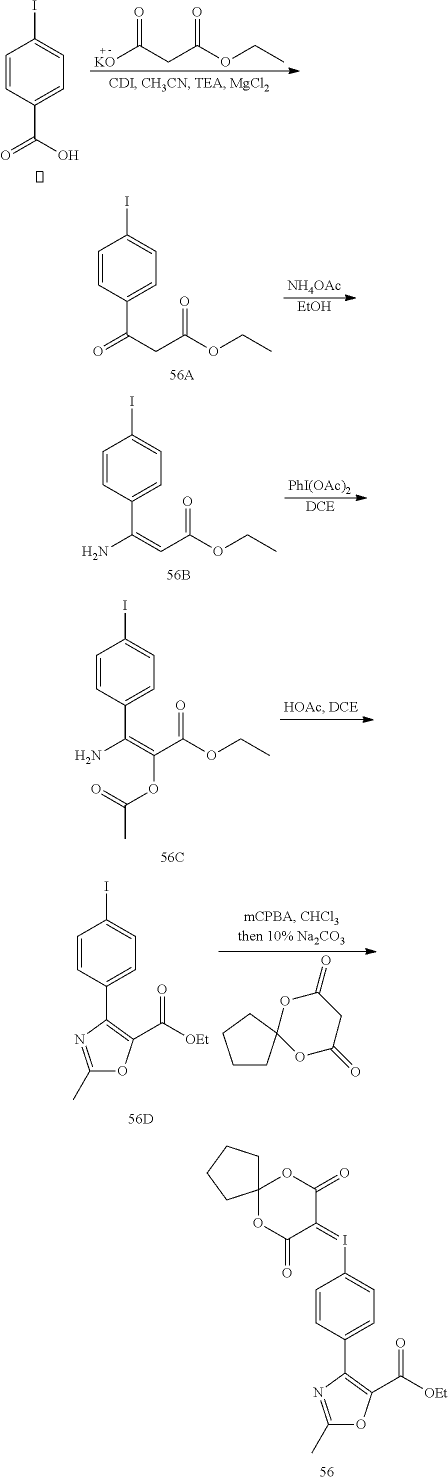

(Flask A) To a solution of 4-iodobenzoic acid (25 g, 100.80 mmol) in CH₃CN (300 mL) was added CDI (18.5 g, 114.09 mmol), the mixture was stirred at 20° C. for 2 h. At the same time, in Flask B to a solution of potassium; 3-ethoxy-3-oxo-propanoate (22.30 g, 131.04 mmol) in CH₃CN (300 mL) was added MgCl₂ (10.6 g, 111.33 mmol) and TEA (301.75 mmol, 42 mL), the mixture was stirred at 20° C. for 2 h. The solution of flask A was then transferred to flask B, the mixture was stirred for 18 h at 20° C. The reaction mixture was diluted with H₂O (200 mL), adjusted to pH ˜4 with HCl (4M), extracted with EA (300 mL×3) and the organic layers were combined and washed with NaHCO₃ (aq) (500 mL), brine (500 mL). And then the organic phase was dried over anhydrous sodium sulfate, filtered and concentrated to give a residue. Compound 56A (31.5 g, yield: 98.2%) as yellow oil was obtained, which was used into the next step without further purification. ¹H NMR (400 MHz, CDCl₃) δ 7.91-7.73 (m, 2H), 7.70-7.42 (m, 2H), 4.30-4.15 (m, 2H), 3.97-3.89 (m, 2H), 1.30-1.19 (m, 3H).

To a solution of compound 56A (31.5 g, 99.02 mmol) in EtOH (300 mL) was added NH₄OAc (20 g, 259.46 mmol), then the mixture was stirred at 85° C. for 18 h. The reaction mixture was concentrated to remove solvent, then diluted with water (150 mL) and extracted with EA (100 mL×3), the organic layers were washed with saturated NaHCO₃ (100 mL×2), dried over Na₂SO₄, filtered and concentrated to give a residue. The residue was purified by flash silica gel chromatography (ISCO®; 220 g SepaFlash® Silica Flash Column, Eluent of 0-10% Ethyl acetate/Petroleum ethergradient @ 100 mL/min). Compound 56B (26 g, yield: 71.5%) as light yellow solid was obtained. ¹H NMR (400 MHz, DMSO-d₆) δ 7.86-7.75 (m, 2H), 7.44-7.34 (m, 2H), 4.77 (s, 1H), 4.05 (q, J=7.1 Hz, 2H), 1.19 (t, J=7.2 Hz, 3H). MS (ESI) m/z (M+H)⁺ 317.9.

To a solution of compound 56B (2 g, 6.31 mmol) in DCE (20 mL) was added PhI(OAc)₂ (2.44 g, 7.57 mmol) in portions at 0° C., then the mixture was stirred at 20° C. for 1 h. The mixture was cooled to 0° C., washed with saturated NaHCO₃ (80 mL), the aqueous phase was extracted with DCM (30 mL), the organic layer was collected, washed with H₂O (50 mL), then dried over Na₂SO₄, filtered and concentrated to give a residue. The residue was purified by flash silica gel chromatography (ISCO®; 20 g SepaFlash® Silica Flash Column, Eluent of 0-10% Ethyl acetate/Petroleum ethergradient @ 30 mL/min). Compound 56C (220 mg, yield: 8.2%) as light yellow oil was obtained. ¹H NMR (400 MHz, DMSO-d₆) δ 7.84-7.80 (m, 2H), 7.16-7.12 (m, 2H), 4.13-4.06 (m, 2H), 1.88 (s, 3H), 1.19-1.15 (m, 3H). MS (ESI) m/z (M+H)⁺ 376.0.

The solution of compound 56C (220 mg, 586.42 umol) in AcOH (2 mL) and DCE (1 mL) was stirred at 90° C. for 1 h. The solvent was removed in vacuo. The residue was dissolved in EtOAc (30 mL), washed with saturated NaHCO₃ (30 mL). The organics were collected and concentrated to give a residue. The residue was purified by preparatory-TLC (PE:EA=5:1). Compound 56D (110 mg, yield: 52.5%) as light yellow solid was obtained. ¹H NMR (400 MHz, CDCl₃) δ 7.86-7.72 (m, 4H), 4.39 (q, J=7.1 Hz, 2H), 2.57 (s, 3H), 1.38 (t, J=7.2 Hz, 3H)

To a solution of compound 56D (0.4 g, 1.12 mmol) in CHCl₃ (8 mL) was added m-CPBA (314 mg, 1.46 mmol, 80% purity), the mixture was stirred at 20° C. for 18 h. The mixture was concentrated to get rid of most of solvent to give a residue. The residue was dissolved in EtOH (15 mL), and the reaction was added Na₂CO₃ (475 mg, 4.48 mmol) in H₂O (10 mL), and then added 6,10-dioxaspiro[4.5]decane-7,9-dione (248 mg, 1.46 mmol) quickly. The reaction mixture was then stirred at 20° C. for 2 h. The residue was diluted with water (100 mL) and extracted with EA (50 mL×2). The combined organic extracts were washed with brine (100 mL) and dried with anhydrous Na₂SO₄, filtered and concentrated to give a residue. The residue was purified by flash silica gel chromatography (ISCO®; 12 g SepaFlash® Silica Flash Column, Eluent of 0-100% Ethyl acetate/Petroleum ethergradient @ 30 mL/min). Compound 56 (190 mg, yield: 30.7%) was obtained as white solid. ¹H NMR (400 MHz, CDCl₃) δ 8.25-8.09 (m, 2H), 7.97-7.85 (m, 2H), 4.40 (q, J=7.1 Hz, 2H), 2.59 (s, 3H), 2.21-2.12 (m, 4H), 1.84-1.75 (m, 4H), 1.39 (t, J=7.2 Hz, 3H). MS (ESI) m/z (M+Na)⁺548.1.

### ethyl-4-(2-((7,9-dioxo-6,10-dioxaspiro[4.5]decan-8-ylidene)-λ³-iodanyl)phenyl)-2-methyloxazole-5-carboxylate (66)

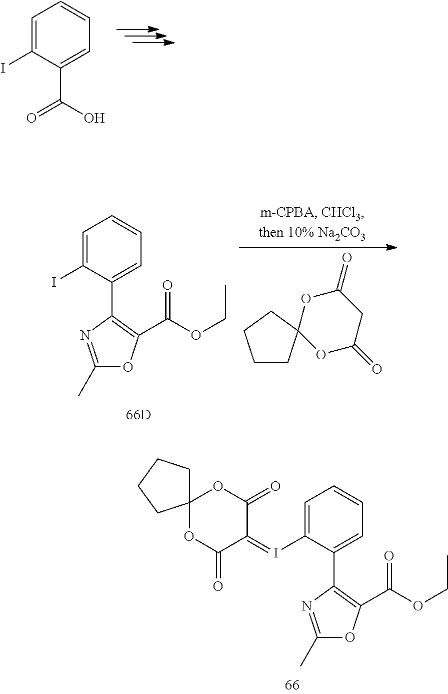

Compound 2-iodobenzoic acid was converted to intermediate 66D using the same procedures as described for synthesis of intermediate 58D. Further, intermediate 66D was treated with 6,10-dioxaspiro[4.5]decane-7,9-dione using the same conditions as described for compound 56 to obtain final compound 66. Compound 66 (90 mg, yield 15.3%) was obtained as white solid, ¹H NMR (CDCl₃, 400 MHz): δ 8.77 (dd, J=1.8, 7.8 Hz, 1H), 7.67 (dd, J=1.1, 8.2 Hz, 1H), 7.61-7.55 (m, 1H), 7.54-7.48 (m, 1H), 4.46 (q, J=7.1 Hz, 2H), 2.66 (s, 3H), 2.29-2.21 (m, 4H), 1.85 (td, J=3.9, 7.1 Hz, 4H), 1.43 (t, J=7.2 Hz, 3H). MS (ESI) m/z (M+H)⁺ 548.0.

### Example 25—Compound 103

### N-(4-amino-3,4-dioxo-1-phenylbutan-2-yl)-3-(1-isopropyl-2-oxo-2,3-dihydro-1H-benzo[d]imidizol-4-yl)-1-methyl-1H-pyrazole-4-carboxamide (103)

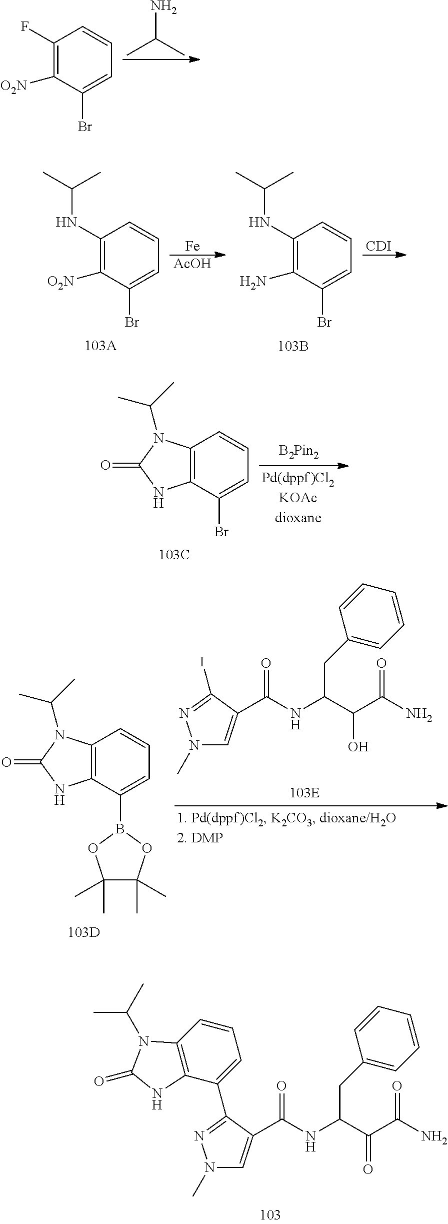

The solution of 1-bromo-3-fluoro-2-nitrobenzene (4.5 g, 20.45 mmol) and isopropyl amine (1.21 g, 20.45 mmol) in EtOH (20 mL) was stirred at 50° C. for 48 h. The solvent was removed in vacuo. The residue was purified by column (PE:EA=10:1) to give compound 103A (5 g, yield: 94.34%) as brown oil.

To a solution of compound 103A (5 g, 19.30 mmol) in AcOH (60 mL) was added Fe (5.39 g, 96.49 mmol). The mixture was stirred at 60° C. for 1 h. The solvent was removed in vacuo. The residue was washed with saturated NaHCO₃ (200 mL), extracted with EtOAc (100 mL×2). The organics were collected, washed with brine (200 mL), dried with Na₂SO₄, filtered, and concentrated to give compound 103B (4.4 g, crude) as brown oil, which was used directly for the next step without further.

To a solution of compound 103B (4.4 g, 19.20 mmol) in THF (60 mL) was added TEA (5.4 mL, 38.41 mmol), CDI (6.23 g, 38.41 mmol). The mixture was stirred at 20° C. for 12 h. The mixture was washed with H₂O (50 mL), extracted with EtOAc (50 mL×2). The organics were collected and concentrated. The residue was purified by column (PE:EA=2:1) to give compound 103C (2.5 g, yield: 51.03%) as brown solid.

To a solution of compound 103C (400 mg, 1.57 mmol) and 4,4,4′,4′,5,5,5′,5′-octamethyl-2,2′-bi(1,3,2-dioxaborolane) (B₂Pin₂) (398 mg, 1.57 mmol) in dioxane (10 mL) was added Pd(dppf)Cl₂ (115 mg, 156.79 umol), KOAc (462 mg, 4.70 mmol). The mixture was stirred at 90° C. for 12 h under N₂. The solution was filtered. The filtrate was collected and concentrated. The residue was purified by column (PE:EA=2:1) to give compound 103D (398 mg, yield: 84.00%) as light brown solid)

Compounds 103D and intermediate 103E were converted to compound 103 using procedures as described in Example 1. Compound 103 (70 mg, yield: 64.6%) as a white solid was obtained. ¹H NMR (DMSO-d₆, 400 MHz): δ 9.96 (br s, 1H), 8.07 (s, 1H), 7.92-7.46 (m, 3H), 7.35-7.11 (m, 8H), 6.97-6.92 (m, 1H), 5.37-5.31 (m, 1H), 4.65-4.57 (m, 1H), 3.94 (s, 3H), 3.21-3.16 (m, 1H), 2.90-2.84 (m, 1H), 1.49 (d, J=7.2 Hz, 6H). MS (ESI) m/z (M+H)⁺ 475.2.

### Example 26—Compounds 93 and 104

### N-(1-oxo-3-phenyl-1-(1H-tetrazol-5-yl)propan-2-yl)-4-phenyl-1,2,5-thiadiazole-3-carboxamide (93)

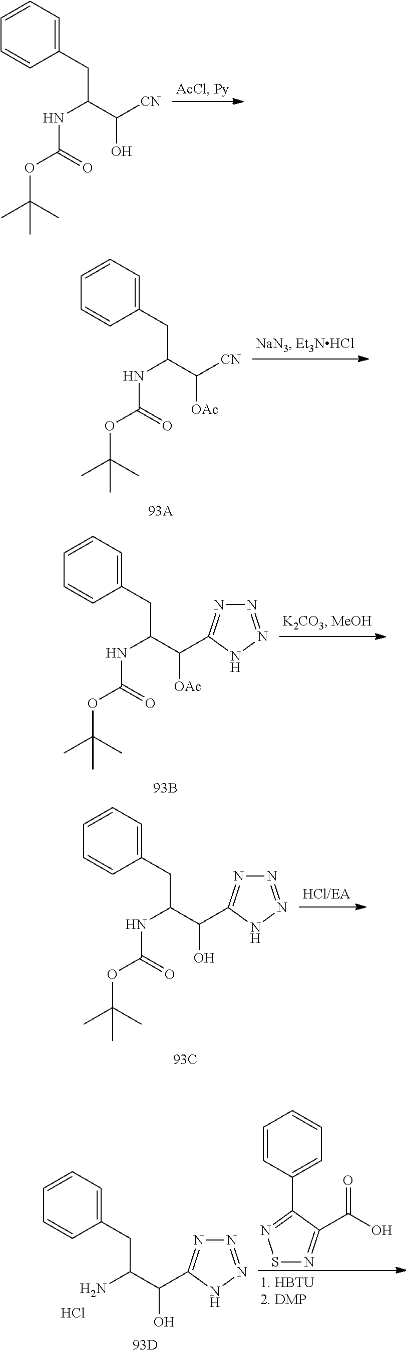

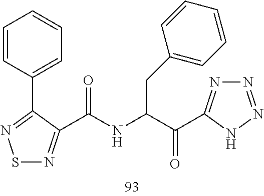 [SMILES: O=C(NC(Cc1ccccc1)C(=O)c1nnn[nH]1)c1nsnc1-c1ccccc1]

To a solution of tert-butyl (1-cyano-1-hydroxy-3-phenylpropan-2-yl)carbamate (1 g, 3.62 mmol) in DCM (15 mL) was added Pyridine (6.19 mmol, 0.5 mL), then acetyl chloride (5.61 mmol, 0.4 mL) was added dropwise, the mixture was stirred at 10° C. for 20 h. The reaction mixture was diluted with DCM (20 mL) and water (50 mL), the aqueous phase was extracted with DCM (20 mL×2), the organic layers were washed with 1N HCl (30 mL), sat. NaHCO₃ (30 mL) and brine (50 mL), dried over Na₂SO₄, filtered and concentrated to give a residue. Compound 93A (1 g, yield: 86.7%) as light yellow oil was obtained, which was used into the next step without further purification. ¹H NMR (400 MHz, CDCl₃) δ 7.36-7.27 (m, 3H), 7.22-7.16 (m, 2H), 5.42-5.33 (m, 1H), 4.70 (d, J=8.5 Hz, 1H), 4.32 (br s, 1H), 3.10-2.83 (m, 2H), 2.16 (s, 3H), 1.40 (s, 9H). MS (ESI) m/z (M+Na)⁺341.1.

To a mixture of compound 93A (500 mg, 1.57 mmol), Et₃N HCl (432 mg, 3.14 mmol) in toluene (15 mL) was added NaN₃ (250 mg, 3.85 mmol), the mixture was stirred at 110° C. for 18 h. The reaction mixture was diluted with toluene (20 mL) and extracted with water (50 mL×3), the combined water layers were acidized with concentrated HCl to pH ˜2, and extracted with EA (30 mL×2), the organic layers were washed with brine (50 mL), dried over Na₂SO₄, filtered and concentrated to give a residue. The residue was triturated in EA (2 mL) and PE (20 mL) twice, filtered and dried in vacuo. Compound 93B (500 mg, yield: 74.4%) as light yellow solid was obtained. ¹H NMR (400 MHz, DMSO-d₆) δ 7.33-7.16 (m, 6H), 7.02 (d, J=9.0 Hz, 1H), 6.01-5.89 (m, 1H), 4.23-4.16 (m, 1H), 2.86-2.64 (m, 2H), 2.21-2.10 (m, 3H), 1.26-1.18 (m, 9H). MS (ESI) m/z (M+H)⁺ 362.2.

To a solution of compound 93B (400 mg, 1.11 mmol) in MeOH (15 mL) was added K₂CO₃ (610 mg, 4.41 mmol) in H₂O (3 mL), the mixture was stirred at 15° C. for 4 h. The reaction mixture was concentrated to remove MeOH, diluted with water (20 mL), extracted with EA (20 mL), the aqueous layer was acidized with concentrated HCl to pH ˜2, extracted with EA (20 mL×2), the organic layers were dried over Na₂SO₄, filtered and concentrated to give a residue. Compound 93C (420 mg, crude) was obtained as light yellow solid, which was used into the next step without further purification. ¹H NMR (400 MHz, DMSO-d₆) δ 7.30-7.16 (m, 6H), 6.56 (d, J=9.0 Hz, 1H), 6.37 (br d, J=4.0 Hz, 1H), 5.02 (t, J=4.5 Hz, 1H), 3.99-3.92 (m, 1H), 2.98-2.57 (m, 2H), 1.24 (s, 9H). MS (ESI) m/z (M+Na)⁺ 342.2.

To a solution of compound 93C (420 mg, 1.32 mmol) in EA (3 mL) was added HCl/EtOAc (4M, 3 mL), the mixture was stirred at 15° C. for 2 h. The reaction mixture was concentrated to give a residue. The residue was triturated in EA (3 mL) and PE (20 mL), filtered and dried in vacuo. Compound 93D (300 mg, yield: 89.2%, HCl) as light yellow solid was obtained. ¹H NMR (400 MHz, DMSO-d₆) δ 8.26 (br s, 3H), 7.39-7.12 (m, 6H), 5.03 (t, J=4.5 Hz, 1H), 3.82 (s, 1H), 3.08-2.91 (m, 2H). MS (ESI) m/z (M+Na)⁺ 276.2

Compounds 93D and 4-phenyl-1,2,5-thiadiazole-3-carboxylic acid were converted to compound 93 using procedures as described in Example 17. Compound 93 (15 mg, yield: 37.7%) as a white solid was obtained. ¹H NMR (400 MHz, DMSO-d₆) δ 9.33 (br dd, J=7.3, 16.8 Hz, 1H), 7.66-7.56 (m, 2H), 7.49-7.42 (m, 1H), 7.42-7.34 (m, 2H), 7.33-7.06 (m, 5H), 5.74-5.67 (m, 1H), 3.16-3.10 (m, 2H). MS (ESI) m/z (M+H)⁺ 406.1.

### N-(1-oxo-3-phenyl-1-(1H-1,2,4-triazol-3-yl)propan-2-yl)-4-phenyl-1,2,5-thiadiazole-3-carboxamide (104)

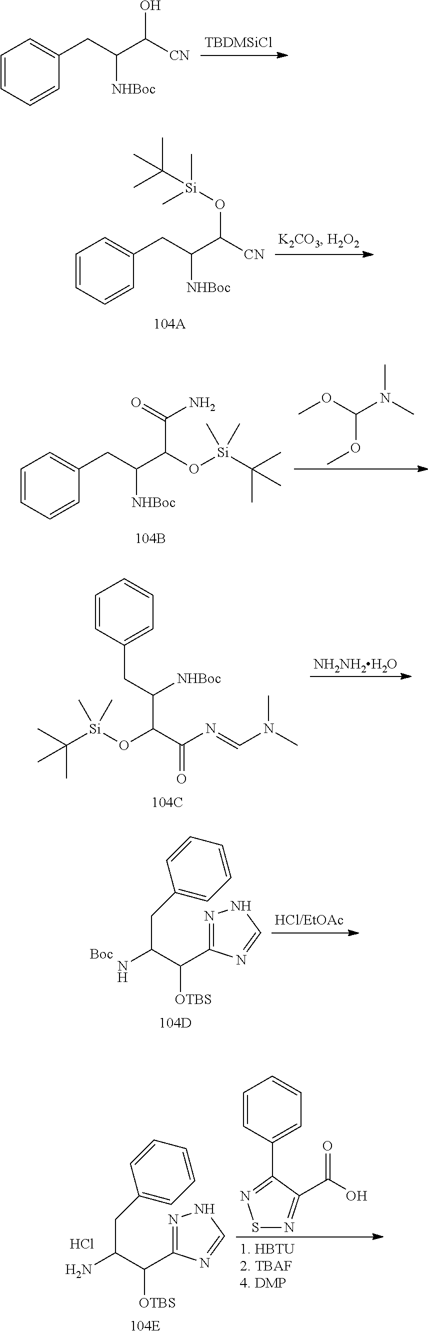

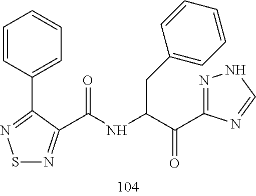 [SMILES: O=C(NC(Cc1ccccc1)C(=O)c1nc[nH]n1)c1nsnc1-c1ccccc1]

To a solution of tert-butyl (1-cyano-1-hydroxy-3-phenylpropan-2-yl)carbamate (500 mg, 1.81 mmol) in DMF (5 mL) was added imidazole (246 mg, 3.62 mmol) and TBDMSiCl (2.90 mmol, 0.35 mL) at 0° C. The mixture was stirred at 25° C. for 12 h. The mixture was diluted with EA (200 mL), washed with brine (200 mL), dried over Na₂SO₄, filtered and concentrated. The residue was purified by column chromatography (SiO₂, Petroleum ether/Ethyl acetate=10/1 to 1/1). Compound 104A (2.9 g) was obtained as a colorless oil. ¹H NMR (400 MHz, CDCl₃) δ 7.36-7.14 (m, 6H), 4.75-4.61 (m, 1H), 4.10-3.97 (m, 1H), 3.20-2.70 (m, 2H), 1.38 (s, 9H), 1.00-0.83 (m, 9H), 0.26-0.08 (m, 6H).

To a solution of compound 104A (450 mg, 1.15 mmol) and K₂CO₃ (318 mg, 2.30 mmol) in DMSO (10 mL) was added H₂O₂ (23.04 mmol, 2.21 mL, 30% purity) at 0° C., the mixture was stirred at 15° C. for 20 h. The reaction mixture was quenched with saturated Na₂S₂O₃ (20 mL) slowly at ice water, diluted with water (30 mL), extracted with EtOAc (30 mL×3), the organic layers were washed with brine (30 mL×2), dried over Na₂SO₄, filtered and concentrated to give a residue. Compound 104B (400 mg, crude) was obtained as colorless oil, which was used into the next step without further purification.

A solution of compound 104B (400 mg, 978.94 umol) in 1,1-dimethoxy-N,N-dimethyl-methanamine (75.28 mmol, 10 mL) was stirred at 30° C. for 1 h. The reaction mixture was diluted with water (50 mL) at ice water, extracted with EA (20 mL×3), the organic layers were washed with brine (30 mL×2), dried over Na₂SO₄, filtered and concentrated to give a residue. Compound 104C (420 mg, crude) was obtained as light yellow oil, which was used into the next step without further purification.

To a solution of compound 104C (410 mg, 884.22 umol) in CH₃COOH (5 mL) was added NH₂NH₂H₂O (884.22 umol, 0.43 mL), the mixture was stirred at 85° C. for 1.5 h. The reaction mixture was diluted with water (60 mL) at ice water, extracted with EA (30 mL×3), the organic layers were washed with brine (80 mL×2), dried over Na₂SO₄, filtered and concentrated to give a residue. Compound 104D (400 mg, crude) was obtained as light yellow oil, which was used into the next step without further purification. MS (ESI) m/z (M+H)⁺ 433.3.

To a solution of compound 104D (400 mg, 924.58 umol) in EA (3 mL) was added HCl/EtOAc (4M, 4.62 mL), the mixture was stirred at 15° C. for 2 h. The reaction mixture was concentrated to give a residue. Compound 104E (350 mg, crude, HCl) was obtained as yellow solid, which was used into the next step without further purification. MS (ESI) m/z (M+H)⁺ 333.2.

Compounds 104E and 4-phenyl-1,2,5-thiadiazole-3-carboxylic acid were coupled using peptide coupling conditions as in Example 17 and then deprotection using TBAF followed by oxidation using procedure for Example 17 to obtain compound 104. Compound 104 (40 mg, yield: 53.5%) as a white solid was obtained. ¹H NMR (400 MHz, CD₃CN) δ 8.45 (s, 1H), 7.83 (d, J=7.1 Hz, 1H), 7.66-7.55 (m, 2H), 7.49-7.36 (m, 3H), 7.34-7.16 (m, 6H), 5.92-5.87 (m, 1H), 3.45 (dd, J=4.6, 14.2 Hz, 1H), 3.12 (dd, J=8.6, 13.9 Hz, 1H). MS (ESI) m/z (M+H)⁺ 405.1.

### EXAMPLE SECTION III

### Example A

### Efficacy of Calpain Inhibitors in a Model of Liver Fibrosis

### CCL4 Model

This study is performed to evaluate the effects of calpain compounds disclosed herein on a Carbon Tetrachloride (CCl4)-induced liver fibrosis in male BALB/c mice. Liver fibrosis is induced in mice by the administration of CCl4 twice weekly for four weeks. CCl4 administered animals are treated with compounds disclosed herein in a therapeutic treatment mode on day 14 post initiation of CCl4 injection and continued until study termination. Study is terminated 96 hours following the last CCl4 administration, i.e., on day 32 post CCl4 administration. Compounds described herein show an improvement in fibrosis score and no effect on liver enzymes, liver weight or body weight compared to vehicle.

### Induction of Liver Fibrosis

Carbon tetrachloride (CCl4) is purchased from Sigma (cat #319961). To induce liver fibrosis, mice are administered CCl4 (1:1, CCl4:mineral oil) via intraperitoneal (ip) injection at a dose volume of 1 ml/kg body weight. On every CCl4 administration day animals are weighed prior to administration of CCl4 and the dose volume is adjusted as per body weight of individual animal. Animals are administered CCl4: mineral oil twice weekly i.e. every Monday and Thursday for four weeks, 2 hours post morning treatment. The final CCl4 injection is received on day 28 post first CCl4 injection.

### Bleeds

Animals are bled via sub-mandibular route on day-4 prior to study initiation, and on day 13, post CCl4 administration, a day before the therapeutic treatment began. Plasma is prepared and stored at −20° C. until it is analyzed. Upon study termination day 32 post CCl4 administration, animals are bled via cardiac puncture. Serum is prepared and stored at −20° C. until shipped to contract company for liver enzyme panel analysis.

### Administration of Compounds

Treatment compounds are prepared in methylcellulose for oral gavage administration. In one example, the following compounds of Formula II-a can be administered:

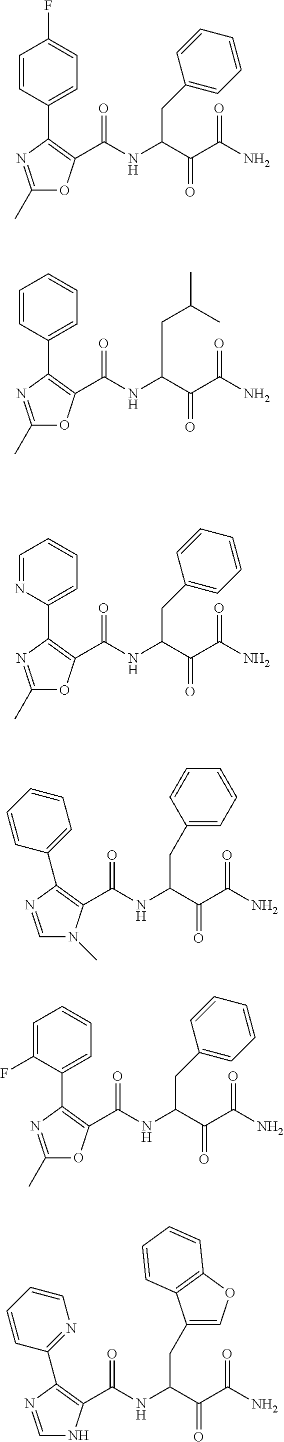 [SMILES: Cc1nc(-c2ccc(F)cc2)c(C(=O)NC(Cc2ccccc2)C(=O)C(N)=O)o1 ; Cc1nc(-c2ccccc2)c(C(=O)NC(CC(C)C)C(=O)C(N)=O)o1 ; Cc1nc(-c2ccccn2)c(C(=O)NC(Cc2ccccc2)C(=O)C(N)=O)o1 ; Cn1cnc(-c2ccccc2)c1C(=O)NC(Cc1ccccc1)C(=O)C(N)=O ; Cc1nc(-c2ccccc2F)c(C(=O)NC(Cc2ccccc2)C(=O)C(N)=O)o1 ; NC(=O)C(=O)C(Cc1coc2ccccc12)NC(=O)c1[nH]cnc1-c1ccccn1]

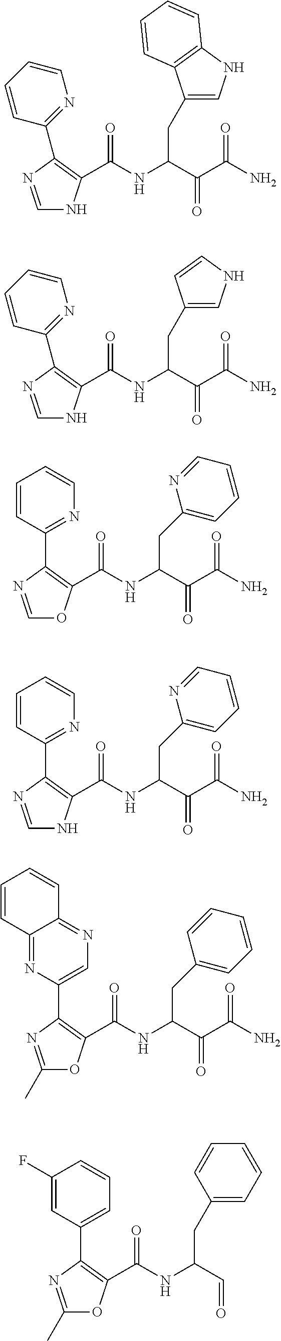

Vehicle (0.5% methylcellulose) is prepared weekly. Treatment compound is prepared once weekly at Aragen and stored at room temperature in the dark. A total of 100 microliters of each compound is administered AM and PM daily via oral route. Animals receive treatment on day 14 post first CCl4 administration and continue until study termination. Animals are harvested within 2 to 4 hours after receiving final treatment.

### Harvest

All surviving animals are humanely euthanized approximately 96 hours following the last CCl4 administration, i.e, on day 32 post CCL4 administration. Median lobe of liver is fixed into 10% NBF for histology, remaining lobes of liver were weighed and snap frozen into two different tubes.

### Histologic Analysis

Picrosirius Red (PSR)-stained slides are examined under polarized light. Birefringence in the section is considered fibrosis and scored according to the following subjective scale: 0=no fibrosis above normal portal areas; 1=minimally increased fibrosis in fine strands between lobules; 2=mildly increased fibrosis in fine strands between lobules and some collagen birefringence in areas of necrosis/mineralization; 3=moderately increased fibrosis in fine strands between lobules and mild birefringence in areas of necrosis/mineralization; 4=markedly increased fibrosis between lobules or in areas of necrosis/mineralization; 5=severely increased fibrosis. Statistical analysis was performed using an unpaired t-test (GraphPad Prism software).

### Clinical Observations and Body Weight

The procedures described above were followed using Compound 405 as a test compound administered at 30 mg/kg BID or 100 mg/kg BID. Body weights were measured prior to every CCl4 administration. CCl4 dose volume was adjusted based on individual body weight. All mice administered CCl4 showed weight loss in the first week of the study then tended to show a gradual increase in body weights during the latter part of the study. The CCl4 administered animals showed significant increase in liver weight compared to saline treated control animals. Average liver weight of 2.24+/−0.06 g was recorded in animals treated with vehicle compared to no CCl4 administered control mice 1.45+/−0.03 g (p<0.0001).

### Fibrosis

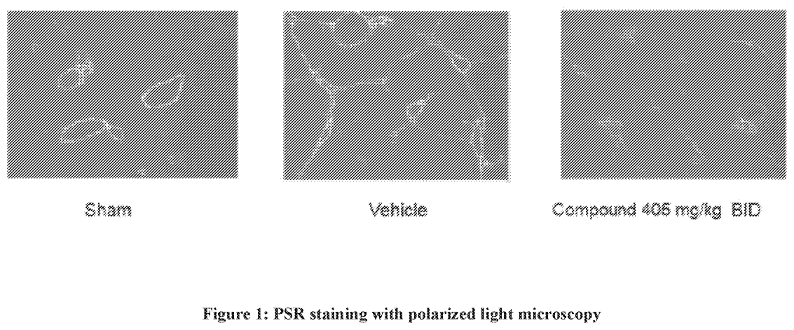

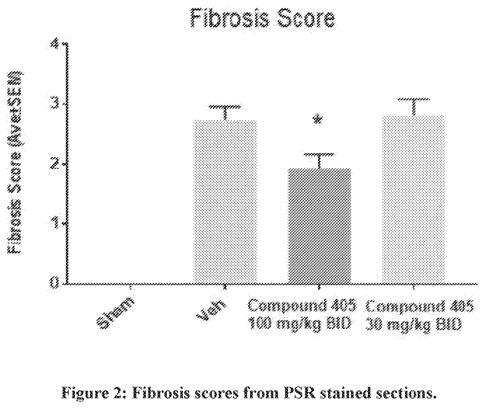

Liver sections stained with PSR and observed under polarized light showed collagen accumulation and crosslinking in the CCl4 treated mice, confirming the induction of fibrosis by Compound 405 (FIG. 1). Treatment with Compound 405 showed a decrease in the length and thickness of the collagen fibers. Sections were analyzed using the scale described in the methods section. FIG. 2 summarizes the scores for each group. A statistically significant reduction in Fibrosis score was observed when CCl4-treated mice were dosed with Compound 405.

Compound 405 dosed therapeutically at 100 mg/kg, twice a day, significantly reduced fibrosis in this model. The results suggest an anti-fibrotic effect of Compound 405 in liver fibrosis. Data suggest that calpain inhibitors can have beneficial effects in different forms of liver fibrosis either by themselves or in combination. Combination therapies may be specially considered in diseases like NASH, where combination of anti-fibrotic agents with anti-inflammatory or agents that modulate the metabolic component could result in maximal benefit.

### Example B

### CAPN1, CAPN2, and CAPN9 Expression in Normal and Diseased Human Liver

Immunohistochemical (IHC) evaluation of CAPN1, CAPN2, and CAPN9 is performed on normal and diseased human liver. Diseases included were Fatty liver, NASH, cirrhosis, PBC and PSC.

### Methodology

CAPN1 is detected using monoclonal antibodies to CAPN1 (Invitrogen/Thermo, clone MA3-940), CAPN2 using (Biorbyt 305855, clone 1381CT669.7.66.71) and CAPN9 (Abnova H00010753-MO0, clone 3A6). All assays are controlled via the detection of abundant, but restricted control proteins (cytokeratins in gastrointestinal mucosa) and the IHC analysis of each section is controlled via non-immune IgGs and ‘no primary’ negative controls. Three assays are completed and in each case, the assay control gives the expected pattern of cytokeratins in mucosal epithelial cells. The non-immune controls generate negligible levels of nonspecific immunoreactivity which does not interfere with the interpretation of specific CAPN-immunoreactivity.

To evaluate antibody specificity, CAPN1, CAPN2 and CAPN9 antibodies are tested using sections containing parental cell lines, or cell lines expressing recombinant human CAPN2 or CAPN9. CAPN1 antibodies do not stain any cell lines under the conditions tested. The CAPN2 and CAPN9 antibodies only labelled the appropriate and expected cell lines.

### Immunohistochemistry

All sections are used at a thickness of 4 μm and all incubations were carried out at ambient temperature unless stated otherwise. The sections were de-paraffinized, antigen retrieved and rehydrated using either pH6 (CAPN1 and CAPN2) or pH9 (CAPN9) Flex Plus 3-in-1 antigen retrieval buffers in a PT Link automated antigen retrieval system at 97° C. for 20 min with automatic heating and cooling. Following antigen retrieval, the slides were placed in Flex buffer (50 mM Tris.HCl, 300 mM NaCl, 0.1% Tween-20, pH 7.6) and allowed to cool. The slides were then loaded into a Dako Autostainer Plus. The sections were then incubated with Flex Plus Peroxidase Blocking reagent, rinsed with Flex buffer followed by an incubation with Protein Block reagent (DAKO, Cat #X0909), which was removed by air-jet. The sections were then incubated with either the primary antibody diluted in DAKO antibody diluent (DAKO, Cat #K8006), the isotype and concentration matched non-immune IgG or antibody diluent alone (no primary). Following incubation with the respective primary antibodies, the sections were rinsed twice in Flex buffer, incubated with Flex plus-HRP secondary, rinsed twice in Flex buffer and then incubated with diaminobenzidine (DAB) substrate. The chromogenic reaction was stopped by rinsing the slides with distilled water. Following chromogenesis, the sections were removed from the Dako Autostainer Plus, counterstained with haematoxylin, dehydrated in an ascending series of ethanol (90-99%), cleared in three changes of xylene and then cover-slipped under DePeX. Assay controls, demonstrating pan cytokeratin (PCK) expression in Colon, were included to validate the anti-mouse Dako EnVision Flex plus-HRP and chromogenic reagents. A ‘no-primary’ control was also included. Stained sections were analyzed, and suitable digital images captured, using an Aperio ScanScope AT Turbo.

### Results

The expression and distribution of CAPN1, 2, and 9 were considerably increased in diseased liver compared with tissues diagnosed as normal. This was especially notable in tissues with fibrotic or degenerative change (NASH, cirrhosis, PBC, PSC). Some sections diagnosed as normal with immunoreactivity often had areas of disease, which showed increase CAPN1 staining (areas with fatty change or necrosis and inflammation). CAPN1 was observed in the widest variety of cell types in normal and diseased tissues, while CAPN 2 and 9 were strongly upregulated mostly in bile duct epithelium.

### CAPN1

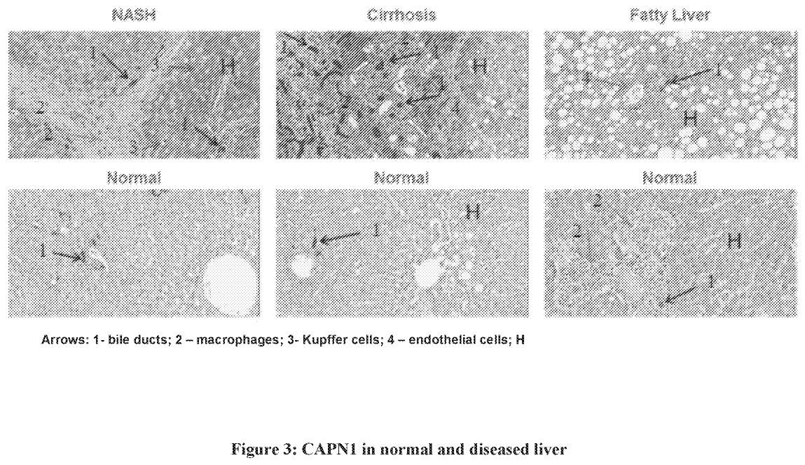

CAPN1 reactivity was increased in disease tissue compare to normal tissue. CAPN1 reactivity was widespread and includes bile duct epithelial cells, Kupfer cells, macrophages and hepatocytes (FIG. 3). Strongest staining was observed in bile duct epithelial cells and Kuppfer cells. A clear increase in immunoreactivity was found in NASH and cirrhosis while the Fatty Liver samples show lower immuno-reactivity. Hepatocyte and endothelial cell stain tended to be strongest in diseased sections. In some tissue diagnosed as normal, areas exhibiting minimal fatty change (FIG. 3, bottom middle), and areas of necrosis and inflammation (FIG. 3, bottom right) show increasing CAPN1 hepatocyte staining.

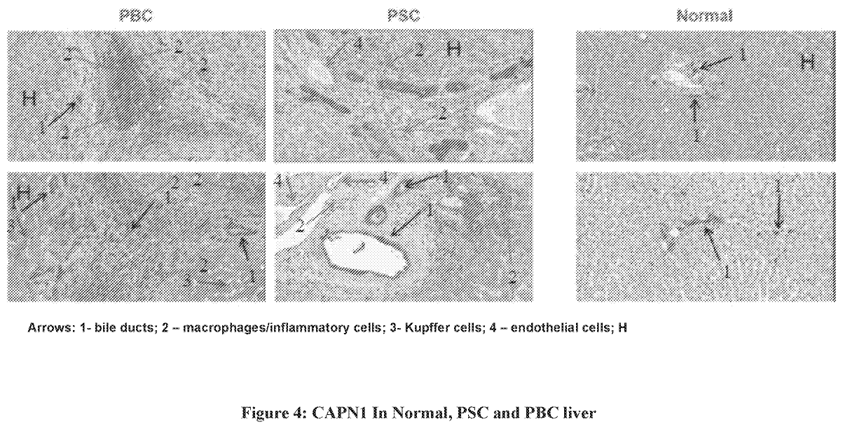

CAPN1 reactivity was increased in PBC and PSC tissue compared to normal tissue. In PBC and PSC samples, CAPN1 overall staining was generally greater in PBC samples compared with PSC (FIG. 4). In PSC, strong staining of the bile duct epithelium was observed. In subjects with intense inflammation, immunoreactivity was nearly ubiquitous.

### CAPN2

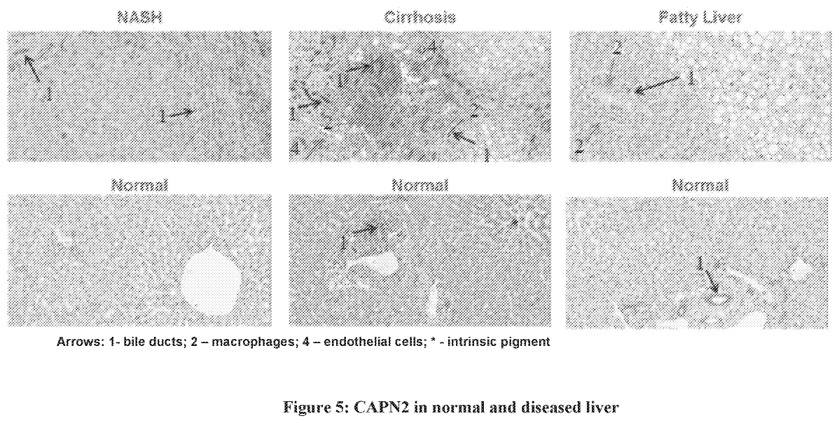

While CAPN2 reactivity occurred in bile duct epithelial cells regardless of disease status, CAPN2 immunoreactivity was increased in diseased tissue compared to normal tissue (FIG. 5). In diseased liver with inflammation, inflammatory cells (predominantly macrophages) and endothelial cells were variably CAPN2 positive. Hepatocytes adjacent to bands of fibrosis were CAPN2 positive with gradual loss toward the center of nodules or lobes.

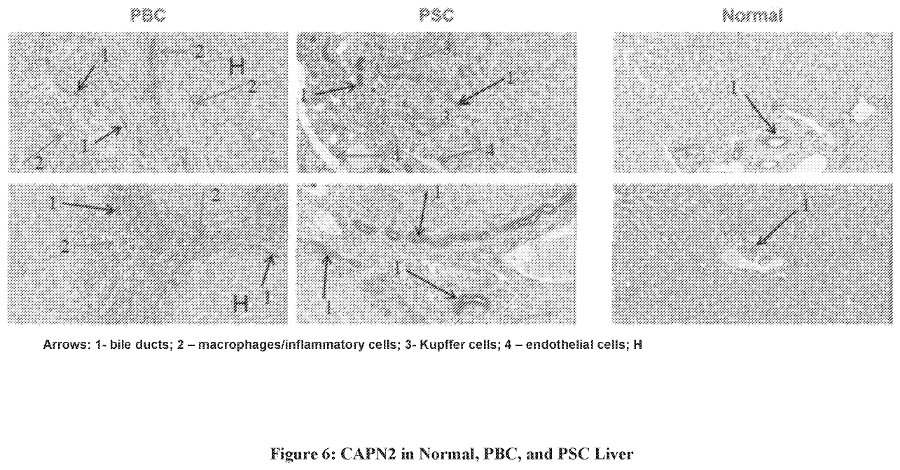

In PBC and PSC samples, CAPN2 reactivity was increased compared to normal tissue. CAPN2 positive bile duct epithelial varied in intensity without considerable differences between PBC and PSC (FIG. 6). In diseased liver with inflammation, inflammatory cells (predominantly macrophages) and endothelial cells were variably CAPN2 positive. Hepatocytes were CAPN2 positive in some subjects with wide variability. Subjects with the greatest inflammation tended to have the most intense stain.

### CAPN9

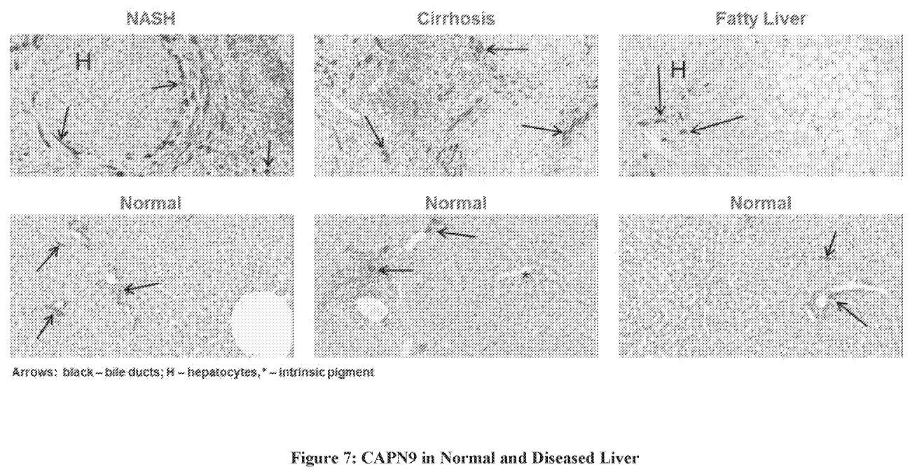

Strong CAPN9 reactivity occurred in bile duct epithelial cells regardless of disease status (FIG. 7). However, increased CAPN9 reactivity was observed in PBC and PSC tissue compared to normal tissue. Diseased liver typically included bile duct hyperplasia, which increased the overall CAPN9 positive cell distribution. Hepatocytes in diseased liver had mildly increased CAPN9 reactivity compared with healthy liver. In liver diagnosed as normal, some areas of fatty change (steatosis) had increased hepatocellular CAPN9 reactivity. Many sections had mild intrinsic hepatocellular pigment observed on isotype controls.

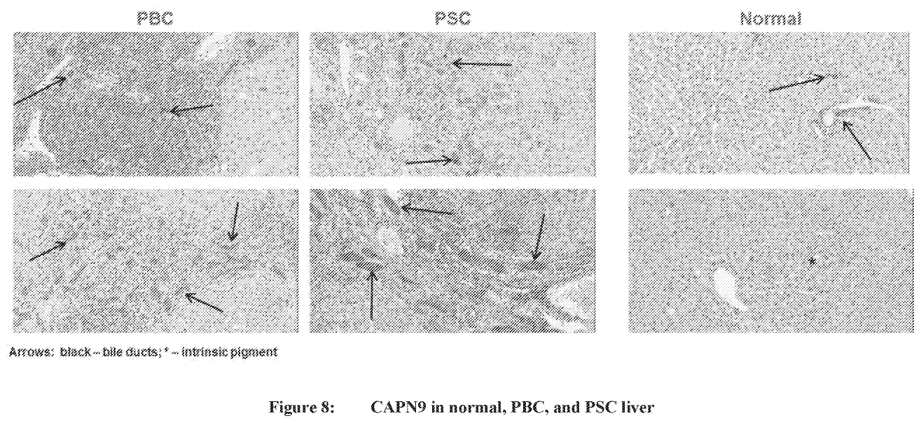

In PBC and PSC samples, CAPN9 reactivity was increased compared to normal tissue. Bile duct epithelial cells showed strong CAPN9 reactivity (FIG. 8). Little difference was noted between PBC and PSC. Diseased liver typically included bile duct hyperplasia, which increased the overall CAPN9 positive cell distribution. CAPN9 reactivity was strongest in degenerated hepatocytes.

### Example C

### Animal Models of NASH

A rat choline-deficient, amino acid-defined high fat diet (CDAHFD) model of non-alcoholic steatohepatitis (NASH) reproduces key features of the human disease. This model was used to examine the anti-fibrotic effects of Compound 405 (shown in Table 1a) in the CDAHFD rat model.

Male Wistar rats (Charles River Laboratories) were randomly assigned to receive the following treatments: Group 1 (n=8) served as a healthy control and were fed normal chow for 12 weeks; Group 2 (n=8) served as a disease control and was fed a CDAHFD and received once daily (QD) oral gavage treatment with vehicle Methylcellulose (MC) beginning at week 5 post CDAHFD, Group 3 (n=8) served as a disease control and was fed a CDAHFD and received twice daily (BID) oral gavage treatment with vehicle Methylcellulose (MC) beginning at week 5 post CDAHFD, Group 4 (n=8) was fed a CDAHFD and received QD oral gavage treatment with Compound 405 (200 mg/kg) beginning at week 5 post CDAHFD, Group 5 (n=8) was fed a CDAHFD and received BID oral gavage treatment with Compound 405 (100 mg/kg) beginning at week 5 post CDAHFD, Group 6 (n=8) was fed a CDAHFD and received QD oral gavage treatment with Compound 405 (60 mg/kg) beginning at week 5 post CDAHFD, Group 7 (n=8) was fed a CDAHFD and received BID oral gavage treatment with Compound 405 (30 mg/kg) beginning at week 5 post CDAHFD. At the end of the study (12 weeks), liver tissue and serum was collected for further analysis. A part of the liver was used for histological analysis and the spare liver from each individual animal was snap frozen on liquid nitrogen and stored at −80° C. Serum was collected to measure ALT,

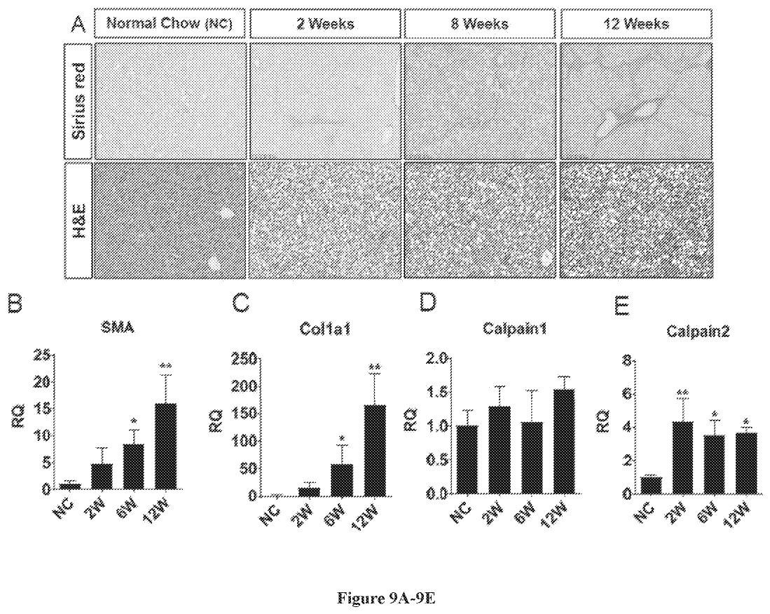

Rats fed CDAHFD developed liver fibrosis (F3) after 5 weeks of CDAHFD diet, which progressed to cirrhosis (F4) by 12 weeks (FIG. 9A). In this model as fibrosis progress, α-SMA and collagen1a1 expression increase gradually over time in rats fed CDAHFD (FIGS. 9B and 9C) Expression of Calpain 2 in liver increased in rats fed CDAHFD (NC 1.00±0.14, 2 weeks 4.34±1.38 **p<0.001, 6 weeks 3.50±0.90 *p<0.05 and 12 weeks, 3.67±0.32 *p<0.05, compared to NC) while the expression of Calpain 1 did not change over the course of this study (FIGS. 9D and 9E). Calpain 9 expression was not detectable by qPCR in rat liver.

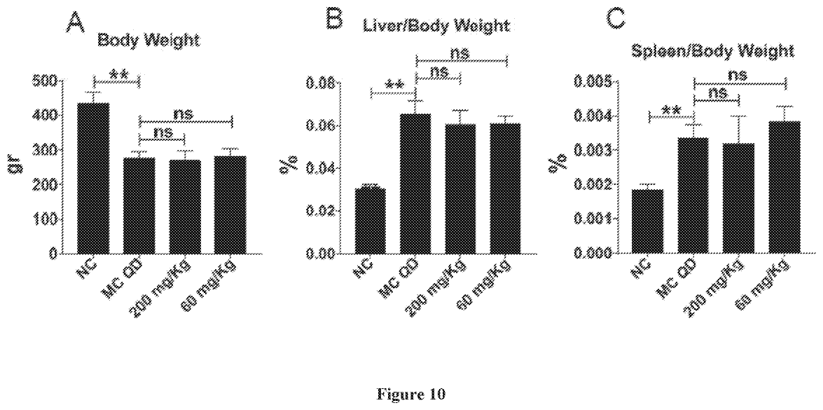

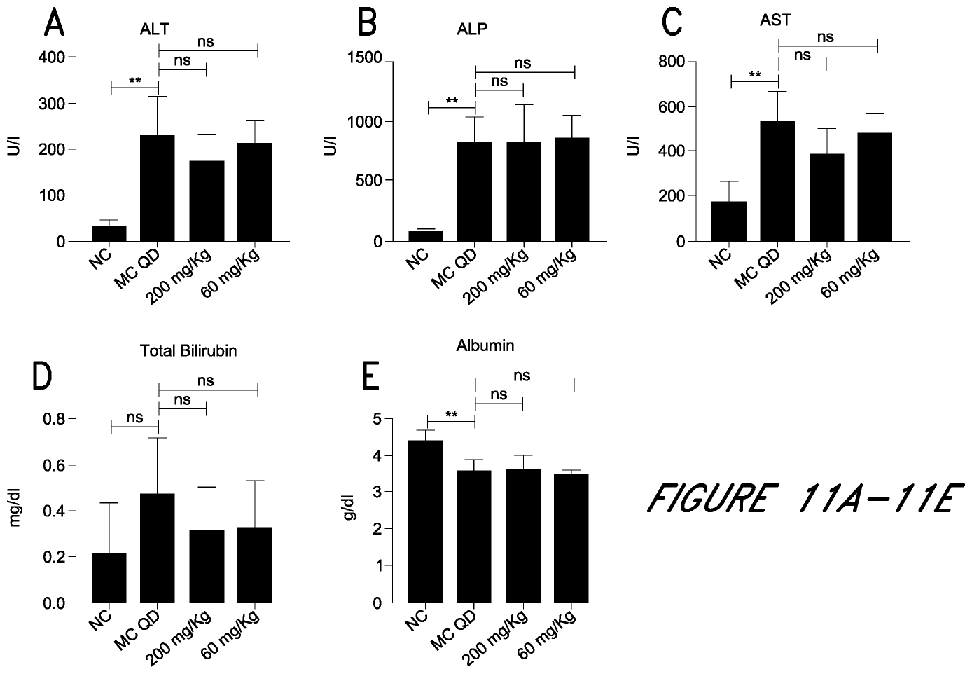

Body weight decreased in rats fed with CDAHFD compared to Normal chow fed rats. QD treatment with Compound 405 (200 mg/kg and 60 mg/kg) did not alter body weight in CDAHFD rats relative to vehicle (methyl cellulose) control rats. Liver weight and spleen weight (as a percent of total weight) were significantly higher in rats fed with CDAHFD compared to Normal chow (FIGS. 10A-10C). The level of blood transaminases (U/L) including ALT, AST, and ALP significantly increases in all the CDAHFD fed rats compared to normal chow, however, Serum analysis revealed that compared to the vehicle treated group (MC QD) Compound 405 did not alter the level of blood transaminases. Albumin level decrease in all CDAHFD rats compared to normal chow rats however, Compound 405 did not change the albumin levels in all the CDAHFD fed rats (FIGS. 11A-11E). Total bilirubin did not change significantly.

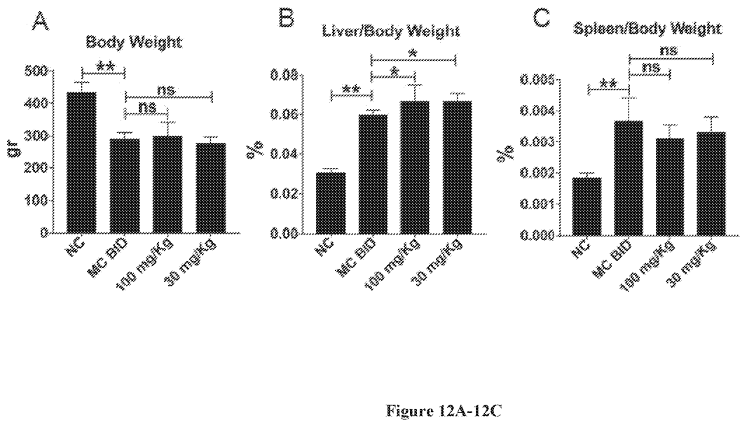

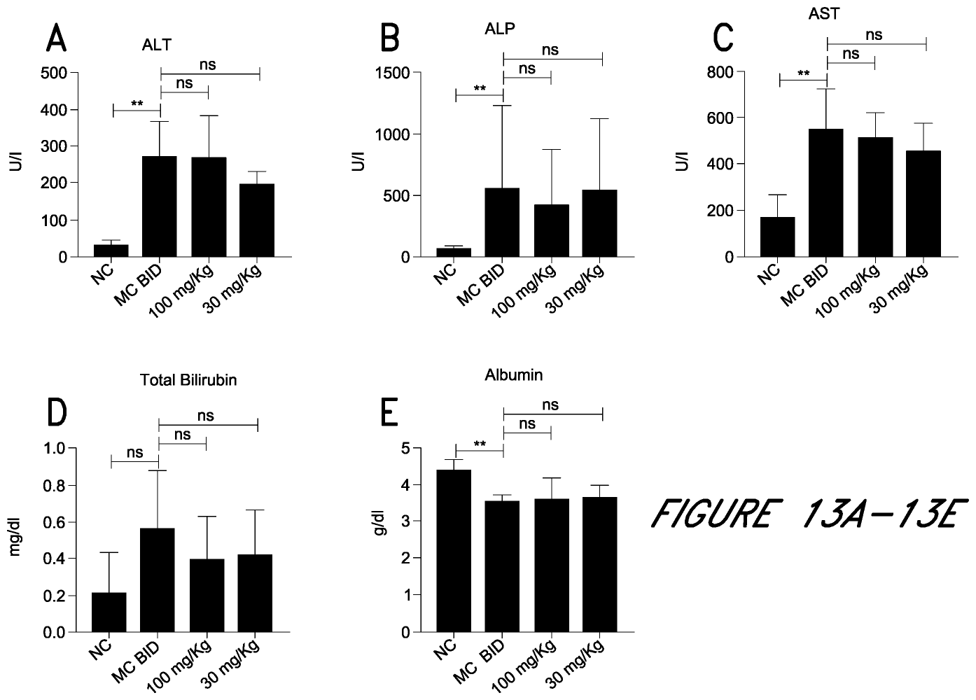

In the BID study, Compound 405 (100 mg/kg and 30 mg/kg) treatment did not affect body weight and spleen weight (as a percent of total weight) in CDAHFD rats. However, Liver Weight (as a percent of total weight) increased after treatment with Compound 405 (MC BID 0.059±0.002% vs. 100 mg/kg BID 0.066±0.008% and 30 mg/kg BID 0.066±0.003% *p<0.05) (FIGS. 12A-12C). The level of blood transaminases (U/L) including alanine transaminase (ALT), aspartate transaminase (AST), and ALP (alkaline phosphatase) increases in CDAHFD fed rats compared to normal chow (NC). But compared to the vehicle treated group (MC BID), Compound 405 did not change the level of blood transaminases (U/L) including ALT, AST, and ALP. Despite the decrease in albumin level in CDAHFD rats in comparison to normal chow (NC), Compound 405 did not alter albumin levels in CDAHFD fed rats (FIGS. 13A-13E). Total bilirubin did not change significantly.

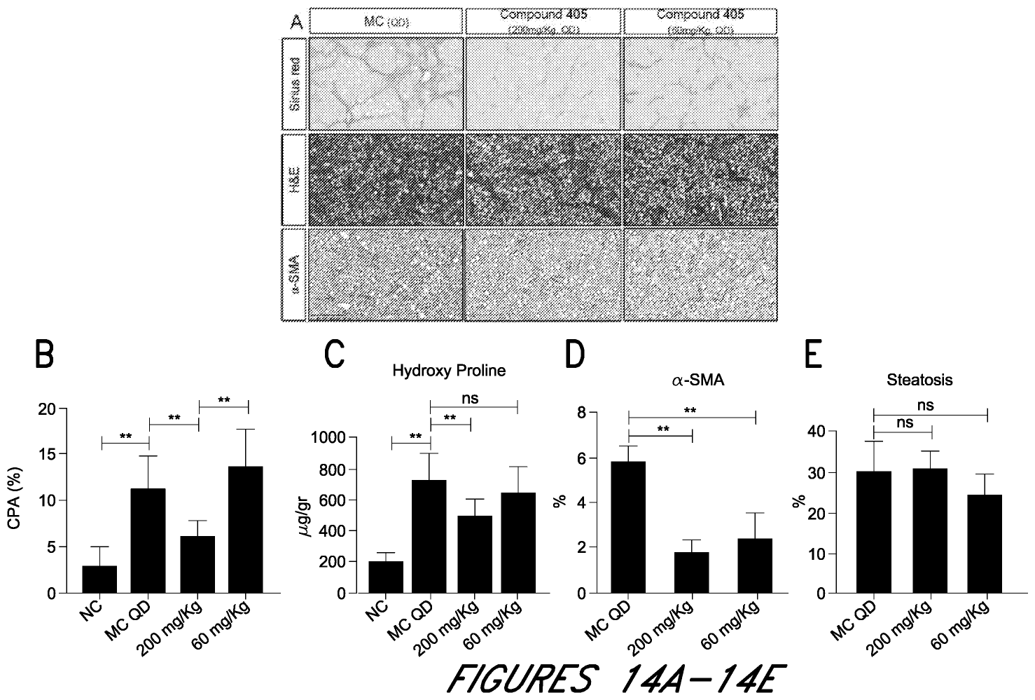

Representative histologic samples reveal bridging fibrosis in CDAHFD rats at 12 weeks (FIG. 14). Multiple methods of collagen quantitation were used to compare differences in liver fibrosis among treatment groups. By collagen proportional area (CPA) measurement, Compound 405 200 mg/kg QD significantly reduced collagen deposition as compared to MC QD in CDAHFD rats (11.28±3.50% vs. 6.08±1.69%, **p<0.05 (FIGS. 14A and 14B). Similar findings were also observed with hydroxyproline analysis. Compound 405 200 mg/kg QD treatment significantly reduced hydroxyproline (MC QD 731.3±165.9 nmol/L vs. 200 mg/kg QD 495.1±113.9 nmol/L *p<0.05) (FIG. 14C). Compound 405 60 mg/kg QD did not significantly decrease fibrosis as measured by CPA and hydroxyproline. However, SMA (Acta2) protein expression significantly decreased after treatment with both Compound 405 200 and 60 mg/kg QD compared with MC treated CDAHFD rats (MC QD 5.82±0.67 vs. 200 mg/kg 1.7±0.58% **p<0.01 and 60 mg/kg 2.40±1.16% **p<0.01) (FIGS. 14A and 14D). Histological analysis of steatosis showed that Compound 405 treatment QD did not change the liver fat content in CDAHFD rats (FIGS. 14A and 14E).

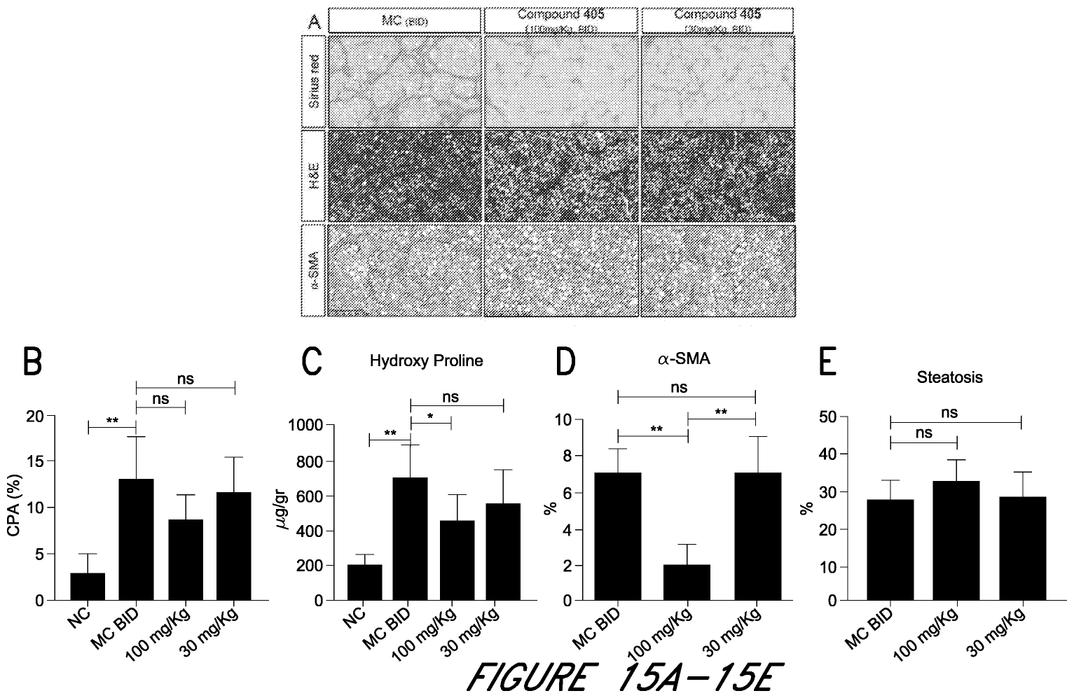

In the BID study, the same trend was present as Compound 405 100 mg/kg BID reduced collagen deposition in CDAHFD fed rats and significantly decreased hydroxyproline (MC BID 706.9±173.5 nmol/L vs. 100 mg/kg BID 458.2±146 nmol/L *p<0.05) (FIG. 15C). In addition, Compound 405 100 mg/kg BID significantly decreased SMA (Acta2) protein expression in livers in CDAHFD rats (MC BID 7.08±1.36% vs. 100 mg/kg BID 2.01±1.14%, **p<0.01) (FIGS. 15A and 15D). Histological analysis of steatosis showed that BID treatment of Compound 405 did not change the liver fat content in CDAHFD rats (FIGS. 15A and 15E).

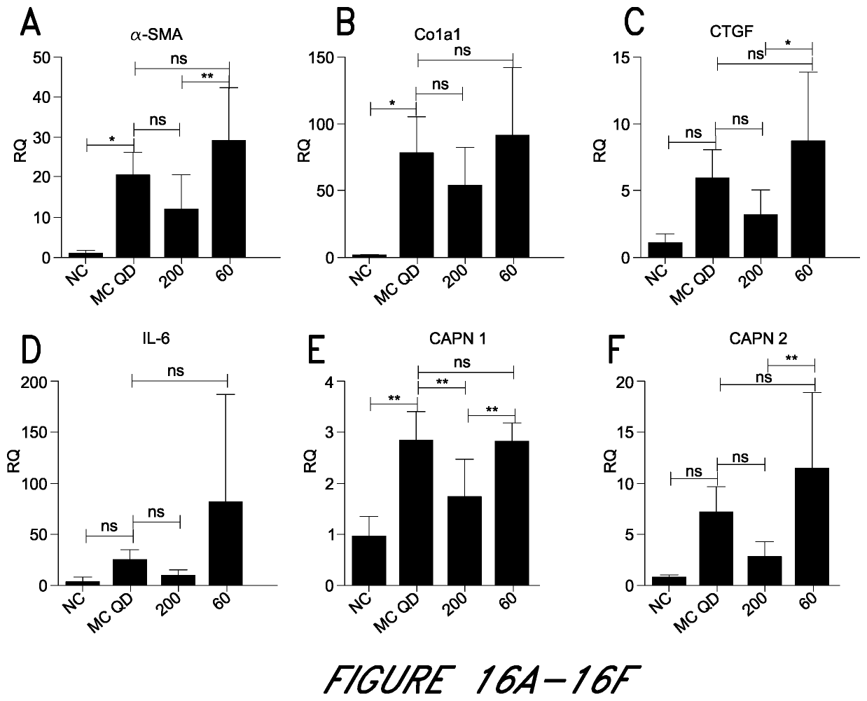

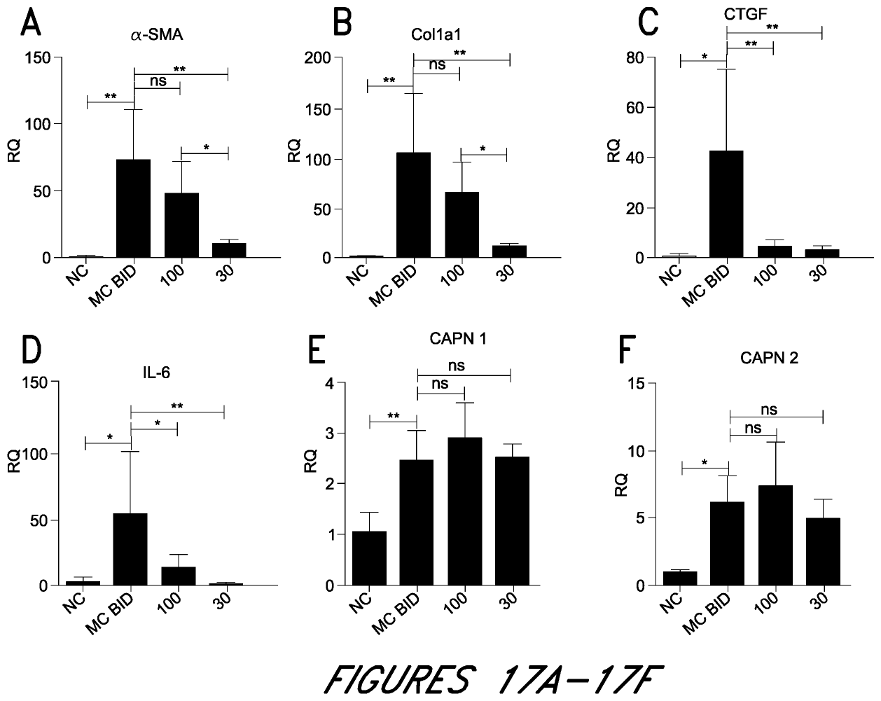

mRNA expression analysis showed that Compound 405 200 mg/kg or 60 mg/kg QD did not significantly decrease pro-fibrotic or inflammatory gene expression. (FIG. 16A-16F). By comparison, Compound 405 30 mg/kg BID decreased expression of SMA (MC BID 73.65±37.51 vs. 30 mg/kg 11.46±2.02 **p<0.01), and both Compound 405 100 and 30 mg/kg BID decreased expression of Colla1 (MC BID 106.6±59.97 vs. 100 mg/kg 35.76±33.84 **p<0.01 and 30 mg/kg 13.37±2.53 **p<0.01), CTGF (MC BID 42.89±32.82 vs. 100 mg/kg 3.84±2.24 *p<0.05 and 30 mg/kg 3.55±1.35 **p<0.01) and IL-6 (MC BID 54.89±47.23 vs. 100 mg/kg 15.81±9.01 **p<0.01 and 30 mg/kg 2.80±1.35 **p<0.01) (FIGS. 17A-17D). BID treatment of Compound 405 did not change the expression of Calpain1 and Calpain 2 (FIGS. 17E and 17F).

### Example D

### Animal Models of PSC

A model that is believed to mimic aspects of PSC is the mdr2−/− mouse model, as the model presents distinctive biliary fibrosis. The cells that are upregulating CAPN 1,2 and 9 in PSC, the bile duct epithelial cells, appear to be responsible for the development of fibrosis in this model.

Treatment compounds are tested in the Mdr2−/− mice on the fibrosis-susceptible BALB/c background, which spontaneously develop accelerated biliary fibrosis and early-onset portal hypertension (Ikenaga, N. et al (2015) A new Mdr2−/− mouse model of sclerosing cholangitis with rapid fibrosis progression, early-onset portal hypertension and liver cancer. Am J. Pathology 185 (2), 325-34). These mice develop fibrotic lesions and ductular reaction starting at 4 weeks of age, continuing to increase collagen deposition and early signs of cirrhosis at 12 weeks.

Mdr2−/− mice are dosed at 6 weeks of age for 6 weeks and the development of fibrosis is evaluated at week 12. Assessment at the end of the study includes hydroxyproline as a measure of overall fibrosis, histological analysis and gene expression of pro-fibrotic genes, including TGFß, procollagens, α-smooth muscle actin.

What is claimed is:

1. A method of treating a disease or disorder selected from the group consisting of primary sclerosing cholangitis, primary biliary cholangitis, non-alcoholic fatty liver disease, non-alcoholic steatohepatitis, and liver cirrhosis; the method comprising administering one or more calpain inhibitors to a subject in need thereof, wherein the calpain inhibitor is a compound of Formula I: 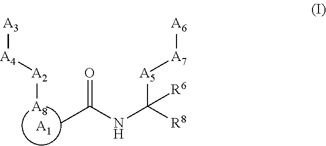
or a pharmaceutically acceptable salt thereof, wherein:
A₁ is selected from the group consisting of optionally substituted 5-10 membered heterocyclyl provided the 5-10 membered heterocyclyl is not substituted with oxo, optionally substituted 5-, 8-, or 9-membered heteroaryl, and optionally substituted C₃₋₁₀ carbocyclyl;
A₂ is selected from the group consisting of optionally substituted 3-10 membered heterocyclyl, optionally substituted C₆₋₁₀ aryl, optionally substituted 5-10 membered heteroaryl, and optionally substituted C₃₋₁₀ carbocyclyl, —CR₂—, —S—, —S(═O)—, —SO₂—, —O—, —C(═S)—, —C(═O)—, —NR—, —CH═CH—, —C≡C—, —OC(O)NH—, —NHC(O)NH—, —NHC(O)O—, —NHC(O)—, —NHC(S)NH—, —NHC(S)O—, —NHC(S)—, and single bond;
A₄ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₄ alkyl, —(CR₂)ₙ—S—(CR₂)ₙ—, —(CR₂)ₙ—S(═O)—(CR₂)ₙ—, —(CR₂)ₙ—SO₂—(CR₂)ₙ—, —(CR₂)ₙ—O—(CR₂)ₙ—, —(CR₂)ₙ—C(═S)—(CR₂)ₙ—, —(CR₂)ₙ—C(═O)—(CR₂)ₙ—, —(CR₂)ₙ—NR—(CR₂)ₙ—, —(CR₂)ₙ—CH═CH—(CR₂)ₙ—, —(CR₂)ₙ—OC(O)NH—(CR₂)ₙ—, —(CR₂)ₙ—NHC(O)NH—(CR₂)ₙ—, —(CR₂)ₙ—NHC(O)O—(CR₂)ₙ—, —(CR₂)ₙ—NHC(O)—(CR₂)ₙ—, —(CR₂)ₙ—NHC(S)NH—(CR₂)ₙ—, —(CR₂)ₙ—NHC(S)O—(CR₂)ₙ—, —(CR₂)ₙ—NHC(S)—(CR₂)ₙ—, and single bond;
when A₂ and A₄ are single bond, A₃ is directly attached to A₈;
A₃ is selected from the group consisting of optionally substituted C₆₋₁₀ aryl, optionally substituted 5-10 membered heteroaryl, optionally substituted 3-10 membered heterocyclyl, and optionally substituted C₃₋₁₀ carbocyclyl, or if A₂ is selected from optionally substituted 3-10 membered heterocyclyl, optionally substituted C₆₋₁₀ aryl, optionally substituted 5-10 membered heteroaryl, and optionally substituted C₃₋₁₀ carbocyclyl, then A₃ is selected from the group consisting of hydrogen, optionally substituted C₆₋₁₀ aryl, optionally substituted 5-10 membered heteroaryl, optionally substituted 3-10 membered heterocyclyl, optionally substituted C₃₋₁₀ carbocyclyl, —C≡CH, and optionally substituted 2- to 5-membered polyethylene glycol;
A₅ is selected from the group consisting of optionally substituted 3-10 membered heterocyclyl, optionally substituted C₆₋₁₀ aryl, optionally substituted 5-10 membered heteroaryl, optionally substituted C₃₋₁₀ carbocyclyl, optionally substituted C₁₋₈ alkyl, —S—, —S(═O)—, —SO₂—, —O—, —C(═S)—, —C(═O)—, —NR—, —CH═CH—, —OC(O)NH—, —NHC(O)NH—, —NHC(O)O—, —NHC(O)—, —NHC(S)NH—, —NHC(S)O—, —NHC(S)—, and single bond;
A₆ is selected from the group consisting of optionally substituted C₆₋₁₀ aryl, optionally substituted 5-10 membered heteroaryl, optionally substituted 3-10 membered heterocyclyl, and optionally substituted C₃₋₁₀ carbocyclyl, optionally substituted C₁₋₈ alkyl, optionally substituted C₂₋₈ alkenyl, optionally substituted —O—C₁₋₆ alkyl, optionally substituted —O C₂₋₆ alkenyl, —OSO₂CF₃, and any natural or non-natural amino acid side chain;
A₇ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₈ alkyl, —S—, S(═O)—, —SO₂—, —O—, —C(═S)—, —C(═O)—, —NR—, —CH═CH—, —OC(O)NH—, —NHC(O)NH—, —NHC(O)O—, —NHC(O)—, —NHC(S)NH—, —NHC(S)O—, —NHC(S)—, and single bond;
when A₅ and A₇ are single bond, A₆ is directly attached to the carbon to which R⁸ is attached;
A₈ is a ring member of A₁ and selected from the group consisting of C, CH, and N;
R⁸ is selected from the group consisting of —COR¹, —CN, —CH═CHSO₂R, and —CH₂NO₂;
R¹ is selected from the group consisting of H, —OH, C₁₋₄ haloalkyl, —COOH, —CH₂NO₂, —C(═O)NOR, —NH₂, —CONR²R³, —CH(CH₃)═CH₂, —CH(CF₃)NR²R³, —C(F)═CHCH₂CH₃, 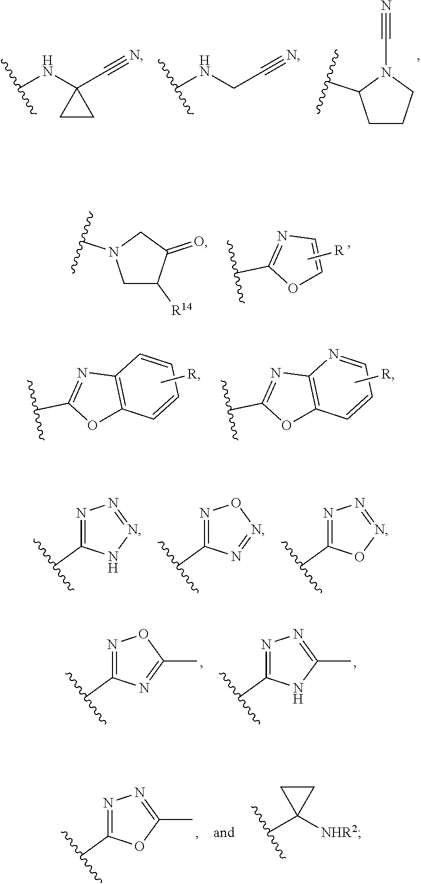
R¹⁴ is halo;
each R, R², and R³ are independently selected from —H, optionally substituted
C₁₋₄ alkyl, optionally substituted C₁₋₈ alkoxyalkyl, optionally substituted 2- to 5-membered polyethylene glycol, optionally substituted C₃₋₇ carbocyclyl, optionally substituted 5-10 membered heterocyclyl, optionally substituted C₆₋₁₀ aryl, and optionally substituted 5-10 membered heteroaryl; and
R⁶ is independently selected from —H and optionally substituted C₁₋₄ alkyl.

2. The method of claim 1, wherein A₁ is optionally substituted 5-10 membered heterocyclyl provided the 5-10 membered heterocyclyl is not substituted with oxo.

3. The method of claim 1 or claim 2, wherein A₁ is optionally substituted furyl, optionally substituted thienyl, optionally substituted phthalazinyl, optionally substituted pyrrolyl, optionally substituted oxazolyl, optionally substituted thiazolyl, optionally substituted imidazolyl, optionally substituted pyrazolyl, optionally substituted isoxazolyl, optionally substituted isothiazolyl, optionally substituted triazolyl, optionally substituted thiadiazolyl, optionally substituted pyridinyl, optionally substituted pyridazinyl, optionally substituted pyrimidinyl, optionally substituted pyrazinyl, optionally substituted triazinyl, optionally substituted quinolinyl, optionally substituted isoquinlinyl, optionally substituted benzimidazolyl, optionally substituted benzoxazolyl, optionally substituted benzothiazolyl, optionally substituted indolyl, optionally substituted isoindolyl, or optionally substituted benzothienyl.

4. The method of any one of claims 1 to 3, wherein A₁ is optionally substituted oxazolyl, optionally substituted thiazolyl, optionally substituted imidazolyl, optionally substituted pyrazolyl, or optionally substituted isoxazolyl.

5. The method of claim 1, wherein A₁ is an optionally substituted 5-, 8-, or 9-membered heteroaryl.

6. The method of any one of claims 1 to 5, wherein A₂ is a single bond.

7. The method of any one of claims 1 to 5, wherein A₄ is a single bond.

8. The method of any one of claims 1 to 7, wherein A₃ is selected from t optionally substituted C₆₋₁₀ aryl, optionally substituted 5-10 membered heteroaryl, optionally substituted 3-10 membered heterocyclyl, and optionally substituted C₃₋₁₀ carbocyclyl.

9. The method of claim 8, wherein A₃ is optionally substituted C₆₋₁₀ aryl.

10. The method of claim 9, wherein A₃ is optionally substituted phenyl.

11. The method of claim 8, wherein A₃ is optionally substituted 5-10 membered heteroaryl.

12. The method of any one of claims 1 to 11, wherein A₅ is optionally substituted C₁₋₈ alkyl.

13. The method of any one of claims 1 to 12, wherein A₇ is a single bond.

14. The method of any one of claims 1 to 13, wherein A₆ is selected from the group consisting of optionally substituted C₆₋₁₀ aryl, optionally substituted 5-10 membered heteroaryl, optionally substituted 3-10 membered heterocyclyl, and optionally substituted C₃₋₁₀ carbocyclyl.

15. The method of claim 14, wherein A₆ is optionally substituted C₆₋₁₀ aryl.

16. The method of claim 15, wherein A₆ is optionally substituted phenyl.

17. The method of claim 14, wherein A₆ is optionally substituted 5-10 membered heteroaryl.

18. The method of any one of claims 1 to 17, wherein R⁸ is —COR¹ and R¹ is selected from the group consisting of —COOH, —C(═O)NOR, or —CONR²R³.

19. The method of claim 18, wherein each R, R², and R³ are independently selected from —H and optionally substituted C₁₋₄ alkyl.

20. The method of any one of claims 1 to 19, wherein R⁶ is independently selected from —H and optionally substituted C₁₋₄ alkyl.

21. A method of treating a disease or disorder selected from the group consisting of primary sclerosing cholangitis, primary biliary cholangitis, non-alcoholic fatty liver disease, non-alcoholic steatohepatitis, and liver cirrhosis; the method comprising administering one or more calpain inhibitors to a subject in need thereof, wherein the calpain inhibitor is selected from the group consisting of: 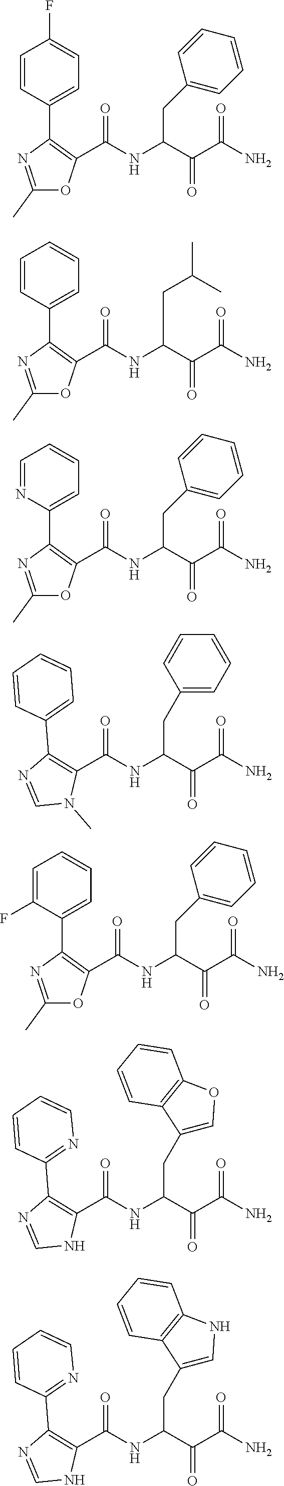 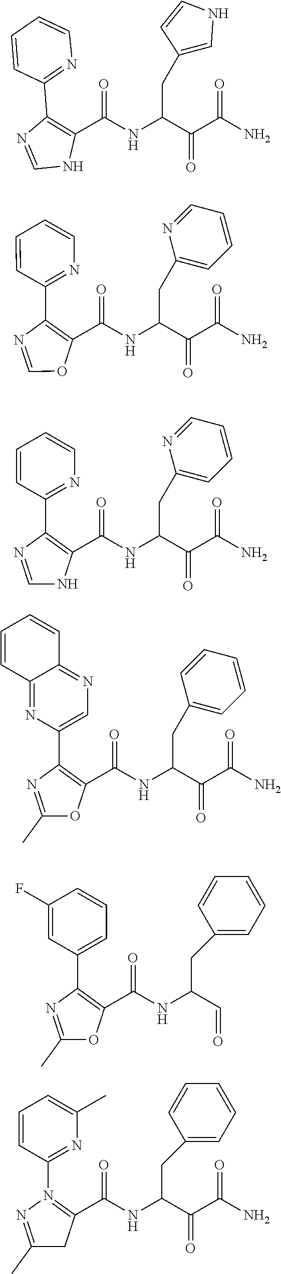 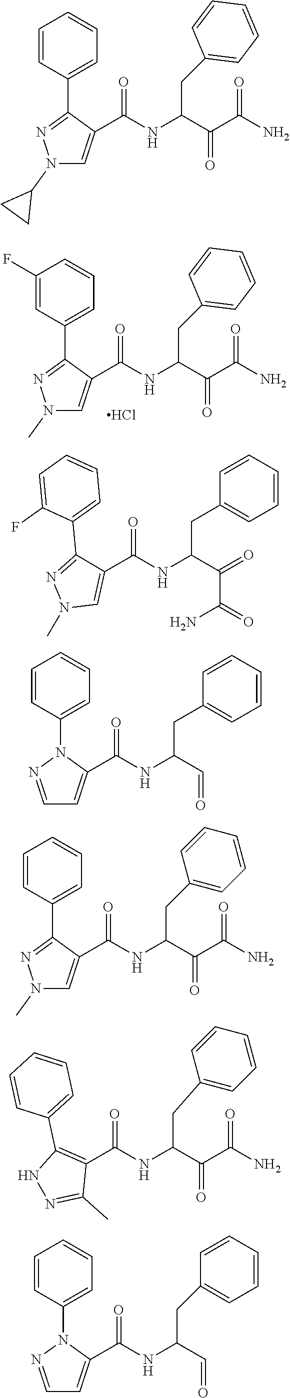 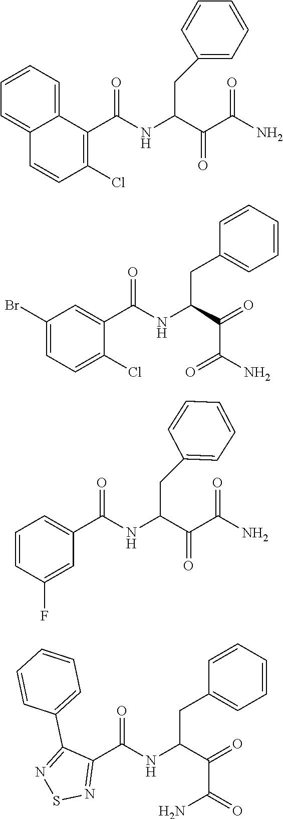
or pharmaceutically acceptable salts thereof.

22. The method of any one of claims 1 to 21, wherein the disease or disorder is non-alcoholic steatohepatitis.

23. The method of any one of claims 1 to 21, wherein the liver cirrhosis is caused by one or more of the conditions selected from the group consisting of alcoholic liver disease, alpha-1 antitrypsin deficiency, autoimmune hepatitis, celiac disease, chronic viral hepatitis, hemochromatosis, idiopathic portal fibrosis, and Wilson disease.

24. The method of any one of claims 1 to 23, wherein the calpain inhibitor is administered in combination with one or more additional agents selected from the group consisting of a VAP-1 inhibitor, an ASBT Inhibitor, a dual CCR2/5 antagonist, an anti-cholestatic bile acid, a FXR agonist, a FGFR1c/4 agonist, mesenchymal stem cell (MSC) cell therapy, a CCL24 Inhibitor, and a CCL11 inhibitor; and wherein the disease or disorder is primary sclerosing cholangitis.

25. The method of any of claims 1 to 23, wherein the calpain inhibitor is administered in combination with one or more additional agents selected from the group consisting of obeticholic acid, elafibranor, cenicriviroc, selonsertib, a niacin receptor agonist, a SGLT2 inhibitor, a VAP-1 inhibitor, a FGF21 mimetic, a adenosine A₃ receptor agonist, a mTOT modulator, a FXR agonist, a galectin-3 inhibitor, an ABCA1 activator, a SCD1 inhibitor, an ACC inhibitor, a Type I NK T-cell inhibitor, a pan-PPAR agonist, a DGAT2 inhibitor, a PPARalpha agonist, a thyroid hormone R-b agonist, a 5-LO/LT inhibitor, a mineralocorticoid receptor antagonist, a FGF19 mimic, a caspase inhibitor, a GLP-1R agonist, a SIRT1/AMP agonist, an ACC inhibitor, a ketohexokinase inhibitor, a GLP-1R agonist, an ASBT inhibitor, a DGAT2/CYP2E1 inhibitor, a TLR4 antagonist, a thyroid hormone R-b agonist, a IFN-gamma receptor antagonism, a CB1 antagonist, a FGF21 ligand, a P2Y13 receptor agonist, a CCL24 inhibitor, a MCH receptor-1 antagonist, aPPARalpha, delta agonist, a DPP-4 inhibitor, aLXR antagonist, a GLP1R agonist, an eotaxin-1 inhibitor, a beta-klotho/FGFR1c agonist, a LOXL2 Inhibitor, an AMPK activator, a miR-103/107 inhibitor, an inflammasome inhibitor, a CD3 antagonist, and a cathepsin B inhibitor; and wherein the disease or disorder is non-alcoholic steatohepatitis (NASH).
